(19) United States
(12) Patent Application Publication
(10) Pub. No.: US 2024/0177797 A1
(43) Pub. Date: May 30, 2024

(54) COMPOSITIONS AND METHODS FOR EXPRESSING GENES OF INTEREST IN HOST CELLS

(71) Applicant: Nobell Foods, Inc., South San Francisco, CA (US)

(72) Inventors: Viviane LANQUAR, San Carlos, CA (US); Leyla HATHWAIK, South San Francisco, CA (US); Yaxin WANG, South San Francisco, CA (US)

(21) Appl. No.: 18/519,995

(22) Filed: Nov. 27, 2023

Related U.S. Application Data
(63) Continuation of application No. PCT/US2022/031424, filed on May 27, 2022.
(60) Provisional application No. 63/194,424, filed on May 28, 2021.

(51) Int. Cl. G16B 15/10

(52) U.S. Cl. CPC G16B 15/10

(57) ABSTRACT

Provided herein are compositions and methods for stabilizing RNA, increasing protein expression, and combinations thereof. Also provided are compositions and methods for utilizing stabilized RNA or increased protein levels to generate chordate proteins in a host cell, such as a plant cell.

### CROSS REFERENCE TO RELATED APPLICATION

This application is a continuation of International Application No. PCT/US2022/031424 filed May 27, 2022, which claims priority to U.S. Provisional Patent Application No. 63/194,424 filed May 28, 2021, the contents of each of which are incorporated herein by reference in their entireties.

### REFERENCE TO AN ELECTRONIC SEQUENCE LISTING

The contents of the electronic sequence listing (ALRO_009_01US_SeqList_ST26.xml; Size: 1,019,910 bytes; and Date of Creation: Nov. 7, 2023) are herein incorporated by reference in its entirety.

### TECHNICAL FIELD

The present disclosure is related to compositions and methods for expressing exogenous proteins in host cells.

### BACKGROUND

The global population is estimated to reach 9.1-9.7 billion by 2050, with an approximately 1.8-fold increase in income per capita. Food consumption is predicted to become increasingly based on animal derived products—mainly due to increased wealth in developing countries. Thus, population growth and increased demand for high-protein diets will require dramatic changes in the food industry.

Proteins used as ingredients in the food industry have traditionally been based on isolates from natural sources, such bovine milk. However, it is rapidly becoming unsustainable to produce animal-derived food products, given their environmental impact and limited resources for production thereof. Recombinant protein production could provide an alternative source of protein for use in food applications. However, in order to make the use of recombinant proteins in food economically feasible, high levels of protein expression must be achieved in one or more host cells. Achieving such high expression levels poses numerous technical challenges.

Ovalbumin (OVAL) and β-Lactoglobulin (LG) are two proteins that play an important role in the food industry. OVAL is the main protein found in egg white that comprises 54% of the total protein. It is a globular monomeric protein that comprises a single polypeptide chain of 385 amino acids with a 42.7 kDa molecular weight which exist as a tetramer. OVAL is widely used in the food industry for its ability to foam and to form gels. On the other hand, LG is the major whey protein of milk in most mammals, making up approximately 52% of the whey protein. It is an 18.2 kDa highly structured globular protein that can exist as monomers, dimers and multimers depending on the pH of the medium. LG is a valuable ingredient in formulating food products because it can improve properties such as emulsification, gelling, biding and can provide increased nutritional value for health and wellbeing.

There is an urgent need to develop compositions and methods that allow for production of recombinant proteins at high levels in one or more host cells, so that the proteins may be used as ingredients in food compositions. Specifically, there is a need in the art to develop compositions and methods that increase expression of OVAL and LG in various transgenic organisms, cells, and the like.

### BRIEF SUMMARY

Provided is a method for selecting a nucleic acid sequence, said method comprising the steps of a) providing data on a plurality of nucleic acid sequences; b) predicting secondary structure of the plurality of nucleic acid sequences, with a plurality of RNA folding models, such that each nucleic acid sequence in the plurality of nucleic acid sequences is associated with at least two predicted secondary structures; c) determining a structural similarity score for the at least two predicted secondary structures associated with each nucleic acid sequence; and d) selecting a nucleic acid sequence with a higher structural similarity score than at least one other nucleic acid sequence in the plurality of nucleic acid sequences; wherein the selected nucleic acid sequence is predicted to accumulate at higher levels when expressed in a host cell.

Provided is a method for selecting a nucleic acid sequence, said method comprising the steps of: a) providing data on a plurality of nucleic acid sequences, each nucleic acid sequence in the plurality of nucleic acid sequences being associated with at least two predicted secondary structures from different RNA folding models; b) determining a structural similarity score for the at least two predicted secondary structures associated with each nucleic acid sequence; c) selecting a nucleic acid sequence with a higher structural similarity score than at least one other nucleic acid sequence in the plurality of nucleic acid sequences; wherein the selected nucleic acid sequence is predicted to accumulate at higher levels when expressed in a host cell. In some embodiments, at least one of the RNA folding models employs machine learning. In some embodiments, the plurality of nucleic acid sequences encode the same amino acid sequence. In some embodiments, the plurality of nucleic acid sequences encode amino acids sharing at least 90%, 91%, 92%, 93%, 94%, 95%, 96%, 97%, 98%, or 99% sequence identity. In some embodiments, the method comprises manufacturing the selected nucleic acid sequence into a nucleic acid. In some embodiments, the method comprises expressing the selected nucleic acid sequence in a host cell. In some embodiments, the method comprises expressing the manufactured nucleic acid in a host cell. In some embodiments, the nucleic acid sequence encodes for a messenger RNA. In some embodiments, the RNA folding models comprise a model selected from the group consisting of Cocke-Younger Kasami model, inside and outside models, loop-based energy model, minimum free energy, suboptimal folding, centroid, and any combination thereof. In some embodiments, the at least two predicted secondary structures are a minimum free energy structure and a centroid structure. In some embodiments, the structural similarity score is determined via tool selected from the group consisting of Consan, Dynalign, PMcomp, Stemloc, Foldalign, locARNA, SPARSE, MARNA, FoldAlignM, Murlet, CARNA, RAF, RNAforester, RNAdistance, RNAStrAt, RNApdist, and any combination thereof. In some embodiments, the structure similarity score is based on visual inspection of the predicted secondary structures. In some embodiments, the structure similarity score is a ranking of the plurality of nucleic acid sequences based on the relative similarity of each nucleic acid sequences' predicted secondary structures. In some embodiments, the similarity score is based on degree of curve overlap in a graph depicting number of base pairs at each position of the predicted secondary structures. In some embodiments, the similarity score is based on the degree of curve overlap of the predicted secondary structures plotted in a mountain plot. In some embodiments, the similarity score is based on the correlation of curves representing the predicted secondary structures in a graph depicting number of base pairs at each position. In some embodiments, the degree of curve overlap is calculated by methodology selected from the group consisting of least squares, curve length measure, and any combination thereof.

Provided is a method of manufacturing a nucleic acid, said method comprising: a) manufacturing a selected nucleic acid sequence to produce a nucleic acid, wherein the selection of the nucleic acid sequence was based on the selected nucleic acid sequence having a higher structural similarity score than at least one other nucleic acid sequence in a plurality of nucleic acid sequences; wherein the structural similarity score is based on the structural similarity between at least two predicted secondary structures for each nucleic acid sequence, the predicted secondary structures produced by different RNA folding models. In some embodiments, at least one of the RNA folding models employs machine learning. In some embodiments, the plurality of nucleic acid sequences encode the same amino acid sequence. In some embodiments, the plurality of nucleic acid sequences encode amino acids sharing at least 90%, 91%, 92%, 93%, 94%, 95%, 96%, 97%, 98%, or 99% sequence identity. In some embodiments, the method comprises expressing the manufactured nucleic acid in a host cell. In some embodiments, the manufactured nucleic acid expresses at a higher level than other nucleic acids containing other nucleic acid sequences from the plurality of nucleic acid sequences. In some embodiments, the RNA folding models comprise a model selected from the group consisting of Cocke-Younger Kasami model, inside and outside models, loop-based energy model, minimum free energy, suboptimal folding, centroid, and any combination thereof. In some embodiments, the at least two predicted secondary structures are a minimum free energy structure and a centroid structure. In some embodiments, the structural similarity score is determined via tool selected from the group consisting of Consan, Dynalign, PMcomp, Stemloc, Foldalign, locARNA, SPARSE, MARNA, FoldAlignM, Murlet, CARNA, RAF, RNAforester, RNAdistance, RNAStrAt, RNApdist, and any combination thereof. In some embodiments, the structure similarity score is based on visual inspection of the predicted secondary structures. In some embodiments, the structure similarity score is a ranking of the plurality of nucleic acid sequences based on the relative similarity of each nucleic acid sequences' predicted secondary structures. In some embodiments, the similarity score is based on degree of curve overlap in a graph depicting number of base pairs at each position of the predicted secondary structures. In some embodiments, the similarity score is based on the degree of curve overlap of the predicted secondary structures plotted in a mountain plot.

Provided is a nucleic acid comprising a nucleic acid sequence selected in a method of the disclosure.

Provided is a host cell comprising a nucleic acid comprising a sequence of Table 11, Table 12, or Table 15. In some embodiments, the nucleic acid comprises a sequence selected from the group consisting of: SEQ ID NO: 757, 760, 762, 763, 765, 772, 773, 778, and 780.

Provided is a host cell comprising a nucleic acid encoding any one of SEQ ID NO: 685, 687, and 695.

Provided herein is a host cell that comprises an exogenous RNA sequence that encodes a chordate protein, wherein the exogenous RNA sequence is stabilized as determined by increased expression of the chordate protein as compared to an otherwise comparable host cell lacking the exogenous RNA sequence that is stabilized, and wherein the chordate protein is expressed in the amount of at least 1% or higher per total protein weight of soluble protein extractable from the host cell.

In some embodiments, the chordate is a vertebrate. In some embodiments, the vertebrate is a mammal. In some embodiments, the mammal is a bovine. In some embodiments, the vertebrate is a bird. In some embodiments, the bird is a chicken.

In some embodiments, the chordate protein is an egg protein or a milk protein. In some embodiments, the chordate protein is a milk protein. In some embodiments, the milk protein is β-lactoglobulin. In some embodiments, the chordate protein is an egg protein. In some embodiments, the egg protein is ovalbumin. In some embodiments, the chordate protein is expressed in the amount of at least 2%, at least 3%, at least 4%, or at least 5% per total protein weight of soluble protein extractable from the host cell. In some embodiments, the chordate protein is expressed in the amount of about 1 to about 2%, about 2 to about 3%, or about 2 to about 5% per total protein weight of soluble protein extractable from the host cell.

In some embodiments, provided herein is a plant that comprises a host cell. In some embodiments, the plant is a soybean plant.

In some embodiments, provided is a DNA construct for expression of a transgene in a host cell, wherein the DNA construct comprises: (a) a codon-optimized transgene sequence that comprises at least 90% identity to a sequence selected from the group consisting of SEQ ID NO: 1, 3, 5, 7, 9-13, 83, 617-621, 683-690, and 693-700; and (b) an exogenous intron sequence, wherein the exogenous intron sequence comprises at least 90% identity to a sequence selected from the group consisting of: SEQ ID NO: 679-682. In some embodiments, the codon-optimized transgene sequence comprises a sequence selected from SEQ ID NO: 1, 3, 5, 7, 9-13, 83, 617-621, 683-690, and 693-700. In some embodiments, the exogenous intron sequence comprises a sequence selected from SEQ ID NO: 679-682.

In some embodiments, provided is a DNA construct for expression of a transgene in a host cell, wherein the DNA construct comprises: (a) a codon-optimized transgene sequence that comprises at least 90% identity to a sequence selected from the group consisting of SEQ ID NO: 1, 3, 5, 7, 9-13, 83, 617-621, 683-690, and 693-700; and (b) an exogenous intron sequence, wherein the exogenous intron sequence comprises at least 90% identity to a sequence selected from the group consisting of: SEQ ID NO: 679-682. In some embodiments, the codon-optimized transgene sequence comprises a sequence selected from SEQ ID NO: 1, 3, 5, 7, 9-13, 83, 617-621, 683-690, and 693-700. In some embodiments, the exogenous intron sequence comprises a sequence selected from SEQ ID NO: 679-682. In some embodiments, the DNA construct further comprises a signal peptide sequence. In some embodiments, the signal peptide sequence is selected from the group consisting of: SEQ ID NO: 616, 707-717. In some embodiments, the DNA construct further comprises a sequence encoding a KDEL sequence. In some embodiments, the DNA construct further comprises a sequence encoding at least one of a 5′ UTR and a 3′ UTR. In some embodiments, the DNA construct further comprises a sequence encoding a ubiquitin monomer. In some embodiments, the DNA construct further comprises an exogenous promoter sequence. In some embodiments, the exogenous promoter sequence is isolated or derived from a plant promoter sequence. In some embodiments, the exogenous promoter sequence is isolated or derived from a seed promoter sequence. In some embodiments, the DNA construct further comprises an exogenous terminator sequence.

Provided herein is also a composition that comprises a DNA construct.

Provided herein is also a method of transforming a host cell, the method comprising contacting a host cell with a composition provided herein, thereby transforming the host cell. In some embodiments, the host cell is a plant cell. In some embodiments, the method comprises bombardment or agrobacterium-mediated transformation. In some embodiments, the method further comprises cultivating the plant cell after the transforming.

Provided herein is an RNA generated from a DNA construct provided herein.

Provided herein is also a method of expressing ovalbumin or β-lactoglobulin in a plant, the method comprising: contacting at least a portion of a plant with a DNA construct of the disclosure, wherein the method is effective in increasing expression of the ovalbumin or β-lactoglobulin as compared to an otherwise comparable method lacking the contacting. In some embodiments, the method is effective in increasing expression of the ovalbumin or β-lactoglobulin by at least about 1-fold as compared to an otherwise comparable method lacking the contacting.

Provided herein is also a method of stably expressing a chordate protein in a plant cell, the method comprising: (a) contacting a plant cell with a DNA construct that comprises at least 90% identity to a sequence selected from the group consisting of SEQ ID NO: 752-766, thereby generating a transformed plant cell; and (b) cultivating a plant that comprises the transformed plant cell, thereby generating a transformed plant, wherein the chordate protein is expressed in the amount of 1% or higher per total protein weight of soluble protein extractable from the transformed plant cell. In some embodiments, the DNA construct comprises at least 95%, at least 97%, or at least 99% identity to a sequence selected from the group consisting of SEQ ID NO: 752-766.

Provided herein is also a method of stably expressing a chordate protein in a plant cell, the method comprising: (a) contacting a plant cell with a DNA construct that comprises at least 90% identity to a sequence selected from the group consisting of SEQ ID NO: 767-781, thereby generating a transformed plant cell; and (b) cultivating a plant that comprises the transformed plant cell, thereby generating a transformed plant, wherein the chordate protein is expressed in the amount of 1% or higher per total protein weight of soluble protein extractable from the transformed plant cell. In some embodiments, the DNA construct comprises at least 95%, at least 97%, or at least 99% identity to a sequence selected from the group consisting of SEQ ID NO: 767-781. In some embodiments, the chordate protein is expressed in the amount of at least 1%, at least 2%, at least 3%, at least 4%, or at least 5% per total protein weight of soluble protein extractable from the transformed plant cell. In some embodiments, the plant cell is from a soybean plant. In some embodiments, the contacting comprises bombardment or agrobacterium-mediated transformation. In some embodiments, a level of a transcript of a transgene encoded by the DNA construct is increased by at least 1-fold as compared to an otherwise comparable method lacking the contacting. In some embodiments, a level of the chordate protein encoded by the DNA construct is increased by at least 1-fold as measured by ELISA and as compared to an otherwise comparable method lacking the contacting. In some embodiments, the level is increased by at least 3-fold, at least 5-fold, at least 10-fold, at least 30-fold, or at least 50-fold. In some embodiments, the method further comprises isolating a seed from the transformed plant.

Provided herein is also a nutraceutical that comprises a chordate protein isolated from a transformed plant cell generated by a method of the disclosure.

### BRIEF DESCRIPTION OF THE DRAWINGS

The accompanying figures, which are incorporated herein and form a part of the specification, illustrate some, but not the only or exclusive, example embodiments and/or features. It is intended that the embodiments and figures disclosed herein are to be considered illustrative rather than limiting.

FIG. 1 is a schematic showing an exemplary strategy to compare RNA stabilization of constructs encoding ovalbumin (OVAL) and/or lactoglobulin (LG) and their respective protein data. Data was bundled using different categories in order to evaluate strategies and/or construct designs that can lead to RNA stability and higher protein accumulation.

FIG. 2A shows RNA expression of exemplary constructs encoding ovalbumin. FIG. 2B shows protein expression of exemplary constructs encoding ovalbumin as determined by ELISA. Protein expression is shown as a function of relative expression and percent total soluble protein (% TSP), respectively. Boxes indicate the designs that led to an increase in both RNA expression and protein accumulation. In between FIG. 2A and FIG. 2B, a breakdown of the designs based on “high level strategy” is shown. n=number of seeds analyzed. Note: Since the RNA level (cut off <0.1) was low for plants transformed with constructs AR07-22 and AR07-23 as well as AR15-18, -20, -23 and -38, the plants were discarded, and no protein data was collected for these constructs (n=0).

FIG. 3A shows gradient plots summarizing RNA expression of exemplary (3-Lactoglobulin designs. FIG. 3B shows gradient plots summarizing protein expression of exemplary β-Lactoglobulin designs. Protein expression is shown as a function of relative expression and % TSP, respectively. Boxes indicate the designs that led to an increase in both RNA expression and protein accumulation. In between FIG. 3A and FIG. 3B is a breakdown of the designs based on high level strategy. n=number of seeds analyzed. Cassette details: AR07-28 BnNap:sig11:OLG1:KDEL:nos, AR07-29 GmSeed2:sig2:OLG1:KDEL:nos, AR07-31 GmSeed12:coixss:OLG1:KDEL:nos, AR07-32 GmSeed12:sig12:OLG1:KDEL:nos, AR07-33 PvPhas:arcUTR:sig10:OLG1:KDEL:arcT, AR15-25 GmSeed2: sig2:OLG2:KDEL:EUT:Rb7T, AR15-26GmSeed2:sig2:OLG3:KDEL:EUT:Rb7T, AR15-27 GmSeed2:sig2:OLG4:KDEL:EUT:Rb7T, AR15-28 GmSeed2:sig2:OLG2:EUT:Rb7T, AR15-29 GmSeed2 (intron 1):sig2:OLG2:KDEL:EUT:Rb7T, AR15-30 GmSeed2:sig2:OLG2 (intron 1):KDEL:EUT:Rb7T, AR15-31 GmSeed2:sig2:OLG2 (intron 2):KDEL:EUT:Rb7T, AR15-36 GmSeed2:lgUTR:sig2:OLG2:KDEL:EUT:Rb7T, AR15-37 GmSeed2:glnB1UTR:sig2:OLG2: KDEL: EUT:Rb7T, AR15-39 GmSeed2:Ubimonomer:sig2:OLG2:KDEL:EUT:Rb7T. See also, Table 12.

FIG. 4 is a graphic of modified Gallus gallus ovalbumin gene that was used in various constructs described herein. Plant intron 1 or 2 was placed in the location of native intron 2-3.

FIG. 5 is a graphic of modified Bos taurus β-Lactoglobulin gene that was used in various constructs described herein. Plant intron 1 or 2 was placed in the location of native intron 1-2.

FIG. 6 is a graphic of the pAR15-00 cloning vector containing a selectable marker cassette conferring herbicide resistance. The pAR15-00 cloning vector is a modified binary pCAMBIA3300 vector containing the mutant acetolactate synthase gene (AtCsr1.2) of Arabidopsis thaliana driven by the StUbi3 promoter, which is followed by the StUbi3 terminator. A multiple cloning site (MCS) was included downstream of the selectable marker cassette. Within the MCS, a KpnI restriction enzyme site was available to insert the expression cassette into the pAR15-00 vector.

FIG. 7 is a graphic of the pAR07-00 cloning vector containing a selectable marker cassette conferring spectinomycin resistance in plants. The pAR07-00 cloning vector is a modified binary pCAMBIA3300 vector containing the Aminoglycoside-3″-adenyltransferase (aadA) gene fused to a petunia EPSPS chloroplast transit peptide (CTP), that confers resistance to spectinomycin driven by the 35S promoter, which is followed by the 35S terminator. A BamHI restriction enzyme site was available to insert the expression cassette into the vector between the antibiotic resistance gene and the mCherry marker gene.

FIG. 8—depicts the predicted minimum free energy secondary structures for several codon optimized β-Lactoglobulin-encoding nucleic acid sequences. Highest RNA expression among transformants also depicted. There was no obvious correlation between expression and any single secondary structure.

FIG. 9—depicts the predicted minimum free energy and centroid secondary structures for several codon optimized β-Lactoglobulin-encoding nucleic acid sequences. Highest RNA expression among transformants also depicted. It was observed that higher expressing sequences had similar predicted structures between different prediction algorithms.

FIG. 10A-FIG. 10F—depict the predicted minimum free energy (MFE) and centroid secondary structures for several codon optimized β-Lactoglobulin-encoding nucleic acid sequences. Mountain plot graphs for both the MFE and centroid structures are shown, together with curve length distance between the curves for each predicted secondary structure. Highest RNA expression among transformants based on empirical measurements, as well as overall RNA fold expression increase over lowest expressing sequence is shown.

FIG. 11A-FIG. 11F—depict the predicted minimum free energy (MFE) and centroid secondary structures for several codon optimized ovalbumin-encoding nucleic acid sequences. Mountain plot graphs for both the MFE and centroid structures are shown, together with curve length distance between the curves for each predicted secondary structure. Highest RNA expression among transformants based on empirical measurements, as well as overall RNA fold expression increase over lowest expressing sequence is shown.

FIG. 12A-FIG. 12F—depict the predicted minimum free energy (MFE) and centroid secondary structures for several codon optimized green fluorescent protein-encoding nucleic acid sequences. Mountain plot graphs for both the MFE and centroid structures are shown, together with curve length distance between the curves for each predicted secondary structure. Highest RNA expression among transformants based on empirical measurements, as well as overall RNA fold expression increase over lowest expressing sequence is shown.

FIG. 13A-B—depicts a X-Y scatter plot of curve length measure between predicted secondary structures for each nucleic acid sequence created from different RNA folding models, and highest RNA expression from constructs comprising each nucleic acid sequence. FIG. 13A depicts RNA expression in the X-Axis and curve length on the Y-Axis and includes a linear regression trendline for reference. FIG. 13 B depicts curve length measure in the X-Axis and RNA expression in the Y-Axis. A Logarithmic trendline is added for the correlation between the two variables.

FIG. 14—depicts a X-Y scatter plots of curve length measure between predicted secondary structures for each nucleic acid sequence created from different RNA folding models, and highest RNA expression from constructs comprising each nucleic acid sequence. Separate plots for β-Lactoglobulin, Ovalbumin, and Green Fluorescent Protein are provided, each with trend lines showing correlation.

### DETAILED DESCRIPTION

The following description includes information that may be useful in understanding the present disclosure. It is not an admission that any of the information provided herein is prior art or relevant to the presently claimed disclosures, or that any publication specifically or implicitly referenced is prior art.

Provided herein are compositions and methods for increasing expression levels in a host cell of one or more proteins encoded by transgenes by way of RNA stabilization. These compositions and methods may be used to express one or more proteins, such as ovalbumin (OVAL) and β-Lactoglobulin (LG), at high levels in a host cell. Also provided are various transgenic organisms, animals, crops, and cells that comprise stabilized RNA and/or enhanced levels of proteins encoded by the stabilized RNA. The compositions and methods may be used to generate transgenic cells, organisms, crops, animals, and the like, and to produce recombinant protein therein.

### Definitions

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one of skill in the art.

As used herein, the singular forms “a,” “an,” and “the: include plural referents unless the content clearly dictates otherwise.

The term “about” or “approximately” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. When used in conjunction with a range or series of values, the term “about” applies to the endpoints of the range or each of the values enumerated in the series, unless otherwise indicated. Similarly, the term “about” when preceding a series of numerical values or a range of values (e.g., “about 10, 20, 30” or “about 10-30”) refers, respectively to all values in the series, or the endpoints of the range.

As used herein, “mammalian milk” can refer to milk derived from any mammal, such as bovine, human, goat, sheep, camel, buffalo, water buffalo, dromedary, llama and any combination thereof. In some embodiments, a mammalian milk is a bovine milk.

As used herein, “rennet” refers to a set of enzymes typically produced in the stomachs of ruminant mammals. Chymosin, its key component, is a protease enzyme that cleaves x-casein (to produce para-κ-casein and a macropeptide). In addition to chymosin, rennet contains other enzymes, such as pepsin and lipase. Rennet is used to separate milk into solid curds (for cheesemaking) and liquid whey. Rennet or rennet substitutes are used in the production of many cheeses.

The term “plant” includes reference to whole plants, plant organs, plant tissues, and plant cells and progeny of same, but is not limited to angiosperms and gymnosperms such as Arabidopsis, potato, tomato, tobacco, alfalfa, lettuce, carrot, strawberry, sugar beet, cassava, sweet potato, soybean, lima bean, pea, chickpea, maize (corn), turf grass, wheat, rice, barley, sorghum, oat, oak, eucalyptus, walnut, palm and duckweed as well as fern and moss. Thus, a plant may be a monocot, a dicot, a vascular plant reproduced from spores such as fem or a nonvascular plant such as moss, liverwort, hornwort and algae. The word “plant,” as used herein, also encompasses plant cells, seeds, plant progeny, propagule whether generated sexually or asexually, and descendants of any of these, such as cuttings or seed. Plant cells include suspension cultures, callus, embryos, meristematic regions, callus tissue, leaves, roots, shoots, gametophytes, sporophytes, pollen, seeds and microspores. Plants may be at various stages of maturity and may be grown in liquid or solid culture, or in soil or suitable media in pots, greenhouses or fields. Expression of an introduced leader, trailer or gene sequences in plants may be transient or permanent.

The term “vascular plant” refers to a large group of plants that are defined as those land plants that have lignified tissues (the xylem) for conducting water and minerals throughout the plant and a specialized non-lignified tissue (the phloem) to conduct products of photosynthesis. Vascular plants include the clubmosses, horsetails, ferns, gymnosperms (including conifers) and angiosperms (flowering plants). Scientific names for the group include Tracheophyta and Tracheobionta. Vascular plants are distinguished by two primary characteristics. First, vascular plants have vascular tissues which distribute resources through the plant. This feature allows vascular plants to evolve to a larger size than non-vascular plants, which lack these specialized conducting tissues and are therefore restricted to relatively small sizes. Second, in vascular plants, the principal generation phase is the sporophyte, which is usually diploid with two sets of chromosomes per cell. Only the germ cells and gametophytes are haploid. By contrast, the principal generation phase in non-vascular plants is the gametophyte, which is haploid with one set of chromosomes per cell. In these plants, only the spore stalk and capsule are diploid.

The term “non-vascular plant” refers to a plant without a vascular system consisting of xylem and phloem. Many non-vascular plants have simpler tissues that are specialized for internal transport of water. For example, mosses and leafy liverworts have structures that look like leaves but are not true leaves because they are single sheets of cells with no stomata, no internal air spaces and have no xylem or phloem. Non-vascular plants include two distantly related groups. The first group are the bryophytes, which is further categorized as three separate land plant Divisions, namely Bryophyta (mosses), Marchantiophyta (liverworts), and Anthocerotophyta (hornworts). In all bryophytes, the primary plants are the haploid gametophytes, with the only diploid portion being the attached sporophyte, consisting of a stalk and sporangium. Because these plants lack lignified water-conducting tissues, they can't become as tall as most vascular plants. The second group is the algae, especially the green algae, which consists of several unrelated groups. Only those groups of algae included in the Viridiplantae are still considered relatives of land plants.

The term “plant part” refers to any part of a plant including but not limited to the embryo, shoot, root, stem, seed, stipule, leaf, petal, flower bud, flower, ovule, bract, trichome, branch, petiole, internode, bark, pubescence, tiller, rhizome, frond, blade, ovule, pollen, stamen, and the like. The two main parts of plants grown in some sort of media, such as soil or vermiculite, are often referred to as the “above-ground” part, also often referred to as the “shoots”, and the “below-ground” part, also often referred to as the “roots”.

The term “plant tissue” refers to any part of a plant, such as a plant organ. Examples of plant organs include, but are not limited to the leaf, stem, root, tuber, seed, branch, pubescence, nodule, leaf axil, flower, pollen, stamen, pistil, petal, peduncle, stalk, stigma, style, bract, fruit, trunk, carpel, sepal, anther, ovule, pedicel, needle, cone, rhizome, stolon, shoot, pericarp, endosperm, placenta, berry, stamen, and leaf sheath.

The term “seed” is meant to encompass the whole seed and/or all seed components, including, for example, the coleoptile and leaves, radicle and coleorhiza, scutellum, starchy endosperm, aleurone layer, pericarp and/or testa, either during seed maturation and seed germination.

The term “transgenic plant” means a plant that has been transformed with one or more exogenous nucleic acids. “Transformation” refers to a process by which a nucleic acid is integrated into the genome of a plant cell. “Stably integrated” refers to the permanent, or non-transient retention and/or expression of a polynucleotide in and by a cell genome. Thus, a stably integrated polynucleotide is one that is a fixture within a transformed cell genome and can be replicated and propagated through successive progeny of the cell or resultant transformed plant. Transformation may occur under natural or artificial conditions using various methods well known in the art. Transformation may rely on any known method for the insertion of nucleic acid sequences into a prokaryotic or eukaryotic host cell, including Agrobacterium-mediated transformation protocols, viral infection, whiskers, electroporation, heat shock, lipofection, polyethylene glycol treatment, micro-injection, and particle bombardment.

As used herein, the terms “stably expressed” or “stable expression” when used in reference to a protein refer to expression and accumulation of a protein in a host cell, such as a plant cell. In some embodiments, a protein may accumulate in a cell because it is not degraded by endogenous host cell proteases. In some embodiments, a protein is considered to be stably expressed in a plant if it is present in the plant in an amount of 1% or higher per total protein weight of soluble protein extractable from the plant.

The term “recombinant” refers to nucleic acids or proteins formed by laboratory methods of genetic recombination (e.g., molecular cloning) to bring together genetic material from multiple sources, creating sequences that would not otherwise be found in the genome. A recombinant fusion protein is a protein created by combining sequences encoding two or more constituent proteins, such that they are expressed as a single polypeptide. Recombinant fusion proteins may be expressed in vivo in various types of host cells, including plant cells, bacterial cells, fungal cells, mammalian cells, etc. Recombinant fusion proteins may also be generated in vitro.

The term “promoter” or a “transcription regulatory region” refers to nucleic acid sequences that influence and/or promote initiation of transcription. Promoters are typically considered to include regulatory regions, such as enhancer or inducer elements. The promoter will generally be appropriate to the host cell in which the target gene is being expressed. The promoter, together with other transcriptional and translational regulatory nucleic acid sequences (also termed “control sequences”), is necessary to express any given gene. In general, the transcriptional and translational regulatory sequences include, but are not limited to, promoter sequences, ribosomal binding sites, transcriptional start and stop sequences, translational start and stop sequences, and enhancer or activator sequences.

The term signal peptide—also known as “signal sequence”, “targeting signal”, “localization signal”, “localization sequence”, “transit peptide”, “leader sequence”, or “leader peptide”, is used herein to refer to an N-terminal peptide which directs a newly synthesized protein to a specific cellular location or pathway. Signal peptides are often cleaved from a protein during translation or transport and are therefore not typically present in a mature protein.

The term “proteolysis” or “proteolytic” or “proteolyze” means the breakdown of proteins into smaller polypeptides or amino acids. Uncatalyzed hydrolysis of peptide bonds is extremely slow. Proteolysis is typically catalyzed by cellular enzymes called proteases but may also occur by intra-molecular digestion. Low pH or high temperatures can also cause proteolysis non-enzymatically. Limited proteolysis of a polypeptide during or after translation in protein synthesis often occurs for many proteins. This may involve removal of the N-terminal methionine, signal peptide, and/or the conversion of an inactive or non-functional protein to an active one.

The term “purifying” is used interchangeably with the term “isolating” and generally refers to the separation of a particular component from other components of the environment in which it was found or produced. For example, purifying a recombinant protein from plant cells in which it was produced typically means subjecting transgenic protein containing plant material to biochemical purification and/or column chromatography.

When referring to expression of a protein in a specific amount per the total protein weight of the soluble protein extractable from the plant (“TSP”), it is meant an amount of a protein of interest relative to the total amount of protein that may reasonably be extracted from a plant using standard methods. Methods for extracting total protein from a plant are known in the art. For example, total protein may be extracted from seeds by bead beating seeds at about 15000 rpm for about 1 min. The resulting powder may then be resuspended in an appropriate buffer (e.g., 50 mM Carbonate-Bicarbonate pH 10.8, 1 mM DTT, 1× Protease Inhibitor Cocktail). After the resuspended powder is incubated at about 4° C. for about 15 minutes, the supernatant may be collected after centrifuging (e.g., at 4000 g, 20 min, 4° C.). Total protein may be measured using standard assays, such as a Bradford assay. The amount of protein of interest may be measured using methods known in the art, such as an ELISA or a Western Blot.

When referring to a nucleic acid sequence or protein sequence, the term “identity” is used to denote similarity between two sequences. Unless otherwise indicated, percent identities described herein are determined using the BLAST algorithm available at the world wide web address: blast.ncbi.nlm.nih.gov/Blast.cgi using default parameters.

As used herein, the terms “dicot” or “dicotyledon” or “dicotyledonous” refer to a flowering plant whose embryos have two seed leaves or cotyledons. Examples of dicots include, but are not limited to, Arabidopsis, tobacco, tomato, potato, sweet potato, cassava, alfalfa, lima bean, pea, chickpea, soybean, carrot, strawberry, lettuce, oak, maple, walnut, rose, mint, squash, daisy, Quinoa, buckwheat, mung bean, cow pea, lentil, lupin, peanut, fava bean, French beans (i.e., common beans), mustard, or cactus.

The terms “monocot” or “monocotyledon” or “monocotyledonous” refer to a flowering plant whose embryos have one cotyledon or seed leaf. Examples of monocots include, but are not limited to turf grass, maize (corn), rice, oat, wheat, barley, sorghum, orchid, iris, lily, onion, palm, and duckweed.

As used herein, a “low lactose product” is any food composition considered by the FDA to be “lactose reduced”, “low lactose”, or “lactose free”.

As used herein, a “milk protein” is any protein, or fragment or variant thereof, that is typically found in one or more mammalian milks.

As used herein, a “non-milk” protein is any protein that is not typically found in any mammalian milk. One non-limiting example of a non-milk protein is green fluorescent protein (GFP).

As used herein, an “exogenous intron” refers to an intronic sequence, or portion thereof, derived from a first cell type that is introduced into a second cell type. Thus, exogenous introns are not native to a host cell and/or host plant. Exogenous introns may, in some embodiments, comprise synthetic sequences and chimeric sequences. Exogenous introns do not typically code for amino acids, and are removed (i.e., spliced) by the host cell during translation of a protein from the transgene.

As used herein, a “nucleic acid” refers to a physical nucleic acid chemical structure. A nucleic acid is a polymeric form of nucleotides of any length, either deoxyribonucleotides or ribonucleotides or analogs thereof. The term also refers to both double and single stranded nucleic acid molecules. The following are non-limiting examples of a nucleic acid: a gene or gene fragment (for example, a probe, primer), an exon, an intron, intergenic DNA (including, without limitation, heterochromatic DNA), messenger RNA (mRNA), transfer RNA (tRNA), ribosomal RNA (rRNA), a ribozyme, cDNA, a recombinant polynucleotide, a branched polynucleotide, a plasmid, a vector, isolated DNA of a sequence, isolated RNA of a sequence, sgRNA, guide RNA, a nucleic acid probe, a primer, an snRNA, a long non-coding RNA, a snoRNA, a siRNA, a miRNA, a tRNA-derived small RNA (tsRNA), an antisense RNA, an shRNA, or a small rDNA-derived RNA (srRNA). A nucleic acid can comprise modified nucleotides, such as methylated nucleotides and nucleotide analogs. If present, modifications to the nucleotide structure can be imparted before or after assembly of the nucleic acid. The sequence of nucleotides can be interrupted by non-nucleotide components. A nucleic acid can be further modified after polymerization, such as by conjugation with a labeling component. A nucleic acid can also have secondary or tertiary structure. A nucleic acid can base pair, such as by way of Watson Crick base pairing.

As used herein, a “nucleic acid sequence” refers to a non-physical succession of bases indicating the order of nucleotides of a nucleic acid. A nucleic acid sequence is observable in written form such as on a machine (in silico) or handwritten. A nucleic acid sequence can be input or obtained from databases in a computer having a central processing unit

As used herein, the term “machine learning” refers to use of mathematical algorithms/models and related software that leverage data to improve performance of a task (e.g., predictions). Machine learning encompasses learning models capable of supervised learning, unsupervised learning, semi-supervised learning, and reinforcement learning. In some embodiments, machine learning utilizes alignment data and/or empirical data regarding RNA secondary or tertiary structure to improve RNA folding predictions or RNA structural comparisons. In a non-limiting example, the predictions made by machine learning algorithms and models of the present disclosure can inform selection of nucleic acid sequences for manufacture of a nucleic acid.

### Nucleic Acid Sequence Selection Based on Predicted Structures

In some embodiments, the present disclosure teaches methods for selecting a nucleic acid sequence based on differences in the predicted or empirically-determined structures of those sequences. Specifically, the present disclosure teaches a method of selecting nucleic acid sequences that result in similar predicted secondary structures from different RNA folding models. This invention is based in part, on the inventor's discovery that nucleic acid sequences that produce more similar predicted secondary structures across different RNA folding models, result in higher or more stable expression/accumulation of nucleic acids in vivo compared to nucleic acid sequences that produce more dissimilar predicted secondary structures.

Without wishing to be bound by any one theory, it is hypothesized that differences in predicted secondary structure produced from different RNA folding models are associated with decreased structural stability of the manufactured nucleic acid in vivo. That is, it is hypothesized that the differences in predicted structures from different models is indicative or suggestive that the sequence may take on different- or multiple-structures, when expressed in vivo, which may have deleterious effects on nucleic acid expression or accumulation in vivo.

In some embodiments, the present disclosure teaches a method comprising the steps of a) providing a plurality of nucleic acid sequences for evaluation; b) predicting secondary structure of the plurality of nucleic acid sequences, with a plurality of RNA folding models, such that each nucleic acid sequence in the plurality of nucleic acid sequences is associated with at least two predicted secondary structures; c) assessing structural similarity for the at least two predicted secondary structures associated with each nucleic acid sequence (e.g., via assignment of a structural similarity score); and d) selecting a nucleic acid sequence with higher structural similarity between predicted secondary structures than at least one other nucleic acid sequence in the plurality of nucleic acid sequences. In some embodiments, the selected nucleic acid sequence with higher similarity in its predicted secondary structures is predicted to express or accumulate at higher levels when expressed in vivo. In some embodiments, the predicted secondary structures are provided, and need not be predicted. The various aspects of the presently-disclosed invention are discussed in more detail, below.

### Plurality of Nucleic Acid Sequences

The present disclosure provides techniques for selecting a nucleic acid sequence from amongst a plurality of nucleic acid sequences. In some embodiments, the methods of the present disclosures are most effective when the selection is made within a group of related nucleic acid sequences. In some embodiments, it is hypothesized that selection of related nucleic acid sequences permits for selection based on predicted structure, while reducing potential confounding effects related to non-structural issues, such as the presence of RNAi targets, binding, or shuttling of nucleic acids in vivo, or other potential expression regulatory controls that may vary between highly disparate sequences. Thus, in some embodiments, the techniques of the present disclosure are more effective (i.e. are expected to produce the most accurate expression predictions), when applied to related sequences, including but not limited to: nucleic acid variants encoding the same or similar amino acid sequence (e.g., codon variants, or other sequence variations that in non-coding regions) or comprising other nucleic acid sequence variations that encode for amino acid chains that are at least 90%, 91%, 92%, 93%, 94%, 95%, 96%, 97%, 98%, or 99% sequence identity.

In some embodiments, the plurality of nucleic acid sequences are codon variants encoding the same or similar amino acid sequence. In some embodiments, the plurality of sequences encode for nucleic acids comprising RNAi hairpins, wherein the sequences vary in nucleic acids, but continue to be processed into small RNAs capable of interacting with the same target nucleotide. In some embodiments, the plurality of nucleic acid sequences are sequence other sequence variants that do not exhibit a biological function. For example, in some embodiments, the presently disclosed techniques can be applied to nucleic acid sequences encoding nucleic acids with laboratory applications, including probes, primers, linkers, bar codes, etc. In some embodiments the plurality of nucleic acid sequences are variants of riboswitches, aptamers, rRNAs or other non-coding RNAs.

In some embodiments, the plurality of nucleic acid sequences can be from any source, including, without limitation, randomly generated sequences, sequences derived from natural diversity (e.g., related sequences from related species), sequence rearrangements, artificial sequences, mutational library sequences, etc. Disclosure related to various types of plurality of nucleic acids is provided in this document, and is also known to those with skill in the art.

### Secondary Structures

Provided are nucleic acids with secondary structure. RNA secondary structure is represented by a sequence of bases, paired by hydrogen bonding, within its nucleotide sequence. Stacking these base pairs forms the scaffold driving the folding of RNA three-dimensional structures. As a result, the knowledge of the RNA secondary structure is useful for modeling RNA structures and understanding any functional mechanisms. The present disclosure provides in silico and wet lab approaches to determining secondary structures.

### RNA Folding Models

The present disclosure provides methods for predicting nucleic acid expression based on the similarity of secondary (or tertiary structures) developed from different RNA folding models. In some embodiments, the methods of the present disclosure are compatible with any RNA folding model. Persons having skill in the art are familiar with a variety of RNA folding models.

In some embodiments the RNA folding model utilizes comparative sequence analysis. Comparative sequence analysis is considered by some to be the most accurate computational method for determining the RNA secondary structure. This method assumes that the RNA secondary structure is evolutionarily conserved to a greater extent than the RNA sequence. This method usually finds the base pairs that covary to maintain Watson-Crick and wobble base pairs (compensatory mutations) of a given sequence using a set of homologous sequences. In some embodiments, comparative sequence analysis can be combined with score-based methods (See e.g., RNAalifold-Hofacker I L, Fekete M, Flamm C, Huynen M A, Rauscher S, Stolorz P E, et al. Automatic detection of conserved RNA structure elements in complete RNA virus genomes. Nucleic Acids Res. 1998; 26 (16):3825-36; KnetFold-Bindewald E, Shapiro B A. RNA secondary structure prediction from sequence alignments using a network of k-nearest neighbor classifiers. RNA. 2006; 12(3):342-52; and IL-Ruan J, Stormo G D, Zhang W. An iterated loop matching approach to the prediction of RNA secondary structures with pseudoknots. Bioinformatics. 2004; 20(1):58-66).

Other RNA folding models are based on score assignment, where only a single RNA sequence is required as the input. These methods assume that the native RNA structure is a structure with a minimum/maximum total score, depending on the hypothesis of RNA folding mechanism or its simplification. Hence, the problem of RNA secondary structure prediction is transformed into an optimization problem. Since the RNA secondary structure can be recursively broken down into smaller elements with independent score contributions, the dynamic programming (DP) algorithm is often employed to identify the optimal structure. Evaluation of the score for structure elements requires a score scheme of many parameter.

In some embodiments, any RNA folding model can be utilized in a method of the disclosure. Exemplary, non-limiting methods are provided in Table 1.

TABLE 1
Exemplary RNA Folding Models

| Name | Description | References |
|---|---|---|
| CentroidFold | Secondary structure | Hamada M, Kiryu H, Sato K, |
| | prediction based on | Mituyama T, Asai K (February |
| | generalized centroid | 2009). “Prediction of RNA |
| | estimator | secondary structure using generalized |
| | | centroid estimators”. Bioinformatics. |
| | | 25 (4): 465-473. |
| | | doi: 10.1093/bioinformatics/btn601. |
| | | PMID 19095700. |
| CentroidHomfold | Secondary structure | Hamada M, Sato K, Kiryu H, |
| | prediction by using | Mituyama T, Asai K (June 2009). |
| | homologous sequence | “Predictions of RNA secondary |
| | information | structure by combining homologous |
| | | sequence information”. |
| | | Bioinformatics. 25 (12): i330-i338. |
| | | doi: 10.1093/bioinformatics/btp228. |
| | | PMC 2687982. PMID 19478007. |
| Context Fold | An RNA secondary | Zakov S, Goldberg Y, Elhadad M, |
| | structure prediction software | Ziv-Ukelson M (November 2011). |
| | based on feature-rich trained | “Rich parameterization improves |
| | scoring models. | RNA structure prediction”. Journal |
| | | of Computational Biology. 18 (11): |
| | | 1525-1542. |
| | | Bibcode: 2011LNCS.6577 . . . 546Z. |
| | | doi: 10.1089/cmb.2011.0184. PMID |
| | | 22035327. |
| CONTRAfold | Secondary structure | Do C B, Woods D A, Batzoglou S |
| | prediction method based on | (July 2006). “CONTRAfold: RNA |
| | conditional log-linear | secondary structure prediction |
| | models (CLLMs), a flexible | without physics-based models”. |
| | class of probabilistic models | Bioinformatics. 22 (14): e90-e98. |
| | which generalize | doi: 10.1093/bioinformatics/btl246. |
| | upon SCFGs by using | PMID 16873527. |
| | discriminative training | |
| | and feature-rich scoring. | |
| Crumple | Simple, cleanly written | Schroeder S J, Stone J W, Bleckley S, |
| | software to produce the full | Gibbons T, Mathews D M (July 2011). |
| | set of possible secondary | “Ensemble of secondary structures |
| | structures for one sequence, | for encapsidated satellite |
| | given optional constraints. | tobacco mosaic virus RNA consistent |
| | | with chemical probing and |
| | | crystallography constraints”. |
| | | Biophysical Journal. 101 (1): 167- |
| | | 175. Bibcode: 2011BpJ . . . 101 . . . 167S. |
| | | doi: 10.1016/j.bpj.2011.05.053. PMC |
| | | 3127170. PMID 21723827. |
| CyloFold | Secondary structure | Bindewald E, Kluth T, Shapiro B A |
| | prediction method based on | (July 2010). “CyloFold: secondary |
| | placement of helices | structure prediction including |
| | allowing complex | pseudoknots”. Nucleic Acids |
| | pseudoknots. | Research. 38 (Web Server issue): |
| | | W368-W372. |
| | | doi: 10.1093/nar/gkq432. PMC |
| | | 2896150. PMID 20501603. |
| E2Efold | A deep learning based | Chen X, Li Y, Umarov R, Gao X, |
| | method for efficiently | Song L (2020). “RNA Secondary |
| | predicting secondary | Structure Prediction By Learning |
| | structure by differentiating | Unrolled Algorithms”. |
| | through a constrained | arXiv: 2002.05810 [cs.LG]. |
| | optimization solver, without | |
| | using dynamic programming. | |
| GTFold | Fast and scalable multicore | Swenson M S, Anderson J, Ash A, |
| | code for predicting RNA | Gaurav P, Sükösd Z, Bader D A, et al. |
| | secondary structure. | (July 2012). “GTfold: enabling |
| | | parallel RNA secondary structure |
| | | prediction on multi-core desktops”. |
| | | BMC Research Notes. 5: 341. |
| | | doi: 10.1186/1756-0500-5-341. PMC |
| | | 3748833. PMID 22747589. |
| IPknot | Fast and accurate prediction | Sato K, Kato Y, Hamada M, Akutsu |
| | of RNA secondary | T, Asai K (July 2011). “IPknot: fast |
| | structures with pseudoknots | and accurate prediction of RNA |
| | using integer programming. | secondary structures with |
| | | pseudoknots using integer |
| | | programming”. Bioinformatics. 27 |
| | | (13): i85-i93. |
| | | doi: 10.1093/bioinformatics/btr215. |
| | | PMC 3117384. PMID 21685106. |
| KineFold | Folding kinetics of RNA | Xayaphoummine A, Bucher T, |
| | sequences including | Isambert H (July 2005). “Kinefold |
| | pseudoknots by including an | web server for RNA/DNA folding |
| | implementation of the | path and structure prediction |
| | partition function for knots. | including pseudoknots and knots”. |
| | | Nucleic Acids Research. 33 (Web |
| | | Server issue): W605-W610. |
| | | doi: 10.1093/nar/gki447. PMC |
| | | 1160208. PMID 15980546. |
| Mfold | (Minimum Free Energy) | Zuker M, Stiegler P (January 1981). |
| | RNA structure prediction | “Optimal computer folding of large |
| | algorithm. | RNA sequences using |
| | | thermodynamics and auxiliary |
| | | information”. Nucleic Acids |
| | | Research. 9 (1): 133-148. |
| | | doi: 10.1093/nar/9.1.133. PMC |
| | | 326673. PMID 6163133. |
| pKiss | A dynamic programming | Theis, Corinna and Janssen, Stefan |
| | algorithm for the prediction | and Giegerich, Robert (2010). |
| | of a restricted class (H-type | “Prediction of RNA Secondary |
| | and kissing hairpins) of | Structure Including Kissing Hairpin |
| | RNA pseudoknots. | Motifs”. In Moulton, Vincent and |
| | | Singh, Mona (ed.). Algorithms in |
| | | Bioinformatics. Vol. 6293 (Lecture |
| | | Notes in Computer Science ed.). |
| | | Springer Berlin Heidelberg. pp. 52- |
| | | 64. doi: 10.1007/978-3-642-15294- |
| | | 8_5. ISBN 978-3-642-15293-1. |
| Pknots | A dynamic programming | Rivas E, Eddy S R (February 1999). |
| | algorithm for optimal RNA | “A dynamic programming algorithm |
| | pseudoknot prediction using | for RNA structure prediction |
| | the nearest neighbour energy | including pseudoknots”. Journal of |
| | model. | Molecular Biology. 285 (5): 2053- |
| | | 2068. arXiv: physics/9807048. |
| | | doi: 10.1006/jmbi. 1998.2436. PMID |
| | | 9925784. S2CID 2228845. |
| PknotsRG | A dynamic programming | Reeder J, Steffen P, Giegerich R |
| | algorithm for the prediction | (July 2007). “pknotsRG: RNA |
| | of a restricted class (H-type) | pseudoknot folding including near- |
| | of RNA pseudoknots. | optimal structures and sliding |
| | | windows”. Nucleic Acids Research. |
| | | 35 (Web Server issue): W320-W324. |
| | | doi: 10.1093/nar/gkm258. PMC |
| | | 1933184. PMID 17478505. |
| RNA123 | Secondary structure | RNA123 |
| | prediction via | |
| | thermodynamic-based | |
| | folding algorithms and novel | |
| | structure-based sequence | |
| | alignment specific for RNA. | |
| RNAfold | MFE RNA structure | I. L. Hofacker; W. Fontana; P. F. |
| | prediction algorithm. | Stadler; S. Bonhoeffer; M. Tacker; P. |
| | Includes an implementation | Schuster (1994). “Fast Folding and |
| | of the partition function for | Comparison of RNA Secondary |
| | computing basepair | Structures”. Monatshefte fur Chemie. |
| | probabilities and circular | 125 (2): 167-188. |
| | RNA folding. | doi: 10.1007/BF00818163. S2CID |
| | | 19344304. |
| RNAshapes | MFE RNA structure | Giegerich R, Voss B, Rehmsmeier M |
| | prediction based on abstract | (2004). “Abstract shapes of RNA”. |
| | shapes. Shape abstraction | Nucleic Acids Research. 32 (16): |
| | retains adjacency and | 4843-4851. doi: 10.1093/nar/gkh779. |
| | nesting of structural | PMC 519098. PMID 15371549. |
| | features, but disregards helix | |
| | lengths, thus reduces the | |
| | number of suboptimal | |
| | solutions without losing | |
| | significant information. | |
| | Furthermore, shapes | |
| | represent classes of | |
| | structures for which | |
| | probabilities based on | |
| | Boltzmann-weighted | |
| | energies can be computed. | |
| RNAstructure | A program to predict lowest | Mathews D H, Disney M D, Childs |
| | free energy structures and | J L, Schroeder S J, Zuker M, Turner |
| | base pair probabilities for | D H (May 2004). “Incorporating |
| | RNA or DNA sequences. | chemical modification constraints |
| | Programs are also available | into a dynamic programming |
| | to predict maximum | algorithm for prediction of RNA |
| | expected accuracy structures | secondary structure”. Proceedings of |
| | and these can include | the National Academy of Sciences of |
| | pseudoknots. Structure | the United States of America. 101 |
| | prediction can be | (19): 7287-7292. |
| | constrained using | Bibcode: 2004PNAS . . . 101.7287M. |
| | experimental data, including | doi: 10.1073/pnas.0401799101. PMC |
| | SHAPE, enzymatic | 409911. PMID 15123812. |
| | cleavage, and chemical | |
| | modification accessibility. | |
| | Graphical user interfaces are | |
| | available for Windows, Mac | |
| | OS X, Linux. Programs are | |
| | also available for use with | |
| | Unix-style text interfaces. | |
| | Also, a C++ class library is | |
| | available. | |
| SARNA-Predict | RNA Secondary structure | Tsang H H, Wiese K C (2010). |
| | prediction method based on | “SARNA-Predict: accuracy |
| | simulated annealing. It can | improvement of RNA secondary |
| | also predict structure with | structure prediction using |
| | pseudoknots. | permutation-based simulated |
| | | annealing”. IEEE/ACM Transactions |
| | | on Computational Biology and |
| | | Bioinformatics. 7 (4): 727-740. |
| | | doi: 10.1109/TCBB.2008.97. PMID |
| | | 21030739. S2CID 12095376. |
| seqfold | Predict the minimum free | seqfold, Lattice Automation, 2022 |
| | energy structure of nucleic | Mar. 27, retrieved 2022 Mar. 27 |
| | acids. seqfold is an | |
| | implementation of the | |
| | Zuker, 1981 dynamic | |
| | programming algorithm, the | |
| | basis for UNAFold/mfold, | |
| | with energy functions from | |
| | SantaLucia, 2004 (DNA) | |
| | and Turner, 2009 (RNA). | |
| | MIT license. Python CLI or | |
| | module. | |
| Sfold | Statistical sampling of all | Ding Y, Lawrence C E (December |
| | possible structures. The | 2003). “A statistical sampling |
| | sampling is weighted by | algorithm for RNA secondary |
| | partition function | structure prediction”. Nucleic Acids |
| | probabilities. | Research. 31 (24): 7280-7301. |
| | | doi: 10.1093/nar/gkg938. PMC |
| | | 297010. PMID 14654704. |
| Sliding Windows & | Sliding windows and | Schroeder S J, Stone J W, Bleckley S, |
| Assembly | assembly is a tool chain for | Gibbons T, Mathews D M (July |
| | folding long series of similar | 2011). “Ensemble of secondary |
| | hairpins. | structures for encapsidated satellite |
| | | tobacco mosaic virus RNA consistent |
| | | with chemical probing and |
| | | crystallography constraints”. |
| | | Biophysical Journal. 101 (1): 167- |
| | | 175. Bibcode: 2011BpJ . . . 101 . . . 167S. |
| | | doi: 10.1016/j.bpj.2011.05.053. PMC |
| | | 3127170. PMID 21723827. |
| SPOT-RNA | SPOT-RNA is first RNA | Singh J, Hanson J, Paliwal K, Zhou |
| | secondary structure | Y (November 2019). “RNA |
| | predictor which can predict | secondary structure prediction using |
| | all kind base pairs | an ensemble of two-dimensional |
| | (canonical, noncanonical, | deep neural networks and transfer |
| | pseudoknots, and base | learning”. Nature Communications. |
| | triplets). | 10 (1): 5407. |
| | | Bibcode: 2019NatCo . . . 10.5407S. |
| | | doi: 10.1038/s41467-019-13395-9. |
| | | PMC 6881452. PMID 31776342. |
| SwiSpot | Command-line utility for | Barsacchi M, Novoa E M, Kellis M, |
| | predicting alternative | Bechini A (November 2016). |
| | (secondary) configurations | “SwiSpot: modeling riboswitches by |
| | of riboswitches. It is based | spotting out switching sequences”. |
| | on the prediction of the so- | Bioinformatics. 32 (21): 3252-3259. |
| | called switching sequence, | doi: 10.1093/bioinformatics/btw401. |
| | to subsequently constrain | PMID 27378291. |
| | the folding of the two | |
| | functional structures. | |
| UNAFold | The UNAFold software | Markham N R, Zuker M (2008). |
| | package is an integrated | UNAFold: software for nucleic acid |
| | collection of programs that | folding and hybridization. Methods |
| | simulate folding, | in Molecular Biology. Vol. 453. pp. |
| | hybridization, and melting | 3-31. doi: 10.1007/978-1-60327-429- |
| | pathways for one or two | 6_1. ISBN 978-1-60327-428-9. |
| | single-stranded nucleic acid | PMID 18712296. |
| | sequences. | |
| vsfold/vs subopt | Folds and predicts RNA | Dawson W K, Fujiwara K, Kawai G |
| | secondary structure and | (September 2007). “Prediction of |
| | pseudoknots using an | RNA pseudoknots using heuristic |
| | entropy model derived from | modeling with mapping and |
| | polymer physics. The | sequential folding”. PLOS ONE. 2 |
| | program vs_subopt | (9): e905. |
| | computes suboptimal | Bibcode: 2007PLoSO . . . 2 . . . 905D. |
| | structures based on the free | doi: 10.1371/journal.pone.0000905. |
| | energy landscape derived | PMC 1975678. PMID 17878940. |
| | from vsfold5. | |
| Cocke-Younger Kasami | It employs bottom-up | Walter, H. K., Brandt, U. (2000). The |
| | parsing and dynamic | Cocke-Younger-Kasami |
| | programming to predict | Algorithm. Germany: Techn. Univ., |
| | structure. | Fachbereich Informatik. |
| loop-based energy | Determines RNAs having | Mathews, David H et al. “Folding |
| | more favorable folding by | and finding RNA secondary |
| | way of free energies. | structure.” Cold Spring Harbor |
| | | perspectives in biology vol. 2, 12 |
| | | (2010): a003665. |
| | | doi: 10.1101/cshperspect.a003665 |

In some embodiments, an RNA folding model is selected from (and/or is contained within the software identified in) Table 1. In some embodiments, an RNA folding model comprises a model selected from the group consisting of Cocke-Younger Kasami model, inside and outside model, loop-based energy model, minimum free energy, centroid, CONTRAfold, CentroidFold, ContextFold, and combinations thereof. In some embodiments, an RNA structure is determined by a model selected from the group consisting of minimum free energy, centroid (e.g., centroidFold), suboptimal folding, and any combination thereof.

In some embodiments, a nearest-neighbor (NN) model, and variants derived therefrom, is utilized to predict RNA structure. The NN model can be used for the calculation of energy changes of any structure of a given RNA molecule, and the DP algorithm can be also employed to efficiently find the MFE structure. For predicting a structure with noncanonical base pairs, some other score schemes can be employed as scoring functions, such as nucleotide cyclic motifs score system or equilibrium partition function. In an exemplary approach for RNA secondary-structure prediction, a single RNA sequence is folded according to an appropriate scoring function. In this approach, RNA structure can be divided into substructures such as loops and stems according to the nearest-neighbor model. Dynamic programming algorithms can then be employed for locating the global minimum or probabilistic structures from these substructures. The scoring parameters of each substructure can be obtained experimentally (e.g., RNAfold, RNAstructure, and RNAshapes) or by machine learning (e.g., CONTRAfold, CentroidFold, ContextFold, and the like). In some embodiments, RNAfold is utilized. In some embodiments, CentroidFold is utilized.

In some embodiments, RNA expression can be associated with predicted secondary or tertiary structures across multiple RNA folding models. In some embodiments, methods disclosed herein comprise use of one or more models. In some embodiments, methods disclosed herein comprise use of two or more models. In some embodiments, from about 0, 1, 2, 3, 4, or 5 models are employed.

In some embodiments, nucleic acid sequences having increased RNA and/or protein expression comprise similar or identical predicted secondary or tertiary structure across two or more models. In some embodiments, nucleic acid sequences comprising a codon variation comprise increased RNA and/or protein expression. The nucleic acid sequences comprising the codon variation may also have similar or identical predicted secondary or tertiary structure across two or more models. Exemplary codon variations are provided herein and any of which can be employed to increase expression.

ML-based methods for RNA secondary structure prediction can generally be divided into 3 categories according to the subprocess that ML participates in, i.e., score scheme based on ML, preprocessing and postprocessing based on ML, and prediction process based on ML. In some embodiments, the ML-based models learn functions that map inputs (features) to outputs by adjusting model parameters based on the known input-output pairs. Many of them employ free energy parameters, encoded RNA sequences, sequence patterns, or evolutionary information as key features, and their outputs can be classification labels (such as paired or unpaired) or continuous values (such as free energy). When a new input is fed to the trained model, the model can classify a corresponding label or predict a corresponding value. A non-limiting list of ML RNA folding models is provided in Table 2, below. In some embodiments, an RNA folding model of Table 1 also employs machine learning.

TABLE 2
Non-Limiting List of ML-based RNA Secondary Structure Prediction Methods.

| Category | | ML Technique | Reference |
|---|---|---|---|
| Score scheme | Free energy | Linear regression | Xia T B, SantaLucia J, Burkard M E, Kierzek R, Schroeder |
| based on ML | parameter- | | S J, Jiao X Q, et al. Thermodynamic parameters for an |
| model | refining | | expanded nearest-neighbor model for formation of RNA |
| | approach | | duplexes with Watson-Crick base pairs. Biochemistry. |
| | based on ML | | 1998; 37(42): 14719-35. |
| | | Constraint generation | Andronescu M, Condon A, Hoos H H, Mathews D H, |
| | | | Murphy K P. Efficient parameter estimation for RNA |
| | | | secondary structure prediction. Bioinformatics. 2007; |
| | | | 23(13): i19-i28. |
| | | Loss-augmented | Andronescu M, Condon A, Hoos H H, Mathews D H, |
| | | max-margin | Murphy K P. Computational approaches for RNA energy |
| | | constraint generation | parameter estimation. RNA. 2010; 16(12): 2304-18. |
| | | model, Boltzmann- | |
| | | likelihood model | |
| | Weighted | Discriminative | Zakov S, Goldberg Y, Elhadad M, Ziv-Ukelson M. Rich |
| | approach | structured-prediction | parameterization improves RNA structure prediction. J |
| | based on ML | learning framework | Comput Biol. 2011; 18(11): 1525-42. |
| | | combined, online | |
| | | learning algorithm | |
| | | SSVM | Akiyama M, Sato K, Sakakibara Y. A max-margin |
| | | | training of RNA secondary structure prediction integrated |
| | | | with the thermodynamic model. J Bioinform Comput Biol. |
| | | | 2018; 16(6): 1840025. |
| | | Deep neural network | Sato K, Akiyama M, Sakakibara Y. RNA secondary |
| | | | structure prediction using deep learning with |
| | | | thermodynamic integration. Nat Commun. 2021; |
| | | | [symbol] |
| | Probabilistic | EM method | Sakakibara Y, Brown M, Hughey R, Mian I S, Sjölander |
| | approach | | K, Underwood R C, et al. Stochastic contextfree grammars |
| | based on ML | | for tRNA modeling. Nucleic Acids Res. 1994; |
| | | | [symbol] |
| | | EM method | Knudsen B, Hein J. RNA secondary structure prediction |
| | | | using stochastic context-free grammars and evolutionary |
| | | | history. Bioinformatics. 1999; 15(6): 446-54. |
| | | EM method | Knudsen B, Hein J. Pfold: RNA secondary structure |
| | | | prediction using stochastic context-free grammars. |
| | | | Nucleic Acids Res. 2003; 31(13): 3423-8. |
| | | CLLM | Do C B, Woods D A, Batzoglou S. CONTRAfold: RNA |
| | | | secondary structure prediction without physicsbased |
| | | | models. Bioinformatics. 2006; 22(14): e90-e8. |
| | | Semi-supervised | Yonemoto H, Asai K, Hamada M. A semi-supervised |
| | | learning algorithm | learning approach for RNA secondary structure |
| | | | prediction. Comput Biol Chem. 2015; 57: 72-9. |
| Preprocessing | Preprocessing | SVM | Hor C-Y, Yang C-B, Chang C-H, Tseng C-T, Chen H-H. |
| and | based on ML | | A Tool Preference Choice Method for RNA Secondary |
| postprocessing | model | | Structure Prediction by SVM with Statistical Tests. Evol |
| based on ML | | | Bioinformatics Online. 2013; 9: 163-84. |
| model | | Statistical context- | Zhu Y, Xie Z Y, Li Y Z, Zhu M, Chen Y P P. Research on |
| | | free grammar model | folding diversity in statistical learning methods for RNA |
| | | | secondary structure prediction. Int J Biol Sci. 2018; |
| | | | 14(8): 872-82. |
| | Postprocessing | MLP | Haynes T, Knisley D, Knisley J. Using a neural network |
| | based on ML | | to identify secondary RNA structures quantified by |
| | model | | graphical invariants. Match Commun Math Comput |
| | | | Chem. 2008; 60(2): 277-90. |
| | | MLP | Koessler D R, Knisley D J, Knisley J, Haynes T. A |
| | | | predictive model for secondary RNA structure using graph |
| | | | theory and a neural network. BMC Bioinformatics. 2010; |
| | | | 11(Suppl 6): S21. |
| Predicting | End-to-end | System composed | Takefuji Y, Chen L L, Lee K C, Huffman J. Parallel |
| process based | approach | of several | algorithms for finding a near-maximum independent set of |
| on ML model | | interactional | a circle graph. IEEE Trans Neural Netw. 1990; 1(3): 263- |
| | | neurons | 7. |
| | | Hopfield networks | Liu Q, Ye X, Zhang Y. A Hopfield Neural Network based |
| | | | algorithm for RNA secondary structure prediction. 1st |
| | | | International Multi Symposium on Computer and |
| | | | Computational Sciences; Hangzhou, China: IEEE; 2006. |
| | | MLP | Qasim R, Kauser N, Jilani T. Secondary Structure |
| | | | Prediction of RNA using Machine Learning Method. Int J |
| | | | Comput Appl. 2011; 10(6): 0975-8887. |
| | | MFT network | Steeg E W. Neural networks, adaptive optimization, and |
| | | | RNA secondary structure prediction. Artificial intelligence |
| | | | and molecular biology. 1993: 121-60. |
| | | MFT network with | Apolloni B, Lotorto L, Morpurgo A, Zanaboni A. RNA |
| | | mean field | Secondary Structure Prediction by MFT Neural Networks. |
| | | approximation to | Psychol Forsch. 2003: 143-8. |
| | | update network's | |
| | | nodes | |
| | | Compound deep | Singh J, Hanson J, Paliwal K, Zhou Y Q. SPOT-RNA: |
| | | neural networks, | RNA Secondary Structure Prediction using an Ensemble |
| | | transfer learning | of Two-dimensional Deep Neural Networks and Transfer |
| | | | Learning. Nat Commun. 2019; 10 (1): 1-13. |
| | | Compound deep | Chen X, Li Y, Umarov R, Gao X, Song L. RNA |
| | | neural networks | Secondary Structure Prediction By Learning Unrolled |
| | | | Algorithms. International Conference on Learning |
| | | | Representations. 2020. |
| | | CNN, MLP | Calonaci N, Jones A, Cuturello F, Sattler M, Bussi G. |
| | | | Machine learning a model for RNA structure prediction. |
| | | | 2020; 2(4): lqaa090. |
| | Hybrid | Hierarchical | Bindewald E, Shapiro B A. RNA secondary structure |
| | approach | network of k- | prediction from sequence alignments using a network of |
| | | nearest neighbor | k-nearest neighbor classifiers. RNA. 2006; 12(3): 342-52. |
| | | model | |
| | | Bi-LSTM | Quan L, Cai L, Chen Y, Mei J, Sun X, Lyu Q. Developing |
| | | | parallel ant colonies filtered by deep learned constrains for |
| | | | predicting RNA secondary structure with pseudo-knots. |
| | | | Neurocomputing. 2020; 384: 104-14. |
| | | Bi-LSTM | Wu H, Tang Y, Lu W, Chen C, Huang H, Fu Q, editors. |
| | | | RNA Secondary Structure Prediction Based on Long |
| | | | Short-Term Memory Model. 14th International |
| | | | Conference on Intelligent Computing (ICIC); 2018; |
| | | | Wuhan. China. |
| | | Bi-LSTM | Lu W, Tang Y, Wu H, Huang H, Fu Q, Qiu J, et al. |
| | | | Predicting RNA secondary structure via adaptive deep |
| | | | recurrent neural networks with energy-based filter. BMC |
| | | | Bioinformatics. 2019; 20(Suppl 25): 684. |
| | | CNN | Zhang H, Zhang C, Li Z, Li C, Wei X, Zhang B, et al. A |
| | | | New Method of RNA Secondary Structure Prediction |
| | | | Based on Convolutional Neural Network and Dynamic |
| | | | Programming. Front Genet. 2019; 10: 467. |
| | | Bi-LSTM | Wang L, Liu Y, Zhong X, Liu H, Lu C, Li C, et al. |
| | | | DMfold: A Novel Method to Predict RNA Secondary |
| | | | Structure With Pseudoknots Based on Deep Learning and |
| | | | Improved Base Pair Maximization Principle. Front Genet. |
| | | | 2019; 10: 143. |
| | | Bi-LSTM | Willmott D, Murrugarra D, Ye Q. Improving RNA |
| | | | secondary structure prediction via state inference with |
| | | | deep recurrent neural networks. Comput Math Biophys. |
| | | | 2020; 8: 36-50. |

CLLM, conditional log-linear model; CNN, convolutional neural network; EM, expectation-maximization; MFT, mean field theory; ML, machine learning; MLP, multilayer perceptron; SSVM, structured support vector machine; SVM, support vector machine.
Adapted from Zhao Q, Zhao Z, Fan X, Yuan Z, Mao Q, Yao Y (2021) Review of machine learning methods for RNA secondary structure prediction. PLOS Comput Biol 17(8): e1009291. [symbol]
indicates data missing or illegible when filed

### Wet-Lab Nucleic Acid Structures

In some embodiments, a wet lab method is utilized to predict or determine RNA structure. X-ray crystallography and nuclear magnetic resonance (NMR) are exemplary approaches for determining RNA structures, both of which can offer structural information at a single base pair resolution.

In some embodiments, a wet lab method comprises X-ray crystallography, see Westhof E. Twenty years of RNA crystallography. RNA. 2015; 21(4):486-7, included by reference herein in its entirety. In some embodiments, a wet lab method comprises NMR, see Westhof E. Twenty years of RNA crystallography. RNA. 2015; 21(4):486-7, herein incorporated by reference in its entirety.

In some embodiments, a method employing a wet-lab approach can precede a method comprising machine learning. In other words, structural predictions obtained from a wet-lab method can be input into a method employing machine learning. In some embodiments, images of a structure from a wet-lab analysis can be loaded onto a software of the disclose for nucleic acid structural comparison to inform a selection.

In some embodiments, the present disclosure teaches a modified method for nucleic acid structures determined via wet lab methods. Rather than reciting at least two predicted secondary structures from different RNA folding models, wet lab approaches seek to identify possible folding variants actually identified/observed using the wet lab technique. Thus in some embodiments, the step of predicting secondary structure is replaced with determining structure, and the step of determining structure similarity between predicted structures is replaced with determining structural similarity between actually observed structures.

### Assessing Similarity of Nucleic Acid Sequence Structures (Structural Similarity Scores)

The presently disclosed methods can employ any method for assessing similarity between secondary structures of a nucleic acid sequence. In some embodiments, the secondary structure comprises RNA structure. In some embodiments the secondary structure comprises single stranded DNA. In some embodiments, secondary structures of nucleic acids can be compared visually, or can be assessed entirely in silico. In some embodiments, the secondary structures are assessed via hybrid approaches. In some embodiments, similarity scores are saved/recorded/written down to permit further review/analysis. In some embodiments, similarity scores are assessed, but never recorded.

### In-Silico Similarity

In some embodiments, a comparison between predicted secondary or tertiary structures is determined. A comparison can employ in silico methods. In some embodiments, an in-silico method utilizes software that is configured to accept information (e.g., an image or other data file conveying information about a chemical structure of a nucleic acid or a nucleic acid sequence) and provide an output related to secondary structure and optionally a comparison of secondary structures of a plurality of nucleic acid sequences. Table 3 provides a summary of exemplary software that quantifies differences between nucleic acid sequences. Any of the software of Table 1 can also be utilized to analyze similarity of structures of nucleic acids.

TABLE 3
Exemplary software that quantifies differences. Additional
software is provided in Table 1, many of which are
also capable of structural comparisons.

| Software | Reference |
|---|---|
| RNAstructure | rna.urmc.rochester.edu/RNAstructureWeb/ |
| CoSSMos | Vanegas, P. L., Hudson, G. A., Davis, A. R., |
| | Kelly, S. C., Kirkpatrick, C. C., and Znosko, |
| | B. M. (2012) RNA CoSSMos: |
| | Characterization of Secondary Structure |
| | Motifs- a searchable database of secondary |
| | structure motifs in RNA three-dimensional |
| | structures, Nucleic Acids D439-D444. |
| RNAView | Yang, H., Jossinet, F., Leontis, N., Chen, L., |
| | Westbrook, J., Berman, H. M. and Westhof, |
| | E. (2003) Tools for the automatic |
| | identification and classification of RNA base |
| | pairs. Nucleic Acids Res, 31, 3450-3460 |

In some embodiments, structural similarity is determined by any other algorithmic/computational method known to persons having skill in the art, including those disclosed in Nikolova, N. and Jaworska, J. (2003), Approaches to Measure Chemical Similarity—a Review. QSAR Comb. Sci., 22: 1006-1026.

In some embodiments, structural similarity of the NP is evaluated by calculating the pairwise nucleic acid sequence secondary structure based on the Tanimoto coefficient and using the python library RDKit (www.rdkit.org). Briefly, morgan fingerprints are prepared for the at least two secondary structures. These fingerprints are then compared to assess similarity.

In some embodiments, the Tanimoto coefficient is calculated with the formula for dichotomous variables.

$${S}_{A B} = \frac{C}{A + B - C}$$

In some embodiments, the Tanimoto Coefficient is calculated using formula 1 for continuous variables.

$${S}_{A , B} = \frac{[ \sum nj = 1 XjAXjB ]}{[ \sum nj = 1 ( xjA ) 2 + \sum nj = 1 ( xjB ) 2 - \sum nj = 1 XjAXjB ]} \qquad Formula 1$$

Wherein the SAB similarity score between molecules A and B is calculated by dividing the “C” features in common between two structures, by the “A” the features of a first structure plus the “B” features of a second structure, minus C. That is, A is the number of on bits in molecule A, B is number of on bits in molecule B, while C is the number of bits that are on in both molecules. x_{jA} means the j-th feature of molecule A. x_{jB} means the j-th feature of molecule B. For more information on how to calculate the Tanimoto coefficient, see Bajusz, D., Rácz, A. & Héberger, K. Why is Tanimoto index an appropriate choice for fingerprint-based similarity calculations?. J Cheminform 7, 20 (2015).

In some embodiments Tanimoto coefficients range from 0 to 1 with 0 being no similarity and 1 being an identical molecule. In some embodiments. In some embodiments, two natural product structures are considered similar if they have a Tanimoto similarity coefficient of at least 0.6, 0.7, 0.8, 0.9, or 0.95, including all ranges and subranges therebetween.

### Visual (or Observed/Perceived) Similarity

In some embodiments, secondary structures of nucleic acid sequences are assessed visually or observationally. In some embodiments, the assessment results are quantitative. In some embodiments, the assessment is qualitative. The examples of the present disclosure identify a strong correlation between differences in secondary structures of the same nucleic acid sequence, and the resulting nucleic acid's expression/stability in vivo. Thus, in some embodiments, selecting a nucleic acid sequence with superior expression/stability can be done by picking out a sequence with secondary structures that are less different compared to the secondary structures of at least one other nucleic acid sequence. In some embodiments, this type of distinction can be achieved visually/observationally. For example, at least two secondary structures (e.g., a predicted minimum free energy structure and a predicted centroid structure) can be visually compared and optionally ranked according to perceived similarity. This visual analysis can be completed, for example, by stacking the predicted structure figures with about 50% translucency in a suitable computer program (e.g., Microsoft Word or the like), and visually assessing the amount of overlap. In some embodiments, the predicted structure figures are stacked with about 20%, 30%, 40%, 50%, 60%, 70%, 80%, or up to about 90% translucency, including all ranges and subranges therebetween. In some embodiments, the visual analysis can be conducted by comparing the structures side-by-side, such as on different columns of a table, or sequentially, such as in a flip book.

The visual assessment can be conducted purely on qualitative perception of differences, or can be done by counting number and size of different structures, such as number of loops, steps, helices, and the number of nucleic acids within them. In some embodiments the analysis can be done by assigning a score to each set of structures, or by instead ranking sets based on their similarity. Regardless of whether structure sets are assigned individual scores, or relative scores (e.g., ranking), these are considered a sequence similarity score within the context of this disclosure.

In some embodiments, a visual structure similarity analysis can be supplemented with an in silico analysis. For example, in some embodiments, the assessment of differences between two or more secondary structures can be conducted on the structure itself. In some embodiments, the assessment of differences can be conducted on a representation of structures. For example, in some embodiments, the secondary structures are assessed with the nucleotides represented in the structure. In some embodiments the secondary structures are assessed against wire models of the structures that do not identify individual nucleic acids. In some embodiments, the structures are assessed via shadow (e.g., silhouette) cutout representations of the space occupied by a structure. In some embodiments, structure similarity assessments can also be conducted on more abstract representations of structure. For example, in some embodiments, the structural similarity comparison can be conducted on mountain plot curves, representing the number of nucleic acid residues per position in a predicted structure (see discussion on mountain plots in the disclosure and also in Andreas R. Gruber, Ronny Lorenz, Stephan H. Bernhart, Richard Neuböck, Ivo L. Hofacker, The Vienna RNA Websuite, Nucleic Acids Research, Volume 36, Issue suppl_2, 1 Jul. 2008, Pages W70-W74).

### Similarity Variation of Wet-Lab Nucleic Acid Structures

In some embodiments, a wet-lab method is used to predict a structure of a nucleic acid sequence. Wet-lab methods such as X-ray crystallography and nuclear magnetic resonance (NMR) can offer structural information at a single base pair resolution. Although many methods have been developed to infer the state of nucleotides (paired or unpaired) in an RNA molecule using enzymatic or chemical probes coupled with next-generation sequencing most of them can only be used to capture the RNA secondary structure in vitro. The obtained structure may differ markedly from the in vivo conformation. Accordingly, wet-lab methods can be combined with at least one other structural prediction method disclosed herein. In some embodiments, a method comprises a wet-lab method (X-ray crystallography or NMR) and at least one other RNA structure prediction method (e.g., any from Table 1).

Structures obtained from wet-lab techniques can be evaluated in the same way as those developed from RNA folding models. In some embodiments differences in structures observed via wet lab techniques can be assessed visually (observationally) as described herein. In some embodiments differences in structures observed via wet lab techniques can be assessed in silico, using any known structure comparison strategy, including those described herein.

### Selection of Nucleic Acid Sequence

In some embodiments, the methods of the present disclosure recite selecting a nucleic acid sequence from amongst the plurality of nucleic acid sequences based on the similarity of the predicted secondary structure. In some embodiments, a nucleic acid sequence is selected if it has a structural similarity score that is higher (i.e., more similar) than at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more, other nucleic acid sequence(s) in the plurality of nucleic acid sequences. In some embodiments, a nucleic acid sequence is selected if it has the highest structural similarity score (i.e., most similar) than at least one other nucleic acid sequence in the plurality of nucleic acid sequences. In some embodiments, the selection is supervised or unsupervised. In some embodiments the selection is done in silico based on a set of pre-existing rules. In some embodiments, the selection is made by a user. As noted above, selection may utilize any appropriate scale for evaluating similarity. Exemplary means comprise, scoring, ranking, percent ranking, and combinations thereof.

In some embodiments, a selection can consider further elements beyond the similarity score. For example, in some embodiments, selection of the nucleic acid sequence can take into account presence or absence of a desired sequence, presence or absence of base pairing or base pairing potential, distance of predicted structural features (e.g., bulge, hairpin, internal loop), relaxed base-pair score, and combinations thereof. For example, in some embodiments, a nucleic acid sequence with a similarity score higher than at least one other nucleic acid sequence may further be evaluated/selected based on the presence of a desired loop length or the presence of other structures. In some embodiments, a nucleic acid sequence is selected due to the presence of an loops with optimal loop length of about 4-8 bp, and/or containing a tetraloop UUCG. In some embodiments a nucleic acid would be less likely to be selected if it exhibited unstable structures that would be expected to present pseudo-knots such as large loops with no secondary structure of their own and loops of less than 4 and more than 8 bp. Selection can also take into amount the frequency of any of the aforementioned aspects.

Scoring can comprise assigning a number from 0-1000. In some embodiments, a selection of a nucleic acid sequence is based on a lower number. In some embodiments a selection is based on a nucleic acid sequence having a higher number. In some embodiments, a sequence is selected having a score of about 0-5, 1-5, 1-10, 0-10, 5-15, 5-20, 15-30, 15-45, 0-50, 5-10, 1-20, 10-30, 15-75, and any subrange in between. In some embodiments, a sequence is selected having a score of about 50-200, 50-150, 100-200, 250-500, 300-500, or 500-1000, and any subrange in between.

In some embodiments, a selection comprises ranking a plurality of nucleic acid sequences according to their score. A selection can comprise selecting lower ranked nucleic acid sequences or higher ranked nucleic acid sequences. Percent ranking can also be employed. Percentile rank of a given score is the percentage of scores in its frequency distribution that are less than that score.

In some embodiments, selection of a nucleic acid sequence can be based on a nucleic acid sequence having a higher structural similarity score than at least one other nucleic acid sequence in a plurality of nucleic acid sequences. In some embodiments, methods comprised herein comprise an analysis comprising a mountain plot. Krouwer and Monti (1995) devised the mountain plot (also known as a folded empirical cumulative distribution plot) as a complementary representation of the difference plot. It shows the distribution of the differences with an emphasis on the center and the tails of the distribution. A mountain plot can be used to estimate the median of the differences, the central 95% interval, the range, and the percentage of observations outside the total allowable error bands. The mountain plot is a useful complementary plot to the Bland & Altman plot which can also be employed in any of the methods provided herein.

A structural similarity score can be increased by any amount. In some embodiments, a structural similarity score is increased by at least about or at most about 1-fold, 5-fold, 10-fold, 15-fold, 25-fold, 50-fold, 75-fold, 100-fold, 125-fold, 150-fold, 175-fold, 200-fold, 225-fold, 250-fold, 275-fold, 300-fold, 325-fold, 350-fold, 375-fold, 400-fold, 425-fold, 450-fold, 475-fold, or up to about 500-fold, including all ranges and subranges therebetween. In some embodiments, a structural similarity score is increased by at least about or at most about 5%, 10%, 20%, 30%, 40%, 50%, 60%, 70%, 80%, 90%, 100%, 110%, 120%, 130%, 140%, 150%, or up to about 200%, including all ranges and subranges therebetween.

In some embodiments, a method provided herein comprises determining a structural similarity score. A structural similarity score can be determined by way of any of the RNA structure prediction models provided herein.

In some embodiments, an in silico structural similarity analysis can inform selection of a nucleic acid sequence, from a plurality of nucleic acid sequences, to be manufactured into a nucleic acid. In some embodiments, provided methods comprise selecting a nucleic acid sequence with a higher structural similarity score than at least one other nucleic acid sequence in a plurality of nucleic acid sequences. In some embodiments, a selected nucleic acid sequence comprises an optimized codon sequence.

In some embodiments, manufactured nucleic acids can be evaluated for expression in host cells. In some embodiments, a structural similarity score obtained from an in silico analysis is plotted against empirical expression data obtained from manufacture. In some embodiments, nucleic acids showing high expression, comprise a logarithmic correlation between an in silico structural similarity analysis and a related empirical analysis. In some embodiments, a logarithmic correlation comprises an R2 correlation coefficient of at least about or at most about 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or up to about 1, including all ranges and subranges therebetween.

### Manufacturing

Provided herein are methods of manufacturing a nucleic acid. Persons having skill in the art will be familiar with the multiple strategies for manufacturing nucleic acids. In some embodiments, a selected nucleic acid sequence is manufactured into a nucleic acid. In some embodiments, the manufactured nucleic acid exhibits greater expression in a host cell as compared to a non-selected nucleic acid, if the non-selected nucleic acid were manufactured. In some embodiments, the increased expression is at least about or at most about: 0.5-fold, 1-fold, 3-fold, 5-fold, 7-fold, 10-fold, 12-fold, 15-fold, 20-fold, 30-fold, 40-fold, 50-fold, 60-fold, 70-fold, 80-fold, 90-fold, 100-fold, 110-fold, 120-fold, 130-fold, 140-fold, 150-fold, or up to about 200-fold, including all ranges and subranges therebetween.

In some embodiments, a nucleic acid comprises a sequence with at least about or at most about 85%, 90%, 95%, 96%, 97%, 98%, 99%, or 100% identity to any one of SEQ ID NO: 757, 760, 762, 763, 765, 772, 773, 778, and 780. In some embodiments, a nucleic acid comprises a sequence selected from the group consisting of: SEQ ID NO: 757, 760, 762, 763, 765, 772, 773, 778, and 780. In some embodiments, a nucleic acid comprises SEQ ID NO: 757. In some embodiments, a nucleic acid comprises SEQ ID NO: 760. In some embodiments, a nucleic acid comprises SEQ ID NO: 762. In some embodiments, a nucleic acid comprises SEQ ID NO: 763. In some embodiments, a nucleic acid comprises SEQ ID NO: 765. In some embodiments, a nucleic acid comprises SEQ ID NO: 772. In some embodiments, a nucleic acid comprises SEQ ID NO: 773. In some embodiments, a nucleic acid comprises SEQ ID NO: 778. In some embodiments, a nucleic acid comprises SEQ ID NO: 780. In some embodiments, a host cell comprises a nucleic acid encoding any one of SEQ ID NO: 685, 687, and 695. In some embodiments, a host cell comprises a nucleic acid encoding a sequence comprising at least about 85%, 90%, 95%, 96%, 97%, 98%, 99%, or 100% identity to any one of SEQ ID NO: 685, 687, and 695.

In some embodiments, a plurality of nucleic acid sequences that undergo a method to inform a selection encode for the same amino acid sequence (e.g., a protein with comparable identity and/or function). In some embodiments, the amino acid sequences comprise at least about or at most about 75%, 80%, 85%, 90%, 95%, 96%, 97%, 98%, 99%, or 100% sequence identity, including all ranges and subranges therebetween.

### Computer and Robotic System

In some embodiments, a method of the disclosure comprises a computer system and optionally a robotic system.

In addition, any or call of the methods of the disclosure can comprise automation, for example the systems may be at least partially automated or fully automated. In some embodiments, a system of the disclosure can comprise one or more work modules (e.g., a DNA/RNA synthesis module, a vector cloning module, a selection module, a sequencing module, and combinations thereof).

As will be appreciated by those in the art, an automated system can include a wide variety of components, including, but not limited to: liquid handlers; one or more robotic arms; plate handlers for the positioning of microplates; plate sealers, plate piercers, automated lid handlers to remove and replace lids for wells on non-cross contamination plates; disposable tip assemblies for sample distribution with disposable tips; washable tip assemblies for sample distribution; 96 well loading blocks; integrated thermal cyclers; cooled reagent racks; microtiter plate pipette positions (optionally cooled); stacking towers for plates and tips; magnetic bead processing stations; filtrations systems; plate shakers; barcode readers and applicators; and one or more computer systems. Provided systems can comprise use of a microtiter plate (e.g., a 96- or 384-well plate), optionally configured for automated systems.

In some embodiments, the robotic systems of the present disclosure comprise automated handling (e.g., a robotic arm) enabling high-throughput pipetting to perform any or all of the steps in a method described herein. Exemplary methods can comprise aspiration, dispensing, mixing, diluting, washing, accurate volumetric transfers; retrieving and discarding of pipette tips; and repetitive pipetting of identical volumes for multiple deliveries from a single sample aspiration. These manipulations can be cross-contamination free.

In some embodiments, the automated systems of the present disclosure are compatible with platforms for multi-well plates, deep-well plates, square well plates, reagent troughs, test tubes, mini tubes, microfuge tubes, cryovials, filters, micro array chips, optic fibers, beads, agarose and acrylamide gels, and other solid-phase matrices or platforms are accommodated on an upgradeable modular deck. In some embodiments, the automated systems of the present disclosure contain at least one modular deck for multi-position work surfaces for placing source and output samples, reagents, sample and reagent dilution, assay plates, sample and reagent reservoirs, pipette tips, and an active tip-washing station.

In some embodiments, an integrated thermal cycler and/or thermal regulators are used for stabilizing the temperature of heat exchangers such as controlled blocks or platforms to provide accurate temperature control of incubating samples from 0° C. to 100° C.

In some embodiments, an automated system of the present disclosure is designed to be flexible and adaptable with multiple hardware add-ons to allow the system to carry out multiple applications. In some embodiments, a software program module can allow creation, modification, and running of methods. A system's diagnostic modules can allow setup, instrument alignment, and motor operations. The customized tools, labware, and liquid and particle transfer patterns allow different applications to be programmed and performed. A database can allow method and parameter storage. Robotic and computer interfaces allow communication between instruments.

Persons having skill in the art will recognize the various robotic platforms capable of carrying out any of the methods of the present disclosure. Table 3.5 below provides a non-exclusive list of scientific equipment capable of carrying out steps of the disclosure.

TABLE 3.5
Exemplary equipment that can be comprised in a method of the disclosure.

| Equipment Type | Operation(s) performed | Compatible Equipment |
|---|---|---|
| liquid handlers | Hitpicking (combining | Hamilton Microlab STAR, |
| | by transferring) | Labcyte Echo 550, Tecan |
| | primers/templates for | EVO 200, Beckman Coulter |
| | PCR amplification of | Biomek FX, or equivalents |
| | DNA parts | |
| Thermal cyclers | PCR amplification of | Inheco Cycler, ABI 2720, ABI |
| | DNA parts | Proflex 384, ABI Veriti, or |
| | | equivalents |
| Fragment | gel electrophoresis to | Agilent Bioanalyzer, AATI |
| analyzers | confirm PCR products | Fragment Analyzer, or |
| (capillary | of appropriate size | equivalents |
| electrophoresis) | | |
| Sequencer | Verifying sequence of | Beckman Ceq-8000, Beckman |
| (sanger: Beckman) | parts/templates | GenomeLab ™, or equivalents |
| NGS (next | Verifying sequence of | Illumina MiSeq series |
| generation | parts/templates | sequences, illumina Hi-Seq, |
| sequencing) | | Ion torrent, pac bio or other |
| instrument | | equivalents |
| nanodrop/plate | assessing concentration | Molecular Devices |
| reader | of DNA samples | SpectraMax M5, Tecan |
| | | M1000, or equivalents. |
| liquid handlers | Hitpicking (combining by | Hamilton Microlab STAR, |
| | transferring) DNA parts | Labcyte Echo 550, Tecan |
| | for assembly along with | EVO 200, Beckman Coulter |
| | cloning vector, addition | Biomek FX, or equivalents |
| | of reagents for assembly | |
| | reaction/process | |
| Colony pickers | for inoculating colonies | Scirobotics Pickolo, |
| | in liquid media | Molecular Devices QPix 420 |
| liquid handlers | Hitpicking | Hamilton Microlab STAR, |
| | primers/templates, | Labcyte Echo 550, Tecan |
| | diluting samples | EVO 200, Beckman Coulter |
| | | Biomek FX, or equivalents |
| Fragment | gel electrophoresis to | Agilent Bioanalyzer, AATI |
| analyzers | confirm assembled | Fragment Analyzer |
| (capillary | products of appropriate | |
| electrophoresis) | size | |
| Sequencer | Verifying sequence of | ABI3730 Thermo Fisher, |
| (sanger: Beckman) | assembled plasmids | Beckman Ceq-8000, Beckman |
| | | GenomeLab ™, or equivalents |
| NGS (next | Verifying sequence of | Illumina MiSeq series |
| generation | assembled plasmids | sequences, illumina Hi-Seq, |
| sequencing) | | Ion torrent, pac bio or other |
| instrument | | equivalents |
| centrifuge | spinning/pelleting cells | Beckman Avanti floor |
| | | centrifuge, Hettich Centrifuge |
| Electroporators | electroporative | BTX Gemini X2, BIO-RAD |
| | transformation of cells | MicroPulser Electroporator |
| Ballistic | ballistic transformation of | BIO-RAD PDS1000 |
| transformation | cells | |
| Incubators, | for chemical | Inheco Cycler, ABI 2720, ABI |
| thermal cyclers | transformation/heat shock | Proflex 384, ABI Veriti, or |
| | | equivalents |
| Liquid handlers | for combining DNA, | Hamilton Microlab STAR, |
| | cells, buffer | Labcyte Echo 550, Tecan |
| | | EVO 200, Beckman Coulter |
| | | Biomek FX, or equivalents |
| Colony pickers | for inoculating colonies | Scirobotics Pickolo, |
| | in liquid media | Molecular Devices QPix 420 |
| Liquid handlers | For transferring cells | Hamilton Microlab STAR, |
| | onto Agar, transferring | Labcyte Echo 550, Tecan |
| | from culture plates to | EVO 200, Beckman Coulter |
| | different culture plates | Biomek FX, or equivalents |
| | (inoculation into other | |
| | selective media) | |
| Platform shaker- | incubation with shaking | Kuhner Shaker ISF4-X, |
| incubators | of microtiter plate cultures | Infors-ht Multitron Pro |
| Colony pickers | for inoculating colonies | Scirobotics Pickolo, |
| | in liquid media | Molecular Devices QPix 420 |
| liquid handlers | Hitpicking | Hamilton Microlab STAR, |
| | primers/templates, | Labcyte Echo 550, Tecan |
| | diluting samples | EVO 200, Beckman Coulter |
| | | Biomek FX, or equivalents |
| Thermal cyclers | cPCR verification of | Inheco Cycler, ABI 2720, ABI |
| | strains | Proflex 384, ABI Veriti, or |
| | | equivalents |
| Fragment | gel electrophoresis to | Infors-ht Multitron Pro, |
| analyzers | confirm cPCR products | Kuhner Shaker ISF4-X |
| (capillary | of appropriate size | |
| electrophoresis) | | |
| Sequencer | Sequence verification of | Beckman Ceq-8000, Beckman |
| (sanger: Beckman) | introduced modification | GenomeLab ™, or equivalents |
| NGS (next | Sequence verification of | Illumina MiSeq series |
| generation | introduced modification | sequences, illumina Hi-Seq, |
| sequencing) | | Ion torrent, pac bio or other |
| instrument | | equivalents |
| Liquid handlers | For transferring from | Hamilton Microlab STAR, |
| | culture plates to different | Labcyte Echo 550, Tecan |
| | culture plates | EVO 200, Beckman Coulter |
| | (inoculation into | Biomek FX, or equivalents |
| | production media) | |
| Colony pickers | for inoculating colonies | Scirobotics Pickolo, |
| | in liquid media | Molecular Devices QPix 420 |
| Platform shaker- | incubation with shaking | Kuhner Shaker ISF4-X, |
| incubators | of microtiter plate cultures | Infors-ht Multitron Pro |
| Liquid handlers | For transferring from | Hamilton Microlab STAR, |
| | culture plates to different | Labcyte Echo 550, Tecan |
| | culture plates (inoculation | EVO 200, Beckman Coulter |
| | into production media) | Biomek FX, or equivalents |
| Platform shaker- | incubation with shaking | Kuhner Shaker ISF4-X, |
| incubators | of microtiter plate cultures | Infors-ht Multitron Pro |
| liquid | Dispense liquid culture | Well mate (Thermo), |
| dispensers | media into microtiter | Benchcel2R (velocity 11), |
| | plates | plateloc (velocity 11) |
| microplate | apply barcoders to plates | Microplate labeler (a2+ cab - |
| labeler | | agilent), benchcell 6R |
| | | (velocity 11) |
| Liquid handlers | For transferring from | Hamilton Microlab STAR, |
| | culture plates to different | Labcyte Echo 550, Tecan |
| | culture plates | EVO 200, Beckman Coulter |
| | (inoculation into | Biomek FX, or equivalents |
| | production media) | |
| Platform | incubation with shaking | Kuhner Shaker ISF4-X, |
| shaker- | of microtiter plate | Infors-ht Multitron Pro |
| incubators | cultures | |
| liquid | Dispense liquid culture | well mate (Thermo), |
| dispensers | media into multiple | Benchcel2R (velocity 11), |
| | microtiter plates and seal | plateloc (velocity 11) |
| | plates | |
| microplate | Apply barcodes to plates | microplate labeler (a2+ cab - |
| labeler | | agilent), benchcell 6R |
| | | (velocity 11) |
| Liquid handlers | For processing culture | Hamilton Microlab STAR, |
| | broth for downstream | Labcyte Echo 550, Tecan |
| | analytical | EVO 200, Beckman Coulter |
| | | Biomek FX, or equivalents |
| UHPLC, HPLC | quantitative analysis of | Agilent 1290 Series UHPLC |
| | precursor and target | and 1200 Series HPLC with |
| | compounds | UV and RI detectors, or |
| | | equivalent; also any LC/MS |
| LC/MS | highly specific analysis | Agilent 6490 QQQ and 6550 |
| | of precursor and target | QTOF coupled to 1290 Series |
| | compounds as well as | UHPLC |
| | side and degradation | |
| | products | |
| Spectrophotometer | Quantification of | Tecan M1000, spectramax |
| | different compounds | M5, Genesys 10S |
| | using spectrophotometer | |
| | based assays | |
| Fermenters: | incubation with shaking | Sartorius, DASGIPs |
| | | (Eppendorf), BIO-FLOs |
| | | (Sartorius-stedim). Applikon |
| Platform shakers | | innova 4900, or any equivalent |

Fermenters: DASGIPs (Eppendorf), BIO-FLOs (Sartorius-stedim)

| | | |
|---|---|---|
| Liquid handlers | For transferring from | Hamilton Microlab STAR, |
| | culture plates to different | Labcyte Echo 550, Tecan |
| | culture plates (inoculation | EVO 200, Beckman Coulter |
| | into production media) | Biomek FX, or equivalents |
| UHPLC, HPLC | quantitative analysis of | Agilent 1290 Series UHPLC |
| | precursor and target | and 1200 Series HPLC with |
| | compounds | UV and RI detectors, or |
| | | equivalent; also any LC/MS |
| LC/MS | highly specific analysis | Agilent 6490 QQQ and 6550 |
| | of precursor and target | QTOF coupled to 1290 Series |
| | compounds as well as | UHPLC |
| | side and degradation | |
| | products | |
| Flow cytometer | Characterize strain | BD Accuri, Millipore Guava |
| | performance (measure | |
| | viability) | |
| Spectrophotometer | Characterize strain | Tecan M1000, Spectramax |
| | performance (measure | M5, or other equivalents |
| | biomass) | |

### Computer System Hardware

Provided herein is hardware that can be used with any of the computer systems described herein. A computer system may be used to execute program code stored in a non-transitory computer readable medium (e.g., memory) in accordance with any of the embodiments of the disclosure. A computer system can comprise an input/output subsystem, which may be used to interface with human users and/or other computer systems depending upon the application. The system may include, e.g., a keyboard, mouse, graphical user interface, touchscreen, or other interfaces for input, and, e.g., an LED or other flat screen display, or other interfaces for output, including application program interfaces (APIs). Other elements of embodiments of the disclosure, such as the components of the LIMS system, may be implemented with a computer system.

Program code may be stored in non-transitory media such as persistent storage in secondary memory or main memory or both. Main memory may include volatile memory such as random access memory (RAM) or non-volatile memory such as read only memory (ROM), as well as different levels of cache memory for faster access to instructions and data. Secondary memory may include persistent storage such as solid state drives, hard disk drives or optical disks. One or more processors can read program code from one or more non-transitory media and execute the code to enable the computer system to accomplish a method herein. Those skilled in the art will understand that the processor(s) may ingest source code, and interpret or compile the source code into machine code that is understandable at the hardware gate level of the processor(s). The processor(s) may include graphics processing units (GPUs) for handling computationally intensive tasks. Particularly in machine learning, one or more CPUs may offload the processing of large quantities of data to one or more GPUs.

In some embodiments, a processor(s) may communicate with external networks via one or more communications interfaces, such as a network interface card, WiFi transceiver, etc. A bus communicatively couples the I/O subsystem, the processor(s), peripheral devices, communications interfaces, memory, and persistent storage. Embodiments of the disclosure are not limited to this representative architecture.

As used herein, the term component in this context refers broadly to software, hardware, or firmware (or any combination thereof) component. Components are typically functional components that can generate useful data or other output using specified input(s). A component may or may not be self-contained. An application program (also called an “application”) may include one or more components, or a component can include one or more application programs.

The term “memory” can be any device or mechanism used for storing information. In accordance with some embodiments of the present disclosure, memory is intended to encompass any type of, but is not limited to: volatile memory, nonvolatile memory, and dynamic memory. For example, memory can be random access memory, memory storage devices, optical memory devices, magnetic media, floppy disks, magnetic tapes, hard drives, SIMMs, SDRAM, DIMMs, RDRAM, DDR RAM, SODIMMS, erasable programmable read-only memories (EPROMs), electrically erasable programmable read-only memories (EEPROMs), compact disks, DVDs, and/or the like. In accordance with some embodiments, memory may include one or more disk drives, flash drives, databases, local cache memories, processor cache memories, relational databases, flat databases, servers, cloud-based platforms, and/or the like. In addition, those of ordinary skill in the art will appreciate many additional devices and techniques for storing information can be used as memory.

In some embodiments, memory may be used to store instructions for running one or more applications or modules on a processor. For example, memory could be used in some embodiments to house all or some of the instructions needed to execute the functionality of one or more of the modules and/or applications disclosed in this application.

### Strategies for Stabilization of RNA

Provided herein are nucleic acids that may be used to for stable expression of RNAs in one or more host cells. As described herein, stable expression of RNAs may refer to mechanisms that increase one or more of (i) transcription levels of RNA, (ii) half-life of RNA in a cell, or (iii) efficiency of RNA translation.

In some embodiments, a DNA construct comprises a transgene that encodes one or more proteins. Accordingly, also provided are methods of expressing one or more proteins in a host cell, using the DNA constructs and/or stabilized RNAs disclosed herein. In some embodiments, use of the DNA constructs and/or stabilized RNAs may lead to (i) increased levels of protein expression in a host cell, (ii) increased half-life of the protein in a host cell, and/or (iii) increased accumulation of the protein in a host cell.

Stabilization of RNA can be achieved using a variety of approaches, such as any of those previously described. Exemplary methodologies comprise the use of elements in the construct which modulate transcriptional regulation and/or translational regulation. Illustrative methods for modulation of transcriptional regulation include codon optimization. Illustrative methods for modulation of translational regulation include modification of a promoter and/or terminator, codon optimization, modification of an intron sequence, insertion of exogenous intron sequences, insertion or modification of a 5′ and/or 3′ untranslated region (UTR), the use of a ubiquitin monomer, and any combination thereof.

In some embodiments, the DNA constructs of the disclosure can comprise one or more elements, and the elements may be provided in any order. For example, a DNA construct may comprise the following elements, in order from 5′ to 3′: promoter-signal sequence-transgene-KDEL-terminator. In some embodiments, a DNA construct may comprise an intron. In some embodiments, the intron is located within the transgene sequence, or 5′ or 3′ thereto. In some embodiments, the intron is located between the promoter and the signal sequence, between the signal sequence and the transgene, between the transgene and the KDEL, or between the KDEL and the terminator.

### Promoters

In some embodiments, stabilization of RNA in a host cell can be achieved via transcriptional regulation. For example, in some embodiments, transcriptional regulation of an RNA may be achieved by modulation of a promoter sequence in a DNA construct encoding the RNA. Modulation of a promoter can refer to modulation of an endogenous promoter, such as making one or more nucleotide substitutions relative to an endogenous promoter. In some embodiments, modulation of a promoter may refer to the addition of one or more exogenous promoters to a DNA construct.

In some embodiments, a DNA construct comprises a promoter that is capable of stably expressing an RNA in a cell. In some embodiments, a DNA construct comprises a promoter that is capable of increasing the level of RNA in a cell. In some embodiments, a DNA construct comprises a promoter that leads to increased half-life of an RNA in a cell. The promoters described herein may be derived in their entirety from a native gene or be composed of different elements derived from different promoters found in nature, or even comprise synthetic DNA segments.

In some embodiments, the promoter may be a plant promoter. A “plant promoter” is a promoter capable of initiating transcription in plant cells and can drive or facilitate transcription of a nucleotide sequence or fragment thereof of the instant invention. Such promoters need not be of plant origin. For example, promoters derived from plant viruses, such as the CaMV35S promoter or from Agrobacterium tumefaciens, such as the T-DNA promoters, can be plant promoters. A typical example of a plant promoter of plant origin is the maize ubiquitin-1 (ubi-1) promoter known to those of skill. Plant promoters can be from a monocot, dicot, Arabidopsis, rice, modified versions thereof, or combinations thereof.

Promoters can be selected based on the desired outcome, and may include constitutive, tissue-specific, inducible, or other promoters for expression in the host organism. In some embodiments, a promoter is a constitutive promoter. Promoters referred to herein as “constitutive promoters” actively promote transcription under most, but not necessarily all, environmental conditions and states of development or cell differentiation. Promoters from viral (Verdaguer et al., 1998; Schenk et al., 1999; Bohorova et al., 2001; Samac et al., 2004; Davies et al., 2014) and plant polyubiquitin (UBQ) genes (Lu et al., 2008; Mann et al., 2011) can be used to obtain enhanced constitutive transgene expression. Exemplary constitutive plant promoters comprise: Cauliflower Mosaic Virus 35S (35S), 1′ or 2′ promoter derived from T-DNA of Agrobacterium tumefaciens, maize ubiquitin-1, or modified versions of any of these.

In choosing a promoter to use in the methods of the disclosure, it may be desirable to use a tissue-specific or developmentally regulated promoter. In some cases, a promoter is a specific promoter. A specific promoter refers to a promoter that has a high preference for being active in a specific tissue or cell and/or at a specific time during development of a plant. By “high preference” is meant at least a 3-fold, preferably 5-fold, more preferably at least 10-fold still more preferably at least a 20-fold, 50-fold or 100-fold increase in transcription in the desired tissue over the transcription in any other tissue. Typical examples of temporal and/or tissue specific promoters of plant origin that can be used with the polynucleotides of the present invention, are: SH-EP from Vigna mungo and EP-C1 from Phaseolus vulgaris (Yamauchi et al. (1996) Plant Mol Biol. 30:321-9.); RCc2 and RCc3, promoters that direct root-specific gene transcription in rice (Xu et al. (1995) Plant Mol. Biol. 27:237) and TobRB27, a root-specific promoter from tobacco (Yamamoto et al. (1991) Plant Cell 3:371).

Promoters which are seed or embryo-specific and may be useful in disclosure include soybean Kunitz trypsin inhibitor (Kti3, Jofuku and Goldberg, Plant Cell 1:1079-1093 (1989)), patatin (potato tubers) (Rocha-Sosa, M., et al. (1989) EMBO J. 8:23-29), convicilin, vicilin, and legumin (pea cotyledons) (Rerie, W. G., et al. (1991) Mol. Gen. Genet. 259:149-157; Newbigin, E. J., et al. (1990) Planta 180:461-470; Higgins, T. J. V., et al. (1988) Plant. Mol. Biol. 11:683-695), zein (maize endosperm) (Schemthaner, J. P., et al. (1988) EMBO J. 7:1249-1255), phaseolin (bean cotyledon) (Segupta-Gopalan, C., et al. (1985) Proc. Natl. Acad. Sci. U.S.A. 82:3320-3324), phytohemagglutinin (bean cotyledon) (Voelker, T. et al. (1987) EMBO J. 6:3571-3577), β-conglycinin and glycinin (soybean cotyledon) (Chen, Z-L, et al. (1988) EMBO J. 7:297-302), glutelin (rice endosperm), hordein (barley endosperm) (Marris, C., et al. (1988) Plant Mol. Biol. 10:359-366), glutenin and gliadin (wheat endosperm) (Colot, V., et al. (1987) EMBO J. 6:3559-3564), and sporamin (sweet potato tuberous root) (Hattori, T., et al. (1990) Plant Mol. Biol. 14:595-604).

In some embodiments, a promoter can be a soybean promoter. In some cases, a promoter can be a soybean seed specific promoter. Exemplary suitable soybean promoters comprise: AtOle1, GmBg7S1, Gm2S-1, GmBBId-II, GmCons4, GmCons6, GmCons10, GmRoot1, GmRoot2, GmRoot3, GmRoot5, GmRoot6, GmRoot7, GmRoot8, GmSeed2, GmSeed3, GmSeed5, GmSeed6, GmSeed7, GmSeed8, GmSeed10, GmSeed11, GmSeed12, GmCEP1-L, GmGRD, GmFAB1, GmFAB2, GmFAB3, GmFAB5, GmFAB8, GmFAB9, GmFAB10, GmFAB11, GmFAB17, GmTHIC, GmOLEA, GmOLEB, GmWRKY13, GmWRKY17, GmWRKY21, GmWRKY27, GmWRKY43, GmWRKY54, GmWRKY67, GmWRKY79, GmWRKY80, GmWRKY82, GmWRKY85, GmWRKY162, PvDlec2, PvPhas, pBCON, LfKCS3, FAE1, BoACP, BnNap, BnaNapinC, SSPRO2745.1, SSPRO2743.1, modifications thereof, and any combination thereof.

In some embodiments, a promoter is selected from those provided in Table 4 or Table 11. In some embodiments, a promoter comprises a sequence having at least 70%, at least 80%, at least 85%, at least 90%, at least 95%, at least 97%, at least 98%, at least 99%, or 100% identity, including all ranges and subranges therebetween, with a sequence selected from Table 4 or Table 11. In some embodiments, a promoter is selected from the group that consists of: gmSeed2, gmSeed12, and pvPhas. In some embodiments, a DNA construct for stably expressing RNA in a host cell comprises a gmSeed2 promoter operably linked to a transgene. In some embodiments, a DNA construct for stably expressing RNA in a host cell comprises a gmSeed12 promoter operably linked to a transgene. In some embodiments, a DNA construct for stably expressing RNA in a host cell comprises a pvPhas promoter operably linked to a transgene.

In some embodiments, the promoter is an inducible promoter. Inducible promoters selectively express an operably linked DNA sequence in response to the presence of an endogenous or exogenous stimulus, for example by chemical compounds (chemical inducers) or in response to environmental, hormonal, chemical, and/or developmental signals. Inducible or regulated promoters include, for example, promoters regulated by light, heat, stress, flooding or drought, phytohormones, wounding, or chemicals such as ethanol, jasmonate, salicylic acid, or safeners.

Additional promoters for regulating the expression of the transgenes of the present disclosure in plants are stalk-specific promoters. Such stalk-specific promoters include the alfalfa S2A promoter (GenBank Accession No. EF030816; Abrahams et al., Plant Mol. Biol. 27:513-528 (1995)) and S2B promoter (GenBank Accession No. EF030817) and the like, herein incorporated by reference.

The location of promoters used in the DNA constructs designed herein may be selected to increase RNA expression, and/or downstream protein expression. For example, in some embodiments, the promoter may be located proximal to the transcriptional start site. In some embodiments, the promoter may be located distal to the transcriptional start site (i.e., it may be located thousands and more nucleotides adjacent, typically upstream, of the transcriptional start site.) In some embodiments, the promoter may be a minimal promoter. A “minimal promoter” may be, for example, a truncated or modified version of a wildtype promoter that includes substantially only those sequences required to properly initiate transcription.

In some cases, a promoter can be paired with a transcription terminator to achieve improved RNA stability and/or transgene expression in plants. Transcriptional termination is the process by which RNA synthesis by RNA polymerase is substantially stopped, and both the processed messenger RNA and the enzyme are released from the DNA template. In some cases, improper termination of an RNA transcript can affect the stability of the RNA, and hence can affect protein expression. Variability of transgene expression is sometimes attributed to variability of termination efficiency.

### Terminator

A “transcriptional terminator” or “terminator” is a nucleic acid sequence that can halt transcription. It comprises a DNA sequence involved in specific termination of RNA transcription by RNA polymerase. Transcriptional terminator sequences can prevent transcriptional activation of downstream nucleic acid sequences by upstream promoters. A transcriptional terminator may be required in vivo to achieve the desired level of expression or to avoid transcription of a particular sequence. A transcription terminator is considered operably linked to a nucleotide sequence if it can reduce or eliminate transcription of the sequence to which it is linked. In some embodiments, the terminator is a forward terminator. Normally, a forward terminator interrupts transcription when placed upstream of a nucleic acid sequence to be transcribed. In some embodiments, the terminator is a bi-directional terminator. A bi-directional terminator may stop transcription for both the forward and reverse strands and may have the capability of terminating transcription in both 5′ to 3′, and 3′ to 5′ orientations. A single sequence element that acts as a bidirectional terminator can terminate transcription initiated from two convergent promoters.

In some embodiments, the terminator is a reverse transcription terminator, which typically terminates transcription upon reverse strand swallowing.

Terminator sequences can contain polyadenylation (poly(A)) signals, which control the steps involved in 3′ end formation: recognition, endonucleolytic cleavage, and polyadenylation of primary RNA (pre-mRNA). These steps can impact gene expression by influencing mRNA termination, stability, localization, export to cytoplasm, and/or translation efficiency. In some embodiments, a terminator is selected from those provided in Table 4 or Table 11. In some embodiments, a terminator comprises a sequence having from 70%, 80%, 85%, 90%, 95%, 97%, 98%, 99%, or 100% identity, including all ranges and subranges therebetween, with a sequence selected from Table 4 or Table 11.

In eukaryotic systems, such as in plants, a terminator may contain special DNA sequences that allow site-specific cleavage of new transcripts to expose polyadenylation sites. This signals a specialized endogenous polymerase, adding a stretch of about 200 A residues (polyA) to the 3′ end of the transcript. RNA molecules modified with this poly A tail are believed to be more stable and more efficiently translated. Thus, in some embodiments, the terminator may include a signal for RNA cleavage. In some embodiments, the terminator signal promotes polyadenylation of the message. Terminators and/or elements of the polyadenylation site can serve to enhance the output nucleic acid levels and/or to minimize readthrough between nucleic acids.

In some embodiments, a DNA construct comprises a terminator that promotes stable expression of an RNA in a cell. In some embodiments, a DNA construct comprises a terminator that is capable of increasing the level of RNA in a cell. In some embodiments, a DNA construct comprises a terminator that leads to increased half-life of an RNA in a cell. In some embodiments, a DNA construct comprises a combination of a promoter and a terminator that is capable of increasing the level of RNA in a cell. In some embodiments, a DNA construct comprises a combination of a promoter and a terminator that leads to increased half-life of an RNA in a cell.

In some embodiments, terminators for use in accordance with the present disclosure include any terminators described herein or known to those of skill in the art. Examples of terminators include, but are not limited to, termination sequences of genes such as, for example, bovine growth hormone terminator, and, for example, NOS, ARC, EU, Rb7, HSP, ATHSP, AtUbi10, Stubi3, TM6, Octopine Synthase (OCS), SV40 terminator, spy, yejM, secG-leuU, thrLABC, rmB T1, hisLGDCBHAFI, metZWV, rmC, xapR, aspA, EU:Rb7, AtHSP:AtUbi10, EU:StUbi3, EU:TM6. In some embodiments, the terminator comprises a virus termination sequences such as an arcA terminator. In some embodiments, the terminator may be a sequence that cannot be transcribed or translated, such as that resulting from sequence truncation. In some embodiments, a terminator is a dual terminator and is selected from the group consisting of: EU:Rb7, AtHSP:AtUbi10, EU:StUbi3, and EU:TM6. In some embodiments, a terminator is selected from the group consisting of NOS, ARC, EU, Rb7, HSP, AtHSP, AtUbi10, Stubi3, and TM6.

Any heterologous polynucleotide of interest can be operably linked to a terminator sequence provided in the disclosure. Examples of polynucleotides of interest that can be operably linked to the terminator sequences described herein include, but are not limited to, polynucleotides comprising regulatory elements such as introns, enhancers, promoters, translation leader sequences, protein-coding regions from disease and insect resistance genes, genes conferring nutritional value, genes conferring yield and heterosis increase, genes that confer male and/or female sterility, antifungal, antibacterial or antiviral genes, selectable marker genes, herbicide resistance genes and the like.

In embodiments, RNA stabilization can be achieved via combination of a terminator and promoter provided herein. In some embodiments, a synergistic effect on RNA stabilization is observed when a terminator and a promoter are present in a DNA construct of the disclosure. In some embodiments, RNA stabilization is increased by at least about 1-fold, about 2-fold, about 3-fold, about 5-fold, about 10-fold, about 20-fold, about 50-fold, about 100-fold, about 300-fold, about 500-fold, or about 1000-fold, including all ranges and subranges therebetween, when a promoter and terminator are present in a DNA construct as compared to an otherwise comparable construct lacking at least one of the promoter or terminator.

### Intron

In some embodiments, RNA stabilization can be achieved or improved via the addition or removal of endogenous or exogenous intronic sequences in a DNA construct. Introns are non-coding sections of an RNA transcript that can be removed by RNA splicing during maturation of the final RNA product.

Intron sequences may be incorporated at any location within a DNA construct, including but not limited to a 5′ end, 3′ end, within or adjacent to a transgene sequence, and any combination thereof. In some cases, an intron sequence is added within a transgene sequence. In some cases, an intron is located within about 0-5, 1-10, 5-25, or 10-30 bases from a start of a transgene sequence. In some cases, an intron is located up to about 0, 5, 10, 15, 20, 30, 40, 50, 70, 90, or 100 bases from a start of a transgene sequence. In some embodiments, an intron is placed adjacent to a 5′UTR. In some embodiments, an intron is placed within a coding sequence of a transgene. In some embodiments, an intron is placed after a promoter sequence. In some embodiments, an intron is placed between a promoter sequence and a coding sequence. In some cases, an endogenous or native intron is replaced with an exogenous intron. Replacement may be full replacement or partial replacement. In cases comprising partial intron replacement, a portion of an endogenous intron remains and can be adjacent to an exogenous intron sequence.

In some embodiments, an intron sequence used in a DNA construct is isolated or derived from a eukaryote. For example, the intron may be isolated or derived from an intronic sequence of a eukaryote selected from animals, plants, and fungi. In some cases, an intron sequence may be isolated or derived from a plant. The plant intronic sequence may be from the same plant species as a host cell, a different plant species as compared to a host cell, or a hybrid species. In some embodiments, an intron sequence isolated or derived from glycine max, Arabidopsis thaliana, or both. In some embodiments, an intron sequence is isolated or derived from a soybean (Glycine max). In some embodiments, an intron sequence is isolated or derived from elongation factor TA.

The DNA constructs described herein may comprise any number of introns. For example, a DNA construct may comprise 1, 2, 3, 4, 5, 6, 7, 8, 9, 10, or more introns. In some embodiments, a DNA construct comprises a transgene, wherein the transgene comprises 1, 2, 3, 4, 5, 6, 7, 8, 9, 10, or more introns. In some embodiments, a transgene may comprise a reduced number of introns relative to the wildtype gene. For example, in some embodiments, 1, 2, 3, 4, 5, 6, 7, 8, 9, 10, or more introns may be removed from a wildtype gene to produce a transgene as described herein. In some embodiments, a transgene sequence comprises about 1 to about 3 intronic sequences. In some cases, a transgene comprises one intron, two introns, or three introns.

In some embodiments, an intron comprises a sequence having at least 70%, at least 80%, at least 85%, at least 90%, at least 95%, at least 97%, at least 98%, at least 99%, or 100% identity, including all ranges and subranges therebetween, with a sequence selected from Table 11 or Table 14.

### Untranslated Regions (UTRs)

In some embodiments, RNA stabilization can be achieved or improved via addition or removal of 5′ or 3′ untranslated regions (5′UTR or 3′UTR) to a DNA construct. UTRs are known to control gene expression and protein function via a wide range of mechanisms. Exemplary mechanisms include: (A) Alternative polyadenylation: either more than one polyA site is utilized to produce mRNA variants that differ in 3′UTR length or, if the polyA is located upstream of the stop codon, truncated transcripts are produced; (B) Riboswitching: 3′ or 5′UTRs form folding structures that sense a metabolite and modulate transcript stability; (C) Adenosine methylation (m6A): The presence of m6A in 5′UTR promotes CAP-independent translation; (D) Short-peptide translation: A short open reading frame in 5′UTR (uORF) can either repress the translation of the main ORF or induce mRNA decay via the NMD pathway; (E) Nonsense-mediated decay (NMD): A pre-mature termination codon (PTC) upstream of the regular termination codon (TC) recruits NMD factors that mediate mRNA decay; (F) Alternative splicing: Retention of intronic elements in 5′UTR can either promote or repress translation, while retention of intronic elements in 3′UTR can modulate miRNA-mediated cleavage. In some embodiments, a 5′UTR and/or 3′ UTR is modulated to regulate (increase or decrease) the transport of an mRNA out of a plant nucleus. Any one of the aforementioned mechanisms can be employed in disclosed strategies to stabilize RNA in a plant.

5′ untranslated regions (UTRs) play an important role in optimizing gene expression. 5′UTR are short sequences (˜65 bp) upstream of the start codon (AUG) that can affect translation initiation by its secondary structure and the existence of the AUG. The κ′ UTR can have a positive or negative effect on translation since they are the target for the binding of microRNAs. Additionally, 5′ UTRs can have a role in mRNA stabilization. In some embodiments, a DNA construct described herein comprise a 5′ UTR. In some embodiments, a DNA construct described herein comprises a 3′ UTR. In some embodiments, a DNA construct described herein comprises a 5′ UTR and a 3′UTR.

In some embodiments, a UTR (e.g., a 5′ UTR or a 3′ UTR) comprises a sequence that is isolated or derived from a plant. For example, in some embodiments, a UTR can comprise a sequence that is isolated or derived from a soybean plant. In some embodiments, a UTR comprises a sequence that is isolated or derived from a mammal, such as any of the mammals described herein. In some embodiments, a UTR comprises a sequence that is isolated or derived from a gene encoding a milk protein, such as β-Lactoglobulin. In some embodiments, a UTR comprises a sequence that is isolated or derived from a gene encoding an egg protein, such as ovalbumin. In some embodiments, a DNA construct described herein comprise a 5′UTR selected from: Arc5′UTR, glnB1UTR, native UTRs of ovalbumin, native UTR of β lactoglobulin, and combinations thereof. In some embodiments, a UTR is selected from those provided in Table 4, or Table 11. In some embodiments, a UTR comprises a sequence having at least 70%, at least 80%, at least 85%, at least 90%, at least 95%, at least 97%, at least 98%, at least 99%, or 1000 identity, including all ranges and subranges therebetween, with a sequence selected from Table 4 or Table 11.

TABLE 4
Exemplary promoters, 5′ UTRs, signal peptides and terminators. Disclosed
are also variants thereof, homologues thereof, and modified versions thereof.

| | | | | Illustrative |
|---|---|---|---|---|
| | | | | Accession No. |
| Type | Name | Description | Native Species | (Glyma, GenBank) |
| Promoter | PvPhas | Phaseolin-1 (aka β- | Common bean | J01263.1 |
| | | phaseolin) | (Phaseolus vulgaris) | |
| | BnNap | Napin-1 | Rapeseed (Brassica | J02798.1 |
| | | | napus) | |
| | AtOle1 | Oleosin-1 (Ole1) | Arabidopsis | X62353.1, |
| | | | (Arabidopsis | AT4G25140 |
| | | | thaliana) | |
| | GmSeed2 | Gy1 (Glycinin 1) | Soybean (Glycine | Glyma.03G163500 |
| | | | max) | |
| | GmSeed3 | cysteine protease | Soybean (Glycine | Glyma.08G116300 |
| | | | max) | |
| | GmSeed5 | Gy5 (Glycinin 5) | Soybean (Glycine | Glyma.13G123500 |
| | | | max) | |
| | GmSeed6 | Gy4 (Glycinin 4) | Soybean (Glycine | Glyma.10G037100 |
| | | | max) | |
| | GmSeed7 | Kunitz trypsin protease | Soybean (Glycine | Glyma.01G095000 |
| | | inhibitor | max) | |
| | GmSeed8 | Kunitz trypsin protease | Soybean (Glycine | Glyma.08G341500 |
| | | inhibitor | max) | |
| | GmSeed10 | Legume Lectin Domain | Soybean (Glycine | Glyma.02G012600 |
| | | | max) | |
| | GmSeed11 | β-conglycinin a subunit | Soybean (Glycine | Glyma.20G148400 |
| | | | max) | |
| | GmSeed12 | β-conglycinin a′ subunit | Soybean (Glycine | Glyma.10G246300 |
| | | | max) | |
| | pBCON | β-conglycinin β subunit | Soybean (Glycine | Glyma.20G148200 |
| | | | max) | |
| | GmCEP1-L | KDEL-tailed cysteine | Soybean (Glycine | Glyma06g42780 |
| | | endopeptidase CEP1-like | max) | |
| | GmTHIC | phosphomethylpyrimidine | Soybean (Glycine | Glyma11g26470 |
| | | synthase | max) | |
| | GmBg7S1 | Basic 7S globulin precursor | Soybean (Glycine | Glyma03g39940 |
| | | | max) | |
| | GmGRD | glucose and ribitol | Soybean (Glycine | Glyma07g38790 |
| | | dehydrogenase-like | max) | |
| | GmOLEA | Oleosin isoform A | Soybean (Glycine | Glyma.19g063400 |
| | | | max) | |
| | GmOLEB | Oleosin isoform B | Soybean (Glycine | Glyma.16g071800 |
| | | | max) | |
| | Gm2S-1 | 2S albumin | Soybean (Glycine | Glyma13g36400 |
| | | | max) | |
| | GmBBId-II | Bowman-Birk protease | Soybean (Glycine | Glyma16g33400 |
| | | inhibitor | max) | |
| 5′UTR | Arc5′UTR | arc5-1 gene | Phaseolus vulgaris | J01263.1 |
| | glnB1UTR | 65 bp of native glutamine | Soybean (Glycine | AF301590.1 |
| | | synthase | max) | |
| Signal peptide | GmSCB1 | Seed coat BURP domain | Soybean (Glycine | Glyma07g28940.1 |
| | | protein | max) | |
| | StPat21 | Patatin | Tomato (Solanum | CAA27588 |
| | | | lycopersicum) | |
| | 2Sss | 2S albumin | Soybean (Glycine | Glyma13g36400 |
| | | | max) | |
| | Sig2 | Glycinin G1 N-terminal | Soybean (Glycine | Glyma.03G163500 |
| | | peptide | max) | |
| | Sig12 | Beta-conglycinin alpha | Soybean (Glycine | Glyma.10G246300 |
| | | prime subunit N-terminal | max) | |
| | | peptide | | |
| | Sig8 | Kunitz trypsin inhibitor N- | Soybean (Glycine | Glyma.08G341500 |
| | | terminal peptide | max) | |
| | Sig10 | Lectin N-terminal peptide | Soybean (Glycine | Glyma.02G012600 |
| | | from Glycine max | max) | |
| | Sig11 | Beta-conglycinin alpha | Soybean (Glycine | Glyma.20G148400 |
| | | subunit N-terminal peptide | max) | |
| | Coixss | Alpha-coixin N-terminal | Coix lacryma-job | |
| | | peptide from Coix lacryma- | | |
| | | job | | |
| | KDEL | C-terminal amino acids of | Phaseolus vulgaris | |
| | | sulfhydryl endopeptidase | | |
| Terminator | NOS | Nopaline synthase gene | Agrobacterium | |
| | | termination sequence | tumefaciens | |
| | ARC | arc5-1 gene termination | Phaseolus vulgaris | J01263.1 |
| | | sequence | | |
| | EU | Extensin termination | Nicotiana tabacum | |
| | | sequence | | |
| | Rb7 | Rb7 matrix attachment | Nicotiana tabacum | |
| | | region termination | | |
| | | sequence | | |
| | HSP or AtHSP | Heat shock termination | Arabidopsis thaliana | |
| | | sequence | | |
| | AtUbi10 | Ubiquitin 10 termination | Arabidopsis thaliana | |
| | | sequence | | |
| | Stubi3 | Ubiquitin 3 termination | Solanum tuberosum | |
| | TM6 | M6 matrix attachment | Nicotiana tabacum | |
| | | region termination | | |
| | | sequence | | |
| Dual terminators | EU:Rb7 | Extensin termination | Nicotiana tabacum | |
| | | sequence:Rb7 matrix | | |
| | | attachment region | | |
| | | termination sequence | | |
| | AtHSP:AtUbi10 | Heat shock termination | Arabidopsis thaliana | |
| | | sequence:Ubiquitin 10 | | |
| | | termination sequence | | |
| | EU:StUbi3 | Rb7 matrix attachment | Nicotiana tabacum, | |
| | | region termination | Solanum tuberosum | |
| | | sequence:Ubiquitin 3 | | |
| | | termination | | |
| | EU:TM6 | Rb7 matrix attachment | Nicotiana tabacum | |
| | | region termination | | |
| | | sequence:M6 matrix | | |
| | | attachment region | | |
| | | termination sequence | | |

### Ubiquitin Monomer

In some embodiments, RNA can be stabilized via use of a ubiquitin monomer. Ubiquitin is a small protein that can be covalently linked to lysine residues of proteins targeted for intracellular degradation by proteasomes. Expression of recombinant/transgenic proteins fused with a ubiquitin monomer can be an advantageous strategy to enhance protein accumulation. The ubiquitin monomer may act as a chaperone for the incorporation of the ribosomal protein into the ribosome. During translation, ubiquitin can be accurately cleaved from the protein by endogenous ubiquitin-specific proteases, leaving the protein of interest free of unnecessary sequences.

In some embodiments, ubiquitin monomers from plants can be utilized in DNA constructs in order to enhance protein expression. The ubiquitin monomer is cleaved either immediately after or during translation improving translational regulation.

In some embodiments, a ubiquitin monomer is from a plant, a mammal, or a fungus. Any of the disclosed plants provided herein can be the source of a ubiquitin monomer, including but not limited to soybean, potato, wheat, corn, and the like. In some cases, the ubiquitin monomer is isolated or derived from a potato.

A ubiquitin monomer sequence can be located at any position of a transgene. In some cases, a monomer is at a 5′ end, 3′ end, in or adjacent to a coding sequence or promoter sequence, and combinations thereof. In some embodiments, a monomer is adjacent to a promoter sequence. In some embodiments, a monomer is located 3′ to a promoter sequence. In some embodiments, a promoter is located 5′ to any sequence provided herein. In some embodiments, a monomer is located within about 0-5, 1-10, 5-25, or 10-30 bases 5′ or 3′ of any sequence provided herein. In some embodiments, a monomer is located between a promoter and a signal peptide.

In some embodiments, a ubiquitin monomer comprises a sequence having at least 70%, at least 80%, at least 85%, at least 90%, at least 95%, at least 96%, at least 97%, at least 98%, at least 99%, or 100% identity, including all ranges and subranges therebetween, with a sequence selected from Table 11.

### Codon Optimization

In some embodiments, the present disclosure teaches that RNA stabilization can also be achieved through codon optimization of the DNA sequence encoding the RNA. In some embodiments, codon optimized variants of genes produce pluralities of nucleic acid sequences that can be evaluated for expression potential and stability via the methods of the present disclosure.

The genetic code consists of three-nucleotide units called codons. There are 64 possible codons, each specifying one of twenty amino acids or an end to translation (“STOP codons”). Therefore, at least some codons are redundant. In the coding system used by the vast majority of organisms, two amino acids are each encoded by a single codon, whereas all other amino acids are separately encoded by two, three, four, or six codons, with three STOP codons. For amino acids represented by two, three, or four codons, the codons typically differ from each other at the third nucleotide position. For amino acids represented by two codons, the third position is either a purine (A, G) or pyrimidine (C, T) in both cases. For the three amino acids that are represented by six codons (Arg, Leu, and Ser), each has one block of four codons that follows this pattern by differing in the third position, plus one additional set of two codons. Arg and Leu are each represented by a two-codon block different from each other by a change in the first and second nucleotide positions. The two-codon representation of serine (Ser) is different from that of the Arg two-codon block only in the third nucleotide position.

For a particular amino acid, a given organism does not use the possible codons equally. Organisms each have a bias in codon usage. The pattern of bias in codon usage is distinct for an organism and its close relatives throughout the genome. For example, in Streptomyces spp., frequent codons generally include G or C in the third nucleotide position. Rare codons generally include A or T in the third position. In other organisms, A or T is preferred in the third position. Within a particular species, there can be distinct categories of genes with their own codon bias. In E. coli, for example, there are roughly three classes of genes, each with a distinctive codon usage signature. One class is rich in important proteins that are abundantly expressed; the second class includes proteins that are expressed at relatively low levels; and the third class includes proteins likely to have been recently acquired from other species.

In most synthetic gene design strategies, the process attempts to match the codon composition of a synthetic gene to the codon compositions of genes of a host in which the synthetic gene will be expressed. See, e.g., U.S. Patent Publication No. US2007/0292918. Such strategies may in some situations lead to increased expression of the synthetic gene in the host. For example, codon optimization in yeast may significantly improve the translation of heterologous gene transcripts due to minimizing the effects of, e.g., limiting aminoacyl-tRNAs and transcription termination at AT-rich sequences. See, e.g., Daly and Hearn (2004) J. Mol. Recognition 18:119-38.

Codon optimization comprises a variety of approaches that involve synonymous substitutions to increase protein expression. One strategy that is preferred by some is to maximize the use of frequent codons in the expression host species during the design of heterologous genes. A second strategy preferred by others is to place maximum value on the context of particular codons, and therefore to maximize the use of codon pairs that occur frequently in the expression host. For example, in some embodiments, codon optimization pursues a codon harmonization approach, which seeks to maintain regions of slow translation that are thought to be important for protein folding.

A third strategy is to make the codon usage of the new coding sequence in the new species resemble the codon usage of the reference coding sequence in the species of origin. This third strategy places high value on the recognition of possible requirements for rare codons to ensure proper secondary structure of transcript RNA molecules. A further strategy is to make the codon composition of the heterologous gene resemble the overall codon composition of expressed genes of the new host. Sequence changes resulting in synonymous codons can also be used to alter numerous features of mRNA coding sequences that can inhibit expression, including putative splice donor and acceptor sites. Additionally, simply using the same frequently-occurring codon repeatedly in a heterologous sequence is expected to eventually have the same effect as selecting a rare codon, e.g., overuse of the corresponding tRNA will limit the availability of the tRNA. Thus, in some embodiments codon optimization should also seek to balance these strategies and their underlying concerns in order to produce best results.

Persons having skill in the art will be familiar with how to deploy codon-optimization techniques. Codon usage tables for almost all characterized organisms can be found online, including, the Kazusa database (world wide web at.kazusa.or.jp/codon/) and hive database (hive.biochemistry.gwu.edu/review/codon). In addition, a non-limiting list of software tools capable of generating codon optimized sequence variants is provided in Table 5, below.

TABLE 5
Codon Optimization Tools

| Codon Optimization Tool | Relevant Citation |
|---|---|
| DNAWorks | Hoover D. M., Lubkowski J. (2002). |
| | DNAWorks: an automated method for |
| | designing oligonucleotides for PCR-based |
| | gene synthesis. Nucleic Acids Res. 30, e43. |
| | 10.1093/nar/30.10.e43 |
| Jcat | Grote A., Hiller K., Scheer M., Munch R., |
| | Nortemann B., Hempel D. C., et al. (2005). |
| | JCat: a novel tool to adapt codon usage of a |
| | target gene to its potential expression host. |
| | Nucleic Acids Res. 33, W526-W531 |
| | 10.1093/nar/gki376 |
| Synthetic gene designer | Wu G., Bashir-Bello N., Freeland S. (2005). |
| | “The synthetic gene designer: a flexible web |
| | platform to explore sequence space of |
| | synthetic genes for heterologous |
| | expression,” in 2005 IEEE Computational |
| | Systems Bioinformatics Conference, |
| | Workshops and Poster Abstracts, 2005 Aug. |
| | 8-11. (California: Stanford University;), |
| | 258-259 |
| GeneDesign | Richardson S. M., Wheelan S. J., Yarrington |
| | R. M., Boeke J. D. (2006). GeneDesign: |
| | rapid, automated design of multikilobase |
| | synthetic genes. Genome Res. 16, 550-556 |
| | 10.1101/gr.4431306 |
| Gene Designer 2.0 | Villalobos A., Ness J. E., Gustafsson C., |
| | Minshull J., Govindarajan S. (2006). Gene |
| | designer: a synthetic biology tool for |
| | constructing artificial DNA segments. BMC |
| | Bioinformatics 7, 285. 10.1186/1471-2105- |
| | 7-285 |
| OPTIMIZER | Puigbò P., Guzmán E., Romeu A., Garcia- |
| | Vallvé S. (2007). Optimizer: a web server |
| | for optimizing the codon usage of DNA |
| | sequences. Nucleic Acids Res. 35, W126- |
| | W131 10.1093/nar/gkm219 |
| Visual gene developer | Jung S.-K., McDonald K. (2011). Visual |
| | gene developer: a fully programmable |
| | bioinformatics software for synthetic gene |
| | optimization. BMC Bioinformatics 12, 340. |
| | 10.1186/1471-2105-12-340 |
| Eugene | Gaspar P., Oliveira J. L., Frommlet J., |
| | Santos M. A. S., Moura G. (2012). EuGene: |
| | maximizing synthetic gene design for |
| | heterologous expression. Bioinformatics 28, |
| | 2683-2684 10.1093/bioinformatics/bts465 |
| COOL | Chin J. X., Chung B. K.-S., Lee D.-Y. |
| | (2014). Codon optimization on-line |
| | (COOL): a web-based multi-objective |
| | optimization platform for synthetic gene |
| | design. Bioinformatics 30, 2210-2212 |
| | 10.1093/bioinformatics/btu192 |
| D-Tailor | Guimaraes J. C., Rocha M., Arkin A. P., |
| | Cambray G. (2014). D-Tailor: automated |
| | analysis and design of DNA sequences. |
| | Bioinformatics 30, 1087-1094 |
| | 10.1093/bioinformatics/btt742 |

### Nucleic Acid Secondary Structure

Codon changes also have the potential to affect structure of nucleic acids in vivo. Specifically changes in primary sequence can affect folding, and therefore RNA stability. Therefore, in some embodiments, the present disclosure teaches methods for selecting primary transcript sequences based on their predicted secondary structures. Codon usage bias can be analyzed and optimized using various techniques. In some embodiments, a plurality of different codon optimized transgenes can be generated and evaluated for their RNA structure in silico, using publicly available programs such as RNAfold. Different parameters, such as thermodynamic parameters, can be analyzed including but not limited to minimum free energy structures (MFE), base pair probabilities, and energy mountain plot. In addition, locations of 5′ regions and/or start codons within different structures, such as MFE, can be determined to further analyze RNA structure within those regions to optimize as necessary. In some embodiments, using information gathered from thermodynamic parameter analysis, codon optimized sequences can be selected that yield RNA sequences that comprise a stable structure. Exemplary stable structures can comprise secondary structures, such as loops, bulges, base pair mismatches, hairpin loops, internal loops, helices, multibranch loops, terminal mismatches, dangling ends, and combinations thereof.

In some embodiments, a stable RNA structure comprises a loop. A loop of the disclosure can be of any length. In some cases, a loop of the disclosure that confers increased stability comprises at least about 4, 5, 6, 7, 8, 9, 10, 11, 12, 13, 14, 15, 16, 17, 18, 19, 20, 21, 22, 23, 24, 25, 26, 27, 29, 29, 30, or more base pairs. In some embodiments, a stable structure comprises a loop with a length of about 4 to about 8 base pairs. Additional exemplary stable structures can contain tetraloops, such as UUCG (SEQ ID NO: 615). In some embodiments, a stable structure comprises two or more hairpin loops, wherein the hairpin loops are each less than 8 base pairs.

In some embodiments, codon usage bias can be utilized to reduce or prevent unstable structures. An unstable structure can comprise a loop of less than about 4 base pairs or over 8 base pairs. In some embodiments, an unstable structure may lack a secondary structure. In some embodiments, an unstable structure comprises at least one of a pseudo-knot (e.g. a loop with no secondary structure), a loop of less than 4 base pairs or over 8 base pairs, or a large hairpin loop (e.g. over than about 8 base pairs).

In some embodiments, a sequence can be codon optimized for expression in a host cell, such as a plant cell. Other host cells are also contemplated. For example, a sequence can be codon optimized for expression in any of the plants of the disclosure. In some embodiments, a sequence is codon optimized for expression in a soybean plant (Glycine max).

### KDEL (Lys-Asp-Glu-Leu, SEQ ID NO: 616) and Related Sequences

Additionally, stabilization of RNA can be achieved through the use of a KDEL sequence. KDEL is a target peptide sequence in mammals and plants located on the C-terminal end of an amino acid structure of a protein. The KDEL sequence reduces or eliminates a protein from being secreted from the endoplasmic reticulum (ER) and can facilitate its return if it is exported. A protein with a functional KDEL motif will be retrieved from the Golgi apparatus by retrograde transport to the ER lumen. It also targets proteins from other locations (such as the cytoplasm) to the ER. Proteins can leave the ER after this sequence has been cleaved off. The instant inventors have surprisingly discovered that the presence of a KDEL sequence in an RNA may increase the stability thereof. Accordingly, provided herein are stably expressed RNA sequences comprising a sequence encoding a KDEL sequence. Also provided herein are DNAs comprising a KDEL sequence, that are capable of stably expressing an RNA in a host cell.

Homologues of KDEL are also contemplated in the present disclosure. A homologue may be a similar sequence employed in other organisms. For example, the sequence HDEL (His-Asp-Glu-Leu) performs the same function in yeasts as KDEL. In some embodiments, a DNA sequence described herein may comprise a sequence encoding any one of a KDEL, HDEL, and the like. In some embodiments, a DNA sequence described herein may comprise a sequence selected from the group consisting of: KDEL (SEQ ID NO: 616), HDEF (SEQ ID NO: 632), HDEL (SEQ ID NO: 633), RDEF (SEQ ID NO: 634), RDEL (SEQ ID NO: 635), WDEL (SEQ ID NO: 636), YDEL (SEQ ID NO: 637), HEEF (SEQ ID NO: 638), HEEL (SEQ ID NO: 639), KEEL (SEQ ID NO: 640), REEL (SEQ ID NO: 641), KAEL (SEQ ID NO: 642), KCEL (SEQ ID NO: 643), KFEL (SEQ ID NO: 644), KGEL (SEQ ID NO: 645), KHEL (SEQ ID NO: 646), KLEL (SEQ ID NO: 647), KNEL (SEQ ID NO: 648), KQEL (SEQ ID NO: 649), KREL (SEQ ID NO: 650), KSEL (SEQ ID NO: 651), KVEL (SEQ ID NO: 652), KWEL (SEQ ID NO: 653), KYEL (SEQ ID NO: 654), KEDL (SEQ ID NO: 655), KIEL (SEQ ID NO: 656), DKEL (SEQ ID NO: 657), FDEL (SEQ ID NO: 658), KDEF (SEQ ID NO: 659), KKEL (SEQ ID NO: 660), HADL (SEQ ID NO: 661), HAEL (SEQ ID NO: 662), HIEL (SEQ ID NO: 663), HNEL (SEQ ID NO: 664), HTEL (SEQ ID NO: 665), KTEL (SEQ ID NO: 666), HVEL (SEQ ID NO: 667), NDEL (SEQ ID NO: 668), QDEL (SEQ ID NO: 669), REDL (SEQ ID NO: 670), RNEL (SEQ ID NO: 671), RTDL (SEQ ID NO: 672), RTEL (SEQ ID NO: 673), SDEL (SEQ ID NO: 674), TDEL (SEQ ID NO: 675), SKEL (SEQ ID NO: 676), STEL (SEQ ID NO: 677), and EDEL (SEQ ID NO: 678).

In some embodiments, a sequence of the disclosure can be modified to add a KDEL sequence or a homologue thereof. In some embodiments, a sequence is modified to remove a KDEL sequence or homologue thereof. In some embodiments, a sequence can be modulated to potentiation, reduce, or otherwise strengthen or dampen an existing KDEL sequence or homologue thereof.

### Transgenes, Including Chordate Proteins

The DNA constructs described herein may comprise one or more transgenes. The transgenes may encode one or more of a protein or RNA of interest. In some embodiments, the transgene encodes a protein. In some embodiments, the transgene encodes a chordate protein. The chordate proteins provided herein may comprise proteins of a variety of chordates. Chordates are divided into three subphyla: Vertebrata (fish, amphibians, reptiles, birds, and mammals); Tunicata or Urochordata (sea squirts, salps); and Cephalochordata (which includes lancelets). Proteins from any of the aforementioned chordates can be utilized in compositions and methods of the disclosure.

In some embodiments, the chordate is a mammal. Accordingly, in some embodiments, the transgene is a mammalian protein. In some embodiments, the mammalian protein can comprise one or more milk proteins. As used herein the term “milk protein” refers to any protein, or fragment or variant thereof, that is typically found in one or more mammalian milks. Caseins and whey proteins are the major proteins of milk. Casein constitutes approximately 80% (29.5 g/L) of the total protein in bovine milk, and whey protein accounts for about 20% (6.3 g/L). Casein is chiefly phosphate-conjugated and mainly consists of calcium phosphate-micelle complexes. It is a heterogeneous family of 4 major components including alpha- (αs1- and αs2-casein), beta-, gamma-, para-κ-casein, and kappa-casein.

Illustrative milk proteins that may be used in a transgene of the disclosure include members of the casein family of proteins, such as α-S1 casein, α-S2 casein, β-casein, and κ-casein. The caseins are phosphoproteins and make up approximately 80% of the protein content in bovine milk and about 20-45% of the protein in human milk. Caseins form a multi-molecular, granular structure called a casein micelle in which some enzymes, water, and salts, such as calcium and phosphorous, are present. The micellar structure of casein in milk is significant in terms of a mode of digestion of milk in the stomach and intestine and a basis for separating some proteins and other components from cow milk. In practice, casein proteins in bovine milk can be separated from whey proteins by acid precipitation of caseins, by breaking the micellar structure by partial hydrolysis of the protein molecules with proteolytic enzymes, or microfiltration to separate the smaller soluble whey proteins from the larger casein micelle. Caseins are relatively hydrophobic, making them poorly soluble in water.

In some embodiments, the casein proteins described herein (e.g., α-S1 casein, α-S2 casein, β-casein, and/or κ-casein) are isolated or derived from cow (Bos taurus), goat (Capra hircus), sheep (Ovis aries), water buffalo (Bubalus bubalis), dromedary camel (Camelus dromedaries), bactrian camel (Camelus bactrianus), wild yak (Bos mutus), horse (Equus caballus), donkey (Equus asinus), reindeer (Rangifer tarandus), eurasian elk (Alces alces), alpaca (Vicugna pacos), zebu (Bos indicus), llama (Lama glama), or human (Homo sapiens). In some embodiments, a casein protein (e.g., α-S1 casein, α-S2 casein, β-casein, or κ-casein) has at least 80%, at least 85%, at least 90%, at least 95%, at least 96%, at least 97%, at least 98%, or at least 99% identity with a casein protein from one or more of cow (Bos taurus), goat (Capra hircus), sheep (Ovis aries), water buffalo (Bubalus bubalis), dromedary camel (Camelus dromedaries), bactrian camel (Camelus bactrianus), wild yak (Bos mutus), horse (Equus caballus), donkey (Equus asinus), reindeer (Rangifer tarandus), eurasian elk (Alces alces), alpaca (Vicugna pacos), zebu (Bos indicus), llama (Lama glama), or human (Homo sapiens).

As used herein, the term “α-S1 casein” refers to not only the α-S1 casein protein, but also fragments or variants thereof α-S1 casein is found in the milk of numerous different mammalian species, including cow, goat, and sheep. The sequence, structure and physical/chemical properties of α-S1 casein derived from various species is highly variable. An illustrative sequence for bovine α-S1 casein can be found at Uniprot Accession No. P02662, and an illustrative sequence for goat α-S1 casein can be found at GenBank Accession No. X59836.1. The terms “α-S1 casein” and “alpha-S1-casein” (and similar terms) are used interchangeably herein.

As used herein, the term “α-S2 casein” refers to not only the α-S2 casein protein, but also fragments or variants thereof α-S2 is known as epsilon-casein in mouse, Gamma-casein in rat, and casein-A in guinea pig. The sequence, structure and physical/chemical properties of α-S2 casein derived from various species is highly variable. An illustrative sequence for bovine α-S2 casein can be found at Uniprot Accession No. P02663, and an illustrative sequence for goat α-S2 casein can be found at Uniprot Accession No. P33049. The terms “α-S2 casein” and “alpha-S2-casein” (and similar terms) are used interchangeably herein.

As used herein, the term “β-casein” refers to not only the β-casein protein, but also fragments or variants thereof. For example, A1 and A2 β-casein are genetic variants of the β-casein milk protein that differ by one amino acid (at amino acid 67, A2 β-casein has a proline, whereas A1 has a histidine). Other genetic variants of β-casein include the A3, B, C, D, E, F, H1, H2, I and G genetic variants. The sequence, structure and physical/chemical properties of β-casein derived from various species is highly variable. Exemplary sequences for bovine β-casein can be found at Uniprot Accession No. P02666 and GenBank Accession No. MI5132.1. The terms “β-casein”, “beta-casein” and “B-casein” (and similar terms) are used interchangeably herein.

As used herein, the term “κ-casein” refers to not only the κ-casein protein, but also fragments or variants thereof. κ-casein is cleaved by rennet, which releases a macropeptide from the C-terminal region. The remaining product with the N-terminus and approximately two-thirds of the original peptide chain is referred to as para-κ-casein. The sequence, structure and physical/chemical properties of κ-casein derived from various species is highly variable. Illustrative sequences for bovine κ-casein can be found at Uniprot Accession No. P02668 and GenBank Accession No. CAA25231. The terms “κ-casein”, “κ-casein” and “kappa-casein” (and similar terms) are used interchangeably herein.

In some embodiments, the milk protein comprises from about: 75-85%, 80%-85%, 80%-90%, 85%-95%, 90%-95%, or 95%-100%, including all ranges and subranges therebetween, identity to a sequence selected from SEQ ID NO: 1-SEQ ID NO: 614. Provided milk proteins of the disclosure can have from about: 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, including all ranges and subranges therebetween to sequence provided and/or referenced in Table 17.

In some embodiments, the milk protein is a casein protein, for example, α-S1 casein, α-S2 casein, β-casein, and or κ-casein. In some embodiments, the milk protein is κ-casein and comprises the sequence of SEQ ID NO: 4, or a sequence at least 90%, at least 95%, at least 96%, at least 97%, at least 98%, or at least 99% identical thereto. In some embodiments, the milk protein is para-κ-casein and comprises the sequence of SEQ ID NO: 2, or a sequence at least 90%, at least 95%, at least 96%, at least 97%, at least 98%, or at least 99% identical thereto. In some embodiments, the milk protein is β-casein and comprises the sequence of SEQ ID NO: 6, or a sequence at least 90%, at least 95%, at least 96%, at least 97%, at least 98%, or at least 99% identical thereto. In some embodiments, the milk protein is α-S1 casein and comprises the sequence SEQ ID NO: 8, or a sequence at least 90%, at least 95%, at least 96%, at least 97%, at least 98%, or at least 99% identical thereto. In some embodiments, milk protein is α-S2 casein and comprises the sequence SEQ ID NO: 84, or a sequence at least 90%, at least 95%, at least 96%, at least 97%, at least 98%, or at least 99% identical thereto.

In some embodiments, the milk protein comprises a sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 4. In some embodiments, the milk protein comprises a sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 2. In some embodiments, the milk protein comprises a sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 6. In some embodiments, the milk protein comprises a sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 8. In some embodiments, the milk protein comprises a sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 84.

In some embodiments, α-S1 casein is encoded by the sequence of SEQ ID NO: 7, or a sequence at least 90%, at least 95%, at least 96%, at least 97%, at least 98%, or at least 99% identical thereto. In some embodiments, α-S2 casein is encoded by the sequence of SEQ ID NO: 83, or a sequence at least 90%, at least 95%, at least 96%, at least 97%, at least 98%, or at least 99% identical thereto. In some embodiments, β-casein is encoded by the sequence of SEQ ID NO: 5, or a sequence at least 90%, at least 95%, at least 96%, at least 97%, at least 98%, or at least 99% identical thereto. In some embodiments, κ-casein is encoded by the sequence of SEQ ID NO: 3, or a sequence at least 90%, at least 95%, at least 96%, at least 97%, at least 98%, or at least 99% identical thereto. In some embodiments, para-κ-casein is encoded by the sequence of SEQ ID NO: 1, or a sequence at least 90%, at least 95%, at least 96%, at least 97%, at least 98%, or at least 99% identical thereto.

In some embodiments, the milk protein is encoded by a sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 7. In some embodiments, the milk protein is encoded by a sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 83. In some embodiments, the milk protein is encoded by a sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 3. In some embodiments, the milk protein is encoded by a sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 1. In some embodiments, the milk protein is encoded by a sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 5.

In some embodiments, the milk protein is a casein protein, and comprises a sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to any one of SEQ ID NO: 85-133, or 148-563. In some embodiments, the milk protein is a casein protein and comprises the sequence of any one of SEQ ID NO: 85-133 or 148-563.

In some embodiments, the milk protein comprises a sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to any one of SEQ ID NO: 85-98 or 148-340. In some embodiments, the milk protein comprises the sequence of any one of SEQ ID NO: 85-98 or 148-340.

In some embodiments, the milk protein comprises a sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to any one of SEQ ID NO: 99-109 or 341-440. In some embodiments, the milk protein comprises the sequence of any one of SEQ ID NO: 99-109 or 341-440.

In some embodiments, the milk protein comprises a sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to any one of SEQ ID NO: 110-120 or 441-494. In some embodiments, the milk protein comprises the sequence of any one of SEQ ID NO: 110-120 or 441-494.

In some embodiments, the milk protein comprises a sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to any one of SEQ ID NO: 121-133 or 495-563. In some embodiments, the milk protein comprises the sequence of any one of SEQ ID NO: 121-133 or 495-563 or 495-563.

In some embodiments, the milk protein is not a casein protein. Examples of non-casein milk proteins include, for example, β-lactoglobulin, α-lactalbumin, lysozyme, lactoferrin, lactoperoxidase, serum albumin, or an immunoglobulin.

In some embodiments, a chordate protein comprises whey. As used herein “whey” refers to the liquid remaining after milk has been curdled and strained, for example during cheesemaking. Whey comprises a collection of globular proteins, typically a mixture of β-lactoglobulin, α-lactalbumin, bovine serum albumin, and immunoglobulins. The term “whey protein” may be used herein to refer to a milk protein which heat labile and is soluble in milk at about pH 4.6 in its undenatured state. Alpha-Lactalbumin (α-LA) and beta-lactoglobulin (β-LG) are the predominant whey proteins and comprise about 70-80% of the total whey proteins. Other types of whey proteins include, immunoglobulins (Igs) (e.g., IgA, IgG, IgM, IgE), serum albumin, lysozyme, lactoferrin (LF), lactoperoxidase (LP), and protease-peptones.

In some embodiments, the milk protein is a protein typically found in whey. In some embodiments, the milk protein is β-lactoglobulin or a functional fragment thereof. In some embodiments, the milk protein is β-lactoglobulin and comprises the sequence of SEQ ID NO: 10, or a sequence at least 90%, at least 95%, at least 96%, at least 97%, at least 98%, or at least 99% identical thereto. In some embodiments, the milk protein is β-lactoglobulin and is encoded by the sequence of any one of SEQ ID NO: 9, 11, 12, or 13, or a sequence at least 90%, at least 95%, at least 96%, at least 97%, at least 98%, or at least 99% identical to any one of SEQ ID NO: 9, 11, 12, or 13. In some embodiments, the milk protein comprises a sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to any one of SEQ ID NO: 9-13 or 564-614. In some embodiments, the milk protein comprises the sequence of any one of SEQ ID NO: 10 or 564-614.

In an aspect, a chordate protein is an egg protein. In some embodiments, an egg protein used in the compositions and methods described herein is an egg white protein. Egg white is made up of at least 40 different kinds of proteins. Ovalbumin is the major egg white protein, along with ovotransferrin and ovomucoid. Other proteins of interest include flavoprotein, which binds riboflavin; avidin, which can bind and inactivate biotin; and lysozyme, which has lytic action against bacteria.

In some embodiments, an egg protein used in the compositions and methods described herein is an egg yolk protein. Exemplary egg yolk proteins comprise: Phosvitins, vitellin, lipophorin, and combinations thereof.

In some embodiments, an egg protein is any one of: ovalbumin, ovotransferrin, ovomucoid, ovoglobulin G2, ovoglobulin G3, alpha-ovomucin, beta-ovomucin, lysozyme, ovoinhibitor, ovoglycoprotein, flavoprotein, ovomacroglobulin, avidin, cystatin, ovostatin, ovalbumin related protein X, ovalbumin related protein Y, vitellogenin, alpha-lipovitellin, beta-lipovitellin, alpha-livetin, beta-livetin, gamma-livetin, phosvitin, apovitellenin I, apovitellenin II, apovitellenin III, apovitellenin IV, apovitellenin V, apovitellenin VI, VLDL-II, apo-B, and any combination thereof. In other embodiments, an egg protein is selected from the group consisting of ovalbumin, ovotransferrin, ovomucoid, lysozyme, ovoglobulin G2, ovoglobulin G3, alpha-ovomucin, beta-ovomucin, apovitellenin-1, alpha-lipovitellin, beta-lipovitellin and any combination thereof. In some embodiments, an egg protein used in the DNA constructs described herein may include apolipoproteins, egg yolk globulin, or riboflavin binding protein. In some embodiments, a transgene construct described herein comprises a transgene encoding an ovalbumin protein. In some embodiments, an egg protein comprises a sequence or is encoded by a sequence selected from Table 6-Table 8. In some embodiments, an egg protein comprises a sequence or is encoded by a sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 100% identical to, including all ranges and subranges therebetween, a sequence selected from Table 6-Table 8.

In some embodiments, the chordate protein is ovalbumin or a functional fragment thereof. In some embodiments, the disclosure teaches ovalbumin protein sequence that is encoded by codon-optimized SEQ ID NO: 617. In a particular embodiment, the ovalbumin protein comprising an amino acid sequence having at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, sequence identity to SEQ ID NO:622 is provided. In some embodiments, the ovalbumin protein has the amino acid sequence of SEQ ID NO: 622.

In some embodiments, the disclosure teaches ovotransferrin protein sequence that is encoded by codon-optimized SEQ ID NO: 618. In a particular embodiment, the ovotransferrin protein comprising an amino acid sequence having at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, sequence identity to SEQ ID NO:623 is provided. In some embodiments, the ovotransferrin protein has the amino acid sequence of SEQ ID NO: 623.

In other embodiments, the disclosure teaches ovomucoid protein sequence that is encoded by codon-optimized SEQ ID NO: 619. In a particular embodiment, the ovomucoid protein comprising an amino acid sequence having at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, sequence identity to SEQ ID NO:624 is provided. In some embodiments, the ovomucoid protein has the amino acid sequence of SEQ ID NO: 624.

In other embodiments, the disclosure teaches lysozyme sequence that is encoded by codon-optimized SEQ ID NO: 620. In a particular embodiment, the lysozyme protein comprising an amino acid sequence having at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, sequence identity to SEQ ID NO:625 is provided. In some embodiments, the lysozyme protein has the amino acid sequence of SEQ ID NO: 625.

In other embodiments, the disclosure teaches apovitellenin-1 protein sequences that is encoded by codon-optimized SEQ ID NO: 621. In a particular embodiment, the apovitellenin-1 protein comprising an amino acid sequence having at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, sequence identity to SEQ ID NO:626 is provided. In some embodiments, the apovitellenin-1 protein has the amino acid sequence of SEQ ID NO: 626.

TABLE 6
Exemplary codon-optimized DNA sequences of egg proteins.

| Egg Protein | Sequence Identifier | Nucleotide Sequence |
|---|---|---|
| Ovalbumin | SEQ ID | ATGGGATCAATCGGT |
| | NO: 617 | GCTGCCAGCATGGAG |
| | | TTTTGCTTTGATGTAT |
| | | TTAAGGAACTCAAAG |
| | | TACATCACGCTAACG |
| | | AAAATATTTTCTACT |
| | | GTCCTATAGCTATAA |
| | | TGTCCGCACTTGCTAT |
| | | GGTCTATTTGGGCGC |
| | | CAAGGATTCTACACG |
| | | CACCCAGATTAATAA |
| | | GGTGGTTCGTTTTGA |
| | | CAAACTTCCAGGCTT |
| | | TGGTGATTCAATAGA |
| | | GGCCCAATGTGGGAC |
| | | AAGTGTCAACGTACA |
| | | CAGCTCTTTGCGTGA |
| | | TATACTCAACCAAAT |
| | | AACTAAACCCAATGA |
| | | CGTGTATAGTTTTTCC |
| | | CTTGCCTCCCGTCTTT |
| | | ATGCTGAAGAACGTT |
| | | ACCCAATATTGCCCG |
| | | AATACCTCCAATGTG |
| | | TCAAGGAACTGTATC |
| | | GCGGCGGACTTGAAC |
| | | CAATAAATTTTCAGA |
| | | CCGCAGCCGATCAGG |
| | | CCAGGGAGCTCATAA |
| | | ACTCATGGGTCGAAA |
| | | GTCAAACAAATGGGA |
| | | TCATACGTAATGTGC |
| | | TCCAGCCTTCTAGCG |
| | | TTGATTCACAAACCG |
| | | CCATGGTGTTGGTCA |
| | | ATGCCATCGTATTTA |
| | | AAGGTCTCTGGGAAA |
| | | AGACATTTAAGGATG |
| | | AAGATACTCAGGCAA |
| | | TGCCTTTCCGTGTAAC |
| | | CGAGCAGGAGTCCAA |
| | | ACCCGTTCAAATGAT |
| | | GTACCAGATAGGGTT |
| | | GTTTAGGGTAGCCAG |
| | | TATGGCCTCTGAGAA |
| | | AATGAAGATACTGGA |
| | | ATTGCCCTTTGCCAGT |
| | | GGTACCATGTCCATG |
| | | CTTGTACTGTTGCCA |
| | | GATGAAGTTTCTGGC |
| | | CTGGAGCAGCTTGAG |
| | | TCTATAATAAACTTC |
| | | GAGAAGTTGACAGAG |
| | | TGGACATCATCTAAC |
| | | GTTATGGAAGAACGT |
| | | AAAATAAAAGTGTAT |
| | | TTGCCTCGCATGAAG |
| | | ATGGAGGAGAAATAC |
| | | AACCTTACCAGTGTA |
| | | CTGATGGCAATGGGC |
| | | ATAACCGATGTTTTTT |
| | | CTAGTTCCGCAAACC |
| | | TTTCTGGTATCTCCTC |
| | | AGCAGAATCTCTGAA |
| | | GATATCCCAAGCAGT |
| | | TCATGCAGCACACGC |
| | | AGAAATAAACGAGGC |
| | | AGGACGTGAAGTGGT |
| | | AGGATCAGCCGAGGC |
| | | AGGCGTTGATGCAGC |
| | | ATCCGTGAGCGAAGA |
| | | GTTTCGTGCCGATCA |
| | | CCCTTTCCTTTTCTGC |
| | | ATCAAACACATTGCT |
| | | ACCAATGCCGTTCTTT |
| | | TCTTCGGGCGCTGTG |
| | | TATCCCCTTAA |
| Ovotransferrin | SEQ ID | ATGAAACTGATTTTG |
| | NO: 618 | TGTACCGTCCTGTCA |
| | | CTTGGGATTGCCGCA |
| | | GTATGTTTTGCCGCC |
| | | CCACCCAAGAGTGT |
| | | TATCCGTTGGTGTAC |
| | | CATCTCTAGCCCTGA |
| | | GGAGAAGAAATGTA |
| | | ACAACCTCAGGGAC |
| | | TTGACCCAGCAAGA |
| | | GAGGATAAGCCTGA |
| | | CATGTGTCCAGAAG |
| | | GCTACCTATCTCGAC |
| | | TGTATCAAAGCCAT |
| | | AGCCAACAACGAGG |
| | | CCGACGCAATATCC |
| | | CTTGATGGAGGACA |
| | | GGTGTTCGAGGCCG |
| | | GACTTGCCCCTTATA |
| | | AATTGAAGCCTATA |
| | | GCTGCTGAAATCTAC |
| | | GAGCATACTGAGGG |
| | | TTCTACCACTAGTTA |
| | | TTATGCCGTAGCCGT |
| | | AGTTAAAAAGGGGA |
| | | CCGAGTTTACAGTCA |
| | | ACGATCTCCAGGGT |
| | | AAAAATAGCTGCCA |
| | | TACTGGTCTTGGTAG |
| | | GAGTGCTGGGTGGA |
| | | ATATACCTATCGGTA |
| | | CACTCCTCCACTGGG |
| | | GCGCTATCGAGTGG |
| | | GAGGGTATCGAAAG |
| | | TGGAAGCGTGGAAC |
| | | AGGCAGTCGCTAAG |
| | | TTTTTTTCCGCCTCTT |
| | | GCGTACCCGGCGCT |
| | | ACTATAGAGCAGAA |
| | | ACTTTGCCGTCAGTG |
| | | CAAAGGAGATCCCA |
| | | AGACCAAGTGTGCC |
| | | CGTAACGCCCCCTAT |
| | | AGTGGATATTCCGG |
| | | CGCTTTCCACTGCTT |
| | | GAAGGATGGAAAGG |
| | | GGGACGTAGCCTTC |
| | | GTGAAACACACTAC |
| | | AGTGAATGAAAACG |
| | | CCCCCGATCTCAATG |
| | | ACGAGTATGAGTTG |
| | | CTTTGCCTGGATGGT |
| | | AGTCGCCAACCAGT |
| | | CGATAATTACAAAA |
| | | CCTGTAACTGGGCTC |
| | | GTGTTGCTGCACATG |
| | | CCGTCGTTGCACGCG |
| | | ATGACAATAAAGTG |
| | | GAGGATATCTGGTC |
| | | CTTTCTGTCAAAAGC |
| | | CCAAAGCGATTTTG |
| | | GCGTAGATACCAAG |
| | | TCAGATTTCCATCTG |
| | | TTTGGGCCACCTGGA |
| | | AAGAAAGACCCTGT |
| | | GCTTAAGGACTTCTT |
| | | GTTCAAAGACAGTG |
| | | CCATAATGCTCAAA |
| | | CGCGTTCCTAGCCTT |
| | | ATGGATTCTCAACTG |
| | | TATCTCGGGTTCGAA |
| | | TATTACTCAGCCATA |
| | | CAATCAATGAGGAA |
| | | GGACCAGCTCACTC |
| | | CTAGCCCTAGAGAG |
| | | AATAGAATTCAATG |
| | | GTGTGCTGTTGGAA |
| | | AAGACGAGAAATCT |
| | | AAATGCGACCGTTG |
| | | GAGCGTCGTCAGCA |
| | | ACGGTGATGTTGAG |
| | | TGTACTGTAGTTGAT |
| | | GAAACTAAGGATTG |
| | | CATTATTAAGATTAT |
| | | GAAGGGGGAGGCCG |
| | | ATGCCGTCGCATTGG |
| | | ATGGAGGGCTGGTT |
| | | TATACCGCAGGTGTC |
| | | TGTGGACTGGTGCCT |
| | | GTCATGGCAGAAAG |
| | | ATATGATGATGAAT |
| | | CACAATGCAGTAAA |
| | | ACAGACGAACGTCC |
| | | AGCCTCATATTTTGC |
| | | TGTCGCCGTCGCCCG |
| | | CAAGGATTCCAATG |
| | | TGAATTGGAATAATT |
| | | TGAAGGGAAAAAAA |
| | | TCCTGCCACACTGCT |
| | | GTAGGGCGTACAGC |
| | | AGGTTGGGTGATCC |
| | | CAATGGGACTGATT |
| | | CACAATAGGACCGG |
| | | TACTTGCAACTTTGA |
| | | TGAATATTTCTCCGA |
| | | GGGGTGTGCTCCCG |
| | | GTAGCCCCCCCAAC |
| | | AGCCGCTTGTGCCA |
| | | GCTGTGCCAAGGTA |
| | | GTGGAGGTATTCCCC |
| | | CTGAGAAATGCGTC |
| | | GCTTCCTCTCACGAG |
| | | AAATACTTTGGTTAT |
| | | ACTGGTGCCTTGCGT |
| | | TGCCTCGTAGAAAA |
| | | AGGTGACGTCGCTTT |
| | | TATCCAACACAGTA |
| | | CTGTAGAGGAGAAT |
| | | ACAGGGGGGAAGAA |
| | | CAAAGCTGACTGGG |
| | | CTAAGAATCTTCAG |
| | | ATGGACGATTTCGA |
| | | ACTGTTGTGTACCGA |
| | | TGGTAGAAGGGCTA |
| | | ATGTTATGGATTATC |
| | | GTGAGTGCAATCTTG |
| | | CAGAAGTACCAACC |
| | | CATGCTGTAGTTGTC |
| | | AGACCCGAGAAAGC |
| | | AAATAAAATTAGAG |
| | | ACCTTTTGGAGAGA |
| | | CAAGAGAAACGTTT |
| | | CGGTGTAAACGGCT |
| | | CAGAGAAGTCTAAA |
| | | TTCATGATGTTTGAA |
| | | AGTCAAAATAAGGA |
| | | TCTGTTGTTCAAAGA |
| | | CTTGACTAAATGCCT |
| | | GTTTAAAGTCAGGG |
| | | AAGGCACCACTTAT |
| | | AAAGAATTTCTGGG |
| | | AGATAAATTCTACA |
| | | CAGTAATAAGCAAC |
| | | TTGAAAACATGCAA |
| | | TCCTTCCGATATCCT |
| | | GCAAATGTGCAGTTT |
| | | CCTTGAAGGGAAAT |
| | | AA |
| Ovomucoid | SEQ ID | ATGGCTATGGCCGGA |
| | NO: 619 | GTCTTTGTTCTTTTCT |
| | | CTTTCGTGCTTTGTGG |
| | | TTTTTTGCCTGACGCC |
| | | GCCTTCGGAGCCGAG |
| | | GTTGACTGCTCTCGCT |
| | | TCCCAAATGCAACCG |
| | | ATAAAGAGGGAAAG |
| | | GACGTTCTTGTTTGCA |
| | | ACAAGGACCTGCGTC |
| | | CTATCTGTGGGACAG |
| | | ATGGCGTTACATATA |
| | | CTAACGACTGCCTTTT |
| | | GTGTGCATATTCTATT |
| | | GAATTTGGTACTAAC |
| | | ATATCTAAGGAGCAT |
| | | GATGGGGAATGCAAA |
| | | GAAACAGTTCCCATG |
| | | AACTGTAGTTCTTAT |
| | | GCTAATACCACTAGT |
| | | GAGGACGGCAAGGTC |
| | | ATGGTATTGTGCAAT |
| | | AGGGCTTTCAATCCT |
| | | GTATGCGGGACAGAT |
| | | GGTGTCACTTACGAC |
| | | AATGAATGTTTGCTG |
| | | TGTGCTCACAAGGTC |
| | | GAACAAGGAGCTTCT |
| | | GTCGATAAGAGGCAT |
| | | GATGGGGGTTGCAGA |
| | | AAAGAATTGGCTGCA |
| | | GTATCAGTTGACTGC |
| | | TCTGAGTATCCCAAG |
| | | CCAGATTGCACCGCC |
| | | GAGGACCGCCCATTG |
| | | TGTGGAAGCGATAAT |
| | | AAGACTTATGGAAAT |
| | | AAATGCAACTTCTGC |
| | | AATGCCGTTGTGGAA |
| Lysozyme | SEQ ID | ATGAGGAGCTTGTT |
| | NO: 620 | GATACTTGTGCTTTG |
| | | TTTCCTTCCCCTTGC |
| | | AGCATTGGGGAAAG |
| | | TTTTCGGTAGGTGCG |
| | | AGCTTGCCGCCGCA |
| | | ATGAAAAGACACGG |
| | | CTTGGACAATTATCG |
| | | TGGATACTCTCTCGG |
| | | CAATTGGGTTTGTGT |
| | | AGCAAAGTTCGAGA |
| | | GCAATTTTAACACCC |
| | | AGGCTACTAATAGA |
| | | AATACCGACGGATC |
| | | TACCGACTACGGGA |
| | | TTCTGCAAATAAAC |
| | | AGCCGCTGGTGGTG |
| | | TAATGACGGGCGTA |
| | | CTCCCGGTAGCCGC |
| | | AATCTCTGTAACATC |
| | | CCCTGTAGTGCATTG |
| | | CTTAGTTCTGACATT |
| | | ACAGCTAGCGTGAA |
| | | CTGTGCTAAAAAGA |
| | | TAGTTTCTGACGGTA |
| | | ATGGAATGAGTGCT |
| | | TGGGTTGCCTGGAG |
| | | GAACCGTTGTAAGG |
| | | GGACCGACGTTCAA |
| | | GCATGGATTAGAGG |
| | | GTGTCGTCTGTGA |
| Apovitellenin-1 | SEQ ID | AGCAACGGCACTCTG |
| | NO: 621 | ACTTTGAGTCATTTCG |
| | | GAAAATGCTGA |
| | | ATGGTACAATACAGA |
| | | GCACTCGTGATTGCC |
| | | GTAATTTTGCTTCTTT |
| | | CCACTACCGTCCCTG |
| | | AGGTACATAGCAAGT |
| | | CCATCATTGACAGAG |
| | | AACGCAGGGACTGGC |
| | | TGGTGATTCCTGATG |
| | | CTGCTGCTGCCTATAT |
| | | TTATGAAGCCGTCAA |
| | | CAAGGTATCACCACG |
| | | CGCAGGTCAGTTCTT |
| | | GCTCGACGTTTCTCA |
| | | AACCACAGTCGTGTC |
| | | TGGAATCAGGAACTT |
| | | TCTCATCAACGAAAC |
| | | AGCTAGGCTTACTAA |
| | | GCTGGCCGAGCAACT |
| | | TATGGAGAAAATTAA |
| | | GAACCTTTGCTATAC |
| | | TAAAGTGTTGGGCTA |
| | | CTAG |

TABLE 7
Exemplary protein sequences of egg proteins which are a translated
version of the codon-optimized nucleotide sequences of Table 6.

| | | Protein Sequence |
|---|---|---|
| Protein Name | Sequence Identifier | (Amino Acid) |
| Ovalbumin | SEQ ID NO: 622 | MGSIGAASMEFCFDV |
| | | FKELKVHHANENIFY |
| | | CPIAIMSALAMVYLG |
| | | AKDSTRTQINKVVRF |
| | | DKLPGFGDSIEAQCG |
| | | TSVNVHSSLRDILNQI |
| | | TKPNDVYSFSLASRL |
| | | YAEERYPILPEYLQCV |
| | | KELYRGGLEPINFQT |
| | | AADQARELINSWVES |
| | | QINGIIRNVLQPSSVD |
| | | SQTAMVLVNAIVFKG |
| | | LWEKTFKDEDTQAM |
| | | PFRVTEQESKPVQMM |
| | | YQIGLFRVASMASEK |
| | | MKILELPFASGTMSM |
| | | LVLLPDEVSGLEQLES |
| | | INFEKLTEWTSSNVM |
| | | EERKIKVYLPRMKME |
| | | EKYNLTSVLMAMGIT |
| | | DVFSSSANLSGISSAE |
| | | SLKISQAVHAAHAEI |
| | | NEAGREVVGSAEAG |
| | | VDAASVSEEFRADHP |
| | | FLFCIKHIATNAVLFF |
| | | GRCVSP |
| Ovotransferrin | SEQ ID NO: 623 | MKLILCTVLSLGIAAV |
| | | CFAAPPKSVIRWCTISS |
| | | PEEKKCNNLRDLTQQE |
| | | RISLTCVQKATYLDCI |
| | | KAIANNEADAISLDGG |
| | | QVFEAGLAPYKLKPIA |
| | | AEIYEHTEGSTTSYYA |
| | | VAVVKKGTEFTVNDL |
| | | QGKNSCHTGLGRSAG |
| | | WNIPIGTLLHWGAIEW |
| | | EGIESGSVEQAVAKFF |
| | | SASCVPGATIEQKLCR |
| | | QCKGDPKTKCARNAP |
| | | YSGYSGAFHCLKDGK |
| | | GDVAFVKHTTVNENA |
| | | PDLNDEYELLCLDGSR |
| | | QPVDNYKTCNWARV |
| | | AAHAVVARDDNKVE |
| | | DIWSFLSKAQSDFGVD |
| | | TKSDFHLFGPPGKKDP |
| | | VLKDFLFKDSAIMLKR |
| | | VPSLMDSQLYLGFEYY |
| | | SAIQSMRKDQLTPSPR |
| | | ENRIQWCAVGKDEKS |
| | | KCDRWSVVSNGDVEC |
| | | TVVDETKDCIIKIMKG |
| | | EADAVALDGGLVYTA |
| | | GVCGLVPVMAERYDD |
| | | ESQCSKTDERPASYFA |
| | | VAVARKDSNVNWNN |
| | | LKGKKSCHTAVGRTA |
| | | GWVIPMGLIHNRTGTC |
| | | NEDEYFSEGCAPGSPP |
| | | NSRLCQLCQGSGGIPP |
| | | EKCVASSHEKYFGYT |
| | | GALRCLVEKGDVAFIQ |
| | | HSTVEENTGGKNKAD |
| | | WAKNLQMDDFELLCT |
| | | DGRRANVMDYRECNL |
| | | AEVPTHAVVVRPEKA |
| | | NKIRDLLERQEKRFGV |
| | | NGSEKSKFMMFESQN |
| | | KDLLFKDLTKCLFKVR |
| | | EGTTYKEFLODKFYTV |
| | | ISNLKTCNPSDILQMCS |
| | | FLEGK |
| Ovomucoid | SEQ ID NO: 624 | MAMAGVFVLFSFVL |
| | | CGFLPDAAFGAEVDC |
| | | SRFPNATDKEGKDVL |
| | | VCNKDLRPICGTDGV |
| | | TYTNDCLLCAYSIEFG |
| | | TNISKEHDGECKETV |
| | | PMNCSSYANTTSEDG |
| | | KVMVLCNRAFNPVC |
| | | GTDGVTYDNECLLCA |
| | | HKVEQGASVDKRHD |
| | | GGCRKELAAVSVDCS |
| | | EYPKPDCTAEDRPLC |
| | | GSDNKTYGNKCNFC |
| | | NAVVESNGTLTLSHF |
| | | GKC |
| Lysozyme | SEQ ID NO: 625 | MRSLLILVLCFLPLAA |
| | | LGKVFGRCELAAAMK |
| | | RHGLDNYRGYSLGNW |
| | | VCVAKFESNENTQAT |
| | | NRNTDGSTDYGILQIN |
| | | SRWWCNDGRTPGSRN |
| | | LCNIPCSALLSSDITAS |
| | | VNCAKKIVSDGNGMS |
| | | AWVAWRNRCKGTDV |
| | | QAWIRGCRL |
| Apovitellenin-1 | SEQ IDN O: 626 | MVQYRALVIAVILLL |
| | | STTVPEVHSKSHIDRE |
| | | RRDWL VIPDAAAA YI |
| | | YEAVNKVSPRAGQFL |
| | | LDVSQTTVVSGIRNFL |
| | | INETARLTKLAEQLM |
| | | EKIKNLCYTKVLGY |

TABLE 8
Additional exemplary ovalbumin protein sequences of the disclosure

| SEQ | | Species | | |
|---|---|---|---|---|
| ID | | (Common | Accession | Protein Sequence |
| NO | Description | Name) | Number | (Amino Acid) |
| 810 | Ovalbumin | Meleagris | XP_010706723.1 | MGSIGAVSMEFCFDVFKELK |
| | | gallopavo | | VHHANENIFYSPFTIISALA |
| | | (wild turkey) | | MVYLGAKDSTRTQINKVVRF |
| | | | | DKLPGFGDSVEAQCGTSVNV |
| | | | | HSSLRDILNQITKPNDVYSF |
| | | | | SLASRLYAEETYPILPEYLQ |
| | | | | CVKELYRGGLESINFQTAAD |
| | | | | QARGLINSWVESQTNGMIKN |
| | | | | VLQPSSVDSQTAMVLVNAIV |
| | | | | FKGLWEKAFKDEDTQAIPFR |
| | | | | VTEQESKPVQMMYQIGLFKV |
| | | | | ASMASEKMKILELPFASGTM |
| | | | | SMWVLLPDEVSGLEQLETTI |
| | | | | SFEKMTEWISSNIMEERRIK |
| | | | | VYLPRMKMEEKYNLTSVLMA |
| | | | | MGITDLFSSSANLSGISSAG |
| | | | | SLKISQAVHAAYAEIYEAGR |
| | | | | EVIGSAEAGADATSVSEEFR |
| | | | | VDHPFLYCIKHNLTNSILFF |
| | | | | GRCISP |
| 811 | | | NP_001290119.1 | MGSIGAVSMEFCFDVFKELK |
| | | | | VHHANENIFYSPFTIISALA |
| | | | | MVYLGAKDSTRTQINKVVRF |
| | | | | DKLPGFGDSVEAQCGTSVNV |
| | | | | HSSLRDILNQITKPNDVYSF |
| | | | | SLASRLYAEETYPILPEYLQ |
| | | | | CVKELYRGGLESINFQTAAD |
| | | | | QARGLINSWVESQTNGMIKN |
| | | | | VLQPSSVDSQTAMVLVNAIV |
| | | | | FKGLWEKAFKDEDTQAIPFR |
| | | | | VTEQESKPVQMMYQIGLFKV |
| | | | | ASMASEKMKILELPFASGTM |
| | | | | SMWVLLPDEVSGLEQLETTI |
| | | | | SFEKMTEWISSNIMEERRIK |
| | | | | VYLPRMKMEEKYNLTSVLMA |
| | | | | MGITDLFSSSANLSGISSAG |
| | | | | SLKISQAAHAAYAEIYEAGR |
| | | | | EVIGSAEAGADATSVSEEFR |
| | | | | VDHPFLYCIKHNLTNSILFF |
| | | | | GRCISP |
| 812 | Ovalbumin | Coturnix japonica | P19104.2 | MGSIGAASMEFCFDVFKELK |
| | | (Japanese quail) | | VHHANDNMLYSPFAILSTLA |
| | | | | MVFLGAKDSTRTQINKVVHF |
| | | | | DKLPGFGDSIEAQCGTSVNV |
| | | | | HSSLRDILNQITKQNDAYSF |
| | | | | SLASRLYAQETYTVVPEYLQ |
| | | | | CVKELYRGGLESVNFQTAAD |
| | | | | QARGLINAWVESQTNGIIRN |
| | | | | ILQPSSVDSQTAMVLVNAIA |
| | | | | FKGLWEKAFKAEDTQTIPFR |
| | | | | VTEQESKPVQMMYQIGSFKV |
| | | | | ASMASEKMKILELPFASGTM |
| | | | | SMLVLLPDDVSGLEQLESII |
| | | | | SFEKLTEWTSSSIMEERKVK |
| | | | | VYLPRMKMEEKYNLTSLLMA |
| | | | | MGITDLFSSSANLSGISSVG |
| | | | | SLKISQAVHAAHAEINEAGR |
| | | | | DVVGSAEAGVDATEEFRADH |
| | | | | PFLFCVKHIETNAILLFGRC |
| | | | | VSP |
| 813 | Ovalbumin | Bambusicola | POI27989.1 | YYRVPCMVLCTAFHPYIFIV |
| | | thoracicus | | LLFALDNSEFTMGSIGAVSM |
| | | (Chinese bamboo | | EFCFDVFKELRVHHPNENIF |
| | | partridge) | | FCPFAIMSAMAMVYLGAKDS |
| | | | | TRTQINKVIRFDKLPGFGDS |
| | | | | TEAQCGKSANVHSSLKDILN |
| | | | | QITKPNDVYSFSLASRLYAD |
| | | | | ETYSIQSEYLQCVNELYRGG |
| | | | | LESINFQTAADQARELINSW |
| | | | | VESQINGIIRNVLQPSSVDS |
| | | | | QTAMVLVNAIVFRGLWEKAF |
| | | | | KDEDTQTMPFRVTEQESKPV |
| | | | | QMMYQIGSFKVASMASEKMK |
| | | | | ILELPLASGTMSMLVLLPDE |
| | | | | VSGLEQLETTISFEKLTEWT |
| | | | | SSNVMEERKIKVYLPRMKME |
| | | | | EKYNLTSVLMAMGITDLFRS |
| | | | | SANLSGISLAGNLKISQAVH |
| | | | | AAHAEINEAGRKAVSSAEAG |
| | | | | VDATSVSEEFRADRPFLFCI |
| | | | | KHIATKVVFFFGRYTSP |
| 814 | Ovalbumin | Numida | XP_021241976.1 | MASIGAVSTEFCVDVYKELR |
| | | Meleagris | | VHHANENIFYSPFTIISTLA |
| | | (Helmeted | | MVYLGAKDSTRTQINKVVRF |
| | | guineafowl) | | DKLPGFGDSIEAQCGTSVNV |
| | | | | HSSLRDILNQITKPNDVYSF |
| | | | | SLASRLYAEETYPILPEYLQ |
| | | | | CVKELYRGGLESINFQTAAD |
| | | | | QARELINSWVESQTSGIIKN |
| | | | | VLQPSSVNSQTAMVLVNAIY |
| | | | | FKGLWERAFKDEDTQAIPFR |
| | | | | VTEQESKPVQMMSQIGSFKV |
| | | | | ASVASEKVKILELPFVSGTM |
| | | | | SMLVLLPDEVSGLEQLESTI |
| | | | | STEKLTEWTSSSIMEERKIK |
| | | | | VFLPRMRMEEKYNLTSVLMA |
| | | | | MGMTDLFSSSANLSGISSAE |
| | | | | SLKISQAVHAAYAEIYEAGR |
| | | | | EVVSSAEAGVDATSVSEEFR |
| | | | | VDHPFLLCIKHNPTNSILFF |
| | | | | GRCISP |
| 815 | | | XP_021241975.1 | MALCKAFHPYIFIVLLFDVD |
| | | | | NSAFTMASIGAVSTEFCVDV |
| | | | | YKELRVHHANENIFYSPFTI |
| | | | | ISTLAMVYLGAKDSTRTQIN |
| | | | | KVVRFDKLPGFGDSIEAQCG |
| | | | | TSVNVHSSLRDILNQITKPN |
| | | | | DVYSFSLASRLYAEETYPIL |
| | | | | PEYLQCVKELYRGGLESINF |
| | | | | QTAADQARELINSWVESQTS |
| | | | | GIIKNVLQPSSVNSQTAMVL |
| | | | | VNAIYFKGLWERAFKDEDTQ |
| | | | | AIPFRVTEQESKPVQMMSQI |
| | | | | GSFKVASVASEKVKILELPF |
| | | | | VSGTMSMLVLLPDEVSGLEQ |
| | | | | LESTISTEKLTEWTSSSIME |
| | | | | ERKIKVFLPRMRMEEKYNLT |
| | | | | SVLMAMGMTDLFSSSANLSG |
| | | | | ISSAESLKISQAVHAAYAEI |
| | | | | YEAGREVVSSAEAGVDATSV |
| | | | | SEEFRVDHPFLLCIKHNPTN |
| | | | | SILFFGRCISP |
| 816 | Ovalbumin | Odontophorus | NXJ07552.1 | RILCMAFHPYIFIVLLFAPD |
| | | gujanensis | | NSEFTMGSIGAVSTEFCFDV |
| | | (Marbled wood | | FKELKVHHANENIFYSPFTI |
| | | quail) | | ISALAMVYLGAKDSTRTQIN |
| | | | | KVVRFDKLPGFGDSIEAQCG |
| | | | | TSVNVHSSLRDILNQITKPN |
| | | | | DFYSFSLASRLYADEAYPIL |
| | | | | PEYLQCVKELYRGGLESINF |
| | | | | QTAADQARELINSWVESQTS |
| | | | | GIIRNVLQPSSVDSQTAIVL |
| | | | | VNAIYFKALWKKGFKNEDTQ |
| | | | | AIPFRVTEQESKSVQMMQQI |
| | | | | GTFKVASVASEKMKILELPF |
| | | | | ASGTMSMWVLLPDEVSDLEQ |
| | | | | LETTISFEKLTEWTSSNIME |
| | | | | ERKIKVFLPRMKMEEKYNLT |
| | | | | SVLMAMGMTDLFSSSANLSG |
| | | | | ISSAESLKISQAVHAAYAEI |
| | | | | YEAGSEVVGSAEAGVDATSA |
| | | | | TEEFRVDRPFLFCIKHNPTN |
| | | | | SILFFGRCISP |
| 817 | Ovalbumin | Coturnix | XP_015709965.1 | MGSIGAASMEFCFDVFKELK |
| | | japonica | | VHHANDNMLYSPFAILSTLA |
| | | (Japanese quail) | | MVFLGAKDSTRTQINKVVHF |
| | | | | DKLPGFGDSIEAQCGTSANV |
| | | | | HSSLRDILNQITKQNDAYSF |
| | | | | SLASRLYAQETYTVVPEYLQ |
| | | | | CVKELYRGGLESVNFQTAAD |
| | | | | QARGLINAWVESQINGIIRN |
| | | | | ILQPSSVDSQTAMVLVNAIA |
| | | | | FKGLWEKAFKAEDTQTIPFR |
| | | | | VTEQESKPVQMMHQIGSFKV |
| | | | | ASMASEKMKILELPFASGTM |
| | | | | SMLVLLPDDVSGLEQLESTI |
| | | | | SFEKLTEWTSSSIMEERKVK |
| | | | | VYLPRMKMEEKYNLTSLLMA |
| | | | | MGITDLFSSSANLSGISSVG |
| | | | | SLKISQAVHAAYAEINEAGR |
| | | | | DVVGSAEAGVDATEEFRADH |
| | | | | PFLFCVKHIETNAILLFGRC |
| | | | | VSP |
| 818 | | | XP_015709964.1 | MGLCTAFHPYIFIVLLFALD |
| | | | | NSEFTMGSIGAASMEFCFDV |
| | | | | FKELKVHHANDNMLYSPFAI |
| | | | | LSTLAMVFLGAKDSTRTQIN |
| | | | | KVVHFDKLPGFGDSIEAQCG |
| | | | | TSANVHSSLRDILNQITKQN |
| | | | | DAYSFSLASRLYAQETYTVV |
| | | | | PEYLQCVKELYRGGLESVNF |
| | | | | QTAADQARGLINAWVESQTN |
| | | | | GIIRNILQPSSVDSQTAMVL |
| | | | | VNAIAFKGLWEKAFKAEDTQ |
| | | | | TIPFRVTEQESKPVQMMHQI |
| | | | | GSFKVASMASEKMKILELPF |
| | | | | ASGTMSMLVLLPDDVSGLEQ |
| | | | | LESTISFEKLTEWTSSSIME |
| | | | | ERKVKVYLPRMKMEEKYNLT |
| | | | | SLLMAMGITDLFSSSANLSG |
| | | | | ISSVGSLKISQAVHAAYAEI |
| | | | | NEAGRDVVGSAEAGVDATEE |
| | | | | FRADHPFLFCVKHIETNAIL |
| | | | | LFGRCVSP |
| 819 | Ovalbumin | Coturnix coturnix | Q6V115.3 | MGSIGAASMEFCFDVFKELK |
| | | (European quail) | | VHHANDNMLYSPFAILSTLA |
| | | | | MVFLGAKDSTRTQINKVVHF |
| | | | | DKLPGFGDSIEAQCGTSANV |
| | | | | HSSLRDILNQITKQNDAYSF |
| | | | | SLASRLYAQETYTVVPEYLQ |
| | | | | CVKELYRGGLESVNFQTAAD |
| | | | | QARGLINAWVESQINGIIRN |
| | | | | ILQPSSVDSQTAMVLVNAIA |
| | | | | FKGLWEKAFKAEDTQTIPFR |
| | | | | VTEQESKPVQMMHQIGSFKV |
| | | | | ASMASEKMKILELPFASGTM |
| | | | | SMLVLLPDDVSGLEQLESTI |
| | | | | SFEKLTEWTSSSIMEERKVK |
| | | | | VYLPRMKMEEKYNLTSLLMA |
| | | | | MGITDLFSSSANLSGISSVG |
| | | | | SLKIPQAVHAAYAEINEAGR |
| | | | | DVVGSAEAGVDATEEFRADH |
| | | | | PFLFCVKHIETNAILLFGRC |
| | | | | VSP |
| 820 | Ovalbumin | Phasianus | XP_031445133.1 | MGSIGAVSMEFCFDVLKELK |
| | | colchicus | | VHHANENYFYAPFTMFSALA |
| | | (Pheasant) | | MIYLGAKDSTRAQINKVVRF |
| | | | | DKLPGFGDSIEAQCGTSADP |
| | | | | QVHSSLRDILNQITKPNDAY |
| | | | | SFSLASRLYADEKYSIVPEY |
| | | | | LKCVKELYRGDVESINFQTA |
| | | | | ADQARGLINSWVESQTNGMI |
| | | | | KNVLQPSSVDSQTAMVLVNA |
| | | | | VVFKGLWEKAFKEEDTQAIP |
| | | | | FRVTEQESKPVQMMHQIGLF |
| | | | | KVASVPSEKMKILELPFASG |
| | | | | TMSMWVLLPDEVSGLEQLET |
| | | | | TISFEKMTEWTSSNIMEERK |
| | | | | IRVYLPRMKMEEKYNLTSIL |
| | | | | MAMGMTDLFSSSANLSGISS |
| | | | | VGSLKISQAVHAAYAEIYEA |
| | | | | GREVAGSAEAAMDATSVSEE |
| | | | | FRVDHPFLYCIKHNPSNTLL |
| | | | | FLGRCIFP |
| 821 | | | XP_031445132.1 | MALCTAFHPYVFIILLFALD |
| | | | | NSEFTMGSIGAVSMEFCFDV |
| | | | | LKELKVHHANENYFYAPFTM |
| | | | | FSALAMIYLGAKDSTRAQIN |
| | | | | KVVRFDKLPGFGDSIEAQCG |
| | | | | TSADPQVHSSLRDILNQITK |
| | | | | PNDAYSFSLASRLYADEKYS |
| | | | | IVPEYLKCVKELYRGDVESI |
| | | | | NFQTAADQARGLINSWVESQ |
| | | | | TNGMIKNVLQPSSVDSQTAM |
| | | | | VLVNAVVFKGLWEKAFKEED |
| | | | | TQAIPFRVTEQESKPVQMMH |
| | | | | QIGLFKVASVPSEKMKILEL |
| | | | | PFASGTMSMWVLLPDEVSGL |
| | | | | EQLETTISFEKMTEWTSSNI |
| | | | | MEERKIRVYLPRMKMEEKYN |
| | | | | LTSILMAMGMTDLFSSSANL |
| | | | | SGISSVGSLKISQAVHAAYA |
| | | | | EIYEAGREVAGSAEAAMDAT |
| | | | | SVSEEFRVDHPFLYCIKHNP |
| | | | | SNTLLFLGRCIFP |
| 822 | Ovalbumin | Penelope pileate | NXC49292.1 | IALRTAYPPYIVIVLLFALD |
| | | (White-crested | | NSEFTMASIGAVSTEFCFNV |
| | | guan) | | FRELKVQHANENIFYCPFTI |
| | | | | FSALAFAYLGAKENTRTQIN |
| | | | | KVAHFDKLPGFGDSIEAQCG |
| | | | | TSANVHSSLRDILNQITKPS |
| | | | | DNYSLSLASRLYVDERYPIL |
| | | | | PEYLQCVKELYRGGVEPITF |
| | | | | QTAADQARELINSWVESQTN |
| | | | | GMIKNILQPSSVDSQTAMVL |
| | | | | VNAVYFKGMWQKAFKNEDTQ |
| | | | | EMPFRITENESKPVQMMHQI |
| | | | | GSFKIATVASEKLKILELPY |
| | | | | ASGMMSMLVLLPDQASGLEQ |
| | | | | LENTISFEKLNEWTSSNMVE |
| | | | | ERRIKVYLPRMKMEEKYNLT |
| | | | | AVLTALGITDLFSPSANLSG |
| | | | | ISSAASLKISQAVHAAYAEI |
| | | | | YEAGRDVVGSAEAGVDATSV |
| | | | | TDEFRVDHPFLFCMKHNPSN |
| | | | | SIVFLGKCVSP |
| 823 | Ovalbumin | Anseranas | NXI67304.1 | CTAFHHYIVIVLLLFALDNS |
| | | semipalmata | | DFTMGSIGAASAEFCFDVFK |
| | | (Magpie goose) | | ELKVHHANENICYSPLSIIS |
| | | | | ALAMVYLGARDNTRTQIDKV |
| | | | | VHFDQIPGFGESIESQCGTS |
| | | | | VSVHSSLTDILTQITKPSDN |
| | | | | YSFSLASRLYAEETYPILPE |
| | | | | YLQCVKELYKGGLESISFQT |
| | | | | AADQARELINSWVESQTNGI |
| | | | | IKNILQPSSVDSQTAMVLVN |
| | | | | AIYFKGMWEKAFKDENTQEM |
| | | | | PFRVTEQESKPVQMMFQFGS |
| | | | | FKVATVASEKVKILELPYAS |
| | | | | GMISMCVLLPDEVSGLEQIE |
| | | | | NTISLEKLTEWTSSNMMEER |
| | | | | RMKVYLPRMKLEEQYNLTSV |
| | | | | LMALGMTDLFSPSANLSGIS |
| | | | | SAESLKISEAVHAAYVEIYE |
| | | | | AGREVVGSAEAGMDVSSVSE |
| | | | | EFRVDHPFLFLIKHNPSNSI |
| | | | | LFFGRLISP |
| 824 | Ovalbumin | Chauna torquata | NXK52213.1 | HYVCTAFHHHTVIVLLLFAL |
| | | (Southern | | DNSDFTMGSIGAASTEFCFD |
| | | screamer) | | VFKELKVQHVNGNIFYSPLS |
| | | | | IISALAMVYLGARDNTRTQI |
| | | | | DKVVHFDKIPGFGESIEAQC |
| | | | | GTSESVHSSLKDILTQITKP |
| | | | | SDNFSLSLASRLYAEETYPI |
| | | | | LPEYLQCVKELYKGGLESVS |
| | | | | FQTAADQARELISSWVESQT |
| | | | | NGIIKNILQPSSVDSQTEMV |
| | | | | LVNAIYFKGMWEKAFKDEDT |
| | | | | QTMPFRITEQESKPMQMMYQ |
| | | | | VGSFKVAVVASEKMKILELP |
| | | | | YASGMMSMWVLLPDEVSGLE |
| | | | | QLETTISFEKLTEWTSSNMM |
| | | | | EERRMKVYLPRMKMEEKYNL |
| | | | | TSVLIALGMTDLFSSSANLS |
| | | | | GISSAESLKMSEAVHAAYVE |
| | | | | IYEAGREVVGSAEAGMDVTS |
| | | | | VSEEFKADRPFLFLIKHNPT |
| | | | | NSILFFGRWISP |
| 825 | Ovalbumin | Anas | NP_001298098.1 | MGSIGAASTEFCFDVFRELR |
| | | platyrhynchos | | VQHVNENIFYSPFSIISALA |
| | | (Mallard) | | MVYLGARDNTRTQIDKVVHF |
| | | | | DKLPGFGESMEAQCGTSVSV |
| | | | | HSSLRDILTQITKPSDNFSL |
| | | | | SFASRLYAEETYAILPEYLQ |
| | | | | CVKELYKGGLESISFQTAAD |
| | | | | QARELINSWVESQTNGIIKN |
| | | | | ILQPSSVDSQTTMVLVNAIY |
| | | | | FKGMWEKAFKDEDTQAMPFR |
| | | | | MTEQESKPVQMMYQVGSFKV |
| | | | | AMVTSEKMKILELPFASGMM |
| | | | | SMFVLLPDEVSGLEQLESTI |
| | | | | SFEKLTEWTSSTMMEERRMK |
| | | | | VYLPRMKMEEKYNLTSVFMA |
| | | | | LGMTDLFSSSANMSGISSTV |
| | | | | SLKMSEAVHAACVEIFEAGR |
| | | | | DVVGSAEAGMDVTSVSEEFR |
| | | | | ADHPFLFFIKHNPTNSILFF |
| | | | | GRWMSP |
| 826 | | | XP_038031283.1 | MGSIGAASTEFCFDVFRELR |
| | | | | VQHVNENIFYSPFSIISALA |
| | | | | MVYLXARDNTRTQIDKVVHF |
| | | | | DKLPGFGESMEAQCGTSVSV |
| | | | | HSSLRDILTQITKPSDNFSL |
| | | | | SFASRLYAEETYAILPEYLQ |
| | | | | CVKELYKGGLESISFQTAAD |
| | | | | QARELINSWVESQTNGIIKN |
| | | | | ILQPSSVDSQTTMVLVNAIY |
| | | | | FKGMWEKAFKDEDTQAMPFR |
| | | | | MTEQESKPVQMMYQVGSFKV |
| | | | | AMVTSEKMKILELPFASGMM |
| | | | | SMFVLLPDEVSGLEQLESTI |
| | | | | SFEKLTEWTSSTMMEERRMK |
| | | | | VYLPRMKMEEKYNLTSVFMA |
| | | | | LGMTDLFSSSANMSGISSTV |
| | | | | SLKMSEAVHAACVEIFEAGR |
| | | | | DVVGSAEAGMDVTSVSEEFR |
| | | | | ADHPFLFFIKHNPTNSILFF |
| | | | | GRWMSP |
| 827 | Ovalbumin- | Cygnus atratus | XP_035408641.1 | MGSIGAASTEFCFDVFRELK |
| | like | (Black swan) | | VQHVNENIFYSPLSIISALA |
| | | | | MVYLGARDNTRAQIDKVVHF |
| | | | | DKIPGFGESMESQCGTSVSV |
| | | | | HSSLRDILTEITKPSDNFSL |
| | | | | SFASRLYAEETYTILPEYLQ |
| | | | | CVKELYKGGLESISFQTAAD |
| | | | | QARELINSWVESQINGIIKN |
| | | | | ILQPSSVDSQTTMVLVNAIY |
| | | | | FKGMWEKAFKDEDTQTMPFR |
| | | | | MTEQESKPVQMMYQVGSFKV |
| | | | | ATVTSEKVKILELPFASGMM |
| | | | | SMCVLLPDEVSGLEQLETTI |
| | | | | SFEKLTEWTSSTMMEERRMK |
| | | | | VYLPRMKMEEKYNLTSVFMA |
| | | | | LGMTDLFSSSANMSGISSTV |
| | | | | SLKMSEAVHAACVEIFEAGR |
| | | | | DVVGSAEAGMDVTSVSEEFR |
| | | | | ADHPFLFFIKHNPTNSILFF |
| | | | | GRWISP |
| 828 | Ovalbumin- | Anser cygnoides | XP_013056574.1 | MGSIGAASTEFCFDVFRELK |
| | like | domesticus | | VQHVNENIFYSPLSIISALA |
| | | (Domastic goose) | | MVYLGARDNTRTQIDQVVHF |
| | | | | DKIPGFGESMEAQCGTSVSV |
| | | | | HSSLRDILTEITKPSDNFSL |
| | | | | SFASRLYAEETYTILPEYLQ |
| | | | | CVKELYKGGLESISFQTAAD |
| | | | | QARELINSWVESQTNGIIKN |
| | | | | ILQPSSVDSQTTMVLVNAIY |
| | | | | FKGMWEKAFKDEDTQTMPFR |
| | | | | MTEQESKPVQMMYQVGSFKL |
| | | | | ATVTSEKVKILELPFASGMM |
| | | | | SMCVLLPDEVSGLEQLETTI |
| | | | | SFEKLTEWTSSTMMEERRMK |
| | | | | VYLPRMKMEEKYNLTSVFMA |
| | | | | LGMTDLFSSSANMSGISSTV |
| | | | | SLKMSEAVHAACVEIFEAGR |
| | | | | DVVGSAEAGMDVTSVSEEFR |
| | | | | ADHPFLFFIKHNPSNSILFF |
| | | | | GRWISP |

In some embodiments, an egg protein can be a protein that is typically found in an egg without a yolk. Yolkless eggs can comprise wind eggs, dwarf eggs, or fart eggs. Egg proteins may include proteins present in the yolk portion of an egg. In some embodiments, the egg protein is typically found in a fertilized egg.

### Chordates

In some embodiments, a transgene of the disclosure encodes a chordate protein, wherein the chordae is a vertebrate. Illustrative vertebrates are described below.

In some embodiments, a vertebrate is a mammal. For example, in some embodiments, the vertebrate is a bovine. Illustrative bovine species includes, but are not limited to: Holstein, jersey, brown swiss, guernsey, Ayrshire, red and white Holstein, milking shorthorn, simmental, French brown, tux-zillertal, marnau-werdenfel, Icelandic, Danish jersey, aldemey, abigar, Chinese black, agerolese, Australian milking zebu, achham, aulie-ata, Australian Friesian, Jamaica hope, burlina, and butana and kenana. In some cases, the bovine is selected from the group consisting of: Holstein, Jersey, Brown Swiss, Guernsey, Ayrshire, Milking Shorthorn, and Red and White Holstein.

In some embodiments, the vertebrate is a placental mammal. The placental mammals belong to the sub-class Eutheria. In some embodiments, a mammal of the disclosure is a placental mammal selected from the group consisting of a: camel, goat, cow, yak, buffalo, horse, donkey, zebu, sheep, reindeer, giraffe, and cockroach.

In some embodiments, the vertebrate is a bird. Exemplary birds comprise any one of a: chicken, turkey, duck, goose, pheasant, quail, ostrich, guinea fowl, rhea, bantam, pigeon, emu, and dodo, penguin. In some embodiments, the vertebrate is a domesticated bird and/or a bird that are bred to produce eggs for consumption.

In some embodiments, the vertebrate is a chicken. In some embodiments, the vertebrate is a hybrid chicken. Hybrid chickens are bred to lay more eggs than their unmodified or unbred counterparts. In some embodiments, the vertebrate is a chicken selected from a golden comet, Rhode Island red, leghorn, Sussex, Plymouth rock, Ancona, bamevelder, hamburg, maran, buff orpington, easter egger, Ameraucana, Australorp, Delaware, Euskal oiloa, Faverolle, Golden laced Wyandotte, Isa brown, Jaerhon, New Hampshire red, Red sex link, or Welsummer. In some embodiments, the vertebrate is a chicken selected from: australorp, white leghom, Sussex, goldline, hybrid, Plymouth Rock, and Rhode Island Red.

In some embodiments, the vertebrate is a non-bird animal such as a turtle, iguana, alligator, snake, platypus, echidna, reptile, fish, amphibian, insect, lizard, crocodile, alligator, crab, shrimp, ant eater, and modified versions thereof.

In some embodiments, the vertebrate is a marsupial. Marsupials give birth to barely formed offspring, and the baby grows in a pouch on the mother's belly. Marsupial mammals belong to the Sub-class Metatheria.

### Host Cells

Also provided herein are host cells for expressing a transgene of interest. In some embodiments, a protein encoded by a transgene of interest accumulates at a high level in the host cell. In some embodiments, an RNA of interest accumulates at a high level in the host cell. In some embodiments, an RNA of interest has an increased half-life in the host cell.

In some embodiments, the host cell may be a plant cell. For example, the host cell may be a plant cell isolated or derived from any one of the plant species described above. In some embodiments, the host cell can be isolated or derived from a species which is not a plant.

Provided herein are plants, transgenic plants, and portions thereof (for example host cells from plants) that comprise any of the transgene or modifications disclosed herein. Plants may be in any condition including but not limited to dead, alive, pre-germination, post-germination, flowering, seed stage, and combinations thereof. A plant may be edible. A plant may be inedible or poisonous. In some cases, a plant is a crop.

In some embodiments, a plant is a monocot. For example, in some embodiments, the plant may be a monocot selected from turf grass, maize (corn), rice, oat, wheat, barley, sorghum, orchid, iris, lily, onion, palm, and duckweed.

In some embodiments, a plant is a dicot. For example, in some embodiments, the plant may be a dicot selected from Arabidopsis, tobacco, tomato, potato, sweet potato, cassava, alfalfa, lima bean, pea, chickpea, soybean, carrot, strawberry, lettuce, oak, maple, walnut, rose, mint, squash, daisy, Quinoa, buckwheat, mung bean, cow pea, lentil, lupin, peanut, fava bean, French beans (i.e., common beans), mustard, or cactus. In some embodiments, the plant is a soybean (Glycine max). In some embodiments, the plant is Arabidopsis thaliana.

In some embodiments, a plant is a non-vascular plant selected from moss, liverwort, homwort or algae. In some embodiments, the plant is a vascular plant reproducing from spores (e.g., a fern).

Exemplary plants that can be used with the compositions and methods of the disclosure include but are not limited to: spermatophytes (spermatophyta), acrogymnospermae, angiosperms (magnoliophyta), ginkgoidae, pinidae, mesangiospermae, cycads, Ginkgo, conifers, gnetophytes, Ginkgo biloba, cypress, junipers, thuja, cedarwood, pines, angelica, caraway, coriander, cumin, fennel, parsley, dill, dandelion, helichrysum, marigold, mugwort, safflower, camomile, lettuce, wormwood, calendula, citronella, sages, thyme, chia seed, mustard, olive, coffee, capsicum, eggplant, paprika, cranberry, kiwi, vegetable plants (e.g., carrot, celery), tagetes, tansy, tarragon, sunflower, wintergreen, basil, hyssop, lavender, lemon verbena, marjoram, melissa, patchouli, pennyroyal, peppermint, rosemary, sesame, spearmint, primroses, samara, pepper, pimento, potato, sweet potato, tomato, blueberry, nightshades, petunia, morning glory, lilac, jasmin, honeysuckle, snapdragon, psyllium, wormseed, buckwheat, amaranth, chard, quinoa, spinach, rhubarb, jojoba, cypselea, chlorella, manila, hazelnut, canola, kale, bok choy, rutabaga, frankincense, myrrh, elemi, hemp, pumpkin, squash, curcurbit, manioc, dalbergia, legume plants (e.g., alfalfa, lentils, beans, clovers, peas, fava coceira, frijole bola roja, frijole negro, lespedeza, licorice, lupin, mesquite, carob, soybean, peanut, tamarind, wisteria, cassia, chickpea, garbanzo, fenugreek, green pea, yellow pea, snow pea, lima bean, fava bean), geranium, flax, pomegranate, cotton, okra, neem, fig, mulberry, clove, eucalyptus, tea tree, niaouli, fruiting plants (e.g., apple, apricot, peach, plum, pear, nectarine), strawberry, blackberry, raspberry, cherry, prune, rose, tangerine, citrus (e.g., grapefruit, lemon, lime, orange, bitter orange, mandarin), mango, citrus bergamot, buchu, grape, broccoli, brussels, sprout, camelina, cauliflower, rape, rapeseed (canola), turnip, cabbage, cucumber, watermelon, honeydew melon, zucchini, birch, walnut, cassava, baobab, allspice, almond, breadfruit, sandalwood, macadamia, taro, tuberose, aloe vera, garlic, onion, shallot, vanilla, yucca, vetiver, galangal, barley, corn, curcuma aromatica, ginger, lemon grass, oat, palm, pineapple, rice, rye, sorghum, triticale, turmeric, yam, bamboo, barley, cajuput, canna, cardamom, maize, oat, wheat, cinnamon, sassafras, lindera benzoin, bay laurel, avocado, ylang-ylang, mace, nutmeg, moringa, horsetail, oregano, cilantro, chervil, chive, aggregate fruits, grain plants, herbal plants, leafy vegetables, non-grain legume plants, nut plants, succulent plants, land plants, water plants, delbergia, millets, drupes, schizocarps, flowering plants, non-flowering plants, cultured plants, wild plants, trees, shrubs, flowers, grasses, herbaceous plants, brushes, lianas, cacti, green algae, tropical plants, subtropical plants, temperate plants, and derivatives and crosses thereof.

In some embodiments, the host cell comprises a non-plant cell. Exemplary non-plant host cells can be isolated or derived from a microbe, algae, fungi, yeast, and the like. Examples of microbes that may be used as host cells include but are not limited to firmicutes, cyanobacteria (blue-green algae), oscillatoriophcideae, bacillales, lactobacillales, oscillatoriales, bacillaceae, lactobacillaceae, Acetobacter suboxydans, Acetobacter xylinum, Actinoplane missouriensis, Arthrospira platensis, Arthrospira maxima, Bacillus cereus, Bacillus coagulans, Bacillus subtilus, Bacillus cerus, Bacillus licheniformis, Bacillus stearothermophilus, Bacillus subtilis, Escherichia coli, Lactobacillus acidophilus, Lactobacillus bulgaricus, Lactococcus lactis, Lactococcus lactis Lancefield Group N, Lactobacillus reuteri, Leuconostoc citrovorum, Leuconostoc dextranicum, Leuconostoc mesenteroides strain NRRL B-512(F), Micrococcus lysodeikticus, Spirulina, Streptococcus cremoris, Streptococcus lactis, Streptococcus lactis subspecies diacetylactis, Streptococcus thermophilus, Streptomyces chattanoogensis, Streptomyces griseus, Streptomyces natalensis, Streptomyces olivaceus, Streptomyces olivochromogenes, Streptomyces rubiginosus, Tetrahymena thermophile, Tetrahymena hegewischi, Tetrahymena hyperangularis, Tetrahymena malaccensis, Tetrahymena pigmentosa, Tetrahymena pyriformis, and Tetrahymena vorax, and Xanthomonas campestris, and derivatives and crosses thereof.

Examples of algae that may be used as host cells include but are not limited to green algae (e.g., Chlorella), brown algae (e.g., Alaria marginata, Analipus japonicus, Ascophyllum nodosum, Ecklonia sp, Eisenia bicyclis, Hizikia fusiforme, Kjellmaniella gyrata, Laminaria angustata, Laminaria longirruris, Laminaria Longissima, Laminaria ochotensis, Laminaria claustonia, Laminaria saccharina, Laminaria digitata, Laminaria japonica, Macrocystis pyrifera, Petalonia fascia, Scytosiphon lome), red algae (e.g., Gigartinaceae, Soliericeae, Chondrus crispus, Chondrus ocellatus, Eucheuma cottonii, Eucheuma spinosum, Furcellaria fastigiata, Gracilaria bursa-pastoris, Gracilaria lichenoides, Gloiopeltis furcata, Gigartina acicularis, Gigartina bursa-pastoris, Gigartina pistillata, Gigartina radula, Gigartina skottsbergii, Gigartina stellata, Palmaria palmata, Porphyra columbina, Porphyra crispata, Porhyra deutata, Porhyra perforata, Porhyra suborbiculata, Porphyra tenera, Porphyridium cruentum, Porphyridium purpureum, Porphyridium aerugineum, Rhodella maculate, Rhodella reticulata, Rhodella violacea, Rhodophyceae, Rhodymenia palmata), and derivatives and crosses thereof.

Examples of fungi that may be used as host cells include but are not limited to Aspergillus sp., Aspergillus nidulans, Aspergillus niger, Aspergillus niger var. awamori, Aspergillus oryzae, Candida albicans, Candida etchellsii, Candida guilliermondii, Candida humilis, Candida lipolytica, Candida pseudotropicalis, Candida utilis, Candida versatilis, Chrysosporium lucknowense, Debaryomyces hansenii, Endothia parasitica, Eremothecium ashbyii, Fusarium sp., Fusarium gramineum, Fusarium moniliforme, Fusarium venenatum, Hansenula polymorpha, Kluyveromyces sp., Kluyveromyces lactis, Kluyveromyces marxianus, Kluyveromyces marxianus var. lactis, Kluyveromyces thermotolerans, Morteirella vinaceae var. raffinoseutilizer, Mucor miehei, Mucor miehei var. Cooney et Emerson, Mucor pusillus LindtMyceliophthora thermophile, Neurospora crassa, Penicillium roquefortii, Physcomitrella patens, Pichia sp., Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Rhizopus niveus, Rhodotorula sp., Saccharomyces sp., Saccharomyces bayanus, Saccharomyces beticus, Saccharomyces cerevisiae, Saccharomyces chevalieri, Saccharomyces diastaticus, Saccharomyces ellipsoideus, Saccharomyces exiguus, Saccharomyces florentinus, Saccharomyces fragilis, Saccharomyces pastorianus, Saccharomyces pombe, Saccharomyces sake, Saccharomyces uvarum, Sporidiobolus johnsonii, Sporidiobolus salmonicolor, Sporobolomyces roseus, Trichoderma, Trichoderma reesei, Xanthophyllomyces dendrorhous, Yarrowia lipolytica, Zygosaccharomyces rouxii, and derivatives and crosses thereof.

Exemplary yeast that may be used as host cells include but are not limited to: a Kluyveromyces sp., Pichia sp., Saccharomyces sp., Tetrahymena sp., Yarrowia sp., Hansenula sp., Blastobotrys sp., Candida sp., Zygosaccharomyces sp., and Debaryomyces sp. Additional non-limiting examples of yeast strains that can be used as the host cell are Kluyveromyces lactis, Kluyveromyces marxianus, Saccharomyces cerevisiae, and Pichia pastoris. Additional species of yeast strains that can be used as host cells are known in the art.

Also provided herein are non-plant host cells that can be cultivated. The culturing of transgenic host cells can be performed in any fermentation vessel, including but not limited to a culture plate, a flask, or a fermentor (e.g., stirred tank fermentor, an airlift fermentor, a bubble column fermentor, a fixed bed bioreactor, or any combination thereof), and at any scale known in the art. Culture media for use in such fermentations processes may include any culture medium in which the recombinant host cells provided herein can grow and/or remain viable. In some embodiments, the culture media are aqueous media comprising carbon, nitrogen (e.g., anhydrous ammonia, ammonium sulfate, ammonium nitrate, diammonium phosphate, monoammonium phosphate, ammonium polyphosphate, sodium nitrate, urea, peptone, protein hydrolysates, yeast extract), and phosphate sources. The culture media can further comprise salts, minerals, metals, other nutrients, emulsifying oils, and surfactants. Non-limiting examples of carbon sources include monosaccharides, disaccharides, polysaccharides, acetate, ethanol, methanol, methane, or one or more combinations thereof. Non-limiting examples of monosaccharides include dextrose (glucose), fructose, galactose, xylose, arabinose, and combinations thereof. Non-limiting examples of disaccharides include sucrose, lactose, maltose, trehalose, cellobiose, and combinations thereof. Non-limiting examples of polysaccharides include starch, glycogen, cellulose, amylose, hemicellulose, and combinations thereof. Conditions for production of the recombinant proteins are those under which the recombinant host cells provided herein can grow and/or remain viable. Non-limiting examples of such conditions include suitable pH, suitable temperature, and suitable oxygenation. In some embodiments, the culture media further comprise proteases (e.g., plant-based proteases) that can prevent degradation of the recombinant proteins, protease inhibitors that reduce the activity of proteases that can degrade the recombinant proteins, and/or sacrificial proteins that siphon away protease activity.

### Transgenic Organisms, Including Plants and Host Cells

Also provided herein are transgenic organisms, such as plants, comprising or expressing one or more chordate proteins of the disclosure. In some embodiments, the transgenic host cells comprise an exogenous RNA sequence that encodes a chordate protein selected from ovalbumin, β-Lactoglobulin, or combinations thereof.

In some embodiments, the transgenic plants stably express the chordate protein. In some embodiments, the transgenic plants transiently express the chordate protein. In some embodiments, the transgenic plants and/or host cell stably express the chordate protein in the plant or cell thereof in an amount of at least 1% per the total protein weight of the soluble protein extractable from the plant or cell thereof. For example, the transgenic plants and/or host cell may stably express the chordate protein in an amount of at least 1%, at least 1.5%, at least 2%, at least 2.5%, at least 3%, at least 3.5%, at least 4%, at least 4.5%, at least 5%, at least 5.5%, at least 6%, at least 6.5%, at least 7%, at least 7.5%, at least 8%, at least 8.5%, at least 9%, at least 9.5%, at least 10%, at least 10.5%, at least 11%, at least 11.5%, at least 12%, at least 12.5%, at least 13%, at least 13.5%, at least 14%, at least 14.5%, at least 15%, at least 15.5%, at least 16%, at least 16.5%, at least 17%, at least 17.5%, at least 18%, at least 18.5%, at least 19%, at least 19.5%, at least 20%, including all ranges and subranges therebetween, or more of total protein weight of soluble protein extractable from the plant and/or host cell.

In some embodiments, the transgenic plants and/or host cell thereof may stably express the chordate protein in an amount of less than about 1% of the total protein weight of soluble protein extractable from the plant or cell thereof. In some embodiments, the transgenic plants or cell thereof stably express the chordate protein in the range of about 1% to about 2%, about 3% to about 4%, about 4% to about 5%, about 5% to about 6%, about 6% to about 7%, about 7% to about 8%, about 8% to about 9%, about 9% to about 10%, about 10% to about 11%, about 11% to about 12%, about 12% to about 13%, about 13% to about 14%, about 14% to about 15%, about 15% to about 16%, about 16% to about 17%, about 17%, to about 18%, about 18% to about 19%, about 19% to about 20%, or more than about 20%, including all ranges and subranges therebetween, of the total protein weight of soluble protein extractable from the plant and/or host cell thereof.

In some embodiments, the transgenic plant or host cell stably expresses the chordate protein in an amount in the range of about 0.5% to about 3%, about 1% to about 4%, about 1% to about 5%, about 2% to about 5%, about 1% to about 10%, about 2% to about 10%, about 3% to about 10%, about 5 to about 12%, about 4% to about 10%, or about 5% to about 10%, about 4% to about 8%, about 5% to about 15%, about 5% to about 18%, about 10% to about 20%, or about 1% to about 20%, including all ranges and subranges therebetween, of the total protein weight of soluble protein extractable from the plant and/or host cell thereof. In some embodiments, the chordate protein is ovalbumin or β-Lactoglobulin expressed from about 1% to 3% of the total protein weight of soluble protein extractable from the plant and/or host cell thereof.

In some embodiments, the chordate protein is expressed at a level at least 2-fold higher than a protein expressed without a method comprising RNA stabilization in a plant or host cell thereof. For example, in some embodiments, the chordate protein is expressed at a level at least 2-fold, at least 2.5-fold, at least 3-fold, at least 3.5-fold, at least 4-fold, at least 4.5-fold, at least 5-fold, at least 5.5-fold, at least 6-fold, at least 7-fold, at least 7.5-fold, at least 8-fold, at least 8.5-fold, at least 9-fold, at least 9.5-fold, at least 10-fold, at least 25-fold, at least 50-fold, or at least 100-fold higher, including all ranges and subranges therebetween, than a protein expressed without RNA stabilization in a plant and/or host cell thereof.

In some embodiments, the chordate protein allows for accumulation of a chordate protein in a host cell at least 2-fold higher than a casein protein expressed without RNA stabilization in a plant or host cell. For example, in some embodiments, a chordate protein accumulates in a host cell and/or plant at least 2-fold, at least 2.5-fold, at least 3-fold, at least 3.5-fold, at least 4-fold, at least 4.5-fold, at least 5-fold, at least 5.5-fold, at least 6-fold, at least 7-fold, at least 7.5-fold, at least 8-fold, at least 8.5-fold, at least 9-fold, at least 9.5-fold, at least 10-fold, at least 25-fold, at least 50-fold, or at least 100-fold higher, including all ranges and subranges therebetween, than a chordate protein expressed without any of the RNA stabilization methods provided herein.

In some embodiments, the chordate protein is stably expressed in the plant and/or host cell in an amount of 1% or higher per the total protein weight of the soluble protein extractable from the plant and/or host cell. In some embodiments, the chordate protein is stably expressed in the plant and/or host cell in an amount of 2% or higher per the total protein weight of the soluble protein extractable from the plant and/or host cell. In some embodiments, the chordate protein is stably expressed in the plant and/or host cell in an amount of 3% or higher per the total protein weight of the soluble protein extractable from the plant and/or host cell. In some embodiments, the chordate protein is stably expressed in the plant and/or host cell in an amount of 4% or higher per the total protein weight of the soluble protein extractable from the plant and/or host cell. In some embodiments, the chordate protein is stably expressed in the plant and/or host cell in an amount of 5% or higher per the total protein weight of the soluble protein extractable from the plant and/or host cell. In some embodiments, the chordate protein is stably expressed in the plant and/or host cell in an amount of 6% or higher per the total protein weight of the soluble protein extractable from the plant and/or host cell. In some embodiments, the chordate protein is stably expressed in the plant and/or host cell in an amount of 7% or higher per the total protein weight of the soluble protein extractable from the plant and/or host cell. In some embodiments, the chordate protein is stably expressed in the plant and/or host cell in an amount of 8% or higher per the total protein weight of the soluble protein extractable from the plant and/or host cell. In some embodiments, the chordate protein is stably expressed in the plant and/or host cell in an amount of 9% or higher per the total protein weight of the soluble protein extractable from the plant and/or host cell. In some embodiments, the chordate protein is stably expressed in the plant and/or host cell in an amount of 10% or higher per the total protein weight of the soluble protein extractable from the plant and/or host cell. In some embodiments, the chordate protein is stably expressed in the plant and/or host cell in an amount of 110% or higher per the total protein weight of the soluble protein extractable from the plant and/or host cell. In some embodiments, the chordate protein is stably expressed in the plant and/or host cell in an amount of 12% or higher per the total protein weight of the soluble protein extractable from the plant and/or host cell. In some embodiments, the chordate protein is stably expressed in the plant and/or host cell in an amount of 13% or higher per the total protein weight of the soluble protein extractable from the plant and/or host cell. In some embodiments, the chordate protein is stably expressed in the plant and/or host cell in an amount of 14% or higher per the total protein weight of the soluble protein extractable from the plant and/or host cell. In some embodiments, the chordate protein is stably expressed in the plant and/or host cell in an amount of 15% or higher per the total protein weight of the soluble protein extractable from the plant and/or host cell. In some embodiments, the chordate protein is stably expressed in the plant and/or host cell in an amount of 16% or higher per the total protein weight of the soluble protein extractable from the plant and/or host cell. In some embodiments, the chordate protein is stably expressed in the plant and/or host cell in an amount of 17% or higher per the total protein weight of the soluble protein extractable from the plant and/or host cell. In some embodiments, the chordate protein is stably expressed in the plant and/or host cell in an amount of 18% or higher per the total protein weight of the soluble protein extractable from the plant and/or host cell. In some embodiments, the chordate protein is stably expressed in the plant and/or host cell in an amount of 19% or higher per the total protein weight of the soluble protein extractable from the plant and/or host cell. In some embodiments, the chordate protein is stably expressed in the plant and/or host cell in an amount of 20% or higher per the total protein weight of the soluble protein extractable from the plant and/or host cell.

In some embodiments, a transformed plant and/or host cell comprises in its genome: a recombinant DNA construct encoding a chordate protein, wherein the chordate protein comprises lysozyme, ovalbumin, ovotransferrin, or ovoglobulin. In some embodiments, the chordate protein is ovalbumin. A subject DNA construct encoding ovalbumin can comprise any of the aforementioned stabilization elements and/or modifications within any of the elements including but not limited to a promoter, terminator, codon optimization, KDEL, intron, ubiquitin monomer, 5′UTR, 3′UTR, and combinations thereof. In some embodiments, a DNA construct encoding ovalbumin comprises a promoter selected from the group consisting of: BnNap, gmSeed2, gmSeed12, pvPhas, and combinations thereof. In some embodiments, a DNA construct encoding ovalbumin comprises a signal peptide selected from the group consisting of: sig11, sig 2, coixss, sig12, and combinations thereof. In some embodiments, no signal peptide is comprised within the DNA construct. In some embodiments, a DNA construct encoding ovalbumin comprises a terminator sequence selected from the group consisting of arcT, Rb7T, EUT, and combinations thereof. In some embodiments, a double terminator is used. A double terminator can be EUT:Rb7T. In some embodiments, a DNA construct encoding ovalbumin comprises a KDEL sequence. In some embodiments, a DNA construct encoding ovalbumin comprises an exogenous or ectopically located intron sequence. In some embodiments, a DNA construct encoding ovalbumin comprises an exogenous or ectopically located glnB1 sequence.

In some embodiments, a transformed plant and/or host cell comprises in its genome a recombinant DNA construct encoding a β-Lactoglobulin protein. A subject DNA construct encoding β-Lactoglobulin can comprise any of the aforementioned stabilization elements and/or modifications within any of the elements including but not limited to a promoter, terminator, codon optimization, KDEL, intron, ubiquitin monomer, 5′UTR, 3′UTR, and combinations thereof. In some embodiments, a DNA construct encoding β-Lactoglobulin comprises a promoter selected from the group consisting of: BnNap, gmSeed2, gmSeed12, pvPhas, and combinations thereof. In some embodiments, a DNA construct encoding β-Lactoglobulin comprises a signal peptide selected from the group consisting of: sig11, sig 2, coixss, sig12, and combinations thereof. In some embodiments, a signal peptide is selected from a sequence in Table 4 or Table 11. In some embodiments, a signal peptide comprises a sequence having about 70%, about 75%, about 80%, about 85%, about 90%, about 95%, about 96%, about 97%, about 98%, about 99%, or 100%, including all ranges and subranges therebetween, identity from a sequence in Table 4 or Table 11. In some embodiments, no signal peptide is comprised within the DNA construct. In some embodiments, a DNA construct encoding β-Lactoglobulin comprises a terminator sequence selected from the group consisting of arcT, Rb7T, EUT, and combinations thereof. In some embodiments, a double terminator is used. A double terminator can be EUT: Rb7T. In some embodiments, a DNA construct encoding β-Lactoglobulin comprises a KDEL sequence. In some embodiments, a DNA construct encoding β-Lactoglobulin comprises an exogenous or ectopically located intron sequence. In some embodiments, a DNA construct encoding β-Lactoglobulin comprises an exogenous or ectopically located glnB1 sequence. In some embodiments, the milk protein is β-lactoglobulin and comprises the sequence of SEQ ID NO: 10, or a sequence at least 90% identical thereto.

In some embodiments, constructs encoding ovalbumin or β-Lactoglobulin can comprise a sequence provided in Table 11, Table 12 and/or Table 15 or a sequence having from about: 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, including all ranges and subranges therebetween, relative thereto.

In some embodiments, a transformed plant and/or host cell comprises in its genome a recombinant DNA construct encoding a milk protein. In some embodiments, the milk protein is α-lactalbumin, lysozyme, lactoferrin, lactoperoxidase, or an immunoglobulin (e.g., IgA, IgG, IgM, or IgE).

In some embodiments, a transformed plant and/or host cell comprises in its genome: a recombinant DNA construct encoding a casein protein. In some embodiments, a transformed plant and/or host cell comprises in its genome: a recombinant DNA construct encoding a casein protein selected from α-S1 casein, α-S2 casein, β-casein, and κ-casein. In some embodiments, the milk protein is α-S1 casein. In some embodiments, the milk protein is α-S1 casein and comprises the sequence SEQ ID NO: 8, or a sequence at least 90% identical thereto. In some embodiments, the milk protein is α-S2 casein. In some embodiments, the milk protein is α-S2 casein and comprises the sequence SEQ ID NO: 84, or a sequence at least 90% identical thereto. In some embodiments, the milk protein is β-casein. In some embodiments, the milk protein is β-casein and comprises the sequence of SEQ ID NO: 6, or a sequence at least 90% identical thereto. In some embodiments, the casein protein is κ-casein. In some embodiments, the casein protein is κ-casein and comprises the sequence of SEQ ID NO: 4, or a sequence at least 90% identical thereto. In some embodiments, the casein protein is para-κ-casein. In some embodiments, the casein protein is para-κ-casein and comprises the sequence of SEQ ID NO: 2, or a sequence at least 90% identical thereto.

In some embodiments, a transformed plant and/or host cell comprises in its genome: a recombinant DNA construct encoding hemoglobin, collagen, IgM, or IgE.

The transgenic plants and/or host cells described herein may be generated by various methods known in the art. For example, a DNA construct encoding a chordate protein may be contacted with a plant, or a portion thereof, and the plant may then be maintained under conditions wherein the chordate protein is expressed. In some embodiments, the DNA construct is introduced into the plant, or part thereof, using one or more methods for plant transformation known in the art, such as Agrobacterium-mediated transformation, particle bombardment-medicated transformation, electroporation, and microinjection.

In some embodiments, a method for expressing a chordate protein in a plant cell comprises: (a) contacting a plant cell with a DNA construct, thereby generating a transformed plant cell; and (b) cultivating a plant that comprises the transformed plant cell, thereby generating a transformed plant. In some embodiments, the chordate protein is expressed in the amount of at least about 1%, at least 2%, at least 3%, at least 4%, or at least 5% or higher per total protein weight of soluble protein extractable from the transformed plant cell. In some embodiments, the chordate protein is expressed in the amount of at least about 1%-3%, 3%-5%, 1-5%, 2-5%, 5-10%, including all ranges and subranges therebetween, or higher per total protein weight of soluble protein extractable from the transformed plant cell. In embodiments, a method can further comprise isolating a portion of the transformed plant including but not limited to any plant tissue: leaf, stem, root, tuber, seed, branch, pubescence, nodule, leaf axil, flower, pollen, stamen, pistil, petal, peduncle, stalk, stigma, style, bract, fruit, trunk, carpel, sepal, anther, ovule, pedicel, needle, cone, rhizome, stolon, shoot, seed, pericarp, endosperm, placenta, berry, stamen, and/or leaf sheath. In some cases, a method comprises isolating a seed from a transformed plant.

In some cases, a transgenic plant and/or host cell comprises a level of a chordate protein encoded by any of the DNA constructs provided herein that can be measured via an in vitro assay. A person of skill in the art will readily identify suitable in vitro assays to measure protein levels including but not limited to: ELISA, western blot, protein quantitation ratioing, mass spectrometry, and the like. In embodiments, a level of a chordate protein encoded by a provided transgene construct is increased by at least about: 0.5 fold, 1-fold, 2-fold, 3-fold, 4-fold, 5-fold, 8-fold, 10-fold, 15-fold, 20-fold, 30-fold, 40-fold, 50-fold, 70-fold, 100-fold, 120-fold, 150-fold, 175-fold, 200-fold, 300-fold, 400-fold, or up to about 500-fold as compared to an otherwise comparable host cell or transgenic plant lacking modification with any of the disclosed transgenes and/or RNA stabilization methods.

Methods of Processing Chordate Proteins from Host Cells

The chordate protein may be extracted from a host cell, such as from a plant, using standard methods known in the art. Any chordate protein can be expressed in a host cell and/or transgenic plant. In some embodiments, the chordate protein is ovalbumin and/or β-Lactoglobulin expressed in a soybean plant.

In some embodiments, the protein may be extracted using solvent or aqueous extraction. In some embodiments, the oil may be separated from the protein using hexane or ethanol extraction to produce a white flake. The protein may be extracted from the white flake using controlled temperature in an aqueous buffered environment (e.g., carbonate, citrate), in order to control the pH. The chordate protein can be separated from the host cell proteins using selective precipitation of one or more of the proteins with centrifugation or filtration methods. In some embodiments, one or more additives may be used to aid the extraction processes (e.g., salts, protease/peptidase inhibitors, osmolytes, solvents, reducing agents, etc.) The following step is processing the chordate protein into a food product. In some embodiments, only one protein from a chordate is used in a product. In some embodiments, more than one chordate protein is used in a product. In some embodiments, all chordate proteins may be used in a product. In some embodiments, a chordate protein may be used itself in a food product. The product is then formulated as desired.

In some embodiments a method comprises collecting seeds from a host cell plant. After seeds are collected, hulled and/or ground, and chordate protein has been extracted, the chordate protein is separated from other seed protein. In some embodiments, this separation is not 100% efficient, meaning that the “other seed protein” fraction may still contain some residual host cell protein. For example, in some embodiments, the other seed protein fraction may comprise about 0.1%, about 0.3%, about 0.5%, about 0.7%, about 1%, about 2%, about 3%, about 4%, about 5%, about 6%, about 7%, about 8%, about 9%, about 20%, about 20%, about 30%, or about 50%, including all ranges and subranges therebetween, chordate protein by weight. The other seed protein fraction may then be used directly in a food composition. Alternatively, the other seed protein fraction may be combined with concentrated chordate protein. In some embodiments, the other seed protein fraction is combined with one or more of the constituent proteins from the chordate protein. In some embodiments, the other seed protein fraction is combined with all of the constituent proteins from the chordate protein.

It may be advantageous to use a seed processing composition comprising plant protein and a chordate protein (e.g., about 0.1%, about 0.3%, about 0.5%, about 0.7%, about 1%, about 2%, about 3%, about 4%, about 5%, about 6%, about 7%, about 8%, about 9%, about 20%, about 20%, about 30%, or about 50%, including all ranges and subranges therebetween, chordate protein by weight) as an ingredient in a food composition. Using both (i) a chordate protein produced by a seed and (ii) other protein extracted from the seed allows for efficient use of resources and reduces waste. Such processes may simplify food manufacturing processes and reduce the unit cost to manufacture each product. Thus, provided herein is a method of making a food composition, the method comprising: (i) expressing a chordate protein in a transformed plant; and (ii) preparing a food composition comprising the chordate protein and plant protein from the same transformed plant in which the chordate protein was produced. In some embodiments, the transformed plant is a soybean. In some embodiments, the transformed plant is pea.

### Food Compositions

Any of the compositions and methods provided herein can be used to generate a food composition. In some embodiments, host cells of the disclosure are modified to comprise and/or express transgene sequences that encode for a chordate protein. In some embodiments, a host cell is a mammalian host cell. In some embodiments, a host cell is a plant cell. Any one of a mammalian or plant cell can be modified to comprise or express a chordate protein. In some embodiments, a plant expresses a chordate protein selected from ovalbumin and β-Lactoglobulin.

In some embodiments, a plant protein composition comprising a chordate protein is used to produce a food composition. The food composition may be, for example, a meat analog, a nutritional bar, a bakery product, a beverage, mashed potatoes, or candy. In some embodiments, the food composition is for a human. In some embodiments, the food composition is for a companion animal (e.g., a dog, cat, rabbit, hamster, guinea pig, horse, etc.) For example, the food composition may be pet food. In some embodiments, the food composition is for a pediatric human.

Also provided herein are various compositions prepared during a method of making a food composition. For example, in some embodiments, a seed processing composition is provided comprising ovalbumin and/or β-Lactoglobulin. In some embodiments, a seed processing composition comprises (a) a chordate protein comprising i) a full-length ovalbumin component; and ii) a β-lactoglobulin component; and (b) plant seed tissue. In some embodiments, a seed processing composition comprises (a) a chordate protein comprising i) an ovalbumin component; and ii) a β-lactoglobulin component; and (b) plant seed tissue. In some embodiments, a seed processing composition comprises (a) a chordate protein comprising i) an egg or milk protein (e.g., an ovalbumin or β-Lactoglobulin protein); and ii) a second protein (i.e., a fusion partner); and (b) plant seed tissue. In some embodiments, the plant seed tissue is ground. In some embodiments, the plant seed tissue is from soybean. In some embodiments, the seed processing composition comprises at least one member selected from the group consisting of: enzyme (e.g., chymosin), protease, extractant, solvent, buffer, additive, salt, protease inhibitor, peptidase inhibitor, osmolyte, and reducing agent.

In some embodiments, a protein concentrate composition is provided. In some embodiments, the protein concentrate composition comprises: a chordate protein, comprising i) a full-length ovalbumin component; and/or ii) a β-lactoglobulin component. In some embodiments, the protein concentrate composition comprises: a chordate protein, comprising i) an ovalbumin component; and/or ii) a β-lactoglobulin component. In some embodiments, the protein concentrate composition comprises: a chordate protein, comprising i) an egg or milk protein (e.g., ovalbumin or β-Lactoglobulin protein); and ii) a second protein. In some embodiments, the chordate protein is present in an enriched amount, relative to other components present in the composition. In some embodiments, there is substantially no plant seed tissue present in the protein concentrate composition. In some embodiments, the protein concentrate composition further comprises at least one member selected from the group consisting of: enzyme (e.g., chymosin), protease, extractant, solvent (e.g., ethanol, hexane, phenol), buffer, additive, salt, protease inhibitor, peptidase inhibitor, osmolyte, and reducing agent.

In some embodiments, the food composition is a solid. In some embodiments, the food composition is a liquid. In some embodiments, the food composition is a powder.

In some embodiments, the food composition is a solid phase, protein-stabilized emulsion. In some embodiments, the food composition is a colloidal suspension.

In some embodiments, the chordate proteins and transgenic plants described herein may be used to prepare a food composition such as a meat composition. A meat composition of the disclosure can comprise a milk protein (e.g., β-lactoglobulin protein). In some embodiments, meat compositions of the disclosure can comprise β-lactoglobulin isolated from a plant, for example a soybean plant. In some embodiments, meat compositions of the disclosure can comprise β-lactoglobulin isolated from a plant, for example a soybean plant, and a combination of methylcellulose and a casein protein of the disclosure. In some embodiments, a meat composition comprises reduced methylcellulose as compared to an otherwise comparable meat composition lacking a casein protein comprised within the meat composition. Various meat compositions are contemplated including, but not limited to: burger, patty, sausage, hot dog, nugget, finger, salad, bouillon powder, bouillon cube, flavor packet, meat ball, meatloaf, and the like.

In some embodiments, the chordate proteins and transgenic plants described herein may be used to prepare a food composition such as one or more egg substitute compositions. An egg substitute composition of the disclosure can comprise an egg protein (e.g., ovalbumin). In some embodiments, egg substitute compositions of the disclosure can comprise ovalbumin isolated from a plant, for example a soybean plant. Various egg substitutes are contemplated including but not limited to: an egg-based sauce (e.g. mayonnaise), dressing or custard; a scramble, omelet, or quiche; or an egg white composition.

In some embodiments, the chordate proteins and transgenic plants described herein may be used to prepare a food composition such as one or more baked goods. A baked good composition of the disclosure can comprise an egg protein (e.g., ovalbumin). In some embodiments, baked goods of the disclosure can comprise ovalbumin isolated from a plant, for example a soybean plant. Various baked goods are contemplated including but not limited to: bars, breads (bagels, buns, rolls, biscuits and loaf breads), cookies, desserts (brownies, cakes, cheesecakes and pies), muffins, pizza, snack cakes, sweet goods (doughnuts, Danish, sweet rolls, cinnamon rolls and coffee cake) and tortillas.

In some embodiments, the chordate proteins and transgenic plants described herein may be used to prepare a food composition such as cheese or processed cheese products. In some embodiments, the food composition is an alternative dairy composition selected such as milk, cream, or butter. The alternative milk composition may be used to prepare alternative dairy compositions such as yogurt and fermented dairy products, directly acidified counterparts of fermented dairy products, cottage cheese, dressing, curds, creme fraiche, toppings, icings, fillings, low-fat spreads, dairy-based dry mixes, frozen dairy products, frozen desserts, desserts, baked goods, soups, sauces, salad dressing, geriatric nutrition, creams and creamers, analog dairy products, follow-up formula, baby formula, infant formula, milk, dairy beverages, acid dairy drinks, smoothies, milk tea, butter, margarine, butter alternatives, growing up milks, low-lactose products and beverages, medical and clinical nutrition products, protein/nutrition bar applications, sports beverages, confections, meat products, analog meat products, meal replacement beverages, and weight management food and beverages.

In some embodiments, the chordate proteins and transgenic plants described herein may be used to prepare a food composition such as tofu or processed tofu products.

In some embodiments the chordate proteins and transgenic plants described herein may be used to prepare a dairy product. In some embodiments, the dairy product is a fermented dairy product. An illustrative list of fermented dairy products includes cultured buttermilk, sour cream, yogurt, skyr, leben, lassi, or kefir. In some embodiments the chordate proteins and transgenic plants described herein may be used to prepare cheese products.

In some embodiments the chordate proteins and transgenic plants described herein may be used to prepare a powder containing a milk protein. In some embodiments, the chordate proteins and transgenic plants described herein may be used to prepare a low-lactose product.

In some embodiments, a method for making a food composition comprises, expressing a recombinant chordate protein of the disclosure in a plant, extracting the recombinant chordate protein from the plant, optionally separating the ovalbumin and/or β-Lactoglobulin from the mammalian or plant protein, and creating a food composition using the chordate protein and/or the milk protein.

In some embodiments, a method of expressing, extracting, and making a food composition from a chordate protein, comprises: expressing a chordate protein in a host cell, the chordate protein comprising a first protein and a second protein; extracting the chordate protein from the host cell; and processing the chordate protein into a food composition. The food composition may be, for example, cheese, processed cheese product, yogurt, fermented dairy product, directly acidified counterpart of fermented dairy product, cottage cheese dressing, frozen dairy product, frozen dessert, dessert, baked good, topping, icing, filling, low-fat spread, dairy-based dry mix, soup, sauce, salad dressing, geriatric nutrition, cream, creamer, analog dairy product, follow-up formula, baby formula, infant formula, milk, dairy beverage, acid dairy drink, smoothie, milk tea, butter, margarine, butter alternative, growing up milk, low-lactose product, low-lactose beverage, medical and clinical nutrition product, protein bar, nutrition bar, sport beverage, confection, meat product, analog meat product, meal replacement beverage, weight management food and beverage, dairy product, cultured buttermilk, sour cream, yogurt, skyr, leben, lassi, kefir, powder containing a milk protein, and low-lactose product. In some embodiments, the food composition is a dairy product. In some embodiments, the food composition is a cheese.

In some embodiments, a method for making a food composition comprises, expressing a recombinant chordate protein of the disclosure in a plant, extracting one or both of the proteins, and creating a food composition using the chordate protein. In some embodiments, the first protein and the second protein are separated from one another in the plant cell, prior to extraction. In some embodiments, the first protein is separated from the second protein after extraction, for example by contacting the chordate protein with an enzyme that cleaves the chordate protein. The enzyme may be, for example, chymosin. In some embodiments, the chordate protein is cleaved using rennet.

Provided herein are also nutraceuticals generated using any of the compositions or methods provided herein. Nutraceuticals are products derived from food sources that can provide extra health benefits, in addition to the basic nutritional value found in foods. Nutraceutical products may prevent chronic diseases, improve health, delay the aging process, increase life expectancy, and/or support the structure or function of the body. In some embodiments, a nutraceutical comprises any one of a: drug, dietary supplement, herbal supplement, food ingredient, antioxidants, fortified dairy products, citrus fruits, vitamins, minerals, herbals, milk, and/or cereals.

### Kits, Containers, and the Like

Provided are also containers, kits, encasements, and the like that comprise any of the compositions provided herein. In some embodiments, a kit is provided for stabilizing RNA in host cells. A kit can also comprise any of the DNA constructs provided, sequences, transgenic cells, transgenic plants, and/or any of the in bulk. For instance, a bushel of a transgenic plant can be provided in contained form. In some embodiments, a DNA construct comprises GmSeed2:sig2:OOVAL2 (intron 1):KDEL:EUT:Rb7T. In some embodiments, a container, kit, and the like comprise a nucleic acid encoding any one of SEQ ID NO: 685, 687, and 695. In some embodiments, a container, kit, and the like comprise a nucleic acid encoding a sequence comprising at least about 85%, 90%, 95%, 96%, 97%, 98%, 99%, or 100% identity to any one of SEQ ID NO: 685, 687, and 695. Transgenic plants manufactured using any of the aforementioned nucleic acids can also be provided in any of the kits, containers, and the like of the disclosure.

Any of the containment forms provided can also comprise additional components such as media, water, soil, plant supplements, and the like to generate and/or cultivate transgenic host cells or transgenic plants.

Some examples of the kits further include instructions for making any of the compositions described herein.

### EXAMPLES

The following experiments demonstrate different strategies employing engineered constructs to increase RNA stability and protein expression, as well as methods of generating and testing the same. While the examples below describe expression in plants, it will be understood by those skilled in the art that the constructs and methods disclosed herein may be tailored for expression in any host organism.

### Example 1: Strategies to Increase RNA Stability and Protein Generation and Study Methodology

### Outline of Exemplary Strategies

Various strategies were tested in order to either increase RNA levels or improve RNA stability, which in turn lead to an increased protein accumulation in soybean seeds. These strategies are listed in Table 9.

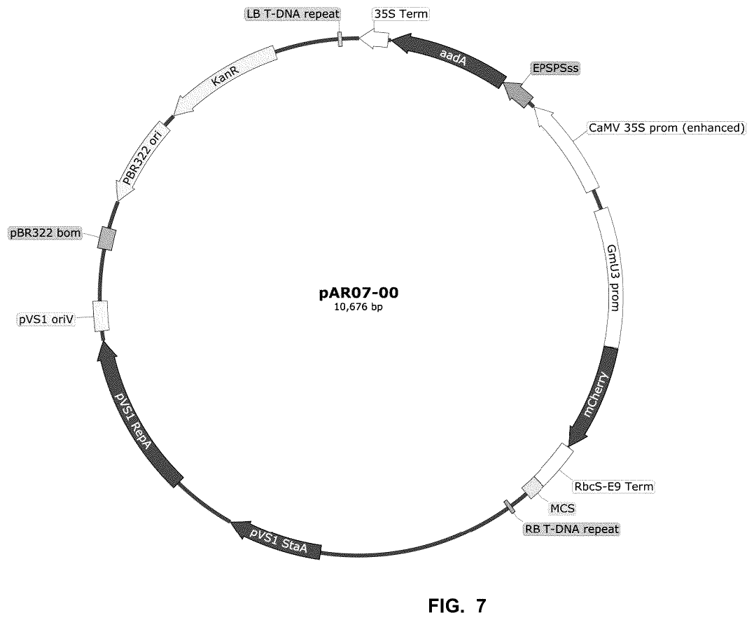

TABLE 9
Summary of strategies that were tested to increase RNA stability
and recombinant protein accumulation in soybean seeds.

| Category | High level strategy | Details |
|---|---|---|
| Transcriptional regulation | Promoter/Terminator | Tested different combinations of promoter and |
| | | terminator. |
| Transcriptional/Translational | Codon optimization | Different transgenes were designed and inserted |
| Regulation/RNA stability | | into the plasmid of FIG. 7 with different codon |
| | | optimization versions. |
| RNA stability | +/− KDEL | Constructs were designed that either contained |
| | | (+) or did not contain (−) a KDEL sequence. |
| | | KDEL acts on the stability of the RNA, and can |
| | | have positive effects on the expression of some |
| | | transgenes. |
| Transcriptional regulation | Intron | Constructs containing one or more exogenous |
| | | introns were designed. Splicing can have a |
| | | positive effect on RNA stability. It was |
| | | hypothesized that by reintroducing introns in |
| | | specific locations of the transgene, RNA levels |
| | | can be enhanced. |
| Transcriptional regulation | 5′ UTR/3′ UTR | The 5′ or 3′ untranslated region (UTR) may act |
| | | on the stability of the mRNA and translation |
| | | efficiency, and may also play a role in |
| | | regulating the transport of mRNAs out of the |
| | | nucleus. Accordingly, constructs were designed |
| | | that either contain or do not contain a 5′ and/or |
| | | a 3′ UTR. |
| Translational regulation | Monomer | Ubiquitin monomers from certain plant species |
| | | were transcriptionally fused to a transgene in |
| | | order to enhance protein expression. The |
| | | ubiquitin monomer is separated from the |
| | | transgene either immediately after or during |
| | | translation, improving translational regulation. |

### Example 2: Use of Promoter and Terminators to Enhance Stability of RNA and Increase Protein Levels

### Study Design and Methodology

Protein accumulation may be increased by modulating levels of gene expression. Since promoter selection is an important factor in determining level of gene expression, various promoters were tested to determine which is able to drive optimal transcription of RNA encoding a desired protein. Specifically, to express OVAL and LG in soybean seeds, several seed specific promoters (BnNap, GmSeed2, GmSeed12 and PvPhas) were tested. Except for PvPhas, most of the promoters were used in combination with the nopaline synthase terminator (nosT) to control for the effect of that element in protein expression.

The first strategy tested use of seed-specific promoters to modulate expression of OVAL or LG in soybean seeds. Various seed-specific promoters BnNap, GmSeed2, GmSeed12 and PvPhas were implemented. Except for PvPhas, most of the promoters were accompanied with the nopaline synthase terminator (nosT) to control for the effect of that element in protein expression.

qPCR analysis was used to determine RNA levels, and enzyme-linked immunosorbent assays (ELISA) or western blots were used to quantify protein expression. qPCR and ELISA data was extracted for all the plasmid constructs that contained OVAL or LG as the transgene.

RNA and protein quantification data was analyzed as follows: (1) ELISA protein quantifications of ovalbumin and β-Lactoglobulin are summarized in Table 10 and Table 12 except for AR07-22 and AR07-23 where the seed samples were only analyzed using western blot; (2) Seed samples for each construct were separated into 3 categories based on their protein expression levels: WT seeds that have below detection threshold expressions; Low expression seeds that have above detection threshold but below 1% TSP expressions; or High expression seeds that have above 1% TSP expressions. ELISA detection thresholds for ovalbumin and 0-Lactoglobulin are 0.023% TSP and 0.063% TSP respectively. The numbers of seeds in the three categories for each construct are summarized in Table 10 and Table 12.

### Results

As described below, Table 10 provides data summarizing relative expression of the ovalbumin (i.e., RNA levels), which is the transcript level of the ovalbumin transgene relative to the native Glycinin 1 gene of all the seeds that were analyzed per construct design (n=number of seeds). Analyzed seeds were collected around 90 days after plants were transferred to soil. Table 10 also shows protein levels (i.e., % Total Soluble protein (% TSP)) of the ovalbumin transgene in all the seeds that were analyzed per construct design (n=number of seeds). Analyzed seeds were collected between 90-120 days after plants were transferred to soil.

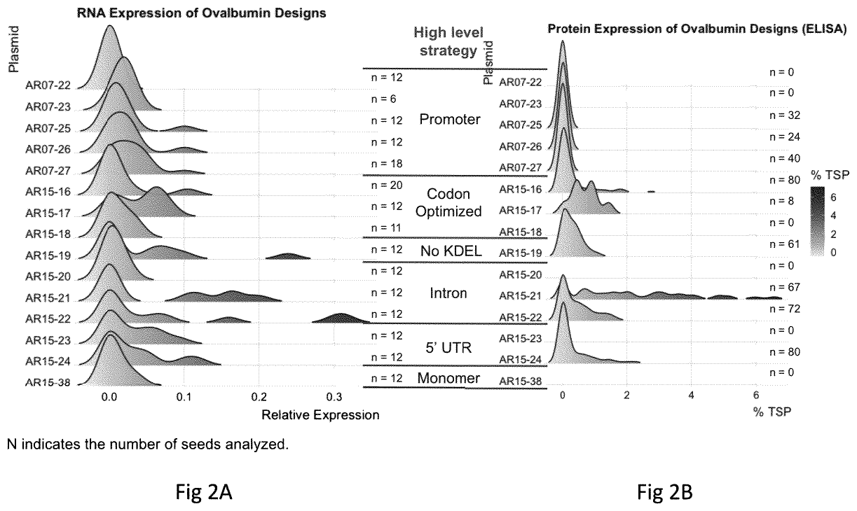

Results show that OVAL expressed under different seed specific promoters (AR07-22, -23, -25, -26 and -27) accumulated at low RNA levels (FIG. 2A), leading to low protein accumulation (FIG. 2B). GmSeed12:sig12 (AR07-27) and PvPhas (AR07-27) were the two designs that had the highest RNA and protein level (Table 10, plasmid ID AR07-26). The highest RNA and protein level recorded was 0.11 times the level of glycinin1 and 0.08% TSP for design containing GmSeed12 promoter (Table 10, plasmid ID AR07-26).

TABLE 10
Summary of RNA and protein quantification of exemplary OVAL constructs.

| | | | | | | | # of | # of |
|---|---|---|---|---|---|---|---|---|
| | | | | # of all | # of all | # of | seeds | seeds |
| | | | | plants | seeds | seeds | with | with |
| | | | | analyzed | analyzed | below | 0.023- | over |
| Construct | | Highest | Max | for | for | detection | 1% | 1% |
| ID | Details | RNA level | % TSP | ELISA | ELISA | level | TSP | TSP |
| AR07-22 | BnNap:sig11:OOVAL1: | 0.002 | 0.000 | 3 | 31 | 31* | 0* | 0* |
| | KDEL:nos | | | | | | | |
| AR07-23 | GmSeed2:sig2:OOVAL1: | 0.03 | 0.004 | 4 | 32 | 32* | 0* | 0* |
| | KDEL:nos | | | | | | | |
| AR07-25 | GmSeed12:coixss:OOVAL1: | 0.1 | 0.009 | 4 | 32 | 32 | 0 | 0 |
| | KDEL:nos | | | | | | | |
| AR07-26 | GmSeed12:sig12:OOVAL1: | 0.06 | 0.08 | 3 | 24 | 21 | 3 | 0 |
| | KDEL:nos | | | | | | | |
| AR07-27 | PvPhas:arcUTR:sig10: | 0.08 | 0.06 | 5 | 40 | 38 | 2 | 0 |
| | OOVAL1:KDEL:arcT | | | | | | | |
| AR15-16 | GmSeed2:sig2:OOVAL2: | 0.11 | 2.74 | 10 | 80 | 37 | 36 | 7 |
| | KDEL:EUT:Rb7T | | | | | | | |
| AR15-17 | GmSeed2:sig2:OOVAL3: | 0.08 | 1.43 | 1 | 8 | 0 | 7 | 1 |
| | KDEL:EUT: Rb7T | | | | | | | |
| AR15-18 | GmSeed2:sig2:OOVAL4: | 0.04 | [symbol] | [symbol] | [symbol] | — | — | — |
| | KDEL:EUT:Rb7T | | | | | | | |
| AR15-19 | GmSeed2:sig2:OOVAL2: | 0.24 | 1.08 | 8 | 61 | 12 | 47 | 2 |
| | EUT:Rb7T | | | | | | | |
| AR15-20 | GmSeed2 (intron | 0.03 | [symbol] | [symbol] | [symbol] | — | — | — |
| | 1):sig2:OOVAL2:KDEL: | | | | | | | |
| | EUT:Rb7T | | | | | | | |
| AR15-21 | GmSeed2:sig2:OOVAL2 | 0.22 | 6.64 | 9 | 67 | 14 | 10 | 43 |
| | (intron | | | | | | | |
| | 1):KDEL:EUT:Rb7T | | | | | | | |
| AR15-22 | GmSeed2:sig2:OOVAL2 | 0.32 | 1.69 | 9 | 72 | 36 | 26 | 10 |
| | (intron | | | | | | | |
| | 2):KDEL:EUT:Rb7T | | | | | | | |
| AR15-23 | GmSeed2:ovalUTR:sig2: | 0.09 | [symbol] | [symbol] | [symbol] | — | — | — |
| | OOVAL2:KDEL:EUT:R | | | | | | | |
| | b7T | | | | | | | |
| AR15-24 | GmSeed2:glnB1UTR:sig2: | 0.12 | 2.28 | 10 | 80 | 47 | 25 | 8 |
| | 00VAL2:KDEL:EUT: | | | | | | | |
| | Rb7T | | | | | | | |
| AR15-38 | GmSeed2: Ubimonomer:sig2: | 0.05 | [symbol] | [symbol] | [symbol] | — | — | — |
| | OOVAL2:KDEL:EU | | | | | | | |
| | T:Rb7T | | | | | | | |

*Protein amount was determined using a western blot. No seeds were analyzed by ELISA for constructs AR07-22 and AR07-23. [symbol]
Plants were all discarded due to low RNA expression and not further analyzed.
(—) No data available.

TABLE 11
Sequences for exemplary introns, β-Lactoglobulin,
ovalbumin, signal peptide, terminators,
monomers, and promoters.

| | SEQ | |
|---|---|---|
| | ID | |
| | NO | Sequence |
| Introns | | |
| Intron 1 | 679 | GTTCGTTATCTACCACCGTTCTATGGATTTTATTCCTTCTATTCG |
| | | TGTTTATTCTATTGGTTTATGTTGCTTGCAATATGTTTTTTCTGA |
| | | ATCTGTCGTCGTTGTCTTCAATTTTATCCATGTTTCAGAGATCAA |
| | | TTTTGTTTGTGTAGTATGTGCTTATTCTTCTTCTTTTCGTTCGAG |
| | | TTGTTAATAACGGTGCTATGGTGTTTTCAAAAGTGTTTTTTTTAT |
| | | TACTTTTGATTTAAAGTTTTTTTGGTAAGGCTTTTATTTGCTTGT |
| | | TATATTCAAATCTTTGGATCCAGATCTTATATAAGTTTTTGGTTC |
| | | AAGAAAGTTTTTGGTTACTGATGAATAGATCTATTAACTGTTACT |
| | | TTAATCGATTCAAGCTAAAGTTTTTTGGTTACTGATGAATAGATC |
| | | TATTATCTGTTACTTTTAATCGGTTCAAGCTCAAGTTTTTTGGTT |
| | | ACTGATGAATAGATCTATATACGTCACAGTGTGCTAAACATGCCC |
| | | TTGTTTTATCTCGATCTTATGTATGGGAGTGCCATAAATTTTGTT |
| | | ATGTCTATTTTTTTATCTGTTGGAATCATACTGAGTTTGATGCGT |
| | | TACGATTGAGCATACCTATTTTTGGGCTTGTTGTATGGTGGGTAT |
| | | TTAGATCTTAATCTTTTTATGCTTATGAAAGGTTTTGTAATGACA |
| | | AAGGTCTTAATGTTGTTAAACTTTTATTTTTACTTTATATGGTGT |
| | | GTTGATGTGTTATGGTTTTGACAACTTTTTTTTTTTCTGGATTTT |
| | | TGCAG |
| Intron 2 | 680 | GTAACCATATCTTTCATCTGTTATGTGACTACACATTGCTTCTCT |
| | | TTTTGTGTTCTGTCTCATTAATTGCGGTTTGTTACATGTTGTTTG |
| | | TAG |
| Intron 3 | 681 | GTAAGCAACCAACACACCATCTAATACGCTAGCAAATTCAATATT |
| | | ATCATTATCCTTATATTTGTTTCCGCGCTTGATTTTATAG |
| Intron 4 | 682 | GTTTGTATTTACTCAAATGTTGATCAGTAGTGTTTTTAGGACATT |
| | | GATTAAGAAACCCAAAAAATAATTATTTTTATTGAAACGCATAAA |
| | | TTTATACTAGCCGTGACTGTTTTTATGTCCTTATATGATCTTCGC |
| | | AATATATATTTTCTATTATAAGTTTCTTAACCAATGCACTAACTT |
| | | ACTGTTAACAAGACCTTATTATTAAACATCATCTATCACTTGGTT |
| | | AATTGTATTCATTTGATGCATGGTAATGCATTACATATATACAG |
| LG Codon | | |
| optimized | | |
| OLG1 | 683 | TTGATCGTAACACAGACTATGAAGGGTCTTGATATACAGAAGGTG |
| | | GCCGGGACTTGGTACAGTTTGGCAATGGCCGCATCCGACATCTCC |
| | | TTGTTGGACGCACAATCAGCCCCATTGCGTGTGTACGTAGAAGAG |
| | | CTTAAACCAACTCCCGAGGGGGATCTGGAAATTCTGCTCCAGAAA |
| | | TGGGAGAACGGTGAGTGCGCCCAGAAGAAGATCATCGCAGAGAAG |
| | | ACCAAAATTCCAGCAGTATTCAAAATCGACGCATTGAACGAAAAT |
| | | AAGGTGCTCGTACTGGACACTGATTATAAGAAGTATCTCCTTTTC |
| | | TGTATGGAGAACTCAGCAGAGCCTGAACAGAGTCTTGCCTGCCAA |
| | | TGCCTTGTTCGTACCCCAGAGGTAGATGATGAAGCTCTGGAAAAG |
| | | TTCGATAAGGCCCTTAAGGCTCTGCCTATGCACATTAGGCTTTCT |
| | | TTCAATCCAACTCAACTTGAGGAACAATGTCACATT |
| OLG1 | 684 | LIVTQTMKGLDIQKVAGTWYSLAMAASDISLLDAQSAPLRVYVEE |
| | | LKPTPEGDLEILLQKWENGECAQKKIIAEKTKIPAVFKIDALNEN |
| | | KVLVLDTDYKKYLLFCMENSAEPEQSLACQCLVRTPEVDDEALEK |
| | | FDKALKALPMHIRLSFNPTQLEEQCHI |
| OLG2 | 685 | CTTATTGTGACCCAAACCATGAAGGGCCTCGACATTCAAAAGGTT |
| | | GCCGGAACCTGGTACTCCCTTGCTATGGCTGCTTCCGATATCTCC |
| | | TTGCTCGATGCTCAATCCGCTCCACTTAGGGTGTACGTGGAAGAG |
| | | TTGAAGCCAACTCCAGAGGGCGATCTTGAGATCTTGCTTCAAAAG |
| | | TGGGAGAACGATGAGTGCGCCCAGAAGAAGATTATCGCCGAAAAG |
| | | ACCAAGATTCCCGCCGTGTTCAAGATCGATGCTCTCAACGAGAAC |
| | | AAGGTGCTCGTGCTCGATACCGACTACAAGAAGTACCTTCTCGTC |
| | | TGCATGGAAAACTCCGCTGAGCCAGAGCAATCTCTTGTTTGCCAA |
| | | TGCCTTGTGAGGACCCCAGAGGTTGACGATGAAGCTCTTGAGAAG |
| | | TTCGACAAGGCTCTCAAGGCTTTGCCTATGCACATCCGCCTTAGC |
| | | TTCAACCCAACTCAGCTTGAGGAACAGTGCCACATC |
| OLG2 | 686 | LIVTQTMKGLDIQKVAGTWYSLAMAASDISLLDAQSAPLRVYVEE |
| | | LKPTPEGDLEILLQKWENDECAQKKIIAEKTKIPAVFKIDALNEN |
| | | KVLVLDTDYKKYLLVCMENSAEPEQSLVCQCLVRTPEVDDEALEK |
| | | FDKALKALPMHIRLSFNPTQLEEQCHI |
| OLG3 | 687 | CTCATTGTTACACAAACCATGAAGGGTCTTGACATTCAGAAGGTT |
| | | GCTGGGACATGGTATTCACTAGCGATGGCTGCTTCTGATATCTCC |
| | | CTGTTGGATGCACAGTCTGCCCCCCTGAGAGTGTATGTTGAAGAA |
| | | CTGAAACCGACACCTGAAGGAGACTTGGAAATTTTACTCCAGAAA |
| | | TGGGAAAATGATGAGTGTGCCCAAAAGAAGATAATAGCCGAGAAG |
| | | ACCAAAATTCCTGCTGTGTTTAAGATTGATGCTTTGAATGAGAAC |
| | | AAAGTACTAGTCCTCGACACTGATTACAAGAAATACTTATTAGTG |
| | | TGCATGGAAAACAGCGCAGAGCCAGAACAATCACTTGTTTGTCAA |
| | | TGTTTGGTCCGTACTCCAGAGGTAGATGATGAAGCATTGGAGAAA |
| | | TTTGATAAAGCATTGAAGGCACTTCCAATGCATATAAGGCTTAGT |
| | | TTCAATCCTACTCAGCTTGAAGAGCAATGCCACATC |
| OLG3 | 688 | LIVTQTMKGLDIQKVAGTWYSLAMAASDISLLDAQSAPLRVYVEE |
| | | LKPTPEGDLEILLQKWENDECAQKKIIAEKTKIPAVFKIDALNEN |
| | | KVLVLDTDYKKYLLVCMENSAEPEQSLVCQCLVRTPEVDDEALEK |
| | | FDKALKALPMHIRLSFNPTQLEEQCHI |
| OLG4 | 689 | CTTATAGTAACTCAAACCATGAAGGGACTTGATATCCAAAAAGTT |
| | | GCAGGAACCTGGTACTCACTGGCTATGGCAGCTTCCGACATCTCC |
| | | TTGTTGGACGCACAATCCGCACCATTGCGCGTCTACGTTGAGGAG |
| | | TTGAAACCTACACCAGAGGGGGATCTTGAGATTTTGCTCCAGAAA |
| | | TGGGAGAACGACGAGTGTGCCCAGAAAAAAATTATAGCAGAGAAG |
| | | ACTAAAATTCCTGCTGTTTTTAAGATTGATGCCCTGAACGAGAAT |
| | | AAGGTACTGGTCCTCGACACTGATTATAAAAAGTATTTGCTGGTG |
| | | TGTATGGAGAACAGTGCTGAACCTGAACAGAGCCTGGTCTGTCAA |
| | | TGTCTTGTAAGGACACCTGAGGTTGATGACGAGGCACTTGAAAAA |
| | | TTCGACAAGGCCCTTAAGGCTCTGCCTATGCACATCCGTCTGAGT |
| | | TTCAACCCTACTCAGTTGGAGGAACAATGTCATATT |
| OLG4 | 690 | LIVTQTMKGLDIQKVAGTWYSLAMAASDISLLDAQSAPLRVYVEE |
| | | LKPTPEGDLEILLQKWENDECAQKKIIAEKTKIPAVFKIDALNEN |
| | | KVLVLDTDYKKYLLVCMENSAEPEQSLVCQCLVRTPEVDDEALEK |
| | | FDKALKALPMHIRLSFNPTQLEEQCHI |
| OLG2 | 691 | CTTATTGTGACCCAAACCATGAAGGGCCTCGACATTCAAAAGGTT |
| (intron 1) | | CGTTATCTACCACCGTTCTATGGATTTTATTCCTTCTATTCGTGT |
| | | TTATTCTATTGGTTTATGTTGCTTGCAATATGTTTTTTCTGAATC |
| | | TGTCGTCGTTGTCTTCAATTTTATCCATGTTTCAGAGATCAATTT |
| | | TGTTTGTGTAGTATGTGCTTATTCTTCTTCTTTTCGTTCGAGTTG |
| | | TTAATAACGGTGCTATGGTGTTTTCAAAAGTGTTTTTTTTATTAC |
| | | TTTTGATTTAAAGTTTTTTTGGTAAGGCTTTTATTTGCTTGTTAT |
| | | ATTCAAATCTTTGGATCCAGATCTTATATAAGTTTTTGGTTCAAG |
| | | AAAGTTTTTGGTTACTGATGAATAGATCTATTAACTGTTACTTTA |
| | | ATCGATTCAAGCTAAAGTTTTTTGGTTACTGATGAATAGATCTAT |
| | | TATCTGTTACTTTTAATCGGTTCAAGCTCAAGTTTTTTGGTTACT |
| | | GATGAATAGATCTATATACGTCACAGTGTGCTAAACATGCCCTTG |
| | | TTTTATCTCGATCTTATGTATGGGAGTGCCATAAATTTTGTTATG |
| | | TCTATTTTTTTATCTGTTGGAATCATACTGAGTTTGATGCGTTAC |
| | | GATTGAGCATACCTATTTTTGGGCTTGTTGTATGGTGGGTATTTA |
| | | GATCTTAATCTTTTTATGCTTATGAAAGGTTTTGTAATGACAAAG |
| | | GTCTTAATGTTGTTAAACTTTTATTTTTACTTTATATGGTGTGTT |
| | | GATGTGTTATGGTTTTGACAACTTTTTTTTTTTCTGGATTTTTGC |
| | | AGGTTGCCGGAACCTGGTACTCCCTTGCTATGGCTGCTTCCGATA |
| | | TCTCCTTGCTCGATGCTCAATCCGCTCCACTTAGGGTGTACGTGG |
| | | AAGAGTTGAAGCCAACTCCAGAGGGCGATCTTGAGATCTTGCTTC |
| | | AAAAGTGGGAGAACGATGAGTGCGCCCAGAAGAAGATTATCGCCG |
| | | AAAAGACCAAGATTCCCGCCGTGTTCAAGATCGATGCTCTCAACG |
| | | AGAACAAGGTGCTCGTGCTCGATACCGACTACAAGAAGTACCTTC |
| | | TCGTCTGCATGGAAAACTCCGCTGAGCCAGAGCAATCTCTTGTTT |
| | | GCCAATGCCTTGTGAGGACCCCAGAGGTTGACGATGAAGCTCTTG |
| | | AGAAGTTCGACAAGGCTCTCAAGGCTTTGCCTATGCACATCCGCC |
| | | TTAGCTTCAACCCAACTCAGCTTGAGGAACAGTGCCACATC |
| OLG2 | 692 | CTTATTGTGACCCAAACCATGAAGGGCCTCGACATTCAAAAGGTA |
| (intron 2) | | ACCATATCTTTCATCTGTTATGTGACTACACATTGCTTCTCTTTT |
| | | TGTGTTCTGTCTCATTAATTGCGGTTTGTTACATGTTGTTTGTAG |
| | | GTTGCCGGAACCTGGTACTCCCTTGCTATGGCTGCTTCCGATATC |
| | | TCCTTGCTCGATGCTCAATCCGCTCCACTTAGGGTGTACGTGGAA |
| | | GAGTTGAAGCCAACTCCAGAGGGCGATCTTGAGATCTTGCTTCAA |
| | | AAGTGGGAGAACGATGAGTGCGCCCAGAAGAAGATTATCGCCGAA |
| | | AAGACCAAGATTCCCGCCGTGTTCAAGATCGATGCTCTCAACGAG |
| | | AACAAGGTGCTCGTGCTCGATACCGACTACAAGAAGTACCTTCTC |
| | | GTCTGCATGGAAAACTCCGCTGAGCCAGAGCAATCTCTTGTTTGC |
| | | CAATGCCTTGTGAGGACCCCAGAGGTTGACGATGAAGCTCTTGAG |
| | | AAGTTCGACAAGGCTCTCAAGGCTTTGCCTATGCACATCCGCCTT |
| | | AGCTTCAACCCAACTCAGCTTGAGGAACAGTGCCACATC |
| Oval Codon | | |
| optimized | | |
| OOVAL1 | 693 | GGTAGCATTGGGGCTGCTTCTATGGAATTTTGTTTCGATGTCTTT |
| | | AAAGAACTTAAGGTACACCATGCAAATGAGAACATTTTCTACTGT |
| | | CCCATCGCTATAATGTCTGCACTTGCAATGGTTTACCTTGGGGCT |
| | | AAAGACAGTACTCGTACACAAATAAATAAAGTAGTGAGATTCGAT |
| | | AAGTTGCCTGGGTTCGGGGATTCTATCGAAGCTCAATGTGGGACC |
| | | AGTGTTAACGTACATAGCTCCTTGCGCGATATCTTGAATCAAATA |
| | | ACAAAGCCTAATGATGTATACTCATTTTCATTGGCCTCTCGCTTG |
| | | TATGCCGAGGAAAGATACCCCATTCTGCCAGAATACCTTCAGTGC |
| | | GTCAAGGAACTCTACCGCGGAGGACTCGAGCCCATAAATTTCCAG |
| | | ACTGCAGCAGACCAGGCCAGGGAGCTGATTAACTCTTGGGTAGAG |
| | | AGCCAGACAAATGGCATAATCAGGAATGTGCTGCAGCCATCATCA |
| | | GTTGATTCACAAACAGCTATGGTGCTGGTTAATGCAATCGTCTTC |
| | | AAAGGGTTGTGGGAAAAGGCTTTTAAGGACGAAGATACTCAAGCT |
| | | ATGCCTTTCCGTGTAACAGAGCAAGAAAGCAAGCCTGTACAAATG |
| | | ATGTATCAGATTGGTCTGTTTCGTGTTGCCTCTATGGCTTCAGAG |
| | | AAAATGAAGATACTCGAACTTCCCTTCGCATCAGGGACTATGAGC |
| | | ATGTTGGTTTTGTTGCCTGATGAGGTATCTGGTTTGGAACAGCTG |
| | | GAATCAATAATCAATTTCGAGAAGTTGACAGAATGGACCAGTTCT |
| | | AATGTTATGGAAGAGCGTAAGATAAAAGTATATTTGCCTCGTATG |
| | | AAAATGGAAGAAAAGTACAATTTGACCAGCGTTTTGATGGCTATG |
| | | GGCATCACTGACGTTTTTTCATCTTCTGCTAATCTCAGCGGCATA |
| | | TCCAGCGCAGAGAGCCTCAAAATATCCCAAGCCGTCCATGCTGCA |
| | | CATGCAGAGATAAATGAGGCTGGTAGGGAAGTGGTCGGGAGCGCT |
| | | GAAGCTGGGGTAGATGCAGCCAGTGTAAGTGAAGAGTTCAGGGCT |
| | | GACCATCCCTTCCTGTTCTGCATTAAGCACATTGCAACTAACGCA |
| | | GTACTCTTTTTTGGACGTTGCGTGAGCCCC |
| OOVAL1 | 694 | GSIGAASMEFCFDVFKELKVHHANENIFYCPIAIMSALAMVYLGA |
| | | KDSTRTQINKVVRFDKLPGFGDSIEAQCGTSVNVHSSLRDILNQI |
| | | TKPNDVYSFSLASRLYAEERYPILPEYLQCVKELYRGGLEPINFQ |
| | | TAADQARELINSWVESQTNGIIRNVLQPSSVDSQTAMVLVNAIVF |
| | | KGLWEKAFKDEDTQAMPFRVTEQESKPVQMMYQIGLFRVASMASE |
| | | KMKILELPFASGTMSMLVLLPDEVSGLEQLESIINFEKLTEWTSS |
| | | NVMEERKIKVYLPRMKMEEKYNLTSVLMAMGITDVFSSSANLSGI |
| | | SSAESLKISQAVHAAHAEINEAGREVVGSAEAGVDAASVSEEFRA |
| | | DHPFLFCIKHIATNAVLFFGRCVSP |
| OOVAL2 | 695 | GGATCAATTGGCGCCGCATCTATGGAGTTCTGCTTCGATGTTTTT |
| | | AAAGAGCTTAAAGTGCACCATGCCAACGAGAATATCTTCTATTGC |
| | | CCAATTGCCATTATGTCTGCCCTTGCTATGGTGTACTTGGGTGCT |
| | | AAAGACTCTACTAGGACCCAGATAAACAAGGTAGTCAGATTCGAC |
| | | AAGCTGCCTGGGTTTGGCGACTCTATTGAAGCTCAGTGTGGTACT |
| | | TCTGTTAATGTCCACTCATCCCTCCGCGACATACTTAATCAAATT |
| | | ACAAAACCAAATGATGTGTACTCATTTAGTCTGGCCAGCCGTTTG |
| | | TACGCAGAGGAACGCTACCCTATCCTGCCAG |
| | | AGTATTTGCAATGTGTGAAGGAACTTTACAGGGGTGGGCTTGAGC |
| | | CAATAAACTTTCAAACAGCAGCCGACCAAGCTAGGGAGCTTATCA |
| | | ATTCTTGGGTCGAGAGCCAAACTAACGGAATCATCCGCAACGTCC |
| | | TCCAGCCAAGTTCCGTTGATTCCCAGACCGCTATGGTACTTGTGA |
| | | ATGCCATTGTCTTCAAGGGGCTTTGGGAGAAGGCATTTAAAGACG |
| | | AGGACACTCAGGCAATGCCCTTTCGTGTGACCGAGCAGGAGTCAA |
| | | AACCTGTTCAAATGATGTACCAAATTGGGCTGTTCAGAGTTGCTA |
| | | GTATGGCCTCTGAGAAAATGAAGATCCTTGAACTCCCATTTGCCT |
| | | CCGGGACAATGTCTATGCTTGTCCTCCTGCCAGATGAAGTCAGTG |
| | | GGCTCGAACAGCTCGAAAGCATAATAAACTTTGAGAAACTTACCG |
| | | AATGGACTTCTTCCAATGTTATGGAGGAGCGTAAAATTAAGGTCT |
| | | ATCTGCCCCGCATGAAAATGGAGGAAAAGTATAATCTCACTAGCG |
| | | TCCTCATGGCTATGGGAATTACTGATGTATTCTCCTCTAGCGCTA |
| | | ATCTGAGTGGAATCTCCAGCGCCGAGTCTCTCAAGATAAGCCAGG |
| | | CCGTGCACGCTGCTCATGCTGAAATCAACGAAGCCGGCAGAGAGG |
| | | TGGTGGGGTCAGCTGAGGCAGGTGTAGATGCAGCCAGTGTCTCTG |
| | | AGGAATTTAGAGCCGATCACCCTTTCCTTTTTTGCATTAAACATA |
| | | TCGCTACAAATGCCGTTTTGTTTTTCGGTCGTTGCGTTAGTCCA |
| OOVAL2 | 696 | GSIGAASMEFCFDVFKELKVHHANENIFYCPIAIMSALAMVYLGA |
| | | KDSTRTQINKVVRFDKLPGFGDSIEAQCGTSVNVHSSLRDILNQI |
| | | TKPNDVYSFSLASRLYAEERYPILPEYLQCVKELYRGGLEPINFQ |
| | | TAADQARELINSWVESQTNGIIRNVLQPSSVDSQTAMVLVNAIVF |
| | | KGLWEKAFKDEDTQAMPFRVTEQESKPVQMMYQIGLFRVASMASE |
| | | KMKILELPFASGTMSMLVLLPDEVSGLEQLESIINFEKLTEWTSS |
| | | NVMEERKIKVYLPRMKMEEKYNLTSVLMAMGITDVFSSSANLSGI |
| | | SSAESLKISQAVHAAHAEINEAGREVVGSAEAGVDAASVSEEFRA |
| | | DHPFLFCIKHIATNAVLFFGRCVSP |
| OOVAL3 | 697 | GGGTCCATCGGTGCCGCCTCAATGGAATTCTGCTTTGACGTCTTC |
| | | AAGGAACTTAAGGTACATCATGCCAACGAGAATATTTTTTACTGT |
| | | CCAATAGCTATCATGAGTGCACTTGCTATGGTGTACCTTGGAGCC |
| | | AAAGACTCAACCCGTACCCAGATCAACAAGGTGGTCCGCTTTGAC |
| | | AAACTGCCAGGGTTTGGGGATTCTATTGAGGCCCAATGCGGAACA |
| | | AGTGTGAACGTCCACTCTAGCTTGCGCGATATACTTAATCAAATA |
| | | ACTAAACCAAATGATGTGTATTCATTCTCTCTCGCCAGCAGACTG |
| | | TACGCAGAAGAAAGGTATCCCATTCTCCCCGAGTACCTCCAATGC |
| | | GTAAAGGAGTTGTACAGAGGCGGCCTGGAACCCATAAATTTCCAA |
| | | ACTGCCGCAGATCAGGCTCGTGAGCTGATAAATTCATGGGTCGAG |
| | | AGCCAAACTAACGGTATCATTCGTAATGTCCTTCAACCCTCAAGT |
| | | GTGGACAGTCAGACAGCCATGGTCCTCGTCAATGCTATAGTCTTC |
| | | AAAGGCCTGTGGGAAAAGACCTTTAAGGATGAAGATACTCAAGCA |
| | | ATGCCCTTTAGAGTCACAGAGCAAGAAAGCAAACCCGTGCAAATG |
| | | ATGTATCAAATCGGGCTCTTTCGTGTTGCATCCATGGCATCTGAA |
| | | AAGATGAAGAT |
| | | ATTGGAACTCCCCTTCGCCTCTGGAACAATGAGTATGTTGGTACT |
| | | TCTGCCCGATGAGGTCTCTGGGTTGGAACAGCTTGAATCTATTAT |
| | | TAACTTCGAGAAACTGACCGAGTGGACTAGTAGTAATGTCATGGA |
| | | GGAGAGAAAGATTAAGGTTTATTTGCCACGCATGAAGATGGAAGA |
| | | GAAATATAACTTGACATCTGTACTGATGGCAATGGGTATAACCGA |
| | | CGTATTTAGCAGTAGCGCCAATCTGTCAGGGATTTCTTCAGCCGA |
| | | AAGTCTCAAGATTTCTCAGGCAGTTCACGCAGCCCATGCAGAGAT |
| | | AAACGAAGCAGGCCGCGAAGTTGTCGGATCTGCAGAAGCCGGCGT |
| | | GGATGCAGCCAGTGTCTCCGAAGAGTTCAGAGCAGACCACCCTTT |
| | | CCTCTTCTGCATTAAGCACATCGCAACCAACGCAGTACTTTTTTT |
| | | CGGACGTTGCGTGTCCCCA |
| OOVAL3 | 698 | GSIGAASMEFCFDVFKELKVHHANENIFYCPIAIMSALAMVYLGA |
| | | KDSTRTQINKVVRFDKLPGFGDSIEAQCGTSVNVHSSLRDILNQI |
| | | TKPNDVYSFSLASRLYAEERYPILPEYLQCVKELYRGGLEPINFQ |
| | | TAADQARELINSWVESQTNGIIRNVLQPSSVDSQTAMVLVNAIVF |
| | | KGLWEKAFKDEDTQAMPFRVTEQESKPVQMMYQIGLFRVASMASE |
| | | KMKILELPFASGTMSMLVLLPDEVSGLEQLESIINFEKLTEWTSS |
| | | NVMEERKIKVYLPRMKMEEKYNLTSVLMAMGITDVFSSSANLSGI |
| | | SSAESLKISQAVHAAHAEINEAGREVVGSAEAGVDAASVSEEFRA |
| | | DHPFLFCIKHIATNAVLFFGRCVSP |
| OOVAL4 | 699 | GGTTCAATAGGAGCTGCGTCTATGGAGTTTTGTTTTGATGTCTTT |
| | | AAGGAACTCAAAGTCCACCACGCCAATGAAAATATTTTCTATTGC |
| | | CCTATTGCAATCATGAGTGCGCTAGCCATGGTTTACTTGGGTGCA |
| | | AAAGACAGTACGCGTACTCAAATAAACAAGGTTGTTCGCTTTGAC |
| | | AAGCTTCCTGGATTTGGAGATAGTATTGAAGCACAATGTGGAACT |
| | | AGCGTAAACGTCCACAGCTCATTGAGGGACATTCTTAACCAAATT |
| | | ACCAAGCCAAATGATGTATATAGTTTTTCCTTGGCATCACGACTG |
| | | TATGCAGAAGAAAGATATCCTATCCTCCCGGAATATCTTCAGTGC |
| | | GTGAAAGAATTATACAGAGGTGGGCTAGAGCCAATCAATTTTCAA |
| | | ACCGCTGCTGATCAAGCTCGCGAGTTGATTAACTCATGGGTTGAG |
| | | AGCCAGACAAATGGGATAATAAGAAATGTTCTTCAACCATCTAGT |
| | | GTGGACTCTCAAACAGCAATGGTGCTCGTCAATGCGATAGTTTTT |
| | | AAAGGCTTGTGGGAGAAAACATTCAAAGATGAGGATACTCAGGCA |
| | | ATGCCATTCCGTGTAACTGAACAGGAATCTAAGCCTGTTCAAATG |
| | | ATGTATCAGATTGGTTTGTTCAGAGTTGCCTCTATGGCATCTGAA |
| | | AAAATGAAAATTTTGGAGCTTCCATTTGCTAGTGGAACAATGTCA |
| | | ATGTTAGTTTTACTGCCTGATGAAGTGTCCGGTTTAGAACAATTG |
| | | GAATCAATTATCAACTTTGAAAAGTTGACCGAGTGGACTTCCTCC |
| | | AATGTGATGGAGGAGAGGAAGATTAAGGTGTACCTTCCCAGAATG |
| | | AAGATGGAAGAGAAATATAACCTGACTTCGGTCCTAATGGCTATG |
| | | GGGATCACAGATGTGTTTTCTTCCTCGGCAAACCTTTCAGGCATA |
| | | TCAAGCGCCGAGTCATTGAAAATTTCACAGGCTGTTCATGCAGCT |
| | | CATGCTGAAATCAATGAGGCCGGGCGGGAGGTTGTGGGCAGTGCT |
| | | GAAGCTGGAGTTGATGCTGCCTCAGTGTCTGAGGAATTTAGAGCA |
| | | GATCATCCTTTCCTCTTCTGCATTAAGCATATTGCTACCAATGCT |
| | | GTACTGTTCTTCGGTAGGTGTGTTAGCCCC |
| OOVAL4 | 700 | GSIGAASMEFCFDVFKELKVHHANENIFYCPIAIMSALAMVYLGA |
| | | KDSTRTQINKVVRFDKLPGFGDSIEAQCGTSVNVHSSLRDILNQI |
| | | TKPNDVYSFSLASRLYAEERYPILPEYLQCVKELYRGGLEPINFQ |
| | | TAADQARELINSWVESQTNGIIRNVLQPSSVDSQTAMVLVNAIVF |
| | | KGLWEKTFKDEDTQAMPFRVTEQESKPVQMMYQIGLFRVASMASE |
| | | KMKILELPFASGTMSMLVLLPDEVSGLEQLESIINFEKLTEWTSS |
| | | NVMEERKIKVYLPRMKMEEKYNLTSVLMAMGITDVFSSSANLSGI |
| | | SSAESLKISQAVHAAHAEINEAGREVVGSAEAGVDAASVSEEFRA |
| | | DHPFLFCIKHIATNAVLFFGRCVSP |
| OOVAL2 | 701 | GGATCAATTGGCGCCGCATCTATGGAGTTCTGCTTCGATGTTTTT |
| (intron 1) | | AAAGAGCTTAAAGTGCACCATGCCAACGAGAATATCTTCTATTGC |
| | | CCAATTGCCATTATGTCTGCCCTTGCTATGGTGTACTTGGGTGCT |
| | | AAAGACTCTACTAGGACCCAGATAAACAAGGTTCGTTATCTACCA |
| | | CCGTTCTATGGATTTTATTCCTTCTATTCGTGTTTATTCTATTGG |
| | | TTTATGTTGCTTGCAATATGTTTTTTCTGAATCTGTCGTCGTTGT |
| | | CTTCAATTTTATCCATGTTTCAGAGATCAATTTTGTTTGTGTAGT |
| | | ATGTGCTTATTCTTCTTCTTTTCGTTCGAGTTGTTAATAACGGTG |
| | | CTATGGTGTTTTCAAAAGTGTTTTTTTTATTACTTTTGATTTAAA |
| | | GTTTTTTTGGTAAGGCTTTTATTTGCTTGTTATATTCAAATCTTT |
| | | GGATCCAGATCTTATATAAGTTTTTGGTTCAAGAAAGTTTTTGGT |
| | | TACTGATGAATAGATCTATTAACTGTTACTTTAATCGATTCAAGC |
| | | TAAAGTTTTTTGGTTACTGATGAATAGATCTATTATCTGTTACTT |
| | | TTAATCGGTTCAAGCTCAAGTTTTTTGGTTACTGATGAATAGATC |
| | | TATATACGTCACAGTGTGCTAAACATGCCCTTGTTTTATCTCGAT |
| | | CTTATGTATGGGAGTGCCATAAATTTTGTTATGTCTATTTTTTTA |
| | | TCTGTTGGAATCATACTGAGTTTGATGCGTTACGATTGAGCATAC |
| | | CTATTTTTGGGCTTGTTGTATGGTGGGTATTTAGATCTTAATCTT |
| | | TTTATGCTTATGAAAGGTTTTGTAATGACAAAGGTCTTAATGTTG |
| | | TTAAACTTTTATTTTTACTTTATATGGTGTGTTGATGTGTTATGG |
| | | TTTTGACAACTTTTTTTTTTTCTGGATTTTTGCAGGTAGTCAGAT |
| | | TCGACAAGCTGCCTGGGTTTGGCGACTCTATTGAAGCTCAGTGTG |
| | | GTACTTCTGTTAATGTCCACTCATCCCTCCGCGACATACTTAATC |
| | | AAATTACAAAACCAAATGATGTGTACTCATTTAGTCTGGCCAGCC |
| | | GTTTGTACGCAGAGGAACGCTACCCTATCCTGCCAGAGTATTTGC |
| | | AATGTGTGAAGGAACTTTACAGGGGTGGGCTTGAGCCAATAAACT |
| | | TTCAAACAGCAGCCGACCAAGCTAGGGAGCTTATCAATTCTTGGG |
| | | TCGAGAGCCAAACTAACGGAATCATCCGCAACGTCCTCCAGCCAA |
| | | GTTCCGTTGATTCCCAGACCGCTATGGTACTTGTGAATGCCATTG |
| | | TCTTCAAGGGGCTTTGGGAGAAGGCATTTAAAGACGAGGACACTC |
| | | AGGCAATGCCCTTTCGTGTGACCGAGCAGGAGTCAAAACCTGTTC |
| | | AAATGATG |
| OOVAL2 | 702 | TACCAAATTGGGCTGTTCAGAGTTGCTAGTATGGCCTCTGAGAAA |
| (intron 2) | | ATGAAGATCCTTGAACTCCCATTTGCCTCCGGGACAATGTCTATG |
| | | CTTGTCCTCCTGCCAGATGAAGTCAGTGGGCTCGAACAGCTCGAA |
| | | AGCATAATAAACTTTGAGAAACTTACCGAATGGACTTCTTCCAAT |
| | | GTTATGGAGGAGCGTAAAATTAAGGTCTATCTGCCCCGCATGAAA |
| | | ATGGAGGAAAAGTATAATCTCACTAGCGTCCTCATGGCTATGGGA |
| | | ATTACTGATGTATTCTCCTCTAGCGCTAATCTGAGTGGAATCTCC |
| | | AGCGCCGAGTCTCTCAAGATAAGCCAGGCCGTGCACGCTGCTCAT |
| | | GCTGAAATCAACGAAGCCGGCAGAGAGGTGGTGGGGTCAGCTGAG |
| | | GCAGGTGTAGATGCAGCCAGTGTCTCTGAGGAATTTAGAGCCGAT |
| | | CACCCTTTCCTTTTTTGCATTAAACATATCGCTACAAATGCCGTT |
| | | TTGTTTTTCGGTCGTTGCGTTAGTCCAGGATCAATTGGCGCCGCA |
| | | TCTATGGAGTTCTGCTTCGATGTTTTTAAAGAGCTTAAAGTGCAC |
| | | CATGCCAACGAGAATATCTTCTATTGCCCAATTGCCATTATGTCT |
| | | GCCCTTGCTATGGTGTACTTGGGTGCTAAAGACTCTACTAGGACC |
| | | CAGATAAACAAGGTAACCATATCTTTCATCTGTTATGtgactaca |
| | | cattgcttctctttttgtgttctgtctcattaattgCGGTTTGTT |
| | | ACATGTTGTTTGTAGGTAGTCAGATTCGACAAGCTGCCTGGGTTT |
| | | GGCGACTCTATTGAAGCTCAGTGTGGTACTTCTGTTAATGTCCAC |
| | | TCATCCCTCCGCGACATACTTAATCAAATTACAAAACCAAATGAT |
| | | GTGTACTCATTTAGTCTGGCCAGCCGTTTGTACGCAGAGGAACGC |
| | | TACCCTATCCTGCCAGAGTATTTGCAATGTGTGAAGGAACTTTAC |
| | | AGGGGTGGGCTTGAGCCAATAAACTTTCAAACAGCAGCCGACCAA |
| | | GCTAGGGAGCTTATCAATTCTTGGGTCGAGAGCCAAACTAACGGA |
| | | ATCATCCGCAACGTCCTCCAGCCAAGTTCCGTTGATTCCCAGACC |
| | | GCTATGGTACTTGTGAATGCCATTGTCTTCAAGGGGCTTTGGGAG |
| | | AAGGCATTTAAAGACGAGGACACTCAGGCAATGCCCTTTCGTGTG |
| | | ACCGAGCAGGAGTCAAAACCTGTTCAAATGATGTACCAAATTGGG |
| | | CTGTTCAGAGTTGCTAGTATGGCCTCTGAGAAAATGAAGATCCTT |
| | | GAACTCCCATTTGCCTCCGGGACAATGTCTATGCTTGTCCTCCTG |
| | | CCAGATGAAGTCAGTGGGCTCGAACAGCTCGAAAGCATAATAAAC |
| | | TTTGAGAAACTTACCGAATGGACTTCTTCCAATGTTATGGAGGAG |
| | | CGTAAAATTAAGGTCTATCTGCCCCGCATGAAAATGGAGGAAAAG |
| | | TATAATCTCACTAGCGTCCTCATGGCTATGGGAATTACTGATGTA |
| | | TTCTCCTCTAGCGCTAATCTGAGTGGAATCTCCAGCGCCGAGTCT |
| | | CTCAAGATAAGCCAGGCCGTGCACGCTGCTCATGCTGAAATCAAC |
| | | GAAGCCGGCAGAGAGGTGGTGGGGTCAGCTGAGGCAGGTGTAGAT |
| | | GCAGCCAGTGTCTCTGAGGAATTTAGAGCCGATCACCCTTTCCTT |
| | | TTTTGCATTAAACATATCGCTACAAATGCCGTTTTGTTTTTCGGT |
| | | CGTTGCGTTAGTCCA |
| Promoters | | |
| GmSeed2 | 703 | AACACAAGCTTCAAGTTTTAAAAGGAAAAATGTCAGCCAAAAACT |
| | | TTAAATAAAATGGTAACAAGGAAATTATTCAAAAATTACAAACCT |
| | | CGTCAAAATAGGAAAGAAAAAAAGTTTAGGGATTTAGAAAAAACA |
| | | TCAATCTAGTTCCACCTTATTTTATAGAGAGAAGAAACTAATATA |
| | | TAAGAACTAAAAAACAGAAGAATAGAAAAAAAAAGTATTGACAGG |
| | | AAAGAAAAAGTAGCTGTATGCTTATAAGTACTTTGAGGATTTGAA |
| | | TTCTCTCTTATAAAACACAAACACAATTTTTAGATTTTATTTAAA |
| | | TAATCATCAATCCGATTATAATTATTTATATATTTTTCTATTTTC |
| | | AAAGAAGTAAATCATGAGCTTTTCCAACTCAACATCTATTTTTTT |
| | | TCTCTCAACCTTTTTCACATCTTAAGTAGTCTCACCCTTTATATA |
| | | TATAACTTATTTCTTACCTTTTACATTATGTAACTTTTATCACCA |
| | | AAACCAACAACTTTAAAATTTTATTAAATAGACTCCACAAGTAAC |
| | | TTGACACTCTTACATTCATCGACATTAACTTTTATCTGTTTTATA |
| | | AATATTATTGTGATATAATTTAATCAAAATAACCACAAACTTTCA |
| | | TAAAAGGTTCTTATTAAGCATGGCATTTAATAAGCAAAAACAACT |
| | | CAATCACTTTCATATAGGAGGTAGCCTAAGTACGTACTCAAAATG |
| | | CCAACAAATAAAAAAAAAGTTGCTTTAATAATGCCAAAACAAATT |
| | | AATAAAACACTTACAACACCGGATTTTTTTTAATTAAAATGTGCC |
| | | ATTTAGGATAAATAGTTAATATTTTTAATAATTATTTAAAAAGCC |
| | | GTATCTACTAAAATGATTTTTATTTGGTTGAAAATATTAATATGT |
| | | TTAAATCAACACAATCTATCAAAATTAAACTAAAAAAAAAATAAG |
| | | TGTACGTGGTTAACATTAGTACAGTAATATAAGAGGAAAATGAGA |
| | | AATTAAGAAATTGAAAGCGAGTCTAATTTTTAAATTATGAACCTG |
| | | CATATATAAAAGGAAAGAAAGAATCCAGGAAGAAAAGAAATGAAA |
| | | CCATGCATGGTCCCCTCGTCATCACGAGTTTCTGCCATTTGCAAT |
| | | AGAAACACTGAAACACCTTTCTCTTTGTCACTTAATTGAGATGCC |
| | | GAAGCCACCTCACACCATGAACTTCATGAGGTGTAGCACCCAAGG |
| | | CTTCCATAGCCATGCATACTGAAGAATGTCTCAAGCTCAGCACCC |
| | | TACTTCTGTGACGTGTCCCTCATTCACCTTCCTCTCTTCCCTATA |
| | | AATAACCACGCCTCAGGTTCTCCGCTTCACAACTCAAACATTCTC |
| | | TCCATTGGTCCTTAAACACTCATCAGTCATCACC |
| GmSeed12 | 704 | CAACTATTATCGCATGATGATGTACGTTAAGTCATCATCATCTTT |
| | | AACTTTATATATTGTTAAAAGTAGAAAAAATAGGTGATGCATTAT |
| | | AAAATAATTTTATAACATCATTTAATTATAAATTATTTATAATAA |
| | | ATATTTGAGTTTTTATAGTAATTACCTAAACAATTATATCAAGAC |
| | | TAATGCCTGATTAGTTGACATGACGAAATTAAACTCATAAAAGTA |
| | | AAGATGTTTATGTGGAAAACTCTTATACAATTGAGCGGACTTTTT |
| | | TCCATGGTAGTTCAGTTTTCTTCTATTCAATTTATTTTTTTGGTT |
| | | TCCGCTCAGAATAAGAATAATTTGATAAATTCATTTTTAGGCAAT |
| | | TAAGAATATTTATTTGACTAACTTTTTAATTGAAATAAATTTACA |
| | | ATAAATACTCAATTTATCTTTCACAATCAAAAGATTGAGATGTTG |
| | | TAAGATCTCCGATAATATACTTATATCTTTTCATTTATTACGTTT |
| | | TCAAATTTGAATTTTAATGTGTGTTGTAAGTATAAATTTAAAATA |
| | | AAAATAAAAACAATTATTATATCAAAATGGCAAAAACATTTAATA |
| | | CGTATTATTTAAGAAAAAAATATGTAATAATATATTTATATTTTA |
| | | ATATCTATTCTTATGTATTTTTTAAAAATCTATTATATATTGATC |
| | | AACTAAAATATTTTTATATCTACACTTATTTTGCATTTTTATCAA |
| | | TTTTCTTGCGTTTTTTGGCATATTTAATAATGACTATTCTTTAAT |
| | | AATCAATCATTATTCTTACATGGTACATATTGTTGGAACCATATG |
| | | AAGTGTCCATTGCATTTGACTATGTGGATAGTGTTTTGATCCAGG |
| | | CCTCCATTTGCCGCTTATTAATTAATTTGGTAACAGTCCGTACTA |
| | | ATCAGTTACTTATCCTTCCTCCATCATAATTAATCTTGGTAGTCT |
| | | CGAATGCCACAACACTGACTAGTCTCTTGGATCATAAGAAAAAGC |
| | | CAAGGAACAAAAGAAGACAAAACACAATGAGAGTATCCTTTGCAT |
| | | AGCAATGTCTAAGTTCATAAAATTCAAACAAAAACGCAATCACAC |
| | | ACAGTGGACATCACTTATCCACTAGCTGATCAGGATCGCCGCGTC |
| | | AAGAAAAAAAAACTGGACCCCAAAAGCCATGCACAACAACACGTA |
| | | CTCACAAAGGTGTCAATCGAGCAGCCCAAAACATTCACCAACTCA |
| | | ACCCATCATGAGCCCACACATTTGTTGTTTCTAACCCAACCTCAA |
| | | ACTCGTATTCTCTTCCGCCACCTCATTTTTGTTTATTTCAACACC |
| | | CGTCAAACTGCATGCCACCCCGTGGCCAAATGTCCATGCATGTTA |
| | | ACAAGACCTATGACTATAAATATCTGCAATCTCGGCCCAGGTTTT |
| | | CATCATCAAGAACCAGTTCAATATCCTAGTACACCGTATTAAAGA |
| | | ATTTAAGATATACT |
| PvPhas | 705 | CATTGTACTCCCAGTATCATTATAGTGAAAGTTTTGGCTCTCTCG |
| | | CCGGTGGTTTTTTACCTCTATTTAAAGGGGTTTTCCACCTAAAAA |
| | | TTCTGGTATCATTCTCACTTTACTTGTTACTTTAATTTCTCATAA |
| | | TCTTTGGTTGAAATTATCACGCTTCCGCACACGATATCCCTACAA |
| | | ATTTATTATTTGTTAAACATTTTCAAACCGCATAAAATTTTATGA |
| | | AGTCCCGTCTATCTTTAATGTAGTCTAACATTTTCATATTGAAAT |
| | | ATATAATTTACTTAATTTTAGCGTTGGTAGAAAGCATAATGATTT |
| | | ATTCTTATTCTTCTTCATATAAATGTTTAATATACAATATAAACA |
| | | AATTCTTTACCTTAAGAAGGATTTCCCATTTTATATTTTAAAAAT |
| | | ATATTTATCAAATATTTTTCAACCACGTAAATCACATAATAATAA |
| | | GTTGTTTCAAAAGTAATAAAATTTAACTCCATAATTTTTTTATTT |
| | | GACTGATCTTAAAGCAACACCCAGTGACACAACTAGCCATTTTTT |
| | | TCTTTGAATAAAAAAATCCAATTATCATTGTATTTTTTTTATACA |
| | | ATGAAAATTTCACCAAACAATGATTTGTGGTATTTCTGAAGCAAG |
| | | TCATGTTATGCAAAATTCTATAATTCCCATTTGACACTACGGAAG |
| | | TAACTGAAGATCTGCTTTTACATGCGAGACACATCTTCTAAAGTA |
| | | ATTTTAATAATAGTTACTATATTCAAGATTTCATATATCAAATAC |
| | | TCAATATTACTTCTAAAAAATTAATTAGATATAATTAAAATATTA |
| | | CTTTTTTAATTTTAAGTTTAATTGTTGAATTTGTGACTATTGATT |
| | | TATTATTCTACTATGTTTAAATTGTTTTATAGGTAGTTTAAAGTA |
| | | AATATAAGTAATGTAGTAGAGTGTTAGAGTGTTACCCTAAACCAT |
| | | AAACTATAAGATTTATGGTGGACTAATTTTCATATATTTCTTATT |
| | | GCTTTTACCTTTTCTTGGTATGTAAGTCCGTAACTGGAATTACTG |
| | | TGGGTTGCCATGACACTCTGTGGTCTT |
| BnNap | 706 | TTGGTTCATGCATGGATGCTTGCGCAAGAAAAAGACAAAGAACAA |
| | | AGAAAAAAGACAAAACAGAGAGACAAAACGCAATCACACAACCAA |
| | | CTCAAATTAGTCACTGGCTGATCAAGATCGCCGCGTCCATGTATG |
| | | TCTAAATGCCATGCAAAGCAACACGTGCTTAACATGCACTTTAAA |
| | | TGGCTCACCCATCCCAACCCACTCACAAACACATTGCCTTTTTCT |
| | | TCATCATCACCACAACCACCTGTATATATTCATTCTCTTCCGCCA |
| | | CCTCAATTTCTTCACTTCAACACACGTCAACCTGCATATGCGTGT |
| | | CATCCCATGCCCAAATCTCCATGCATGTTCCTACCACCTTCTCTC |
| | | TTATATAATACCTATAAATACCTCTAATATCACTCACTTCTTTCA |
| | | TCATCCATCCATCCAGAGTACTACTACTCTACTACTATAATACCC |
| | | CAACCCAACTCATATTCAATACTACTCTACTCATCGGTGATTGAT |
| | | TCCTTTAAAGACTTATGTTTCTTATCTTGCTTCTGAGGCAAGTAT |
| | | TCAGTTACCAGTTACCACTTATATTCTGGACTTTCTGACTGCATC |
| | | CTCATTTTTCCAACATTTTAAATTTCACTATTGGCTGAATGCTTC |
| | | TTCTTTGAGGAAGAAACAATTCAGATGGCAGAAATGTATCAACCA |
| | | ATGCATATATACAAATGTACCTCTTGTTCTCAAAACATCTATCGG |
| | | ATGGTTCCATTTGCTTTGTCATCCAATTAGTGACTACTTTATATT |
| | | ATTCACTCCTCTTTATTACTATTTTCATGCGAGGTTGCCATGTAC |
| | | ATTATATTTGTAAGGATTGACGCTATTGAGCGTTTTTCTTCAATT |
| | | TTCTTTATTTTAGACATGGGTATGAAATGTGTGTTAGAGTTGGGT |
| | | TGAATGAGATATACGTTCAAGTGAAGTGGCATACCGTTGTCGAGT |
| | | AAGGATGACCTACCCATTCTTGAGACAAATGTTACATTTTAGTAT |
| | | CAGAGTAAAATGTGTACCTATAACTCAAATTCGATTGACATGTAT |
| | | CCATTCAACATAAAATTAAACCAGCCTGCACCTGCATCCACATTT |
| | | CAAGTATTTTCAAACCGTTCGGCTCCTATCCACCGGGTGTAACAA |
| | | GACGGATTCCGAATTTGGAAGATTTTGACTCAAATTCCCAATTTA |
| | | TATTGACCGTGACTAAATCAACTTTAACTTCTATAATTCTGATTA |
| | | AGCTCCCAATTTATATTCCCAACGGCACTACCTCCAAAATTTATA |
| | | GACTCTCATCCCCTTTTAAACCAACTTAGTAAACGTTTTTTTTTT |
| | | TAATTTTATGAAGTTAAGTTTTTACCTTGTTTTTAAAAAGAATCG |
| | | TTCATAAGATGCCATGCCAGAACATTAGCTACACGTTACACATAG |
| | | CATGCAGCCGCGGAGAATTGTTTTTCTTCGCCACTTGTCACTCCC |
| | | TTCAAACACCTAAGAGCTTCTCTCTCACAGCACACACATACAATC |
| | | ACATGCGTGCATGCATTATTACACGTGATCGCCATGCAAATCTCC |
| | | TTTATAGCCTATAAATTAACTCATCCGCTTCACTCTTTACTCAAA |
| | | CCAAAACTCATCAATACAAACAAGATTAAAAACATA |
| Signal | | |
| peptides | | |
| sig2 | 707 | ATGGCCAAGCTAGTTTTTTCCCTTTGTTTTCTGCTTTTCAGTGGC |
| | | TGCTGCTTCGCT |
| sig2 | 708 | MAKLVFSLCFLLFSGCCFA |
| sig10 | 709 | ATGGCTACTTCAAAGTTGAAAACCCAGAATGTGGTTGTATCTCTC |
| | | TCCCTAACCTTAACCTTGGTACTGGTGCTACTGACCAGCAAGGCA |
| | | AACTCA |
| sig10 | 710 | MATSKLKTQNVVVSLSLTLTLVLVLLTSKANS |
| sig11 | 711 | ATGATGAGAGCACGGTTCCCATTACTGTTGCTGGGACTTGTTTTC |
| | | CTGGCTTCAGTTTCTGTCTCA |
| sig11 | 712 | MMRARFPLLLLGLVFLASVSVS |
| sig12 | 713 | ATGATGAGAGCGCGGTTCCCATTACTGTTGCTGGGAGTTGTTTTC |
| | | CTGGCATCAGTTTCTGTCTCATTTGGC |
| sig12 | 714 | MMRARFPLLLLGVVFLASVSVSFG |
| coixss | 715 | ATGGCTACCAAGATATTTGCCCTCCTTGTGCTCCTTGCTCTTTCA |
| | | GCGAGCGCTACAACTGCG |
| coixss | 716 | MATKIFALLVLLALSASATTA |
| KDEL | 717 | AAGGATGAGCTT |
| KDEL | 616 | KDEL |
| Terminators | | |
| nosT | 718 | GATCGTTCAAACATTTGGCAATAAAGTTTCTTAAGATTGAATCCT |
| | | GTTGCCGGTCTTGCGATGATTATCATATAATTTCTGTTGAATTAC |
| | | GTTAAGCATGTAATAATTAACATGTAATGCATGACGTTATTTATG |
| | | AGATGGGTTTTTATGATTAGAGTCCCGCAATTATACATTTAATAC |
| | | GCGATAGAAAACAAAATATAGCGCGCAAACTAGGATAAATTATCG |
| | | CGCGCGGTGTCATCTATGTTACTAGATC |
| EU T | 719 | AAAGCAGAATGCTGAGCTAAAAGAAAGGCTTTTTCCATTTTCGAG |
| | | AGACAATGAGAAAAGAAGAAGAAGAAGAAGAAGAAGAAGAAGAAG |
| | | AAAAGAGTAAATAATAAAGCCCCACAGGAGGCGAAGTTCTTGTAG |
| | | CTCCATGTT |
| | | ATCTAAGTTATTGATATTGTTTGCCCTATATTTTATTTCTGTCAT |
| | | TGTGTATGTTTTGTTCAGTTTCGATCTCCTTGCAAAATGCAGAGA |
| | | TTATGAGATGAATAAACTAAGTTATATTATTATACGTGTTAATAT |
| | | TCTCCTCCTCTCTCTAGCTAGCCTTTTGTTTTCTCTTTTTCTTAT |
| | | TTGATTTTCTTTAAATCAATCCATTTTAGGAGAGGGCCAGGGAGT |
| | | GATCCAGCAAAACATGAAGATTAGAAGAAACTTCCCTCTTTTTTT |
| | | TCCTGAAAACAATTTAACGTCGAGATTTATCTCTTTTTGTAATGG |
| | | AATCATTTCTACAGTTATGAC |
| StUbi3T | 720 | CTGATTTTAATGTTTAGCAAATGTCTTATCAGTTTTCTCTTTTTG |
| | | TCGAACGGTAATTTAGAGTTTTTTTTGCTATATGGATTTTCGTTT |
| | | TTGATGTATGTGACAACCCTCGGGATTGTTGATTTATTTCAAAAC |
| | | TAAGAGTTTTTGTCTTATTGTTCTCGTCTATTTTGGAATATCAAT |
| | | CTTAGTTTTATATCTTTTCTAGTTCTCTACGTGTTAAATGTTCAA |
| | | CACACTAGCAATTTGGCCTGCCAGCGTATGGATTATGGAACTATC |
| | | AAGTGTGTGGGATCGATAAATATGCTTCTCAGGAATTTGAGATTT |
| | | TACAGTCTTTATGCTCATTGGGTTGAGTATAATATAGTAAAAAAA |
| | | TAGTAAATTTAAGCAATAATGTTAGGTGCTATGTGTCTGTCGAGA |
| | | CTATTGGCC |
| AtHSP T | 721 | ATATGAAGATGAAGATGAAATATTTGGTGTGTCAAATAAAAAGCT |
| | | TGTGTGCTTAAGTTTGTGTTTTTTTCTTGGCTTGTTGTGTTATGA |
| | | ATTTGTGGCTTTTTCTAATATCAAATGAATGTAAGATCTCATTAT |
| | | AATGAATAAACAAATGTTTCTATAATCCATTGTGAATGTTTTGTT |
| | | GGATCTCTTCTGCAGCATATAACTACTGTATGTGCTATGGTATGG |
| | | ACTATGGAATATGATTAAAGATAA |
| AtUbi10T | 722 | ATCTCGTCTCTGTTATGCTTAAGAAGTTCAATGTTTCGTTTCATG |
| | | TAAAACTTTGGTGGTTTGTGTTTTGGGGCCTTGTATAATCCCTGA |
| | | TGAATAAGTGTTCTACTATGTTTCCGTTCCTGTTATCTCTTTCTT |
| | | TCTAATGACAAGTCGAACTTCTTCTTTATCATCGCTTCGTTTTTA |
| | | TTATCTGTGCTTCTTTTGTTTAATACGCCTGCAAAGTGACTCGAC |
| | | TCTGTTTAGTGCAGTTCTGCGAAACTTGTAAATAGTCCAATTGTT |
| | | GGCCTCTAGTAATAGATGTAGCGAAAGTGTTGAGCTGTTGGGTTC |
| | | TAAGGATGGCTTGAACATGTTAATCTTTTAGGTTCTGAGTATGAT |
| | | GAACATTCGTTGTTGC |
| Rb7T | 723 | TAAAATGCGTCAATCTCTTTGTTCTTCCATATTCATATGTCAAAA |
| | | TCTATCAAAATTCTTATATATCTTTTTCGAATTTGAAGTGAAATT |
| | | TCGATAATTTAAAATTAAATAGAACATATCATTATTTAGGTATCA |
| | | TATTGATTTTTATACTTAATTACTAAATTTGGTTAACTTTGAAAG |
| | | TGTACATCAACGAAAAATTAGTCAAACGACTAAAATAAATAAATA |
| | | TCATGTGTTATTAAGAAAATTCTCCTATAAGAATATTTTAATAGA |
| | | TCATATGTTTGTAAAAAAAATTAATTTTTACTAACACATATATTT |
| | | ACTTATCAAAAATTTGACAAAGTAAGATTAAAATAATATTCATCT |
| | | AACAAAAAAAAAACCAGAAAATGCTGAAAACCCGGCAAAACCGAA |
| | | CCAATCCAAACCGATATAGTTGGTTTGGTTTGATTTTGATATAAA |
| | | CC |
| TM6T | 724 | GAACCAACTCGGTCCATTTGCACCCCTAATCATAATAGCTTTAAT |
| | | ATTTCAAGATATTATTAAGTTAACGTTGTCAATATCCTGGAAATT |
| | | TTGCAAAATGAATCAAGCCTATATGGCTGTAATATGAATTTAAAA |
| | | GCAGCTCGATGTGGTGGTAATATGTAATTTACTTGATTCTAAAAA |
| | | AATATCCCAAGTATTAATAATTTCTGCTAGGAAGAAGGTTAGCTA |
| | | CGATTTACAGCAAAGCCAGAATACAAAGAACCATAAAGTGATTGA |
| | | AGCTCGAAATATACGAAGGAACAAATATTTTTAAAAAAATACGCA |
| | | ATGACTTGGAACAAAAGAAAGTGATATATTTTTTGTTCTTAAACA |
| | | AGCATCCCCTCTAAAGAATGGCAGTTTTCCTTTGCATGTAACTAT |
| | | TATGCTCCCTTCGTTACAAAAATTTTGGACTACTATTGGGAACTT |
| | | CTTCTGAAAATAGTGACATCCTAGGTTCAATCAAATTTTACTCGC |
| | | ATATTGTAGACTTTATCCTTTTGTAATTGTTGCAAATTTCTTATA |
| | | AAATTGATTATCTATATTTTAATCAAACATATATATACACTTCCA |
| | | AATAATAAAATATAATGACAACAAAACAATCAAGCACAAAAAATG |
| | | CCTATAACAAATAAAAATTACAACATACTTTTACCCTGATTCAAA |
| | | TCTTCAAACACTATGCCAGACACCATAATCCTTCTGGATATAGGA |
| | | TAAAAATTTAAAGTGATTTTTTACCAATTACTATTTCATAAATTG |
| | | TTCAAATACAAAATATGATATTTTAATTATTCCCAACTTTTTGAG |
| | | CCTCCTATAACTAATCAATATAAAAAAATAATTTATCGATTAAGA |
| | | CTAAAGCAAAAAATATTACCGATTTGAGTTACAATAAAAAGTTTT |
| | | ATATCACGTTATGGTATTGTGAATTACTCTAACTTCCTAGTTCTT |
| | | GGGTTCTAGCTTTTCTTGGCTCTCTGAATCTTCAAAACCTATATT |
| | | TGATAAAGCCATAACATACACTAATGCTCCCATGCAAAGTGCTTC |
| | | TAAAACTCCTTAACTTGGTCTACGGTAAAATTTCTTCTAAAACAA |
| | | AAGCGACTATCAACTTCTAATCGTTGAACAAATAATTCATCTCCA |
| | | ATAAAGGATTTTAACAATAAATATGAAATAAGAAGTCTATTTCTA |
| | | GTTAAATAACCAACAATATCCCAAACATTTATGAAATCAATATAT |
| | | GACTGCATTACAATTTGATCCCAAAATGCAAAAATAAAATTGCAT |
| | | CTCTATTATAGAGTAAAAATAATGCATCATCAATTACTAACCGAT |
| | | TTTACTAACACGAGAATCTAATTCTCTTCCACAAAGTAAAACTCA |
| | | ATGTCACCGTCAATTATTTAAGAATTTGAATTATATTCCAACAAC |
| | | TGAGTAAGAAACTATATAATTGTGGGGGGAGGGGGGGCCAACCCT |
| | | AAAAGTTTACTTCTCATAAAAGGCTATTAGAAAGGAAAGGATACA |
| | | TAAAAAGAAGAGCAAAGAGAGATCGGAGAAGAGAGAAAAAGTATA |
| | | TGAATTTATTAGAAGTACTTTTACTTATTAGAGGTAAGAGAGTTC |
| | | TAGACTGATTTGGATACCATATTAGAGTTATTACCGATATAAAAA |
| | | TCCTTGGTTATGTTAATTAAATTTCTAAATATTA |
| arcT | 725 | AATAAATAAAATGGGAGCAATAAATAAAATGGGAGCTCATATATT |
| | | TACACCATTTACACTGTCTATTATTCACCATGCCAATTATTACTT |
| | | CATAATTTTAAAATTATGTCATTTTTAAAAATTGCTTAATGATGG |
| | | AAAGGATTATTATAAGTTAAAAGTATAACATAGATAAACTAACCA |
| | | CAAAACAAATCAATATAAACTAACTTACTCTCCCATCTAATTTTT |
| | | ATTTAAATTTCTTTACACTTCTCTTCC |
| | | ATTTCTATTTCTACAACATTATTTAACATTTTTATTGTATTTTTC |
| | | TTACTTTCTAACTCTATTCATTTCAAAAATCAATATATGTTTATC |
| | | ACCACCTCTCTAAAAAAAACTTTACAATCATTGGTCCAGAAAAGT |
| | | TAAATCACGAGATGGTCATTTTAGCATTAAAACAACGATTCTTGT |
| | | ATCACTATTTTTCAGCATGTAGTCCATTCTCTTCAAACAAAGACA |
| | | GCGGCTATATAATCGTTGTGTTATATTCAGTCTAAAACAATTGTT |
| | | ATGGTAAAAGTCGTCATTTTACGCCTTTTTAAAAGATATAAAATG |
| | | ACAGTTATGGTTAAAAGTCATCATGTTAGATCCTCCTTAAAGATA |
| | | TAAAATGACAGTTTTGGATAAAAAGTGGTCATTTTATACGCTCTT |
| | | GAAAGATATAAAACGACGGTTATGGTAAAAGCTGCCATTTTAAAT |
| | | GAAATATTTTTGTTTTAGTTCATTTTGTTTAATGCTAATCCCATT |
| | | TAAATTGACTTGTACAATTAAAACTCACCCACCCAGATACAATAT |
| | | AAACTAACTTACTCTCACAGCTAAGTTTTATTTAAATTTCTTTAC |
| | | ACTTCTTTTCCATTTCTATTTCTATGACATTAACTAACATTTTTC |
| | | TCGTAATTTTTTTTCTTATTTTCTAACTCTATCCATTTCAAATCG |
| | | ATATATGTTTATCACCACCACTTTAAAAAGAAAATTTACAATTTC |
| | | TCGTGCAAAAAAGCTAAATCATGACCGTCATTTTAGCATTAAAAC |
| | | AACGATTCTTGTATCGTTGTTTTTCAGCATGTAGTCCATTCTTTT |
| | | CAAGCAAAGACAACAGCTATATAATCATCGTGTTATATTCAGTCT |
| | | AAAACAACAGTAATGATAAAAGTCATCATTTTAGGCCTTTCTGAA |
| | | ATATATAGAACGACATTCATGGTAAAAAATCGTCATTTTAGATCC |
| 5′UTRs | | |
| soybean | 726 | GAATTCTCTAAAAGAGATCTTTTTCTGCTCTTTGAAGAAAGAAGG |
| glutamine | | GTCTTTGCTTGATTTTGGAG |
| synthase | | |
| OVAL | 727 | ACATACAGCTAGAAAGCTGTATTGCCTTTAGCACTCAAGCTCAAA |
| | | AGACAACTCAGAGTTCACC |
| LG | 728 | CCCGAGCCCGCTGTCTCAGCCCTCCACTCCCTGCAGAGCTCAGAA |
| | | GCGTGACCCCAGCTGCAGCC |
| arcUTR | 729 | TGAATGCATGATC |
| Monomer | | |
| Ubimonomer | 730 | ATGCAGATTTTCGTGAAGACCTTAACGGGGAAGACGATCACCCTA |
| | | GAGGTTGAGTCTTCCGACACCATCGACAATGTCAAAGCCAAGATC |
| | | CAGGACAAGGAAGGGATACCCCCAGACCAGCAGCGTTTGATTTTC |
| | | GCCGGAAAGCAGCTTGAGGATGGTCGTACTCTTGCCGACTACAAC |
| | | ATCCAGAAGGAGTCAACTCTCCATCTCGTGCTCCGTCTCCGTGGT |
| | | GGTGGTTCC |
| Ubimonomer | 731 | MQIFVKTLTGKTITLEVESSDTIDNVKAKIQDKEGIPPDQQRLIF |
| | | AGKQLEDGRTLADYNIQKESTLHLVLRLRGGGS |

Selection and reporter gene cassette components: Promoter

| | | |
|---|---|---|
| CaMV35S | 785 | TCAGCGTGTCCTCTCCAAATGAAATGAACTTCCTTATATAGAGGA |
| | | AGGTCTTGCGAAGGATAGTGGGATTGTGCGTCATCCCTTACGTCA |
| | | GTGGAGATATCACATCAATCCACTTGCTTTGAAGACGTGGTTGGA |
| | | ACGTCTTCTTTTTCCACGATGCTCCTCGTGGGTGGGGGTCCATCT |
| | | TTGGGACCACTGTCGGCAGAGGCATCTTGAACGATAGCCTTTCCT |
| | | TTATCGCAATGATGGCATTTGTAGGTGCCACCTTCCTTTTCTACT |
| | | GTCCTTTTGATGAAGTGACAGATAGCTGGGCAATGGAATCCGAGG |
| | | AGGTTTCCCGATATTACCCTTTGTTGAAAAGTCTCAATAGCCCTT |
| | | TGGTCTTCTGAGACTGTATCTTTGATATTCTTGGAGTAGACGAGA |
| | | GTGTCGTGCTCCACCATGTTATCACATCAATCCACTTGCTTTGAA |
| | | GACGTGGTTGGAACGTCTTCTTTTTCCACGATGCTCCTCGTGGGT |
| | | GGGGGTCCATCTTTGGGACCACTGTCGGCAGAGGCATCTTGAACG |
| | | ATAGCCTTTCCTTTATCGCAATGATGGCATTTGTAGGTGCCACCT |
| | | TCCTTTTCTACTGTCCTTTTGATGAAGTGACAGATAGCTGGGCAA |
| | | TGGAATCCGAGGAGGTTTCCCGATATTACCCTTTGTTGAAAAGTC |
| | | TCA |
| GmU3 | 786 | GGGCCCAATATAACAACGACGTCGTAACAGATAAAGCGAAGCTTG |
| | | AAGGTGCATGTGACTCCGTCAAGATTACGAAACCGCCAACTACCA |
| | | CGCAAATTGCAATTCTCAATTTCCTAGAAGGACTCTCCGAAAATG |
| | | CATCCAATACCAAATATTACCCGTGTCATAGGCACCAAGTGACAC |
| | | CATACATGAACACGCGTCACAATATGACTGGAGAAGGGTTCCACA |
| | | CCTTATGCTATAAAACGCCCCACACCCCTCCTCCTTCCTTCGCAG |
| | | TTCAATTCCAATATATTCCATTCTCTCTGTGTATTTCCCTACCTC |
| | | TCCCTTCAAGGTTAGTCGATTTCTTCTGTTTTTCTTCTTCGTTCT |
| | | TTCCATGAATTGTGTATGTTCTTTGATCAATACGATGTTGATTTG |
| | | ATTGTGTTTTGTTTGGTTTCATCGATCTTCAATTTTCATAATCAG |
| | | ATTCAGCTTTTATTATCTTTACAACAACGTCCTTAATTTGATGAT |
| | | TCTTTAATCGTAGATTTGCTCTAATTAGAGCTTTTTCATGTCAGA |
| | | TCCCTTTACAACAAGCCTTAATTGTTGATTCATTAATCGTAGATT |
| | | AGGGCTTTTTTCATTGATTACTTCAGATCCGTTAAACGTAACCAT |
| | | AGATCAGGGCTTTTTCATGAATTACTTCAGATCCGTTAAACAACA |
| | | GCCTTATTTTTTATACTTCTGTGGTTTTTCAAGAAATTGTTCAGA |
| | | TCCGTTGACAAAAAGCCTTATTCGTTGATTCTATA |
| | | TCGTTTTTCGAGAGATATTGCTCAGATCTGTTAGCAACTGCCTTG |
| | | TTTGTTGATTCTATTGCCGTGGATTAGGGTTTTTTTTCACGAGAT |
| | | TGCTTCAGATCCGTACTTAAGATTACGTAATGGATTTTGATTCTG |
| | | ATTTATCTGTGATTGTTGACTCGACAG |
| StUbi3 | 787 | GGCCAAAGCACATAGTTATCGATTTAAATTTCATCGAAGAGATTA |
| | | ATATCGAATAATCATATACATACTTTAAATACATAACAAATTTTA |
| | | AATACATATATCTGGTATATAATTAATTTTTTAAAGTCATGAAGT |
| | | ATGTATCAAATACAAATATGGAAAAAATTAACTATTCATAATTTA |
| | | AAAAATAGAAAAGATACATCTAGTGAAATTAGGTGCATGTATCAA |
| | | ATACATTAGGAAAAGGGCATATATCTTGATCTAGATAATTAACGA |
| | | TTTTGATTTATGTATAATTTCCAAATGAAGGTTTATATCTACTTC |
| | | AGAAATAACAATATACTTTTATCAGAACATTCAACAAAGCAACAA |
| | | CCAACTAGAGTGAAAAATACACATTGTTCTCTAGACATACAAAAT |
| | | TGAGAAAAGAATCTCAAAATTTAGAGAAACAAATCTGAATTTCTA |
| | | GAAGAAAAAAATAATTATGCACTTTGCTATTGCTCGAAAAATAAA |
| | | TGAAAGAAATTAGACTTTTTTAAAAGATGTTAGACTAGATATACT |
| | | CAAAAGCTATTAAAGGAGTAATATTCTTCTTACATTAAGTATTTT |
| | | AGTTACAGTCCTGTAATTAAAGACACATTTTAGATTGTATCTAAA |
| | | CTTAAATGTATCTAGAATACATATATTTGATTGCATCATATCCAT |
| | | GTATCCGACACACCAATTCTCATAAAAAACGTAATATCCTAAACT |
| | | AATTTATCCTTCAAGTCAACCTAAGCCCAATATACATTTTCATCT |
| | | CTAAAGGCCCAAGTGGCACAAAATGTCAGGCCCAATTACGAAGAA |
| | | AAGGGCTTGTAAAACCCTAATAAAGTGGCACTGGCAGAGCTTACA |
| | | CTCTCATTCCATCAACAAAGAAACCCTAAAAGCCGCAGCGCCACT |
| | | GATTTCTCTCCTCCAGGCGAAG |

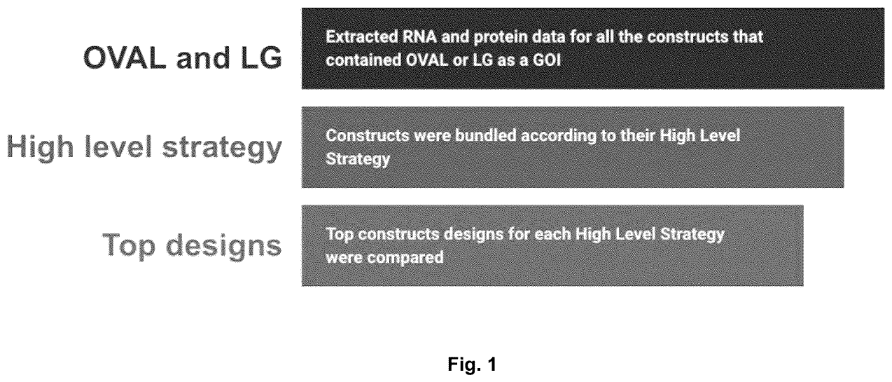

Data was bundled as shown in FIG. 1.

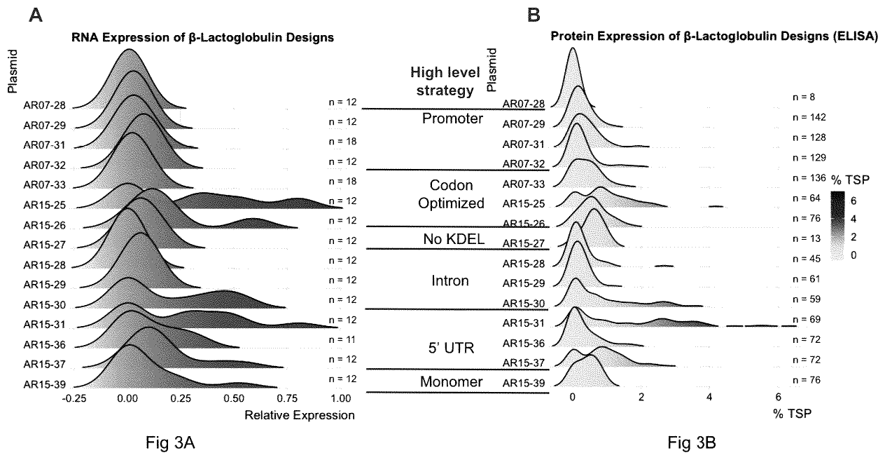

In the case of LG, a similar effect was seen in which RNA expression was lower (highest 0.14× glycinin) than expected under different promoters (FIG. 3A), however, unlike OVAL, protein accumulation was significant. GmSeed12: coixx (AR07-31), GmSeed12:sig12 (AR07-32), and PvPhas (AR07-33) showed the best results, producing the highest number of seeds with >100 TSP. Some seeds expressing transgene under control of the promoter GmSeed12: coixx (AR07-31) contained up to 2.53% TSP LG (FIG. 3, Table 12).

TABLE 12
Summary of RNA and protein quantification for exemplary LG designs

| | | | | | | | # of | # of |
|---|---|---|---|---|---|---|---|---|
| | | | | # of all | # of all | # of | seeds | seeds |
| | | | | plants | seeds | seeds | with | with |
| | | | | analyzed | analyzed | below | 0.063- | over |
| Construct | | Highest | Max | for | for | detection | 1% | 1% |
| ID | Details | RNA level | % TSP | ELISA | ELISA | level | TSP | TSP |
| AR07-28 | BnNap:sig11:OLG1:KDEL:nos | 0.03 | 0.00 | 1 | 8 | 8 | 0 | 0 |
| AR07-29 | GmSeed2:sig2:OLG1:KDEL:nos | 0.08 | 1.23 | 18 | 142 | 49 | 90 | 3 |
| AR07-31 | GmSeed12:coixss:OLG1:KDEL:nos | 0.12 | 2.53 | 15 | 128 | 32 | 88 | 8 |
| AR07-32 | GmSeed12:sig12:OLG1:KDEL:nos | 0.14 | 2.05 | 17 | 129 | 53 | 68 | 8 |
| AR07-33 | PvPhas:arcUTR:sig10:OLG1:KDEL:arc T | 0.07 | 2.38 | 17 | 136 | 47 | 79 | 10 |
| AR15-25 | GmSeed2:sig2:OLG2:KDEL:EUT:Rb7T | 0.78 | 4.18 | 8 | 64 | 15 | 25 | 24 |
| AR15-26 | GmSeed2:sig2:OLG3:KDEL:EUT:Rb7T | 0.60 | 1.79 | 10 | 76 | 10 | 53 | 13 |
| AR15-27 | GmSeed2:sig2:OLG4:KDEL:EUT:Rb7T | 0.14 | 1.05 | 2 | 13 | 1 | 11 | 1 |
| AR15-28 | GmSeed2:sig2:OLG2:EUT:Rb7T | All GOI | 2.66 | 8 | 45 | 20 | 23 | 2 |
| | | silenced* | | | | | | |
| AR15-29 | GmSeed2 (intron | 0.11 | 1.21 | 10 | 61 | 27 | 33 | 1 |
| | 1):sig2:OLG2:KDEL:EUT:Rb7T | | | | | | | |
| AR15-30 | GmSeed2:sig2:OLG2 (intron | 0.55 | 3.58 | 9 | 59 | 16 | 25 | 18 |
| | 1):KDEL:EUT:Rb7T | | | | | | | |
| AR15-31 | GmSeed2:sig2:OLG2 (intron | 0.80 | 6.33 | 9 | 69 | 17 | 17 | 35 |
| | 2):KDEL:EUT:Rb7T | | | | | | | |
| AR15-36 | GmSeed2:1gUTR:sig2:OLG2:KDEL: | 0.32 | 1.82 | 9 | 72 | 41 | 26 | 5 |
| | EUT:Rb7T | | | | | | | |
| AR15-37 | GmSeed2:glnB1UTR:sig2:OLG2: | 0.55 | 2.81 | 9 | 72 | 20 | 28 | 24 |
| | KDEL:EUT:Rb7T | | | | | | | |
| AR15-39 | GmSeed2:Ubimonomer:sig2:OLG2: | 0.52 | 0.92 | 9 | 76 | 22 | 54 | 0 |
| | KDEL:EUT:Rb7T | | | | | | | |

*Three plants were analyzed for RNA levels for constructs AR15-28. No detectable transgene expression was observed.

### Example 3: Use of Codon Optimization to Enhance Stability of RNA and Increase Protein Levels

Codon usage bias can influence protein levels when recombinant proteins are expressed in host cells which do not natively express the protein. Initially, OVAL and LG were codon optimized (OOVAL1 and OLG1, respectively) using soybean's codon usage bias. When the different promoters were tested, the sequences were further optimized by analyzing the structure of the codon-optimized RNAs in silico, using the RNAfold program (rna.tbi.univie.ac.at//cgi-bin/RNAWebSuite/RNAfold.cgi?PAGE=3&ID=zsy5friDrE\). Different parameters were analyzed, including minimum free energy structures (MFE), base pair probabilities and energy mountain plot. In addition, the location of 5′ and start codon within MFE structure were determined, and RNA structure analysis was focused within that region. By analyzing these different thermodynamic parameters, RNA stem loops were identified which could lead to a stable RNA structure. Specifically, the optimal length of the stem loop was about 4-8 bp, containing a tetraloop UUCG (SEQ ID NO: 615). Codon optimized sequences predicted to present pseudo-knots, such as large loops with no secondary structure of their own and loops of less than 4 and more than 8 bp, were predicted to be unstable. Based on several iterations of this analysis, it was predicted that version 2-4 of OVAL and LG would have the desired structural characteristics that are known to stabilize RNA. Accordingly, these codon optimized versions were chosen for soybean transformation.

In the case of OVAL, RNA expression was slightly improved in the codon optimized versions of OOVAL2 (0.11× glycinin) and OOVAL3 (0.08× glycinin). Additionally, the use of the double terminator EUT:Rb7T when compared to the control that is driven by the same promoter (AR07-23) that had an expression of 0.03× glycinin (FIG. 2A and Table 10). Surprisingly, the slight improvement in RNA expression led to a significant increase in protein accumulation with OOVAL 2 and OOVAL3 producing seeds that contained up to 2.74 and 1.43% TSP (FIG. 2B and Table 10). There was limited RNA expression improvement in the plants expressing OOVAL4 and their protein analysis was not pursued.

In the case of LG, there was a significant improvement with the codon optimized versions of OLG2 (0.78× glycinin) and the use of the double terminator EUT:Rb7T when compared to the control that is driven by the same promoter (AR07-29), which had an expression level of 0.08× glycinin (FIG. 3A and Table 12). The increase in RNA expression translated into an increase in protein accumulation, with some seeds producing up to 4.18 and 1.79% TSP for OLG2 and OLG3, respectively.

### Example 4: Addition or Removal of KDEL to Enhance Stability of RNA and Increase Protein Levels

Proteins can be targeted to specific cellular compartments such as the endoplasmic reticulum (ER), vacuole, cytoplasm, protein storage vacuole (PSV) and apoplast, using specific peptide tags. The localization of recombinant proteins to different organelles offers a valuable strategy for recombinant protein production since a wide range of proteases are found in each of these organelles. Unlike the other organelles, the ER is a protective environment due to the low abundance of proteases. Accordingly, the ER is an optimal location to target recombinant proteins due to the abundance of chaperones that help in the folding thereof. Recombinant proteins can be targeted to the ER by the addition of the ER peptide tag KDEL (Lys-Asp-Glu-Leu (SEQ ID NO: 616) or AAGGAUGAGCUU (SEQ ID NO: 629) for RNA) at the N-terminus. Surprisingly, while analyzing the RNA expression levels, it was observed that the addition of AAGGAUGAGCUU (SEQ ID NO: 629) enhances the RNA stability, independent of its effects on protein targeting See Table 13, positive or negative.

TABLE 13
RNA expression levels with or without KDEL. Line averages
of plant max RNA expression (out of 4 seeds per plant),
excluding plants with no detectable transgene RNA.

| Transgene | KDEL status | RNA levels |
|---|---|---|
| OVAL | +KDEL | 0.044 |
| | −KDEL | 0.120 |
| LG | +KDEL | 0.765 |
| | −KDEL | <0.001 |

### Example 5: Use of Introns to Enhance Stability of RNA and Increase Protein Levels

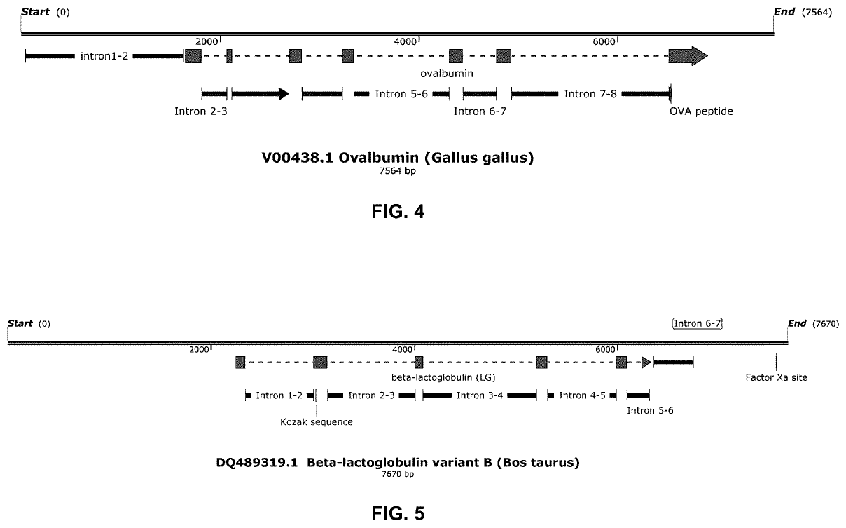

In this study, the effects of two introns were examined: Intron 1 and 2 from the Elongation Factor 1A (eF1A) gene from Glycine max or Arabidopsis thaliana, respectively (Table 14). Additionally, their effect on gene expression was analyzed by placing them in different locations within the DNA construct—either in the 5′UTR or within the coding sequence. For the designs with the intron at the 5′ UTR, intron 1 was used and it was placed in between the GmSeed2 promoter and the start of either OVAL or LG (AR15-20 and AR15-29, respectively). In the designs wherein the intron was located within the coding region, OVAL's native intron 2-3 (FIG. 4) or LG's native intron 1-2 was replaced with either intron 1 or 2 (FIG. 5) (OVAL: AR15-21 and AR15-22 and LG: AR15-30 and AR15-31). The data provided herein shows that the use of intron 1 at the 5′UTR had a negative effect on both RNA and protein levels, for both OVAL and LG. When compared to the DNA construct designs lacking the intron (AR15-16 and AR15-25, OVAL and LG, respectively), the RNA expression dropped from 0.11 to 0.03× glycinin for OVAL and from 0.78 to 0.11× glycinin for LG, leading to low protein expression for LG (OVAL plants were not analyzed due to low RNA expression).

When the intron was placed within the coding sequence, a moderate change was observed at the RNA level, but a significant improvement was obtained at the protein level. Interestingly, intron 1 and 2 had different effects on OVAL and LG. In the case of OVAL, intron 1 (AR15-21) had a greater effect leading to 64% of the seeds expressing at >1% TSP (control 8.7% of the seeds had >1% TSP (AR15-16)). Also, there was a significant increase in the Max % TSP from 2.74 (AR15-16) to 6.64% TSP (AR15-21). In the case of LG, intron 2 (AR15-31) had a pronounced effect, leading to 50% of the seeds expressing at >1% TSP (control 37% of the seeds had >1% TSP (AR15-25). Also, there was a noteworthy increase in the Max % TSP from 4.18 (AR15-25) to 6.33% TSP (AR15-31) in the designs that contained intron 2.

TABLE 14
Exemplary introns utilized

| | Intron/ | | | | | |
|---|---|---|---|---|---|---|
| Intron | intron | | Length | IMEter | | |
| name | fragments | Glyma. Genebank | (bp) | score V2.1 | Percentile | Reference |
| Intron 1 | Intron 1 | X56856.1 | 770 | 16.03 | 98 | DOI:10.1371/journal. |
| | from | | | | | pone.0166074 |
| | Elongation | | | | | |
| | factor 1A | | | | | |
| | (Glycine | | | | | |
| | max) | | | | | |
| Intron 2 | Intron 1 | X16430.1 | 99 | 2.04 | 49 | — |
| | from | | | | | |
| | Elongation | | | | | |
| | factor 1A | | | | | |
| | (Arabidopsis | | | | | |
| | thaliana) | | | | | |

### Example 6: Materials and Methods Utilized in the Present Examples

### Binary Vector Design

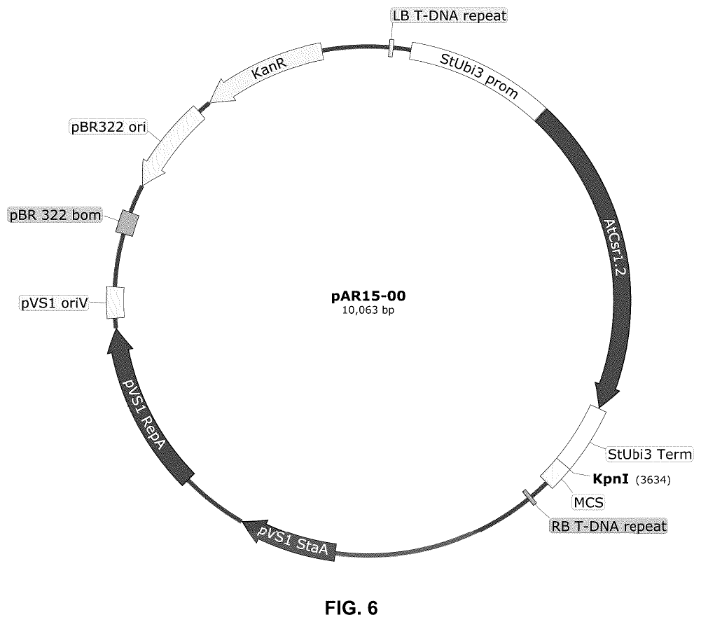

The binary pCAMBIA3300 (Creative Biogene, VET1372) vector provides features such as high copy number in E. coli for high DNA yields, the pVS1 replicon for stable expression in Agrobacterium, a multiple cloning site to allow plasmid modifications and a kanamycin bacterial selection that permits the vector to move DNA from bacteria to the desired plant host. Therefore, this vector was customized to include a selectable marker suitable for soybean transformation and selection. In order to modify the vector, pCAMBIA3300 was digested with HindIII and AseI allowing the release of the vector backbone (LB T-DNA repeat_KanR_pBR322 ori_pBR322 bom_pVS1 oriV_pVsl repA_pVS1 StaA_RB T-DNA repeat). The 6598 bp vector backbone was gel extracted and a synthesized multiple cloning site (MCS) was ligated via In-Fusion cloning (In-Fusion® HD Cloning System CE) to allow modular vector modifications and to create the vector backbone. A cassette containing the Arabidopsis thaliana Csr1.2 gene for acetolactate synthase was added to the vector backbone to be used as a marker for herbicide selection of transgenic plants. In order to build this cassette, the regulatory sequences from Solanum tuberosum ubiquitin/ribosomal fusion protein promoter (StUbi3 prom; −1 to −922 bp) and terminator (StUbi3 term; 414 bp) (accession no. L22576.1) were fused to the mutant (S653N) acetolactate synthase gene (Csr1.2; accession no. X51514.1) (Sathasivan et al 1990, Ding et al 2006) to generate imazapyr-resistant traits in soybean plants. The selectable marker cassette was introduced into the digested (EcoRI) modified vector backbone via In-Fusion cloning to form vector pAR15-00 (FIG. 6).

Vector pAR07-00 was assembled to include two cassettes, comprising an antibiotic selection and a reporter gene cassette. The antibiotic selection cassette contained the E. coli aadA gene for aminoglycoside adenylyltransferase (aadA; accession no. AB188259), which confers resistance to spectinomycin for the selection of transgenic plants. The regulatory elements in this cassette included the 35S promoter (enhanced) (35 s prom; 678 bp) from Cauliflower mosaic virus to promote expression of aadA gene, the 5-enol-pyruvylshikimate-3-phosphate synthase (EPSPS) signal peptide (EPSPSss; 216 bp) (accession no. KJ787649.1) from Petunia hybrida for localization of aadA into the chloroplast, and the 35S poly(A) signal (35 s Term; 191 bp) from Cauliflower mosaic virus for aadA transcription stabilization. On the other hand, the selection marker cassette carried the mCherry fluorescent reporter gene from Discosoma sp. for rapid phenotypic selection of transgenic plants. The regulatory components for this cassette included the Glycine max Ubiquitin promoter (GmU3 prom; 917 bp) (accession no. EU310508) to promote the expression of mCherry, and the ribulose-1,5-bisphospate carboxylase small subunit termination sequence from Pisum sativum (RbcS-E9; 297 bp) (accession no. X00806.1) for mCherry transcription stabilization. Both cassettes were introduced into the digested vector backbone via In-Fusion cloning to form vector pAR07-00.

Vector pAR15-00 was constructed containing the Arabidopsis thaliana Csr1.2 gene for acetolactate synthase to be used as a marker for herbicide selection of transgenic plants. In order to build this cassette, the regulatory sequences from Solanum tuberosum ubiquitin/ribosomal fusion protein promoter (StUbi3 prom; −1 to −922 bp) and terminator (StUbi3 term; 414 bp) (accession no. L22576.1) were fused to the mutant (S653N) acetolactate synthase gene (Csr1.2; accession no. X51514.1) (Sathasivan et al 1990, Ding et al 2006) to generate imazapyr-resistant traits in soybean plants. The selectable marker cassette was introduced into the digested (EcoRI) modified vector backbone via In-Fusion cloning to form vector pAR15-00.

### DNA Constructs

The components of each construct, see Table 15, were PCR amplified from either genomic DNA or synthesized fragments and assembled into a digested (KpnI) AR15-00 cloning vector using In-Fusion ligation (In-Fusion® HD Cloning System CE). Binary vectors were then transformed into Agrobacterium strain AGL1. Single colonies were verified for the presence of the vector via PCR using gene specific primers.

TABLE 15
Exemplary DNA constructs encoding ovalbumin or B-Lactoglobulin. Transcriptional
Regulation (TR), Protein Stability (PS),

| Construct ID | SEQ ID NO | Details | Protein | Category |
|---|---|---|---|---|
| AR07-22 | 752 | bnNap:sig11:OOVALI:KDEL:nos | Ovalbumin | TR/Promoter |
| AR07-23 | 753 | gmSeed2:sig2:OOVAL1:KDEL:nos | Ovalbumin | TR/Promoter |
| AR07-25 | 754 | gmSeed12:coixss:OOVAL1:KDEL:nos | Ovalbumin | TR/Promoter |
| AR07-26 | 755 | gmSeed12:sig12:OOVAL1:KDEL:nos | Ovalbumin | TR/Promoter |
| AR07-27 | 756 | pvPhas:arcUTR:sig10:OOVAL1:KDEL:arcT | Ovalbumin | TR/Promoter |
| AR15-16 | 757 | GmSeed2:sig2:OOVAL2:KDEL:EUT:Rb7T | Ovalbumin | TR/Codon |
| | | | | Optimized |
| AR15-17 | 758 | GmSeed2:sig2:OOVAL3:KDEL:EUT:Rb7T | Ovalbumin | TR/Codon |
| | | | | Optimized |
| AR15-18 | 759 | GmSeed2:sig2:OOVAL4:KDEL:EUT:Rb7T | Ovalbumin | TR/Codon |
| | | | | Optimized |
| AR15-19 | 760 | GmSeed2:sig2:OOVAL2:EUT: Rb7T | Ovalbumin | PS/No KDEL |
| AR15-20 | 761 | GmSeed2 (intron 1):sig2:OOVAL2:KDEL:EUT:Rb7T | Ovalbumin | TR/Intron |
| AR15-21 | 762 | GmSeed2:sig2:OOVAL2 (intron 1):KDEL:EUT:Rb7T | Ovalbumin | TR/Intron |
| AR15-22 | 763 | GmSeed2:sig2:OOVAL2 (intron 2):KDEL:EUT:Rb7T | Ovalbumin | TR/Intron |
| AR15-23 | 764 | GmSeed2:ovalUTR:sig2:OOVAL2:KDEL:EUT:Rb7T | Ovalbumin | TR/5′UTR |
| AR15-24 | 765 | GmSeed2:glnB1UTR:sig2:OOVAL2:KDEL:EUT:Rb | Ovalbumin | TR/5′UTR |
| | | 7T | | |
| AR15-38 | 766 | GmSeed2: Ubimonomer:sig2:OOVAL2:KDEL:EUT:R | Ovalbumin | TR/Monomer |
| | | b7T | | |
| AR07-28 | 767 | bnNap:sig11:OLG1:KDEL:nos | Betalactogobulin | TR/Promoter |
| AR07-29 | 768 | gm Seed2:sig2:OLG1:KDEL:nos | Betalactogobulin | TR/Promoter |
| AR07-31 | 769 | gm Seed12:coixss:OLG1:KDEL:nos | Betalactogobulin | TR/Promoter |
| AR07-32 | 770 | gmSeed12:sig12:OLG1:KDEL:nos | Betalactogobulin | TR/Promoter |
| AR07-33 | 771 | pvPhas:arcUTR:sig10:OLG1:KDEL:arcT | Betalactogobulin | TR/Promoter |
| AR15-25 | 772 | GmSeed2:sig2:OLG2:KDEL:EUT: Rb7T | Betalactogobulin | TR/Codon |
| | | | | Optimized |
| AR15-26 | 773 | GmSeed2:sig2:OLG3:KDEL:EUT: Rb7T | Betalactogobulin | TR/Codon |
| | | | | Optimized |
| AR15-27 | 774 | GmSeed2:sig2:OLG4:KDEL:EUT: Rb7T | Betalactogobulin | TR/Codon |
| | | | | Optimized |
| AR15-28 | 775 | GmSeed2:sig2:OLG2:EUT:Rb7T | Betalactogobulin | PS/No KDEL |
| AR15-29 | 776 | GmSeed2 (intron 1):sig2:OLG2:KDEL:EUT:Rb7T | Betalactogobulin | TR/Intron |
| AR15-30 | 777 | GmSeed2:sig2:OLG2 (intron 1):KDEL:EUT:Rb7T | Betalactogobulin | TR/Intron |
| AR15-31 | 778 | GmSeed2:sig2:OLG2 (intron 2):KDEL:EUT:Rb7T | Betalactogobulin | TR/Intron |
| AR15-36 | 779 | GmSeed2:1gUTR:sig2:OLG2:KDEL:EUT:Rb7T | Betalactogobulin | TR/5′UTR |
| AR15-37 | 780 | GmSeed2:glnB1UTR:sig2:OLG2:KDEL:EUT:Rb7T | Betalactogobulin | TR/5′UTR |
| AR15-39 | 781 | GmSeed2: Ubimonomer:sig2:OLG2:KDEL:EUT:Rb7 | Betalactogobulin | TR/Monomer |
| AR15-00 | 782 | | — | Plasmid |
| — | 783 | StUbi3:AtCsr1.2:StUbi3T | Acetolactate | Selection |
| | | | synthase | marker in |
| | | | | AR15-00 |
| AR07-00 | 784 | — | — | Plasmid |
| — | 788 | CaMV35S:ESPSss:aadA:35ST | Aminoglycoside | Selection |
| | | | adenylyltransfer | marker in |
| | | | ase | AR07-00 |
| — | 789 | GmU3:mCherry:RbcS-E9T | mCherry | Reporter gene |
| | | | | in AR07-00 |

### Plant Transformation: Bombardment

Pre-sterilized soybean seeds from soybean cultivar Jack were placed on an agar medium for germination. After an overnight incubation, embryonic axes (EAs) were aseptically isolated from seeds. The primary leaves of EAs were removed and the remaining EAs were treated with a solution containing BA (Benzyladenine) and GA (Gibberellic Acid) before they were wrapped in an aluminum foil and incubated in a growth chamber at 21° C. for up to 4 days.

On the day of transformation, gold particles coated with plasmid DNA that contains a mutated ALS (Acetolactate Synthase) gene and a gene coding for a recombinant protein were delivered into EAs using a Bio Rad biolistic apparatus. After the bombardment-mediated gene delivery, the targeted EAs were placed in a growth incubator for 1-3 days for recovery before they were transferred to a medium that contained 0.5 uM Imazapyr herbicide for shoot induction. Shoots which elongated from EAs were separated and were transferred to a rooting medium that contained 0.25 uM Imazapyr. Shoots that formed roots were transferred to Jiffy-7 peat pots for continuing development. Leaf tissues were collected and analyzed by ddPCR to evaluate the number of gene copies inserted.

### Plant Transformation: Agrobacterium

Pre-sterilized soybean seeds from cultivar Jack were placed on an agar medium for germination. After incubation in a growth chamber overnight, embryonic axes (EAs) were isolated aseptically from seeds. The primary leaves of EAs were removed and the remaining EAs were then stored in a refrigerator until use in transformation.

Two days before transformation, transferred glycerol stock of agrobacterium that contains a mutated ALS gene, a visible marker gene (mCherry), and a gene coding for a casein protein to a culture tube containing 3 ml LB medium and 100 μg/mg of kanamycin (LB Kan100), and placed the culture on a shaker at 250 rpm at 28° C. overnight. One day before transformation, 15 μl of overnight grown agrobacterium solution was inoculated into 30 ml of LB Kan100 medium and grown for 24 more hours. On the day of transformation, the O.D. (optical density) was adjusted to 0.5 in 50 ml infection medium supplemented with acetosyringone and dithiothreitol.

On the day of transformation, EAs separated in several sterile petri plates were co-cultivated in an incubator at 22° C. for 3 days with 15 ml of agrobacterium suspension. After co-cultivation, EAs were transferred to a shoot induction medium that contains 300 μg/ml cefotaxime and 0.5 μM Imazapyr. Elongated shoots from EAs were separated and transferred to a rooting medium that contained 0.2 μM Imazapyr. Shoots that formed roots and expressed mCherry gene were transferred to Jiffy-7 peat pots for continuing development. Leaf tissues were collected and analyzed by ddPCR to evaluate the number of gene copies inserted.

DNA Extraction and ddPCR Analysis

Total soybean genomic DNA was isolated from the first trifoliate leaves of transgenic events using the PureGene tissue DNA isolation kit (product #158667: QIAGEN, Valencia, CA, USA). Trifoliates were frozen in liquid nitrogen and pulverized. Cells were lysed using the PureGene Cell Lysis Buffer, proteins were precipitated using the PureGene Protein Precipitation Buffer, and DNA was precipitated from the resulting supernatant using ethanol. The DNA pellets were washed with 70% ethanol and resuspended in water.

Genomic DNA was quantified by the Quant-iT PicoGreen (product #P7589: ThermoFisher Scientific, Waltham, MA, USA) assay as described by manufacturer, and 150 ng of DNA was digested overnight with EcoRI, HindIII, NcoI, and/or KpnI, 30 ng of which was used for a BioRad ddPCR reaction, including labelled FAM or HEX probes for the transgene and Lectin1 endogenous gene respectively. Transgene copy number was calculated by comparing the measured transgene concentration to the reference gene concentration.

### RNA Isolation and Transcriptional Analysis

Transcript levels of transgenes in transgenic soybean seeds were determined by quantitative real-time PCR. Seeds were harvested at S1.09b stage (about 40 days after flowering), immediately frozen in liquid nitrogen and after grounding the RNA was isolated using the GeneJET Plant RNA purification kit (Catlog #K0802, Thermo Scientific). One microgram or less of RNA was treated with DNase I (Catlog #M0303S, NEB) prior to reverse transcription. Each RNA sample was diluted 25-fold and set up for SYBR green-based real-time quantitative PCR assays following the Luna® Universal One-Step RT-qPCR Kit (Catlog #E3005E, NEB). Real-time quantitative PCR assays were run in QuantStudio 6 Flex quantitative PCR system (Catlog #4485691, Applied Biosystems). For each transgenic event, four to six seeds were analyzed with 2 technical replicates.

The qPCR primer pairs were validated by building standard curves with four 10-fold serial dilutions. Only primer pairs showing over 90% amplification efficiency and generating a single peak in the dissociation curve were selected. The primers used are listed in Table 16. Gene expression levels were calculated using the Delta-Delta CT method (Vandesompele et al., 2002). Relative expressions of the seed storage gene GmGlyl to the constitutively expressed reference gene GmTUA5 were calculated as quality control. Samples showing less than one GmGly 1/GmTUA5 relative transcript expression indicated not mature seeds and those samples were excluded for further analysis. As our target gene expression cassettes are all driven by native seed storage gene promoters, we reported the transcript expressions of the target transgene in the format of “X native Glycinin1” by calculating the relative expression of transgene to the seed storage gene GmGlyl.

TABLE 16
Exemplary primer pairs utilized in qRT-PCR analysis

| Gene | Short | Forward Primer | Reverse Primer | Amplicon | Amplification |
|---|---|---|---|---|---|
| Name | Name | Sequence | Sequence | Size | Efficiency |
| Glycinin 1 | Seed2 | CTGAGTTTGGAT | ACTTGTATCAATG | 103 bp | 91.11% |
| | | CTCTCCGC | CCCGTCC | | |
| | | (SEQ ID NO: 732) | (SEQ ID NO: 733) | | |
| Alpha | TUA5 | GGATGTCAATGC | AACCTTAGCAAG | 134 bp | 90.42% |
| Tubulin 5 | | TGCTGTTG | GTCACCAC | | |
| | | (SEQ ID NO: 734) | (SEQ ID NO: 735) | | |
| Beta | OLG1 | ACGAACAAGGCA | AGGTGCTCGTACT | 101 bp | 91.99% |
| Lactoglobulin 1 | | TTGGCAGG | GGACACT | | |
| | | (SEQ ID NO: 736) | (SEQ ID NO: 737) | | |
| Beta | OLG2 | TCGATGCTCTCA | TCCTCACAAGGC | 123 bp | 103.47% |
| Lactoglobulin 2 | | ACGAGAACA | ATTGGCAAAC | | |
| | | (SEQ ID NO: 738) | (SEQ ID NO: 739) | | |
| Beta | OLG3 | AGACCAAAATTC | TGATTGTTCTGGC | 125 bp | 100.83% |
| Lactoglobulin 3 | | CTGCTGTGT | TCTGCGC | | |
| | | (SEQ ID NO: 740) | (SEQ ID NO: 741) | | |
| Beta | OLG4 | CTGGTCCTCGAC | AAGTGCCTCGTC | 123 bp | 107.94% |
| Lactoglobulin 4 | | ACTGATTATA | ATCAACCTC | | |
| | | (SEQ ID NO: 742) | (SEQ ID NO: 743) | | |
| Ovalbumin | OOVA | GCCGAGGAAAGA | TGTCTGGCTCTCT | 141 bp | 95.95% |
| 1 | L1 | TACCCCAT | ACCCAAGA | | |
| | | (SEQ ID NO: 744) | (SEQ ID NO: 745) | | |
| Ovalbumin | OOVA | AACGCTACCCTA | TGGCTCTCGACCC | 130 bp | 92.40% |
| 2 | L2 | TCCTGCCA | AAGAATTG | | |
| | | (SEQ ID NO: 746) | (SEQ ID NO: 747) | | |
| Ovalbumin | OOVA | CAGCAGACTGTA | TCAGCTCACGAG | 128 bp | 97.30% |
| 3 | L3 | CGCAGAAG | CCTGATCT | | |
| | | (SEQ ID NO: 748) | (SEQ ID NO: 749) | | |
| Ovalbumin | OOVA | AGGAGCTGCGTC | TGCACCCAAGTA | 127 bp | 101.35% |
| 4 | L4 | TATGGAGT | AACCATGGC | | |
| | | (SEQ ID NO: 750) | (SEQ ID NO: 751) | | |

### Protein Extraction and Detection: Preparation of Total Soluble Protein Samples

Total soluble soybean protein fractions were prepared from the seeds of transgenic events by bead beating seeds (seeds collected about 60-90 days after flowering) at 15000 rpm for 1 min. The resulting powder was resuspended in 50 mM Carbonate-Bicarbonate pH10.8, 1 mM DTT, 1×HALT Protease Inhibitor Cocktail (Product #78438 ThermoFisher Scientific). The resuspended powder was incubated at 4° C. for 15 minutes and then the supernatant collected after centrifuging twice at 4000 g, 20 min, 4° C. Protein concentration was measured using a modified Bradford assay (Thermo Scientific Pierce 660 nm assay; Product #22660 ThermoFisher Scientific) using a bovine serum albumin (BSA) standard curve.

### Recombinant Protein Quantification via ELISA

Wells of microtiter plates were coated directly with crude plant protein extract diluted in pH 9.5 Bicarbonate-Bicarbonate buffer and incubated overnight at 4° C. Microtiter plates were blocked with 3% BSA in phosphate buffered saline with 0.05% Tween-20, washed with phosphate buffered saline with 0.05% Tween-20, reacted with antigen specific antibody and subsequently reacted with HRP-conjugated sheep anti rabbit IgG (Product #AB6795 Abcam, Cambridge, UK). The reaction was visualized by the addition of chromogenic substrate (TMB) and reaction was stopped with 2M Sulfuric acid and absorbance read at 450 nm using BMG ClarioStar plate reader (Ortenberg, Germany). Recombinant protein from the seeds of transgenic events was quantified by a standard curve prepared from commercial reference protein spike-in standards.

### Example 7—Codon Variant Expression and RNA Secondary Structure

Results presented in earlier examples (including Example 3) revealed significant expression differences between codon-optimized variants of nucleic acids encoding for the same protein. For instance, Example 3 noted that β-lactoglobulin gene variant OLG 2 expressed at higher levels than other β-lactoglobulin codon variants.

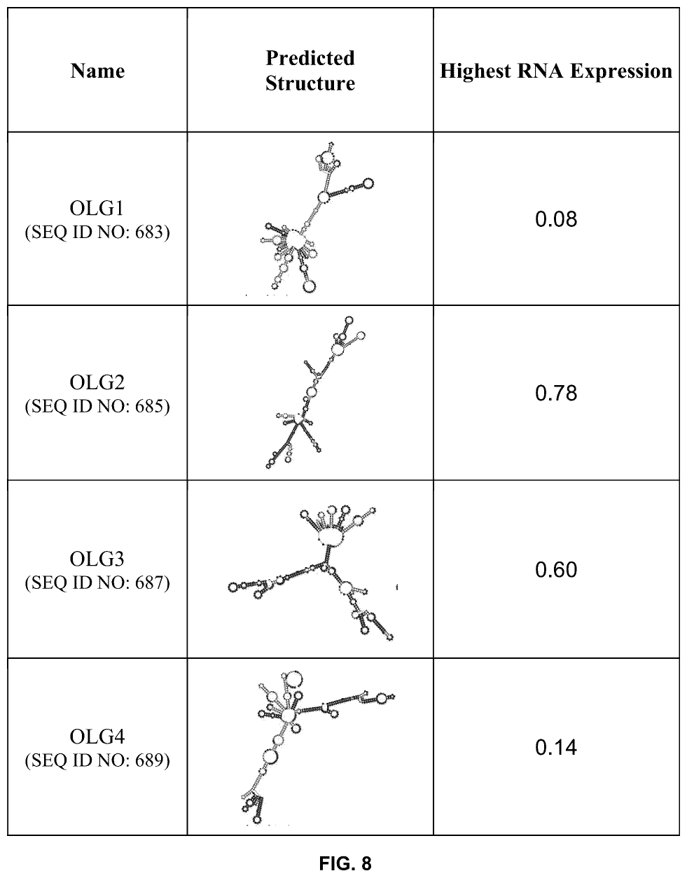

It was initially hypothesized that increased expression of some codon variants may correlate with the nucleic acid taking on a specific RNA secondary structure. However, a comparison of predicted structures suggested significant structural differences, even amongst higher expressing variants. See e.g., FIG. 8. Therefore no clear correlation was identified between predicted structure and expression.

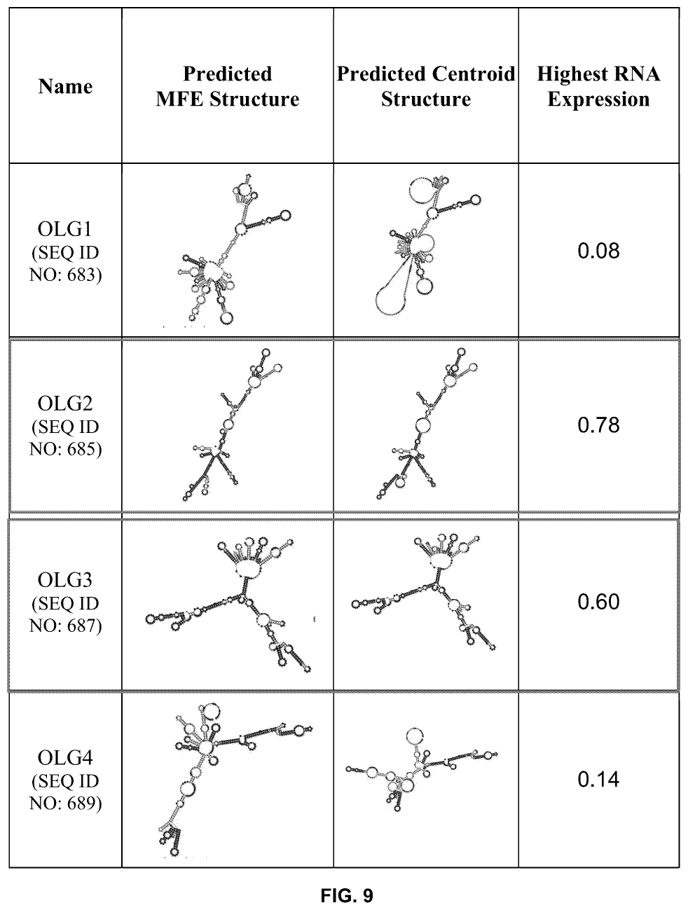

While conducting the structural analysis discuss above, the inventor(s) noticed that highly-expressing codon variants tended to have similar—if not identical—predicted structures across multiple RNA folding tools. For example, among the β-lactoglobulin codon variants discussed in this example, OLG2 and OLG3 exhibited the highest RNA expression, and also returned similar predicted secondary structures using the minimum free energy (MFE) and centroid models. See FIG. 9.

It was therefore hypothesized that RNA expression could be correlated to a codon variant exhibiting similar predicted secondary structures across multiple RNA folding models.

### Example 8—Nucleic Acid Optimization Through Secondary Structure Modeling

Analyses conducted in Example 8 suggested that RNA expression could be associated with predicted secondary structures across multiple RNA folding models. This Example further tests this hypothesis and evaluates whether structural similarities among predicted secondary structures could be predictive of RNA expression.

To test this hypothesis, 3-4 codon optimized variants were produced for β-lactoglobulin (OLG), Ovalbumin (OOVAL), and Green Fluorescent Protein (eGFP). Each of these optimized codon variant nucleic acid sequences were then analyzed in silico to generate predicted secondary structures. Two secondary structures based on two different RNA folding models were generated for each nucleic acid sequence using the RNAfold program. The MFE generated secondary structure represents the optimal secondary structure. The centroid generated secondary structure represents the minimum total base pair distance to all the structures in the thermodynamic ensemble.

### Structure Similarity by Visual Inspection

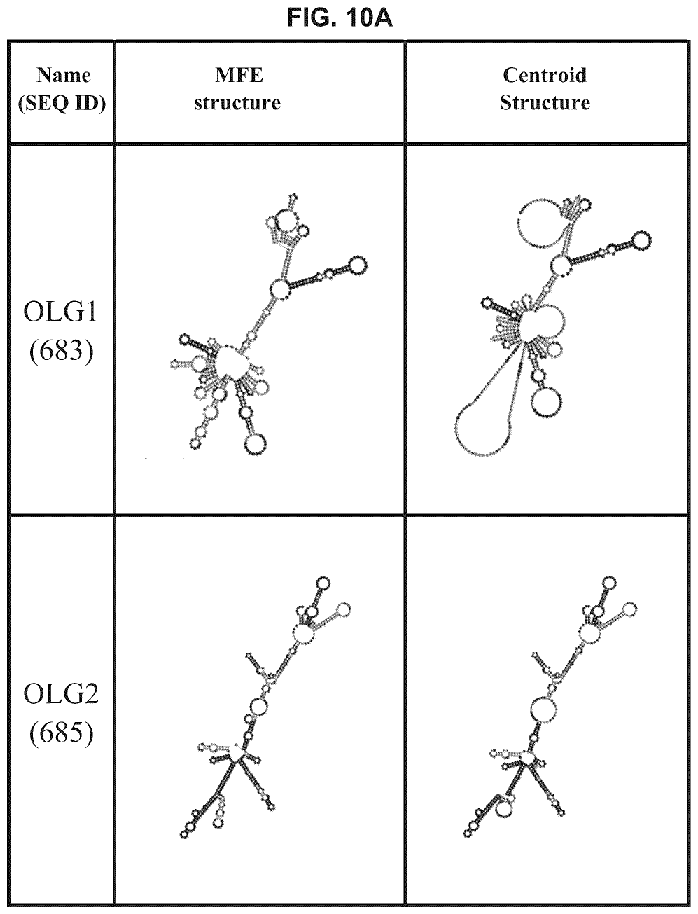

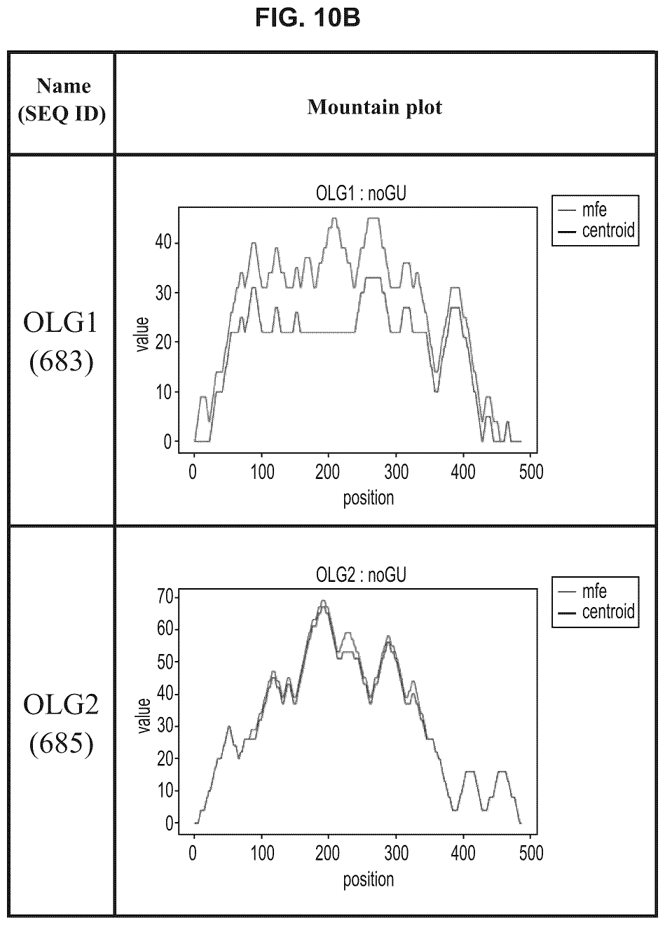

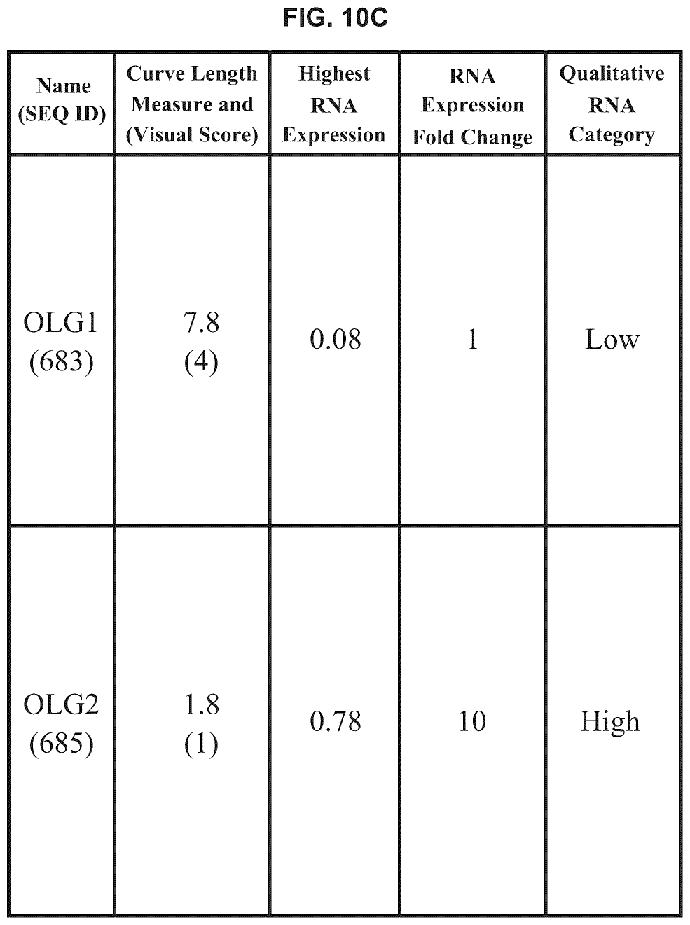

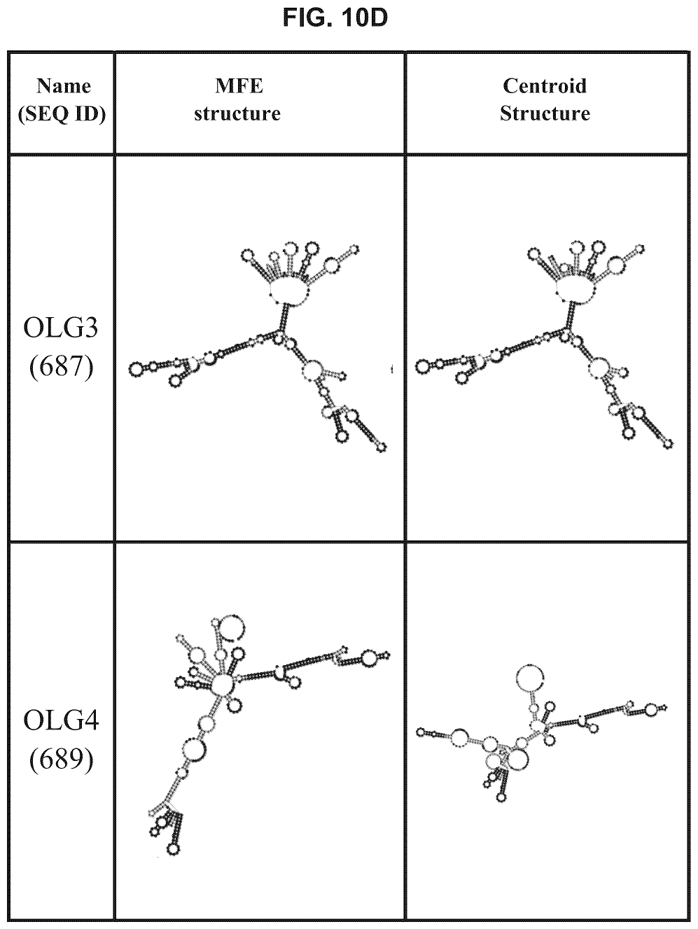

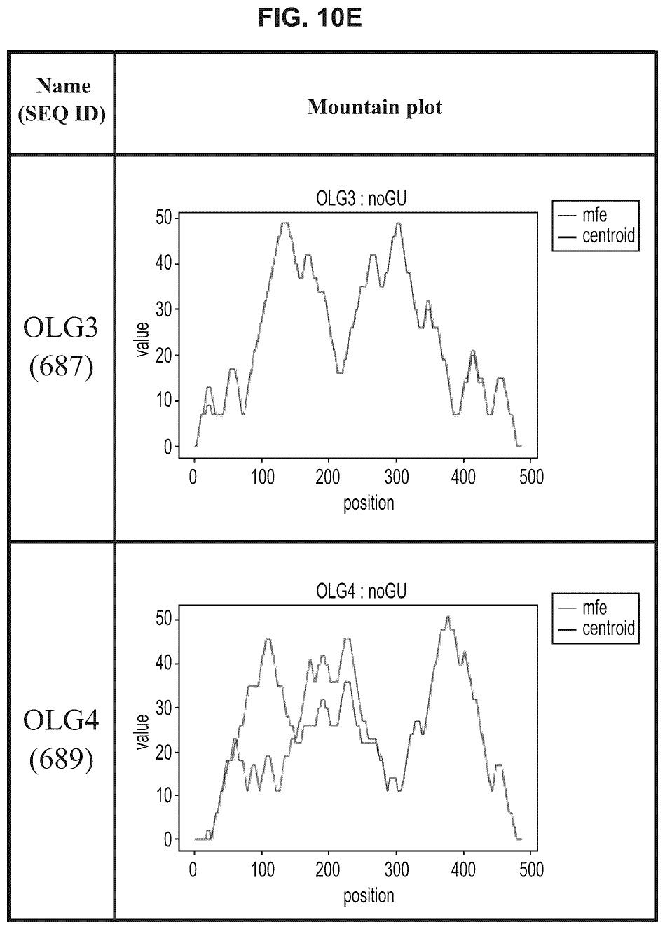

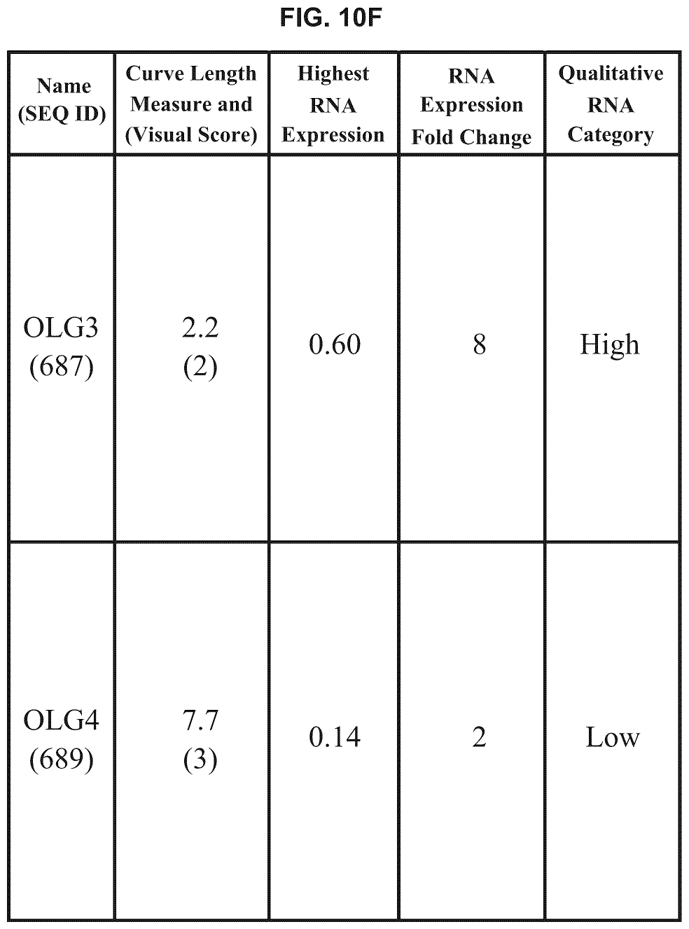

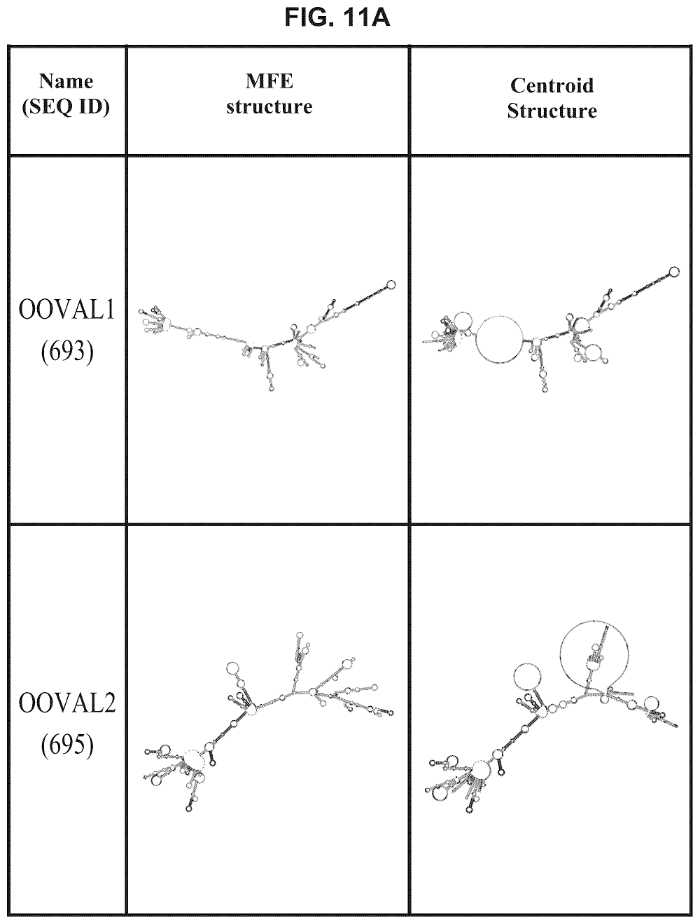

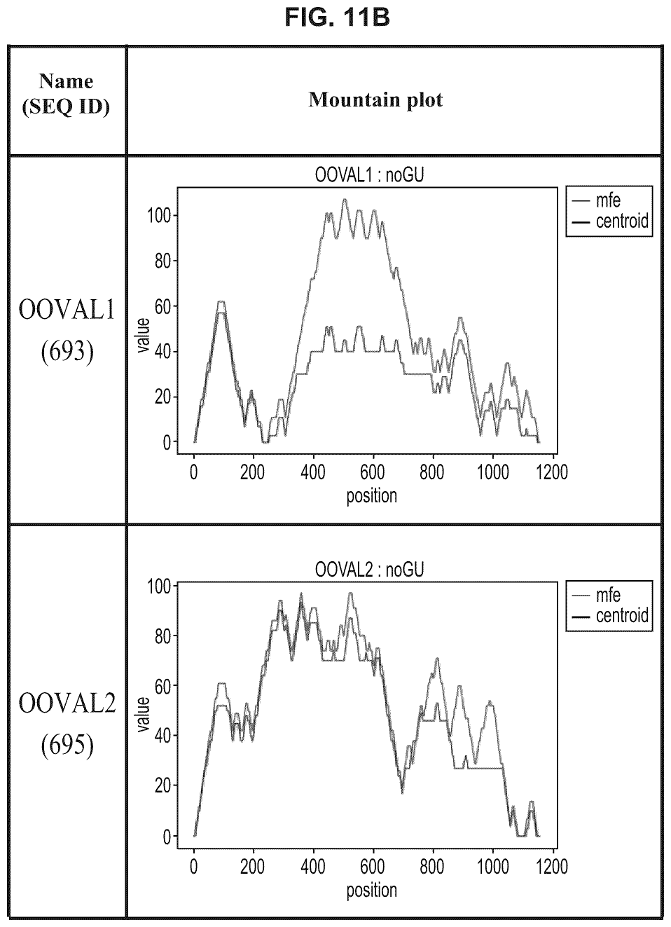

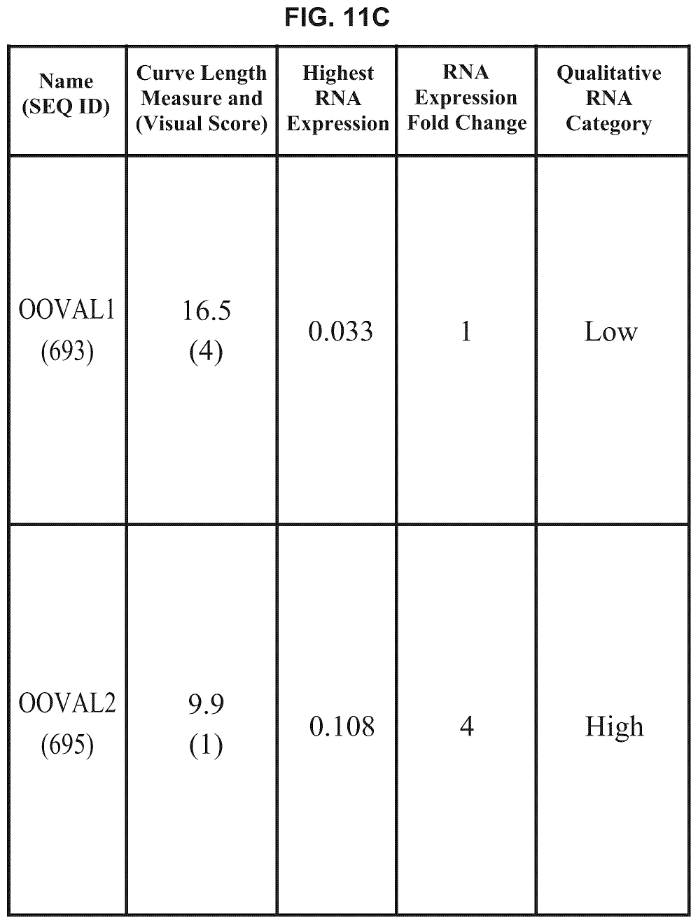

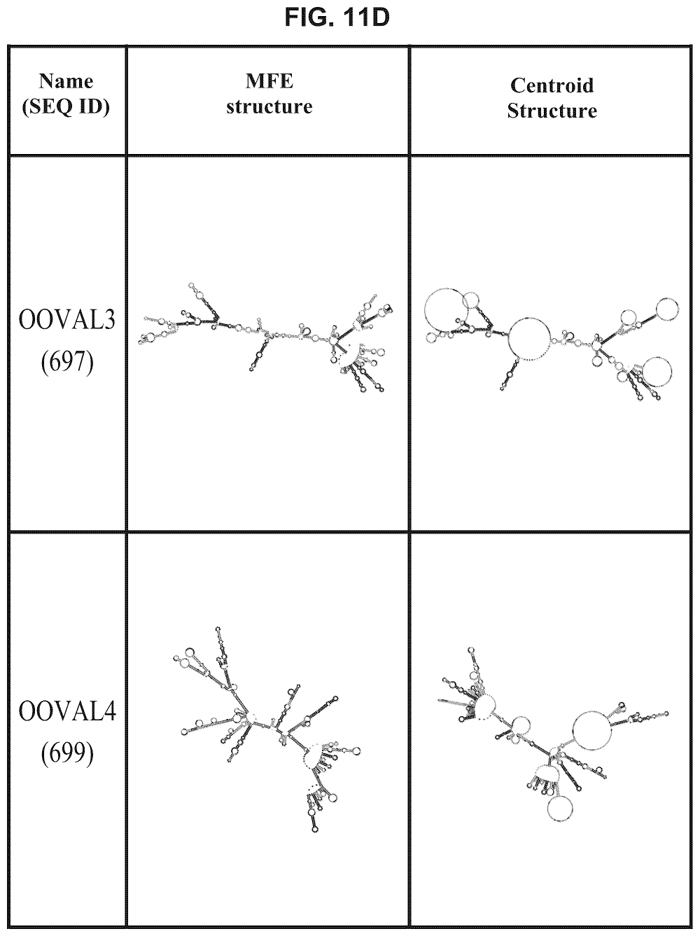

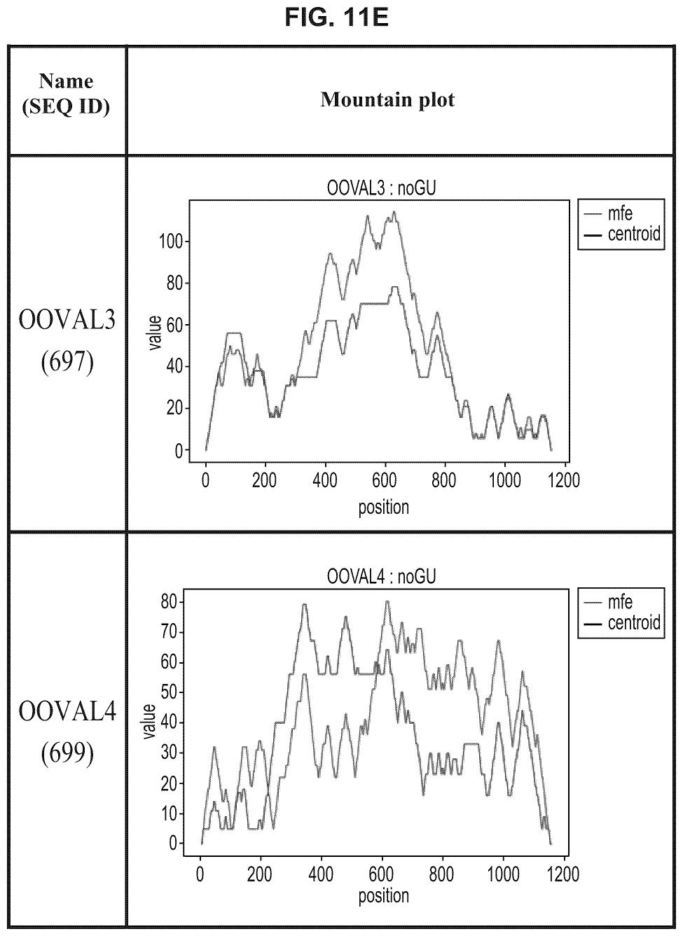

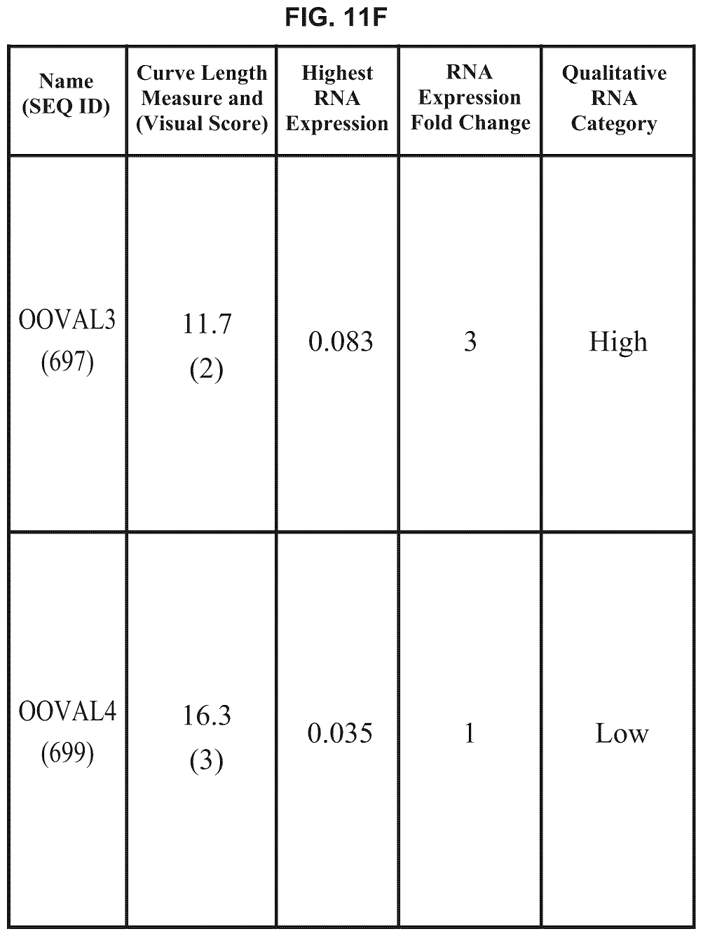

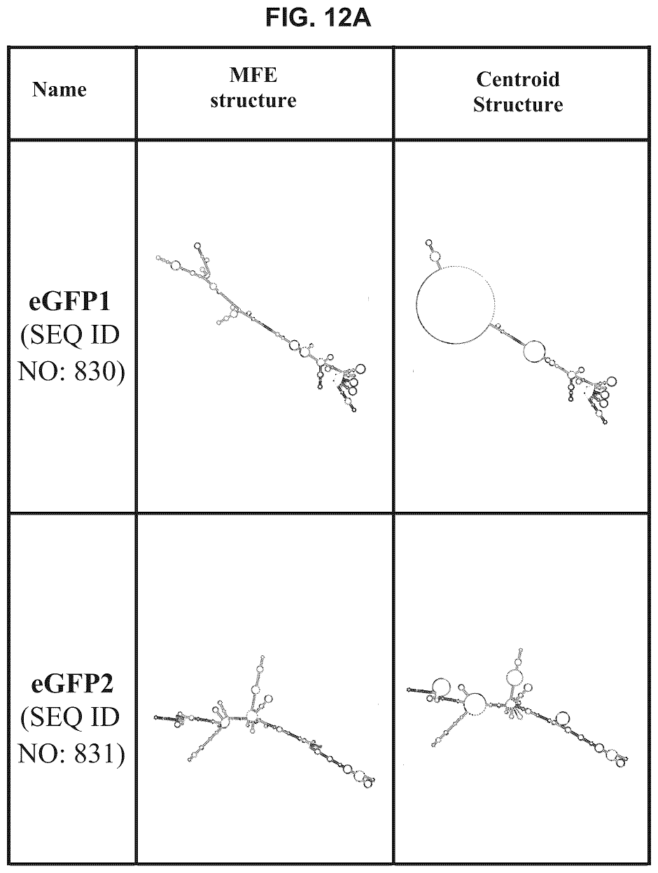

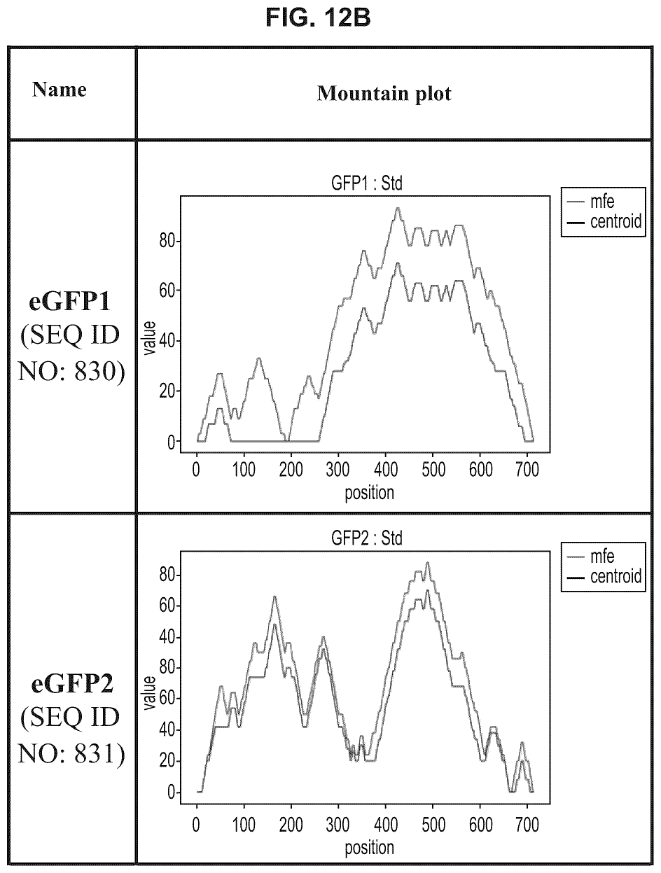

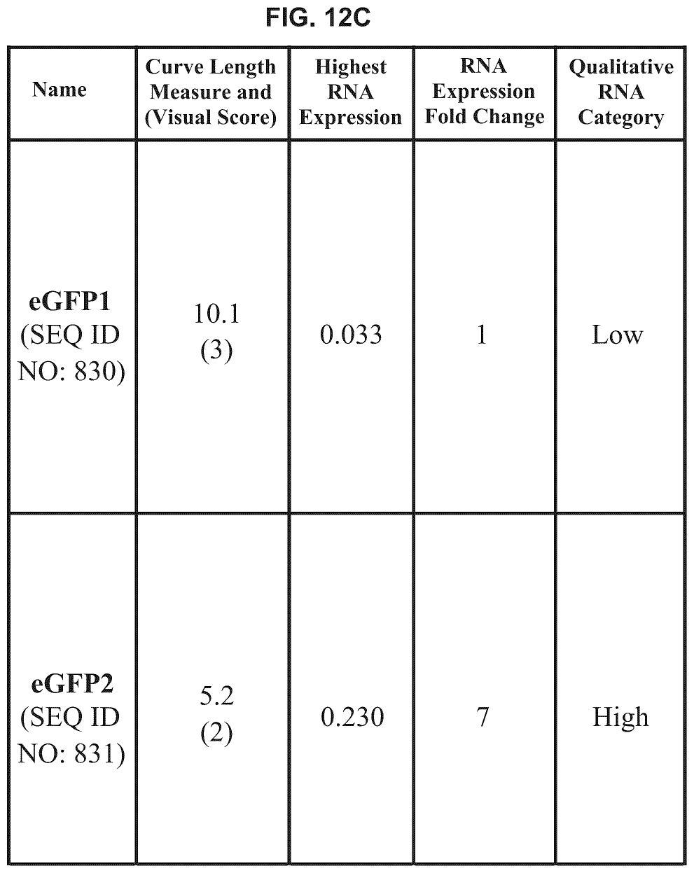

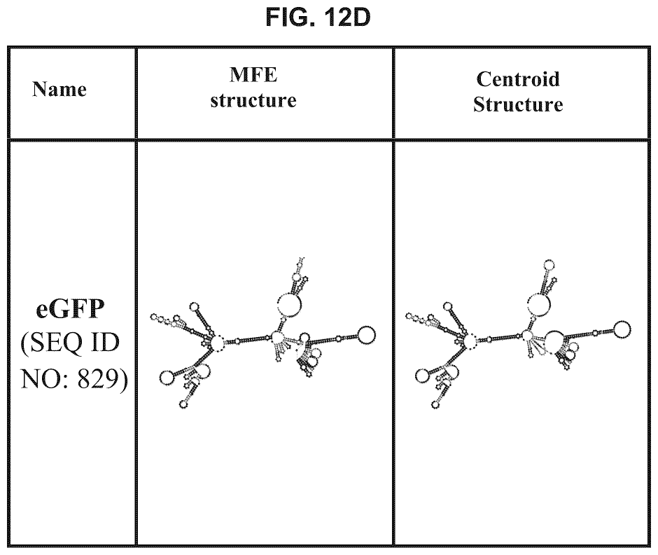

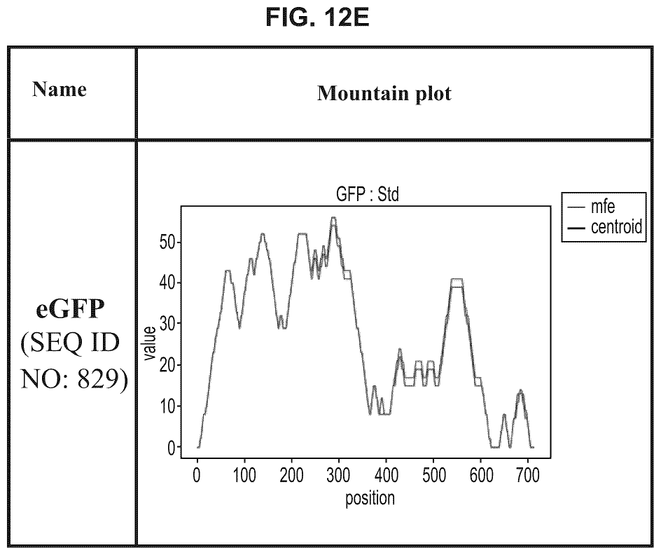

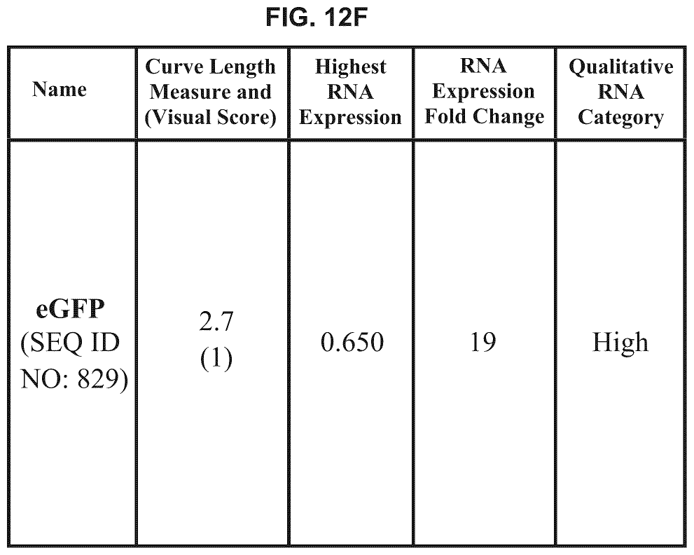

As an initial step, the similarity of the predicted MFE and centroid structures was visually compared and ranked according to perceived similarity. This similarity was assessed by stacking the secondary structure figures with 50% translucency in Microsoft Word and assessing the amount of overlap. Pictures of each of the predicted secondary structures, as well as the visual score for similarity of the two structures produced for each nucleic acid sequence is provided in FIGS. 10-12.

### Structure Similarity In Silico

As an alternative to visual inspection, an in silico structural similarity score was developed. Specifically, similarity between the two predicted secondary structures was calculated using the ViennaRNA Package (version 2.5.0) (world wide web-tbi.univie.ac.at/RNA/) (Gruber, 2008) and similarity measures package (pypi.org/project/similaritymeasures/) (Jekel, 2019).

The MFE structure was predicted by the minimum free energy algorithm of (Zuker & Stiegler 1981). The centroid structure was predicted by the suboptimal folding algorithm of (Wuchty et. al 1999).

The ViennaRNA package was first used to convert each of the predicted secondary structures to a height versus position plot (mountain plot), where the vertical y-axis height m(k) is given by the number of base pairs enclosing the base position in the horizontal x-axis (k). In general, this visualization of secondary structure depicts hairpin loops as plateaus and helices as slopes. The mountain plots further assisted in visualizing structural differences, where similar structures were visualized as overlapping mountain plot curves for the MFE and centroid structures, whereas different portions of the secondary structure were visualized as non-overlapping curves. See FIGS. 10-12. These mountain plots also permitted for visual assessment of structure similarity, which largely corresponded with the visual assessment conducted earlier in the Example.

In order to obtain a purely in silico similarity score, the mountain plot curves generated above were further analyzed by the python package similaritymeasures 0.4.4 (pypi.org/project/similaritymeasures/) (Jekel, 2019). This package was used to assess the curve length of each mountain plot, quantifying the deviation between the curves produced by the MFE and centroid secondary structures. Lower curve length measures indicated high overlap between the two curves, suggesting increased similarity between the two plotted secondary structures. Higher curve length measures indicated lower overlap between the curves, suggesting lower similarity between the two plotted secondary structures. These scores were then saved and are presented in FIGS. 10-12.

### Empirical Expression Measurement

The sequences in this example were then expressed in soybean using the methods described in Example 8. Briefly, each of the codon-optimized nucleic acid sequences were manufactured into nucleic acids and were cloned into expression vectors. The expression vectors were introduced into soybean, and RNA expression was measured via quantitative RT PCR.

### Nucleic Acid Predicted Structure Similarity as Predictor of Expression

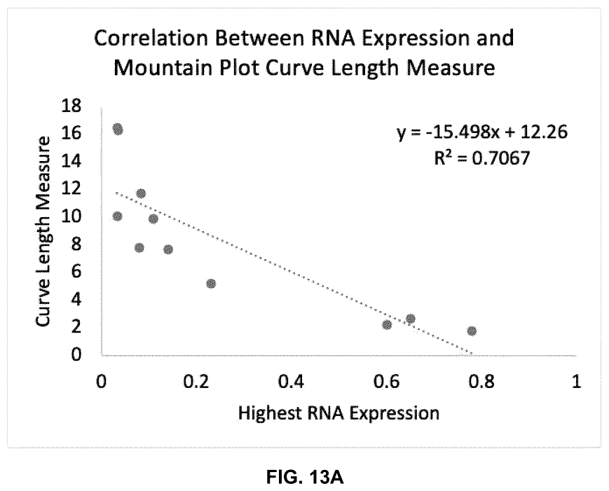

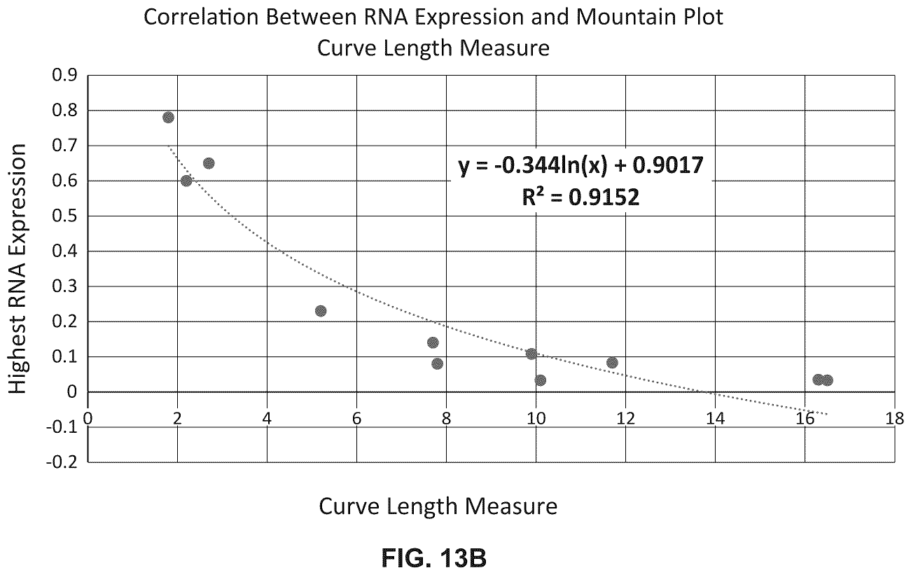

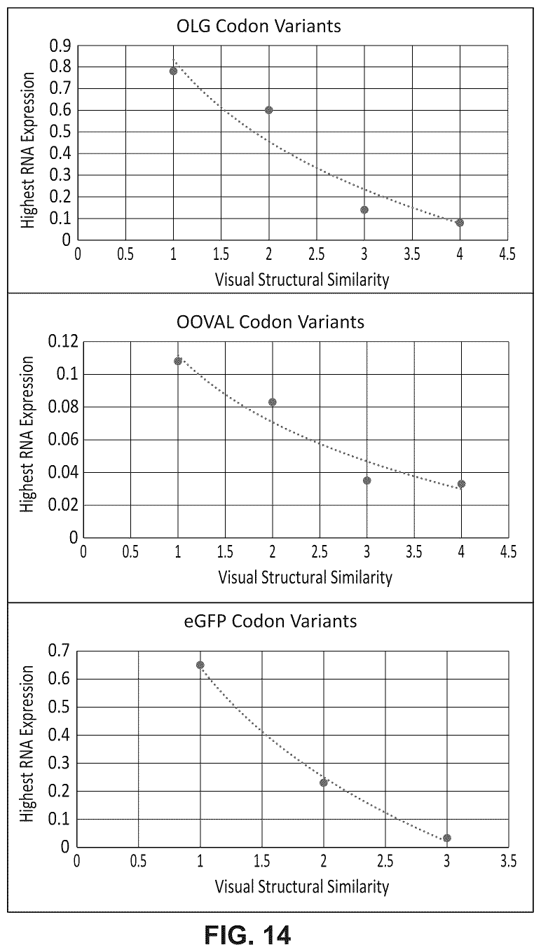

The in silico structural similarity scores measured above were plotted against the empirical expression data measured for each nucleic acid sequence (FIGS. 13A-B). The results demonstrated a strong correlation between structural similarity of predicted secondary structures and empirical expression. This correlation appeared to be logarithmic, with an R² correlation coefficient of 0.915. This correlation was measured across multiple codon variants and genes, demonstrating that it is not an artifact of any specific sequence. The general trend of correlation also held true for visual structural similarity scores, demonstrating that multiple structure similarity comparators can be used (FIG. 14).

### Milk Protein Sequences

The following Table 17 describes various representative species of milk proteins exemplified in the disclosure.

TABLE 17
Exemplary Milk Protein Sequences of the Disclosure

| | | | |
|---|---|---|---|
| SEQ | | | |
| ID NO | Description | Genus/species | Accession Number |

Kappa casein sequences

| | | | |
|---|---|---|---|
| 3 | Optimized kappa-casein | Artificial (codon optimized Bos | |
| | truncated version 1 | taurus) | |
| | (OKC1-T) | | |
| 4 | Optimized kappa-casein | Bos taurus | |
| | truncated version 1 | | |
| | (OKC1-T) | | |
| 85 | Kappa casein | Capra hircus | |
| 86 | Kappa casein | Ovis aries | |
| 87 | Kappa casein | Bubalus bubalis | |
| 88 | Kappa casein | Camelus dromedaries | |
| 89 | Kappa casein | Camelus bactrianus | |
| 90 | Kappa casein | Bos mutus | |
| 91 | Kappa casein | Equus caballus | |
| 92 | Kappa casein | Equus asinus | |
| 93 | Kappa casein | Rangifer tarandus | |
| 94 | Kappa casein | Alces alces | |
| 95 | Kappa casein | Vicugna pacos | |
| 96 | Kappa casein | Bos indicus | |
| 97 | Kappa casein | Lama glama | |
| 98 | Kappa casein | Homo sapiens | |
| 148 | Kappa casein | Bos taurus | NP_776719.1 |
| 149 | | | AAI02121.1 |
| 150 | | | AAA30433.1 |
| 151 | | | AAB26704.1 |
| 152 | | | 1406275A |
| 153 | | | AAF72097.1 |
| 154 | | | AAD32139.1 |
| 155 | | | XP_024848756.1 |
| 156 | | | CAF03625.1 |
| 157 | | | ABN42697.1 |
| 158 | | | AAD32140.1 |
| 159 | | | ALC76014.1 |
| 160 | | | DAA28589.1 |
| 161 | | | ADT82665.1 |
| 162 | | | ADT82666.1 |
| 163 | | | CAH56573.1 |
| 164 | | | ADT82669.1 |
| 165 | Kappa casein | Capra hircus | QIZ03342.1 |
| 166 | | | AYN74373.1 |
| 167 | | | AAM12026.1 |
| 168 | | | AFZ92921.1 |
| 169 | | | NP_001272516.1 |
| 170 | | | AAM12027.1 |
| 171 | | | AAR06605.1 |
| 172 | | | AAL90873.1 |
| 173 | | | AFZ92919.1 |
| 174 | | | QIZ03345.1 |
| 175 | | | AAR91623.1 |
| 176 | | | AAK17010.1 |
| 177 | | | AAL93193.1 |
| 178 | | | AFZ92918.1 |
| 179 | | | AAL90872.1 |
| 180 | | | AFZ92917.1 |
| 181 | | | AA039432.1 |
| 182 | | | AAL90871.1 |
| 183 | | | AA039431.1 |
| 184 | Kappa casein | Ovis aries | NP_001009378.1 |
| 185 | | | AAP69943.1 |
| 186 | Kappa casein | Bubalus bubalis | NP_001277901.1 |
| 187 | | | AXE74388.1 |
| 188 | | | APQ30586.1 |
| 189 | | | AXE74385.1 |
| 190 | | | XP_006071184.1 |
| 191 | | | AXE74386.1 |
| 192 | Kappa casein | Bos mutus | XP_005897104.1 |
| 193 | | | XP_014334109.1 |
| 194 | | | MXQ92034.1 |
| 195 | Kappa casein | Bos indicus | XP_019818432.1 |
| 196 | | | ACF15188.1 |
| 197 | | | ACF15186.1 |
| 198 | | | ACF15190.1 |
| 199 | | | ABY81250.1 |
| 200 | | | ABY81251.1 |
| 201 | | | ADT82668.1 |
| 202 | | | ADT82663.1 |
| 203 | | | ADT82671.1 |
| 204 | | | ADT82670.1 |
| 205 | | | AAQ73171.1 |
| 206 | Kappa casein | Jeotgalicoccus coquinae | WP_188357548.1 |
| 207 | (Hypothetical Protein) | | WP_188357549.1 |
| 208 | Kappa casein isoform X1 | Bison bison bison | XP_010837415.1 |
| 209 | | | XP_010837416.1 |
| 210 | Kappa casein | Bos grunniens | AFM93768.1 |
| 211 | | | AXE74296.1 |
| 212 | | | AAM25910.1 |
| 213 | | | ABU53615.1 |
| 214 | | | AAM25909.1 |
| 215 | | | AAF63191.1 |
| 216 | Kappa casein | Bos indicus × Bos taurus | AAF72096.1 |
| 217 | | | AAF72098.1 |
| 218 | Kappa casein (precursor) | Oreamnos americanus | P50423.1 |
| 219 | Kappa casein (precursor) | Naemorhedus goral | P50422.1 |
| 220 | Kappa casein | Odocoileus virginianus texanus | XP_020729185.1 |
| 221 | Kappa casein (precursor) | Capricornis sumatraensis | P50420.1 |
| 222 | Kappa casein (precursor) | Capricornis crispus | BAA03287.1 |
| 223 | | | P42156.1 |
| 224 | Kappa casein (precursor) | Capricornis swinhoei | P50421.1 |
| 225 | Kappa casein (precursor) | Saiga tatarica | P50425.1 |
| 226 | Kappa casein (precursor) | Rupicapra rupicapra | P50424.1 |
| 227 | Kappa casein (precursor) | Cervus nippon | P42157.1 |
| 228 | Kappa casein | Bos frontalis | ADF58295.1 |
| 229 | Kappa casein | Muntiacus reevesi | KAB0354473.1 |
| | (hypothetical protein | | |
| | FD755 023011) | | |
| 230 | Kappa casein | Muntiacus muntjak | KAB0341224.1 |
| | (hypothetical protein | | |
| | FD754 018150) | | |
| 231 | Kappa casein | Madoqua saltiana | AFY03578.1 |
| 232 | Kappa casein | Gazella dorcas | AFY03574.1 |
| 233 | Kappa casein | Gazella arabica | AFY03576.1 |
| 234 | Kappa casein | Capra ibex ibex | AAP80529.1 |
| 235 | Kappa casein | Ovis ammon severtzovi | ADB66396.1 |
| 236 | Kappa casein | Ovis orientalis gmelini | ADB66423.1 |
| 237 | | | ADB66420.1 |
| 238 | Kappa casein | Cervus hanglu yarkandensis | KAF4013038.1 |
| | (hypothetical protein | | |
| | G4228 004474) | | |
| 239 | Kappa casein | Procapra gutturosa | AFY03581.1 |
| 240 | | | AFY03580.1 |
| 1 | Optimized para-kappa- | Artificial (codon optimized Bos | |
| | casein truncated version | taurus) | |
| | 1 (paraOKC1-T) | | |
| 2 | Optimized para-kappa- | Bos taurus | |
| | casein truncated version | | |
| | 1 (paraOKC1-T) | | |
| 241 | Kappa casein isoform X1 | Bos taurus | AAA30433.1 |
| 242 | | | 1406275A |
| 243 | | | AAI02121.1 |
| 244 | | | NP_776719.1 |
| 245 | | | DAA28589.1 |
| 246 | | | AAB26704.1 |
| 247 | | | XP_024848756.1 |
| 248 | | | ABN42697.1 |
| 249 | | | AAF72097.1 |
| 250 | | | 721588A |
| 251 | | | AAD32139.1 |
| 252 | | | AAD32140.1 |
| 253 | | | CAF03625.1 |
| 254 | Kappa casein | Jeotgalicoccus coquinae | WP_188357548.1 |
| 255 | (hypothetical protein) | | WP_188357549.1 |
| 256 | Kappa casein isoform X1 | Bos mutus | XP_005897104.1 |
| 257 | | | XP_014334109.1 |
| 258 | | | MXQ92034.1 |
| 259 | Kappa casein | Bos indicus | XP_019818432.1 |
| 260 | | | ACF15188.1 |
| 261 | | | ABY81250.1 |
| 262 | | | ABY81251.1 |
| 263 | | | ACF15186.1 |
| 264 | | | ACF15190.1 |
| 265 | | | ADT82668.1 |
| 266 | Kappa casein | Bos grunniens | AXE74296.1 |
| 267 | | | AFM93768.1 |
| 268 | | | AAM25910.1 |
| 269 | | | AAM25909.1 |
| 270 | | | ABU53615.1 |
| 271 | Kappa casein isoform X1 | Bison bison bison | XP_010837415.1 |
| 272 | | | XP_010837416.1 |
| 273 | Kappa casein (precursor) | Bubalus bubalis | NP_001277901.1 |
| 274 | | | XP_006071184.1 |
| 275 | | | AXE74388.1 |
| 276 | | | AXE74385.1 |
| 277 | | | APQ30586.1 |
| 278 | | | AXE74386.1 |
| 279 | Kappa casein (precursor) | Oreamnos americanus | P50423.1 |
| 280 | Kappa casein (precursor) | Capricornis swinhoei | P50421.1 |
| 281 | Kappa casein (precursor) | Naemorhedus goral | P50422.1 |
| 282 | Kappa casein (precursor) | Capricornis sumatraensis | P50420.1 |
| 283 | Kappa casein (precursor) | Capricornis crispus | BAA03287.1 |
| 284 | | | P42156.1 |
| 285 | Kappa casein (precursor) | Saiga tatarica | P50425.1 |
| 286 | Kappa casein | Bos indicus × Bos taurus | AAF72096.1 |
| 287 | | | AAF72098.1 |
| 288 | Kappa casein (precursor) | Capra hircus | NP_001272516.1 |
| 289 | | | AYN74373.1 |
| 290 | | | QIZ03345.1 |
| 291 | | | QIZ03342.1 |
| 292 | | | AFZ92921.1 |
| 293 | | | AAR06605.1 |
| 294 | | | AAM12026.1 |
| 295 | | | AAL93193.1 |
| 296 | | | AAR91623.1 |
| 297 | | | AFZ92917.1 |
| 298 | | | AAM12027.1 |
| 299 | | | AAL90873.1 |
| 300 | | | AFZ92918.1 |
| 301 | | | AAL90871.1 |
| 302 | | | AAL90872.1 |
| 303 | | | AAL31535.1 |
| 304 | | | AAL31534.1 |
| 305 | | | ABK59545.1 |
| 306 | | | AAO39432.1 |
| 307 | | | AFZ92919.1 |
| 308 | | | AAK17010.1 |
| 309 | | | AA039431.1 |
| 310 | | | AAP80475.1 |
| 311 | Kappa casein | Odocoileus virginianus texanus | XP 020729185.1 |
| 312 | Kappa casein (precursor) | Rupicapra rupicapra | P50424.1 |
| 313 | Kappa casein (precursor) | Ovis aries | NP 001009378.1 |
| 314 | | | AAP69943.1 |
| 315 | Kappa casein (precursor) | Cervus nippon | P42157.1 |
| 316 | Kappa casein | Gazella arabica | AFY03576.1 |
| 317 | Kappa casein | Muntiacus muntjak | KAB0341224.1 |
| | (hypothetical protein | | |
| | FD754 018150) | | |
| 318 | Kappa casein | Muntiacus reevesi | KAB0354473.1 |
| | (hypothetical protein | | |
| | FD755 023011) | | |
| 319 | Kappa casein | Gazella dorcas | AFY03575.1 |
| 320 | Kappa casein | Procapra gutturosa | AFY03581.1 |
| 321 | | | AFY03580.1 |
| 322 | Kappa casein | Madoqua saltiana | AFY03578.1 |
| 323 | Kappa casein | Ammotragus lervia | QIN85723.1 |
| 324 | | | QIN85720.1 |
| 325 | | | QIN85721.1 |
| 326 | Kappa casein | Capra sibirica | AAP80568.1 |
| 327 | Kappa casein | Ovis canadensis canadensis | ADB66397.1 |
| 328 | | | ADB66402.1 |
| 329 | Kappa casein | Gazella subgutturosa marica | AFY03577.1 |
| 330 | Kappa casein | Antilope cervicapra | AFY03573.1 |
| 331 | Kappa casein | Capra ibex ibex | AAP80529.1 |
| 332 | Kappa casein | Ovis vignei arkal | ADB66436.1 |
| 333 | | | ADB66442.1 |
| 334 | Kappa casein | Ovis ammon collium | ADB66395.1 |
| 335 | Kappa casein | Ovis vignei blanfordi | ADB66445.1 |
| 336 | Kappa casein | Ovis orientalis gmelini | ADB66423.1 |
| 337 | | | ADB66420.1 |
| 338 | Kappa casein | Ovis orientalis × vignei | ADB66465.1 |
| 339 | Kappa casein | Ovis vignei vignei | ADB66456.1 |
| 340 | Kappa casein | Ovis ammon severtzovi | ADB66396.1 |

Alpha S1 casein sequences

| | | | |
|---|---|---|---|
| 7 | Optimized alpha S1- | Artificial (codon optimized Bos | |
| | casein truncated version | taurus) | |
| | 1(OaS1-T) | | |
| 8 | Optimized alpha S1- | Bos taurus | |
| | casein truncated version | | |
| | 1(OaS1-T) | | |
| 99 | Alpha S1 casein | Capra hircus | |
| 100 | Alpha S1 casein | Ovis aries | |
| 101 | Alpha S1 casein | Bubalus bubalis | |
| 102 | Alpha S1 casein | Camelus dromedaries | |
| 103 | Alpha S1 casein | Camelus bactrianus | |
| 104 | Alpha S1 casein | Bos mutus | |
| 105 | Alpha S1 casein | Equus caballus | |
| 106 | Alpha S1 casein | Equus asinus | |
| 107 | Alpha S1 casein | Bos indicus | |
| 108 | Alpha S1 casein | Lama glama | |
| 109 | Alpha S1 casein | Homo sapiens | |
| 341 | Alpha S1 casein | Bos taurus | ABW98943.1 |
| 342 | | | XP_024848771.1 |
| 343 | | | ABW98940.1 |
| 344 | | | ACG63494.1 |
| 345 | | | XP_015327132.1 |
| 346 | | | XP_024848772.1 |
| 347 | | | 1308122A |
| 348 | | | ABW98949.1 |
| 349 | | | AAA30429.1 |
| 350 | | | XP_015327135.1 |
| 351 | | | XP_015327134.1 |
| 352 | | | XP_024848773.1 |
| 353 | | | XP_015327133.1 |
| 354 | | | XP_024848774.1 |
| 355 | | | XP_015327136.1 |
| 356 | | | XP_024848775.1 |
| 357 | | | XP_005208084.1 |
| 358 | | | XP_024848776.1 |
| 359 | | | XP_015327137.1 |
| 360 | | | XP_015327138.1 |
| 361 | | | XP_024848777.1 |
| 362 | | | XP_024848778.1 |
| 363 | | | XP_015327139.1 |
| 364 | | | ABW98944.1 |
| 365 | | | XP_015327140.1 |
| 366 | | | XP_024848779.1 |
| 367 | | | XP_015327141.1 |
| 368 | | | XP_024848780.1 |
| 369 | | | XP_015327142.1 |
| 370 | | | ABW98945.1 |
| 371 | | | XP_024848782.1 |
| 372 | | | ABW98951.1 |
| 373 | | | XP_024848784.1 |
| 374 | | | XP_024848783.1 |
| 375 | | | ABW98950.1 |
| 376 | | | ABW98941.1 |
| 377 | | | XP_005208086.1 |
| 378 | | | ABW98942.1 |
| 379 | | | ABW98937.1 |
| 380 | | | ABW98952.1 |
| 381 | | | ABW98954.1 |
| 382 | | | ABW98953.1 |
| 383 | | | ABW98955.1 |
| 384 | | | ABW98957.1 |
| 385 | Alpha S1 casein | Capra hircus | XP_017904616.1 |
| 386 | | | QIZ03312.1 |
| 387 | | | ALJ30147.1 |
| 388 | | | P18626.2 |
| 389 | | | XP_017904617.1 |
| 390 | | | AFN44013.1 |
| 391 | | | QIZ03319.1 |
| 392 | | | CAA51022.1 |
| 393 | | | NP_001272624.1 |
| 394 | | | ALJ30148.1 |
| 395 | | | QIZ03317.1 |
| 396 | | | QIZ03310.1 |
| 397 | | | QIZ03318.1 |
| 398 | | | XP_017904618.1 |
| 399 | | | XP_017904620.1 |
| 400 | | | XP_017904619.1 |
| 401 | | | XP_017904621.1 |
| 402 | | | XP_017904622.1 |
| 403 | Alpha S1 casein | Ovis aries | XP_012034747.1 |
| 404 | | | P04653.3 |
| 405 | | | AAB34797.1 |
| 406 | | | ACJ46472.1 |
| 407 | | | XP 027826521.1 |
| 408 | | | XP 027826520.1 |
| 409 | | | ACR58469.1 |
| 410 | | | ACJ46473.1 |
| 411 | | | AAB34798.1 |
| 412 | | | NP_001009795.1 |
| 413 | Alpha S1 casein | Bubalus bubalis | AAZ14098.1 |
| 414 | | | APQ30583.1 |
| 415 | | | 062823.2 |
| 416 | | | XP_006071187.1 |
| 417 | | | QCP57314.1 |
| 418 | | | XP_025145744.1 |
| 419 | | | QPO15022.1 |
| 420 | | | XP_025145745.1 |
| 421 | | | ACJ14317.1 |
| 422 | | | XP_006071188.1 |
| 423 | | | XP_025145747.1 |
| 424 | | | XP_025145746.1 |
| 425 | | | XP_025145748.1 |
| 426 | | | XP_025145749.1 |
| 427 | | | XP_025145750.1 |
| 428 | | | XP_025145751.1 |
| 429 | | | XP_025145752.1 |
| 430 | | | XP_025145753.1 |
| 431 | Alpha S1 casein | Bos mutus | XP_005902100.1 |
| 432 | Alpha S1 casein | Bos indicus | XP_019818428.1 |
| 433 | Alpha S1 casein | Jeotgalicoccus coquinae | WP_188357546.1 |
| 434 | (hypothetical protein) | | GGE26809.1 |
| 435 | Alpha S1 casein | Bison bison bison | XP_010850445.1 |
| 436 | Alpha S1 casein | Bos grunniens | AXE74293.1 |
| 437 | Alpha S1 casein | Jeotgalicoccus aerolatus | WP_188349304.1 |
| 438 | (hypothetical protein) | | WP_188352531.1 |
| 439 | Alpha S1 casein | Muntiacus muntjak | KAB0341228.1 |
| | (hypothetical protein | | |
| | FD754 018154) | | |
| 440 | Alpha S1 casein | Muntiacus reevesi | KAB0354470.1 |
| | (hypothetical protein | | |
| | FD755 023008) | | |

Alpha S2 casein sequences

| | | | |
|---|---|---|---|
| 83 | Optimized alpha S2- | Artificial (codon optimized Bos | |
| | casein truncated version | taurus) | |
| | 1(OaS2-T) | | |
| 84 | Optimized alpha S2- | Bos taurus | |
| | casein truncated version | | |
| | 1(OaS2-T) | | |
| 110 | Alpha S2 casein | Capra hircus | |
| 111 | Alpha S2 casein | Ovis aries | |
| 112 | Alpha S2 casein | Bubalus bubalis | |
| 113 | Alpha S2 casein | Camelus dromedaries | |
| 114 | Alpha S2 casein | Camelus bactrianus | |
| 115 | Alpha S2 casein | Bos mutus | |
| 116 | Alpha S2 casein | Equus caballus | |
| 117 | Alpha S2 casein | Equus asinus | |
| 118 | Alpha S2 casein | Vicugna pacos | |
| 119 | Alpha S2 casein | Bos indicus | |
| 120 | Alpha S2 casein | Lama glama | |
| 441 | Alpha S2 casein | Bos taurus | AAI14774.1 |
| 442 | | | XP_024848786.1 |
| 443 | | | XP_015327143.1 |
| 444 | Alpha S2 casein | Capra hircus | QIS93310.1 |
| 445 | | | NP_001272514.1 |
| 446 | | | CAB94236.1 |
| 447 | | | QIS93322.1 |
| 448 | | | AAB32166.1 |
| 449 | | | QIS93306.1 |
| 450 | | | XP_013820127.2 |
| 451 | | | QIS93323.1 |
| 452 | | | QIZ03322.1 |
| 453 | | | QIS93316.1 |
| 454 | | | CAB59920.1 |
| 455 | | | CAC21704.2 |
| 456 | | | QIS93307.1 |
| 457 | | | XP_013820130.2 |
| 458 | | | QIS93319.1 |
| 459 | | | QIS93321.1 |
| 460 | | | XP_013820128.2 |
| 46 | | | QIS93304.1 |
| 462 | | | XP_013820129.2 |
| 463 | | | QIS93305.1 |
| 464 | | | QIS93314.1 |
| 465 | | | QIS93317.1 |
| 466 | | | XP_013820132.2 |
| 467 | | | XP_013820131.2 |
| 468 | Alpha S2 casein | Ovis aries | ADB65931.1 |
| 469 | | | NP_001009363.1 |
| 470 | | | ADB65933.1 |
| 471 | | | ADB65935.1 |
| 472 | | | ADB65934.1 |
| 473 | | | ADB65932.1 |
| 474 | Alpha S2 casein | Bubalus bubalis | NP_001277794.1 |
| 475 | | | AAZ80050.1 |
| 476 | | | CAA06534.2 |
| 477 | | | AFB69498.1 |
| 478 | | | XP_006071185.2 |
| 479 | | | AAZ57423.1 |
| 480 | | | APQ30584.1 |
| 481 | | | XP_025145302.1 |
| 482 | | | XP_025145301.1 |
| 483 | Alpha S2 casein | Bos mutus | XP_014335716.1 |
| 484 | | | ELR51813.1 |
| 485 | Alpha S2 casein | Jeotgalicoccus aerolatus | WP_188352530.1 |
| 486 | (hypothetical protein) | | GGE08804.1 |
| 487 | Alpha S2 casein | Jeotgalicoccus coquinae | WP_188357545.1 |
| | (hypothetical protein) | | |
| 488 | Alpha S2 casein | Bos grunniens | AXE74294.1 |
| 489 | Alpha S2 casein | Bison bison bison | XP_010850447.1 |
| 490 | Alpha S2 casein | Bos indicus × Bos taurus | XP_027401112.1 |
| 491 | Alpha S2 casein | Odocoileus virginianus texanus | XP_020729187.1 |
| 492 | Alpha S2 casein | Muntiacus muntjak | KAB0341229.1 |
| | (hypothetical protein | | |
| | FD754 018155) | | |
| 493 | Alpha S2 casein | Muntiacus reevesi | KAB0354254.1 |
| | (hypothetical protein | | |
| | FD755 022792) | | |
| 494 | Alpha S2 casein | Cervus elaphus | OWK13818.1 |
| | (CSN1S2) | hippelaphus | |

Beta-casein sequences

| | | | |
|---|---|---|---|
| 5 | Optimized beta-casein | Artificial (codon optimized Bos | |
| | truncated version 2 | taurus) | |
| | (OBC-T2) | | |
| 6 | Optimized beta-casein | Bos taurus | |
| | truncated version 2 | | |
| | (OBC-T2) | | |
| 121 | Beta casein | Capra hircus | |
| 122 | Beta casein | Ovis aries | |
| 123 | Beta casein | Bubalus bubalis | |
| 124 | Beta casein | Camelus dromedaries | |
| 125 | Beta casein | Camelus bactrianus | |
| 126 | Beta casein | Bos mutus | |
| 127 | Beta casein | Equus caballus | |
| 128 | Beta casein | Equus asinus | |
| 129 | Beta casein | Alces alces | |
| 130 | Beta casein | Vicugna pacos | |
| 131 | Beta casein | Bos indicus | |
| 132 | Beta casein | Lama glama | |
| 133 | Beta casein | Homo sapiens | |
| 495 | Beta casein | Bos taurus | AAB29137.1 |
| 496 | | | AAA30431.1 |
| 497 | | | 1314242A |
| 498 | | | AGT56763.1 |
| 499 | | | AAI11173.1 |
| 500 | | | XP_010804480.2 |
| 501 | | | AAA30430.1 |
| 502 | | | XP_015327157.2 |
| 503 | | | ABR10906.1 |
| 504 | | | ABL74247.1 |
| 505 | | | QCI03091.1 |
| 50€ | | | QCI03090.1 |
| 507 | | | CAC37028.1 |
| 508 | Beta casein | Capra hircus | P33048.1 |
| 509 | | | QIZ03333.1 |
| 510 | | | CAB39200.1 |
| 511 | | | AAK97639.1 |
| 512 | | | XP_005681778.2 |
| 513 | | | QLI42602.1 |
| 514 | | | XP_013820153.1 |
| 515 | | | QLI42606.1 |
| 516 | | | QHN12643.1 |
| 517 | | | ABQ52487.1 |
| 518 | | | QHN12642.1 |
| 519 | | | CAB39313.1 |
| 520 | | | QHN12644.1 |
| 521 | | | AWN06750.1 |
| 522 | Beta casein | Ovis aries | P11839.3 |
| 523 | | | NP_001009373.1 |
| 524 | Beta casein | Bubalus bubalis | QHB80269.1 |
| 525 | | | APQ30585.1 |
| 526 | | | QHB80272.1 |
| 527 | | | QHB80273.1 |
| 528 | | | NP 001277808.1 |
| 529 | | | Q9TSI0.1 |
| 530 | | | XP 006071186.1 |
| 531 | | | CAA06535.1 |
| 532 | | | 1004269A |
| 533 | | | ADD31643.1 |
| 534 | | | ADD31644.1 |
| 535 | | | AAT09469.1 |
| 536 | | | ABL10285.1 |
| 537 | | | ABA41625.1 |
| 538 | | | ABA41623.1 |
| 539 | Beta casein | Bos mutus | MXQ92033.1 |
| 540 | | | XP_014335713.1 |
| 541 | | | XP_005902099.2 |
| 542 | | | XP_014335715.1 |
| 543 | | | XP_014335714.1 |
| 544 | Beta casein | Bos indicus | AQY78354.1 |
| 545 | | | AQY78355.1 |
| 546 | | | ABL75279.1 |
| 547 | | | ABY27644.1 |
| 548 | | | AWN06759.1 |
| 549 | | | AGZ84117.1 |
| 550 | Beta casein | Bison bison bison | XP_010850446.1 |
| 551 | Beta casein (hypothetical | Jeotgalicoccus aerolatus | WP_188352529.1 |
| | protein) | | |
| 552 | Beta casein (hypothetical | Jeotgalicoccus coquinae | WP_188357544.1 |
| | protein) | | |
| 553 | Beta casein (precursor) | Bos indicus × Bos taurus | ARU83745.1 |
| 554 | | | AWN06757.1 |
| 555 | | | AWN06758.1 |
| 556 | Beta casein | Bos grunniens | AXE74295.1 |
| 557 | | | AEY63644.1 |
| 558 | | | AEY63645.1 |
| 559 | | | AEC13563.1 |
| 560 | Beta casein | Neophocaena asiaeorientalis | XP_024597374.1 |
| | | asiaeorientalis | |
| 561 | Beta casein | Odocoileus virginianus texanus | XP_020729180.1 |
| 562 | Beta casein (hypothetical | Muntiacus reevesi | KAB0354325.1 |
| | protein FD755_022863) | | |
| 563 | Beta casein (hypothetical | Muntiacus muntjak | KAB0345505.1 |
| | protein FD754_022431) | | |

Beta-Lactoglobulin sequences

| | | | |
|---|---|---|---|
| 9 | Optimized Beta | Artificial (codon optimized Bos | |
| | Lactoglobulin 1 (OLG1) | taurus) | |
| 10 | Optimized Beta | Bos taurus | |
| | Lactoglobulin 1 (OLG1) | | |
| 11 | Optimized Beta | Artificial (codon optimized Bos | |
| | Lactoglobulin 2 (OLG2) | taurus) | |
| 12 | Optimized Beta | Artificial (codon optimized Bos | |
| | Lactoglobulin 3 (OLG3) | taurus) | |
| 13 | Optimized Beta | Artificial (codon optimized Bos | |
| | Lactoglobulin 4 (OLG4) | taurus) | |
| 564 | Beta Lactoglobulin | Bos taurus | 5K06_A |
| 565 | | | 1B0O_A |
| 566 | | | NP_776354.2 |
| 567 | | | 3PH5_A |
| 568 | | | 1BEB_A |
| 569 | | | 6QPD_A |
| 570 | | | 6QI7_A |
| 571 | | | DAA24277.1 |
| 572 | | | 5HTD_A |
| 573 | | | 6QPE_A |
| 574 | | | 6RWR_A |
| 575 | | | 1BSO_A |
| 576 | | | 6RWQ_A |
| 577 | | | ACG59280.1 |
| 578 | | | 5NUJ_A |
| 579 | | | 5NUM_A |
| 580 | | | 1UZ2_X |
| 581 | | | CAA32835.1 |
| 582 | | | 1CJ5_A |
| 583 | | | 5NUK_A |
| 584 | | | 5NUN_A |
| 585 | | | 732164A |
| 586 | | | XP_024854027.1 |
| 587 | | | AAA30411.1 |
| 588 | Beta Lactoglobulin | Capra hircus | 4OMW_A |
| 589 | | | NP_001272468.1 |
| 590 | | | ABQ51182.1 |
| 591 | Beta Lactoglobulin | Ovis aries | 4NLI_A |
| 592 | | | NP_001009366.1 |
| 593 | | | 4CK4_A |
| 594 | | | 4CK4_B |
| 595 | Beta Lactoglobulin | Bubalus bubalis | 0601265A |
| 596 | | | P02755.2 |
| 597 | | | NP_001277893.1 |
| 598 | | | QOQ34530.1 |
| 599 | | | APQ30587.1 |
| 600 | | | ABG78270.1 |
| 601 | Beta Lactoglobulin | Bos mutus | XP_005888577.1 |
| 602 | | | MXQ94840.1 |
| 603 | Beta Lactoglobulin | Bos indicus | XP_019826641.1 |
| 604 | Beta Lactoglobulin | Jeotgalicoccus coquinae | WP_188357550.1 |
| | (lipocalin/fatty-acid | | |
| | binding family protein) | | |
| 605 | Beta Lactoglobulin | Jeotgalicoccus schoeneichii | WP_188349305.1 |
| | (lipocalin/fatty-acid | | |
| | binding family protein | | |
| 606 | Beta Lactoglobulin | Bison bison bison | XP_010855058.1 |
| 607 | Beta Lactoglobulin | Ovis sp. | AAA31510.1 |
| 608 | Beta Lactoglobulin | Ovis aries musimon | P67975.1 |
| 609 | Beta Lactoglobulin | Odocoileus virginianus texanus | XP_020744123.1 |
| 610 | Beta Lactoglobulin, | Rangifer tarandus | 1YUP_A |
| | Chain A | | |
| 611 | Beta Lactoglobulin | Rangifer tarandus tarandus | AAZ57420.1 |
| 612 | Beta Lactoglobulin | Muntiacus muntjak | KAB0364864.1 |
| | (hypothetical protein | | |
| | FD754 009020) | | |
| 613 | Beta Lactoglobulin | Muntiacus reevesi | KAB0379658.1 |
| | (hypothetical protein | | |
| | FD755 007442) | | |
| 614 | Beta Lactoglobulin, | Equus caballus | 3KZA_A |
| | Chain A | | |

### REFERENCES

Fox, P. F., and A. L. Kelly. “Chemistry and biochemistry of milk constituents.” Food Biochemistry and Food Processing 2 (2006): 442-464.
Garbarino, Joan E., and William R. Belknap. “Isolation of a ubiquitin-ribosomal protein gene (ubi3) from potato and expression of its promoter in transgenic plants.” Plant molecular biology 24, no. 1 (1994): 119-127.
Grey, Finn, Rebecca Tirabassi, Heather Meyers, Guanming Wu, Shannon McWeeney, Lauren Hook, and Jay A. Nelson. “A viral microRNA down-regulates multiple cell cycle genes through mRNA 5′ UTRs.” PLoS Pathog 6, no. 6 (2010): e1000967.
Laxa, Miriam. “Intron-mediated enhancement: a tool for heterologous gene expression in plants?.” Frontiers in plant science 7 (2017): Orom, Ulf Andersson, Finn Cilius Nielsen, and Anders H. Lund. “MicroRNA-10a binds the 5′ UTR of ribosomal protein mRNAs and enhances.
Ortega, Jose Luis, Olivia L. Wilson, and Champa Sengupta-Gopalan. “The 5′ untranslated region of the soybean cytosolic glutamine synthetase β 1 gene contains prokaryotic translation initiation signals and acts as a translational enhancer in plants.” Molecular genetics and genomics 287, no. 11 (2012): 881-893.
Strixner, T., & Kulozik, U. (2011). Egg proteins. In Handbook of food proteins (pp. 150-209). Woodhead publishing.
Tian, Li, and Samuel SM Sun. “Ubiquitin fusion expression and tissue-dependent targeting of hG-CSF in transgenic tobacco.” BMC biotechnology 11, no. 1 (2011): 91.
Tschofen, Marc, Dietmar Knopp, Elizabeth Hood, and Eva Stoger. “Plant molecular farming: much more than medicines.” Annual Review of Analytical Chemistry 9 (2016): 271-294.
Zou, Z., C. Eibl, and H-U. Koop. “The stem-loop region of the tobacco psbA 5′ UTR is an important determinant of mRNA stability and translation efficiency.” Molecular genetics and genomics 269, no. 3 (2003): 340-349.

### EMBODIMENTS

### Embodiment Set 1

1. A host cell that comprises an exogenous RNA sequence that encodes a chordate protein, wherein the exogenous RNA sequence is stabilized as determined by increased expression of the chordate protein as compared to an otherwise comparable host cell lacking the exogenous RNA sequence that is stabilized, and wherein the chordate protein is expressed in the amount of at least 1% or higher per total protein weight of soluble protein extractable from the host cell.
2. The host cell of embodiment 1, wherein the chordate is a vertebrate.
3. The host cell of embodiment 2, wherein the vertebrate is a mammal.
4. The host cell of embodiment 3, wherein the mammal is a bovine.
5. The host cell of embodiment 2, wherein the vertebrate is a bird.
6. The host cell of embodiment 5, wherein the bird is a chicken.
7. The host cell of any one of the preceding embodiments, wherein the chordate protein is an egg protein or a milk protein.
8. The host cell of embodiment 7, wherein the chordate protein is a milk protein.
9. The host cell of embodiment 8, wherein the milk protein is β-lactoglobulin.
10. The host cell of embodiment 7, wherein the chordate protein is an egg protein.
11. The host cell of embodiment 10, wherein the egg protein is ovalbumin.
12. The host cell of any one of the preceding embodiments, wherein the chordate protein is expressed in the amount of at least 2%, at least 3%, at least 4%, or at least 5% per total protein weight of soluble protein extractable from the host cell.
13. The host cell of any one of the preceding embodiments, wherein the chordate protein is expressed in the amount of about 1 to about 2%, about 2 to about 3%, or about 2 to about 5% per total protein weight of soluble protein extractable from the host cell.
14. A plant that comprises the host cell of any one of embodiments 1-13.
15. The plant of embodiment 14, wherein the plant is a soybean plant.
16. A DNA construct for expression of a transgene in a host cell, wherein the DNA construct comprises: a codon-optimized transgene sequence that comprises at least 90% identity to a sequence selected from the group consisting of SEQ ID NO: 1, 3, 5, 7, 9-13, 83, 617-621, 683-690, and 693-700; and an exogenous intron sequence, wherein the exogenous intron sequence comprises at least 90% identity to a sequence selected from the group consisting of: SEQ ID NO: 679-682.
17. The DNA construct of embodiment 16, wherein the codon-optimized transgene sequence comprises a sequence selected from SEQ ID NO: 1, 3, 5, 7, 9-13, 83, 617-621, 683-690, and 693-700.
18. The DNA construct of embodiment 16, wherein the exogenous intron sequence comprises a sequence selected from SEQ ID NO: 679-682.
19. A DNA construct for expression of a transgene in a host cell, wherein the DNA construct comprises: a codon-optimized transgene sequence that comprises at least 90% identity to a sequence selected from the group consisting of SEQ ID NO: 1, 3, 5, 7, 9-13, 83, 617-621, 683-690, and 693-700; and an exogenous intron sequence, wherein the exogenous intron sequence comprises at least 90% identity to a sequence selected from the group consisting of: SEQ ID NO: 679-682.
20. The DNA construct of embodiment 19, wherein the codon-optimized transgene sequence comprises a sequence selected from SEQ ID NO: 1, 3, 5, 7, 9-13, 83, 617-621, 683-690, and 693-700.
21. The DNA construct of embodiment 19, wherein the exogenous intron sequence comprises a sequence selected from SEQ ID NO: 679-682.
22. The DNA construct of any one of embodiments 16-21, wherein the DNA construct further comprises a signal peptide sequence.
23. The DNA construct of embodiment 22, wherein the signal peptide sequence is selected from the group consisting of: SEQ ID NO: 616, 707-717.
24. The DNA construct of any one of embodiments 16-23, wherein the DNA construct further comprises a sequence encoding a KDEL sequence.
25. The DNA construct of any one of embodiments 16-23, wherein the DNA construct further comprises a sequence encoding at least one of a 5′ UTR and a 3′ UTR.
26. The DNA construct of any one of embodiments 16-25, wherein the DNA construct further comprises a sequence encoding a ubiquitin monomer.
27. The DNA construct of any one of embodiments 16-26, wherein the DNA construct further comprises an exogenous promoter sequence.
28. The DNA construct of embodiment 27, wherein the exogenous promoter sequence is isolated or derived from a plant promoter sequence.
29. The DNA construct of embodiment 27, wherein the exogenous promoter sequence is isolated or derived from a seed promoter sequence.
30. The DNA construct of any one of embodiments 16-29, wherein the DNA construct further comprises an exogenous terminator sequence.
31. A composition that comprises the DNA construct of any one of embodiments 16-30.
32. A method of transforming a host cell, the method comprising contacting a host cell with the composition of embodiment 31, thereby transforming the host cell.
33. The method of embodiment 32, wherein the host cell is a plant cell.
34. The method of embodiment 33, wherein the method comprises bombardment or agrobacterium-mediated transformation.
35. The method of any one of embodiment 33-34, further comprising cultivating the plant cell after the transforming.
36. An RNA generated from the DNA construct of any one of embodiments 16-30.
37. A method of expressing ovalbumin or β-lactoglobulin in a plant, the method comprising: contacting at least a portion of a plant with the DNA construct of any one of embodiments 16-30, wherein the method is effective in increasing expression of the ovalbumin or β-lactoglobulin as compared to an otherwise comparable method lacking the contacting.
38. The method of embodiment 37, wherein the method is effective in increasing expression of the ovalbumin or β-lactoglobulin by at least about 1-fold as compared to an otherwise comparable method lacking the contacting.
39. A method of stably expressing a chordate protein in a plant cell, the method comprising: contacting a plant cell with a DNA construct that comprises at least 90% identity to a sequence selected from the group consisting of SEQ ID NO: 752-766, thereby generating a transformed plant cell; and cultivating a plant that comprises the transformed plant cell, thereby generating a transformed plant, wherein the chordate protein is expressed in the amount of 1% or higher per total protein weight of soluble protein extractable from the transformed plant cell.
40. The method of embodiment 39, wherein the DNA construct comprises at least 95%, at least 97%, or at least 99% identity to a sequence selected from the group consisting of SEQ ID NO: 752-766.
41. A method of stably expressing a chordate protein in a plant cell, the method comprising: contacting a plant cell with a DNA construct that comprises at least 90% identity to a sequence selected from the group consisting of SEQ ID NO: 767-781, thereby generating a transformed plant cell; and cultivating a plant that comprises the transformed plant cell, thereby generating a transformed plant, wherein the chordate protein is expressed in the amount of 1% or higher per total protein weight of soluble protein extractable from the transformed plant cell.
42. The method of embodiment 41, wherein the DNA construct comprises at least 95%, at least 97%, or at least 99% identity to a sequence selected from the group consisting of SEQ ID NO: 767-781.
43. The method of any one of embodiments 39-42, wherein the chordate protein is expressed in the amount of at least 1%, at least 2%, at least 3%, at least 4%, or at least 5% per total protein weight of soluble protein extractable from the transformed plant cell.
44. The method of any one of embodiments 39-43, wherein the plant cell is from a soybean plant.
45. The method of any one of embodiments 39-44, wherein the contacting comprises bombardment or agrobacterium-mediated transformation.
46. The method of any one of embodiments 39-45, wherein a level of a transcript of a transgene encoded by the DNA construct is increased by at least 1-fold as compared to an otherwise comparable method lacking the contacting.
47. The method of any one of embodiments 39-46, wherein a level of the chordate protein encoded by the DNA construct is increased by at least 1-fold as measured by ELISA and as compared to an otherwise comparable method lacking the contacting.
48. The method of embodiments 46 or 47, wherein the level is increased by at least 3-fold, at least 5-fold, at least 10-fold, at least 30-fold, or at least 50-fold.
49. The method of any one of embodiments 39-48, further comprising isolating a seed from the transformed plant.
50. A nutraceutical that comprises a chordate protein isolated from a transformed plant cell generated by the method of any one of embodiments 39-49.

### Embodiment Set 2

1. A method for selecting a nucleic acid sequence, said method comprising the steps of: a) providing data on a plurality of nucleic acid sequences; b) predicting secondary structure of the plurality of nucleic acid sequences, with a plurality of RNA folding models, such that each nucleic acid sequence in the plurality of nucleic acid sequences is associated with at least two predicted secondary structures; c) determining a structural similarity score for the at least two predicted secondary structures associated with each nucleic acid sequence; and d) selecting a nucleic acid sequence with a higher structural similarity score than at least one other nucleic acid sequence in the plurality of nucleic acid sequences; wherein the selected nucleic acid sequence is predicted to accumulate at higher levels when expressed in a host cell.
2. A method for selecting a nucleic acid sequence, said method comprising the steps of: a) providing data on a plurality of nucleic acid sequences, each nucleic acid sequence in the plurality of nucleic acid sequences being associated with at least two predicted secondary structures from different RNA folding models; b) determining a structural similarity score for the at least two predicted secondary structures associated with each nucleic acid sequence; d) selecting a nucleic acid sequence with a higher structural similarity score than at least one other nucleic acid sequence in the plurality of nucleic acid sequences; wherein the selected nucleic acid sequence is predicted to accumulate at higher levels when expressed in a host cell.
3. The method of embodiment 1 or 2, wherein at least one of the RNA folding models employs machine learning.
4. The method of embodiment 1 or 2, wherein the plurality of nucleic acid sequences encode the same amino acid sequence.
4.1 The method of embodiment 1 or 2, wherein the plurality of nucleic acid sequences encode amino acids sharing at least 90%, 91%, 92%, 93%, 94%, 95%, 96%, 97%, 98%, or 99% sequence identity.
5. The method of any one of embodiments 1-4, comprising: manufacturing the selected nucleic acid sequence into a nucleic acid.
6. The method of any one of embodiments 1-5, comprising: expressing the selected nucleic acid sequence in a host cell.
7. The method of embodiment 5, comprising expressing the manufactured nucleic acid in a host cell.
8. The method of any one of embodiments 1-6, wherein the nucleic acid sequence encodes for a messenger RNA.
9. The method of any one of embodiments 1-8, wherein the RNA folding models comprise a model selected from the group consisting of Cocke-Younger Kasami model, inside and outside models, loop-based energy model, minimum free energy, suboptimal folding, centroid, and any combination thereof.
9.1 The method of any one of embodiments 1-8, wherein the RNA folding models comprise a model selected from Tables 1 or 2.
10. The method of any one of embodiments 1-8, wherein the at least two predicted secondary structures are a minimum free energy structure and a centroid structure.
11. The method of any one of embodiments 1-10, wherein the structural similarity score is determined via tool selected from the group consisting of Consan, Dynalign, PMcomp, Stemloc, Foldalign, locARNA, SPARSE, MARNA, FoldAlignM, Murlet, CARNA, RAF, RNAforester, RNAdistance, RNAStrAt, RNApdist, and any combination thereof.
12. The method of any one of embodiments 1-10, wherein the structure similarity score is based on visual inspection of the predicted secondary structures.
13. The method of embodiment 12, wherein the structure similarity score is a ranking of the plurality of nucleic acid sequences based on the relative similarity of each nucleic acid sequences' predicted secondary structures.
14. The method of any one of embodiments 1-10, wherein the similarity score is based on degree of curve overlap in a graph depicting number of base pairs at each position of the predicted secondary structures.
15. The method of any one of embodiments 1-10, wherein the similarity score is based on the degree of curve overlap of the predicted secondary structures plotted in a mountain plot.
16. The method of any one of embodiments 1-10, wherein the similarity score is based on the correlation of curves representing the predicted secondary structures in a graph depicting number of base pairs at each position.
17. The method of embodiment 16, wherein the degree of curve overlap is calculated by methodology selected from the group consisting of least squares, curve length measure, and any combination thereof.
18. A method of manufacturing a nucleic acid, said method comprising: a) manufacturing a selected nucleic acid sequence to produce a nucleic acid, wherein the selection of the nucleic acid sequence was based on the selected nucleic acid sequence having a higher structural similarity score than at least one other nucleic acid sequence in a plurality of nucleic acid sequences; wherein the structural similarity score is based on the structural similarity between at least two predicted secondary structures for each nucleic acid sequence, the predicted secondary structures produced by different RNA folding models.
19. The method of embodiment 18, wherein at least one of the RNA folding models employs machine learning.
20. The method of embodiment 18 or 19, wherein the plurality of nucleic acid sequences encode the same amino acid sequence.
20.1 The method of embodiment 18 or 19, wherein the plurality of nucleic acid sequences encode amino acids sharing at least 90%, 91%, 92%, 93%, 94%, 95%, 96%, 97%, 98%, or 99% sequence identity.
21. The method of any one of embodiments 18-20, comprising: expressing the manufactured nucleic acid in a host cell.
22. The method of 21, wherein the manufactured nucleic acid expresses at a higher level than other nucleic acids containing other nucleic acid sequences from the plurality of nucleic acid sequences.
23. The method of any one of embodiments 18-22, wherein the RNA folding models comprise a model selected from the group consisting of Cocke-Younger Kasami model, inside and outside models, loop-based energy model, minimum free energy, suboptimal folding, centroid, and any combination thereof.
24. The method of any one of embodiments 18-22, wherein the at least two predicted secondary structures are a minimum free energy structure and a centroid structure.
25. The method of any one of embodiments 18-24, wherein the structural similarity score is determined via tool selected from the group consisting of Consan, Dynalign, PMcomp, Stemloc, Foldalign, locARNA, SPARSE, MARNA, FoldAlignM, Murlet, CARNA, RAF, RNAforester, RNAdistance, RNAStrAt, RNApdist, and any combination thereof.
25.1 The method of any one of embodiments 18-24, wherein the RNA folding models comprise a model selected from Tables 1 or 2.
26. The method of any one of embodiments 18-24, wherein the structure similarity score is based on visual inspection of the predicted secondary structures.
27. The method of embodiment 26, wherein the structure similarity score is a ranking of the plurality of nucleic acid sequences based on the relative similarity of each nucleic acid sequences' predicted secondary structures.
28. The method of any one of embodiments 18-24, wherein the similarity score is based on degree of curve overlap in a graph depicting number of base pairs at each position of the predicted secondary structures.
29. The method of any one of embodiments 18-24, wherein the similarity score is based on the degree of curve overlap of the predicted secondary structures plotted in a mountain plot.
30. A nucleic acid comprising the nucleic acid sequence selected in the method of any one of embodiments 1-29.
31. A host cell comprising a nucleic acid comprising a sequence of Table 11, Table 12, or Table 15.
32. The host cell of embodiment 31, wherein the nucleic acid comprises a sequence selected from the group consisting of: SEQ ID NO: 757, 760, 762, 763, 765, 772, 773, 778, and 780.
33. A host cell comprising a nucleic acid encoding any one of SEQ ID NO: 685, 687, and 695.
34. An automated system for predicting relative expression strength of a plurality of nucleic acid sequences expression in vivo, the system comprising: i) a memory; and ii) a processor in communication with the memory, the processor configured to: a) define a plurality of nucleic acid sequences; b) predict secondary structure of the plurality of nucleic acid sequences, with a plurality of RNA folding models, such that each nucleic acid sequence in the plurality of nucleic acid sequences is associated with at least two predicted secondary structures; c) determine a structural similarity score for the at least two predicted secondary structures associated with each nucleic acid sequence; wherein nucleic acid sequences with similarity scores indicative of greater structure similarity are predicted to accumulate at higher levels than nucleic acid sequences with scores indicative of lower structural similarity, when expressed in a host cell.
35. The system of embodiment 34, wherein at least one of the RNA folding models employs machine learning.
36. The system of embodiment 34 or 35, wherein the plurality of nucleic acid sequences encode the same amino acid sequence.
37. The system of embodiment 34 or 35, wherein the plurality of nucleic acid sequences encode amino acids sharing at least 90%, 91%, 92%, 93%, 94%, 95%, 96%, 97%, 98%, or 99% sequence identity.
38. The system of any one of embodiments 34-37, wherein the processor is configured to manufacture a nucleic acid sequence from the plurality of nucleic acid sequences, into a nucleic acid.
39. The system of any one of embodiments 34-37, wherein the processor is configured to send instructions to automated liquid and particle handling robotics to cause the automated liquid and particle handling robotics to manipulate liquid or particles added to or removed from reactions to manufacture a nucleic acid with the nucleic acid sequence from the plurality of nucleic acid sequences.
40. The system of any one of embodiments 34-39, wherein the processor is configured to express a nucleic acid sequence from the plurality of nucleic acid sequences, in a host cell.
41. The system of embodiment 40, wherein the nucleic acid sequence expressed is the nucleic acid manufactured in embodiment 39.
42. The system of any one of embodiments 34-39, wherein the processor is configured to send instructions to automated liquid and particle handling robotics to cause the automated liquid and particle handling robotics to manipulate liquid or particles added to or removed from cultures having a base host cells to create an engineered host cell expressing a nucleic acid sequence from the plurality of nucleic acid sequences.
43. The system of any one of embodiments 34-42, wherein the nucleic acid sequence encodes for a messenger RNA.
44. The system of any one of embodiments 34-43, wherein the wherein the processor is configured to select a nucleic acid sequence from the plurality of nucleic acid sequences that is predicted to accumulate at 10%, 20%, 30%, 40%, 50% or more higher levels than at least on other nucleic acid in the plurality of nucleic acid sequences.
45. The system of any one of embodiments 34-44, wherein the RNA folding models comprise a model selected from the group consisting of Cocke-Younger Kasami model, inside and outside models, loop-based energy model, minimum free energy, suboptimal folding, centroid, and any combination thereof.
46. The system of any one of embodiments 34-44, wherein the at least two predicted secondary structures are a minimum free energy structure and a centroid structure.
47. The system of any one of embodiments 34-46, wherein the structural similarity score is determined via tool selected from the group consisting of Consan, Dynalign, PMcomp, Stemloc, Foldalign, locARNA, SPARSE, MARNA, FoldAlignM, Murlet, CARNA, RAF, RNAforester, RNAdistance, RNAStrAt, RNApdist, and any combination thereof.
47.1 The system of any one of embodiments 34-46, wherein the RNA folding models comprise a model selected from Tables 1 or 2.
48. A composition comprising or consisting of a sequence with at least about 80%, 85%, 90%, 95%, 96%, 97%, 98%, 99%, or 100% identity to any one of SEQ ID NO: 829-831.
49. A nucleic acid comprising or consisting of a sequence with at least about 80%, 85%, 90%, 95%, 96%, 97%, 98%, 99%, or 100% identity to any one of SEQ ID NO: 829-831.
50. A polypeptide encoded by a sequence with at least about 80%, 85%, 90%, 95%, 96%, 97%, 98%, 99%, or 100% identity to any one of SEQ ID NO: 829-831.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

1. A method for selecting a nucleic acid sequence, said method comprising the steps of:
a) providing data on a plurality of nucleic acid sequences;
b) predicting secondary structure of the plurality of nucleic acid sequences with a plurality of RNA folding models, such that each nucleic acid sequence in the plurality of nucleic acid sequences is associated with at least two predicted secondary structures;
c) determining a structural similarity score for the at least two predicted secondary structures associated with each nucleic acid sequence; and
d) selecting a nucleic acid sequence with a higher structural similarity score than at least one other nucleic acid sequence in the plurality of nucleic acid sequences; wherein the selected nucleic acid sequence is predicted to accumulate at higher levels when expressed in a host cell.

2. The method of claim 1, wherein at least one of the plurality of RNA folding models employs machine learning.

3. The method of claim 1, wherein the plurality of nucleic acid sequences encode the same amino acid sequence.

4. The method of claim 1, wherein the plurality of nucleic acid sequences encode amino acids sharing at least 95% sequence identity.

5. The method of claim 1, comprising: manufacturing the selected nucleic acid sequence into a nucleic acid.

6. The method of claim 1, comprising: expressing the selected nucleic acid sequence in a host cell.

7. The method of claim 5, comprising expressing the manufactured nucleic acid in a host cell.

8. The method of claim 1, wherein the nucleic acid sequence encodes for a messenger RNA.

9. The method of claim 1, wherein the plurality of RNA folding models comprise a model selected from the group consisting of Cocke-Younger Kasami model, inside and outside models, loop-based energy model, minimum free energy, suboptimal folding, centroid, and any combination thereof.

10. The method of claim 1, wherein the at least two predicted secondary structures are a minimum free energy structure and a centroid structure.

11. The method of claim 1, wherein the structural similarity score is determined via tool selected from the group consisting of Consan, Dynalign, PMcomp, Stemloc, Foldalign, locARNA, SPARSE, MARNA, FoldAlignM, Murlet, CARNA, RAF, RNAforester, RNAdistance, RNAStrAt, RNApdist, and any combination thereof.

12. The method of claim 1, wherein the structural similarity score is a ranking of the plurality of nucleic acid sequences based on the relative similarity of each nucleic acid sequences' predicted secondary structures.

13. The method of claim 1, wherein the structural similarity score is based on degree of curve overlap in a graph depicting number of base pairs at each position of the predicted secondary structures of each nucleic acid sequence.

14. The method of claim 1, wherein the structure similarity score is based on the degree of curve overlap of the predicted secondary structures of each nucleic acid sequence, plotted in a mountain plot.

15. The method of claim 1, wherein the structural similarity score is based on the correlation of curves representing the predicted secondary structures for each nucleic acid sequence in a graph depicting number of base pairs at each position.

16. The method of claim 13, wherein the degree of curve overlap is calculated by methodology selected from the group consisting of least squares, curve length measure, and any combination thereof.

17. A method of manufacturing a nucleic acid, said method comprising:
a) manufacturing a selected nucleic acid sequence to produce a nucleic acid,
wherein the selection of the nucleic acid sequence was based on the selected nucleic acid sequence having a higher structural similarity score than at least one other nucleic acid sequence in a plurality of nucleic acid sequences;
wherein the structural similarity score is based on the structural similarity between at least two predicted secondary structures for each nucleic acid sequence, the at least two predicted secondary structures produced by different RNA folding models.

18. The method of claim 17, wherein at least one of the RNA folding models employs machine learning.

19. The method of claim 17, wherein the plurality of nucleic acid sequences encode the same amino acid sequence.

20. The method of claim 17, wherein the plurality of nucleic acid sequences encode amino acids sharing at least 95% sequence identity.


SEQUENCE LISTING (831 sequences)

SEQ ID NO: 1 - DNA, 318 bp, synthetic construct
  misc_feature 1..318: note = Optimized para-kappa-casein truncated version 1 (paraOKC1-T)
  source 1..318: mol_type = other DNA
caagagcagaatcaagagcagccaatccgttgtgagaaggacgagaggttcttctcagacaagatcgccaaatatatacccatacaatatgtactctcacgctaccctagctacgggcttaactactatcagcaaaaacctgtagcactgataaataaccagtttctcccctatccctattatgctaaacctgccgccgtgaggagtccagcacaaatacttcagtggcaagtgctcagtaacaccgtgccagcaaaaagctgccaggctcagcccaccacaatggcccgtcatccccatcctcaccttagcttcatg

SEQ ID NO: 2 - protein, 106 aa, synthetic construct
  REGION 1..106: note = Optimized para-kappa-casein truncated version 1 (paraOKC1-T)
QEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFM

SEQ ID NO: 3 - DNA, 507 bp, synthetic construct
  misc_feature 1..507: note = Optimized kappa-casein truncated version 1 (OKC1-T)
  source 1..507: mol_type = other DNA
caagagcagaatcaagagcagccaatccgttgtgagaaggacgagaggttcttctcagacaagatcgccaaatatatacccatacaatatgtactctcacgctaccctagctacgggcttaactactatcagcaaaaacctgtagcactgataaataaccagtttctcccctatccctattatgctaaacctgccgccgtgaggagtccagcacaaatacttcagtggcaagtgctcagtaacaccgtgccagcaaaaagctgccaggctcagcccaccacaatggcccgtcatccccatcctcaccttagcttcatggcaatcccaccaaagaagaatcaagacaagaccgaaatacctaccatcaacacaattgcatctggagagcctaccagtacaccaacaactgaggcagtagagtctactgttgctacccttgaggacagccccgaggttatagagtccccacctgagataaataccgtgcaggtgacaagtaccgccgta

SEQ ID NO: 4 - protein, 169 aa, synthetic construct
  REGION 1..169: note = Optimized kappa-casein truncated version 1 (OKC1-T)
QEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEDSPEVIESPPEINTVQVTSTAV

SEQ ID NO: 5 - DNA, 627 bp, synthetic construct
  misc_feature 1..627: note = Optimized beta-casein truncated version 2 (OBC-T2)
  source 1..627: mol_type = other DNA
cgcgaactggaagagttgaacgtaccaggagagattgtagaatcactgagctcctcagaggagtctattactcgtatcaacaagaagatagagaagttccaatccgaggagcaacaacaaacagaggacgaattgcaggacaagatacatcctttcgcacagacccagagcctcgtctatccctttccaggtccaatccctaactctctcccccagaatatcccacccttgactcagactcccgtggtcgtaccccctttcttgcaacccgaggtgatgggggtttctaaagtcaaagaggctatggctcctaaacataaggaaatgccttttcccaaatatccagtggagccattcactgagagccagtctctgacacttacagatgtggaaaacttgcacctgcccttgccacttttgcagtcctggatgcaccaaccacatcaacccttgccccccacagtgatgtttcctccacaatcagttcttagtctctcccaaagcaaagtccttccagtgcctcagaaggccgtcccatacccccagagagatatgccaatacaggcattcttgctttaccaggaaccagtgctcggtcctgtacgtggcccattccctatcatagtg

SEQ ID NO: 6 - protein, 209 aa, synthetic construct
  REGION 1..209: note = Optimized beta-casein truncated version 2 (OBC-T2)
RELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 7 - DNA, 597 bp, synthetic construct
  misc_feature 1..597: note = Optimized alpha S1-casein truncated version 1(OaS1-T)
  source 1..597: mol_type = other DNA
cgcccaaaacatcccataaaacatcaaggattgccccaggaagtactcaacgagaatctcctccgttttttcgttgctcctttccccgaagtgttcgggaaggaaaaagtaaacgagctttcaaaggacatcggctctgaaagtaccgaggatcaggctatggaagatatcaagcaaatggaggccgaatctataagttcttcagaagaaatagttcccaactcagtggagcagaagcacattcagaaagaagacgtgcccagcgagcgctatctgggatatttggaacagctgctcagactgaaaaagtacaaggtgcctcagctcgaaatcgtacccaatagtgctgaagaaaggttgcactcaatgaaagaggggattcacgcacaacaaaaagagcctatgatcggagtaaatcaagaactggcatacttttatcccgagttgtttcgccaattctatcaactggatgcctacccttccggtgcatggtactacgtacccctcggtactcaatataccgatgctccctccttttccgacattcctaatcctataggttccgagaatagcgaaaagaccaccatgcccttatgg

SEQ ID NO: 8 - protein, 199 aa, synthetic construct
  REGION 1..199: note = Optimized alpha S1-casein truncated version 1(OaS1-T)
RPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 9 - DNA, 486 bp, synthetic construct
  misc_feature 1..486: note = Optimized Beta Lactoglobulin 1 (OLG1)
  source 1..486: mol_type = other DNA
ttgatcgtaacacagactatgaagggtcttgatatacagaaggtggccgggacttggtacagtttggcaatggccgcatccgacatctccttgttggacgcacaatcagccccattgcgtgtgtacgtagaagagcttaaaccaactcccgagggggatctggaaattctgctccagaaatgggagaacggtgagtgcgcccagaagaagatcatcgcagagaagaccaaaattccagcagtattcaaaatcgacgcattgaacgaaaataaggtgctcgtactggacactgattataagaagtatctccttttctgtatggagaactcagcagagcctgaacagagtcttgcctgccaatgccttgttcgtaccccagaggtagatgatgaagctctggaaaagttcgataaggcccttaaggctctgcctatgcacattaggctttctttcaatccaactcaacttgaggaacaatgtcacatt

SEQ ID NO: 10 - protein, 162 aa, synthetic construct
  REGION 1..162: note = Optimized Beta Lactoglobulin 1 (OLG1)
LIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHI

SEQ ID NO: 11 - DNA, 486 bp, synthetic construct
  misc_feature 1..486: note = Optimized Beta Lactoglobulin 2 (OLG2)
  source 1..486: mol_type = other DNA
cttattgtgacccaaaccatgaagggcctcgacattcaaaaggttgccggaacctggtactcccttgctatggctgcttccgatatctccttgctcgatgctcaatccgctccacttagggtgtacgtggaagagttgaagccaactccagagggcgatcttgagatcttgcttcaaaagtgggagaacgatgagtgcgcccagaagaagattatcgccgaaaagaccaagattcccgccgtgttcaagatcgatgctctcaacgagaacaaggtgctcgtgctcgataccgactacaagaagtaccttctcgtctgcatggaaaactccgctgagccagagcaatctcttgtttgccaatgccttgtgaggaccccagaggttgacgatgaagctcttgagaagttcgacaaggctctcaaggctttgcctatgcacatccgccttagcttcaacccaactcagcttgaggaacagtgccacatc

SEQ ID NO: 12 - DNA, 486 bp, synthetic construct
  misc_feature 1..486: note = Optimized Beta Lactoglobulin 3 (OLG3)
  source 1..486: mol_type = other DNA
ctcattgttacacaaaccatgaagggtcttgacattcagaaggttgctgggacatggtattcactagcgatggctgcttctgatatctccctgttggatgcacagtctgcccccctgagagtgtatgttgaagaactgaaaccgacacctgaaggagacttggaaattttactccagaaatgggaaaatgatgagtgtgcccaaaagaagataatagccgagaagaccaaaattcctgctgtgtttaagattgatgctttgaatgagaacaaagtactagtcctcgacactgattacaagaaatacttattagtgtgcatggaaaacagcgcagagccagaacaatcacttgtttgtcaatgtttggtccgtactccagaggtagatgatgaagcattggagaaatttgataaagcattgaaggcacttccaatgcatataaggcttagtttcaatcctactcagcttgaagagcaatgccacatc

SEQ ID NO: 13 - DNA, 486 bp, synthetic construct
  misc_feature 1..486: note = Optimized Beta Lactoglobulin 4 (OLG4)
  source 1..486: mol_type = other DNA
cttatagtaactcaaaccatgaagggacttgatatccaaaaagttgcaggaacctggtactcactggctatggcagcttccgacatctccttgttggacgcacaatccgcaccattgcgcgtctacgttgaggagttgaaacctacaccagagggggatcttgagattttgctccagaaatgggagaacgacgagtgtgcccagaaaaaaattatagcagagaagactaaaattcctgctgtttttaagattgatgccctgaacgagaataaggtactggtcctcgacactgattataaaaagtatttgctggtgtgtatggagaacagtgctgaacctgaacagagcctggtctgtcaatgtcttgtaaggacacctgaggttgatgacgaggcacttgaaaaattcgacaaggcccttaaggctctgcctatgcacatccgtctgagtttcaaccctactcagttggaggaacaatgtcatatt

SEQ ID NO: 14-82 - skipped (69 entries)

SEQ ID NO: 83 - protein, 621 aa, synthetic construct
  REGION 1..621: note = Optimized alpha S2-casein truncated version 1(OaS2-T)
AAGAATACTATGGAACACGTAAGCTCAAGTGAAGAATCTATAATAAGTCAAGAGACATATAAGCAAGAGAAAAACATGGCAATAAATCCCTCCAAGGAGAATCTTTGTAGCACTTTTTGCAAAGAAGTTGTGAGAAATGCAAATGAGGAAGAATACTCAATAGGCAGCTCTTCCGAAGAATCTGCTGAAGTCGCTACTGAAGAGGTCAAAATAACAGTTGACGACAAGCATTATCAAAAAGCCCTGAATGAAATAAACCAGTTCTACCAAAAATTTCCCCAATACCTCCAGTACCTTTATCAAGGACCCATAGTCCTCAACCCTTGGGATCAGGTCAAGCGTAATGCTGTTCCAATAACACCAACACTCAATCGTGAACAACTGTCTACCTCAGAAGAAAATTCCAAAAAAACTGTGGATATGGAAAGTACAGAAGTTTTTACTAAAAAGACCAAGCTCACCGAGGAGGAAAAAAATAGATTGAATTTTCTTAAGAAGATCAGTCAACGCTATCAGAAGTTCGCCCTTCCACAATACCTCAAGACTGTATACCAACATCAGAAGGCCATGAAGCCTTGGATTCAGCCCAAAACAAAGGTAATCCCCTATGTTAGATACTTG

SEQ ID NO: 84 - protein, 207 aa, synthetic construct
  REGION 1..207: note = Optimized alpha S2-casein truncated version 1(OaS2-T)
KNTMEHVSSSEESIISQETYKQEKNMAINPSKENLCSTFCKEVVRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMKPWIQPKTKVIPYVRYL

SEQ ID NO: 85 - protein, 171 aa, Capra hircus
QEQNQEQPICCEKDERFFDDKIAKYIPIQYVLSRYPSYGLNYYQQRPVALINNQFLPYPYYAKPVAVRSPAQTLQWQVLPNTVPAKSCQDQPTTLARHPHPHLSFMAIPPKKDQDKTEVPAINTIASAEPTVHSTPTTEAIVNTVDNPEASSESIASASETNTAQVTSTEV

SEQ ID NO: 86 - protein, 171 aa, Ovis aries
QEQNQEQRICCEKDERFFDDKIAKYIPIQYVLSRYPSYGLNYYQQRPVALINNQFLPYPYYAKPVAVRSPAQTLQWQVLPNAVPAKSCQDQPTAMARHPHPHLSFMAIPPKKDQDKTEIPAINTIASAEPTVHSTPTTEAVVNAVDNPEASSESIASAPETNTAQVTSTEV

SEQ ID NO: 87 - protein, 165 aa, Bubalus bubalis
QEQNQEQPIRCEKEERFFNDKIAKYIPIQYVLSRYPSYGLNYYQQKPVALINNQFLPYPYYAKPAAVRSPAQILQWQVLPNTVPAKSCQAQPTTMTRHPHPHLSFMAIPPKKNQDKTEIPTINTIVSVEPTSTPTTEAIENTVATLEASSEVIESVPETNTAQVT

SEQ ID NO: 88 - protein, 162 aa, Camelus dromedarius
EVQNQEQPTCFEKVERLLNEKTVKYFPIQFVQSRYPSYGINYYQHRLAVPINNQFIPYPNYAKPVAIRLHAQIPQCQALPNIDPPTVERRPRPRPSFIAIPPKKTQDKTVNPAINTVATVEPPVIPTAEPAVNTVVIAEASSEFITTSTPETTTVQITSTEI

SEQ ID NO: 89 - protein, 162 aa, Camelus bactrianus
EVQNQEQPTCCEKVERLLNEKTVKYFPIQFVQSRYPSYGINYYQHRLAVPINNQFIPYPNYAKPVAIRLHAQIPQCQALPNIDPPTVERRPRPRPSFIAIPPKKTQDKTVNPAINTVATVEPPVIPTAEPAVNTVVIAEASSEFITTSTPETTTVQITSTEI

SEQ ID NO: 90 - protein, 173 aa, Bos mutus
QEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEASPEASPEVIESPPEINTVQVTSTAV

SEQ ID NO: 91 - protein, 165 aa, Equus caballus
EVQNQEQPTCHKNDERFFDLKTVKYIPIYYVLNSSPRYEPIYYQHRLALLINNQHMPYQYYARPAAVRPHVQIPQWQVLPNIYPSTVVRHPCPHPSFIAIPPKKLQEITVIPKINTIATVEPTPIPTPEPTVNNAVIPDASSEFIIASTPETTTVPVTSPVVQKL

SEQ ID NO: 92 - protein, 162 aa, Equus asinus
EVQNQEQPTCRKNDERFFDLKTVKYIPIYYVLNSSPRNEPIYYQHRLAVLINNQHMPYQYYARPAAVRPHVQIPQWQVLPNIYPSTVVRHPRPHPSFIAIPPKKLQEKTVIPKINTIATVEPTPIPTPEPTVNNAVIPDASSEFIIASTPETTTVPVTSPVV

SEQ ID NO: 93 - protein, 122 aa, Rangifer tarandus
VALINNQFLPYPYYAKPGAVRSPAQILQWQVLPNTVPAKSCQAQPTTLARHPHPRLSFMAIPPKKNQDKTDIPTINTIATVESTITPTTEAIVDTVATLEASSEVIESAPETNTDQVTSTVV

SEQ ID NO: 94 - protein, 141 aa, Alces alces
KIVKYIPIQYALSRYPSYGLSYYQHRPVALINNQFLPYPYYAKPGAVRSPAQILQWQVLPNTVPAKSCQAQPTTMARHPRPRLSFMAIPPKKNQDKTDIPTINTIATVESTITPTTEAIEDNVATLEASSEVIESAPETNT

SEQ ID NO: 95 - protein, 162 aa, Vicugna pacos
EVQNQEQPTCCEKVERLLNEKTVKYFPIQFVQSRYPSYGINYYQHRLAVPINNQFIPYPNYAKPVAIRLHAQIPQCQALPNIDPPTVERRPRPRPSFIAIPPKKTQDKTVIPAINTVATAEPPVIPTAEPVVNTVVIAEASSEFITTSTPETTTVQITSTEI

SEQ ID NO: 96 - protein, 160 aa, Bos indicus
RCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEDSPEVIESPPEINTVQVTSTAV

SEQ ID NO: 97 - protein, 162 aa, Lama glama
EVQNQEQPTCCEKVERLLNEKTVKYFPIQFVQSRYPSYGINYYQHRLAVPINNQFIPYPNYAKPVAIRLHAQIPQCQALPNIDPPTVERRPRPRPSFIAIPPKKTQDKTVIPAINTVATVEPPVIPTAEPVVNTVVIAEASSEFITTSTPETTTVQITSTEI

SEQ ID NO: 98 - protein, 162 aa, Homo sapiens
EVQNQKQPACHENDERPFYQKTAPYVPMYYVPNSYPYYGTNLYQRRPAIAINNPYVPRTYYANPAVVRPHAQIPQRQYLPNSHPPTVVRRPNLHPSFIAIPPKKIQDKIIIPTINTIATVEPTPAPATEPTVDSVVTPEAFSESIITSTPETTTVAVTPPTA

SEQ ID NO: 99 - protein, 199 aa, Capra hircus
RPKHPINHRGLSPEVPNENLLRFVVAPFPEVFRKENINELSKDIGSESTEDQAMEDAKQMKAGSSSSSEEIVPNSAEQKYIQKEDVPSERYLGYLEQLLRLKKYNVPQLEIVPKSAEEQLHSMKEGNPAHQKQPMIAVNQELAYFYPQLFRQFYQLDAYPSGAWYYLPLGTQYTDAPSFSDIPNPIGSENSGKTTMPLW

SEQ ID NO: 100 - protein, 199 aa, Ovis aries
RPKHPIKHQGLSSEVLNENLLRFVVAPFPEVFRKENINELSKDIGSESIEDQAMEDAKQMKAGSSSSSEEIVPNSAEQKYIQKEDVPSERYLGYLEQLLRLKKYNVPQLEIVPKSAEEQLHSMKEGNPAHQKQPMIAVNQELAYFYPQLFRQFYQLDAYPSGAWYYLPLGTQYTDAPSFSDIPNPIGSENSGKITMPLW

SEQ ID NO: 101 - protein, 199 aa, Bubalus bubalis
RPKQPIKHQGLPQGVLNENLLRFFVAPFPEVFGKEKVNELSTDIGSESTEDQAMEDIKQMEAESISSSEEIVPISVEQKHIQKEDVPSERYLGYLEQLLRLKKYNVPQLEIVPNLAEEQLHSMKEGIHAQQKEPMIGVNQELAYFYPQLFRQFYQLDAYPSGAWYYVPLGTQYPDAPSFSDIPNPIGSENSGKTTMPLW

SEQ ID NO: 102 - protein, 154 aa, Camelus dromedarius
DTERKESGSSSSEEVVSSTTEQKDILKEDMPSQRYLEELHRLNKYKLLQLEAIRDQKLIPRVKLSSHPYLEQLYRINEDNHPQLGEPVKVVTQEQAYFHLEPFPQFFQLGASPYVAWYYPPQVMQYIAHPSSYDTPEGIASEDGGKTDVMPQWW

SEQ ID NO: 103 - protein, 207 aa, Camelus bactrianus
RPKYPLRYPEVFQNEPDSIEEVLNKRKILELAVVSPIQFRQENIDELKDTRNEPTEDHIMEDTERKESGSSSSEEVVSSTTEQKDILKEDMPSQRYLEELHRLNKYKLLQLEAIRDQKLIPRVKLSSHPYLEQLYRINEDNHPQLGEPVKVVTQPFPQFFQLGASPYVAWYYPPQVMQYIAHPSSYDTPEGIASEDGGKTDVMPQWW

SEQ ID NO: 104 - protein, 199 aa, Bos mutus
RPKHPIKHQGLPQEVLNENLLRFFVAPFPEVFGKEKVNELSKDIGSESTEDQAMEDIKQMEAESISSSEEIVPNSVEQKHIQKEDVPSEHYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSGKTTMPLW

SEQ ID NO: 105 - protein, 226 aa, Equus caballus
REKEELNVSSETVESLSSNEPDSSSEESITHINKEKLQKFKHEGQQQREVERQDKISRFVQPQPVVYPYAEPVPYAVVPQSILPLAQPPILPFLQPEIMEVSQAKETILPKRKVMPFLKSPIVPFSERQILNPTNGENLRLPVHLIQPFMHQVPQSLLQTLMLPSQPVLSPPQSKVAPFPQPVVPYPQRDTPVQAFLLYQDPRLGPTGELDPATQPIVAVHNPVIV

SEQ ID NO: 106 - protein, 202 aa, Equus asinus
RPKLPHRHPEIIQNEQDSREKVLKERKFPSFALHTPREEYINELNRQRELLKEKQKDEHKEYLIEDPEQQESSSTSSSEEVVPINTEQKRIPREDMLYQHTLEQLRRLSKYNQLQLQAIYAQEQLIRMKENSQRKPMRVVNQEQAYFYLEPFQPSYQLDVYPYAAWFHPAQIMQHVAYSPFHDTAKLIASENSEKTDIIPEW

SEQ ID NO: 107 - protein, 199 aa, Bos indicus
  VARIANT 84: note = X is any amino acid
RPKHPIKHQGLPQEVLNENLLRFFVAPFPEVFGKEKVNELSKDIGSESTEDQAMEDIKQMEAESISSSEEIVPNSVEQKHIQKXDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSGKTTMPLW

SEQ ID NO: 108 - protein, 215 aa, Lama glama
RPKYPLRYPEVFQNEPDSIQEVLNKRKILELAVVSPIQFRQENIDELKDTRNEPTEDHIMEDTERTVSGSSSSEEVVSSTTEQKDILKEDMPSQRILEELHRLNKYKLLQLEAIRDQKLIPRVKLSSHPYLEQLYRINEDNHPQLGEPVKVVTQEQAYFHLEPFQQFFQLGASPYVAWYYPPQVMQYIAHPSSHDTPEGIASEDGGKTDVMPQWW

SEQ ID NO: 109 - protein, 170 aa, Homo sapiens
RPKLPLRYPERLQNPSESSEPIPLESREEYMNGMNRQRNILREKQTDEIKDTRNESTQNCVVAEPEKMESSISSSSEEMSLSKCAEQFCRLNEYNQLQLQAAHAQEQIRRMNENSHVQVPFQQLNQLAAYPYAVWYYPQIMQYVPFPPFSDISNPTAHENYEKNNVMLQW

SEQ ID NO: 110 - protein, 208 aa, Capra hircus
KHKMEHVSSSEEPINIFQEIYKQEKNMAIHPRKEKLCTTSCEEVVRNANEEEYSIRSSSEESAEVAPEEIKITVDDKHYQKALNEINQFYQKFPQYLQYPYQGPIVLNPWDQVKRNAGPFTPTVNREQLSTSEENSKKTIDMESTEVFTKKTKLTEEEKNRLNFLKKISQYYQKFAWPQYLKTVDQHQKAMKPWTQPKTNAIPYVRYL

SEQ ID NO: 111 - protein, 208 aa, Ovis aries
KHKMEHVSSSEEPINISQEIYKQEKNMAIHPRKEKLCTTSCEEVVRNADEEEYSIRSSSEESAEVAPEEVKITVDDKHYQKALNEINQFYQKFPQYLQYLYQGPIVLNPWDQVKRNAGPFTPTVNREQLSTSEENSKKTIDMESTEVFTKKTKLTEEEKNRLNFLKKISQYYQKFAWPQYLKTVDQHQKAMKPWTQPKTNAIPYVRYL

SEQ ID NO: 112 - protein, 207 aa, Bubalus bubalis
KHTMEHVSSSEESIISQETYKQEKNMAIHPSKENLCSTFCKEVIRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVITKKTKLTEEDKNRLNFLKKISQHYQKFTWPQYLKTVYQYQKAMKPWTQPKTNVIPYVRYL

SEQ ID NO: 113 - protein, 178 aa, Camelus dromedarius
KHEMDQGSSSEESINVSQQKFKQVKKVAIHPSKEDICSTFCEEAVRNIKEVESAEVPTENKISQFYQKWKFLQYLQALHQGQIVMNPWDQGKTRAYPFIPTVNTEQLSISEESTEVPTEESTEVFTKKTELTEEEKDHQKFLNKIYQYYQTFLWPEYLKTVYQYQKTMTPWNHIKRYF

SEQ ID NO: 114 - protein, 178 aa, Camelus bactrianus
KHEMDQGSSSEESINVSQQKFKQVKKVAIHPSKEDICSTFCEEAVRNIKEVESAEVPTENKISQFYQKWKFLQYLQALHQGQIVMNPWDQGKTRAYPFIPTVNTEQLSISEESTEVPTEESTEVFNKKTELTEEEKDHQKFLNKIYQYYQTFLWPEYLKTVYQYQKTMTPWNHIKRYF

SEQ ID NO: 115 - protein, 204 aa, Bos mutus
KNTMEHVSSSEESIISQETYKQEKNMAINPSKGNLCSTFCKEVVRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMKPWIQPKTKVIPYV

SEQ ID NO: 116 - protein, 216 aa, Equus caballus
KHNMEHRSSSEDSVNISQEKFKQEKYVVIPTSKESICSTSCEEATRNINEMESAKFPTEVYSSSSSSEESAKFPTEREEKEVEEKHHLKQLNKINQFYEKLNFLQYLQALRQPRIVLTPWDQTKTGDSPFIPIVNTEQLFTSEEIPKKTVDMESTEVVTEKTELTEEEKNYLKLLYYEKFTLPQYFKIVRQHQTTMDPRSHRKTNSYQIIPVLRYF

SEQ ID NO: 117 - protein, 221 aa, Equus asinus
KHNMEHRSSSEDSVNISQEKFKQEKYVVIPTSKESICSTSCEEATRNINEMESAKFPTEVYSSSSSSEESAKFPTEREEKEVEEKHHLKQLNKINQFYEKLNFLQYLQALRQPRIVLTPWDQTKTGASPFIPIVNTEQLFTSEEIPKKTVDMESTEVVTEKTELTEEEKNYLKLLNKINQYYEKFTLPQYFKIVHQHQTTMDPQSHSKTNSYQIIPVLRYF

SEQ ID NO: 118 - protein, 192 aa, Vicugna pacos
KHEMDQGSSSEESINVSQQKLKQVKKVAIHPSKEDICSTFCEEAVRNIKEVESVEVPTENKISQFYQKWKFLQYLQALHQGQIVMNPWDQGKTMVYPFIPTVNTEQLSISEESTEVPTEESTEVFTKKTELTEEEKDHQKFLNKIYQYYQTFLWPEYLKTVYQYQKTMTPWNHIKVKAYQIIPNLVSSTFYL

SEQ ID NO: 119 - protein, 207 aa, Bos indicus; identical to SEQ ID NO: 84

SEQ ID NO: 120 - protein, 187 aa, Lama glama
KHEMDQGSSSEESINVSQQKLKQVKKVAIHPSKEDICSTFCEEAVRNIKEVESVEVPTENKISQFYQKWKFLQYLQALHQGQIVMNPWDQGKTMVYPFIPTVNTEQLSISEESTEVPTEENSKKTVDTESTEVFTKKTELTEEEKDHQKFLNKIYQYYQTFLWPEYLKTVYQYQKTMTPWNHIKRYF

SEQ ID NO: 121 - protein, 207 aa, Capra hircus
REQEELNVVGETVESLSSSEESITHINKKIEKFQSEEQQQTEDELQDKIHPFAQAQSLVYPFTGPIPNSLPQNILPLTQTPVVVPPFLQPEIMGVPKVKETMVPKHKEMPFPKYPVEPFTESQSLTLTDVEKLHLPLPLVQSWMHQPPQPLSPTVMFPPQSVLSLSQPKVLPVPQKAVPQRDMPIQAFLLYQEPVLGPVRGPFPILV

SEQ ID NO: 122 - protein, 207 aa, Ovis aries
REQEELNVVGETVESLSSSEESITHINKKIEKFQSEEQQQTEDELQDKIHPFAQAQSLVYPFTGPIPNSLPQNILPLTQTPVVVPPFLQPEIMGVPKVKETMVPKHKEMPFPKYPVEPFTESQSLTLTDVEKLHLPLPLVQSWMHQPPQPLPPTVMFPPQSVLSLSQPKVLPVPQKAVPQRDMPIQAFLLYQEPVLGPVRGPFPILV

SEQ ID NO: 123 - protein, 209 aa, Bubalus bubalis
RELEELNVPGEIVESLSSSEESITHINKKIEKFQSEEQQQMEDELQDKIHPFAQTQSLVYPFPGPIPKSLPQNIPPLTQTPVVVPPFLQPEIMGVSKVKEAMAPKHKEMPFPKYPVEPFTESQSLTLTDVENLHLPLPLLQSWMHQPPQPLPPTVMFPPQSVLSLSQSKVLPVPQKAVPYPQRDMPIQAFLLYQEPVLGPVRGPFPIIV

SEQ ID NO: 124 - protein, 217 aa, Camelus dromedarius
REKEEFKTAGEALESISSSEESITHINKQKIEKFKIEEQQQTEDEQQDKIYTFPQPQSLVYSHTEPIPYPILPQNFLPPLQPAVMVPFLQPKVMDVPKTKETIIPKRKEMPLLQSPVVPFTESQSLTLTDLENLHLPLPLLQSLMYQIPQPVPQTPMIPPQSLLSLSQFKVLPVPQQMVPYPQRAMPVQAVLPFQEPVPDPVRGLHPVPQPLVPVIA

SEQ ID NO: 125 - protein, 217 aa, Camelus bactrianus
REKEEFKTAGEALESISSSEESITHINKQKIEKFKIEEQQQTEDEQQDKIYTFPQPQSLVYSHTEPIPYPILPQNFLPPLQPAVMVPFLQPKVMDVPKTKETIIPKRKEMPLLQSPVVPFTESQSLTLTDLENLHLPLPLLQSLMYQIPQPVPQTPMIPPQSLLSLSQFKVLPVPQQMVPYPQRAIPVQAVLPFQEPVPDPVRGLHPVPQPLVPVIA

SEQ ID NO: 126 - protein, 209 aa, Bos mutus; identical to SEQ ID NO: 6

SEQ ID NO: 127 - protein, 226 aa, Equus caballus; identical to SEQ ID NO: 105

SEQ ID NO: 128 - protein, 226 aa, Equus asinus
REKEELNVSSETVESLSSNEPDSSSEESITHINKEKSQKFKHEGQQQREVEHQDKISRFVQPQPVVYPYAEPVPYAVVPQNILVLAQPPIVPFLQPEIMEVSQAKETILPKRKVMPFLKSPIVPFSERQILNPTNGENLRLPVHLIQPFMHQVPQSLLQTLMLPSQPVLSPPQSKVAPFPQPVVPYPQRDTPVQAFLLYQDPQLGLTGEFDPATQPIVPVHNPVIV

SEQ ID NO: 129 - protein, 141 aa, Alces alces
  VARIANT 6: note = X is any amino acid
  VARIANT 17: note = X is any amino acid
  VARIANT 65: note = X is any amino acid
IHPFAXTQSLVYPFTGXIPYSLPQNFLPLPQTPGMVPPFLQPEIMGVSEVKETMVPKNKEMPFPXYPVEPFAEGQSLTLTDVENLHLPLPLLQSWMHQTPQPLPPTVMFPPQSVLSLSQPKVLSVPQKAVPYPQRDMPIQA

SEQ ID NO: 130 - protein, 173 aa, Vicugna pacos
DEQQDKIYTFPQPQSLVYSHTEPIPYPILPQNFLPPLQPAVMVPFLQPKVMDVPKTKEIVIPKRKEMPLLQSPLVPFTESQSLTLTDLENLHLPLPLLQSLMHQIPQPVPQTPMIPPQSLLSLSQFKVLPVPQQMVPYPQRAMPVQALLPFQEPIPDPVRGLHPVPQPLVPVI

SEQ ID NO: 131 - protein, 209 aa, Bos indicus; identical to SEQ ID NO: 6

SEQ ID NO: 132 - protein, 217 aa, Lama glama
REKEEFKTAGEAVESISSSEESITHINKQKIEKFKIEEQQQTEDEQQDKIYTFPQPQSLVYSHTEPIPYPILPQNFLPPLQPAVMVPFLQPKVMDVPKTKEIVIPKRKEMPLLQSPLVPFTESQSLTLTDLENLHLPLPLLQSLMHQIPQPVPQTPMIPPQSLLSLSQFKVLPVPQQMVPYPQRAMPVQALLPFQEPIPDPVRGLHPVPQPLVPVIA

SEQ ID NO: 133 - protein, 211 aa, Homo sapiens
RETIESLSSSEESITEYKQKVEKVKHEDQQQGEDEHQDKIYPSFQPQPLIYPFVEPIPYGFLPQNILPLAQPAVVLPVPQPEIMEVPKAKDTVYTKGRVMPVLKSPTIPFFDPQIPKLTDLENLHLPLPLLQPLMQQVPQPIPQTLALPPQPLWSVPQPKVLPIPQQVVPYPQRAVPVQALLLNQELLLNPTHQIYPVTQPLAPVHNPISV

SEQ ID NO: 134-147 - skipped (14 entries)

SEQ ID NO: 148 - protein, 190 aa, Bos taurus
MMKSFFLVVTILALTLPFLGAQEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEDSPEVIESPPEINTVQVTSTAV

SEQ ID NO: 149 - protein, 190 aa, Bos taurus
MMKSFFLVVTILALTLPFLGAQEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTIEAVESTVATLEASPEVIESPPEINTVQVTSTAV

SEQ ID NO: 150 - protein, 190 aa, Bos taurus
MMKSFFLVVTILALTLPFLGAQEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTIEAVESTVATLEASPEVTESPPEINTVQVTSTAV

SEQ ID NO: 151 - protein, 169 aa, Bos taurus
QEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMAHHPHPHLSFMAIPPKKNQDKTEIPTINTIASGEPTSTPITEAVESTVATLEASPEVIESPPEINTVQVTSTAV

SEQ ID NO: 152 - protein, 190 aa, Bos taurus
MMKSFFLVVTILALTLPFLGAQEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTGIPTINTIASGEPTSTPTIEAVESTVATLEASPEVTESPPEINTVQVTSTAV

SEQ ID NO: 153 - protein, 160 aa, Bos taurus
RCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPITEAVESTVATLEDSPEVIESPPEINTVQVTSTAV

SEQ ID NO: 154 - protein, 160 aa, Bos taurus
HCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEDSPEVIESPPEINTVQVTSTAV

SEQ ID NO: 155 - protein, 179 aa, Bos taurus
MMKSFFLVVTILALTLPFLRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTIEAVESTVATLEASPEVIESPPEINTVQVTSTAV

SEQ ID NO: 156 - protein, 159 aa, Bos taurus
CEKDERFFSDKIAKYIPIQYVLSRYPSYGLNYYQQKPVALINNQFLPYPYYAKPAAVRSPAQILQWQVLSNTVPAKSCQAQPTTMAHHPHPHLSFMAIPPKKNQDKTEIPTINTIASGEPTSTPTTEAVESTVATLEDSPEVIESPPEINTVQVTSTAV

SEQ ID NO: 157 - protein, 160 aa, Bos taurus
RCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTIEAVESTVATLEASPEVIESPPEINTVQVTSTAV

SEQ ID NO: 158 - protein, 160 aa, Bos taurus
RCEKDERFFSDKIAKYIPIQYVLSRYPSYGLNYYQQKPVALINNQFLPYPYYAKPAAVRSPAQILQWQVLSNTVPAKSCQAQPTTMACHPHPHLSFMAIPPKKNQDKTEIPTINTIASGEPTSTPITEAVESTVATLEDSPEVIESPPEINTVQVTSTAV

SEQ ID NO: 159 - protein, 145 aa, Bos taurus
YIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEDSPEVIESPPEINTVQVTSTAV

SEQ ID NO: 160 - protein, 160 aa, Bos taurus
MMKSFFLVVTILALTLPFLGAQEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAV

SEQ ID NO: 161 - protein, 142 aa, Bos taurus
IQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPITEAVESTVATLEDSPEVIESPPEINTVQVTSTAV

SEQ ID NO: 162 - protein, 141 aa, Bos taurus
QYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEDSPEVIEGPPEINTVQVTSTAV

SEQ ID NO: 163 - protein, 139 aa, Bos taurus
VLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEDSPEVIESPPEINTVQVTSTAV

SEQ ID NO: 164 - protein, 140 aa, Bos taurus
YVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTIEAVESTVATLEASPEVIESPPEINTVQVTSTAV

SEQ ID NO: 165 - protein, 192 aa, Capra hircus
MMKSFFLVVTILALTLPFLGAQEQNQEQPICCEKDERFFDDKIAKYIPIQYVLSRYPSYGLNYYRQRPVALINNQFLPYPYYAKPIAVRSPAQTLQWQVLPNTVPAKSCQDQPTTLARHPHPHLSFMAIPPKKDQDKTEIPAINTIASAEPTVHSTPTTEAIVNTVDNPEASSESIASAPETNTAQVTSTEV

SEQ ID NO: 166 - protein, 192 aa, Capra hircus
MMKSFFLVVTILALTLPFLGAQEQNQEQPICCEKDERFFDDKIAKYIPIQYVLSRYPSYGLNYYQQRPVALINNQFLPYPYYAKPVAVRSPAQTLQWQVLPNTVPAKSCQDQPTTLARHPHPHLSFMAIPPKKDQDKTEIPAINTIASAEPTVHSTPTTEAIVNTVDNPEASSESIASASETNTAQVTSTEV

SEQ ID NO: 167 - protein, 162 aa, Capra hircus
CCEKDERFFDDKIAKYIPIQYVLSRYPSYGLNYYQQRPVALINNQFLPYPYYAKPVAVRSPAQTLQWQVLPNTVPAKSCQDQPTTLARHPHPHLSFMAIPPKKDQDKTEIPAINTIASAEPTVHSTPTTEAIVNTVDNPEASSESIASAPETNTAQVTSTEV

SEQ ID NO: 168 - protein, 161 aa, Capra hircus
CEKDERFFDDKIAKYIPIQYVLSRYPSYGLNYYQQRPVALINNQFLPYPYYAKPIAVRSPAQTLQWQVLPNTVPAKSCQDQPTTLARHPHPHLSFMAIPPKKDQDKTEIPAINTIASAEPTVHSTPTTEAIVNTVDNPEASSESIASAPETNTAQVTSTEV

SEQ ID NO: 169 - protein, 192 aa, Capra hircus
MMKSFFLVVTILALTLPFLGAQEQNQEQPICCEKDERFFDDKIAKYIPIQYVLSRYPSYGLNYYQQRPVALINNQFLPYPYYAKPVAVRSPAQTLQWQVLPNTVPAKSCQDQPTTLARHPHPHLSFMAIPPKKDQDKTEVPAINTIASAEPTVHSTPTTEAIVNTVDNPEASSESIASASETNTAQVTSTEV

SEQ ID NO: 170 - protein, 162 aa, Capra hircus
CCEKDERFFDDKIAKYIPIQYVLSRYPSYGLNYYQQRPVALINNQFLPYPYYAKPIAVRSPAQTLQWQVLPNTVPAKSCQDQPTTLARHPHPHLSFMAIPPKKDQDKTEIPAINTIASAEPTVHSTPTTEAIVNTVDNPEASSESIASAPETNTAQVTSTEV

SEQ ID NO: 171 - protein, 161 aa, Capra hircus
CEKDERFFDDKIAKYIPIQYVLSRYPSYGLNYYQQRPVALINNQFLPYPYYAKPIAVRSPAQTLQWQVLPNTVPAKSCQDQPTTLARHPHPHLSFMAIPPKKDQDKTEIPAINTIASAEPTVHSTPTTEAIVNTVDNPEASSESIVSAPETNTAQVTSTEV

SEQ ID NO: 172 - protein, 162 aa, Capra hircus
CCEKDERFFDDKIAKYIPIQYVLSRYPSYGLNYYQQRPVALINNQFLPYPYYAKPIAVRSPAQTLQWQVLPNTVPAKSCQDQPTTLARHPHPHLSFMAIPPKKDQDKTEIPAINTIASAEPTVHSTPTTEAIVNTVDNPEASSESIVSAPETNTAQVTSTEV

SEQ ID NO: 173 - protein, 161 aa, Capra hircus
CEKDERFFDDKIAKYIPIQYVLSRYPSYGLNYYRQRPVALINNQFLPYPYYAKPIAVRSPAQTLQWQVLPNTVPAKSCQDQPTTLARHPHPHLSFMAIPPKKDQDKTEIPAINTIASAEPTVHSTPTTEAIVNTVDNPEASSESIASAPETNTAQVTSTEV

SEQ ID NO: 174 - protein, 192 aa, Capra hircus
MMKSFFLVVTILALTLPFLGAQEQNQEQPICCEKDERFFDDKIAKYIPIQYVLSRYPSYGLNYYQQRPVALINNQFLPYPYYAKPVAVRSPAQTLQWQVLPNTVPAKSCQDQPTTLARHPHPHLSFMAIPPKKDQDKTEIPAINTIASAEPTVHSTPITEAIVNTVDNPEASSESIASASETNTAQVTSTEV

SEQ ID NO: 175 - protein, 162 aa, Capra hircus
CCEKDERFFDDKIAKYIPIQYVLSRYPSYGLNYYQQRPVALINNQFLPYPYYAKPVAVRSPAQTLQWQVLPNTVPAKSCQNQPTTLARHPHPHLSFMAIPPKKDQDKTEIPAINTIASAEPTVHSTPTTEAIVNTADNPEASSESIASAPETNTAQVTSTEV

SEQ ID NO: 176 - protein, 162 aa, Capra hircus
CCEKDERFFDDKIAKYIPIQYVLSRYPSYGLNYYRQRPVALINNQFLPYPYYAKPIAVRSPAQTLQWQVLPNTVPAKSCQDQPTTLARHPHPHLSFMAIPPKKDQDKTEIPAINTIASAEPTVHSTPTTEAIVNTVDNPEASSESIASAPETNTAQVTSTEV

SEQ ID NO: 177 - protein, 162 aa, Capra hircus
CCEKDERFFDDKIAKYIPIQYVLSRYPSYGLNYYQQRPVALINNQFLPYPYYAKPVAVRSPAQTLQWQVLPNTVPAKSCQGQPTTLARHPHPHLSFMAIPPKKDQDKTEIPAINTIASAEPTVHSTPTTEAIVNTVDNPEASSESIASASETNTAQVTSTEV

SEQ ID NO: 178 - protein, 161 aa, Capra hircus
CEKDERFFDDKIAKYIPIQYVLSRYPSYGLNYYQQRPVALINNQFLPYPYYAKPVAVRSPAQTLQWQVLPNTVPAKSCQDQPTTLARHPHPHLSFMAIPPKKDQDKTEIPAINTIASAEPTVHSTPTTEAIVNTVDNPEASSESIASASETNTAQVTSTEV

SEQ ID NO: 179 - protein, 162 aa, Capra hircus
CCEKDERFFDDKIAKYIPIQYVLSRYPSYGLNYYQQRPVALINNQFLPYPYYAKPVAVRSPAQTLQWQVLPNTVPAKSCQDQPTTLARHPHPHLSFMAIPPKKDQDKTEIPAINTIASAEPTVHSTPTTEAIVNTVDNPEASSESIASASETNTAQVTSTEV

SEQ ID NO: 180 - protein, 161 aa, Capra hircus
CEKDERFFDDKIAKYIPIQYVLSRYPSYGLNYYQQRPVALINNQFLPYPYYAKPVAVRSPAQTLQWQVLPNTVPAKSCQDQPTTLARHPHPHLSFMAIPPKKDQDKTEVPAINTIASAEPTVHSTPTTEAIVNTVDNPEASSESIASASETNTAQVTSTEV

SEQ ID NO: 181 - protein, 162 aa, Capra hircus
CCEKDERFFDDKIAKYIPIQYVLSRYPSYGLNYYQQRPVALINNQFLPYPYYAKPIAVRSPAQTLQWQVLPNTVPAKSCQDQPTTLARHPHPHLSFMAIPPKKDQDKTEIPAINTIASAEPTVHSTPTTEAIVNTVDNPEASSESIASASETNTAQVTSTEV

SEQ ID NO: 182 - protein, 162 aa, Capra hircus
CCEKDERFFDDKIAKYIPIQYVLSRYPSYGLNYYQQRPVALINNQFLPYPYYAKPVAVRSPAQTLQWQVLPNTVPAKSCQDQPTTLARHPHPHLSFMAIPPKKDQDKTEVPAINTIASAEPTVHSTPTTEAIVNTVDNPEASSESIASASETNTAQVTSTEV

SEQ ID NO: 183 - protein, 162 aa, Capra hircus
CCEKDERFFDDKIAKYIPIQYVLSRYPSYGLNYYRQRPVALINNQFLPYPYYAKPVAVRSPAQTLQWQVLPNTVPAKSCQDQPTTLARHPHPHLSFMAIPPKKDQDKTEIPAINTIASAEPTVHSTPTTEAIVNTVDNPEASSESIASASETNTAQVTSTEV

SEQ ID NO: 184 - protein, 192 aa, Ovis aries
MMKSFFLVVTILALTLPFLGAQEQNQEQRICCEKDERFFDDKIAKYIPIQYVLSRYPSYGLNYYQQRPVALINNQFLPYPYYAKPVAVRSPAQTLQWQVLPNAVPAKSCQDQPTAMARHPHPHLSFMAIPPKKDQDKTEIPAINTIASAEPTVHSTPTTEAVVNAVDNPEASSESIASAPETNTAQVTSTEV

SEQ ID NO: 185 - protein, 162 aa, Ovis aries
CCEKDERFFDDKIAKYIPIQYVLSRYPSYGLNYYQQRPVALINNQFLPYPYYAKPVAVRSPAQTLQWQVLPNAVPAKSCQDQPTAMARHPHPHLSFMAIPPKKDQDKTEIPAINTIASAEPTVHSTPTTEAVVNAVDNPEASSESIASAPETNTAQVTSTEV

SEQ ID NO: 186 - protein, 190 aa, Bubalus bubalis
MMKSFFLVVTILALTLPFLGAQEQNQEQPIRCEKEERFFNDKIAKYIPIQYVLSRYPSYGLNYYQQKPVALINNQFLPYPYYAKPAAVRSPAQILQWQVLPNTVPAKSCQAQPTTMTRHPHPHLSFMAIPPKKNQDKTEIPTINTIVSVEPTSTPTTEAIENTVATLEASSEVIESVPETNTAQVTSTVV

SEQ ID NO: 187 - protein, 190 aa, Bubalus bubalis
MMKSFFLVVTILALTLPFLGAQEQNQEQPIRCEKEERFFNDKIAKYIPIQYVLSRYPSYGLNYYQQKPVALINNQFLPYPYYAKPAAVRSPAQILQWQVLPNTVPAKSCQAQPTTMTRHPHPHLSFMAIPPKKNQDKTEIPTINTIVSIEPTSTPTTEAIENTVATLEASSEVIESVPETNTAQVTSTVV

SEQ ID NO: 188 - protein, 190 aa, Bubalus bubalis
MMKSFFLVVTILALTLPFLGAQEQNQEQPIRCEKEERFFNDKIAKYIPIQYVLSRYPSYGLNYYQQKPVALINNQFLPYPYYAKPAAVRSPAQILQWQVLPNTVPAKSCQAQPTTMTRHPHPHLSFMAIPPKKNQDKTEIPTINTIVSVEPTSTPTTEAIENTVATLEASSDVIESVPETNTAQVTSTVV

SEQ ID NO: 189 - protein, 190 aa, Bubalus bubalis
MMKSFFLVVTILALTLPFLGAQEQNQEQPIRCEKEERFFNDKIAKYIPIQYVLSRYPSYGLNYYQQKPVALINNQFLPYPYYAKPAAVRSPAQILQWQVLPNTVPAKSCQAQPTTMTRHPHPHLSFMAIPPKKNQDKTEIPTINTIVSIEPTSTPTTEAIENTVATLEASSDVIESVPETNTAQVTSTVV

SEQ ID NO: 190 - protein, 190 aa, Bubalus bubalis
MMKSFFLVVTILALTLPFLGAQEQNQEQPIRCEKEERFFNDKIAKYIPIQYVLSRYPSYGLNYYQQKPVALINNQFLPYPYYAKPAAVRSPAQILQWQVLPNTVPAKSCQAQPTTMTRHPHPHLSFMAIPPKKNQDKTEIPTINTIVSVEPTSTPITEAIENTVATLEASSEVIESVPETNTAQVTSTVV

SEQ ID NO: 191 - protein, 190 aa, Bubalus bubalis
MMKSFFLVVTILALTLPFLGAQEQNQEQLIRCEKEERFFNDKIAKYIPIQYVLSRYPSYGLNYYQQKPVALINNQFLPYPYYAKPAAVRSPAQILQWQVLPNTVPAKSCQAQPTTMTRHPHPHLSFMAIPPKKNQDKTEIPTINTIVSVEPTSTPTTEAIENTVATLEASSDVIESVPETNTAQVTSTVV

SEQ ID NO: 192 - protein, 194 aa, Bos mutus
MMKSFFLVVTILALTLPFLGAQEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEASPEASPEVIESPPEINTVQVTSTAV

SEQ ID NO: 193 - protein, 183 aa, Bos mutus
MMKSFFLVVTILALTLPFLRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEASPEASPEVIESPPEINTVQVTSTAV

SEQ ID NO: 194 - protein, 976 aa, Bos mutus
MTSGTAVQRADSSEEKRHRKRKKHHLIKSQILSYTEDRGFNEGQMKEDEQEKADSSEEVRHFHSLQKDKVNMKFFIFTCLLAVALAKNVKSLLKSKKHGTITRTSFKVQESHRENVDSQTISSICQNFIILDCRSSATRILCKRFKITVDDKHYQKALNEINQFYQKFPQYLQYLYQGPIVLNPWDQVKRNAVPITPTLVIKLIFIFFFHKESTEVFTKKTKLTEEEKNRLNFLKKISQRYQKFALPQYLKTVYQHQKAMKPWIQPKTKVIPYVDYSDNIGSPYPVNPSASISYPVTPSASIPCPVNPSASIPCPMPDRDFTPPVYLTKTNLPNDPAVPSNPLVFTALGAPLYSIAGLPISPFSGQLPPTGVFRLVPVSSRPIGPLYSPNEAAPSANTYIVEILVPPTTPPHTVPDVQPLTITEPDEAEPAADALAAPEPQSSISFDQLIPQHLMSASNSNELLLNLNNAQLRPLQLQGPFNPWIPPFPGILQQQQQNQVPGLSPFSLSTREWFAGLVPNQIFVPGQVSFAQGTQAGQLDPSQPQTPQQTQRGPKNVMPSVFFKMPQEQAQMLQYYPVYMFLPWEQPQQTVAQSPPQTREQLFEEQMPFYTEFGYIPQQVEPVMPVEQQQPVFDPFLGTAPEIAAMDRKAVIVITIKRQLAPFVRTILEPVTSISYNTIPEKDRTGKFEVSQDTEGEARSVSGSDELSSQFFFPPYQYPRSHYSRFLCPWWAYFYPPIPVPASVSATTPLNEKKKYQIHVNKVERPTEPTAKQELTVTRKGAMMKSFFLVVTILALTLPFLGAQEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEASPEASPEVIESPPEINTVQVTSTAV

SEQ ID NO: 195 - protein, 190 aa, Bos indicus
MMKSFFLVVTILALTLPFLGAQEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPITEAVESTVATLEDSPEVIESPPEINTVQVTSTAV

SEQ ID NO: 196 - protein, 160 aa, Bos indicus
RCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTIEAVESTVATLEDSPEVIESPPEINTVQVTSTAV

SEQ ID NO: 197 - protein, 160 aa, Bos indicus
RCEKDERFFSDKIAKYIPIQYVLSRYPSYGLNYYQQKPVALINNQFLPYPYYAKPAAVRSSAQILQWQVLSNTVPAKSCQAQPTTMARHPHPHLSFMAIPPKKNQDKTEIPTINTIASGEPTSTPTTEAVESTVATLEDSPEVIESPPEINTVQVTSTAV

SEQ ID NO: 198 - protein, 160 aa, Bos indicus
RCEKDERFFSDKIAKYIPIQYVLSRYPSYGLNYYQQKPVALINNQFLPYPYYAKPAAVRSSAQILQWQVLSNTVPAKSCQAQPTTMARHPHPHLSFMAIPPKKNQDKTEIPTINTIASGEPTSTPTIEAVESTVATLEASPEVIESPPEINTVQVTSTAV

SEQ ID NO: 199 - protein, 171 aa, Bos indicus
RCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPITEAVESTVATLEDSPEVIESPPEINTVQVRQLQSKNLRRHQRRQR

SEQ ID NO: 200 - protein, 171 aa, Bos indicus
RCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPITEAVESTVATLEDSPEVIESPPEINTVQVLNLQSKNLRRHQRRQR

SEQ ID NO: 201 - protein, 156 aa, Bos indicus
DERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTIEAVESTVATLEASPEVIESPPEINTVQVTSTAV

SEQ ID NO: 202 - protein, 144 aa, Bos indicus
IPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEDSPEVIESPPEINTVQVTSTAV

SEQ ID NO: 203 - protein, 144 aa, Bos indicus
IPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPITEAVESTVATLEDSPEVIESPPEINTVQVTSTAV

SEQ ID NO: 204 - protein, 141 aa, Bos indicus
QYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTIEAVESTVATLEASPEVIESPPEINTVQVTSTAV

SEQ ID NO: 205 - protein, 139 aa, Bos indicus
VLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPITEAVESTVATLEDSPEVIESPPEINTVQVTSTAV

SEQ ID NO: 206 - protein, 210 aa, Jeotgalicoccus coquinae
MERPTEPTAKQELTVTRKGAMMKSFFLVVTILALTLPFLGAQEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTIEAVESTVATLEASPEVIESPPEINTVQVTSTAV

SEQ ID NO: 207 - protein, 184 aa, Jeotgalicoccus coquinae
LVVTILALTLPFLGAQEQNQEQLIRCEKEERFFNDKIAKYIPIQYVLSRYPSYGLNYYQQKPVALINNQFLPYPYYAKPAAVRSPAQILQWQVLPNTVPAKSCQAQPTTMTRHPHPHLSFMAIPPKKNQDKTEIPTINTIVSVEPTSTPTTEAIENTVATLEASSEVIESVPETNTAQVTSTVV

SEQ ID NO: 208 - protein, 190 aa, Bison bison
MMKSFFLVVTILALTLPFLGAQEQNQEQPIRCEKDERFFSDKIAKYIPIQYVLSRYPSYGLNAYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEASPEVIESPPEINTVQVTSTVV

SEQ ID NO: 209 - protein, 179 aa, Bison bison
MMKSFFLVVTILALTLPFLRCEKDERFFSDKIAKYIPIQYVLSRYPSYGLNAYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEASPEVIESPPEINTVQVTSTVV

SEQ ID NO: 210 - protein, 190 aa, Bos grunniens
MMKSFFLVVTILALTLPFLGAQEQNQEQPIRCEKDERFFSDKIAKYIPIQYVLSRYLSYGLNYYQQKPVALINNQFLPYPYYAKPAAVRSPAQILQWQVLSNTVPAKSCQAQPTTMARHPHPHLSFMAIPPKKNQDKTEIPTINTIASGERTSTPTTEAVESTVATLEASPEVIESPPEINTVQVTSTAV

SEQ ID NO: 211 - protein, 194 aa, Bos grunniens
MMKSFFLVVTILALTLPFLGAQEQNQEQPIRCEKDERFFSDKIAKYIPIQYVLSRYPSYGLNYYQQKPVALINNQFLPYPYYAKPAAVRSPAQILQWQVLSSTVPAKSCQAQPTTMARHPHPHLSFMAIPPKKNQDKTEIPTINTIASGEPTSTPTTEAVESTVATLEASPEASPEVIESPPEINTVQVTSTAV

SEQ ID NO: 212 - protein, 160 aa, Bos grunniens
RCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEASPEVIESPPEINTVQVTSTAV

SEQ ID NO: 213 - protein, 160 aa, Bos grunniens
RCEKDERFFSDKIAKYIPIQYVLSRYLSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEASPEVIESPPEINTVQVTSTAV

SEQ ID NO: 214 - protein, 164 aa, Bos grunniens
RCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEASPEASPEVIESPPEINTVQVTSTAV

SEQ ID NO: 215 - protein, 160 aa, Bos grunniens
RCEKDERFFSDKIAKYIPIQYVLSRYLSYGLNYYQQKPVALINNQFLPYPYYAKPAAVRSPAQILQWQVLSNTVPAKSCQAQPTTMARHPHPHLSFMAIPPKKNQDKTEIPTINTIASGERTSTPTTEAVESTVATLEASPEVIESPPEINTVQVTSTAV

SEQ ID NO: 216 - protein, 160 aa, unidentified; identical to SEQ ID NO: 96
  REGION 1..160: note = Bos indicus x Bos taurus

SEQ ID NO: 217 - protein, 160 aa, unidentified
  REGION 1..160: note = Bos indicus x Bos taurus
RCEKDERFFSDKIAKYIPIQYVLSRYPSYGLNYYQQKPVALINNQFLPYPYYAKPAAVRSPAQILQWQVLSNTVPAKSCQAQPTTMARHPHPHLAFMAIPPKKNQDKTEIPTINTIASGEPTSTPTTEAVESTVATLEDSPEVIESPPEINTVQVTSTAV

SEQ ID NO: 218 - protein, 192 aa, Oreamnos americanus
MMKSFFLVVTILALTLPFLGAQEQNQEQPICCEKDERFFDDKIAKYIPIQYVLSRYPSYGLNYYQQRPVALINNQFLPYPYYAKPVAVRSPAQILQWQVLPNTAPAKSCQDQPTTMARHPHPHLSFMAIPPKKDQDKTEIPTINTIASAEPTVHSTPTTEAIVNTVDNPEASSESIVSAPETNTAQVTSTEV

SEQ ID NO: 219 - protein, 192 aa, Naemorhedus goral
MMKSFFLVVTILALTLPFLGAQEQNQEQPICCEKDERFFDDKIAKYIPIQYVLSRYPSYGLNYYQQRPVALINNQFLPYPYYAKPVAVRSPAQTLQWQVLPNTAPAKSCQDQPTTMARHPHPHLSFMAIPPKKDQDKTEIPTINTIASAEPTVHSTPTTEAIVNTVDNPEASSESIASAPETNTAQVTSTEV

SEQ ID NO: 220 - protein, 190 aa, Odocoileus virginianus
MMKSFSLVVTILALTLPFLVAQEQNQEQPTGCEKDERFFNDKIVKYIPIQYVLSRYPSYGLSYYQHRPVALINNQFLPYPYYAKPGAVRSPAQILQWQVLPNTVPAKSCQAQPTTLARHPHPRLSFMAIPPKKNQDKTDIPTINTIATVESTITPTTEAIVDTVATPEASSEVIESAPETKTDQVTSTVV

SEQ ID NO: 221 - protein, 192 aa, Capricornis sumatraensis
MMKSFFLVVTILALTLPFLGAQEQNQEQPICCEKDERFFDDKIAKYIPIQYVLSRYPSYGLNYYQQRPVALINNQFLPYPYYAKPVAVRSPAQTLQWQVLPNTAPAKSCQDQPTTMARHPHPHLSFMAIPPKKDQDKTEIPTINTIASAEPTVHSTPTTEAIVNTVDNPEASSESIVSAPETNTAQVTSTEV

SEQ ID NO: 222 - protein, 192 aa, Capricornis crispus
MMKSFFLAVTILALTLPFLGAQEQNQEQPICCEKDETFFDDKIAKYIPIQYVLSRYPSYGLNYYQQRPVALINNQFLPYPYYAKPVAVRSPAQTLQWQVLPNTVPAKSCQDQPTTMARHPHPHLSFMAIPPKKDQDKTEIPTINTIASAEPTVHSTPTTEAIVNTVDNPEASSESIVSAPETNTAQVTSTEV

SEQ ID NO: 223 - protein, 192 aa, Capricornis crispus
MMKSFFLVVTILALTLPFLGAQEQNQEQPICCEKDETFFDDKIAKYIPIQYVLSRYPSYGLNYYQQRPVALINNQFLPYPYYAKPVAVRSPAQTLQWQVLPNTVPAKSCQDQPTTMARHPHPHLSFMAIPPKKDQDKTEIPTINTIASAEPTVHSTPTTEAIVNTVDNPEASSESIVSAPETNTAQVTSTEV

SEQ ID NO: 224 - protein, 192 aa, Capricornis swinhoei [sequence omitted]

SEQ ID NO: 225 - protein, 202 aa, Saiga tatarica
MMKSFFLVVTILALTLPFLDAQERNQEQPICCEKDERFFNDRIAKYIPIQYVLSRYPSYGLNYYQQRPVALINNQFLPYPYYAKPVAVRSPAQTLQWQVLPNTVPAKSCQDQPTTMARHPHPHLSFMAIPPKKDQDKTEIPTINTVASAEPASTPTTEAIVNTEAIVNTEAIVNTVDNPEASSEIIASVPETNTAQVTSTEV

SEQ ID NO: 226 - protein, 192 aa, Rupicapra rupicapra
MMKSFFLVATILALTLPFLGAQEQNQEQSICCEKDERFFEDKIAKYIPIQYVLSRYPSYGLNYYQQRPVALINNQFLPYPYYAKPVAVRSPAQTLQWQVLPNTAPAKSCQDQPTTMAHHPHPHLSFMAIPPKKDQDKTEIPTINTIASAEPTVHSTPTTEAIVNTVDNPEASSESIASAPETNTAQVTSTEV

SEQ ID NO: 227 - protein, 190 aa, Cervus nippon
MMKSFFLAVTILALTLPFLVAQEQIQEQPTGCEKDERFFNDKIVKYIPIQYALSRYPSYGLNYYQHRPVALINNQFLPYPYYVKPGAVRSPAQILQWQVLPNTVPAKFCQAQPTTMARHPHPRLSFMAIPPKKNQDKTDIPSINTIATAESTITPTTEAIVDTVATQEAFSEVIESAPEAKTDQVTSTVV

SEQ ID NO: 228 - protein, 139 aa, Bos frontalis
VLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEASPEVIESPPEINTVQVTSTAV

SEQ ID NO: 229 - protein, 170 aa, Muntiacus reevesi
AQELNQEQPTGCEKDERFFNDKIVKYIPIQYALSRYPSYGLNYYQHRPVALISNQFLPYPYYAKPGAVRSPAQILQWQALPNTVPATSCQAQPATVARHPHPRLSFMAIPPKKSQDKTDHPTINTIATVESTATPTTEAVVDTAATQEASPEVIASAPEASTDQVTSTAV

SEQ ID NO: 230 - protein, 170 aa, Muntiacus muntjak
AQELNQEQPTGCEKDERFFNDKIVKYIPIQYALSRYPSYGLNYYQHRPVALISNQFLPYPYYAKPGAVRSPAQTLQWQALPNTVPATSCQAQPATMARHPHPRLSFMAIPPKKTQDKTDHPTFNTIATAESTATPTTEAVVDTVATQEASSEVTASAPEANTDQVTSTAV

SEQ ID NO: 231 - protein, 160 aa, Madoqua saltiana
CCEKDERFFNDKIAKYIPIQYVLSRYPSYGLNYYQQRPVALINNQFLPYPYFAKPVAVRSPAQTLQWQVLPNTVPAKSCQDQPTTMARHPHPHLSFMAIPPKKDQDKPEIPTINTIASAELTSTPTTEAIVNTVDNPEASSEIIASVPETNTAEVTSTEV

SEQ ID NO: 232 - protein, 172 aa, Gazella dorcas
CCEKDERFFNDKIAKYIPIQYVLSRYPSYGLNYYQQRPVALINNQFLPYPYYAKPVAVRSPAQTLQWQVLPNTVPAKSCQDQPTTMARHPHPHLSFMAIPPKKDQDKTEIPTINTIASAEPASTPTTEAIVNTEAIVNTEPIVNTVDNPEASSEIIASVPETNTAQVTSTEV

SEQ ID NO: 233 - protein, 178 aa, Gazella arabica
  VARIANT 178: note = X is any amino acid
CCEKDERFFNDKIAKYIPIQYVLSRYPSYGLNYYQQRPVALINNQFLPYPYYAKPVAVRSPAQTLQWQVLPNTVPAKSCQDQPTTMARHPHPHLSFMAIPPKKDQDKTEIPTINTIASAEPASTPTTEAIVNTEAVVNTEAIVNTEAIVNTVDNPEASSEIIASVPETNTAQVTSTEX

SEQ ID NO: 234 - protein, 162 aa, Capra ibex
CCEKDERFFDDKIAKYIPIQYVLNRYPTYGLNYYQQRPVALINNQFLPYPYYAKPVAVRSPAQTLQWQVLPNTVPAKSCQDQPTTLARHPHPHLSFMAIPPKKDQDKTEIPAINTIASAEPTVHSTPTTEAIVNTVDNPEASSESIASAPETNTAQVTSTEV

SEQ ID NO: 235 - protein, 162 aa, Ovis ammon
  VARIANT 73: note = X is any amino acid
CCEKDERFFDDKIAKYIPIQYVLSRYPSYGLNYYQQRPVALINNQFLPYPYYAKPVAVRSPAQTLQWQVLPNXVPAKSCQDQPTAMARHPHPHLSFMAIPPKKDQDKTEIPAINTIASAEPTVHSTPTTEAVVNAVDNPEASSESIASAPETNTAQVTSTEV

SEQ ID NO: 236 - protein, 162 aa, Ovis orientalis
CCEKDERFFDDKIAKYIPIQYVLSRYPSYGLNYYQQRPVALINNQFLPYPYYAKPVAVRSPAQTLQWQVLPNTVPAKSCQDQPTAMARHPHPHLSFMAIPPKKDQDKTEIPAINTIASAEPTVHSTPTTEAVVNAVDNPEASSESIASAPETNTAQVTSTEV

SEQ ID NO: 237 - protein, 162 aa, Ovis orientalis
CCEKDERFFDDKIAKYIPIQYVLSRYPSYGLNYYQQRPVALINNQFLPYPYYAKPVAVRSPAQTLQWQVLPNAVPAKSCQDQPTAMARHPHPHLSFMAIPPKKDQDKTEIPAINTIASAEPTVHSTPTTEAVVNAVDNPEASSESIASAPETNTAQITSTEV

SEQ ID NO: 238 - protein, 463 aa, Cervus hanglu
QEVKVTVDNKHYQKVLNEISQFYQKFPQYLQYLYQGPIVMNPWEQVKRTAGPFIPTVVSAAFLYVSCLFGFLFKTKLTEEEKNRLNFLKKISQYYQKFAWPQYLKTVYQYQKAMKPWTQPKTNAIPYVLIPQRLMSASNSNELLLNLNNAQLQPLQLQGPFNSWIPPFPGILQQQQQDQIPGLSPFSLSTLERFAGLVPNQIFVPGQVGFAQGTQAGQLDPSQPQTPQQTQRGPKNVMPSLLSLKMPQEQAQLLQYYPVYMFLPWEQPQQTVAQSPPQTRLQLFEEQMPYYTEFGYIPQQVEPGCEKDERFFNDKIVKYIPIQYALSRYPSYGLNYYQHRPVALINNQFLPYPYYVKPGAVRSPAQILQWQVLPNTVPAKFCQAQPTTMARHPHPRLSFMAIPPKKNQDKTDIPSINTIATAESTITPTTEAIVDTVATQEASSEVIESAPEAKTDQVTSTVV

SEQ ID NO: 239 - protein, 160 aa, Procapra gutturosa
CCEKDERFFNDKIAKYIPIQYVMSRYPSYGLNYYQQRPVALINNQFLPYPYYAKPVAVRSPAQTLQWQVLPNTVPAKSCQDQPTTMARHPHPHLSFMAIPPKKDQDKTEIPTINTIASAEPTSTPTTEAIVNTVDNPEATSEIIASVPETNTAQVTSTEV

SEQ ID NO: 240 - protein, 160 aa, Procapra gutturosa
  VARIANT 111: note = X is any amino acid
  VARIANT 121: note = X is any amino acid
  VARIANT 125: note = X is any amino acid
  VARIANT 139: note = X is any amino acid
CCEKDERFFNDKIAKYIPIQYVMSRYPSYGLNYYQQRPVALINNQFLPYPYYAKPVAVRSPAQTLQWQVLPNTVPAKSCQDQPTTMARHPHPHLSFMAIPPKKDQDKTEIXTINTIASAEXTSTXTTEAIVNTVDNPEXTSEIIASVPETNTAQVTSTEV

SEQ ID NO: 241 - protein, 190 aa, Bos taurus; identical to SEQ ID NO: 150

SEQ ID NO: 242 - protein, 190 aa, Bos taurus; identical to SEQ ID NO: 152

SEQ ID NO: 243 - protein, 190 aa, Bos taurus; identical to SEQ ID NO: 149

SEQ ID NO: 244 - protein, 190 aa, Bos taurus; identical to SEQ ID NO: 148

SEQ ID NO: 245 - protein, 160 aa, Bos taurus; identical to SEQ ID NO: 160

SEQ ID NO: 246 - protein, 169 aa, Bos taurus; identical to SEQ ID NO: 151

SEQ ID NO: 247 - protein, 179 aa, Bos taurus; identical to SEQ ID NO: 155

SEQ ID NO: 248 - protein, 160 aa, Bos taurus; identical to SEQ ID NO: 157

SEQ ID NO: 249 - protein, 160 aa, Bos taurus; identical to SEQ ID NO: 153

SEQ ID NO: 250 - protein, 106 aa, Bos taurus
AEQQNEEEPIRCEKDERFFSDKIAKYIPIQYVLSRYPSYGLNYYQQKPVALINNQFLPYPYYAKPAAVRSPAQILQWQVLSNTVPAKSCQAQPTTMARHPPHHLSF

SEQ ID NO: 251 - protein, 160 aa, Bos taurus; identical to SEQ ID NO: 154

SEQ ID NO: 252 - protein, 160 aa, Bos taurus; identical to SEQ ID NO: 158

SEQ ID NO: 253 - protein, 159 aa, Bos taurus; identical to SEQ ID NO: 156

SEQ ID NO: 254 - protein, 210 aa, Jeotgalicoccus coquinae; identical to SEQ ID NO: 206

SEQ ID NO: 255 - protein, 184 aa, Jeotgalicoccus coquinae; identical to SEQ ID NO: 207

SEQ ID NO: 256 - protein, 194 aa, Bos mutus; identical to SEQ ID NO: 192

SEQ ID NO: 257 - protein, 183 aa, Bos mutus; identical to SEQ ID NO: 193

SEQ ID NO: 258 - protein, 976 aa, Bos mutus; identical to SEQ ID NO: 194

SEQ ID NO: 259 - protein, 190 aa, Bos indicus; identical to SEQ ID NO: 195

SEQ ID NO: 260 - protein, 160 aa, Bos indicus; identical to SEQ ID NO: 196

SEQ ID NO: 261 - protein, 171 aa, Bos indicus; identical to SEQ ID NO: 199

SEQ ID NO: 262 - protein, 171 aa, Bos indicus; identical to SEQ ID NO: 200

SEQ ID NO: 263 - protein, 160 aa, Bos indicus; identical to SEQ ID NO: 197

SEQ ID NO: 264 - protein, 160 aa, Bos indicus; identical to SEQ ID NO: 198

SEQ ID NO: 265 - protein, 156 aa, Bos indicus; identical to SEQ ID NO: 201

SEQ ID NO: 266 - protein, 194 aa, Bos grunniens; identical to SEQ ID NO: 211

SEQ ID NO: 267 - protein, 190 aa, Bos grunniens; identical to SEQ ID NO: 210

SEQ ID NO: 268 - protein, 160 aa, Bos grunniens; identical to SEQ ID NO: 212

SEQ ID NO: 269 - protein, 164 aa, Bos grunniens; identical to SEQ ID NO: 214

SEQ ID NO: 270 - protein, 160 aa, Bos grunniens; identical to SEQ ID NO: 213

SEQ ID NO: 271 - protein, 190 aa, Bison bison; identical to SEQ ID NO: 208

SEQ ID NO: 272 - protein, 179 aa, Bison bison; identical to SEQ ID NO: 209

SEQ ID NO: 273 - protein, 190 aa, Bubalus bubalis; identical to SEQ ID NO: 186

SEQ ID NO: 274 - protein, 190 aa, Bubalus bubalis; identical to SEQ ID NO: 190

SEQ ID NO: 275 - protein, 190 aa, Bubalus bubalis; identical to SEQ ID NO: 187

SEQ ID NO: 276 - protein, 190 aa, Bubalus bubalis; identical to SEQ ID NO: 189

SEQ ID NO: 277 - protein, 190 aa, Bubalus bubalis; identical to SEQ ID NO: 188

SEQ ID NO: 278 - protein, 190 aa, Bubalus bubalis; identical to SEQ ID NO: 191

SEQ ID NO: 279 - protein, 192 aa, Oreamnos americanus; identical to SEQ ID NO: 218

SEQ ID NO: 280 - protein, 192 aa, Capricornis swinhoei [sequence omitted]

SEQ ID NO: 281 - protein, 192 aa, Naemorhedus goral; identical to SEQ ID NO: 219

SEQ ID NO: 282 - protein, 192 aa, Capricornis sumatraensis; identical to SEQ ID NO: 221

SEQ ID NO: 283 - protein, 192 aa, Capricornis crispus; identical to SEQ ID NO: 222

SEQ ID NO: 284 - protein, 192 aa, Capricornis crispus; identical to SEQ ID NO: 223

SEQ ID NO: 285 - protein, 202 aa, Saiga tatarica; identical to SEQ ID NO: 225

SEQ ID NO: 286 - protein, 160 aa, unidentified; identical to SEQ ID NO: 96
  REGION 1..160: note = Bos indicus x Bos taurus

SEQ ID NO: 287 - protein, 160 aa, unidentified; identical to SEQ ID NO: 217
  REGION 1..160: note = Bos indicus x Bos taurus

SEQ ID NO: 288 - protein, 192 aa, Capra hircus; identical to SEQ ID NO: 169

SEQ ID NO: 289 - protein, 192 aa, Capra hircus; identical to SEQ ID NO: 166

SEQ ID NO: 290 - protein, 192 aa, Capra hircus; identical to SEQ ID NO: 174

SEQ ID NO: 291 - protein, 192 aa, Capra hircus; identical to SEQ ID NO: 165

SEQ ID NO: 292 - protein, 161 aa, Capra hircus; identical to SEQ ID NO: 168

SEQ ID NO: 293 - protein, 161 aa, Capra hircus; identical to SEQ ID NO: 171

SEQ ID NO: 294 - protein, 162 aa, Capra hircus; identical to SEQ ID NO: 167

SEQ ID NO: 295 - protein, 162 aa, Capra hircus; identical to SEQ ID NO: 177

SEQ ID NO: 296 - protein, 162 aa, Capra hircus; identical to SEQ ID NO: 175

SEQ ID NO: 297 - protein, 161 aa, Capra hircus; identical to SEQ ID NO: 180

SEQ ID NO: 298 - protein, 162 aa, Capra hircus; identical to SEQ ID NO: 170

SEQ ID NO: 299 - protein, 162 aa, Capra hircus; identical to SEQ ID NO: 172

SEQ ID NO: 300 - protein, 161 aa, Capra hircus; identical to SEQ ID NO: 178

SEQ ID NO: 301 - protein, 162 aa, Capra hircus; identical to SEQ ID NO: 182

SEQ ID NO: 302 - protein, 162 aa, Capra hircus; identical to SEQ ID NO: 179

SEQ ID NO: 303 - protein, 159 aa, Capra hircus
CCEKDERFFDDKIAKYIPIQYVLSRYPSYGLNYYQQRPVALINNQFLPYPYYAKPVAVRSPAQTLQWQVLPNTVPAKSCQDQPTTLARHPHPHLSFMAIPPKKDQDKTEIPAINTIASAEPTVHSTPTTEAIVNTVDNPEASSESIASASETNTAQVTS

SEQ ID NO: 304 - protein, 159 aa, Capra hircus
CCEKDERFFDDKIAKYIPIQYVLSRYPSYGLNYYQQRPVALINNQFLPYPYYAKPVAVRSPAQTLQWQVLPNTVPAKSCQDQPTTLARHPHPHLSFMAIPPKKDQDKTEVPAINTIASAEPTVHSTPTTEAIVNTVDNPEASSESIASASETNTAQVTS

SEQ ID NO: 305 - protein, 141 aa, Capra hircus
  VARIANT 110: note = X is any amino acid
CCEKDERFFDDKIAKYIPIQYVLSRYPSYGLNYYQQRPVALINNQFLPYPYYAKPVAVRSPAQTLQWQVLPNTVPAKSCQDQPTTLARHPHPHLSFMAIPPKKDQDKTEXPAINTIASAEPTVHSTPTTEAIVNTVDNPEA

SEQ ID NO: 306 - protein, 162 aa, Capra hircus; identical to SEQ ID NO: 181

SEQ ID NO: 307 - protein, 161 aa, Capra hircus; identical to SEQ ID NO: 173

SEQ ID NO: 308 - protein, 162 aa, Capra hircus; identical to SEQ ID NO: 176

SEQ ID NO: 309 - protein, 162 aa, Capra hircus; identical to SEQ ID NO: 183

SEQ ID NO: 310 - protein, 162 aa, Capra hircus
CCEKDERFFDDKIAKYIPIQYVLSRYPSYGLNYYQQRPVALINSQFLPYPYYAKPVAVRSPAQTLQWQVLPNTVPAKSCQDQPTTLARHPHPHLSFMAIPPKKDQDKTEIPAINTIASAEPTVHSTPTTEAIVNTVDNPEASSESIASASETNTAQVTSTEV

SEQ ID NO: 311 - protein, 190 aa, Odocoileus virginianus; identical to SEQ ID NO: 220

SEQ ID NO: 312 - protein, 192 aa, Rupicapra rupicapra; identical to SEQ ID NO: 226

SEQ ID NO: 313 - protein, 192 aa, Ovis aries; identical to SEQ ID NO: 184

SEQ ID NO: 314 - protein, 162 aa, Ovis aries; identical to SEQ ID NO: 185

SEQ ID NO: 315 - protein, 190 aa, Cervus nippon; identical to SEQ ID NO: 227

SEQ ID NO: 316 - protein, 178 aa, Gazella arabica; identical to SEQ ID NO: 233
  VARIANT 178: note = X is any amino acid

SEQ ID NO: 317 - protein, 170 aa, Muntiacus muntjak; identical to SEQ ID NO: 230

SEQ ID NO: 318 - protein, 170 aa, Muntiacus reevesi; identical to SEQ ID NO: 229

SEQ ID NO: 319 - protein, 172 aa, Gazella dorcas
  VARIANT 136: note = X is any amino acid
  VARIANT 151: note = X is any amino acid
  VARIANT 154: note = X is any amino acid
  VARIANT 161: note = X is any amino acid
  VARIANT 168: note = X is any amino acid
CCEKDERFFNDKIAKYIPIQYVLSRYPSYGLNYYQQRPVALINNQFLPYPYYAKPVAVRSPAQTLQWQVLPNTVPAKSCQDQPTTMARHPHPHLSFMAIPPKKDQDKTEIPTINTIASAEPASTPTTEAIVNTEPXVNTEPIVNTVDNPEXSSXIIASVPXTNTAQVXSTEV

SEQ ID NO: 320 - protein, 160 aa, Procapra gutturosa; identical to SEQ ID NO: 239

SEQ ID NO: 321 - protein, 160 aa, Procapra gutturosa; identical to SEQ ID NO: 240
  VARIANT 111: note = X is any amino acid
  VARIANT 121: note = X is any amino acid
  VARIANT 125: note = X is any amino acid
  VARIANT 139: note = X is any amino acid

SEQ ID NO: 322 - protein, 160 aa, Madoqua saltiana; identical to SEQ ID NO: 231

SEQ ID NO: 323 - protein, 155 aa, Ammotragus lervia
  VARIANT 138: note = X is any amino acid
CCEKDERFFDDKIAKYIPIQYVLSRYPSYGLNYYQQRPVALINNQFLPYPYYAKPVAVRSPAQTLQWQVLPNTAPAKSCQDQPTTMARHPHPHLSFMAIPPKKDQDKTEIPAINTIASVEPTVHSTPTAEAIVNTVDXPEASSESIASAPETNTA

SEQ ID NO: 324 - protein, 155 aa, Ammotragus lervia
CCEKDERFFDDKIAKYIPIQYVLSRYPSYGLNYYQQRPVALINNQFLPYPYYAKPVAVRSPAQTLQWQVLPNTAPAKSCQDQPTTMARHPHPHLSFMAIPPKKDQDKTEIPAINTIASVEPTVHSTPTAEAIVNTVDSPEASSESIASAPETNTA

SEQ ID NO: 325 - protein, 155 aa, Ammotragus lervia
CCEKDERFFDDKIAKYIPIQYVLSRYPSYGLNYYQQRPVALINNQFLPYPYYAKPVAVRSPAQTLQWQVLPNTAPAKSCQDQPTTMARHPHPHLSFMAIPPKKDQDKTEIPAINTIASVEPTVHSTPTAEAIVNTVDNPEASSESIASAPETNTA

SEQ ID NO: 326 - protein, 162 aa, Capra sibirica
CCEKDERFFDDKIAKYIPIQYVLSRYPSYGLNYYQQRPVALINNQFLPYPYYAKPVAVRSPAQTLQWQVLPNTVPAKSCQDQPTTLARHPHPHLSFMAIPPKKDQDKTEVPAINTIASAEPTVHSTPTTEAIVNTVDNPEASSESIASASETNTGQVTSTEV

SEQ ID NO: 327 - protein, 162 aa, Ovis canadensis
CCEKDERFFDDKIAKYIPIQYVLSRYPSYGLNYYQQRPVALINNQFLPYPYYAKPVAVRSPAQTLQWQVLPNTVPAKSCQDQPTTMAHHPHPHLSFMAIPPKKDQDKTEIPAINTIASVEPTVHSTPTTEAVVNTVDNPEASSESIASAPETNAAQVTTTEV

SEQ ID NO: 328 - protein, 162 aa, Ovis canadensis
  VARIANT 155..156: note = X is any amino acid
CCEKDERFFDDKIAKYIPIQYVLSRYPSYGLNYYQQRPVALINNQFLPYPYYAKPVAVRSPAQTLQWQVLPNTVPAKSCQDQPTTMAHHPHPHLSFMAIPPKKDQDKTEIPAINTIASVEPTVHSTPTTEAVVNTVDNPEASSESIASAPETNAXXVTTTEV

SEQ ID NO: 329 - protein, 178 aa, Gazella subgutturosa
CCEKNERFFNDKIAKYIPIQYVLSRYPSYGLNYYQQRPVALINNQFLPYPYYVKPVAVRSPAQTLQWQVLPNTVPAKSCQDQPTTMARHPHPHLSFMAIPPKKDQDKTEIPTINTIASAEPASTPTTEAIVNTEAVVNTEAIVNTEAIVNTVDNPEASSEIIASVPETNTAQVTSTEV

SEQ ID NO: 330 - protein, 172 aa, Antilope cervicapra
  VARIANT 33: note = X is any amino acid
  VARIANT 135: note = X is any amino acid
GCEKDERFFNDKIAKYIPIQYVLSRYPSYGLNXYQQRPVALINNQFLPYPYYVKPVAVRSPAQTLQWQVLPNTVPAKSCQDQPTTMARHPHPHLSFMAIPPKKDQDKTEIPTINTIASAEPASTPTTEAIVNTEXIVNTEPIVNTVDNPEASSEIIASVPETNTAQVTSTEV

SEQ ID NO: 331 - protein, 162 aa, Capra ibex; identical to SEQ ID NO: 234

SEQ ID NO: 332 - protein, 162 aa, Ovis vignei
CCEKDERFFDDKIAKYIPIQYVLSRYPSYGLNYYQQRPVALINNQFLPYPYYAKPVAVRSPAQTLQWQVLPNAVPAKSCQDQPTAMARHPHPHLSFMAIPPKKDQDKTEIPAINTIASAEPTVHSTPTTEAVVNAVDNPEASSESIASAPETNTAQVTSTES

SEQ ID NO: 333 - protein, 162 aa, Ovis vignei
  VARIANT 73: note = X is any amino acid
  VARIANT 162: note = X is any amino acid
CCEKDERFFDDKIAKYIPIQYVLSRYPSYGLNYYQQRPVALINNQFLPYPYYAKPVAVRSPAQTLQWQVLPNXVPAKSCQDQPTAMARHPHPHLSFMAIPPKKDQDKTEIPAINTIASAEPTVHSTPTTEAVVNAVDNPEASSESIASAPETNTAQVTSTEX

SEQ ID NO: 334 - protein, 162 aa, Ovis ammon
  VARIANT 156: note = X is any amino acid
CCEKDERFFDDKIAKYIPIQYVLSRYPSYGLNYYQQRPVALINNQFLPYPYYAKPVAVRSPAQTLQWQVLPNAVPAKSCQDQPTAMARHPHPHLSFMAIPPKKDQDKTEIPAINTIASAEPTVHSTPTTEAVVNAVDNPEASSESIASAPETNTAXVTSTEV

SEQ ID NO: 335 - protein, 162 aa, Ovis vignei
  VARIANT 112: note = X is any amino acid
CCEKDERFFDDKIAKYIPIQYVLSRYPSYGLNYYQQRPVALINNQFLPYPYYAKPVAVRSPAQTLQWQVLPNAVPAKSCQDQPTAMARHPHPHLSFMAIPPKKDQDKTEIPXINTIASAEPTVHSTPTTEAVVNAVDNPEASSESIASAPETNTAQVTSTEV

SEQ ID NO: 336 - protein, 162 aa, Ovis orientalis; identical to SEQ ID NO: 236

SEQ ID NO: 337 - protein, 162 aa, Ovis orientalis; identical to SEQ ID NO: 237

SEQ ID NO: 338 - protein, 162 aa, unidentified
  REGION 1..162: note = Ovis orientalis x vignei
  VARIANT 107: note = X is any amino acid
CCEKDERFFDDKIAKYIPIQYVLSRYPSYGLNYYQQRPVALINNQFLPYPYYAKPVAVRSPAQTLQWQVLPNAVPAKSCQDQPTAMARHPHPHLSFMAIPPKKDQDXTEIPAINTIASAEPTVHSTPTTEAVVNAVDNPEASSESIASAPETNTAQVTSTEV

SEQ ID NO: 339 - protein, 162 aa, Ovis vignei
  VARIANT 154: note = X is any amino acid
CCEKDERFFDDKIAKYIPIQYVLSRYPSYGLNYYQQRPVALINNQFLPYPYYAKPVAVRSPAQTLQWQVLPNAVPAKSCQDQPTAMARHPHPHLSFMAIPPKKDQDKTEIPAINTIASAEPTVHSTPTTEAVVNAVDNPEASSESIASAPETNXAQVTSTEV

SEQ ID NO: 340 - protein, 162 aa, Ovis ammon; identical to SEQ ID NO: 235
  VARIANT 73: note = X is any amino acid

SEQ ID NO: 341 - protein, 205 aa, Bos taurus
VSAALARPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 342 - protein, 215 aa, Bos taurus
MKLLILTCLVAVALARPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLWW

SEQ ID NO: 343 - protein, 206 aa, Bos taurus
LVAVALARPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 344 - protein, 214 aa, Bos taurus
MKLLILTCLVAVALARPKHPIKHQGLPQEVLNENLLRFFVAPFPEVFGKEKVNELSKDIGSESTEDQAMEDIKQMEAESISSSEEIVPNSVEQKHIQKDDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSGKTTMPLW

SEQ ID NO: 345 - protein, 213 aa, Bos taurus
MKLLILTCLVAVALARPKHPIKHQGLPQEVLNENLLRFFVAPFPEVFGKEKVNELSKDIGSESTEDQAMEDIKQMEAESISSSEEIVPNSVEKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 346 - protein, 214 aa, Bos taurus
MKLLILTCLVAVALARPKHPIKHQGLPQEVLNENLLRFFVAPFPEVFGKEKVNELSKDIGSESTEDQAMEDIKQMEAESISSSEEIVPNSVEKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLWW

SEQ ID NO: 347 - protein, 214 aa, Bos taurus
MKLLILTCLVAVALARPKHPIKHQGLPQEVLNENLLRFFVAPFPEVFGKEKVNELSKDIGSESTEDQAMEDIKEMEAESISSSGEIVPNSVEQKHIQKEDVPSERYLGYLEQLLRLKKYKVPQLEIVPNSAEERLHSMKEGIDAQQKEPMIGVNQELAYFYPELFRQFYQLDAYPSGAWYYVPLGTQYTDAPSFSDIPNPIGSENSEKTTMPLW

SEQ ID NO: 348 - protein, 195 aa, Bos taurus
PIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 349 - protein, 214 aa, Bos taurus
MKLLILTCLVAVALARPKHPIKHQGLPQEVLNENLLRFFVALFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIDAQQKEPMIGVNQELAYFYPELFRQFYQLDAYPSGAWYYVPLGTQYTDAPSFSDIPNPIGSENSEKTTISLW

SEQ ID NO: 350 - protein, 206 aa, Bos taurus
MKLLILTCLVAVALARPKHPIKHQGLPQEVLNENLLRFFVAPFPEVFGKEKVNELSKDIGSESTEDQAMEDIKQMEAESISSSEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 351 - protein, 206 aa, Bos taurus
MKLLILTCLVAVALARPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLERLHSMKEGIHAQQKEPMIGVNQELAYFYPELFRQFYQLDAYPSGAWYYVPLGTQYTDAPSFSDIPNPIGSENSEKTTMPLW

SEQ ID NO: 352 - protein, 207 aa, Bos taurus
MKLLILTCLVAVALARPKHPIKHQGLPQEVLNENLLRFFVAPFPEVFGKEKVNELSKDIGSESTEDQAMEDIKQMEAESISSSEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLWW

SEQ ID NO: 353 - protein, 206 aa, Bos taurus
MKLLILTCLVAVALARPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQLFRQFYQLDAYPSGAWYYVPLGTQYTDAPSFSDIPNPIGSENSEKTTMPLW

SEQ ID NO: 354 - protein, 205 aa, Bos taurus
MKLLILTCLVAVALARPKHPIKHQGLPQEVLNENLLRFFVAPFPEVFGKEKVNELSKDIGSESTEDQAMEDIKQMEAESISSSEEIVPNSVEKHIQKEDVPSERYLGYLEQLLRLKKYKVPQLERLHSMKEGIHAQQKEPMIGVNQELAYFYPELFRQFYQLDAYPSGAWYYVPLGTQYTDAPSFSDIPNPIGSENSEKTTMPLW

SEQ ID NO: 355 - protein, 201 aa, Bos taurus
MKLLILTCLVAVALARPKHPIKHQGLPQPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 356 - protein, 202 aa, Bos taurus
MKLLILTCLVAVALARPKHPIKHQGLPQPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLWW

SEQ ID NO: 357 - protein, 200 aa, Bos taurus
MKLLILTCLVAVALARPKHPIKHQGLPQEVLNENLLRFFVAPFPEVFGKEKVNELSKDIGSESTEDQAMEDIKQMEAESISSSEEIVPNSVEQKHIQKEDVPSERYLGYLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 358 - protein, 201 aa, Bos taurus
MKLLILTCLVAVALARPKHPIKHQGLPQEVLNENLLRFFVAPFPEVFGKEKVNELSKDIGSESTEDQAMEDIKQMEAESISSSEEIVPNSVEQKHIQKEDVPSERYLGYLEIVPNSAEERLHSMKEGIHAQQKEPMIGVNQELAYFYPELFRQFYQLDAYPSGAWYYVPLGTQYTDAPSFSDIPNPIGSENSEKTTMPLWW

SEQ ID NO: 359 - protein, 200 aa, Bos taurus
MKLLILTCLVAVALARPKHPIKHQGLPQPFPEVFGKEKVNELSKDIGSESTEDQAMEDIKQMEAESISSSEEIVPNSVEKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 360 - protein, 199 aa, Bos taurus
MKLLILTCLVAVALARPKHPIKHQGLPQEVLNENLLRFFVAPFPEVFGKEKVNELSKDIGSESTEDQAMEDIKQMEAESISSSEEIVPNSVEKHIQKEDVPSERYLGYLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 361 - protein, 200 aa, Bos taurus
MKLLILTCLVAVALARPKHPIKHQGLPQEVLNENLLRFFVAPFPEVFGKEKVNELSKDIGSESTEDQAMEDIKQMEAESISSSEEIVPNSVEKHIQKEDVPSERYLGYLEIVPNSAEERLHSMKEGIHAQQKEPMIGVNQELAYFYPELFRQFYQLDAYPSGAWYYVPLGTQYTDAPSFSDIPNPIGSENSEKTTMPLWW

SEQ ID NO: 362 - protein, 198 aa, Bos taurus
MKLLILTCLVAVALARPKHPIKHQGLPQEVLNENLLRFFVAPFPEVFGKEKVNELSKDIGSESTEDQAMEDIKQMEAESISSSEQKHIQKEDVPSERYLGYLEQLLRLKKYKVPQLERLHSMKEGIHAQQKEPMIGVNQELAYFYPELFRQFYQLDAYPSGAWYYVPLGTQYTDAPSFSDIPNPIGSENSEKTTMPLW

SEQ ID NO: 363 - protein, 198 aa, Bos taurus
MKLLILTCLVAVALARPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLERLHSMKEGIHAQQKEPMIGVNQLFRQFYQLDAYPSGAWYYVPLGTQYTDAPSFSDIPNPIGSENSEKTTMPLW

SEQ ID NO: 364 - protein, 214 aa, Bos taurus
  VARIANT 111..124: note = X is any amino acid
MKLLILTCLVAVALARPKHPIKHQGLPQEVLNENLLRFFVAPFPEVFGKEKVNELSKDIGSESTEDQAMEDIKQMEAESISSSEEIVPNSVEQKHIQKEDVPSERYLGYLXXXXXXXXXXXXXXEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 365 - protein, 193 aa, Bos taurus
MKLLILTCLVAVALARPKHPIKHQGLPQPFPEVFGKEKVNELSKDIGSESTEDQAMEDIKQMEAESISSSEEIVPNSVEQKHIQKEDVPSERYLGYLEQLLRLKKYKVPQLERLHSMKEGIHAQQKEPMIGVNQELAYFYPELFRQFYQLDAYPSGAWYYVPLGTQYTDAPSFSDIPNPIGSENSEKTTMPLW

SEQ ID NO: 366 - protein, 194 aa, Bos taurus
MKLLILTCLVAVALARPKHPIKHQGLPQPFPEVFGKEKVNELSKDIGSESTEDQAMEDIKQMEAESISSSEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLWW

SEQ ID NO: 367 - protein, 192 aa, Bos taurus
MKLLILTCLVAVALARPKHPIKHQGLPQEVLNENLLRFFVAPFPEVFGKEKVNELSKDIGSESTEDQAMEDIKQMEAESISSSEQKHIQKEDVPSERYLGYLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 368 - protein, 193 aa, Bos taurus
MKLLILTCLVAVALARPKHPIKHQGLPQEVLNENLLRFFVAPFPEVFGKEKVNELSKDIGSESTEDQAMEDIKQMEAESISSSEQKHIQKEDVPSERYLGYLEIVPNSAEERLHSMKEGIHAQQKEPMIGVNQELAYFYPELFRQFYQLDAYPSGAWYYVPLGTQYTDAPSFSDIPNPIGSENSEKTTMPLWW

SEQ ID NO: 369 - protein, 192 aa, Bos taurus
MKLLILTCLVAVALARPKHPIKHQGLPQEVLNENLLRFFVAPFPEVFGKEKVNELSKDIGSESTEDQAMEDIKQMEAESISSSEEIVPNSVEQKHIQKEDVPSERYLGYLEIVPNSAEERLHSMKEGIHAQQKEPMIGVNQLFRQFYQLDAYPSGAWYYVPLGTQYTDAPSFSDIPNPIGSENSEKTTMPLW

SEQ ID NO: 370 - protein, 172 aa, Bos taurus
FSEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 371 - protein, 187 aa, Bos taurus
MKLLILTCLVAVALARPKHPIKHQGLPQPFPEVFGKEKVNELSKDIGSESTEDQAMEDIKQMEAESISSSEEIVPNSVEQKHIQKEDVPSERYLGYLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 372 - protein, 166 aa, Bos taurus
  VARIANT 118: note = X is any amino acid
KEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFXQFYQLDAYPSGAWYYVPLGTQYTDAPSFSDIPNPIGSENSEKTTMPLW

SEQ ID NO: 373 - protein, 177 aa, Bos taurus
MKLLILTCLVAVALARPKHPIKHQGLPQEVLNENLLRFFVAPFPEVFGKEKVNELSKDIGSESTEDQAMEDIKEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 374 - protein, 178 aa, Bos taurus
MKLLILTCLVAVALARPKHPIKHQGLPQEVLNENLLRFFVAPFPEVFGKEKVNELSKDIGSESTEDQAMEDIKEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLWW

SEQ ID NO: 375 - protein, 155 aa, Bos taurus
GSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 376 - protein, 163 aa, Bos taurus
MKLLILTCLVAVALARPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPE

SEQ ID NO: 377 - protein, 177 aa, Bos taurus
MKLLILTCLVAVALARPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPEKSQVNSEGLHSYGL

SEQ ID NO: 378 - protein, 141 aa, Bos taurus
QMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 379 - protein, 136 aa, Bos taurus
SISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 380 - protein, 138 aa, Bos taurus
  VARIANT 10: note = X is any amino acid
  VARIANT 90: note = X is any amino acid
  VARIANT 128: note = X is any amino acid
AESISSSEEXVPNSVEQKHIQKEDVPSERYLGYLEQLLRLKKYKVPQLEIVPNSAEERLHSMKEGIHAQQKEPMIGVNQELAYFYPELFXQFYQLDAYPSGAWYYVPLGTQYTDAPSFSDIPNPIGSXNSEKTTMPLW

SEQ ID NO: 381 - protein, 126 aa, Bos taurus
NSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 382 - protein, 129 aa, Bos taurus
  VARIANT 81: note = X is any amino acid
IVPNSVEQKHIQKEDVPSERYLGYLEQLLRLKKYKVPQLEIVPNSAEERLHSMKEGIHAQQKEPMIGVNQELAYFYPELFXQFYQPDAYPSGAWYYVPLGTQYTDAPSFSDIPNPIGSENSEKTTMPLW

SEQ ID NO: 383 - protein, 111 aa, Bos taurus
ERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 384 - protein, 103 aa, Bos taurus
QLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 385 - protein, 214 aa, Capra hircus
MKLLILTCLVAVALARPKHPINHQGLSPEVLNENLLRFVVAPFPEVFRKENINELSKDIGSESTEDQAMEDAKQMKAGSSSSSEEIVPNSAQQKYIQKEDVPSERYLGYLEQLLRLKKYNVPQLEIVPKSAEEQLHSMKEGNPAHQKQPMIAVNQELAYFYPQLFRQFYQLDAYPSGAWYYLPLGTQYTDAPSFSDIPNPIGSENSGKTTMPLW

SEQ ID NO: 386 - protein, 214 aa, Capra hircus
MKLLILTCLVAVALARPKHPINHQGLSPEVLNENLLRFVVAPFPEVFRKENINELSKDIGSESTEDQAMEDAKQMKAGSSSSSEEIVPNSAEQKYIQKEDVPSERYLGYLEQLLKLKKYNVPQLEIVPKSAEEQLHSMKEGNPAHQKQPMIAVNQELAYFYPQLFRQFYQLDAYPSGAWYYLPLGTQYTDAPSFSDIPNPIGSENSGKTAMPLW

SEQ ID NO: 387 - protein, 213 aa, Capra hircus
KLLILTCLVAVALARPKHPINHQGLSPEVPNENLLRFVVAPFPEVFRKENINELSKDIGSESTEDQAMEDAKQMKAGSSSSSEEIVPNSAEQKYIQKEDVPSERYLGYLEQLLRLKKYNVPQLEIVPKSAEEQLHSMKEGNPAHQKQPMIAVNQELAYFYPQLFRQFYQLDAYPSGAWYYLPLGTQYTDAPSFSDIPNPIGSENSGKTTMPLW

SEQ ID NO: 388 - protein, 214 aa, Capra hircus
MKLLILTCLVAVALARPKHPINHRGLSPEVPNENLLRFVVAPFPEVFRKENINELSKDIGSESTEDQAMEDAKQMKAGSSSSSEEIVPNSAEQKYIQKEDVPSERYLGYLEQLLRLKKYNVPQLEIVPKSAEEQLHSMKEGNPAHQKQPMIAVNQELAYFYPQLFRQFYQLDAYPSGAWYYLPLGTQYTDAPSFSDIPNPIGSENSGKTTMPLW

SEQ ID NO: 389 - protein, 213 aa, Capra hircus
MKLLILTCLVAVALARPKHPINHQGLSPEVLNENLLRFVVAPFPEVFRKENINELSKDIGSESTEDQAMEDAKQMKAGSSSSSEEIVPNSAQKYIQKEDVPSERYLGYLEQLLRLKKYNVPQLEIVPKSAEEQLHSMKEGNPAHQKQPMIAVNQELAYFYPQLFRQFYQLDAYPSGAWYYLPLGTQYTDAPSFSDIPNPIGSENSGKTTMPLW

SEQ ID NO: 390 - protein, 213 aa, Capra hircus
MKLLILTCLVVVALARPKHPINHQGLSPEVLNENLLRFVVAPFPEVFRKENINELSKDIGSESTEDQAMEDAKQMKAGSSSSSEEIVPNSAQKYIQKEDVPSERYLGYLEQLLRLKKYNVPQLEIVPKSAEEQLHSMKEGNPAHQKQPMIAVNQELAYFYPQLFRQFYQLDAYPSGAWYYLPLGTQYTDAPSFSDIPNPIGSENSGKTTMPLW

SEQ ID NO: 391 - protein, 214 aa, Capra hircus
MKLLILTCLVAVALARPKHPINHQGLSPEVPNENLLRFVVAPFPEVFRKENINELSKDIGSESTEDQAMEDAKQMKAGSSSSSEEIVPNSAEQKYIQKEDVPSERYLGYLEQLLKLKKYNVPQLEIVPKSAEEQLHSMKEGNPAHQKQPMIAVNQELAYFYPQLFRQFYQLDAYPSGAWYYLPLGTQYTDAPSFSDIPNPIGSENSGKTAMPLW

SEQ ID NO: 392 - protein, 214 aa, Capra hircus
MKLLILTCLVAVALARPKHPINHQGLSPEVPNENLLRFVVAPFPEVFRKENINELSKDIGSESTEDQAMEDAKQMKAGSSSSSEEIVPNSAEQKYIQKEDVPSERYLGYLEQLLKLKKYNVPQLEIVPKSAEEQLHSMKEGNPAHQKQPMIAVNQELAYFYPQLFRQFYQLDAYPSGAWYYLPLGTQYTDAPSFSDIPNPIGSENSGKAAMPLW

SEQ ID NO: 393 - protein, 213 aa, Capra hircus
MKLLILTCLVAVALARPKHPINHQGLSPEVPNENLLRFVVAPFPEVFRKENINELSKDIGSESTEDQAMEDAKQMKAGSSSSSEEIVPNSAEKYIQKEDVPSERYLGYLEQLLRLKKYNVPQLETVPNSAEEQLHSMKEGNPAHQKQPMIAVNQELAYFYPQLFRQFYQLDAYPSGAWYYLPLGTQYTDAPSFSDIPNPIGSENSGKTTMPLW

SEQ ID NO: 394 - protein, 212 aa, Capra hircus
KLLILTCLVAVALARPKHPINHQGLSPEVPNENLLRFVVAPFPEVFRKENINELSKDIGSESTEDQAMEDAKQMKAGSSSSSEEIVPNSAEKYIQKEDVPSERYLGYLEQLLRLKKYNVPQLEIVPKSAEEQLHSMKEGNPAHQKQPMIAVNQELAYFYPQLFRQFYQLDAYPSGAWYYLPLGTQYTDAPSFSDIPNPIGSENSGKTTMPLW

SEQ ID NO: 395 - protein, 214 aa, Capra hircus
MKLLILTCLVAVALARPKHPINIQGLSPEVPNENLLRFVVAPFPEVFRKENINELSKDIGSESTEDQAMEDAKQMKAGSSSSSEEIVPNSAEQKYIQKEDVPSERYLGYLEQLLKLKKYNVPQLEIVPKSAEEQLHSMKEGNPAHQKQPMIAVNQELAYFYPQLFRQFYQLDAYPSGAWYYLPLGTQYTDAPSFSDIPNPIGSENSGKTAMPLW

SEQ ID NO: 396 - protein, 215 aa, Capra hircus
MKLLILTCLVAVALARPKHPINHQGLSPVSEVLNENLLRFVVAPFPEVFRRENINELSKDIGSESTEDQAMEDAKQMKAGSSSSSEEIVPNSAQKYIQKEGVPSERYLGYLEQLLRLKKYNVPQLEIVPKSAEEQLHSMKEGNPAHQKQPMIAVNQELAYFYPQLFRQFYQLDAYPSGAWYYLPLGTQYTDAPSFSDIPNPIGSENSGKTTMPLW

SEQ ID NO: 397 - protein, 213 aa, Capra hircus
MKLLILTCLVAVALARPKHPINIQGLSPEVPNENLLRFVVAPFPEVFRKENINELSKDIGSESTEDQAMEDAKQMKAGSSSSSEEIVPNSAEKYIQKEDVPSERYLGYLEQLLKLKKYNVPQLEIVPKSAEEQLHSMKEGNPAHQKQPMIAVNQELAYFYPQLFRQFYQLDAYPSGAWYYLPLGTQYTDAPSFSDIPNPIGSENSGKTAMPLW

SEQ ID NO: 398 - protein, 206 aa, Capra hircus
MKLLILTCLVAVALARPKHPINHQGLSPEVLNENLLRFVVAPFPEVFRKENINELSKDIGSESTEDQAMEDAKQMKAGSSSSSEEIVPNSAQQKYIQKEDVPSERYLGYLEQLLRLKKYNVPQLEQLHSMKEGNPAHQKQPMIAVNQELAYFYPQLFRQFYQLDAYPSGAWYYLPLGTQYTDAPSFSDIPNPIGSENSGKTTMPLW

SEQ ID NO: 399 - protein, 203 aa, Capra hircus
MKLLILTCLVAVALARPEVLNENLLRFVVAPFPEVFRKENINELSKDIGSESTEDQAMEDAKQMKAGSSSSSEEIVPNSAQQKYIQKEDVPSERYLGYLEQLLRLKKYNVPQLEIVPKSAEEQLHSMKEGNPAHQKQPMIAVNQELAYFYPQLFRQFYQLDAYPSGAWYYLPLGTQYTDAPSFSDIPNPIGSENSGKTTMPLW

SEQ ID NO: 400 - protein, 206 aa, Capra hircus
MKLLILTCLVAVALARPKHPINHQGLSPEVLNENLLRFVVAPFPEVFRKENINELSKDIGSESTEDQAMEDAKQMKAGSSSSSEEIVPNSAQQKYIQKEDVPSERYLGYLEQLLRLKKYNVPQLEIVPKSAEEQLHSMKEGNPAHQKQPMIAVNQLFRQFYQLDAYPSGAWYYLPLGTQYTDAPSFSDIPNPIGSENSGKTTMPLW

SEQ ID NO: 401 - protein, 198 aa, Capra hircus
MKLLILTCLVAVALARPKHPINHQGLSPEVLNENLLRFVVAPFPEVFRKENINELSKDIGSESTEDQAMEDAKQMKAGSSSSSEEIVPNSAQQKYIQKEDVPSERYLGYLEQLLRLKKYNVPQLEQLHSMKEGNPAHQKQPMIAVNQLFRQFYQLDAYPSGAWYYLPLGTQYTDAPSFSDIPNPIGSENSGKTTMPLW

SEQ ID NO: 402 - protein, 177 aa, Capra hircus
MKLLILTCLVAVALARPKHPINHQGLSPEVLNENLLRFVVAPFPEVFRKENINELSKDIGSESTEDQAMEDAKEQLLRLKKYNVPQLEIVPKSAEEQLHSMKEGNPAHQKQPMIAVNQELAYFYPQLFRQFYQLDAYPSGAWYYLPLGTQYTDAPSFSDIPNPIGSENSGKTTMPLW

SEQ ID NO: 403 - protein, 214 aa, Ovis aries
MKLLILTCLVAVALARPKHPIKHQGLSPEVLNENLLRFVVAPFPEVFRKENINELSKDIGSESIEDQAMEDAKQMKAGSSSSSEEIVPNSAEQKYIQKEDVPSERYLGYLEQLLRLKKYNVPQLEIVPKSAEEQLHSMKEGNPAHQKQPMIAVNQELAYFYPQLFRQFYQLDAYPSGAWYYLPLGTQYTDAPSFSDIPNPIGSENSGKTTMPLW

SEQ ID NO: 404 - protein, 214 aa, Ovis aries
MKLLILTCLVAVALARPKHPIKHQGLSSEVLNENLLRFVVAPFPEVFRKENINELSKDIGSESIEDQAMEDAKQMKAGSSSSSEEIVPNSAEQKYIQKEDVPSERYLGYLEQLLRLKKYNVPQLEIVPKSAEEQLHSMKEGNPAHQKQPMIAVNQELAYFYPQLFRQFYQLDAYPSGAWYYLPLGTQYTDAPSFSDIPNPIGSENSGKITMPLW

SEQ ID NO: 405 - protein, 199 aa, Ovis aries; identical to SEQ ID NO: 100

SEQ ID NO: 406 - protein, 214 aa, Ovis aries
MKLLILTCLVAVALARPKHPIKHQGLSPEVLNENLLRFVVAPFPEVFRKENINELSKDIGSESIEDQAMEDAKQMKAGSSSSSEEIVPNSAEQKYIQKEDVPSERYLGYLEQLLRLKKYNVPQLEIVPKSAEEQLHSMKEGNPAHQKQPMIAVNQELAYFYPQLFRQFYQLDAYPSGAWYYLPLGTQYTDAPSFSDIPNPIGSENSGKITMPLW

SEQ ID NO: 407 - protein, 206 aa, Ovis aries
MKLLILTCLVAVALARPKHPIKHQGLSPEVLNENLLRFVVAPFPEVFRKENINELSKDQAMEDAKQMKAGSSSSSEEIVPNSAEQKYIQKEDVPSERYLGYLEQLLRLKKYNVPQLEIVPKSAEEQLHSMKEGNPAHQKQPMIAVNQELAYFYPQLFRQFYQLDAYPSGAWYYLPLGTQYTDAPSFSDIPNPIGSENSGKTTMPLW

SEQ ID NO: 408 - protein, 206 aa, Ovis aries
MKLLILTCLVAVALARPKHPIKHQGLSPEVLNENLLRFVVAPFPEVFRKENINELSKDIGSESIEQMKAGSSSSSEEIVPNSAEQKYIQKEDVPSERYLGYLEQLLRLKKYNVPQLEIVPKSAEEQLHSMKEGNPAHQKQPMIAVNQELAYFYPQLFRQFYQLDAYPSGAWYYLPLGTQYTDAPSFSDIPNPIGSENSGKTTMPLW

SEQ ID NO: 409 - protein, 206 aa, Ovis aries
MKLLILTCLVAVALARPKHPIKHQGLSPEVLNENLLRFVVAPFPEVFRKENINELSKDQAMEDAKQMKAGSSSSSEEIVPNSAEQKYIQKEDVPSERYLGYLEQLLRLKKYNVPQLEIVPKSAEEQLHSMKEGNPAHQKQPMIAVNQELAYFYPQLFRQFYQLDAYPSGAWYYLPLGTQYTDAPSFSDIPNPIGSENSGKITMPLW

SEQ ID NO: 410 - protein, 206 aa, Ovis aries
MKLLILTCLVAVALARPKHPIKHQGLSPEVLNENLLRFVVAPFPEVFRKENINELSKDIGSESIEQMKAGSSSSSEEIVPNSAEQKYIQKEDVPSERYLGYLEQLLRLKKYNVPQLEIVPKSAEEQLHSMKEGNPAHQKQPMIAVNQELAYFYPQLFRQFYQLDAYPSGAWYYLPLGTQYTDAPSFSDIPNPIGSENSGKITMPLW

SEQ ID NO: 411 - protein, 191 aa, Ovis aries
RPKHPIKHQGLSSEVLNENLLRFVVAPFPEVFRKENINELSKDIGSESIEDQAMEDAKQMKAGSSSSSEEIVPNSAEQKYIQKEDVPSERYLGYLEQLLRLKKYNVPQLEIVPKSAEEQLHSMKEGNPAHQKQPMIAVNQLFRQFYQLDAYPSGAWYYLPLGTQYTDAPSFSDIPNPIGSENSGKITMPLW

SEQ ID NO: 412 - protein, 206 aa, Ovis aries
MKLLILTCLVAVALARPKHPIKHQGLSPEVLNENLLRFVVAPFPEVFRKENINELSKDIGSESIEDQAMEDAKQMKAGSSSSSEEIVPNSAEQKYIQKEDVPSERYLGYLEQLLRLKKYNVPQLEIVPKSAEEQLHSMKEGNPAHQKQPMIAVNQLFRQFYQLDAYPSGAWYYLPLGTQYTDAPSFSDIPNPIGSENSGKITMPLW

SEQ ID NO: 413 - protein, 214 aa, Bubalus bubalis
MKLLILTCLVAVALARPKQPIKHQGLPQGVLNENLLRFFVAPFPEVFGKEKVNELSTDIGSESTEDQAMEDIKQMEAESISSSEEIVPISVEQKHIQKEDVPSERYLGYLEQLLRLKKYNVPQLEIVPNLAEEQLHSMKEGIHAQQKEPMIGVNQELAYFYPQLFRQFYQLDAYPSGAWYYVPLGTQYPDAPSFSDIPNPIGSENSEKTTMPLW

SEQ ID NO: 414 - protein, 214 aa, Bubalus bubalis
MKLLVLTCLVAVALARPKQPIKHQGLPQGVLNENLLRFFVAPFPEVFGKEKVNELSTDIGSESTEDQAMEDIKQMEAESISSSEEIVPISVEQKHIQKEDVPSERYLGYLEQLLRLKKYNVPQLEIVPNLAEEQLHSMKEGIHAQQKEPMIGVNQELAYFYPQLFRQFYQLDAYPSGAWYYVPLGTQYPDAPSFSDIPNPIGSENSGKTTMPLW

SEQ ID NO: 415 - protein, 214 aa, Bubalus bubalis
MKLLILTCLVAVALARPKQPIKHQGLPQGVLNENLLRFFVAPFPEVFGKEKVNELSTDIGSESTEDQAMEDIKQMEAESISSSEEIVPISVEQKHIQKEDVPSERYLGYLEQLLRLKKYNVPQLEIVPNLAEEQLHSMKEGIHAQQKEPMIGVNQELAYFYPQLFRQFYQLDAYPSGAWYYVPLGTQYPDAPSFSDIPNPIGSENSGKTTMPLW

SEQ ID NO: 416 - protein, 214 aa, Bubalus bubalis
MKLLILTCLVAVALARPKQPIKHQGLPQGVLNENLLRFFVAPFPEVFGKEKVNELSTDIGSESTEDQAMEDIKQMEAESISSSEEIVPISVEQKHIQKEDVPSERYLGYLEQLLRLKKYNVPQLEIVPNLAEEQLHSMKEGIHAQQKEPMIGVNQELAYFYPQLFRQFYQLDAYPSGAWYYVPLGTQYPDAPLFSDIPNPIGSENSGKTTMPLW

SEQ ID NO: 417 - protein, 214 aa, Bubalus bubalis
MKLLILTCLVAVALARPKQPIKHQGLPQGVLNENLLRFFVAPFPEVFGKEKVNELSTDVGSESTEDQAMEDIKQMEAESISSSEEIVPISVEQKHIQKEDVPSERYLGYLEQLLRLKKYNVPQLEIVPNLAEEQLHSMKEGIHAQQKEPMIGVNQELAYFYPQLFRQFYQLDAYPSGAWYYVPLGTQYPDAPSLSDIPNPIGSENSGKTTMPLW

SEQ ID NO: 418 - protein, 215 aa, Bubalus bubalis
MKLLILTCLVAVALARPKQPIKHQGLPQGVLNENLLRFFVAPFPEVFGKEKVNELSTDIGSESTEDQAMEDIKQMEAESISSSEEIVPISVEQKHIQKEDVPSERYLGYLEQLLRLKKYNVPQLEIVPNLAEEQLHSMKEGIHAQQKEPMIGVNQELAYFYPQLFRQFYQLDAYPSGAWYYVPLGTQYPDAPLFSDIPNPIGSENSGKTTMPLWW

SEQ ID NO: 419 - protein, 214 aa, Bubalus bubalis
MKLLILTCLVAVALARPKQPIKHQGLPQGVLNENLLRFFVAPFPEMFGKEKVNELSTDVGSESTEDQAMEDIKQMEAESISSSEEIVPISVEQKHIQKEDVPSERYLGYLEQLLRLKKYNVPQLEIVPNLAEEQLHSMKEGIHAQQKEPMIGVNQELAYFYPQLFRQFYQLDAYPSGAWYYVPLGTQYPDAPLFSDIPNPIGSENSGKTTMPLW

SEQ ID NO: 420 - protein, 214 aa, Bubalus bubalis
MKLLILTCLVAVALARPKQPIKHQGLPQGVLNENLLRFFVAPFPEVFGKEKVNELSTDIGSESTEDQAMEDIKQMEAESISSSEEIVPISVEKHIQKEDVPSERYLGYLEQLLRLKKYNVPQLEIVPNLAEEQLHSMKEGIHAQQKEPMIGVNQELAYFYPQLFRQFYQLDAYPSGAWYYVPLGTQYPDAPLFSDIPNPIGSENSGKTTMPLWW

SEQ ID NO: 421 - protein, 206 aa, Bubalus bubalis
MKLLILTCLVAVALARPKQPIKHQGLPQGVLNENLLRFFVAPFPEMFGKDVGSESTEDQAMEDIKQMEAESISSSEEIVPISVEQKHIQKEDVPSERYLGYLEQLLRLKKYNVPQLEIVPNLAEEQLHSMKEGIHAQQKEPMIGVNQELAYFYPQLFRQFYQLDAYPSGAWYYVPLGTQYPDAPSFSDIPNPIGSENSGKTTMPLW

SEQ ID NO: 422 - protein, 206 aa, Bubalus bubalis
MKLLILTCLVAVALARPKQPIKHQGLPQGVLNENLLRFFVAPFPEVFGKDIGSESTEDQAMEDIKQMEAESISSSEEIVPISVEQKHIQKEDVPSERYLGYLEQLLRLKKYNVPQLEIVPNLAEEQLHSMKEGIHAQQKEPMIGVNQELAYFYPQLFRQFYQLDAYPSGAWYYVPLGTQYPDAPLFSDIPNPIGSENSGKTTMPLW

SEQ ID NO: 423 - protein, 207 aa, Bubalus bubalis
MKLLILTCLVAVALARPKQPIKHQGLPQGVLNENLLRFFVAPFPEVFGKDIGSESTEDQAMEDIKQMEAESISSSEEIVPISVEQKHIQKEDVPSERYLGYLEQLLRLKKYNVPQLEIVPNLAEEQLHSMKEGIHAQQKEPMIGVNQELAYFYPQLFRQFYQLDAYPSGAWYYVPLGTQYPDAPLFSDIPNPIGSENSGKTTMPLWW

SEQ ID NO: 424 - protein, 207 aa, Bubalus bubalis
MKLLILTCLVAVALARPKQPIKHQGLPQGVLNENLLRFFVAPFPEVFGKEKVNELSTDIGSESTEDQAMEDIKQMEAESISSSEEIVPISVEQKHIQKEDVPSERYLGYLEQLLRLKKYNVPQLEIVPNLAEEQLHSMKEGIHAQQKEPMIGVNQLFRQFYQLDAYPSGAWYYVPLGTQYPDAPLFSDIPNPIGSENSGKTTMPLWW

SEQ ID NO: 425 - protein, 207 aa, Bubalus bubalis
MKLLILTCLVAVALARPKQPIKHQGLPQGVLNENLLRFFVAPFPEVFGKEKVNELSTDQAMEDIKQMEAESISSSEEIVPISVEQKHIQKEDVPSERYLGYLEQLLRLKKYNVPQLEIVPNLAEEQLHSMKEGIHAQQKEPMIGVNQELAYFYPQLFRQFYQLDAYPSGAWYYVPLGTQYPDAPLFSDIPNPIGSENSGKTTMPLWW

SEQ ID NO: 426 - protein, 206 aa, Bubalus bubalis
MKLLILTCLVAVALARPKQPIKHQGLPQGVLNENLLRFFVAPFPEVFGKEKVNELSTDQAMEDIKQMEAESISSSEEIVPISVEKHIQKEDVPSERYLGYLEQLLRLKKYNVPQLEIVPNLAEEQLHSMKEGIHAQQKEPMIGVNQELAYFYPQLFRQFYQLDAYPSGAWYYVPLGTQYPDAPLFSDIPNPIGSENSGKTTMPLWW

SEQ ID NO: 427 - protein, 202 aa, Bubalus bubalis
MKLLILTCLVAVALARPKQPIKHQGLPQPFPEVFGKEKVNELSTDIGSESTEDQAMEDIKQMEAESISSSEEIVPISVEQKHIQKEDVPSERYLGYLEQLLRLKKYNVPQLEIVPNLAEEQLHSMKEGIHAQQKEPMIGVNQELAYFYPQLFRQFYQLDAYPSGAWYYVPLGTQYPDAPLFSDIPNPIGSENSGKTTMPLWW

SEQ ID NO: 428 - protein, 194 aa, Bubalus bubalis
MKLLILTCLVAVALARPKQPIKHQGLPQPFPEVFGKDIGSESTEDQAMEDIKQMEAESISSSEEIVPISVEQKHIQKEDVPSERYLGYLEQLLRLKKYNVPQLEIVPNLAEEQLHSMKEGIHAQQKEPMIGVNQELAYFYPQLFRQFYQLDAYPSGAWYYVPLGTQYPDAPLFSDIPNPIGSENSGKTTMPLWW

SEQ ID NO: 429 - protein, 194 aa, Bubalus bubalis
MKLLILTCLVAVALARPKQPIKHQGLPQPFPEVFGKEKVNELSTDQAMEDIKQMEAESISSSEEIVPISVEQKHIQKEDVPSERYLGYLEQLLRLKKYNVPQLEIVPNLAEEQLHSMKEGIHAQQKEPMIGVNQELAYFYPQLFRQFYQLDAYPSGAWYYVPLGTQYPDAPLFSDIPNPIGSENSGKTTMPLWW

SEQ ID NO: 430 - protein, 178 aa, Bubalus bubalis
MKLLILTCLVAVALARPKQPIKHQGLPQGVLNENLLRFFVAPFPEVFGKEKVNELSTDIGSESTEDQAMEDIKEQLLRLKKYNVPQLEIVPNLAEEQLHSMKEGIHAQQKEPMIGVNQELAYFYPQLFRQFYQLDAYPSGAWYYVPLGTQYPDAPLFSDIPNPIGSENSGKTTMPLWW

SEQ ID NO: 431 - protein, 214 aa, Bos mutus
MKLLILTCLVAVALARPKHPIKHQGLPQEVLNENLLRFFVAPFPEVFGKEKVNELSKDIGSESTEDQAMEDIKKMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSGKTTMPLW

SEQ ID NO: 432 - protein, 214 aa, Bos indicus
  VARIANT 99: note = X is any amino acid
MKLLILTCLVAVALARPKHPIKHQGLPQEVLNENLLRFFVAPFPEVFGKEKVNELSKDIGSESTEDQAMEDIKQMEAESISSSEEIVPNSVEQKHIQKXDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSGKTTMPLW

SEQ ID NO: 433 - protein, 205 aa, Jeotgalicoccus coquinae
MAVALARPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 434 - protein, 146 aa, Jeotgalicoccus coquinae
MEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 435 - protein, 214 aa, Bison bison
MKLLILTCLVAVALARPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSGKTTMPLW

SEQ ID NO: 436 - protein, 214 aa, Bos grunniens
MRLLILTCLVAVALARPKHPIKHQGLPQEVLNENLLRFFVAPFPEVFGKEKVNELSKDIGSESTEDQAMEDIKQMEAESISSSEEIVPNSVEQKHIQKEDVPSEHYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSGKTTMPLW

SEQ ID NO: 437 - protein, 214 aa, Jeotgalicoccus aerolatus
MKLLILTCLVAVALARPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 438 - protein, 196 aa, Jeotgalicoccus aerolatus
LVAVALARPKQPIKHQGLPQGVLNENLLRFFVAPFPEVFGKEKVNELSTDIGSESTEDQAMEDIKQMEAESISSSEEIVPISVEQKHIQKEDVPSERYLGYLEQLLRLKKYNVPQLEIVPNLAEEQLHSMKEGIHAQQKEPMIGVNQELAYFYPQLFRQFYQLDAYPSGAWYYVPLGTQYPDAPSFSDIPNPIGSE

SEQ ID NO: 439 - protein, 229 aa, Muntiacus muntjak
KHPIGHQGLPQEVLNENLLRLFVVPFPEVFGKESINELSKDTESESTEDHAMEDTKQVESGSSSSSEEIVSNIAEQKQVQKEDVPSERYLGYLEQLLKLQKYNLPQLEQKIRSPRELLDYFPPDMKHRFFVLPNYSKYNTELNIFSKELLHSMKEGIHAQQKKPMKGVSQELAYFYPQLFRQFYQLDAYPSGAWYYLPLGIQYTDVPSFSDIPNPIGSENSGKATMPLW

SEQ ID NO: 440 - protein, 229 aa, Muntiacus reevesi
KHPIGHQGLPQEVLNENLLRLFVVPFPEVFGKESISELSKDTESESTEDHAMEDTKQVESGSSSSSEEIVSNIAEQKQVQKEDVPSERYLGYLEQLLKLQKYNLPQLEQKIRSPRELLDYFPPDMKHRFFVLPNYSKYNTELNIFSKELLHSMKEGIHAQQKKPMKGVSQELAYFYPQLFRQFYQLDAYPSGAWYYLPLGIQYTDAPSFSDIPNPIGSENSGKATMPLW

SEQ ID NO: 441 - protein, 222 aa, Bos taurus
MKFFIFTCLLAVALAKNTMEHVSSSEESIISQETYKQEKNMDINPSKENLCSTFCKEVVRNANEEEYSIGSSSEESAEVATEEVKITVDDKHYQKALNEINQFYRKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMKPWIQPKTKVIPYVRYL

SEQ ID NO: 442 - protein, 213 aa, Bos taurus
MKFFIFTCLLAVALAKNTMEHVSSSEESIISQETYKQEKNMAINPSKENLCSTFCKEVVRNANEEEYSIGSSSEESAEVATEEVKITVDDKHYQKALNEINQFYQKFPQYLQYLYQGPIVLNPWDQVKRNAVPITPTLNREQLSTSEESTEVFTKKTKLTEEEKNRLNFLKKISQRYQKFALPQYLKTVYQHQKAMKPWIQPKTKVIPYVRYL

SEQ ID NO: 443 - protein, 213 aa, Bos taurus
MKFFIFTCLLAVALAKNTMEHVSSSEESIISQETYKQEKNMAINPSKEVVRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMKPWIQPKTKVIPYVRYL

SEQ ID NO: 444 - protein, 223 aa, Capra hircus
MKFFIFTCLLAVALAKHKMEHVSSSEEPINILQEIYKQEKNMAIHPRKEKLCTTSCEEVVRNANEEEYSIRSSSEESAEVAPEEIKITVDDKHYQKALNEINQFYQKFPQYLQYPYQGPIVLNPWDQVKRNAGPFTPTVNREQLSTSEENSKKTIDMESTEVFTKKTKLTEEEKNRLNFLKKISQYYQKFAWPQYLKTVDQHQKAMKPWTQPKTNAIPYVRYL

SEQ ID NO: 445 - protein, 223 aa, Capra hircus
MKFFIFTCLLAVALAKHKMEHVSSSEEPINIFQEIYKQEKNMAIHPRKEKLCTTSCEEVVRNANEEEYSIRSSSEESAEVAPEEIKITVDDKHYQKALNEINQFYQKFPQYLQYPYQGPIVLNPWDQVKRNAGPFTPTVNREQLSTSEENSKKTIDMESTEVFTKKTKLTEEEKNRLNFLKKISQYYQKFAWPQYLKTVDQHQKAMKPWTQPKTNAIPYVRYL

SEQ ID NO: 446 - protein, 223 aa, Capra hircus
MKFFIFTCLLAVALAKHKMEHISSSEEPINIFQEIYKQEKNMAIHPRKEKLCTTSCEEVVRNANEEEYSIRSSSEESAEVAPEEIKITVDDKHYQKALNEINQFYQKFPQYLQYPYQGPIVLNPWDQVKRNAGPFTPTVNREQLSTSEENSKKTIDMESTEVFTKKTKLTEEEKNRLNFLKKISQYYQKFAWPQYLKTVDQHQKAMKPWTQPKTNAIPYVRYL

SEQ ID NO: 447 - protein, 223 aa, Capra hircus
MKFFIFTCLLAVALAKHKMEHVSSSEEPINIFQEIYKQEKNMAIHPRKEKLCTTSCEEVVRNANEEEYSIRSSSEESAKVAPEEIKITVDDKHYQKALNEINQFYQKFPQYLQYPYQGPIVLNPWDQVKRNAGPFTPTVNREQLSTSEENSKKTIDMESTEVFTKKTKLTEEEKNRLNFLKKISQYYQKFAWPQYLKTVDQHQKAMKPWTQPKTNAIPYVRYL

SEQ ID NO: 448 - protein, 223 aa, Capra hircus
MKFFIFTCLLAVALAKHKMEHVSSSEEPINIFQEIYKQEKNMAIHPRKEKLCTTSCEEVVRNANEEEYSIRSSSEESAEVAPEEIKITVDDKHYQKALNEINQFYQKFPQYLQYPYQGPIVLNPWDQVKRNAGPFTPTVNREQLSTSEENSKKTIDMESTEVFTKKTKLTEEEKNRLNFLKIISQYYQKFAWPQYLKTVDQHQKAMKPWTQPKTNAIPYVRYL

SEQ ID NO: 449 - protein, 223 aa, Capra hircus
MKFFIFTCLLAVALATHKMEHVSSSEEPINIFQEIYKQEKNMAIHPRKEKLCTTSCEEVVRNANEEEYSIRSSSEESAKVAPEEIKITVDDKHYQKALNEINQFYQKFPQYLQYPYQGPIVLNPWDQVKRNAGPFTPTVNREQLSTSEENSKKTIDMESTEVFTKKTKLTEEEKNRLNFLKKISQYYQKFAWPQYLKTVDQHQKAMKPWTQPKTNAIPYVRYL

SEQ ID NO: 450 - protein, 223 aa, Capra hircus
MKFFIFTCLLAVALAKHKMEHVSSSEEPINIFQEIYKQEKNMAIHPRKEKLCTTSCEEVVRNANEEEYSIRSSSEESAEVAPEEIKITVDDKHYQKALNEINQFYQKFPQYLQYPYQGPIVLNPWDQVKRNAGAFTPTVNREQLSTSEENSKKTIDMESTEVFTKKTKLTEEEKNRLNFLKKISQYYQKFAWPQYLKTVDQHQKAMKPWTQPKTNAIPYVRYL

SEQ ID NO: 451 - protein, 223 aa, Capra hircus
MKFFIFTCLLAVALAKHKMEHVSSSEEPINIFQEIYKQEKNMAIHPRKEKLCTTSCEEVVRNANEEEYSIRSSSEESAEVAPEEIKITVDDKHYQKALNEINQFYQKFPQYLQYPYQGPIVLNPWDQVKRNAGPFTPTVNREQLSTSEENSKKTIDMESTEVFTKKAKLTEEEKNRLNFLKIISQYYQKFAWPQYLKTVEQHQKAMKPWTQPKTNAIPYVRYL

SEQ ID NO: 452 - protein, 222 aa, Capra hircus
MKFFIFTCLLAVALAKHMEHVSSSEEPINIFQEIYKQEKNMAIHPRKEKLCTTSCEEVVRNANEEEYSIRSSSEESAEVAPEEIKITVDDKHYQKALNEINQFYQKFPQYLQYPYQGPIVLNPWDQVKRNAGAFTPTVNREQLSTSEENSKKTIDMESTEVFTKKTKLTEEEKNRLNFLKKISQYYQKFAWPQYLKTVDQHQKAMKPWTQPKTNAIPYVRYL

SEQ ID NO: 453 - protein, 223 aa, Capra hircus
MKFFIFTCLLAVALAKHKMEHVSSSEEPINIFQEIYKQEKNMAIRPRKEKLCTTSCEEVVRNANEEEYSIRSSSEESAEVAPEEIKITVDDKHYQKALNEINQFYQKFPQYLQYPYQGPIVLNPWDQVKRNAGPFTPTVNREQLSTSEENSKKTIDMESTEVFTKKTKLTEEEKNRLNFLKKISQYYQKFAWPQYLKTVDQHQKAMKPRTQPKTNAIPYVRYL

SEQ ID NO: 454 - protein, 223 aa, Capra hircus
MKFFIFTCLLAVALAKHKMEHVSSSEEPINIFQEIYKQEKNMAIHPRKEKLCTTSCEEVVRNANEEEYSIRSSSEESAEVAPEEIKITVDDKHYQKALNEINQFYQKFPQYLQYPYQGPIVLNPWDQVKRNAGPFTPTVNREQLSTSEENSKKTIDMESTEVFTKKTKLTEEEKNRLNFLKIISQYYQKFAWPQYLKTVDQHQKAMKRWTQPKTNAIPYVRYL

SEQ ID NO: 455 - protein, 223 aa, Capra hircus
MKFFIFTCLLAVALAKHKMEHVSSSEEPINIFQEIYKQEKNMAIHPRKEKLCTTSCEEVVRNANEEEYSIRSSSEESAEVAPEEIKITVDDKHYQKALNEINQFYQKFPQYLQYPYQGPIVLNPWDQVKRNAGPFTPTVNREQLSTSEENSKKTIDMESTKVFTKKTKLTEEEKNRLNFLKIISQYYQKFAWPQYLKTVDQHQKAMKRWTQPKTNAIPYVRYL

SEQ ID NO: 456 - protein, 215 aa, Capra hircus
MKFFIFTCLLAVALAKHKMEHVSSSEEPINIFQEIYKQEKNMAIHPRKEKLCTTSCEEVVRNANEEEYSIRSSSEEIKITVDDKHYQKALNEINQFYQKFPQYLQYPYQGPIVLNPWDQVKRNAGPFTPTVNREQLSTSEENSKKTIDMESTEVFTKKTKLTEEEKNRLNFLKKISQYYQKFAWPQYLKTVDQHQKAMKPWTQPKTNAIPYVRYL

SEQ ID NO: 457 - protein, 214 aa, Capra hircus
MKFFIFTCLLAVALAKHEPINIFQEIYKQEKNMAIHPRKEKLCTTSCEEVVRNANEEEYSIRSSSEESAEVAPEEIKITVDDKHYQKALNEINQFYQKFPQYLQYPYQGPIVLNPWDQVKRNAGAFTPTVNREQLSTSEENSKKTIDMESTEVFTKKTKLTEEEKNRLNFLKKISQYYQKFAWPQYLKTVDQHQKAMKPWTQPKTNAIPYVRYL

SEQ ID NO: 458 - protein, 214 aa, Capra hircus
MKFFIFTCLLAVALAKHKMEHVSSSEEPINIFQEIYKQEKNMAIHPRKEVVRNANEEEYSIRSSSEESAEVAPEEIKITVDDKHYQKALNEINQFYQKFPQYLQYPYQGPIVLNPWDQVKRNAGPFTPTVNREQLSTSEENSKKTIDMESTEVFTKKTKLTEEEKNRLNFLKKISQYYQKFAWPQYLKTVDQHQKAMKPWTQPKTNAIPYVRYL

SEQ ID NO: 459 - protein, 214 aa, Capra hircus
MKFFIFTCLLAVALAKHEPINIFQEIYKQEKNMAIHPRKEKLCTTSCEEVVRNANEEEYSIRSSSEESAKVAPEEIKITVDDKHYQKALNEINQFYQKFPQYLQYPYQGPIVLNPWDQVKRNAGPFTPTVNREQLSTSEENSKKTIDMESTEVFTKKTKLTEEEKNRLNFLKKISQYYQKFAWPQYLKTVDQYQKAMKPWTQPKTNAIPYVRYL

SEQ ID NO: 460 - protein, 215 aa, Capra hircus
MKFFIFTCLLAVALAKHKMEHVSSSEEPINIFQEIYKQEKNMAIHPRKEKLCTTSCEEVVRNANEEEYSIRSSSEEIKITVDDKHYQKALNEINQFYQKFPQYLQYPYQGPIVLNPWDQVKRNAGAFTPTVNREQLSTSEENSKKTIDMESTEVFTKKTKLTEEEKNRLNFLKKISQYYQKFAWPQYLKTVDQHQKAMKPWTQPKTNAIPYVRYL

SEQ ID NO: 461 - protein, 214 aa, Capra hircus
MKFFIFTCLLAVALAKHKMEHVSSSEEPINIFQEIYKQEKNMAIHPRKEVVRNANEEEYSIRSSSEESAEVAPEEIKITVDDKHYQKALNEINQFYQKFPQYLQYPYQGPIVLNPWDQVKRNAGPFTPTVNREQLSTSEENSKKTIDMESTEVFTKKTKLTEEEKNRLNFLKIISQYYQKFAWPQYLKTVDQHQKAMKPWTQPKTNAIPYVRYL

SEQ ID NO: 462 - protein, 214 aa, Capra hircus
MKFFIFTCLLAVALAKHKMEHVSSSEEPINIFQEIYKQEKNMAIHPRKEVVRNANEEEYSIRSSSEESAEVAPEEIKITVDDKHYQKALNEINQFYQKFPQYLQYPYQGPIVLNPWDQVKRNAGAFTPTVNREQLSTSEENSKKTIDMESTEVFTKKTKLTEEEKNRLNFLKKISQYYQKFAWPQYLKTVDQHQKAMKPWTQPKTNAIPYVRYL

SEQ ID NO: 463 - protein, 214 aa, Capra hircus
MKFFIFTCLLAVALAKHKMEHVSSSEEPINIFQEIYKQEKNMAIHPRKEVVRNANEEEYSIRSSSEESAKVAPEEIKITVDDKHYQKALNEINQFYQKFLQYLQYPYQGPIVLNPWDQVKRNAGPFTPTVNREQLSTSEENSKKTIDMESTEVFTKKTKLTEEEKNRLNFLKKISQYYQKFAWPQYLKTVDQHQKAMKPWTQPKTNAIPYVRYL

SEQ ID NO: 464 - protein, 214 aa, Capra hircus
MKFFIFTCLLAVALAKHKMEHVSSSEEPINIFQEIYKQEKNMAIHPRKEVVRNANEEEYSIRSSSEESAEVAPKEIKITVDDKHYQKALNEINQFYQKFPQYLQYPYQGPIVLNPWDQVKRNAGPFTPTVNREQLSTSEENSKKTIDMESTEVFTKKTKLTEEEKNRLNFLKIISQYYQKFAWPQYLKTVDQHQKAMKAWTQPKTNAIPYVRYL

SEQ ID NO: 465 - protein, 214 aa, Capra hircus
MKFFIFTCLLAVALAKHKMEHVSSSEEPINIFQEIYKQEKNMAIHPRKEKLCTTSCEEVVRNANEEEYSIRSSSEESAEVAPEEIKITVDDKHYQKALNEINQFYQKFPQYLQYPYQGPIVLNPWDQVKRNAGPFTPTVNREQLSTSEESTEVFTKKTKLTEEEKNRLNFLKIISRYYQKFAWPQYLKTVDQHQKAMKPWTQPKTNAIPYVRYL

SEQ ID NO: 466 - protein, 205 aa, Capra hircus
MKFFIFTCLLAVALAKHEPINIFQEIYKQEKNMAIHPRKEVVRNANEEEYSIRSSSEESAEVAPEEIKITVDDKHYQKALNEINQFYQKFPQYLQYPYQGPIVLNPWDQVKRNAGAFTPTVNREQLSTSEENSKKTIDMESTEVFTKKTKLTEEEKNRLNFLKKISQYYQKFAWPQYLKTVDQHQKAMKPWTQPKTNAIPYVRYL

SEQ ID NO: 467 - protein, 206 aa, Capra hircus
MKFFIFTCLLAVALAKHKMEHVSSSEEPINIFQEIYKQEKNMAIHPRKEVVRNANEEEYSIRSSSEEIKITVDDKHYQKALNEINQFYQKFPQYLQYPYQGPIVLNPWDQVKRNAGAFTPTVNREQLSTSEENSKKTIDMESTEVFTKKTKLTEEEKNRLNFLKKISQYYQKFAWPQYLKTVDQHQKAMKPWTQPKTNAIPYVRYL

SEQ ID NO: 468 - protein, 223 aa, Ovis aries
MKFFIFTCLLAVALAKHKMEHVSSSEEPINISQEIYKQEKNMAIHPRKEKLCTTSCEEVVRNANEEEYSIRSSSEESAEVAPEEVKITVDDKHYQKALNEINQFYQKFPQYLQYLYQGPIVLNPWDQVKRNAGPFTPTVNREQLSTSEENSKKTIDMESTEVFTKKTKLTEEEKNRLNFLKKISQYYQKFAWPQYLKTVDQHQKAMKPWTQPKTNAIPYVRYL

SEQ ID NO: 469 - protein, 223 aa, Ovis aries
MKFFIFTCLLAVALAKHKMEHVSSSEEPINISQEIYKQEKNMAIHPRKEKLCTTSCEEVVRNADEEEYSIRSSSEESAEVAPEEVKITVDDKHYQKALNEINQFYQKFPQYLQYLYQGPIVLNPWDQVKRNAGPFTPTVNREQLSTSEENSKKTIDMESTEVFTKKTKLTEEEKNRLNFLKKISQYYQKFAWPQYLKTVDQHQKAMKPWTQPKTNAIPYVRYL

SEQ ID NO: 470 - protein, 223 aa, Ovis aries
MKFFIFTCLLAVALAKHKMEHVSSSEEPINISQEIYKQEKNMAIHPRKEKLCTTSCEEVIRNSNEEEYSIRSSSEESAEVAPEEVKITVDDKHYQKALNEINQFYQKFPQYLQYLYQGPIVLNPWDQVKRNAGPFTPTVNREQLSTSEENSKKTIDMESTEVFTKKTKLTEEEKNRLNFLKKISQYYQKFAWPQYLKTVDQHQKAMKPWTQPKTNAIPYVRYL

SEQ ID NO: 471 - protein, 223 aa, Ovis aries
MKFFIFTCLLAVALAKHKMEHVSSSEEPINISQEIYKQEKNMAIHPRKEKLCTTSCEEVVRNANEEEYSIRSSSEESAEVAPEEVKITVDDKHYQKALNEINQFYQKFPQYLQYLYQGPIVLNPWDQVKRNAGPFTPTVNREQLSTSEENSKKTIDMESTEVFTKKTKLTEEEKNHLNFLKKISQYYQKFAWPQYLKTVDQHQKAMKPWTQPKTNAIPYVRYL

SEQ ID NO: 472 - protein, 223 aa, Ovis aries
MKFFIFTCLLAVALAKHKMEHVSSSEEPINISQEIYKQEKNMAIHPRKEKLCTTSCEEVVSNANEEEYSIRSSSEESAEVAPEEVKITVDDKHYQKALNEINQFYQKFPQYLQYLYQGPIVLNPWDQVKRNAGPFTPTVNREQLSTSEENSKKTIDMESTEVFTKKTKLTEEEKNRLNFLKKISQYYQKFAWPQYLKTVDQHQKAMKPWTQPKTNAIPYVRYL

SEQ ID NO: 473 - protein, 223 aa, Ovis aries
MKFFIFTCLLAVALAKHKMEHVSSSEEPINISQEIYKQEKNMAIHPRKEKLCTTSCEEVVRNANEEEYSIRSSSEESAEVAPEEVKITVYDKHYQKALNEINQFYQKFPQYLQYLYQGPVVLNPWDQVKRNAGPFTPTVNREQLSTSEENSKKTIDMESTEVFTKKTKLTEEEKNRLNFLKKISQYYQKFAWPQYLKTVDQHQKAMKPWTQPKTNAIPYVRYL

SEQ ID NO: 474 - protein, 222 aa, Bubalus bubalis
MKFFIFTCLLAVALAKHTMEHVSSSEESIISQETYKQEKNMAIHPSKENLCSTFCKEVIRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEDKNRLNFLKKISQHYQKFAWPQYLKTVYQYQKAMKPWTQPKTNVIPYVRYL

SEQ ID NO: 475 - protein, 222 aa, Bubalus bubalis
MKFFIFTCLLAVALAKHTMEHVSSSEESIISQETYKQEKNMAIHPSKENLCSTFCKEVIRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVITKKTKLTEEDKNRLNFLKKISQHYQKFTWPQYLKTVYQYQKAMKPWTQPKTKVIPYVRYL

SEQ ID NO: 476 - protein, 210 aa, Bubalus bubalis
ALAKHTMEHVSSSEESIISQETYKQEKNMAIHPSKENLCSTFCKEVIRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVITKKTKLTEEDKNRLNFLKKISQHYQKFTWPQYLKTVYQYQKAMKPWTQPKTNVIPYVRYL

SEQ ID NO: 477 - protein, 215 aa, Bubalus bubalis
CLLAVALAKHTMEHVSSSEESIISQETYKQEKNMAIHPSKENLCSTFCKEVIRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVITKKTKLTEEDKNRLNFLKKISQHYQKFTWPQYLKTVYQYQKAMKPWTQPKTNVIPYVRYL

SEQ ID NO: 478 - protein, 222 aa, Bubalus bubalis
MKFFIFTCLLAVALAKHTMEHVSSSEESIISQETYKQEKNMAIHPSKENLCSTFCKEVIRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVITKKTKLTEEDKNRLNFLKKISQHYQKFTWPQYLKTVYQYQKAMKPWTQPKTNVIPYVRYL

SEQ ID NO: 479 - protein, 222 aa, Bubalus bubalis
MKFFIFTCLLAVALAKHTMEHVSSSEESIISQETYKQEKNMAIHPSKENLCSTFCKEVIRNANEEEYSIGSSSEESAEVATEEVKITVDDKHYQKALNEINQFYQKFPQYLQYLYQGPIVLNPWDQVERNAVPITPTLNREQLSTSEENSKKTVDMESTEVITKKTKLTEEDKNRLNFLKKISQHYQKFTWPQYLKTVYQYQKAMKPWTQPKTNVIPYVRYL

SEQ ID NO: 480 - protein, 213 aa, Bubalus bubalis
MKFFIFTCLLAVALAKHTMEHVSSSEESIISQETYKQEKNMAIHPSKENLCSTFCKEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEDKNRLNFLKKISQHYQKFAWPQYLKTVYQYQKAMKPWTQPKTNVIPYVRYL

SEQ ID NO: 481 - protein, 213 aa, Bubalus bubalis
MKFFIFTCLLAVALAKHTMEHVSSSEESIISQETYKQEKNMAIHPSKENLCSTFCKEVIRNANEEEYSIGSSSEESAEVATEEVKITVDDKHYQKALNEINQFYQKFPQYLQYLYQGPIVLNPWDQVKRNAVPITPTLNREQLSTSEESTEVITKKTKLTEEDKNRLNFLKKISQHYQKFTWPQYLKTVYQYQKAMKPWTQPKTNVIPYVRYL

SEQ ID NO: 482 - protein, 213 aa, Bubalus bubalis
MKFFIFTCLLAVALAKHTMEHVSSSEESIISQETYKQEKNMAIHPSKEVIRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVITKKTKLTEEDKNRLNFLKKISQHYQKFTWPQYLKTVYQYQKAMKPWTQPKTNVIPYVRYL

SEQ ID NO: 483 - protein, 222 aa, Bos mutus
MKFFIFTCLLAVALAKNTMEHVSSSEESIISQETYKQEKNMAINPSKGNLCSTFCKEVVRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMKPWIQPKTKVIPYVRYL

SEQ ID NO: 484 - protein, 219 aa, Bos mutus
MKFFIFTCLLAVALAKNTMEHVSSSEESIISQETYKQEKNMAINPSKGNLCSTFCKEVVRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMKPWIQPKTKVIPYV

SEQ ID NO: 485 - protein, 222 aa, Jeotgalicoccus aerolatus
MKFFIFTCLLAVALAKNTMEHVSSSEESIISQETYKQEKNMAINPSKENLCSTFCKEVVRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMKPWIQPKTKVIPYVRYL

SEQ ID NO: 486 - protein, 182 aa, Jeotgalicoccus aerolatus
MAINPSKENLCSTFCKEVVRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMKPWIQPKTKVIPYVRYL

SEQ ID NO: 487 - protein, 237 aa, Jeotgalicoccus coquinae
PGLLVFLLGNEDKVNMKFFIFTCLLAVALAKNTMEHVSSSEESIISQETYKQEKNMAINPSKENLCSTFCKEVVRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMKPWIQPKTKVIPYVRYL

SEQ ID NO: 488 - protein, 222 aa, Bos grunniens
MKFFIFTCLLAVALAKNTMEHVSSSEESIISQETYKQEKNMAINPSKENLCSTFCKEVVRNANEEEYSIGSSSEESAVVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMEPWIQPKTKVIPYVRYL

SEQ ID NO: 489 - protein, 222 aa, Bison bison
MKFFIFTCLLAVALAKNTMEHVSSSEESIISQETYKQEKNMAINPSKENLCSTFCKEVVRNVNEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKFALPQYLKTVYQRQKAMKPWIQPKTKVIPYVRYL

SEQ ID NO: 490 - protein, 414 aa, unidentified
  REGION 1..414: note = Bos indicus x Bos taurus
MKFFIFTCLLAVALAKNTMEHVSSSEESIISQETYKQEKNMAINPSKENLCSTFCKEVVRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMKPWIQPKTKVIPYVDYSDNIGSPYPVNPSASISYPVIPSASIPYPFNPSASISYPVTPSASIPCPVNPSASIPCPMPDRDFTPPVYLTKTNLPNDPAVPSNPLVFTALGAPLYSIAGLPISPFSGQLPPTGVFRLVPVSSRPIGPLYSPNEAAPSANTYIVEILVPPTTPPHTVPDVQPLTITEPDEAEPAADALAAPEPQSSISFDRE

SEQ ID NO: 491 - protein, 222 aa, Odocoileus virginianus
MKFFIFTCLLAVALAKHKMEHVSSSEESINIQETYKQEKNMAIHPSKENLCSTSCKEVVRNANEEEYSISSSSEESAEVATEEVKVTVDDKHYQKILNEISQFYQKFPQYLQYLYQGPIVMNPWEQVKRTAVPFTPTVNREQLSTSEENSEKIVDMESTEVFTKKTKLTEEEKNHLNLLKKISQYYQKFAWPQYLKTVYQYQKAMKPWTQPKTNAIPYVKYL

SEQ ID NO: 492 - protein, 210 aa, Muntiacus muntjak
MKNSAKHHKMEHVSSSEESINIQETYKQEKNMAIHPSKENLCSTSCKEVVRNANEEEYSISSSSEESAEVATEEVKVTVDDKHYQKVLNEISQLYQKFPQYLQYLYQGPIVMNPWEQVKRTAGPFTPTVNRDHLSTSEENSKKIVDMESTEVFTKKTKLTEEEKNRLNFLKKISQYYQKFAWPQYLKTVYQYQKAMKPWTQPKTNAIPYV

SEQ ID NO: 493 - protein, 210 aa, Muntiacus reevesi
MKNSAKHHKMEHVSSSEESINIQETYKQEKNMAIHPSKENLCSTSCKEVVRNANEEEYSISSSSEESAEVATEEAKVTVDDKHYQKVLNEISQLYQKFPQYLQYLYQGPIVMNPWEQVKRTAGPFTPTVNRDHLSTSEENSKKIVDMESTEVFTKKTKLTEEEKNRLNFLKKISQYYQKFAWPQYLKTVYQYQKAMKPWTQPKTNAIPYV

SEQ ID NO: 494 - protein, 235 aa, Cervus elaphus
MKFFIFTCLLAVALAKHKMEHVSSSEESINIQETYKQEKNMAIHPSKEVVRNANEEESADVATEEVKVTVDNKHYQKVLNEISQFYQKFPQYLQYLYQGPIVMNPWEQVKRTAGPFIPTVNRDHLSTSEENSKKIVDMESTEVFTKKTKLTEEEKNRLNFLKKISQYYQKFAWPQYLKTVYQYQKAMKPWTQPKTNAIPYVVELENLSSTVSYLPLSTTSMFGVAHQQDKSQSIS

SEQ ID NO: 495 - protein, 224 aa, Bos taurus
MKVLILACLVALALARELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKQKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 496 - protein, 224 aa, Bos taurus
MKVLILACLVALALARELEELNVPGEIVESLSSSEESITRINKKIEKFQSEEQQQTEDELQDKIHPFAQTQSLVYPFPGPIHNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 497 - protein, 209 aa, Bos taurus
RELEELNVPGEIVESLSSSEESITRINKKIEKFQSEEQQQTEDELQDKIHPFAQTQSLVYPFPGPIHNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 498 - protein, 224 aa, Bos taurus
MKVLILACLVALALARELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKGAMAPKHKEMPFPKYPVEPLTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 499 - protein, 224 aa, Bos taurus
MKVLILACLVALALARELEELNVPGEIVESLSSSEESITRINKKIEKFQSEEQQQTEDELQDKIHPFAQTQSLVYPFPGPIHNSLPQNIPPLTQTPVVVPPFLQPEVMGVSKVKEAMAPKHKEMPFPKYPVEPFTERQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 500 - protein, 259 aa, Bos taurus
MPLNTIYKQPQNQIIIHSAPPSLLVLYFGKKELRAMKVLILACLVALALARELEELNVPGEIVESLSSSEESITRINKKIEKFQSEEQQQTEDELQDKIHPFAQTQSLVYPFPGPIHNSLPQNIPPLTQTPVVVPPFLQPEVMGVSKVKEAMAPKHKEMPFPKYPVEPFTERQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 501 - protein, 224 aa, Bos taurus
MKVLILACLVALALARELEELNVPGEIVESLSSSEESITRINKKIEKFQSEEQQQTEDELQDKIHPFAQTQSLVYPFPGPIPNSLPQNIPPLTQTPVVVPPFLQPEVLGVSKVKEAMAPKHKEMPFPKYPVEPFTESQSLTLTDVENLHLPPLLLQSWMHQPHQPLPPTVMFPPQSVLSLSQSKVLPVPQKAVPYPQRDMPIQAFLLYQQPVLGPVRGPFPIIV

SEQ ID NO: 502 - protein, 216 aa, Bos taurus
MKVLILACLVALALARELEELNVPGEIVESLSSSEKIEKFQSEEQQQTEDELQDKIHPFAQTQSLVYPFPGPIHNSLPQNIPPLTQTPVVVPPFLQPEVMGVSKVKEAMAPKHKEMPFPKYPVEPFTERQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 503 - protein, 166 aa, Bos taurus
DELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPALGPVRGPFPII

SEQ ID NO: 504 - protein, 165 aa, Bos taurus
DELQDKIPPLPEQSLVYPFPGPIHNSLPQNIPPLTQTPVVVPPFLQPEVMGVSRVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPII

SEQ ID NO: 505 - protein, 149 aa, Bos taurus
DELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFL

SEQ ID NO: 506 - protein, 149 aa, Bos taurus
DELQDKIHPFAQTQSLVYPFPGPIHNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFL

SEQ ID NO: 507 - protein, 146 aa, Bos taurus
KIHPFAQTQSLVYPFPGPIHNSLPQNIPPLTQTPVVVPPFLQPEVMGVSKVKEAMAPKHKEMPFPKYPVEPFTERQSLTLTDVENLHLPLPLLQSWMHQPHQPLPPTVMFPPQSVLSLSQSKVLPVPQKAVPYPQRDMPIQAFLLV

SEQ ID NO: 508 - protein, 222 aa, Capra hircus
MKVLILACLVALAIAREQEELNVVGETVESLSSSEESITHINKKIEKFQSEEQQQTEDELQDKIHPFAQAQSLVYPFTGPIPNSLPQNILPLTQTPVVVPPFLQPEIMGVPKVKETMVPKHKEMPFPKYPVEPFTESQSLTLTDVEKLHLPLPLVQSWMHQPPQPLSPTVMFPPQSVLSLSQPKVLPVPQKAVPQRDMPIQAFLLYQEPVLGPVRGPFPILV

SEQ ID NO: 509 - protein, 222 aa, Capra hircus
MKVLILACLVALAIAREQEELNVVGETVESLSSSEESITHINKKIEKFQSEEQQQTEDELQDKIHPFAQAQSLVYPFTGPIPNSLPQNILPLTQTPVVVPPFLQPEIMGVPKVKETMVPKHKEMPFPKYPVEPFTESQSLTLTDVEKLHLPLPLVQSWMHQPPQPLSPTVMFPPQSVLSLSQPKVLPVPQKVVPQRDMPIQAFLLYQEPVLGPVRGPFPILV

SEQ ID NO: 510 - protein, 222 aa, Capra hircus
MKVLILACLVALALAREQEELNVVGETVESLSSSEESITHINKKIEKFQSEEQQQTEDELQDKIHPFAQAQSLVYPFTGPIPNSLPQNILPLTQTPVVVPPFLQPEIMGVPKVKETMVPKHKEMPFPKYPVEPFTESQSLTLTDVEKLHLPLPLVQSWMHQPPQPLSPTVMFPPQSVLSLSQPKVLPVPQKAVPQRDMPIQAFLLYQEPVLGPVRGPFPILV

SEQ ID NO: 511 - protein, 223 aa, Capra hircus
MKVLILACLVALAIAREQEELNVVGETVESLSSSEESITHINKKIEKFQSEEQQQTEDELQDKIHPFAQAQSLVYPFTGPIPNSLPQNILPLTQTPVVVPPFLQPEIMGVPKVKETMVPKHKEMPFPKYPVEPFTESQSLTLTDVEKLHLPLPLVQSWMHQPPQPLSPTVMFPPQSVLSLSQPKVLPVPQKVVPQRDMPIQAFLLYQEPVLGPVRGPFPILVV

SEQ ID NO: 512 - protein, 257 aa, Capra hircus
MPLNTIYKQPLNQIIIHSASPSLLLLYFGKKESRAMKVLILACLVALAIAREQEELNVVGETVESLSSSEESITHINKKIEKFQSEEQQQTEDELQDKIHPFAQAQSLVYPFTGPIPNSLPQNILPLTQTPVVVPPFLQPEIMGVPKVKETMVPKHKEMPFPKYPVEPFTESQSLTLTDVEKLHLPLPLVQSWMHQPPQPLSPTVMFPPQSVLSLSQPKVLPVPQKVVPQRDMPIQAFLLYQEPVLGPVRGPFPILV

SEQ ID NO: 513 - protein, 222 aa, Capra hircus
MKVLILACLVALAIAREQEELNVVGETVESLSSSEESITHINKKIEKFQSEEQQQTEDELQDKIHPFAQAQSLVYPFTGPIPNSLPQNILPLTQTPVVVPPFLQPEIMGVPKVKETMVPKHKEMPFPKYPVEPFTESQSLTLTDVEKLHLPLPLVQSWMHQPPQPLSPTVMFPPQSVLSLSQPKVLPVPQKVVPQRDMPIQAFLLYQEPVLGPVRGPFPILN

SEQ ID NO: 514 - protein, 257 aa, Capra hircus
MPLNTIYKQPLNQIIIHSASPSLLLLYFGKKESRAMKVLILACLVALAIAREQEELNVVGETVESLSSSEESITHINKKIEKFQSEEQQQTEDELQDKIHPFAQAQSLVYPFTGPIPNSLPQNILPLTQTPVVVPPFLQPEIMGVPKVKETMVPKHKEMPFPKYPVEPFTESQSLTLTDVEKLHLPLPLVQSWMHQPPQPLSPTVMFPPQSVLSLSQPKVLPVPQKVVPQRDMPIQAFLLYQEPVLGPVRGPFPILN

SEQ ID NO: 515 - protein, 222 aa, Capra hircus
MKVLILACLVALDIAREQEELNVVGETVESLSSSEESITHINKKIEKFQSEEQQQTEDELQDKIHPFAQAQSLVYPFTGPIPNSLPQNILPLTQTPVVVPPFLQPEIMGVPKVKETMVPKHKEMPFPKYPVEPFTESQSLTLTDVEKLHLPLPLVQSWMHQPPQPLSPTVMFPPQSVLSLSQPKVLPVPQKVVPQRDMPIQAFLLYQEPVLGPVRGPFPILN

SEQ ID NO: 516 - protein, 160 aa, Capra hircus
DELQDKIHPFAQAQSLVYPFTGPIPNSLPQNILPLTQTPVVVPPFLQPEIMGVPKVKETMVPKHKEMPFPKYPVEPFTESQSLTLTDVEKLHLPLPLVQSWMHQPPQPLSPTVMFPPQSVLSLSQPKVLPVPQKAVPQRDMPIQAFLLYQEPVLGPVRGP

SEQ ID NO: 517 - protein, 188 aa, Capra hircus
MKVLILACLVALALAREQEELNVVGETVESLSSSEESITHINKKIEKFQSEEQQQTEDELQDKIHPFAQAQSLVYPFTGPIPNSLPQNILPLTQTPVVVPPFLQPEIMGVPKVKETMVPKHKEMPFPKYPVEPFTESQSLTLTDVEKLHLPLPLVQSWMHQPPQPLSPTVMFPPQSVLSLSQPKVLPV

SEQ ID NO: 518 - protein, 160 aa, Capra hircus
DELQDKIHPFAQAQSLVYPFTGPIPNSLPQNILPLTQTPVVVPPFLQPEIMGVPKVKETMVPKHKEMPFPKYPVEPFTESQSLTLTDVEKLHLPLPLVQSWMHQPPQPLSPTVMFPPQSVLSLSQPKVLPVPQKVVPQRDMPIQAFLLYQEPVLGPVRGP

SEQ ID NO: 519 - protein, 181 aa, Capra hircus
MKVLILACLVALAIAREQEELNVVGETVESLSSSEESITHINKKIEKFQSEEQQQTEDELQDKIHPFAQAQSLVYPFTGPIPNSLPQNILPLTQTPVVVPPFLQPEIMGVPKVKETMVPKHKEMPFPKYPVEPFTESQSLTLTDVEKLHLPLPLVQSWMHQPPQPLSPTVMFPPQSVLSLS

SEQ ID NO: 520 - protein, 149 aa, Capra hircus
QAQSLVYPFTGPIPNSLPQNILPLTQTPVVVPPFLQPEIMGVPKVKETMVPKHKEMPFPKYPVEPFTESQSLTLTDVEKLHLPLPLVQSWMHQPPQPLSPTVMFPPQSVLSLSQPKVLPVPQKAVPQRDMPIQAFLLYQEPVLGPVRGP

SEQ ID NO: 521 - protein, 148 aa, Capra hircus
DEVQDKIHPFAQAQSLVYPFTGPIPNSLPQNILPLTQTPVVVPPFLQPEIMGVPKVKETMVPKHKEMPFPKYPVEPFTESQSLTLTDVEKLHLPLPLVQSWMHQPPQPLSPTVMFPPQSVLSLSQPKVLPVPQKAVPQRDMPIQAFLL

SEQ ID NO: 522 - protein, 222 aa, Ovis aries
MKVLILACLVALALAREQEELNVVGETVESLSSSEESITHINKKIEKFQSEEQQQTEDELQDKIHPFAQAQSLVYPFTGPIPNSLPQNILPLTQTPVVVPPFLQPEIMGVPKVKETMVPKHKEMPFPKYPVEPFTESQSLTLTDVEKLHLPLPLVQSWMHQPPQPLPPTVMFPPQSVLSLSQPKVLPVPQKAVPQRDMPIQAFLLYQEPVLGPVRGPFPILV

SEQ ID NO: 523 - protein, 222 aa, Ovis aries
  VARIANT 7: note = X is any amino acid
MKVLILXCLVALALAREQEELNVVGETVESLSSSEESITHINKKIEKFQSEEQQQTEDELQDKIHPFAQAQSLVYPFTGPIPNSLPQNILPLTQTPVVVPPFLQPEIMGVPKVKETMVPKHKEMPFPKYPVEPFTESQSLTLTDVEKLHLPLPLVQSWMHQPPQPLPPTVMFPPQSVLSLSQPKVLPVPQKAVPQRDMPIQAFLLYQEPVLGPVRGPFPILV

SEQ ID NO: 524 - protein, 224 aa, Bubalus bubalis
MKVLILACLVALALARELEELNVPGEIVESLSSSEESITHINKKIEKFQSEEQQQTEDELQDKIHPFAQTQSLVYPFPGPIPNSLPQNIPPLTQTPVVVPPFLQPEIMGVSKVKEAMAPKHKEMPFPKYPVEPFTESQSLTLTDVENLHLPLPLLQSWMHQPPQPLPPTVMFPPQSVLSLSQSKVLPVPQKAVPYPQRDMPIQAFLLYQEPVLGPVRGPFPIIV

SEQ ID NO: 525 - protein, 259 aa, Bubalus bubalis
MPLNTIYKQPQNQIIIHSAPPSLLVLYFGKKKLRVMKVLILACLVALALARELEELNVPGEIVESLSSSEESITHINKKIEKFQSEEQQQTEDELQDKIHPFAQTQSLVYPFPGPIPNSLPQNIPPLTQTPVVVPPFLQPEIMGVSKVKEAMAPKHKEMPFPKYPVEPFTESQSLTLTDVENLHLPLPLLQSWMHQPPQPLPPTVMFPPQSVLSLSQSKVLPVPQKAVPYPQRDMPIQAFLLYQEPVLGPVRGPFPIIV

SEQ ID NO: 526 - protein, 224 aa, Bubalus bubalis
MKVLILACLVALALARELEELNVPGEIVESLSSSEESITHINKKIEKFQSEEQQQTEDELQDKIHPFAQTQSLVYPFPGPIPNSLPQNIPPLTQTPVVVPPFLQPEIMGVSKVKEAMAPKHKEMPFPKYPFEPFTESQSLTLTDVENLHLPLPLLQSWMHQPPQPLPPTVMFPPQSVLSLSQSKVLPVPQKAVPYPQRDMPIQAFLLYQEPVLGPVRGPFPIIV

SEQ ID NO: 527 - protein, 224 aa, Bubalus bubalis
MKVLILACLVALALARELEELNVPSEIVESLSSSEESITHINKKIEKFQSEEQQQTEDELQDKIHPFAQTQSLVYPFPGPIPNSLPQNIPPLTQTPVVVPPFLQPEIMGVSKVKEAMAPKHKEMPFPKYPVEPFTESQSLTLTDVENLHLPLPLLQSWMHQPPQPLPPTVMFPPQSVLSLSQSKVLPVPQKAVPYPQRDMPIQAFLLYQEPVLGPVRGPFPIIV

SEQ ID NO: 528 - protein, 224 aa, Bubalus bubalis
MKVLILACLVALALARELEELNVPSEIVESLSSSEESITHINKKIEKFQSEEQRQTEDELQDKIHPFAQTQSLVYPFPGPIPNSLPQNIPPLTQTPVVVPPFLQPEIMGVSKVKEAMAPKHKEMPFPKYPVEPFTESQSLTLTDVENLHLPLPLLQSWMHQPPQPLPPTVMFPPQSVLSLSQSKVLPVPQKAVPYPQRDMPIQAFLLYQEPVLGPVRGPFPIIV

SEQ ID NO: 529 - protein, 224 aa, Bubalus bubalis
MKVLILACLVALALARELEELNVPGEIVESLSSSEESITHINKKIEKFQSEEQQQMEDELQDKIHPFAQTQSLVYPFPGPIPKSLPQNIPPLTQTPVVVPPFLQPEIMGVSKVKEAMAPKHKEMPFPKYPVEPFTESQSLTLTDVENLHLPLPLLQSWMHQPPQPLPPTVMFPPQSVLSLSQSKVLPVPQKAVPYPQRDMPIQAFLLYQEPVLGPVRGPFPIIV

SEQ ID NO: 530 - protein, 259 aa, Bubalus bubalis
MPLNTIYKQPQNQIIIHSAPPSLLVLYFGKKKLRAMKVLILACLVALALARELEELNVPGEIVESLSSSEESITHINKKIEKFQSEEQQQMEDELQDKIHPFAQTQSLVYPFPGPIPKSLPQNIPPLTQTPVVVPPFLQPEIMGVSKVKEAMAPKHKEMPFPKYPVEPFTESQSLTLTDVENLHLPLPLLQSWMHQPPQPLPPTVMFPPQSVLSLSQSKVLPVPQKAVPYPQRDMPIQAFLLYQEPVLGPVRGPFPIIV

SEQ ID NO: 531 - protein, 224 aa, Bubalus bubalis
MKVLILACLVALALARELEELNVPGEIVESLSSSEESITHINKKIEKFQSEEQQQMEDELQDKIHPFAQTQSLVYPFPGPIPKSLPQNIPPLTQTPVVVPPFLQPEIMGVSKVKEATAPKHKEMPFPKYPVEPFTESQSLTLTDVENLHLPLPLLQSWMHQPPQPLPPTVMFPPQSVLSLSQSKVLPVPQKAVPYPQRDMPIQAFLLYQEPVLGPVRGPFPIIV

SEQ ID NO: 532 - protein, 209 aa, Bubalus bubalis
RELEELNVPGEIVESLSSSEESITHINKKIEKFQSEEQQQTEDELQDKIHPFAQTQSLVYPFPGPIPKSLPQNIPPLTQTPVVVPPFLQPEIMGVSKVKEAMAPKHKEMPFPKYPVQPETESQSLTLTDVENLHLPPLLLQSWMHQPHQPLPPTVMFPPQSVLSLSQSKVLPVPEKAVPYPQRDMPIQAFLLYQEPVLGPVRGPFPIIV

SEQ ID NO: 533 - protein, 166 aa, Bubalus bubalis
DELQDKIHPFAQTQSLVYPFPGPIPNSLPQNIPPLTQTPVVVPPFLQPEIMGVSKVKEAMAPKHKEMPFPKYPVEPFTESQSLTLTDVENLHLPLPLLQSWMHQPPQPLPPTVMFPPQSVLSLSQSKVLPVPQKAVPYPQRDMPIQAFLLYQEPVLGPVRGPFPII

SEQ ID NO: 534 - protein, 166 aa, Bubalus bubalis
DELQDKIHPFAQTQSLVYPFPGPIPKSLPQNIPPLTQTPVVVPPFLQPEIMGVSKVKEAMAPKHKEMPFPKYPVEPFTESQSLTLTDVENLHLPLPLLQSWMHQPPQPLPPTVMFPPQSVLSLSQSKVLPVPQKAVPYPQRDMPIQAFLLYQEPVLGPVRGPFPII

SEQ ID NO: 535 - protein, 166 aa, Bubalus bubalis
DELQDKNHPFAQTQSLVYPFPGPIPKSLPQNIPPLTQTPVVVPPFLQPEIMGVSKVKEAMAPKHKEMPFPKYPVEPFTESQSLTLTDVENLHLPLPLLQSWMHQPPQPLPPTVMFPPQSVLSLSQSKVLPVPQKAVPYPQRDMPIQAFLLYQEPVLGPVRGPFPII

SEQ ID NO: 536 - protein, 165 aa, Bubalus bubalis
DELQDKIPPLPEQSLVYPFPAPIPKSLPQNIPPLTQTPVVVPPFLQPEIMGVSKVKEAMAPKHKEMPFPKYPVEPFTESQSLTLTDVENLHLPLPLLQSWMHQPPQPLPPTVMFPPQSVLSLSQSKVLPVPQKAVPYPQRDMPIQAFLLYQEPVLGPVRGPFPII

SEQ ID NO: 537 - protein, 155 aa, Bubalus bubalis
IHPFAQTQSLVYPFPGPIPNSLPQNIPPLTQTPVVVPPFLQPEIMGVSKVKEAMAPKHKEMPFPKYPVEPFTESQSLTLTDVENLHLPLPLLQSWMHQPPQPLPPTVMFPPQSVLSLSQSKVLPVPQKAVPYPQRDMPIQAFLLYQEPVLGPVRG

SEQ ID NO: 538 - protein, 155 aa, Bubalus bubalis
IHPFAQTQSLVYPFPGPIPKSLPQNIPPLTQTPVVVPPFLQPEIMGVSKVKEAMAPKHKEMPFPKYPVEPFTESQSLTLTDVENLHLPLPLLQSWMHQPPQPLPPTVMFPPQSVLSLSQSKVLPVPQKAVPYPQRDMPIQAFLLYQEPVLGPVRG

SEQ ID NO: 539 - protein, 235 aa, Bos mutus
MKVLILACLVALALARELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIVSLNLLTVLFNF

SEQ ID NO: 540 - protein, 270 aa, Bos mutus
MPLNTIYKQPQNQIIIHSAPPSLLVLYFGKKELRAMKVLILACLVALALARELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIVSLNLLTVLFNF

SEQ ID NO: 541 - protein, 259 aa, Bos mutus
MPLNTIYKQPQNQIIIHSAPPSLLVLYFGKKELRAMKVLILACLVALALARELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 542 - protein, 216 aa, Bos mutus
MKVLILACLVALALARELEELNVPGEIVESLSSSEKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 543 - protein, 227 aa, Bos mutus
MKVLILACLVALALARELEELNVPGEIVESLSSSEKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIVSLNLLTVLFNF

SEQ ID NO: 544 - protein, 224 aa, Bos indicus
  VARIANT 197: note = X is any amino acid
MKVLILACLVALALARELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPXRDMPIQAFLLYQEPVLGPVRGPFPIIV

SEQ ID NO: 545 - protein, 224 aa, Bos indicus
  VARIANT 197: note = X is any amino acid
MKVLILACLVALALARELEELNVPGEIVESLSSSEESITRINKKIEKFQSEEQQQTEDELQDKIHPFAQTQSLVYPFPGPIHNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPXRDMPIQAFLLYQEPVLGPVRGPFPIIV

SEQ ID NO: 546 - protein, 165 aa, Bos indicus
DELQDKIPPLPEQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPII

SEQ ID NO: 547 - protein, 155 aa, Bos indicus
IHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRG

SEQ ID NO: 548 - protein, 152 aa, Bos indicus
QSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPI

SEQ ID NO: 549 - protein, 154 aa, Bos indicus
LPDQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFP

SEQ ID NO: 550 - protein, 224 aa, Bison bison
MKVLILACLVALALARELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 551 - protein, 177 aa, Jeotgalicoccus aerolatus
FQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 552 - protein, 240 aa, Jeotgalicoccus coquinae
PPSLLVLYFGKKELRAMKVLILACLVALALARELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 553 - protein, 224 aa, unidentified
  REGION 1..224: note = Bos indicus x Bos taurus
MKVLILACLVALALARELEELNVPGEIVESLSSSEESITRINKKIEKFQSEEQQQTEDELQEKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 554 - protein, 153 aa, unidentified
  REGION 1..153: note = Bos indicus x Bos taurus
QSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIL

SEQ ID NO: 555 - protein, 150 aa, unidentified
  REGION 1..150: note = Bos indicus x Bos taurus
DEVQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLL

SEQ ID NO: 556 - protein, 259 aa, Bos grunniens
MPLNTIYKQPQNQIIIHSAPPSLLVLYFGKKELGAMKVLILACLVALALARELEELNVPGEIVEGLSSSEESITRINKKIEKFQSEEQQQTEDGLQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 557 - protein, 166 aa, Bos grunniens
DELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPII

SEQ ID NO: 558 - protein, 166 aa, Bos grunniens
DELQDKIHPFAQTQSLVYPFPGPIPNSLPQNIPPLTQTPVVVPPFLQPEVMGVSKVKEAMAPKHEEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPII

SEQ ID NO: 559 - protein, 155 aa, Bos grunniens
QSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIFVS

SEQ ID NO: 560 - protein, 224 aa, Neophocaena asiaeorientalis
MKVLILACLLALALAREKEELIVSGETVKSLSSSEESVTHINEKIEKFKHEEQQQTEDERQDRIHHFSQPQPLVYSYTGPIPYPILPKNILPLAQPPVVVPFPQPEIMEVPKAKETILPKHKEMRFPKSPVEPFIESQSLTLTDLENLHLPLPLLQSLMHQPPHRLPPTLMFPPQPLQSLSQSKVLPIPQQVVPYLQRDMPIQTLLLYQEPVLGPIQGLYPVIV

SEQ ID NO: 561 - protein, 258 aa, Odocoileus virginianus
MPLNTIYKQPQNQIIIHSGSPSLLVLYVGKEELRAMKVLILACLVALALAKEEELNVSSEIVESISSSEESITHITKKIEKLQSEGQQQAEDELQDKIHPFAQTQSLVYPFTGPIPYSLPQNFLPLPQTPVMVPPFLQPEIMGVSEVKETMVPKHKEMPFPKYPVEPFAEGQSLTLTEAENLHFPLPLPQSWMHQTPQPLPPAVMFPPQSVLSLSQPKVLSVPQKAVPYPQRDMPIQAFLLYQEPVPGPVRGPFPFIV

SEQ ID NO: 562 - protein, 222 aa, Muntiacus reevesi
MKVLILACLVALALAREEELNVSGERLSSSSKAESITHITKKIEKLQSEGQQQTEAELQDKIHPFAQTQPLVYPFTGPIPYSLPQNFLPLPQTPMVVPPFLQPEIMGVSEVKETMVPKHKEMPFPKYPVEPFAEGQSLTLTDVENLHLPLPVLQPWMHQTPQPLPPTVMFPPQSVLSLSQPKVLSVPQKAVPYPQREMPIQAFLLYQEPVPGPVPGPFPIIV

SEQ ID NO: 563 - protein, 230 aa, Muntiacus muntjak
MKVLILACLVALALAREEELNVSGESRTLLKRLSSSSSSKAESITHITKKIEKLPSEGQQQTEAELQDKIHPFAQTQPLVYPFTGPIPYSLPQNFLPLPQTPMVVPPFLQPEIMGVSEVKETMVPKHKEMPFPKYPVEPFAEGQSLTLTDVENLHLPLPVLQPWMHQTPQPLPPTVMFPPQSVLSLSQPKVLSVPQKAVPYPQREMPIQAFLLYQEPVPGPVPGPFPIIV

SEQ ID NO: 564 - protein, 163 aa, Bos taurus
MLIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHI

SEQ ID NO: 565 - protein, 162 aa, Bos taurus; identical to SEQ ID NO: 10

SEQ ID NO: 566 - protein, 178 aa, Bos taurus
MKCLLLALALTCGAQALIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHI

SEQ ID NO: 567 - protein, 161 aa, Bos taurus
IVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHI

SEQ ID NO: 568 - protein, 162 aa, Bos taurus
LIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLVCQCLVRTPEVDDEALEKFDKALKALPMHIRLSFNPTQLEEQCHI

SEQ ID NO: 569 - protein, 162 aa, Bos taurus
LIVTQTMKGLDIQKVAGTWYSLAMAASDISLLDAQSAPLRVYVEELKPTPEGDLEFLLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHI

SEQ ID NO: 570 - protein, 162 aa, Bos taurus
LIVTQTMKGLDIQKVAGTWYSLAMAASDISLLDAQSAPYRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHI

SEQ ID NO: 571 - protein, 178 aa, Bos taurus
MKCLLLALALTCGAQALIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLVCQCLVRTPEVDDEALEKFDKALKALPMHIRLSFNPTQLEEQCHI

SEQ ID NO: 572 - protein, 162 aa, Bos taurus
ASVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHI

SEQ ID NO: 573 - protein, 162 aa, Bos taurus
ASVTQTMKGLDIQKVAGTWYSLAMAASDISLLDAQSAPLRVYVEELKPTPEGDLEILFQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHI

SEQ ID NO: 574 - protein, 162 aa, Bos taurus
ASVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCLENSAEPEQSLACQCLVRTPEVDDEALEKFDKALKALPMHIRLSFNPTQLEEQCHI

SEQ ID NO: 575 - protein, 162 aa, Bos taurus
LIVTQTMKGLDIQKVAGTWYSLAMAASDISLLDAQSAPLRVYVEELKPTPEGDLEILLQKWENDECAQKKIIAEKTKIPAVFKIDALNENKVLVLDTDYKKYLLFCMENSAEPEQSLVCQCLVRTPEVDDEALEKFDKALKALPMHIRLSFNPTQLEEQCHI

SEQ ID NO: 576 - protein, 162 aa, Bos taurus
ASVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLLCMENSAEPEQSLACQCLVRTPEVDDEALEKFDKALKALPMHIRLSFNPTQLEEQCHI

SEQ ID NO: 577 - protein, 178 aa, Bos taurus
MKCLLLALALTCGAQALIVTQTMKGLDIQKVAGTWYSLAMAASDISLLDAQSAPLRVYVEELKPTPEGDLEILLQKWENDECAQKKIIAEKTKIPAVFKIDALNENKVLVLDTDYKKYLLFCMENSAEPEQSLVCQCLVRTPEVDDEALEKFDKALKALPMHIRLSFNPTQLEEQCHI

SEQ ID NO: 578 - protein, 162 aa, Bos taurus
ASVTQTMKGLDIQKVAGTWYSLAMAASDISLLDAQSAPARVYVEELKPTPEGDLEFLLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHI

SEQ ID NO: 579 - protein, 162 aa, Bos taurus
ASVTQTMKGLDIQKVAGTWYSLAMAASDISLLDAQSAPARVYVEELKPTPEGDLEILLQKWENGECAQKKIIAEKTKIPAVFKIDALNENKVLVLDTDYKKYLLLCMENSAEPEQSLACQCLVRTPEVDDEALEKFDKALKALPMHIRLSFNPTQLEEQCHI

SEQ ID NO: 580 - protein, 162 aa, Bos taurus
LIVTQTMKGLDIQKVAGTWYSLAMAASDISLLDAQSAPLRVYVEELKPTPEGDLEILLQKWENDECAQKKIIAEKTKIPAVFKIDALNENKVLVLDTDYKKYLLFCMENSAEPEQSLVCQSLVRTPEVDDEALEKFDKALKALPMHIRLSFNPTQLEEQCHI

SEQ ID NO: 581 - protein, 178 aa, Bos taurus
MKCLLLALALTCGAQALIVTQTMKGLDIQKVAGTWYSLAMAASDISLLDAQSAPLRVYVEELKPTPEGDLEILLQKWENDECAQKKIIAEKTKIPAVFKIDALNENKVLVLDTDYKKYLLVCMENSAEPEQSLVCQCLVRTPEVDDEALEKFDKALKALPMHIRLSFNPTQLEEQCHI

SEQ ID NO: 582 - protein, 163 aa, Bos taurus
AAYVTQTMKGLDIQKVAGTWYSLAMAASDISLLDAQSAPLRVYVEELKPTPEGDLEILLQKWENDECAQKKIIAEKTKIPAVFKIDALNENKVLVLDTDYKKYLLFCMENSAEPEQSLVCQCLVRTPEVDDEALEKFDKALKALPMHIRLSFNPTQLEEQCHI

SEQ ID NO: 583 - protein, 162 aa, Bos taurus
ASVTQTMKGLDIQKVAGTWYSLAMAASDISLLDAQSAPARVYVEELKPTPEGDLEFLLQKWENGECAQKKIIAEKTKIPAVFKIDALNENKVLVLDTDYKKYLLFCFENSAEPEQSLACQCLVRTPEVDDEALEKFDKALKALPMHIRLSFNPTQLEEQCHI

SEQ ID NO: 584 - protein, 162 aa, Bos taurus
ASVTQTMKGLDIQKVAGTWYSLAMAASDISLLDAQSAPARVYVEELKPTPEGDLEILLQKWENGECAQKKIIAEKTKIPAVFKIDALNENKVLVLDTDYKKYLLLCFENSAEPEQSLACQCLVRTPEVDDEALEKFDKALKALPMHIRLSFNPTQLEEQCHI

SEQ ID NO: 585 - protein, 162 aa, Bos taurus
LIVTQTMKGLDIQKVAGTWYSLAMAASDISLLDAQSAPLRVYVEELKPTPEGDLEILLQKWENDECAQKKIIAEKTKIPAVFKLDAINENKVLVLDTDYKKYLLFCMENSAEPEQSLVCQCLVRTPEVDDEALEKFDKALKALPMHIRLSFNPTLQEEQCHI

SEQ ID NO: 586 - protein, 252 aa, Bos taurus
MKCLLLALALTCGAQALIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEGEHPGPTLLLGQEATRPRTTSSHGDPQLPRPPGRMETGCRAPRWPPPHPLPSSLCPGVSSPILTLPRHGSPSPTEQCHI

SEQ ID NO: 587 - protein, 151 aa, Bos taurus
IQKVAGTWYSLAMAASDISLLDAQSAPLRVYVEELKPTPEGDLEILLQKWENDECAQKKIIAEKTKIPAVFKIDALNENKVLVLDTDYKKYLLFCMENSAEPEQSLVCQCLVRTPEVDDEALEKFDKALKALPMHIRLSFNPTQLEEQCHI

SEQ ID NO: 588 - protein, 162 aa, Capra hircus
IIVTQTMKGLDIQKVAGTWYSLAMAASDISLLDAQSAPLRVYVEELKPTPEGNLEILLQKWENGECAQKKIIAEKTKIPAVFKIDALNENKVLVLDTDYKKYLLFCMENSAEPEQSLACQCLVRTPEVDKEALEKFDKALKALPMHIRLAFNPTQLEGQCHV

SEQ ID NO: 589 - protein, 180 aa, Capra hircus
MKCLLLALGLALACGIQAIIVTQTMKGLDIQKVAGTWYSLAMAASDISLLDAQSAPLRVYVEELKPTPEGNLEILLQKWENGECAQKKIIAEKTKIPAVFKIDALNENKVLVLDTDYKKYLLFCMENSAEPEQSLACQCLVRTPEVDKEALEKFDKALKALPMHIRLAFNPTQLEGQCHV

SEQ ID NO: 590 - protein, 169 aa, Capra hircus
MKCLLLALGLALACGIQAIIVTQTMKGLDIQKVAGTWYSLAMAASDISLLDAQSAPLRVYVEELKPTPEGNLEILLQKWENGECAQKKIIAEKTKIPAVFKIDALNENKVLVLDTDYKKYLLFCMENSAEPEQSLACQCLVRTPEVDKEALEKFDKALKALPMHIRLAF

SEQ ID NO: 591 - protein, 162 aa, Ovis aries
IIVTQTMKGLDIQKVAGTWYSLAMAASDISLLDAQSAPLRVYVEELKPTPEGNLEILLQKWENGECAQKKIIAEKTKIPAVFKIDALNENKVLVLDTDYKKYLLFCMENSAEPEQSLACQCLVRTPEVDNEALEKFDKALKALPMHIRLAFNPTQLEGQCHV

SEQ ID NO: 592 - protein, 180 aa, Ovis aries
MKCLLLALGLALACGVQAIIVTQTMKGLDIQKVAGTWHSLAMAASDISLLDAQSAPLRVYVEELKPTPEGNLEILLQKWENGECAQKKIIAEKTKIPAVFKIDALNENKVLVLDTDYKKYLLFCMENSAEPEQSLACQCLVRTPEVDNEALEKFDKALKALPMHIRLAFNPTQLEGQCHV

SEQ ID NO: 593 - protein, 161 aa, Ovis aries
VTQTMKGLDIQKVAGTWYHSLAMAASDISLLDAQSAPLRVYVEELKPTPEGNLEILLQKWENGECAQKKIIAEKTKIPAVFKIDALNENKVLVLDTDYKKYLLFCMENSAEPEQSLACQCLVRTPEVDNEALEKFDKALKALPMHIRLAFNPTQLEGQCHV

SEQ ID NO: 594 - protein, 162 aa, Ovis aries
IIVTQTMKGLDIQKVAGTWYHSLAMAASDISLLDAQSAPLRVYVEELKPTPEGNLEILLQKWEGECAQKKIIAEKTKIPAVFKIDALNENKVLVLDTDYKKYLLFCMENSAEPEQSLACQCLVRTPEVDNEALEKFDKALKALPMHIRLAFNPTQLEGQCHV

SEQ ID NO: 595 - protein, 162 aa, Bubalus bubalis
IIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHV

SEQ ID NO: 596 - protein, 180 aa, Bubalus bubalis
MKCLLLALGLALACAAQAIIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHV

SEQ ID NO: 597 - protein, 180 aa, Bubalus bubalis
MKCLLLALGLALACGAQAIIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHV

SEQ ID NO: 598 - protein, 180 aa, Bubalus bubalis
MKCLLLALGLALACGTQAIIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHV

SEQ ID NO: 599 - protein, 180 aa, Bubalus bubalis
MKCLLLALGLALACGTQAIIVTQTMKGLDIQKVAGTWYSLAMAVSDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHV

SEQ ID NO: 600 - protein, 178 aa, Bubalus bubalis
MKCLLLALALACGAQAIIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEKAWPASAWVRTPEVDDEALEKFDKALKALPMHIRLSFNPTQLEEQCHI

SEQ ID NO: 601 - protein, 178 aa, Bos mutus
MKCLLLALALTCGAQALIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEGQCHI

SEQ ID NO: 602 - protein, 298 aa, Bos mutus
MQLKVPPQVAGMWHTVAMAASNMLLLDAESGPLRVYVEELKPTPEGDLEILLQKRENHECVEKTLMAQKTEDPAVFTPGPEMDDEAMEKFAGALASLLEHVQMVLGLRQGAELRSVTPAAAMKCLLLALALTCGAQALIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEGKPS

SEQ ID NO: 603 - protein, 178 aa, Bos indicus
  VARIANT 80: note = X is any amino acid
  VARIANT 134: note = X is any amino acid
MKCLLLALALTCGAQALIVTQTMKGLDIQKVAGTWYSLAMAASDISLLDAQSAPLRVYVEELKPTPEGDLEILLQKWENXECAQKKIIAEKTKIPAVFKIDALNENKVLVLDTDYKKYLLFCMENSAEPEQSLXCQCLVRTPEVDDEALEKFDKALKALPMHIRLSFNPTQLEEQCHI

SEQ ID NO: 604 - protein, 184 aa, Jeotgalicoccus coquinae
MTPAAAMKCLLLALALTCGAQALIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHI

SEQ ID NO: 605 - protein, 181 aa, Jeotgalicoccus schoeneichii
LRSVIPAAAMKCLLLALALTCGAQALIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTGVQDRCLEREQSPCAGHRLQKVPALLHGEQC

SEQ ID NO: 606 - protein, 178 aa, Bison bison
MKCLLLALALTCGAQALIVTQTMKGLDIQKVAGTWYSLAMAASDISLLDAQSAPLRVYVEELKPTPEGNLEILLQKWENGECAQKKIIAEKTKVPAVFKIDALNENKVLVLDTDYKKYLLFCMENSAEPEQSLACQCLVRTPEVDDEALEKFDKALKALPMHIRLSFNPTQLEGQCHI

SEQ ID NO: 607 - protein, 180 aa, unidentified
  REGION 1..180: note = Ovis sp.
MKCLLLALGLALACGVQAIIVTQTMKGLDIQKVAGTWYSLAMAASDISLLDAQSAPLRVYVEELKPTPEGNLEILLQKWENGECAQKKIIAEKTKIPAVFKIDALNENKVLVLDTDYKKYLLFCMENSAEPEQSLACQCLVRTPEVDNEALEKFDKALKALPMHIRLAFNPTQLEGQCHV

SEQ ID NO: 608 - protein, 162 aa, Ovis aries
IIVTQTMKGLDIQKVAGTWHSLAMAASDISLLDAQSAPLRVYVEELKPTPEGNLEILLQKWENGECAQKKIIAEKTKIPAVFKIDALNENKVLVLDTDYKKYLLFCMENSAEPEQSLACQCLVRTPEVDNEALEKFDKALKALPMHIRLAFNPTQLEGQCHV

SEQ ID NO: 609 - protein, 180 aa, Odocoileus virginianus
MKCLLLALGLALACGVQAAHIPQTAEDLDVRKVVGTWHVVAMAASDMSLLDAESGPLRVYVEELKPTPQGDLEVLLQKRENGKCAQKKIIAEKTEIPAVFKIDALNENKVLVLDTDYKKYLLFCMENSAEPEQSLACQCLVRTPEVDDEAMEKFDKALKALPMHIRLSFNPTQLEEQCRI

SEQ ID NO: 610 - protein, 162 aa, Rangifer tarandus
IIVTQTMKDLDVQKVAGTWYSLAMAASDISLLDAQSAPLRVYVEELKPTPGGDLEILLQKWENGKCAQKKIIAEKTEIPAVFKIDALNENKVLVLDTDYKKYLLFCMENSAEPEQSLACQCLVRTPEVDDEAMEKFDKALKALPMHIRLSFNPTQLEEQCRV

SEQ ID NO: 611 - protein, 180 aa, Rangifer tarandus
MKCLLITLGLALACGAQAIIVTQTMKDLDVQKVAGTWYSLAMAASDISLLDAQSAPLRVYVEELKPTPGGDLEILLQKWENGKCAQKKIIAEKTEIPAVFKIDALNENKVLVLDTDYKKYLLFCMENSAEPEQSLACQCLVRTPEVDDEAMEKFDKALKALPMHIRLSFNPTQLEEQCRV

SEQ ID NO: 612 - protein, 180 aa, Muntiacus muntjak
MKCLLLALGLALACGAQAIVVTQTMKDLDIQKVAGTWYSLAMAASDISLLDAQSAPLRVYVEELKPTPEGDLEILLQKWENGECAQKKITAEKTEIPAVFKIDALNENEVLVLDTDYEKYLLFCMDNSAEPEQSLACQCLVRTPEVDAEAMEKFDKALEALPMHIRLSFNPTQLEEQCHV

SEQ ID NO: 613 - protein, 180 aa, Muntiacus reevesi
MKCLLLALGLALACGAQAIIVTQTMKDLDIQKVAGTWYSLAMAASDISLLDAQSAPLRVYVEELKPTPEGDLEILLQKWENGECAQKKITAEKTEIPAVFKIDALNENNVLVLDTDYEKYLLFCMDNSAEPEQSLACQCLVRTPEVDAEAMEKFDKALEALPMHIRLSFNPTQLEEHCHV

SEQ ID NO: 614 - protein, 162 aa, Equus caballus
TNIPQTMQDLDLQEVAGTWYSLAMAASDISLLDSESAPLRVYVEELKPTPEGDLEILLQKWENKGCAQKKIIAEKTESPAEFKIDALDENKVLVLDTDYKNYLLFCMENAATPGQSLACQALVRTQMVDDEALEKFDKALQPLPMHIRLSFNPTRMAERCRI

SEQ ID NO: 615 - skipped

SEQ ID NO: 616 - protein, 4 aa, synthetic construct
  REGION 1..4: note = ER peptide tag
KDEL

SEQ ID NO: 617 - DNA, 1161 bp, synthetic construct
  misc_feature 1..1161: note = codon optimized ovalbumin
  source 1..1161: mol_type = other DNA
atgggatcaatcggtgctgccagcatggagttttgctttgatgtatttaaggaactcaaagtacatcacgctaacgaaaatattttctactgtcctatagctataatgtccgcacttgctatggtctatttgggcgccaaggattctacacgcacccagattaataaggtggttcgttttgacaaacttccaggctttggtgattcaatagaggcccaatgtgggacaagtgtcaacgtacacagctctttgcgtgatatactcaaccaaataactaaacccaatgacgtgtatagtttttcccttgcctcccgtctttatgctgaagaacgttacccaatattgcccgaatacctccaatgtgtcaaggaactgtatcgcggcggacttgaaccaataaattttcagaccgcagccgatcaggccagggagctcataaactcatgggtcgaaagtcaaacaaatgggatcatacgtaatgtgctccagccttctagcgttgattcacaaaccgccatggtgttggtcaatgccatcgtatttaaaggtctctgggaaaagacatttaaggatgaagatactcaggcaatgcctttccgtgtaaccgagcaggagtccaaacccgttcaaatgatgtaccagatagggttgtttagggtagccagtatggcctctgagaaaatgaagatactggaattgccctttgccagtggtaccatgtccatgcttgtactgttgccagatgaagtttctggcctggagcagcttgagtctataataaacttcgagaagttgacagagtggacatcatctaacgttatggaagaacgtaaaataaaagtgtatttgcctcgcatgaagatggaggagaaatacaaccttaccagtgtactgatggcaatgggcataaccgatgttttttctagttccgcaaacctttctggtatctcctcagcagaatctctgaagatatcccaagcagttcatgcagcacacgcagaaataaacgaggcaggacgtgaagtggtaggatcagccgaggcaggcgttgatgcagcatccgtgagcgaagagtttcgtgccgatcaccctttccttttctgcatcaaacacattgctaccaatgccgttcttttcttcgggcgctgtgtatccccttaa

SEQ ID NO: 618 - DNA, 2118 bp, synthetic construct
  misc_feature 1..2118: note = codon optimized ovotransferrin
  source 1..2118: mol_type = other DNA
atgaaactgattttgtgtaccgtcctgtcacttgggattgccgcagtatgttttgccgccccacccaagagtgttatccgttggtgtaccatctctagccctgaggagaagaaatgtaacaacctcagggacttgacccagcaagagaggataagcctgacatgtgtccagaaggctacctatctcgactgtatcaaagccatagccaacaacgaggccgacgcaatatcccttgatggaggacaggtgttcgaggccggacttgccccttataaattgaagcctatagctgctgaaatctacgagcatactgagggttctaccactagttattatgccgtagccgtagttaaaaaggggaccgagtttacagtcaacgatctccagggtaaaaatagctgccatactggtcttggtaggagtgctgggtggaatatacctatcggtacactcctccactggggcgctatcgagtgggagggtatcgaaagtggaagcgtggaacaggcagtcgctaagtttttttccgcctcttgcgtacccggcgctactatagagcagaaactttgccgtcagtgcaaaggagatcccaagaccaagtgtgcccgtaacgccccctatagtggatattccggcgctttccactgcttgaaggatggaaagggggacgtagccttcgtgaaacacactacagtgaatgaaaacgcccccgatctcaatgacgagtatgagttgctttgcctggatggtagtcgccaaccagtcgataattacaaaacctgtaactgggctcgtgttgctgcacatgccgtcgttgcacgcgatgacaataaagtggaggatatctggtcctttctgtcaaaagcccaaagcgattttggcgtagataccaagtcagatttccatctgtttgggccacctggaaagaaagaccctgtgcttaaggacttcttgttcaaagacagtgccataatgctcaaacgcgttcctagccttatggattctcaactgtatctcgggttcgaatattactcagccatacaatcaatgaggaaggaccagctcactcctagccctagagagaatagaattcaatggtgtgctgttggaaaagacgagaaatctaaatgcgaccgttggagcgtcgtcagcaacggtgatgttgagtgtactgtagttgatgaaactaaggattgcattattaagattatgaagggggaggccgatgccgtcgcattggatggagggctggtttataccgcaggtgtctgtggactggtgcctgtcatggcagaaagatatgatgatgaatcacaatgcagtaaaacagacgaacgtccagcctcatattttgctgtcgccgtcgcccgcaaggattccaatgtgaattggaataatttgaagggaaaaaaatcctgccacactgctgtagggcgtacagcaggttgggtgatcccaatgggactgattcacaataggaccggtacttgcaactttgatgaatatttctccgaggggtgtgctcccggtagcccccccaacagccgcttgtgccagctgtgccaaggtagtggaggtattccccctgagaaatgcgtcgcttcctctcacgagaaatactttggttatactggtgccttgcgttgcctcgtagaaaaaggtgacgtcgcttttatccaacacagtactgtagaggagaatacaggggggaagaacaaagctgactgggctaagaatcttcagatggacgatttcgaactgttgtgtaccgatggtagaagggctaatgttatggattatcgtgagtgcaatcttgcagaagtaccaacccatgctgtagttgtcagacccgagaaagcaaataaaattagagaccttttggagagacaagagaaacgtttcggtgtaaacggctcagagaagtctaaattcatgatgtttgaaagtcaaaataaggatctgttgttcaaagacttgactaaatgcctgtttaaagtcagggaaggcaccacttataaagaatttctgggagataaattctacacagtaataagcaacttgaaaacatgcaatccttccgatatcctgcaaatgtgcagtttccttgaagggaaataa

SEQ ID NO: 619 - DNA, 633 bp, synthetic construct
  misc_feature 1..633: note = codon optimized ovomucoid
  source 1..633: mol_type = other DNA
atggctatggccggagtctttgttcttttctctttcgtgctttgtggttttttgcctgacgccgccttcggagccgaggttgactgctctcgcttcccaaatgcaaccgataaagagggaaaggacgttcttgtttgcaacaaggacctgcgtcctatctgtgggacagatggcgttacatatactaacgactgccttttgtgtgcatattctattgaatttggtactaacatatctaaggagcatgatggggaatgcaaagaaacagttcccatgaactgtagttcttatgctaataccactagtgaggacggcaaggtcatggtattgtgcaatagggctttcaatcctgtatgcgggacagatggtgtcacttacgacaatgaatgtttgctgtgtgctcacaaggtcgaacaaggagcttctgtcgataagaggcatgatgggggttgcagaaaagaattggctgcagtatcagttgactgctctgagtatcccaagccagattgcaccgccgaggaccgcccattgtgtggaagcgataataagacttatggaaataaatgcaacttctgcaatgccgttgtggaaagcaacggcactctgactttgagtcatttcggaaaatgctga

SEQ ID NO: 620 - DNA, 444 bp, synthetic construct
  misc_feature 1..444: note = codon optimized lysozyme
  source 1..444: mol_type = other DNA
atgaggagcttgttgatacttgtgctttgtttccttccccttgcagcattggggaaagttttcggtaggtgcgagcttgccgccgcaatgaaaagacacggcttggacaattatcgtggatactctctcggcaattgggtttgtgtagcaaagttcgagagcaattttaacacccaggctactaatagaaataccgacggatctaccgactacgggattctgcaaataaacagccgctggtggtgtaatgacgggcgtactcccggtagccgcaatctctgtaacatcccctgtagtgcattgcttagttctgacattacagctagcgtgaactgtgctaaaaagatagtttctgacggtaatggaatgagtgcttgggttgcctggaggaaccgttgtaaggggaccgacgttcaagcatggattagagggtgtcgtctgtga

SEQ ID NO: 621 - DNA, 321 bp, synthetic construct
  misc_feature 1..321: note = codon optimized apovitellenin-1
  source 1..321: mol_type = other DNA
atggtacaatacagagcactcgtgattgccgtaattttgcttctttccactaccgtccctgaggtacatagcaagtccatcattgacagagaacgcagggactggctggtgattcctgatgctgctgctgcctatatttatgaagccgtcaacaaggtatcaccacgcgcaggtcagttcttgctcgacgtttctcaaaccacagtcgtgtctggaatcaggaactttctcatcaacgaaacagctaggcttactaagctggccgagcaacttatggagaaaattaagaacctttgctatactaaagtgttgggctactag

SEQ ID NO: 622 - protein, 386 aa, Gallus gallus
MGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKTFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 623 - protein, 705 aa, Gallus gallus
MKLILCTVLSLGIAAVCFAAPPKSVIRWCTISSPEEKKCNNLRDLTQQERISLTCVQKATYLDCIKAIANNEADAISLDGGQVFEAGLAPYKLKPIAAEIYEHTEGSTTSYYAVAVVKKGTEFTVNDLQGKNSCHTGLGRSAGWNIPIGTLLHWGAIEWEGIESGSVEQAVAKFFSASCVPGATIEQKLCRQCKGDPKTKCARNAPYSGYSGAFHCLKDGKGDVAFVKHTTVNENAPDLNDEYELLCLDGSRQPVDNYKTCNWARVAAHAVVARDDNKVEDIWSFLSKAQSDFGVDTKSDFHLFGPPGKKDPVLKDFLFKDSAIMLKRVPSLMDSQLYLGFEYYSAIQSMRKDQLTPSPRENRIQWCAVGKDEKSKCDRWSVVSNGDVECTVVDETKDCIIKIMKGEADAVALDGGLVYTAGVCGLVPVMAERYDDESQCSKTDERPASYFAVAVARKDSNVNWNNLKGKKSCHTAVGRTAGWVIPMGLIHNRTGTCNFDEYFSEGCAPGSPPNSRLCQLCQGSGGIPPEKCVASSHEKYFGYTGALRCLVEKGDVAFIQHSTVEENTGGKNKADWAKNLQMDDFELLCTDGRRANVMDYRECNLAEVPTHAVVVRPEKANKIRDLLERQEKRFGVNGSEKSKFMMFESQNKDLLFKDLTKCLFKVREGTTYKEFLGDKFYTVISNLKTCNPSDILQMCSFLEGK

SEQ ID NO: 624 - protein, 210 aa, Gallus gallus
MAMAGVFVLFSFVLCGFLPDAAFGAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 625 - protein, 147 aa, Gallus gallus
MRSLLILVLCFLPLAALGKVFGRCELAAAMKRHGLDNYRGYSLGNWVCVAKFESNFNTQATNRNTDGSTDYGILQINSRWWCNDGRTPGSRNLCNIPCSALLSSDITASVNCAKKIVSDGNGMSAWVAWRNRCKGTDVQAWIRGCRL

SEQ ID NO: 626 - protein, 106 aa, Gallus gallus
MVQYRALVIAVILLLSTTVPEVHSKSIIDRERRDWLVIPDAAAAYIYEAVNKVSPRAGQFLLDVSQTTVVSGIRNFLINETARLTKLAEQLMEKIKNLCYTKVLGY

SEQ ID NO: 627-628 - skipped (2 entries)

SEQ ID NO: 629 - RNA, 12 nt, synthetic construct
  misc_feature 1..12: note = ER peptide tag encoding sequence
  source 1..12: mol_type = other RNA
aaggatgagctt

SEQ ID NO: 630-631 - skipped (2 entries)

SEQ ID NO: 632 - protein, 4 aa, synthetic construct
  REGION 1..4: note = ER peptide tag
HDEF

SEQ ID NO: 633 - protein, 4 aa, synthetic construct
  REGION 1..4: note = ER peptide tag
HDEL

SEQ ID NO: 634 - protein, 4 aa, synthetic construct
  REGION 1..4: note = ER peptide tag
RDEF

SEQ ID NO: 635 - protein, 4 aa, synthetic construct
  REGION 1..4: note = ER peptide tag
RDEL

SEQ ID NO: 636 - protein, 4 aa, synthetic construct
  REGION 1..4: note = ER peptide tag
WDEL

SEQ ID NO: 637 - protein, 4 aa, synthetic construct
  REGION 1..4: note = ER peptide tag
YDEL

SEQ ID NO: 638 - protein, 4 aa, synthetic construct
  REGION 1..4: note = ER peptide tag
HEEF

SEQ ID NO: 639 - protein, 4 aa, synthetic construct
  REGION 1..4: note = ER peptide tag
HEEL

SEQ ID NO: 640 - protein, 4 aa, synthetic construct
  REGION 1..4: note = ER peptide tag
KEEL

SEQ ID NO: 641 - protein, 4 aa, synthetic construct
  REGION 1..4: note = ER peptide tag
REEL

SEQ ID NO: 642 - protein, 4 aa, synthetic construct
  REGION 1..4: note = ER peptide tag
KAEL

SEQ ID NO: 643 - protein, 4 aa, synthetic construct
  REGION 1..4: note = ER peptide tag
KCEL

SEQ ID NO: 644 - protein, 4 aa, synthetic construct
  REGION 1..4: note = ER peptide tag
KFEL

SEQ ID NO: 645 - protein, 4 aa, synthetic construct
  REGION 1..4: note = ER peptide tag
KGEL

SEQ ID NO: 646 - protein, 4 aa, synthetic construct
  REGION 1..4: note = ER peptide tag
KHEL

SEQ ID NO: 647 - protein, 4 aa, synthetic construct
  REGION 1..4: note = ER peptide tag
KLEL

SEQ ID NO: 648 - protein, 4 aa, synthetic construct
  REGION 1..4: note = ER peptide tag
KNEL

SEQ ID NO: 649 - protein, 4 aa, synthetic construct
  REGION 1..4: note = ER peptide tag
KQEL

SEQ ID NO: 650 - protein, 4 aa, synthetic construct
  REGION 1..4: note = ER peptide tag
KREL

SEQ ID NO: 651 - protein, 4 aa, synthetic construct
  REGION 1..4: note = ER peptide tag
KSEL

SEQ ID NO: 652 - protein, 4 aa, synthetic construct
  REGION 1..4: note = ER peptide tag
KVEL

SEQ ID NO: 653 - protein, 4 aa, synthetic construct
  REGION 1..4: note = ER peptide tag
KWEL

SEQ ID NO: 654 - protein, 4 aa, synthetic construct
  REGION 1..4: note = ER peptide tag
KYEL

SEQ ID NO: 655 - protein, 4 aa, synthetic construct
  REGION 1..4: note = ER peptide tag
KEDL

SEQ ID NO: 656 - protein, 4 aa, synthetic construct
  REGION 1..4: note = ER peptide tag
KIEL

SEQ ID NO: 657 - protein, 4 aa, synthetic construct
  REGION 1..4: note = ER peptide tag
DKEL

SEQ ID NO: 658 - protein, 4 aa, synthetic construct
  REGION 1..4: note = ER peptide tag
FDEL

SEQ ID NO: 659 - protein, 4 aa, synthetic construct
  REGION 1..4: note = ER peptide tag
KDEF

SEQ ID NO: 660 - protein, 4 aa, synthetic construct
  REGION 1..4: note = ER peptide tag
KKEL

SEQ ID NO: 661 - protein, 4 aa, synthetic construct
  REGION 1..4: note = ER peptide tag
HADL

SEQ ID NO: 662 - protein, 4 aa, synthetic construct
  REGION 1..4: note = ER peptide tag
HAEL

SEQ ID NO: 663 - protein, 4 aa, synthetic construct
  REGION 1..4: note = ER peptide tag
HIEL

SEQ ID NO: 664 - protein, 4 aa, synthetic construct
  REGION 1..4: note = ER peptide tag
HNEL

SEQ ID NO: 665 - protein, 4 aa, synthetic construct
  REGION 1..4: note = ER peptide tag
HTEL

SEQ ID NO: 666 - protein, 4 aa, synthetic construct
  REGION 1..4: note = ER peptide tag
KTEL

SEQ ID NO: 667 - protein, 4 aa, synthetic construct
  REGION 1..4: note = ER peptide tag
HVEL

SEQ ID NO: 668 - protein, 4 aa, synthetic construct
  REGION 1..4: note = ER peptide tag
NDEL

SEQ ID NO: 669 - protein, 4 aa, synthetic construct
  REGION 1..4: note = ER peptide tag
QDEL

SEQ ID NO: 670 - protein, 4 aa, synthetic construct
  REGION 1..4: note = ER peptide tag
REDL

SEQ ID NO: 671 - protein, 4 aa, synthetic construct
  REGION 1..4: note = ER peptide tag
RNEL

SEQ ID NO: 672 - protein, 4 aa, synthetic construct
  REGION 1..4: note = ER peptide tag
RTDL

SEQ ID NO: 673 - protein, 4 aa, synthetic construct
  REGION 1..4: note = ER peptide tag
RTEL

SEQ ID NO: 674 - protein, 4 aa, synthetic construct
  REGION 1..4: note = ER peptide tag
SDEL

SEQ ID NO: 675 - protein, 4 aa, synthetic construct
  REGION 1..4: note = ER peptide tag
TDEL

SEQ ID NO: 676 - protein, 4 aa, synthetic construct
  REGION 1..4: note = ER peptide tag
SKEL

SEQ ID NO: 677 - protein, 4 aa, synthetic construct
  REGION 1..4: note = ER peptide tag
STEL

SEQ ID NO: 678 - protein, 4 aa, synthetic construct
  REGION 1..4: note = ER peptide tag
EDEL

SEQ ID NO: 679 - DNA, 770 bp, unidentified
  misc_feature 1..770: note = Intron 1
  source 1..770: mol_type = genomic DNA
gttcgttatctaccaccgttctatggattttattccttctattcgtgtttattctattggtttatgttgcttgcaatatgttttttctgaatctgtcgtcgttgtcttcaattttatccatgtttcagagatcaattttgtttgtgtagtatgtgcttattcttcttcttttcgttcgagttgttaataacggtgctatggtgttttcaaaagtgttttttttattacttttgatttaaagtttttttggtaaggcttttatttgcttgttatattcaaatctttggatccagatcttatataagtttttggttcaagaaagtttttggttactgatgaatagatctattaactgttactttaatcgattcaagctaaagttttttggttactgatgaatagatctattatctgttacttttaatcggttcaagctcaagttttttggttactgatgaatagatctatatacgtcacagtgtgctaaacatgcccttgttttatctcgatcttatgtatgggagtgccataaattttgttatgtctatttttttatctgttggaatcatactgagtttgatgcgttacgattgagcatacctatttttgggcttgttgtatggtgggtatttagatcttaatctttttatgcttatgaaaggttttgtaatgacaaaggtcttaatgttgttaaacttttatttttactttatatggtgtgttgatgtgttatggttttgacaactttttttttttctggatttttgcag

SEQ ID NO: 680 - DNA, 93 bp, unidentified
  misc_feature 1..93: note = Intron 2
  source 1..93: mol_type = genomic DNA
gtaaccatatctttcatctgttatgtgactacacattgcttctctttttgtgttctgtctcattaattgcggtttgttacatgttgtttgtag

SEQ ID NO: 681 - DNA, 85 bp, unidentified
  misc_feature 1..85: note = Intron 3
  source 1..85: mol_type = genomic DNA
gtaagcaaccaacacaccatctaatacgctagcaaattcaatattatcattatccttatatttgtttccgcgcttgattttatag

SEQ ID NO: 682 - DNA, 269 bp, unidentified
  misc_feature 1..269: note = Intron 4
  source 1..269: mol_type = genomic DNA
gtttgtatttactcaaatgttgatcagtagtgtttttaggacattgattaagaaacccaaaaaataattatttttattgaaacgcataaatttatactagccgtgactgtttttatgtccttatatgatcttcgcaatatatattttctattataagtttcttaaccaatgcactaacttactgttaacaagaccttattattaaacatcatctatcacttggttaattgtattcatttgatgcatggtaatgcattacatatatacag

SEQ ID NO: 683 - DNA, 486 bp, synthetic construct; identical to SEQ ID NO: 9
  misc_feature 1..486: note = OLG1
  source 1..486: mol_type = other DNA

SEQ ID NO: 684 - protein, 162 aa, synthetic construct; identical to SEQ ID NO: 10
  REGION 1..162: note = OLG1

SEQ ID NO: 685 - DNA, 486 bp, synthetic construct; identical to SEQ ID NO: 11
  misc_feature 1..486: note = OLG2
  source 1..486: mol_type = other DNA

SEQ ID NO: 686 - protein, 162 aa, synthetic construct
  REGION 1..162: note = OLG2
LIVTQTMKGLDIQKVAGTWYSLAMAASDISLLDAQSAPLRVYVEELKPTPEGDLEILLQKWENDECAQKKIIAEKTKIPAVFKIDALNENKVLVLDTDYKKYLLVCMENSAEPEQSLVCQCLVRTPEVDDEALEKFDKALKALPMHIRLSFNPTQLEEQCHI

SEQ ID NO: 687 - DNA, 486 bp, synthetic construct; identical to SEQ ID NO: 12
  misc_feature 1..486: note = OLG3
  source 1..486: mol_type = other DNA

SEQ ID NO: 688 - protein, 162 aa, synthetic construct; identical to SEQ ID NO: 686
  REGION 1..162: note = OLG3

SEQ ID NO: 689 - DNA, 486 bp, synthetic construct; identical to SEQ ID NO: 13
  misc_feature 1..486: note = OLG4
  source 1..486: mol_type = other DNA

SEQ ID NO: 690 - protein, 162 aa, synthetic construct; identical to SEQ ID NO: 686
  REGION 1..162: note = OLG4

SEQ ID NO: 691 - DNA, 1256 bp, synthetic construct
  misc_feature 1..1256: note = OLG2 (intron 1)
  source 1..1256: mol_type = other DNA
cttattgtgacccaaaccatgaagggcctcgacattcaaaaggttcgttatctaccaccgttctatggattttattccttctattcgtgtttattctattggtttatgttgcttgcaatatgttttttctgaatctgtcgtcgttgtcttcaattttatccatgtttcagagatcaattttgtttgtgtagtatgtgcttattcttcttcttttcgttcgagttgttaataacggtgctatggtgttttcaaaagtgttttttttattacttttgatttaaagtttttttggtaaggcttttatttgcttgttatattcaaatctttggatccagatcttatataagtttttggttcaagaaagtttttggttactgatgaatagatctattaactgttactttaatcgattcaagctaaagttttttggttactgatgaatagatctattatctgttacttttaatcggttcaagctcaagttttttggttactgatgaatagatctatatacgtcacagtgtgctaaacatgcccttgttttatctcgatcttatgtatgggagtgccataaattttgttatgtctatttttttatctgttggaatcatactgagtttgatgcgttacgattgagcatacctatttttgggcttgttgtatggtgggtatttagatcttaatctttttatgcttatgaaaggttttgtaatgacaaaggtcttaatgttgttaaacttttatttttactttatatggtgtgttgatgtgttatggttttgacaactttttttttttctggatttttgcaggttgccggaacctggtactcccttgctatggctgcttccgatatctccttgctcgatgctcaatccgctccacttagggtgtacgtggaagagttgaagccaactccagagggcgatcttgagatcttgcttcaaaagtgggagaacgatgagtgcgcccagaagaagattatcgccgaaaagaccaagattcccgccgtgttcaagatcgatgctctcaacgagaacaaggtgctcgtgctcgataccgactacaagaagtaccttctcgtctgcatggaaaactccgctgagccagagcaatctcttgtttgccaatgccttgtgaggaccccagaggttgacgatgaagctcttgagaagttcgacaaggctctcaaggctttgcctatgcacatccgccttagcttcaacccaactcagcttgaggaacagtgccacatc

SEQ ID NO: 692 - DNA, 579 bp, synthetic construct
  misc_feature 1..579: note = OLG2 (intron 2)
  source 1..579: mol_type = other DNA
cttattgtgacccaaaccatgaagggcctcgacattcaaaaggtaaccatatctttcatctgttatgtgactacacattgcttctctttttgtgttctgtctcattaattgcggtttgttacatgttgtttgtaggttgccggaacctggtactcccttgctatggctgcttccgatatctccttgctcgatgctcaatccgctccacttagggtgtacgtggaagagttgaagccaactccagagggcgatcttgagatcttgcttcaaaagtgggagaacgatgagtgcgcccagaagaagattatcgccgaaaagaccaagattcccgccgtgttcaagatcgatgctctcaacgagaacaaggtgctcgtgctcgataccgactacaagaagtaccttctcgtctgcatggaaaactccgctgagccagagcaatctcttgtttgccaatgccttgtgaggaccccagaggttgacgatgaagctcttgagaagttcgacaaggctctcaaggctttgcctatgcacatccgccttagcttcaacccaactcagcttgaggaacagtgccacatc

SEQ ID NO: 693 - DNA, 1155 bp, synthetic construct
  misc_feature 1..1155: note = OOVAL1
  source 1..1155: mol_type = other DNA
ggtagcattggggctgcttctatggaattttgtttcgatgtctttaaagaacttaaggtacaccatgcaaatgagaacattttctactgtcccatcgctataatgtctgcacttgcaatggtttaccttggggctaaagacagtactcgtacacaaataaataaagtagtgagattcgataagttgcctgggttcggggattctatcgaagctcaatgtgggaccagtgttaacgtacatagctccttgcgcgatatcttgaatcaaataacaaagcctaatgatgtatactcattttcattggcctctcgcttgtatgccgaggaaagataccccattctgccagaataccttcagtgcgtcaaggaactctaccgcggaggactcgagcccataaatttccagactgcagcagaccaggccagggagctgattaactcttgggtagagagccagacaaatggcataatcaggaatgtgctgcagccatcatcagttgattcacaaacagctatggtgctggttaatgcaatcgtcttcaaagggttgtgggaaaaggcttttaaggacgaagatactcaagctatgcctttccgtgtaacagagcaagaaagcaagcctgtacaaatgatgtatcagattggtctgtttcgtgttgcctctatggcttcagagaaaatgaagatactcgaacttcccttcgcatcagggactatgagcatgttggttttgttgcctgatgaggtatctggtttggaacagctggaatcaataatcaatttcgagaagttgacagaatggaccagttctaatgttatggaagagcgtaagataaaagtatatttgcctcgtatgaaaatggaagaaaagtacaatttgaccagcgttttgatggctatgggcatcactgacgttttttcatcttctgctaatctcagcggcatatccagcgcagagagcctcaaaatatcccaagccgtccatgctgcacatgcagagataaatgaggctggtagggaagtggtcgggagcgctgaagctggggtagatgcagccagtgtaagtgaagagttcagggctgaccatcccttcctgttctgcattaagcacattgcaactaacgcagtactcttttttggacgttgcgtgagcccc

SEQ ID NO: 694 - protein, 385 aa, synthetic construct
  REGION 1..385: note = OOVAL1
GSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 695 - DNA, 1155 bp, synthetic construct
  misc_feature 1..1155: note = OOVAL2
  source 1..1155: mol_type = other DNA
ggatcaattggcgccgcatctatggagttctgcttcgatgtttttaaagagcttaaagtgcaccatgccaacgagaatatcttctattgcccaattgccattatgtctgcccttgctatggtgtacttgggtgctaaagactctactaggacccagataaacaaggtagtcagattcgacaagctgcctgggtttggcgactctattgaagctcagtgtggtacttctgttaatgtccactcatccctccgcgacatacttaatcaaattacaaaaccaaatgatgtgtactcatttagtctggccagccgtttgtacgcagaggaacgctaccctatcctgccagagtatttgcaatgtgtgaaggaactttacaggggtgggcttgagccaataaactttcaaacagcagccgaccaagctagggagcttatcaattcttgggtcgagagccaaactaacggaatcatccgcaacgtcctccagccaagttccgttgattcccagaccgctatggtacttgtgaatgccattgtcttcaaggggctttgggagaaggcatttaaagacgaggacactcaggcaatgccctttcgtgtgaccgagcaggagtcaaaacctgttcaaatgatgtaccaaattgggctgttcagagttgctagtatggcctctgagaaaatgaagatccttgaactcccatttgcctccgggacaatgtctatgcttgtcctcctgccagatgaagtcagtgggctcgaacagctcgaaagcataataaactttgagaaacttaccgaatggacttcttccaatgttatggaggagcgtaaaattaaggtctatctgccccgcatgaaaatggaggaaaagtataatctcactagcgtcctcatggctatgggaattactgatgtattctcctctagcgctaatctgagtggaatctccagcgccgagtctctcaagataagccaggccgtgcacgctgctcatgctgaaatcaacgaagccggcagagaggtggtggggtcagctgaggcaggtgtagatgcagccagtgtctctgaggaatttagagccgatcaccctttccttttttgcattaaacatatcgctacaaatgccgttttgtttttcggtcgttgcgttagtcca

SEQ ID NO: 696 - protein, 385 aa, synthetic construct; identical to SEQ ID NO: 694
  REGION 1..385: note = OOVAL2

SEQ ID NO: 697 - DNA, 1155 bp, synthetic construct
  misc_feature 1..1155: note = OOVAL3
  source 1..1155: mol_type = other DNA
gggtccatcggtgccgcctcaatggaattctgctttgacgtcttcaaggaacttaaggtacatcatgccaacgagaatattttttactgtccaatagctatcatgagtgcacttgctatggtgtaccttggagccaaagactcaacccgtacccagatcaacaaggtggtccgctttgacaaactgccagggtttggggattctattgaggcccaatgcggaacaagtgtgaacgtccactctagcttgcgcgatatacttaatcaaataactaaaccaaatgatgtgtattcattctctctcgccagcagactgtacgcagaagaaaggtatcccattctccccgagtacctccaatgcgtaaaggagttgtacagaggcggcctggaacccataaatttccaaactgccgcagatcaggctcgtgagctgataaattcatgggtcgagagccaaactaacggtatcattcgtaatgtccttcaaccctcaagtgtggacagtcagacagccatggtcctcgtcaatgctatagtcttcaaaggcctgtgggaaaagacctttaaggatgaagatactcaagcaatgccctttagagtcacagagcaagaaagcaaacccgtgcaaatgatgtatcaaatcgggctctttcgtgttgcatccatggcatctgaaaagatgaagatattggaactccccttcgcctctggaacaatgagtatgttggtacttctgcccgatgaggtctctgggttggaacagcttgaatctattattaacttcgagaaactgaccgagtggactagtagtaatgtcatggaggagagaaagattaaggtttatttgccacgcatgaagatggaagagaaatataacttgacatctgtactgatggcaatgggtataaccgacgtatttagcagtagcgccaatctgtcagggatttcttcagccgaaagtctcaagatttctcaggcagttcacgcagcccatgcagagataaacgaagcaggccgcgaagttgtcggatctgcagaagccggcgtggatgcagccagtgtctccgaagagttcagagcagaccaccctttcctcttctgcattaagcacatcgcaaccaacgcagtactttttttcggacgttgcgtgtcccca

SEQ ID NO: 698 - protein, 385 aa, synthetic construct; identical to SEQ ID NO: 694
  REGION 1..385: note = OOVAL3

SEQ ID NO: 699 - DNA, 1155 bp, synthetic construct
  misc_feature 1..1155: note = OOVAL4
  source 1..1155: mol_type = other DNA
ggttcaataggagctgcgtctatggagttttgttttgatgtctttaaggaactcaaagtccaccacgccaatgaaaatattttctattgccctattgcaatcatgagtgcgctagccatggtttacttgggtgcaaaagacagtacgcgtactcaaataaacaaggttgttcgctttgacaagcttcctggatttggagatagtattgaagcacaatgtggaactagcgtaaacgtccacagctcattgagggacattcttaaccaaattaccaagccaaatgatgtatatagtttttccttggcatcacgactgtatgcagaagaaagatatcctatcctcccggaatatcttcagtgcgtgaaagaattatacagaggtgggctagagccaatcaattttcaaaccgctgctgatcaagctcgcgagttgattaactcatgggttgagagccagacaaatgggataataagaaatgttcttcaaccatctagtgtggactctcaaacagcaatggtgctcgtcaatgcgatagtttttaaaggcttgtgggagaaaacattcaaagatgaggatactcaggcaatgccattccgtgtaactgaacaggaatctaagcctgttcaaatgatgtatcagattggtttgttcagagttgcctctatggcatctgaaaaaatgaaaattttggagcttccatttgctagtggaacaatgtcaatgttagttttactgcctgatgaagtgtccggtttagaacaattggaatcaattatcaactttgaaaagttgaccgagtggacttcctccaatgtgatggaggagaggaagattaaggtgtaccttcccagaatgaagatggaagagaaatataacctgacttcggtcctaatggctatggggatcacagatgtgttttcttcctcggcaaacctttcaggcatatcaagcgccgagtcattgaaaatttcacaggctgttcatgcagctcatgctgaaatcaatgaggccgggcgggaggttgtgggcagtgctgaagctggagttgatgctgcctcagtgtctgaggaatttagagcagatcatcctttcctcttctgcattaagcatattgctaccaatgctgtactgttcttcggtaggtgtgttagcccc

SEQ ID NO: 700 - protein, 385 aa, synthetic construct
  REGION 1..385: note = OOVAL4
GSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKTFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 701 - DNA, 1925 bp, synthetic construct
  misc_feature 1..1925: note = OOVAL2 (intron 1)
  source 1..1925: mol_type = other DNA
ggatcaattggcgccgcatctatggagttctgcttcgatgtttttaaagagcttaaagtgcaccatgccaacgagaatatcttctattgcccaattgccattatgtctgcccttgctatggtgtacttgggtgctaaagactctactaggacccagataaacaaggttcgttatctaccaccgttctatggattttattccttctattcgtgtttattctattggtttatgttgcttgcaatatgttttttctgaatctgtcgtcgttgtcttcaattttatccatgtttcagagatcaattttgtttgtgtagtatgtgcttattcttcttcttttcgttcgagttgttaataacggtgctatggtgttttcaaaagtgttttttttattacttttgatttaaagtttttttggtaaggcttttatttgcttgttatattcaaatctttggatccagatcttatataagtttttggttcaagaaagtttttggttactgatgaatagatctattaactgttactttaatcgattcaagctaaagttttttggttactgatgaatagatctattatctgttacttttaatcggttcaagctcaagttttttggttactgatgaatagatctatatacgtcacagtgtgctaaacatgcccttgttttatctcgatcttatgtatgggagtgccataaattttgttatgtctatttttttatctgttggaatcatactgagtttgatgcgttacgattgagcatacctatttttgggcttgttgtatggtgggtatttagatcttaatctttttatgcttatgaaaggttttgtaatgacaaaggtcttaatgttgttaaacttttatttttactttatatggtgtgttgatgtgttatggttttgacaactttttttttttctggatttttgcaggtagtcagattcgacaagctgcctgggtttggcgactctattgaagctcagtgtggtacttctgttaatgtccactcatccctccgcgacatacttaatcaaattacaaaaccaaatgatgtgtactcatttagtctggccagccgtttgtacgcagaggaacgctaccctatcctgccagagtatttgcaatgtgtgaaggaactttacaggggtgggcttgagccaataaactttcaaacagcagccgaccaagctagggagcttatcaattcttgggtcgagagccaaactaacggaatcatccgcaacgtcctccagccaagttccgttgattcccagaccgctatggtacttgtgaatgccattgtcttcaaggggctttgggagaaggcatttaaagacgaggacactcaggcaatgccctttcgtgtgaccgagcaggagtcaaaacctgttcaaatgatgtaccaaattgggctgttcagagttgctagtatggcctctgagaaaatgaagatccttgaactcccatttgcctccgggacaatgtctatgcttgtcctcctgccagatgaagtcagtgggctcgaacagctcgaaagcataataaactttgagaaacttaccgaatggacttcttccaatgttatggaggagcgtaaaattaaggtctatctgccccgcatgaaaatggaggaaaagtataatctcactagcgtcctcatggctatgggaattactgatgtattctcctctagcgctaatctgagtggaatctccagcgccgagtctctcaagataagccaggccgtgcacgctgctcatgctgaaatcaacgaagccggcagagaggtggtggggtcagctgaggcaggtgtagatgcagccagtgtctctgaggaatttagagccgatcaccctttccttttttgcattaaacatatcgctacaaatgccgttttgtttttcggtcgttgcgttagtcca

SEQ ID NO: 702 - DNA, 1248 bp, synthetic construct
  misc_feature 1..1248: note = OOVAL2 (intron 2)
  source 1..1248: mol_type = other DNA
ggatcaattggcgccgcatctatggagttctgcttcgatgtttttaaagagcttaaagtgcaccatgccaacgagaatatcttctattgcccaattgccattatgtctgcccttgctatggtgtacttgggtgctaaagactctactaggacccagataaacaaggtaaccatatctttcatctgttatgtgactacacattgcttctctttttgtgttctgtctcattaattgcggtttgttacatgttgtttgtaggtagtcagattcgacaagctgcctgggtttggcgactctattgaagctcagtgtggtacttctgttaatgtccactcatccctccgcgacatacttaatcaaattacaaaaccaaatgatgtgtactcatttagtctggccagccgtttgtacgcagaggaacgctaccctatcctgccagagtatttgcaatgtgtgaaggaactttacaggggtgggcttgagccaataaactttcaaacagcagccgaccaagctagggagcttatcaattcttgggtcgagagccaaactaacggaatcatccgcaacgtcctccagccaagttccgttgattcccagaccgctatggtacttgtgaatgccattgtcttcaaggggctttgggagaaggcatttaaagacgaggacactcaggcaatgccctttcgtgtgaccgagcaggagtcaaaacctgttcaaatgatgtaccaaattgggctgttcagagttgctagtatggcctctgagaaaatgaagatccttgaactcccatttgcctccgggacaatgtctatgcttgtcctcctgccagatgaagtcagtgggctcgaacagctcgaaagcataataaactttgagaaacttaccgaatggacttcttccaatgttatggaggagcgtaaaattaaggtctatctgccccgcatgaaaatggaggaaaagtataatctcactagcgtcctcatggctatgggaattactgatgtattctcctctagcgctaatctgagtggaatctccagcgccgagtctctcaagataagccaggccgtgcacgctgctcatgctgaaatcaacgaagccggcagagaggtggtggggtcagctgaggcaggtgtagatgcagccagtgtctctgaggaatttagagccgatcaccctttccttttttgcattaaacatatcgctacaaatgccgttttgtttttcggtcgttgcgttagtcca

SEQ ID NO: 703 - DNA, 1384 bp, unidentified
  misc_feature 1..1384: note = GmSeed2 promoter
  source 1..1384: mol_type = genomic DNA
aacacaagcttcaagttttaaaaggaaaaatgtcagccaaaaactttaaataaaatggtaacaaggaaattattcaaaaattacaaacctcgtcaaaataggaaagaaaaaaagtttagggatttagaaaaaacatcaatctagttccaccttattttatagagagaagaaactaatatataagaactaaaaaacagaagaatagaaaaaaaaagtattgacaggaaagaaaaagtagctgtatgcttataagtactttgaggatttgaattctctcttataaaacacaaacacaatttttagattttatttaaataatcatcaatccgattataattatttatatatttttctattttcaaagaagtaaatcatgagcttttccaactcaacatctattttttttctctcaacctttttcacatcttaagtagtctcaccctttatatatataacttatttcttaccttttacattatgtaacttttatcaccaaaaccaacaactttaaaattttattaaatagactccacaagtaacttgacactcttacattcatcgacattaacttttatctgttttataaatattattgtgatataatttaatcaaaataaccacaaactttcataaaaggttcttattaagcatggcatttaataagcaaaaacaactcaatcactttcatataggaggtagcctaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttcacaactcaaacattctctccattggtccttaaacactcatcagtcatcacc

SEQ ID NO: 704 - DNA, 1454 bp, unidentified
  misc_feature 1..1454: note = GmSeed12 promoter
  source 1..1454: mol_type = genomic DNA
caactattatcgcatgatgatgtacgttaagtcatcatcatctttaactttatatattgttaaaagtagaaaaaataggtgatgcattataaaataattttataacatcatttaattataaattatttataataaatatttgagtttttatagtaattacctaaacaattatatcaagactaatgcctgattagttgacatgacgaaattaaactcataaaagtaaagatgtttatgtggaaaactcttatacaattgagcggacttttttccatggtagttcagttttcttctattcaatttatttttttggtttccgctcagaataagaataatttgataaattcatttttaggcaattaagaatatttatttgactaactttttaattgaaataaatttacaataaatactcaatttatctttcacaatcaaaagattgagatgttgtaagatctccgataatatacttatatcttttcatttattacgttttcaaatttgaattttaatgtgtgttgtaagtataaatttaaaataaaaataaaaacaattattatatcaaaatggcaaaaacatttaatacgtattatttaagaaaaaaatatgtaataatatatttatattttaatatctattcttatgtattttttaaaaatctattatatattgatcaactaaaatatttttatatctacacttattttgcatttttatcaattttcttgcgttttttggcatatttaataatgactattctttaataatcaatcattattcttacatggtacatattgttggaaccatatgaagtgtccattgcatttgactatgtggatagtgttttgatccaggcctccatttgccgcttattaattaatttggtaacagtccgtactaatcagttacttatccttcctccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagcccacacatttgttgtttctaacccaacctcaaactcgtattctcttccgccacctcatttttgtttatttcaacacccgtcaaactgcatgccaccccgtggccaaatgtccatgcatgttaacaagacctatgactataaatatctgcaatctcggcccaggttttcatcatcaagaaccagttcaatatcctagtacaccgtattaaagaatttaagatatact

SEQ ID NO: 705 - DNA, 1543 bp, unidentified
  misc_feature 1..1543: note = PvPhas promoter
  source 1..1543: mol_type = genomic DNA
cattgtactcccagtatcattatagtgaaagttttggctctctcgccggtggttttttacctctatttaaaggggttttccacctaaaaattctggtatcattctcactttacttgttactttaatttctcataatctttggttgaaattatcacgcttccgcacacgatatccctacaaatttattatttgttaaacattttcaaaccgcataaaattttatgaagtcccgtctatctttaatgtagtctaacattttcatattgaaatatataatttacttaattttagcgttggtagaaagcataatgatttattcttattcttcttcatataaatgtttaatatacaatataaacaaattctttaccttaagaaggatttcccattttatattttaaaaatatatttatcaaatatttttcaaccacgtaaatcacataataataagttgtttcaaaagtaataaaatttaactccataatttttttatttgactgatcttaaagcaacacccagtgacacaactagccatttttttctttgaataaaaaaatccaattatcattgtattttttttatacaatgaaaatttcaccaaacaatgatttgtggtatttctgaagcaagtcatgttatgcaaaattctataattcccatttgacactacggaagtaactgaagatctgcttttacatgcgagacacatcttctaaagtaattttaataatagttactatattcaagatttcatatatcaaatactcaatattacttctaaaaaattaattagatataattaaaatattacttttttaattttaagtttaattgttgaatttgtgactattgatttattattctactatgtttaaattgttttataggtagtttaaagtaaatataagtaatgtagtagagtgttagagtgttaccctaaaccataaactataagatttatggtggactaattttcatatatttcttattgcttttaccttttcttggtatgtaagtccgtaactggaattactgtgggttgccatgacactctgtggtcttttggttcatgcatggatgcttgcgcaagaaaaagacaaagaacaaagaaaaaagacaaaacagagagacaaaacgcaatcacacaaccaactcaaattagtcactggctgatcaagatcgccgcgtccatgtatgtctaaatgccatgcaaagcaacacgtgcttaacatgcactttaaatggctcacccatcccaacccactcacaaacacattgcctttttcttcatcatcaccacaaccacctgtatatattcattctcttccgccacctcaatttcttcacttcaacacacgtcaacctgcatatgcgtgtcatcccatgcccaaatctccatgcatgttcctaccaccttctctcttatataatacctataaatacctctaatatcactcacttctttcatcatccatccatccagagtactactactctactactataataccccaacccaactcatattcaatactactctact

SEQ ID NO: 706 - DNA, 1130 bp, unidentified
  misc_feature 1..1130: note = BnNap promoter
  source 1..1130: mol_type = genomic DNA
catcggtgattgattcctttaaagacttatgtttcttatcttgcttctgaggcaagtattcagttaccagttaccacttatattctggactttctgactgcatcctcatttttccaacattttaaatttcactattggctgaatgcttcttctttgaggaagaaacaattcagatggcagaaatgtatcaaccaatgcatatatacaaatgtacctcttgttctcaaaacatctatcggatggttccatttgctttgtcatccaattagtgactactttatattattcactcctctttattactattttcatgcgaggttgccatgtacattatatttgtaaggattgacgctattgagcgtttttcttcaattttctttattttagacatgggtatgaaatgtgtgttagagttgggttgaatgagatatacgttcaagtgaagtggcataccgttgtcgagtaaggatgacctacccattcttgagacaaatgttacattttagtatcagagtaaaatgtgtacctataactcaaattcgattgacatgtatccattcaacataaaattaaaccagcctgcacctgcatccacatttcaagtattttcaaaccgttcggctcctatccaccgggtgtaacaagacggattccgaatttggaagattttgactcaaattcccaatttatattgaccgtgactaaatcaactttaacttctataattctgattaagctcccaatttatattcccaacggcactacctccaaaatttatagactctcatccccttttaaaccaacttagtaaacgtttttttttttaattttatgaagttaagtttttaccttgtttttaaaaagaatcgttcataagatgccatgccagaacattagctacacgttacacatagcatgcagccgcggagaattgtttttcttcgccacttgtcactcccttcaaacacctaagagcttctctctcacagcacacacatacaatcacatgcgtgcatgcattattacacgtgatcgccatgcaaatctcctttatagcctataaattaactcatccgcttcactctttactcaaaccaaaactcatcaatacaaacaagattaaaaacata

SEQ ID NO: 707 - DNA, 57 bp, unidentified
  misc_feature 1..57: note = sig2 signal peptide encoding sequence
  source 1..57: mol_type = other DNA
atggccaagctagttttttccctttgttttctgcttttcagtggctgctgcttcgct

SEQ ID NO: 708 - protein, 19 aa, unidentified
  REGION 1..19: note = sig2 signal peptide
MAKLVFSLCFLLFSGCCFA

SEQ ID NO: 709 - DNA, 96 bp, unidentified
  misc_feature 1..96: note = sig10 signal peptide encoding sequence
  source 1..96: mol_type = other DNA
atggctacttcaaagttgaaaacccagaatgtggttgtatctctctccctaaccttaaccttggtactggtgctactgaccagcaaggcaaactca

SEQ ID NO: 710 - protein, 32 aa, unidentified
  REGION 1..32: note = sig10 signal peptide
MATSKLKTQNVVVSLSLTLTLVLVLLTSKANS

SEQ ID NO: 711 - DNA, 66 bp, unidentified
  misc_feature 1..66: note = sig11 signal peptide encoding sequence
  source 1..66: mol_type = other DNA
atgatgagagcacggttcccattactgttgctgggacttgttttcctggcttcagtttctgtctca

SEQ ID NO: 712 - protein, 22 aa, unidentified
  REGION 1..22: note = sig11 signal peptide
MMRARFPLLLLGLVFLASVSVS

SEQ ID NO: 713 - DNA, 72 bp, unidentified
  misc_feature 1..72: note = sig12 signal peptide encoding sequence
  source 1..72: mol_type = other DNA
atgatgagagcgcggttcccattactgttgctgggagttgttttcctggcatcagtttctgtctcatttggc

SEQ ID NO: 714 - protein, 24 aa, unidentified
  REGION 1..24: note = sig12 signal peptide
MMRARFPLLLLGVVFLASVSVSFG

SEQ ID NO: 715 - DNA, 63 bp, unidentified
  misc_feature 1..63: note = coixss signal peptide encoding sequence
  source 1..63: mol_type = other DNA
atggctaccaagatatttgccctccttgtgctccttgctctttcagcgagcgctacaactgcg

SEQ ID NO: 716 - protein, 21 aa, unidentified
  REGION 1..21: note = coixss signal peptide
MATKIFALLVLLALSASATTA

SEQ ID NO: 717 - DNA, 12 bp, unidentified
  misc_feature 1..12: note = KDEL signal peptide encoding sequence
  source 1..12: mol_type = other DNA
aaggatgagctt

SEQ ID NO: 718 - DNA, 253 bp, unidentified
  misc_feature 1..253: note = nosT terminator
  source 1..253: mol_type = genomic DNA
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 719 - DNA, 480 bp, unidentified
  misc_feature 1..480: note = EU T terminator
  source 1..480: mol_type = genomic DNA
aaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgac

SEQ ID NO: 720 - DNA, 414 bp, unidentified
  misc_feature 1..414: note = StUbi3T terminator
  source 1..414: mol_type = genomic DNA
ctgattttaatgtttagcaaatgtcttatcagttttctctttttgtcgaacggtaatttagagttttttttgctatatggattttcgtttttgatgtatgtgacaaccctcgggattgttgatttatttcaaaactaagagtttttgtcttattgttctcgtctattttggaatatcaatcttagttttatatcttttctagttctctacgtgttaaatgttcaacacactagcaatttggcctgccagcgtatggattatggaactatcaagtgtgtgggatcgataaatatgcttctcaggaatttgagattttacagtctttatgctcattgggttgagtataatatagtaaaaaaatagtaaatttaagcaataatgttaggtgctatgtgtctgtcgagactattggcc

SEQ ID NO: 721 - DNA, 249 bp, unidentified
  misc_feature 1..249: note = AtHSP T terminator
  source 1..249: mol_type = genomic DNA
atatgaagatgaagatgaaatatttggtgtgtcaaataaaaagcttgtgtgcttaagtttgtgtttttttcttggcttgttgtgttatgaatttgtggctttttctaatatcaaatgaatgtaagatctcattataatgaataaacaaatgtttctataatccattgtgaatgttttgttggatctcttctgcagcatataactactgtatgtgctatggtatggactatggaatatgattaaagataa

SEQ ID NO: 722 - DNA, 376 bp, unidentified
  misc_feature 1..376: note = AtUbi10T terminator
  source 1..376: mol_type = genomic DNA
atctcgtctctgttatgcttaagaagttcaatgtttcgtttcatgtaaaactttggtggtttgtgttttggggccttgtataatccctgatgaataagtgttctactatgtttccgttcctgttatctctttctttctaatgacaagtcgaacttcttctttatcatcgcttcgtttttattatctgtgcttcttttgtttaatacgcctgcaaagtgactcgactctgtttagtgcagttctgcgaaacttgtaaatagtccaattgttggcctctagtaatagatgtagcgaaagtgttgagctgttgggttctaaggatggcttgaacatgttaatcttttaggttctgagtatgatgaacattcgttgttgc

SEQ ID NO: 723 - DNA, 916 bp, unidentified
  misc_feature 1..916: note = Rb7T terminator
  source 1..916: mol_type = genomic DNA
taaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagt

SEQ ID NO: 724 - DNA, 1190 bp, unidentified
  misc_feature 1..1190: note = TM6T terminator
  source 1..1190: mol_type = genomic DNA
gacatcctaggttcaatcaaattttactcgcatattgtagactttatccttttgtaattgttgcaaatttcttataaaattgattatctatattttaatcaaacatatatatacacttccaaataataaaatataatgacaacaaaacaatcaagcacaaaaaatgcctataacaaataaaaattacaacatacttttaccctgattcaaatcttcaaacactatgccagacaccataatccttctggatataggataaaaatttaaagtgattttttaccaattactatttcataaattgttcaaatacaaaatatgatattttaattattcccaactttttgagcctcctataactaatcaatataaaaaaataatttatcgattaagactaaagcaaaaaatattaccgatttgagttacaataaaaagttttatatcacgttatggtattgtgaattactctaacttcctagttcttgggttctagcttttcttggctctctgaatcttcaaaacctatatttgataaagccataacatacactaatgctcccatgcaaagtgcttctaaaactccttaacttggtctacggtaaaatttcttctaaaacaaaagcgactatcaacttctaatcgttgaacaaataattcatctccaataaaggattttaacaataaatatgaaataagaagtctatttctagttaaataaccaacaatatcccaaacatttatgaaatcaatatatgactgcattacaatttgatcccaaaatgcaaaaataaaattgcatctctattatagagtaaaaataatgcatcatcaattactaaccgattttactaacacgagaatctaattctcttccacaaagtaaaactcaatgtcaccgtcaattatttaagaatttgaattatattccaacaactgagtaagaaactatataattgtggggggagggggggccaaccctaaaagtttacttctcataaaaggctattagaaaggaaaggatacataaaaagaagagcaaagagagatcggagaagagagaaaaagtatatgaatttattagaagtacttttacttattagaggtaagagagttctagactgatttggataccatattagagttattaccgatataaaaatccttggttatgttaattaaatttctaaatatta

SEQ ID NO: 725 - DNA, 1197 bp, unidentified
  misc_feature 1..1197: note = arcT terminator
  source 1..1197: mol_type = genomic DNA
aataaataaaatgggagcaataaataaaatgggagctcatatatttacaccatttacactgtctattattcaccatgccaattattacttcataattttaaaattatgtcatttttaaaaattgcttaatgatggaaaggattattataagttaaaagtataacatagataaactaaccacaaaacaaatcaatataaactaacttactctcccatctaatttttatttaaatttctttacacttctcttccatttctatttctacaacattatttaacatttttattgtatttttcttactttctaactctattcatttcaaaaatcaatatatgtttatcaccacctctctaaaaaaaactttacaatcattggtccagaaaagttaaatcacgagatggtcattttagcattaaaacaacgattcttgtatcactatttttcagcatgtagtccattctcttcaaacaaagacagcggctatataatcgttgtgttatattcagtctaaaacaattgttatggtaaaagtcgtcattttacgcctttttaaaagatataaaatgacagttatggttaaaagtcatcatgttagatcctccttaaagatataaaatgacagttttggataaaaagtggtcattttatacgctcttgaaagatataaaacgacggttatggtaaaagctgccattttaaatgaaatatttttgttttagttcattttgtttaatgctaatcccatttaaattgacttgtacaattaaaactcacccacccagatacaatataaactaacttactctcacagctaagttttatttaaatttctttacacttcttttccatttctatttctatgacattaactaacatttttctcgtaattttttttcttattttctaactctatccatttcaaatcgatatatgtttatcaccaccactttaaaaagaaaatttacaatttctcgtgcaaaaaagctaaatcatgaccgtcattttagcattaaaacaacgattcttgtatcgttgtttttcagcatgtagtccattcttttcaagcaaagacaacagctatataatcatcgtgttatattcagtctaaaacaacagtaatgataaaagtcatcattttaggcctttctgaaatatatagaacgacattcatggtaaaaaatcgtcattttagatcc

SEQ ID NO: 726 - DNA, 65 bp, unidentified
  misc_feature 1..65: note = soybean glutamine synthase 5' UTR
  source 1..65: mol_type = genomic DNA
gaattctctaaaagagatctttttctgctctttgaagaaagaagggtctttgcttgattttggag

SEQ ID NO: 727 - DNA, 64 bp, unidentified
  misc_feature 1..64: note = OVAL 5' UTR
  source 1..64: mol_type = genomic DNA
acatacagctagaaagctgtattgcctttagcactcaagctcaaaagacaactcagagttcacc

SEQ ID NO: 728 - DNA, 65 bp, unidentified
  misc_feature 1..65: note = LG 5' UTR
  source 1..65: mol_type = genomic DNA
cccgagcccgctgtctcagccctccactccctgcagagctcagaagcgtgaccccagctgcagcc

SEQ ID NO: 729 - DNA, 13 bp, unidentified
  misc_feature 1..13: note = arcUTR 5' UTR
  source 1..13: mol_type = genomic DNA
tgaatgcatgatc

SEQ ID NO: 730 - DNA, 234 bp, unidentified
  misc_feature 1..234: note = Ubimonomer
  source 1..234: mol_type = other DNA
atgcagattttcgtgaagaccttaacggggaagacgatcaccctagaggttgagtcttccgacaccatcgacaatgtcaaagccaagatccaggacaaggaagggatacccccagaccagcagcgtttgattttcgccggaaagcagcttgaggatggtcgtactcttgccgactacaacatccagaaggagtcaactctccatctcgtgctccgtctccgtggtggtggttcc

SEQ ID NO: 731 - protein, 78 aa, unidentified
  REGION 1..78: note = Ubimonomer
MQIFVKTLTGKTITLEVESSDTIDNVKAKIQDKEGIPPDQQRLIFAGKQLEDGRTLADYNIQKESTLHLVLRLRGGGS

SEQ ID NO: 732 - DNA, 20 bp, synthetic construct
  misc_feature 1..20: note = Seed2 forward primer
  source 1..20: mol_type = other DNA
ctgagtttggatctctccgc

SEQ ID NO: 733 - DNA, 20 bp, synthetic construct
  misc_feature 1..20: note = Seed2 reverse primer
  source 1..20: mol_type = other DNA
acttgtatcaatgcccgtcc

SEQ ID NO: 734 - DNA, 20 bp, synthetic construct
  misc_feature 1..20: note = TUA5 forward primer
  source 1..20: mol_type = other DNA
ggatgtcaatgctgctgttg

SEQ ID NO: 735 - DNA, 20 bp, synthetic construct
  misc_feature 1..20: note = TUA5 reverse primer
  source 1..20: mol_type = other DNA
aaccttagcaaggtcaccac

SEQ ID NO: 736 - DNA, 20 bp, synthetic construct
  misc_feature 1..20: note = OLG1 forward primer
  source 1..20: mol_type = other DNA
acgaacaaggcattggcagg

SEQ ID NO: 737 - DNA, 20 bp, synthetic construct
  misc_feature 1..20: note = OLG1 reverse primer
  source 1..20: mol_type = other DNA
aggtgctcgtactggacact

SEQ ID NO: 738 - DNA, 21 bp, synthetic construct
  misc_feature 1..21: note = OLG2 forward primer
  source 1..21: mol_type = other DNA
tcgatgctctcaacgagaaca

SEQ ID NO: 739 - DNA, 22 bp, synthetic construct
  misc_feature 1..22: note = OLG2 reverse primer
  source 1..22: mol_type = other DNA
tcctcacaaggcattggcaaac

SEQ ID NO: 740 - DNA, 21 bp, synthetic construct
  misc_feature 1..21: note = OLG3 forward primer
  source 1..21: mol_type = other DNA
agaccaaaattcctgctgtgt

SEQ ID NO: 741 - DNA, 20 bp, synthetic construct
  misc_feature 1..20: note = OLG3 reverse primer
  source 1..20: mol_type = other DNA
tgattgttctggctctgcgc

SEQ ID NO: 742 - DNA, 22 bp, synthetic construct
  misc_feature 1..22: note = OLG4 forward primer
  source 1..22: mol_type = other DNA
ctggtcctcgacactgattata

SEQ ID NO: 743 - DNA, 21 bp, synthetic construct
  misc_feature 1..21: note = OLG4 reverse primer
  source 1..21: mol_type = other DNA
aagtgcctcgtcatcaacctc

SEQ ID NO: 744 - DNA, 20 bp, synthetic construct
  misc_feature 1..20: note = OOVAL1 forward primer
  source 1..20: mol_type = other DNA
gccgaggaaagataccccat

SEQ ID NO: 745 - DNA, 21 bp, synthetic construct
  misc_feature 1..21: note = OOVAL1 reverse primer
  source 1..21: mol_type = other DNA
tgtctggctctctacccaaga

SEQ ID NO: 746 - DNA, 20 bp, synthetic construct
  misc_feature 1..20: note = OOVAL2 forward primer
  source 1..20: mol_type = other DNA
aacgctaccctatcctgcca

SEQ ID NO: 747 - DNA, 21 bp, synthetic construct
  misc_feature 1..21: note = OOVAL2 reverse primer
  source 1..21: mol_type = other DNA
tggctctcgacccaagaattg

SEQ ID NO: 748 - DNA, 20 bp, synthetic construct
  misc_feature 1..20: note = OOVAL3 forward primer
  source 1..20: mol_type = other DNA
cagcagactgtacgcagaag

SEQ ID NO: 749 - DNA, 20 bp, synthetic construct
  misc_feature 1..20: note = OOVAL3 reverse primer
  source 1..20: mol_type = other DNA
tcagctcacgagcctgatct

SEQ ID NO: 750 - DNA, 20 bp, synthetic construct
  misc_feature 1..20: note = OOVAL4 forward primer
  source 1..20: mol_type = other DNA
aggagctgcgtctatggagt

SEQ ID NO: 751 - DNA, 21 bp, synthetic construct
  misc_feature 1..21: note = OOVAL4 reverse primer
  source 1..21: mol_type = other DNA
tgcacccaagtaaaccatggc

SEQ ID NO: 752 - DNA, 2625 bp, synthetic construct
  misc_feature 1..2625: note = Construct AR07-22
  source 1..2625: mol_type = other DNA
catcggtgattgattcctttaaagacttatgtttcttatcttgcttctgaggcaagtattcagttaccagttaccacttatattctggactttctgactgcatcctcatttttccaacattttaaatttcactattggctgaatgcttcttctttgaggaagaaacaattcagatggcagaaatgtatcaaccaatgcatatatacaaatgtacctcttgttctcaaaacatctatcggatggttccatttgctttgtcatccaattagtgactactttatattattcactcctctttattactattttcatgcgaggttgccatgtacattatatttgtaaggattgacgctattgagcgtttttcttcaattttctttattttagacatgggtatgaaatgtgtgttagagttgggttgaatgagatatacgttcaagtgaagtggcataccgttgtcgagtaaggatgacctacccattcttgagacaaatgttacattttagtatcagagtaaaatgtgtacctataactcaaattcgattgacatgtatccattcaacataaaattaaaccagcctgcacctgcatccacatttcaagtattttcaaaccgttcggctcctatccaccgggtgtaacaagacggattccgaatttggaagattttgactcaaattcccaatttatattgaccgtgactaaatcaactttaacttctataattctgattaagctcccaatttatattcccaacggcactacctccaaaatttatagactctcatccccttttaaaccaacttagtaaacgtttttttttttaattttatgaagttaagtttttaccttgtttttaaaaagaatcgttcataagatgccatgccagaacattagctacacgttacacatagcatgcagccgcggagaattgtttttcttcgccacttgtcactcccttcaaacacctaagagcttctctctcacagcacacacatacaatcacatgcgtgcatgcattattacacgtgatcgccatgcaaatctcctttatagcctataaattaactcatccgcttcactctttactcaaaccaaaactcatcaatacaaacaagattaaaaacataatgatgagagcacggttcccattactgttgctgggacttgttttcctggcttcagtttctgtctcaggtagcattggggctgcttctatggaattttgtttcgatgtctttaaagaacttaaggtacaccatgcaaatgagaacattttctactgtcccatcgctataatgtctgcacttgcaatggtttaccttggggctaaagacagtactcgtacacaaataaataaagtagtgagattcgataagttgcctgggttcggggattctatcgaagctcaatgtgggaccagtgttaacgtacatagctccttgcgcgatatcttgaatcaaataacaaagcctaatgatgtatactcattttcattggcctctcgcttgtatgccgaggaaagataccccattctgccagaataccttcagtgcgtcaaggaactctaccgcggaggactcgagcccataaatttccagactgcagcagaccaggccagggagctgattaactcttgggtagagagccagacaaatggcataatcaggaatgtgctgcagccatcatcagttgattcacaaacagctatggtgctggttaatgcaatcgtcttcaaagggttgtgggaaaaggcttttaaggacgaagatactcaagctatgcctttccgtgtaacagagcaagaaagcaagcctgtacaaatgatgtatcagattggtctgtttcgtgttgcctctatggcttcagagaaaatgaagatactcgaacttcccttcgcatcagggactatgagcatgttggttttgttgcctgatgaggtatctggtttggaacagctggaatcaataatcaatttcgagaagttgacagaatggaccagttctaatgttatggaagagcgtaagataaaagtatatttgcctcgtatgaaaatggaagaaaagtacaatttgaccagcgttttgatggctatgggcatcactgacgttttttcatcttctgctaatctcagcggcatatccagcgcagagagcctcaaaatatcccaagccgtccatgctgcacatgcagagataaatgaggctggtagggaagtggtcgggagcgctgaagctggggtagatgcagccagtgtaagtgaagagttcagggctgaccatcccttcctgttctgcattaagcacattgcaactaacgcagtactcttttttggacgttgcgtgagccccaaggatgagctttaaagatctgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 753 - DNA, 2870 bp, synthetic construct
  misc_feature 1..2870: note = Construct AR07-23
  source 1..2870: mol_type = other DNA
aacacaagcttcaagttttaaaaggaaaaatgtcagccaaaaactttaaataaaatggtaacaaggaaattattcaaaaattacaaacctcgtcaaaataggaaagaaaaaaagtttagggatttagaaaaaacatcaatctagttccaccttattttatagagagaagaaactaatatataagaactaaaaaacagaagaatagaaaaaaaaagtattgacaggaaagaaaaagtagctgtatgcttataagtactttgaggatttgaattctctcttataaaacacaaacacaatttttagattttatttaaataatcatcaatccgattataattatttatatatttttctattttcaaagaagtaaatcatgagcttttccaactcaacatctattttttttctctcaacctttttcacatcttaagtagtctcaccctttatatatataacttatttcttaccttttacattatgtaacttttatcaccaaaaccaacaactttaaaattttattaaatagactccacaagtaacttgacactcttacattcatcgacattaacttttatctgttttataaatattattgtgatataatttaatcaaaataaccacaaactttcataaaaggttcttattaagcatggcatttaataagcaaaaacaactcaatcactttcatataggaggtagcctaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttcacaactcaaacattctctccattggtccttaaacactcatcagtcatcaccatggccaagctagttttttccctttgttttctgcttttcagtggctgctgcttcgctggtagcattggggctgcttctatggaattttgtttcgatgtctttaaagaacttaaggtacaccatgcaaatgagaacattttctactgtcccatcgctataatgtctgcacttgcaatggtttaccttggggctaaagacagtactcgtacacaaataaataaagtagtgagattcgataagttgcctgggttcggggattctatcgaagctcaatgtgggaccagtgttaacgtacatagctccttgcgcgatatcttgaatcaaataacaaagcctaatgatgtatactcattttcattggcctctcgcttgtatgccgaggaaagataccccattctgccagaataccttcagtgcgtcaaggaactctaccgcggaggactcgagcccataaatttccagactgcagcagaccaggccagggagctgattaactcttgggtagagagccagacaaatggcataatcaggaatgtgctgcagccatcatcagttgattcacaaacagctatggtgctggttaatgcaatcgtcttcaaagggttgtgggaaaaggcttttaaggacgaagatactcaagctatgcctttccgtgtaacagagcaagaaagcaagcctgtacaaatgatgtatcagattggtctgtttcgtgttgcctctatggcttcagagaaaatgaagatactcgaacttcccttcgcatcagggactatgagcatgttggttttgttgcctgatgaggtatctggtttggaacagctggaatcaataatcaatttcgagaagttgacagaatggaccagttctaatgttatggaagagcgtaagataaaagtatatttgcctcgtatgaaaatggaagaaaagtacaatttgaccagcgttttgatggctatgggcatcactgacgttttttcatcttctgctaatctcagcggcatatccagcgcagagagcctcaaaatatcccaagccgtccatgctgcacatgcagagataaatgaggctggtagggaagtggtcgggagcgctgaagctggggtagatgcagccagtgtaagtgaagagttcagggctgaccatcccttcctgttctgcattaagcacattgcaactaacgcagtactcttttttggacgttgcgtgagccccaaggatgagctttaaagatctgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 754 - DNA, 2946 bp, synthetic construct
  misc_feature 1..2946: note = Construct AR07-25
  source 1..2946: mol_type = other DNA
caactattatcgcatgatgatgtacgttaagtcatcatcatctttaactttatatattgttaaaagtagaaaaaataggtgatgcattataaaataattttataacatcatttaattataaattatttataataaatatttgagtttttatagtaattacctaaacaattatatcaagactaatgcctgattagttgacatgacgaaattaaactcataaaagtaaagatgtttatgtggaaaactcttatacaattgagcggacttttttccatggtagttcagttttcttctattcaatttatttttttggtttccgctcagaataagaataatttgataaattcatttttaggcaattaagaatatttatttgactaactttttaattgaaataaatttacaataaatactcaatttatctttcacaatcaaaagattgagatgttgtaagatctccgataatatacttatatcttttcatttattacgttttcaaatttgaattttaatgtgtgttgtaagtataaatttaaaataaaaataaaaacaattattatatcaaaatggcaaaaacatttaatacgtattatttaagaaaaaaatatgtaataatatatttatattttaatatctattcttatgtattttttaaaaatctattatatattgatcaactaaaatatttttatatctacacttattttgcatttttatcaattttcttgcgttttttggcatatttaataatgactattctttaataatcaatcattattcttacatggtacatattgttggaaccatatgaagtgtccattgcatttgactatgtggatagtgttttgatccaggcctccatttgccgcttattaattaatttggtaacagtccgtactaatcagttacttatccttcctccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagcccacacatttgttgtttctaacccaacctcaaactcgtattctcttccgccacctcatttttgtttatttcaacacccgtcaaactgcatgccaccccgtggccaaatgtccatgcatgttaacaagacctatgactataaatatctgcaatctcggcccaggttttcatcatcaagaaccagttcaatatcctagtacaccgtattaaagaatttaagatatactatggctaccaagatatttgccctccttgtgctccttgctctttcagcgagcgctacaactgcgggtagcattggggctgcttctatggaattttgtttcgatgtctttaaagaacttaaggtacaccatgcaaatgagaacattttctactgtcccatcgctataatgtctgcacttgcaatggtttaccttggggctaaagacagtactcgtacacaaataaataaagtagtgagattcgataagttgcctgggttcggggattctatcgaagctcaatgtgggaccagtgttaacgtacatagctccttgcgcgatatcttgaatcaaataacaaagcctaatgatgtatactcattttcattggcctctcgcttgtatgccgaggaaagataccccattctgccagaataccttcagtgcgtcaaggaactctaccgcggaggactcgagcccataaatttccagactgcagcagaccaggccagggagctgattaactcttgggtagagagccagacaaatggcataatcaggaatgtgctgcagccatcatcagttgattcacaaacagctatggtgctggttaatgcaatcgtcttcaaagggttgtgggaaaaggcttttaaggacgaagatactcaagctatgcctttccgtgtaacagagcaagaaagcaagcctgtacaaatgatgtatcagattggtctgtttcgtgttgcctctatggcttcagagaaaatgaagatactcgaacttcccttcgcatcagggactatgagcatgttggttttgttgcctgatgaggtatctggtttggaacagctggaatcaataatcaatttcgagaagttgacagaatggaccagttctaatgttatggaagagcgtaagataaaagtatatttgcctcgtatgaaaatggaagaaaagtacaatttgaccagcgttttgatggctatgggcatcactgacgttttttcatcttctgctaatctcagcggcatatccagcgcagagagcctcaaaatatcccaagccgtccatgctgcacatgcagagataaatgaggctggtagggaagtggtcgggagcgctgaagctggggtagatgcagccagtgtaagtgaagagttcagggctgaccatcccttcctgttctgcattaagcacattgcaactaacgcagtactcttttttggacgttgcgtgagccccaaggatgagctttaaagatctgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 755 - DNA, 2955 bp, synthetic construct
  misc_feature 1..2955: note = Construct AR07-26
  source 1..2955: mol_type = other DNA
caactattatcgcatgatgatgtacgttaagtcatcatcatctttaactttatatattgttaaaagtagaaaaaataggtgatgcattataaaataattttataacatcatttaattataaattatttataataaatatttgagtttttatagtaattacctaaacaattatatcaagactaatgcctgattagttgacatgacgaaattaaactcataaaagtaaagatgtttatgtggaaaactcttatacaattgagcggacttttttccatggtagttcagttttcttctattcaatttatttttttggtttccgctcagaataagaataatttgataaattcatttttaggcaattaagaatatttatttgactaactttttaattgaaataaatttacaataaatactcaatttatctttcacaatcaaaagattgagatgttgtaagatctccgataatatacttatatcttttcatttattacgttttcaaatttgaattttaatgtgtgttgtaagtataaatttaaaataaaaataaaaacaattattatatcaaaatggcaaaaacatttaatacgtattatttaagaaaaaaatatgtaataatatatttatattttaatatctattcttatgtattttttaaaaatctattatatattgatcaactaaaatatttttatatctacacttattttgcatttttatcaattttcttgcgttttttggcatatttaataatgactattctttaataatcaatcattattcttacatggtacatattgttggaaccatatgaagtgtccattgcatttgactatgtggatagtgttttgatccaggcctccatttgccgcttattaattaatttggtaacagtccgtactaatcagttacttatccttcctccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagcccacacatttgttgtttctaacccaacctcaaactcgtattctcttccgccacctcatttttgtttatttcaacacccgtcaaactgcatgccaccccgtggccaaatgtccatgcatgttaacaagacctatgactataaatatctgcaatctcggcccaggttttcatcatcaagaaccagttcaatatcctagtacaccgtattaaagaatttaagatatactatgatgagagcgcggttcccattactgttgctgggagttgttttcctggcatcagtttctgtctcatttggcggtagcattggggctgcttctatggaattttgtttcgatgtctttaaagaacttaaggtacaccatgcaaatgagaacattttctactgtcccatcgctataatgtctgcacttgcaatggtttaccttggggctaaagacagtactcgtacacaaataaataaagtagtgagattcgataagttgcctgggttcggggattctatcgaagctcaatgtgggaccagtgttaacgtacatagctccttgcgcgatatcttgaatcaaataacaaagcctaatgatgtatactcattttcattggcctctcgcttgtatgccgaggaaagataccccattctgccagaataccttcagtgcgtcaaggaactctaccgcggaggactcgagcccataaatttccagactgcagcagaccaggccagggagctgattaactcttgggtagagagccagacaaatggcataatcaggaatgtgctgcagccatcatcagttgattcacaaacagctatggtgctggttaatgcaatcgtcttcaaagggttgtgggaaaaggcttttaaggacgaagatactcaagctatgcctttccgtgtaacagagcaagaaagcaagcctgtacaaatgatgtatcagattggtctgtttcgtgttgcctctatggcttcagagaaaatgaagatactcgaacttcccttcgcatcagggactatgagcatgttggttttgttgcctgatgaggtatctggtttggaacagctggaatcaataatcaatttcgagaagttgacagaatggaccagttctaatgttatggaagagcgtaagataaaagtatatttgcctcgtatgaaaatggaagaaaagtacaatttgaccagcgttttgatggctatgggcatcactgacgttttttcatcttctgctaatctcagcggcatatccagcgcagagagcctcaaaatatcccaagccgtccatgctgcacatgcagagataaatgaggctggtagggaagtggtcgggagcgctgaagctggggtagatgcagccagtgtaagtgaagagttcagggctgaccatcccttcctgttctgcattaagcacattgcaactaacgcagtactcttttttggacgttgcgtgagccccaaggatgagctttaaagatctgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 756 - DNA, 4019 bp, synthetic construct
  misc_feature 1..4019: note = Construct AR07-27
  source 1..4019: mol_type = other DNA
cattgtactcccagtatcattatagtgaaagttttggctctctcgccggtggttttttacctctatttaaaggggttttccacctaaaaattctggtatcattctcactttacttgttactttaatttctcataatctttggttgaaattatcacgcttccgcacacgatatccctacaaatttattatttgttaaacattttcaaaccgcataaaattttatgaagtcccgtctatctttaatgtagtctaacattttcatattgaaatatataatttacttaattttagcgttggtagaaagcataatgatttattcttattcttcttcatataaatgtttaatatacaatataaacaaattctttaccttaagaaggatttcccattttatattttaaaaatatatttatcaaatatttttcaaccacgtaaatcacataataataagttgtttcaaaagtaataaaatttaactccataatttttttatttgactgatcttaaagcaacacccagtgacacaactagccatttttttctttgaataaaaaaatccaattatcattgtattttttttatacaatgaaaatttcaccaaacaatgatttgtggtatttctgaagcaagtcatgttatgcaaaattctataattcccatttgacactacggaagtaactgaagatctgcttttacatgcgagacacatcttctaaagtaattttaataatagttactatattcaagatttcatatatcaaatactcaatattacttctaaaaaattaattagatataattaaaatattacttttttaattttaagtttaattgttgaatttgtgactattgatttattattctactatgtttaaattgttttataggtagtttaaagtaaatataagtaatgtagtagagtgttagagtgttaccctaaaccataaactataagatttatggtggactaattttcatatatttcttattgcttttaccttttcttggtatgtaagtccgtaactggaattactgtgggttgccatgacactctgtggtcttttggttcatgcatggatgcttgcgcaagaaaaagacaaagaacaaagaaaaaagacaaaacagagagacaaaacgcaatcacacaaccaactcaaattagtcactggctgatcaagatcgccgcgtccatgtatgtctaaatgccatgcaaagcaacacgtgcttaacatgcactttaaatggctcacccatcccaacccactcacaaacacattgcctttttcttcatcatcaccacaaccacctgtatatattcattctcttccgccacctcaatttcttcacttcaacacacgtcaacctgcatatgcgtgtcatcccatgcccaaatctccatgcatgttcctaccaccttctctcttatataatacctataaatacctctaatatcactcacttctttcatcatccatccatccagagtactactactctactactataataccccaacccaactcatattcaatactactctacttgaatgcatgatcatggctacttcaaagttgaaaacccagaatgtggttgtatctctctccctaaccttaaccttggtactggtgctactgaccagcaaggcaaactcaggtagcattggggctgcttctatggaattttgtttcgatgtctttaaagaacttaaggtacaccatgcaaatgagaacattttctactgtcccatcgctataatgtctgcacttgcaatggtttaccttggggctaaagacagtactcgtacacaaataaataaagtagtgagattcgataagttgcctgggttcggggattctatcgaagctcaatgtgggaccagtgttaacgtacatagctccttgcgcgatatcttgaatcaaataacaaagcctaatgatgtatactcattttcattggcctctcgcttgtatgccgaggaaagataccccattctgccagaataccttcagtgcgtcaaggaactctaccgcggaggactcgagcccataaatttccagactgcagcagaccaggccagggagctgattaactcttgggtagagagccagacaaatggcataatcaggaatgtgctgcagccatcatcagttgattcacaaacagctatggtgctggttaatgcaatcgtcttcaaagggttgtgggaaaaggcttttaaggacgaagatactcaagctatgcctttccgtgtaacagagcaagaaagcaagcctgtacaaatgatgtatcagattggtctgtttcgtgttgcctctatggcttcagagaaaatgaagatactcgaacttcccttcgcatcagggactatgagcatgttggttttgttgcctgatgaggtatctggtttggaacagctggaatcaataatcaatttcgagaagttgacagaatggaccagttctaatgttatggaagagcgtaagataaaagtatatttgcctcgtatgaaaatggaagaaaagtacaatttgaccagcgttttgatggctatgggcatcactgacgttttttcatcttctgctaatctcagcggcatatccagcgcagagagcctcaaaatatcccaagccgtccatgctgcacatgcagagataaatgaggctggtagggaagtggtcgggagcgctgaagctggggtagatgcagccagtgtaagtgaagagttcagggctgaccatcccttcctgttctgcattaagcacattgcaactaacgcagtactcttttttggacgttgcgtgagccccaaggatgagctttaaaataaataaaatgggagcaataaataaaatgggagctcatatatttacaccatttacactgtctattattcaccatgccaattattacttcataattttaaaattatgtcatttttaaaaattgcttaatgatggaaaggattattataagttaaaagtataacatagataaactaaccacaaaacaaatcaatataaactaacttactctcccatctaatttttatttaaatttctttacacttctcttccatttctatttctacaacattatttaacatttttattgtatttttcttactttctaactctattcatttcaaaaatcaatatatgtttatcaccacctctctaaaaaaaactttacaatcattggtccagaaaagttaaatcacgagatggtcattttagcattaaaacaacgattcttgtatcactatttttcagcatgtagtccattctcttcaaacaaagacagcggctatataatcgttgtgttatattcagtctaaaacaattgttatggtaaaagtcgtcattttacgcctttttaaaagatataaaatgacagttatggttaaaagtcatcatgttagatcctccttaaagatataaaatgacagttttggataaaaagtggtcattttatacgctcttgaaagatataaaacgacggttatggtaaaagctgccattttaaatgaaatatttttgttttagttcattttgtttaatgctaatcccatttaaattgacttgtacaattaaaactcacccacccagatacaatataaactaacttactctcacagctaagttttatttaaatttctttacacttcttttccatttctatttctatgacattaactaacatttttctcgtaattttttttcttattttctaactctatccatttcaaatcgatatatgtttatcaccaccactttaaaaagaaaatttacaatttctcgtgcaaaaaagctaaatcatgaccgtcattttagcattaaaacaacgattcttgtatcgttgtttttcagcatgtagtccattcttttcaagcaaagacaacagctatataatcatcgtgttatattcagtctaaaacaacagtaatgataaaagtcatcattttaggcctttctgaaatatatagaacgacattcatggtaaaaaatcgtcattttagatcc

SEQ ID NO: 757 - DNA, 4259 bp, synthetic construct
  misc_feature 1..4259: note = Construct AR15-16
  source 1..4259: mol_type = other DNA
aacacaagcttcaagttttaaaaggaaaaatgtcagccaaaaactttaaataaaatggtaacaaggaaattattcaaaaattacaaacctcgtcaaaataggaaagaaaaaaagtttagggatttagaaaaaacatcaatctagttccaccttattttatagagagaagaaactaatatataagaactaaaaaacagaagaatagaaaaaaaaagtattgacaggaaagaaaaagtagctgtatgcttataagtactttgaggatttgaattctctcttataaaacacaaacacaatttttagattttatttaaataatcatcaatccgattataattatttatatatttttctattttcaaagaagtaaatcatgagcttttccaactcaacatctattttttttctctcaacctttttcacatcttaagtagtctcaccctttatatatataacttatttcttaccttttacattatgtaacttttatcaccaaaaccaacaactttaaaattttattaaatagactccacaagtaacttgacactcttacattcatcgacattaacttttatctgttttataaatattattgtgatataatttaatcaaaataaccacaaactttcataaaaggttcttattaagcatggcatttaataagcaaaaacaactcaatcactttcatataggaggtagcctaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttcacaactcaaacattctctccattggtccttaaacactcatcagtcatcaccatggccaagctagttttttccctttgttttctgcttttcagtggctgctgcttcgctggatcaattggcgccgcatctatggagttctgcttcgatgtttttaaagagcttaaagtgcaccatgccaacgagaatatcttctattgcccaattgccattatgtctgcccttgctatggtgtacttgggtgctaaagactctactaggacccagataaacaaggtagtcagattcgacaagctgcctgggtttggcgactctattgaagctcagtgtggtacttctgttaatgtccactcatccctccgcgacatacttaatcaaattacaaaaccaaatgatgtgtactcatttagtctggccagccgtttgtacgcagaggaacgctaccctatcctgccagagtatttgcaatgtgtgaaggaactttacaggggtgggcttgagccaataaactttcaaacagcagccgaccaagctagggagcttatcaattcttgggtcgagagccaaactaacggaatcatccgcaacgtcctccagccaagttccgttgattcccagaccgctatggtacttgtgaatgccattgtcttcaaggggctttgggagaaggcatttaaagacgaggacactcaggcaatgccctttcgtgtgaccgagcaggagtcaaaacctgttcaaatgatgtaccaaattgggctgttcagagttgctagtatggcctctgagaaaatgaagatccttgaactcccatttgcctccgggacaatgtctatgcttgtcctcctgccagatgaagtcagtgggctcgaacagctcgaaagcataataaactttgagaaacttaccgaatggacttcttccaatgttatggaggagcgtaaaattaaggtctatctgccccgcatgaaaatggaggaaaagtataatctcactagcgtcctcatggctatgggaattactgatgtattctcctctagcgctaatctgagtggaatctccagcgccgagtctctcaagataagccaggccgtgcacgctgctcatgctgaaatcaacgaagccggcagagaggtggtggggtcagctgaggcaggtgtagatgcagccagtgtctctgaggaatttagagccgatcaccctttccttttttgcattaaacatatcgctacaaatgccgttttgtttttcggtcgttgcgttagtccaaaggatgagctttaaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgactcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagt

SEQ ID NO: 758 - DNA, 4259 bp, synthetic construct
  misc_feature 1..4259: note = Construct AR15-17
  source 1..4259: mol_type = other DNA
aacacaagcttcaagttttaaaaggaaaaatgtcagccaaaaactttaaataaaatggtaacaaggaaattattcaaaaattacaaacctcgtcaaaataggaaagaaaaaaagtttagggatttagaaaaaacatcaatctagttccaccttattttatagagagaagaaactaatatataagaactaaaaaacagaagaatagaaaaaaaaagtattgacaggaaagaaaaagtagctgtatgcttataagtactttgaggatttgaattctctcttataaaacacaaacacaatttttagattttatttaaataatcatcaatccgattataattatttatatatttttctattttcaaagaagtaaatcatgagcttttccaactcaacatctattttttttctctcaacctttttcacatcttaagtagtctcaccctttatatatataacttatttcttaccttttacattatgtaacttttatcaccaaaaccaacaactttaaaattttattaaatagactccacaagtaacttgacactcttacattcatcgacattaacttttatctgttttataaatattattgtgatataatttaatcaaaataaccacaaactttcataaaaggttcttattaagcatggcatttaataagcaaaaacaactcaatcactttcatataggaggtagcctaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttcacaactcaaacattctctccattggtccttaaacactcatcagtcatcaccatggccaagctagttttttccctttgttttctgcttttcagtggctgctgcttcgctgggtccatcggtgccgcctcaatggaattctgctttgacgtcttcaaggaacttaaggtacatcatgccaacgagaatattttttactgtccaatagctatcatgagtgcacttgctatggtgtaccttggagccaaagactcaacccgtacccagatcaacaaggtggtccgctttgacaaactgccagggtttggggattctattgaggcccaatgcggaacaagtgtgaacgtccactctagcttgcgcgatatacttaatcaaataactaaaccaaatgatgtgtattcattctctctcgccagcagactgtacgcagaagaaaggtatcccattctccccgagtacctccaatgcgtaaaggagttgtacagaggcggcctggaacccataaatttccaaactgccgcagatcaggctcgtgagctgataaattcatgggtcgagagccaaactaacggtatcattcgtaatgtccttcaaccctcaagtgtggacagtcagacagccatggtcctcgtcaatgctatagtcttcaaaggcctgtgggaaaagacctttaaggatgaagatactcaagcaatgccctttagagtcacagagcaagaaagcaaacccgtgcaaatgatgtatcaaatcgggctctttcgtgttgcatccatggcatctgaaaagatgaagatattggaactccccttcgcctctggaacaatgagtatgttggtacttctgcccgatgaggtctctgggttggaacagcttgaatctattattaacttcgagaaactgaccgagtggactagtagtaatgtcatggaggagagaaagattaaggtttatttgccacgcatgaagatggaagagaaatataacttgacatctgtactgatggcaatgggtataaccgacgtatttagcagtagcgccaatctgtcagggatttcttcagccgaaagtctcaagatttctcaggcagttcacgcagcccatgcagagataaacgaagcaggccgcgaagttgtcggatctgcagaagccggcgtggatgcagccagtgtctccgaagagttcagagcagaccaccctttcctcttctgcattaagcacatcgcaaccaacgcagtactttttttcggacgttgcgtgtccccaaaggatgagctttaaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgactcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagt

SEQ ID NO: 759 - DNA, 4259 bp, synthetic construct
  misc_feature 1..4259: note = Construct AR15-18
  source 1..4259: mol_type = other DNA
aacacaagcttcaagttttaaaaggaaaaatgtcagccaaaaactttaaataaaatggtaacaaggaaattattcaaaaattacaaacctcgtcaaaataggaaagaaaaaaagtttagggatttagaaaaaacatcaatctagttccaccttattttatagagagaagaaactaatatataagaactaaaaaacagaagaatagaaaaaaaaagtattgacaggaaagaaaaagtagctgtatgcttataagtactttgaggatttgaattctctcttataaaacacaaacacaatttttagattttatttaaataatcatcaatccgattataattatttatatatttttctattttcaaagaagtaaatcatgagcttttccaactcaacatctattttttttctctcaacctttttcacatcttaagtagtctcaccctttatatatataacttatttcttaccttttacattatgtaacttttatcaccaaaaccaacaactttaaaattttattaaatagactccacaagtaacttgacactcttacattcatcgacattaacttttatctgttttataaatattattgtgatataatttaatcaaaataaccacaaactttcataaaaggttcttattaagcatggcatttaataagcaaaaacaactcaatcactttcatataggaggtagcctaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttcacaactcaaacattctctccattggtccttaaacactcatcagtcatcaccatggccaagctagttttttccctttgttttctgcttttcagtggctgctgcttcgctggttcaataggagctgcgtctatggagttttgttttgatgtctttaaggaactcaaagtccaccacgccaatgaaaatattttctattgccctattgcaatcatgagtgcgctagccatggtttacttgggtgcaaaagacagtacgcgtactcaaataaacaaggttgttcgctttgacaagcttcctggatttggagatagtattgaagcacaatgtggaactagcgtaaacgtccacagctcattgagggacattcttaaccaaattaccaagccaaatgatgtatatagtttttccttggcatcacgactgtatgcagaagaaagatatcctatcctcccggaatatcttcagtgcgtgaaagaattatacagaggtgggctagagccaatcaattttcaaaccgctgctgatcaagctcgcgagttgattaactcatgggttgagagccagacaaatgggataataagaaatgttcttcaaccatctagtgtggactctcaaacagcaatggtgctcgtcaatgcgatagtttttaaaggcttgtgggagaaaacattcaaagatgaggatactcaggcaatgccattccgtgtaactgaacaggaatctaagcctgttcaaatgatgtatcagattggtttgttcagagttgcctctatggcatctgaaaaaatgaaaattttggagcttccatttgctagtggaacaatgtcaatgttagttttactgcctgatgaagtgtccggtttagaacaattggaatcaattatcaactttgaaaagttgaccgagtggacttcctccaatgtgatggaggagaggaagattaaggtgtaccttcccagaatgaagatggaagagaaatataacctgacttcggtcctaatggctatggggatcacagatgtgttttcttcctcggcaaacctttcaggcatatcaagcgccgagtcattgaaaatttcacaggctgttcatgcagctcatgctgaaatcaatgaggccgggcgggaggttgtgggcagtgctgaagctggagttgatgctgcctcagtgtctgaggaatttagagcagatcatcctttcctcttctgcattaagcatattgctaccaatgctgtactgttcttcggtaggtgtgttagccccaaggatgagctttaaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgactcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagt

SEQ ID NO: 760 - DNA, 4247 bp, synthetic construct
  misc_feature 1..4247: note = Construct AR15-19
  source 1..4247: mol_type = other DNA
aacacaagcttcaagttttaaaaggaaaaatgtcagccaaaaactttaaataaaatggtaacaaggaaattattcaaaaattacaaacctcgtcaaaataggaaagaaaaaaagtttagggatttagaaaaaacatcaatctagttccaccttattttatagagagaagaaactaatatataagaactaaaaaacagaagaatagaaaaaaaaagtattgacaggaaagaaaaagtagctgtatgcttataagtactttgaggatttgaattctctcttataaaacacaaacacaatttttagattttatttaaataatcatcaatccgattataattatttatatatttttctattttcaaagaagtaaatcatgagcttttccaactcaacatctattttttttctctcaacctttttcacatcttaagtagtctcaccctttatatatataacttatttcttaccttttacattatgtaacttttatcaccaaaaccaacaactttaaaattttattaaatagactccacaagtaacttgacactcttacattcatcgacattaacttttatctgttttataaatattattgtgatataatttaatcaaaataaccacaaactttcataaaaggttcttattaagcatggcatttaataagcaaaaacaactcaatcactttcatataggaggtagcctaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttcacaactcaaacattctctccattggtccttaaacactcatcagtcatcaccatggccaagctagttttttccctttgttttctgcttttcagtggctgctgcttcgctggatcaattggcgccgcatctatggagttctgcttcgatgtttttaaagagcttaaagtgcaccatgccaacgagaatatcttctattgcccaattgccattatgtctgcccttgctatggtgtacttgggtgctaaagactctactaggacccagataaacaaggtagtcagattcgacaagctgcctgggtttggcgactctattgaagctcagtgtggtacttctgttaatgtccactcatccctccgcgacatacttaatcaaattacaaaaccaaatgatgtgtactcatttagtctggccagccgtttgtacgcagaggaacgctaccctatcctgccagagtatttgcaatgtgtgaaggaactttacaggggtgggcttgagccaataaactttcaaacagcagccgaccaagctagggagcttatcaattcttgggtcgagagccaaactaacggaatcatccgcaacgtcctccagccaagttccgttgattcccagaccgctatggtacttgtgaatgccattgtcttcaaggggctttgggagaaggcatttaaagacgaggacactcaggcaatgccctttcgtgtgaccgagcaggagtcaaaacctgttcaaatgatgtaccaaattgggctgttcagagttgctagtatggcctctgagaaaatgaagatccttgaactcccatttgcctccgggacaatgtctatgcttgtcctcctgccagatgaagtcagtgggctcgaacagctcgaaagcataataaactttgagaaacttaccgaatggacttcttccaatgttatggaggagcgtaaaattaaggtctatctgccccgcatgaaaatggaggaaaagtataatctcactagcgtcctcatggctatgggaattactgatgtattctcctctagcgctaatctgagtggaatctccagcgccgagtctctcaagataagccaggccgtgcacgctgctcatgctgaaatcaacgaagccggcagagaggtggtggggtcagctgaggcaggtgtagatgcagccagtgtctctgaggaatttagagccgatcaccctttccttttttgcattaaacatatcgctacaaatgccgttttgtttttcggtcgttgcgttagtccataaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgactcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagt

SEQ ID NO: 761 - DNA, 5040 bp, synthetic construct
  misc_feature 1..5040: note = Construct AR15-20
  source 1..5040: mol_type = other DNA
aacacaagcttcaagttttaaaaggaaaaatgtcagccaaaaactttaaataaaatggtaacaaggaaattattcaaaaattacaaacctcgtcaaaataggaaagaaaaaaagtttagggatttagaaaaaacatcaatctagttccaccttattttatagagagaagaaactaatatataagaactaaaaaacagaagaatagaaaaaaaaagtattgacaggaaagaaaaagtagctgtatgcttataagtactttgaggatttgaattctctcttataaaacacaaacacaatttttagattttatttaaataatcatcaatccgattataattatttatatatttttctattttcaaagaagtaaatcatgagcttttccaactcaacatctattttttttctctcaacctttttcacatcttaagtagtctcaccctttatatatataacttatttcttaccttttacattatgtaacttttatcaccaaaaccaacaactttaaaattttattaaatagactccacaagtaacttgacactcttacattcatcgacattaacttttatctgttttataaatattattgtgatataatttaatcaaaataaccacaaactttcataaaaggttcttattaagcatggcatttaataagcaaaaacaactcaatcactttcatataggaggtagcctaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttcacaactcaaacattctctccattggtccttaaacactcatcagtcatcaccgttcgttatctaccaccgttctatggattttattccttctattcgtgtttattctattggtttatgttgcttgcaatatgttttttctgaatctgtcgtcgttgtcttcaattttatccatgtttcagagatcaattttgtttgtgtagtatgtgcttattcttcttcttttcgttcgagttgttaataacggtgctatggtgttttcaaaagtgttttttttattacttttgatttaaagtttttttggtaaggcttttatttgcttgttatattcaaatctttggatccagatcttatataagtttttggttcaagaaagtttttggttactgatgaatagatctattaactgttactttaatcgattcaagctaaagttttttggttactgatgaatagatctattatctgttacttttaatcggttcaagctcaagttttttggttactgatgaatagatctatatacgtcacagtgtgctaaacatgcccttgttttatctcgatcttatgtatgggagtgccataaattttgttatgtctatttttttatctgttggaatcatactgagtttgatgcgttacgattgagcatacctatttttgggcttgttgtatggtgggtatttagatcttaatctttttatgcttatgaaaggttttgtaatgacaaaggtcttaatgttgttaaacttttatttttactttatatggtgtgttgatgtgttatggttttgacaactttttttttttctggatttttgcagatttaaggaccatggccaagctagttttttccctttgttttctgcttttcagtggctgctgcttcgctggatcaattggcgccgcatctatggagttctgcttcgatgtttttaaagagcttaaagtgcaccatgccaacgagaatatcttctattgcccaattgccattatgtctgcccttgctatggtgtacttgggtgctaaagactctactaggacccagataaacaaggtagtcagattcgacaagctgcctgggtttggcgactctattgaagctcagtgtggtacttctgttaatgtccactcatccctccgcgacatacttaatcaaattacaaaaccaaatgatgtgtactcatttagtctggccagccgtttgtacgcagaggaacgctaccctatcctgccagagtatttgcaatgtgtgaaggaactttacaggggtgggcttgagccaataaactttcaaacagcagccgaccaagctagggagcttatcaattcttgggtcgagagccaaactaacggaatcatccgcaacgtcctccagccaagttccgttgattcccagaccgctatggtacttgtgaatgccattgtcttcaaggggctttgggagaaggcatttaaagacgaggacactcaggcaatgccctttcgtgtgaccgagcaggagtcaaaacctgttcaaatgatgtaccaaattgggctgttcagagttgctagtatggcctctgagaaaatgaagatccttgaactcccatttgcctccgggacaatgtctatgcttgtcctcctgccagatgaagtcagtgggctcgaacagctcgaaagcataataaactttgagaaacttaccgaatggacttcttccaatgttatggaggagcgtaaaattaaggtctatctgccccgcatgaaaatggaggaaaagtataatctcactagcgtcctcatggctatgggaattactgatgtattctcctctagcgctaatctgagtggaatctccagcgccgagtctctcaagataagccaggccgtgcacgctgctcatgctgaaatcaacgaagccggcagagaggtggtggggtcagctgaggcaggtgtagatgcagccagtgtctctgaggaatttagagccgatcaccctttccttttttgcattaaacatatcgctacaaatgccgttttgtttttcggtcgttgcgttagtccaaaggatgagctttaaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgactcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagt

SEQ ID NO: 762 - DNA, 5029 bp, synthetic construct
  misc_feature 1..5029: note = Construct AR15-21
  source 1..5029: mol_type = other DNA
aacacaagcttcaagttttaaaaggaaaaatgtcagccaaaaactttaaataaaatggtaacaaggaaattattcaaaaattacaaacctcgtcaaaataggaaagaaaaaaagtttagggatttagaaaaaacatcaatctagttccaccttattttatagagagaagaaactaatatataagaactaaaaaacagaagaatagaaaaaaaaagtattgacaggaaagaaaaagtagctgtatgcttataagtactttgaggatttgaattctctcttataaaacacaaacacaatttttagattttatttaaataatcatcaatccgattataattatttatatatttttctattttcaaagaagtaaatcatgagcttttccaactcaacatctattttttttctctcaacctttttcacatcttaagtagtctcaccctttatatatataacttatttcttaccttttacattatgtaacttttatcaccaaaaccaacaactttaaaattttattaaatagactccacaagtaacttgacactcttacattcatcgacattaacttttatctgttttataaatattattgtgatataatttaatcaaaataaccacaaactttcataaaaggttcttattaagcatggcatttaataagcaaaaacaactcaatcactttcatataggaggtagcctaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttcacaactcaaacattctctccattggtccttaaacactcatcagtcatcaccatggccaagctagttttttccctttgttttctgcttttcagtggctgctgcttcgctggatcaattggcgccgcatctatggagttctgcttcgatgtttttaaagagcttaaagtgcaccatgccaacgagaatatcttctattgcccaattgccattatgtctgcccttgctatggtgtacttgggtgctaaagactctactaggacccagataaacaaggttcgttatctaccaccgttctatggattttattccttctattcgtgtttattctattggtttatgttgcttgcaatatgttttttctgaatctgtcgtcgttgtcttcaattttatccatgtttcagagatcaattttgtttgtgtagtatgtgcttattcttcttcttttcgttcgagttgttaataacggtgctatggtgttttcaaaagtgttttttttattacttttgatttaaagtttttttggtaaggcttttatttgcttgttatattcaaatctttggatccagatcttatataagtttttggttcaagaaagtttttggttactgatgaatagatctattaactgttactttaatcgattcaagctaaagttttttggttactgatgaatagatctattatctgttacttttaatcggttcaagctcaagttttttggttactgatgaatagatctatatacgtcacagtgtgctaaacatgcccttgttttatctcgatcttatgtatgggagtgccataaattttgttatgtctatttttttatctgttggaatcatactgagtttgatgcgttacgattgagcatacctatttttgggcttgttgtatggtgggtatttagatcttaatctttttatgcttatgaaaggttttgtaatgacaaaggtcttaatgttgttaaacttttatttttactttatatggtgtgttgatgtgttatggttttgacaactttttttttttctggatttttgcaggtagtcagattcgacaagctgcctgggtttggcgactctattgaagctcagtgtggtacttctgttaatgtccactcatccctccgcgacatacttaatcaaattacaaaaccaaatgatgtgtactcatttagtctggccagccgtttgtacgcagaggaacgctaccctatcctgccagagtatttgcaatgtgtgaaggaactttacaggggtgggcttgagccaataaactttcaaacagcagccgaccaagctagggagcttatcaattcttgggtcgagagccaaactaacggaatcatccgcaacgtcctccagccaagttccgttgattcccagaccgctatggtacttgtgaatgccattgtcttcaaggggctttgggagaaggcatttaaagacgaggacactcaggcaatgccctttcgtgtgaccgagcaggagtcaaaacctgttcaaatgatgtaccaaattgggctgttcagagttgctagtatggcctctgagaaaatgaagatccttgaactcccatttgcctccgggacaatgtctatgcttgtcctcctgccagatgaagtcagtgggctcgaacagctcgaaagcataataaactttgagaaacttaccgaatggacttcttccaatgttatggaggagcgtaaaattaaggtctatctgccccgcatgaaaatggaggaaaagtataatctcactagcgtcctcatggctatgggaattactgatgtattctcctctagcgctaatctgagtggaatctccagcgccgagtctctcaagataagccaggccgtgcacgctgctcatgctgaaatcaacgaagccggcagagaggtggtggggtcagctgaggcaggtgtagatgcagccagtgtctctgaggaatttagagccgatcaccctttccttttttgcattaaacatatcgctacaaatgccgttttgtttttcggtcgttgcgttagtccaaaggatgagctttaaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgactcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagt

SEQ ID NO: 763 - DNA, 4352 bp, synthetic construct
  misc_feature 1..4352: note = Construct AR15-22
  source 1..4352: mol_type = other DNA
aacacaagcttcaagttttaaaaggaaaaatgtcagccaaaaactttaaataaaatggtaacaaggaaattattcaaaaattacaaacctcgtcaaaataggaaagaaaaaaagtttagggatttagaaaaaacatcaatctagttccaccttattttatagagagaagaaactaatatataagaactaaaaaacagaagaatagaaaaaaaaagtattgacaggaaagaaaaagtagctgtatgcttataagtactttgaggatttgaattctctcttataaaacacaaacacaatttttagattttatttaaataatcatcaatccgattataattatttatatatttttctattttcaaagaagtaaatcatgagcttttccaactcaacatctattttttttctctcaacctttttcacatcttaagtagtctcaccctttatatatataacttatttcttaccttttacattatgtaacttttatcaccaaaaccaacaactttaaaattttattaaatagactccacaagtaacttgacactcttacattcatcgacattaacttttatctgttttataaatattattgtgatataatttaatcaaaataaccacaaactttcataaaaggttcttattaagcatggcatttaataagcaaaaacaactcaatcactttcatataggaggtagcctaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttcacaactcaaacattctctccattggtccttaaacactcatcagtcatcaccatggccaagctagttttttccctttgttttctgcttttcagtggctgctgcttcgctggatcaattggcgccgcatctatggagttctgcttcgatgtttttaaagagcttaaagtgcaccatgccaacgagaatatcttctattgcccaattgccattatgtctgcccttgctatggtgtacttgggtgctaaagactctactaggacccagataaacaaggtaaccatatctttcatctgttatgtgactacacattgcttctctttttgtgttctgtctcattaattgcggtttgttacatgttgtttgtaggtagtcagattcgacaagctgcctgggtttggcgactctattgaagctcagtgtggtacttctgttaatgtccactcatccctccgcgacatacttaatcaaattacaaaaccaaatgatgtgtactcatttagtctggccagccgtttgtacgcagaggaacgctaccctatcctgccagagtatttgcaatgtgtgaaggaactttacaggggtgggcttgagccaataaactttcaaacagcagccgaccaagctagggagcttatcaattcttgggtcgagagccaaactaacggaatcatccgcaacgtcctccagccaagttccgttgattcccagaccgctatggtacttgtgaatgccattgtcttcaaggggctttgggagaaggcatttaaagacgaggacactcaggcaatgccctttcgtgtgaccgagcaggagtcaaaacctgttcaaatgatgtaccaaattgggctgttcagagttgctagtatggcctctgagaaaatgaagatccttgaactcccatttgcctccgggacaatgtctatgcttgtcctcctgccagatgaagtcagtgggctcgaacagctcgaaagcataataaactttgagaaacttaccgaatggacttcttccaatgttatggaggagcgtaaaattaaggtctatctgccccgcatgaaaatggaggaaaagtataatctcactagcgtcctcatggctatgggaattactgatgtattctcctctagcgctaatctgagtggaatctccagcgccgagtctctcaagataagccaggccgtgcacgctgctcatgctgaaatcaacgaagccggcagagaggtggtggggtcagctgaggcaggtgtagatgcagccagtgtctctgaggaatttagagccgatcaccctttccttttttgcattaaacatatcgctacaaatgccgttttgtttttcggtcgttgcgttagtccaaaggatgagctttaaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgactcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagt

SEQ ID NO: 764 - DNA, 4323 bp, synthetic construct
  misc_feature 1..4323: note = Construct AR15-23
  source 1..4323: mol_type = other DNA
aacacaagcttcaagttttaaaaggaaaaatgtcagccaaaaactttaaataaaatggtaacaaggaaattattcaaaaattacaaacctcgtcaaaataggaaagaaaaaaagtttagggatttagaaaaaacatcaatctagttccaccttattttatagagagaagaaactaatatataagaactaaaaaacagaagaatagaaaaaaaaagtattgacaggaaagaaaaagtagctgtatgcttataagtactttgaggatttgaattctctcttataaaacacaaacacaatttttagattttatttaaataatcatcaatccgattataattatttatatatttttctattttcaaagaagtaaatcatgagcttttccaactcaacatctattttttttctctcaacctttttcacatcttaagtagtctcaccctttatatatataacttatttcttaccttttacattatgtaacttttatcaccaaaaccaacaactttaaaattttattaaatagactccacaagtaacttgacactcttacattcatcgacattaacttttatctgttttataaatattattgtgatataatttaatcaaaataaccacaaactttcataaaaggttcttattaagcatggcatttaataagcaaaaacaactcaatcactttcatataggaggtagcctaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttcacaactcaaacattctctccattggtccttaaacactcatcagtcatcaccacatacagctagaaagctgtattgcctttagcactcaagctcaaaagacaactcagagttcaccatggccaagctagttttttccctttgttttctgcttttcagtggctgctgcttcgctggatcaattggcgccgcatctatggagttctgcttcgatgtttttaaagagcttaaagtgcaccatgccaacgagaatatcttctattgcccaattgccattatgtctgcccttgctatggtgtacttgggtgctaaagactctactaggacccagataaacaaggtagtcagattcgacaagctgcctgggtttggcgactctattgaagctcagtgtggtacttctgttaatgtccactcatccctccgcgacatacttaatcaaattacaaaaccaaatgatgtgtactcatttagtctggccagccgtttgtacgcagaggaacgctaccctatcctgccagagtatttgcaatgtgtgaaggaactttacaggggtgggcttgagccaataaactttcaaacagcagccgaccaagctagggagcttatcaattcttgggtcgagagccaaactaacggaatcatccgcaacgtcctccagccaagttccgttgattcccagaccgctatggtacttgtgaatgccattgtcttcaaggggctttgggagaaggcatttaaagacgaggacactcaggcaatgccctttcgtgtgaccgagcaggagtcaaaacctgttcaaatgatgtaccaaattgggctgttcagagttgctagtatggcctctgagaaaatgaagatccttgaactcccatttgcctccgggacaatgtctatgcttgtcctcctgccagatgaagtcagtgggctcgaacagctcgaaagcataataaactttgagaaacttaccgaatggacttcttccaatgttatggaggagcgtaaaattaaggtctatctgccccgcatgaaaatggaggaaaagtataatctcactagcgtcctcatggctatgggaattactgatgtattctcctctagcgctaatctgagtggaatctccagcgccgagtctctcaagataagccaggccgtgcacgctgctcatgctgaaatcaacgaagccggcagagaggtggtggggtcagctgaggcaggtgtagatgcagccagtgtctctgaggaatttagagccgatcaccctttccttttttgcattaaacatatcgctacaaatgccgttttgtttttcggtcgttgcgttagtccaaaggatgagctttaaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgactcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagt

SEQ ID NO: 765 - DNA, 4324 bp, synthetic construct
  misc_feature 1..4324: note = Construct AR15-24
  source 1..4324: mol_type = other DNA
aacacaagcttcaagttttaaaaggaaaaatgtcagccaaaaactttaaataaaatggtaacaaggaaattattcaaaaattacaaacctcgtcaaaataggaaagaaaaaaagtttagggatttagaaaaaacatcaatctagttccaccttattttatagagagaagaaactaatatataagaactaaaaaacagaagaatagaaaaaaaaagtattgacaggaaagaaaaagtagctgtatgcttataagtactttgaggatttgaattctctcttataaaacacaaacacaatttttagattttatttaaataatcatcaatccgattataattatttatatatttttctattttcaaagaagtaaatcatgagcttttccaactcaacatctattttttttctctcaacctttttcacatcttaagtagtctcaccctttatatatataacttatttcttaccttttacattatgtaacttttatcaccaaaaccaacaactttaaaattttattaaatagactccacaagtaacttgacactcttacattcatcgacattaacttttatctgttttataaatattattgtgatataatttaatcaaaataaccacaaactttcataaaaggttcttattaagcatggcatttaataagcaaaaacaactcaatcactttcatataggaggtagcctaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttcacaactcaaacattctctccattggtccttaaacactcatcagtcatcaccgaattctctaaaagagatctttttctgctctttgaagaaagaagggtctttgcttgattttggagatggccaagctagttttttccctttgttttctgcttttcagtggctgctgcttcgctggatcaattggcgccgcatctatggagttctgcttcgatgtttttaaagagcttaaagtgcaccatgccaacgagaatatcttctattgcccaattgccattatgtctgcccttgctatggtgtacttgggtgctaaagactctactaggacccagataaacaaggtagtcagattcgacaagctgcctgggtttggcgactctattgaagctcagtgtggtacttctgttaatgtccactcatccctccgcgacatacttaatcaaattacaaaaccaaatgatgtgtactcatttagtctggccagccgtttgtacgcagaggaacgctaccctatcctgccagagtatttgcaatgtgtgaaggaactttacaggggtgggcttgagccaataaactttcaaacagcagccgaccaagctagggagcttatcaattcttgggtcgagagccaaactaacggaatcatccgcaacgtcctccagccaagttccgttgattcccagaccgctatggtacttgtgaatgccattgtcttcaaggggctttgggagaaggcatttaaagacgaggacactcaggcaatgccctttcgtgtgaccgagcaggagtcaaaacctgttcaaatgatgtaccaaattgggctgttcagagttgctagtatggcctctgagaaaatgaagatccttgaactcccatttgcctccgggacaatgtctatgcttgtcctcctgccagatgaagtcagtgggctcgaacagctcgaaagcataataaactttgagaaacttaccgaatggacttcttccaatgttatggaggagcgtaaaattaaggtctatctgccccgcatgaaaatggaggaaaagtataatctcactagcgtcctcatggctatgggaattactgatgtattctcctctagcgctaatctgagtggaatctccagcgccgagtctctcaagataagccaggccgtgcacgctgctcatgctgaaatcaacgaagccggcagagaggtggtggggtcagctgaggcaggtgtagatgcagccagtgtctctgaggaatttagagccgatcaccctttccttttttgcattaaacatatcgctacaaatgccgttttgtttttcggtcgttgcgttagtccaaaggatgagctttaaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgactcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagt

SEQ ID NO: 766 - DNA, 4493 bp, synthetic construct
  misc_feature 1..4493: note = Construct AR15-38
  source 1..4493: mol_type = other DNA
aacacaagcttcaagttttaaaaggaaaaatgtcagccaaaaactttaaataaaatggtaacaaggaaattattcaaaaattacaaacctcgtcaaaataggaaagaaaaaaagtttagggatttagaaaaaacatcaatctagttccaccttattttatagagagaagaaactaatatataagaactaaaaaacagaagaatagaaaaaaaaagtattgacaggaaagaaaaagtagctgtatgcttataagtactttgaggatttgaattctctcttataaaacacaaacacaatttttagattttatttaaataatcatcaatccgattataattatttatatatttttctattttcaaagaagtaaatcatgagcttttccaactcaacatctattttttttctctcaacctttttcacatcttaagtagtctcaccctttatatatataacttatttcttaccttttacattatgtaacttttatcaccaaaaccaacaactttaaaattttattaaatagactccacaagtaacttgacactcttacattcatcgacattaacttttatctgttttataaatattattgtgatataatttaatcaaaataaccacaaactttcataaaaggttcttattaagcatggcatttaataagcaaaaacaactcaatcactttcatataggaggtagcctaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttcacaactcaaacattctctccattggtccttaaacactcatcagtcatcaccatgcagattttcgtgaagaccttaacggggaagacgatcaccctagaggttgagtcttccgacaccatcgacaatgtcaaagccaagatccaggacaaggaagggatacccccagaccagcagcgtttgattttcgccggaaagcagcttgaggatggtcgtactcttgccgactacaacatccagaaggagtcaactctccatctcgtgctccgtctccgtggtggtggttccatggccaagctagttttttccctttgttttctgcttttcagtggctgctgcttcgctggatcaattggcgccgcatctatggagttctgcttcgatgtttttaaagagcttaaagtgcaccatgccaacgagaatatcttctattgcccaattgccattatgtctgcccttgctatggtgtacttgggtgctaaagactctactaggacccagataaacaaggtagtcagattcgacaagctgcctgggtttggcgactctattgaagctcagtgtggtacttctgttaatgtccactcatccctccgcgacatacttaatcaaattacaaaaccaaatgatgtgtactcatttagtctggccagccgtttgtacgcagaggaacgctaccctatcctgccagagtatttgcaatgtgtgaaggaactttacaggggtgggcttgagccaataaactttcaaacagcagccgaccaagctagggagcttatcaattcttgggtcgagagccaaactaacggaatcatccgcaacgtcctccagccaagttccgttgattcccagaccgctatggtacttgtgaatgccattgtcttcaaggggctttgggagaaggcatttaaagacgaggacactcaggcaatgccctttcgtgtgaccgagcaggagtcaaaacctgttcaaatgatgtaccaaattgggctgttcagagttgctagtatggcctctgagaaaatgaagatccttgaactcccatttgcctccgggacaatgtctatgcttgtcctcctgccagatgaagtcagtgggctcgaacagctcgaaagcataataaactttgagaaacttaccgaatggacttcttccaatgttatggaggagcgtaaaattaaggtctatctgccccgcatgaaaatggaggaaaagtataatctcactagcgtcctcatggctatgggaattactgatgtattctcctctagcgctaatctgagtggaatctccagcgccgagtctctcaagataagccaggccgtgcacgctgctcatgctgaaatcaacgaagccggcagagaggtggtggggtcagctgaggcaggtgtagatgcagccagtgtctctgaggaatttagagccgatcaccctttccttttttgcattaaacatatcgctacaaatgccgttttgtttttcggtcgttgcgttagtccaaaggatgagctttaaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgactcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagt

SEQ ID NO: 767 - DNA, 1956 bp, synthetic construct
  misc_feature 1..1956: note = Construct AR07-28
  source 1..1956: mol_type = other DNA
catcggtgattgattcctttaaagacttatgtttcttatcttgcttctgaggcaagtattcagttaccagttaccacttatattctggactttctgactgcatcctcatttttccaacattttaaatttcactattggctgaatgcttcttctttgaggaagaaacaattcagatggcagaaatgtatcaaccaatgcatatatacaaatgtacctcttgttctcaaaacatctatcggatggttccatttgctttgtcatccaattagtgactactttatattattcactcctctttattactattttcatgcgaggttgccatgtacattatatttgtaaggattgacgctattgagcgtttttcttcaattttctttattttagacatgggtatgaaatgtgtgttagagttgggttgaatgagatatacgttcaagtgaagtggcataccgttgtcgagtaaggatgacctacccattcttgagacaaatgttacattttagtatcagagtaaaatgtgtacctataactcaaattcgattgacatgtatccattcaacataaaattaaaccagcctgcacctgcatccacatttcaagtattttcaaaccgttcggctcctatccaccgggtgtaacaagacggattccgaatttggaagattttgactcaaattcccaatttatattgaccgtgactaaatcaactttaacttctataattctgattaagctcccaatttatattcccaacggcactacctccaaaatttatagactctcatccccttttaaaccaacttagtaaacgtttttttttttaattttatgaagttaagtttttaccttgtttttaaaaagaatcgttcataagatgccatgccagaacattagctacacgttacacatagcatgcagccgcggagaattgtttttcttcgccacttgtcactcccttcaaacacctaagagcttctctctcacagcacacacatacaatcacatgcgtgcatgcattattacacgtgatcgccatgcaaatctcctttatagcctataaattaactcatccgcttcactctttactcaaaccaaaactcatcaatacaaacaagattaaaaacataatgatgagagcacggttcccattactgttgctgggacttgttttcctggcttcagtttctgtctcattgatcgtaacacagactatgaagggtcttgatatacagaaggtggccgggacttggtacagtttggcaatggccgcatccgacatctccttgttggacgcacaatcagccccattgcgtgtgtacgtagaagagcttaaaccaactcccgagggggatctggaaattctgctccagaaatgggagaacggtgagtgcgcccagaagaagatcatcgcagagaagaccaaaattccagcagtattcaaaatcgacgcattgaacgaaaataaggtgctcgtactggacactgattataagaagtatctccttttctgtatggagaactcagcagagcctgaacagagtcttgcctgccaatgccttgttcgtaccccagaggtagatgatgaagctctggaaaagttcgataaggcccttaaggctctgcctatgcacattaggctttctttcaatccaactcaacttgaggaacaatgtcacattaaggatgagctttaaagatctgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 768 - DNA, 2201 bp, synthetic construct
  misc_feature 1..2201: note = Construct AR07-29
  source 1..2201: mol_type = other DNA
aacacaagcttcaagttttaaaaggaaaaatgtcagccaaaaactttaaataaaatggtaacaaggaaattattcaaaaattacaaacctcgtcaaaataggaaagaaaaaaagtttagggatttagaaaaaacatcaatctagttccaccttattttatagagagaagaaactaatatataagaactaaaaaacagaagaatagaaaaaaaaagtattgacaggaaagaaaaagtagctgtatgcttataagtactttgaggatttgaattctctcttataaaacacaaacacaatttttagattttatttaaataatcatcaatccgattataattatttatatatttttctattttcaaagaagtaaatcatgagcttttccaactcaacatctattttttttctctcaacctttttcacatcttaagtagtctcaccctttatatatataacttatttcttaccttttacattatgtaacttttatcaccaaaaccaacaactttaaaattttattaaatagactccacaagtaacttgacactcttacattcatcgacattaacttttatctgttttataaatattattgtgatataatttaatcaaaataaccacaaactttcataaaaggttcttattaagcatggcatttaataagcaaaaacaactcaatcactttcatataggaggtagcctaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttcacaactcaaacattctctccattggtccttaaacactcatcagtcatcaccatggccaagctagttttttccctttgttttctgcttttcagtggctgctgcttcgctttgatcgtaacacagactatgaagggtcttgatatacagaaggtggccgggacttggtacagtttggcaatggccgcatccgacatctccttgttggacgcacaatcagccccattgcgtgtgtacgtagaagagcttaaaccaactcccgagggggatctggaaattctgctccagaaatgggagaacggtgagtgcgcccagaagaagatcatcgcagagaagaccaaaattccagcagtattcaaaatcgacgcattgaacgaaaataaggtgctcgtactggacactgattataagaagtatctccttttctgtatggagaactcagcagagcctgaacagagtcttgcctgccaatgccttgttcgtaccccagaggtagatgatgaagctctggaaaagttcgataaggcccttaaggctctgcctatgcacattaggctttctttcaatccaactcaacttgaggaacaatgtcacattaaggatgagctttaaagatctgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 769 - DNA, 2277 bp, synthetic construct
  misc_feature 1..2277: note = Construct AR07-31
  source 1..2277: mol_type = other DNA
caactattatcgcatgatgatgtacgttaagtcatcatcatctttaactttatatattgttaaaagtagaaaaaataggtgatgcattataaaataattttataacatcatttaattataaattatttataataaatatttgagtttttatagtaattacctaaacaattatatcaagactaatgcctgattagttgacatgacgaaattaaactcataaaagtaaagatgtttatgtggaaaactcttatacaattgagcggacttttttccatggtagttcagttttcttctattcaatttatttttttggtttccgctcagaataagaataatttgataaattcatttttaggcaattaagaatatttatttgactaactttttaattgaaataaatttacaataaatactcaatttatctttcacaatcaaaagattgagatgttgtaagatctccgataatatacttatatcttttcatttattacgttttcaaatttgaattttaatgtgtgttgtaagtataaatttaaaataaaaataaaaacaattattatatcaaaatggcaaaaacatttaatacgtattatttaagaaaaaaatatgtaataatatatttatattttaatatctattcttatgtattttttaaaaatctattatatattgatcaactaaaatatttttatatctacacttattttgcatttttatcaattttcttgcgttttttggcatatttaataatgactattctttaataatcaatcattattcttacatggtacatattgttggaaccatatgaagtgtccattgcatttgactatgtggatagtgttttgatccaggcctccatttgccgcttattaattaatttggtaacagtccgtactaatcagttacttatccttcctccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagcccacacatttgttgtttctaacccaacctcaaactcgtattctcttccgccacctcatttttgtttatttcaacacccgtcaaactgcatgccaccccgtggccaaatgtccatgcatgttaacaagacctatgactataaatatctgcaatctcggcccaggttttcatcatcaagaaccagttcaatatcctagtacaccgtattaaagaatttaagatatactatggctaccaagatatttgccctccttgtgctccttgctctttcagcgagcgctacaactgcgttgatcgtaacacagactatgaagggtcttgatatacagaaggtggccgggacttggtacagtttggcaatggccgcatccgacatctccttgttggacgcacaatcagccccattgcgtgtgtacgtagaagagcttaaaccaactcccgagggggatctggaaattctgctccagaaatgggagaacggtgagtgcgcccagaagaagatcatcgcagagaagaccaaaattccagcagtattcaaaatcgacgcattgaacgaaaataaggtgctcgtactggacactgattataagaagtatctccttttctgtatggagaactcagcagagcctgaacagagtcttgcctgccaatgccttgttcgtaccccagaggtagatgatgaagctctggaaaagttcgataaggcccttaaggctctgcctatgcacattaggctttctttcaatccaactcaacttgaggaacaatgtcacattaaggatgagctttaaagatctgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 770 - DNA, 2286 bp, synthetic construct
  misc_feature 1..2286: note = Construct AR07-32
  source 1..2286: mol_type = other DNA
caactattatcgcatgatgatgtacgttaagtcatcatcatctttaactttatatattgttaaaagtagaaaaaataggtgatgcattataaaataattttataacatcatttaattataaattatttataataaatatttgagtttttatagtaattacctaaacaattatatcaagactaatgcctgattagttgacatgacgaaattaaactcataaaagtaaagatgtttatgtggaaaactcttatacaattgagcggacttttttccatggtagttcagttttcttctattcaatttatttttttggtttccgctcagaataagaataatttgataaattcatttttaggcaattaagaatatttatttgactaactttttaattgaaataaatttacaataaatactcaatttatctttcacaatcaaaagattgagatgttgtaagatctccgataatatacttatatcttttcatttattacgttttcaaatttgaattttaatgtgtgttgtaagtataaatttaaaataaaaataaaaacaattattatatcaaaatggcaaaaacatttaatacgtattatttaagaaaaaaatatgtaataatatatttatattttaatatctattcttatgtattttttaaaaatctattatatattgatcaactaaaatatttttatatctacacttattttgcatttttatcaattttcttgcgttttttggcatatttaataatgactattctttaataatcaatcattattcttacatggtacatattgttggaaccatatgaagtgtccattgcatttgactatgtggatagtgttttgatccaggcctccatttgccgcttattaattaatttggtaacagtccgtactaatcagttacttatccttcctccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagcccacacatttgttgtttctaacccaacctcaaactcgtattctcttccgccacctcatttttgtttatttcaacacccgtcaaactgcatgccaccccgtggccaaatgtccatgcatgttaacaagacctatgactataaatatctgcaatctcggcccaggttttcatcatcaagaaccagttcaatatcctagtacaccgtattaaagaatttaagatatactatgatgagagcgcggttcccattactgttgctgggagttgttttcctggcatcagtttctgtctcatttggcttgatcgtaacacagactatgaagggtcttgatatacagaaggtggccgggacttggtacagtttggcaatggccgcatccgacatctccttgttggacgcacaatcagccccattgcgtgtgtacgtagaagagcttaaaccaactcccgagggggatctggaaattctgctccagaaatgggagaacggtgagtgcgcccagaagaagatcatcgcagagaagaccaaaattccagcagtattcaaaatcgacgcattgaacgaaaataaggtgctcgtactggacactgattataagaagtatctccttttctgtatggagaactcagcagagcctgaacagagtcttgcctgccaatgccttgttcgtaccccagaggtagatgatgaagctctggaaaagttcgataaggcccttaaggctctgcctatgcacattaggctttctttcaatccaactcaacttgaggaacaatgtcacattaaggatgagctttaaagatctgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 771 - DNA, 3350 bp, synthetic construct
  misc_feature 1..3350: note = Construct AR07-33
  source 1..3350: mol_type = other DNA
cattgtactcccagtatcattatagtgaaagttttggctctctcgccggtggttttttacctctatttaaaggggttttccacctaaaaattctggtatcattctcactttacttgttactttaatttctcataatctttggttgaaattatcacgcttccgcacacgatatccctacaaatttattatttgttaaacattttcaaaccgcataaaattttatgaagtcccgtctatctttaatgtagtctaacattttcatattgaaatatataatttacttaattttagcgttggtagaaagcataatgatttattcttattcttcttcatataaatgtttaatatacaatataaacaaattctttaccttaagaaggatttcccattttatattttaaaaatatatttatcaaatatttttcaaccacgtaaatcacataataataagttgtttcaaaagtaataaaatttaactccataatttttttatttgactgatcttaaagcaacacccagtgacacaactagccatttttttctttgaataaaaaaatccaattatcattgtattttttttatacaatgaaaatttcaccaaacaatgatttgtggtatttctgaagcaagtcatgttatgcaaaattctataattcccatttgacactacggaagtaactgaagatctgcttttacatgcgagacacatcttctaaagtaattttaataatagttactatattcaagatttcatatatcaaatactcaatattacttctaaaaaattaattagatataattaaaatattacttttttaattttaagtttaattgttgaatttgtgactattgatttattattctactatgtttaaattgttttataggtagtttaaagtaaatataagtaatgtagtagagtgttagagtgttaccctaaaccataaactataagatttatggtggactaattttcatatatttcttattgcttttaccttttcttggtatgtaagtccgtaactggaattactgtgggttgccatgacactctgtggtcttttggttcatgcatggatgcttgcgcaagaaaaagacaaagaacaaagaaaaaagacaaaacagagagacaaaacgcaatcacacaaccaactcaaattagtcactggctgatcaagatcgccgcgtccatgtatgtctaaatgccatgcaaagcaacacgtgcttaacatgcactttaaatggctcacccatcccaacccactcacaaacacattgcctttttcttcatcatcaccacaaccacctgtatatattcattctcttccgccacctcaatttcttcacttcaacacacgtcaacctgcatatgcgtgtcatcccatgcccaaatctccatgcatgttcctaccaccttctctcttatataatacctataaatacctctaatatcactcacttctttcatcatccatccatccagagtactactactctactactataataccccaacccaactcatattcaatactactctacttgaatgcatgatcatggctacttcaaagttgaaaacccagaatgtggttgtatctctctccctaaccttaaccttggtactggtgctactgaccagcaaggcaaactcattgatcgtaacacagactatgaagggtcttgatatacagaaggtggccgggacttggtacagtttggcaatggccgcatccgacatctccttgttggacgcacaatcagccccattgcgtgtgtacgtagaagagcttaaaccaactcccgagggggatctggaaattctgctccagaaatgggagaacggtgagtgcgcccagaagaagatcatcgcagagaagaccaaaattccagcagtattcaaaatcgacgcattgaacgaaaataaggtgctcgtactggacactgattataagaagtatctccttttctgtatggagaactcagcagagcctgaacagagtcttgcctgccaatgccttgttcgtaccccagaggtagatgatgaagctctggaaaagttcgataaggcccttaaggctctgcctatgcacattaggctttctttcaatccaactcaacttgaggaacaatgtcacattaaggatgagctttaaaataaataaaatgggagcaataaataaaatgggagctcatatatttacaccatttacactgtctattattcaccatgccaattattacttcataattttaaaattatgtcatttttaaaaattgcttaatgatggaaaggattattataagttaaaagtataacatagataaactaaccacaaaacaaatcaatataaactaacttactctcccatctaatttttatttaaatttctttacacttctcttccatttctatttctacaacattatttaacatttttattgtatttttcttactttctaactctattcatttcaaaaatcaatatatgtttatcaccacctctctaaaaaaaactttacaatcattggtccagaaaagttaaatcacgagatggtcattttagcattaaaacaacgattcttgtatcactatttttcagcatgtagtccattctcttcaaacaaagacagcggctatataatcgttgtgttatattcagtctaaaacaattgttatggtaaaagtcgtcattttacgcctttttaaaagatataaaatgacagttatggttaaaagtcatcatgttagatcctccttaaagatataaaatgacagttttggataaaaagtggtcattttatacgctcttgaaagatataaaacgacggttatggtaaaagctgccattttaaatgaaatatttttgttttagttcattttgtttaatgctaatcccatttaaattgacttgtacaattaaaactcacccacccagatacaatataaactaacttactctcacagctaagttttatttaaatttctttacacttcttttccatttctatttctatgacattaactaacatttttctcgtaattttttttcttattttctaactctatccatttcaaatcgatatatgtttatcaccaccactttaaaaagaaaatttacaatttctcgtgcaaaaaagctaaatcatgaccgtcattttagcattaaaacaacgattcttgtatcgttgtttttcagcatgtagtccattcttttcaagcaaagacaacagctatataatcatcgtgttatattcagtctaaaacaacagtaatgataaaagtcatcattttaggcctttctgaaatatatagaacgacattcatggtaaaaaatcgtcattttagatcc

SEQ ID NO: 772 - DNA, 3590 bp, synthetic construct
  misc_feature 1..3590: note = Construct AR15-25
  source 1..3590: mol_type = other DNA
aacacaagcttcaagttttaaaaggaaaaatgtcagccaaaaactttaaataaaatggtaacaaggaaattattcaaaaattacaaacctcgtcaaaataggaaagaaaaaaagtttagggatttagaaaaaacatcaatctagttccaccttattttatagagagaagaaactaatatataagaactaaaaaacagaagaatagaaaaaaaaagtattgacaggaaagaaaaagtagctgtatgcttataagtactttgaggatttgaattctctcttataaaacacaaacacaatttttagattttatttaaataatcatcaatccgattataattatttatatatttttctattttcaaagaagtaaatcatgagcttttccaactcaacatctattttttttctctcaacctttttcacatcttaagtagtctcaccctttatatatataacttatttcttaccttttacattatgtaacttttatcaccaaaaccaacaactttaaaattttattaaatagactccacaagtaacttgacactcttacattcatcgacattaacttttatctgttttataaatattattgtgatataatttaatcaaaataaccacaaactttcataaaaggttcttattaagcatggcatttaataagcaaaaacaactcaatcactttcatataggaggtagcctaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttcacaactcaaacattctctccattggtccttaaacactcatcagtcatcaccatggccaagctagttttttccctttgttttctgcttttcagtggctgctgcttcgctcttattgtgacccaaaccatgaagggcctcgacattcaaaaggttgccggaacctggtactcccttgctatggctgcttccgatatctccttgctcgatgctcaatccgctccacttagggtgtacgtggaagagttgaagccaactccagagggcgatcttgagatcttgcttcaaaagtgggagaacgatgagtgcgcccagaagaagattatcgccgaaaagaccaagattcccgccgtgttcaagatcgatgctctcaacgagaacaaggtgctcgtgctcgataccgactacaagaagtaccttctcgtctgcatggaaaactccgctgagccagagcaatctcttgtttgccaatgccttgtgaggaccccagaggttgacgatgaagctcttgagaagttcgacaaggctctcaaggctttgcctatgcacatccgccttagcttcaacccaactcagcttgaggaacagtgccacatcaaggatgagctttaaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgactcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagt

SEQ ID NO: 773 - DNA, 3590 bp, synthetic construct
  misc_feature 1..3590: note = Construct AR15-26
  source 1..3590: mol_type = other DNA
aacacaagcttcaagttttaaaaggaaaaatgtcagccaaaaactttaaataaaatggtaacaaggaaattattcaaaaattacaaacctcgtcaaaataggaaagaaaaaaagtttagggatttagaaaaaacatcaatctagttccaccttattttatagagagaagaaactaatatataagaactaaaaaacagaagaatagaaaaaaaaagtattgacaggaaagaaaaagtagctgtatgcttataagtactttgaggatttgaattctctcttataaaacacaaacacaatttttagattttatttaaataatcatcaatccgattataattatttatatatttttctattttcaaagaagtaaatcatgagcttttccaactcaacatctattttttttctctcaacctttttcacatcttaagtagtctcaccctttatatatataacttatttcttaccttttacattatgtaacttttatcaccaaaaccaacaactttaaaattttattaaatagactccacaagtaacttgacactcttacattcatcgacattaacttttatctgttttataaatattattgtgatataatttaatcaaaataaccacaaactttcataaaaggttcttattaagcatggcatttaataagcaaaaacaactcaatcactttcatataggaggtagcctaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttcacaactcaaacattctctccattggtccttaaacactcatcagtcatcaccatggccaagctagttttttccctttgttttctgcttttcagtggctgctgcttcgctctcattgttacacaaaccatgaagggtcttgacattcagaaggttgctgggacatggtattcactagcgatggctgcttctgatatctccctgttggatgcacagtctgcccccctgagagtgtatgttgaagaactgaaaccgacacctgaaggagacttggaaattttactccagaaatgggaaaatgatgagtgtgcccaaaagaagataatagccgagaagaccaaaattcctgctgtgtttaagattgatgctttgaatgagaacaaagtactagtcctcgacactgattacaagaaatacttattagtgtgcatggaaaacagcgcagagccagaacaatcacttgtttgtcaatgtttggtccgtactccagaggtagatgatgaagcattggagaaatttgataaagcattgaaggcacttccaatgcatataaggcttagtttcaatcctactcagcttgaagagcaatgccacatcaaggatgagctttaaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgactcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagt

SEQ ID NO: 774 - DNA, 3590 bp, synthetic construct
  misc_feature 1..3590: note = Construct AR15-27
  source 1..3590: mol_type = other DNA
aacacaagcttcaagttttaaaaggaaaaatgtcagccaaaaactttaaataaaatggtaacaaggaaattattcaaaaattacaaacctcgtcaaaataggaaagaaaaaaagtttagggatttagaaaaaacatcaatctagttccaccttattttatagagagaagaaactaatatataagaactaaaaaacagaagaatagaaaaaaaaagtattgacaggaaagaaaaagtagctgtatgcttataagtactttgaggatttgaattctctcttataaaacacaaacacaatttttagattttatttaaataatcatcaatccgattataattatttatatatttttctattttcaaagaagtaaatcatgagcttttccaactcaacatctattttttttctctcaacctttttcacatcttaagtagtctcaccctttatatatataacttatttcttaccttttacattatgtaacttttatcaccaaaaccaacaactttaaaattttattaaatagactccacaagtaacttgacactcttacattcatcgacattaacttttatctgttttataaatattattgtgatataatttaatcaaaataaccacaaactttcataaaaggttcttattaagcatggcatttaataagcaaaaacaactcaatcactttcatataggaggtagcctaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttcacaactcaaacattctctccattggtccttaaacactcatcagtcatcaccatggccaagctagttttttccctttgttttctgcttttcagtggctgctgcttcgctcttatagtaactcaaaccatgaagggacttgatatccaaaaagttgcaggaacctggtactcactggctatggcagcttccgacatctccttgttggacgcacaatccgcaccattgcgcgtctacgttgaggagttgaaacctacaccagagggggatcttgagattttgctccagaaatgggagaacgacgagtgtgcccagaaaaaaattatagcagagaagactaaaattcctgctgtttttaagattgatgccctgaacgagaataaggtactggtcctcgacactgattataaaaagtatttgctggtgtgtatggagaacagtgctgaacctgaacagagcctggtctgtcaatgtcttgtaaggacacctgaggttgatgacgaggcacttgaaaaattcgacaaggcccttaaggctctgcctatgcacatccgtctgagtttcaaccctactcagttggaggaacaatgtcatattaaggatgagctttaaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgactcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagt

SEQ ID NO: 775 - DNA, 3578 bp, synthetic construct
  misc_feature 1..3578: note = Construct AR15-28
  source 1..3578: mol_type = other DNA
aacacaagcttcaagttttaaaaggaaaaatgtcagccaaaaactttaaataaaatggtaacaaggaaattattcaaaaattacaaacctcgtcaaaataggaaagaaaaaaagtttagggatttagaaaaaacatcaatctagttccaccttattttatagagagaagaaactaatatataagaactaaaaaacagaagaatagaaaaaaaaagtattgacaggaaagaaaaagtagctgtatgcttataagtactttgaggatttgaattctctcttataaaacacaaacacaatttttagattttatttaaataatcatcaatccgattataattatttatatatttttctattttcaaagaagtaaatcatgagcttttccaactcaacatctattttttttctctcaacctttttcacatcttaagtagtctcaccctttatatatataacttatttcttaccttttacattatgtaacttttatcaccaaaaccaacaactttaaaattttattaaatagactccacaagtaacttgacactcttacattcatcgacattaacttttatctgttttataaatattattgtgatataatttaatcaaaataaccacaaactttcataaaaggttcttattaagcatggcatttaataagcaaaaacaactcaatcactttcatataggaggtagcctaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttcacaactcaaacattctctccattggtccttaaacactcatcagtcatcaccatggccaagctagttttttccctttgttttctgcttttcagtggctgctgcttcgctcttattgtgacccaaaccatgaagggcctcgacattcaaaaggttgccggaacctggtactcccttgctatggctgcttccgatatctccttgctcgatgctcaatccgctccacttagggtgtacgtggaagagttgaagccaactccagagggcgatcttgagatcttgcttcaaaagtgggagaacgatgagtgcgcccagaagaagattatcgccgaaaagaccaagattcccgccgtgttcaagatcgatgctctcaacgagaacaaggtgctcgtgctcgataccgactacaagaagtaccttctcgtctgcatggaaaactccgctgagccagagcaatctcttgtttgccaatgccttgtgaggaccccagaggttgacgatgaagctcttgagaagttcgacaaggctctcaaggctttgcctatgcacatccgccttagcttcaacccaactcagcttgaggaacagtgccacatctaaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgactcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagt

SEQ ID NO: 776 - DNA, 4371 bp, synthetic construct
  misc_feature 1..4371: note = Construct AR15-29
  source 1..4371: mol_type = other DNA
aacacaagcttcaagttttaaaaggaaaaatgtcagccaaaaactttaaataaaatggtaacaaggaaattattcaaaaattacaaacctcgtcaaaataggaaagaaaaaaagtttagggatttagaaaaaacatcaatctagttccaccttattttatagagagaagaaactaatatataagaactaaaaaacagaagaatagaaaaaaaaagtattgacaggaaagaaaaagtagctgtatgcttataagtactttgaggatttgaattctctcttataaaacacaaacacaatttttagattttatttaaataatcatcaatccgattataattatttatatatttttctattttcaaagaagtaaatcatgagcttttccaactcaacatctattttttttctctcaacctttttcacatcttaagtagtctcaccctttatatatataacttatttcttaccttttacattatgtaacttttatcaccaaaaccaacaactttaaaattttattaaatagactccacaagtaacttgacactcttacattcatcgacattaacttttatctgttttataaatattattgtgatataatttaatcaaaataaccacaaactttcataaaaggttcttattaagcatggcatttaataagcaaaaacaactcaatcactttcatataggaggtagcctaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttcacaactcaaacattctctccattggtccttaaacactcatcagtcatcaccgttcgttatctaccaccgttctatggattttattccttctattcgtgtttattctattggtttatgttgcttgcaatatgttttttctgaatctgtcgtcgttgtcttcaattttatccatgtttcagagatcaattttgtttgtgtagtatgtgcttattcttcttcttttcgttcgagttgttaataacggtgctatggtgttttcaaaagtgttttttttattacttttgatttaaagtttttttggtaaggcttttatttgcttgttatattcaaatctttggatccagatcttatataagtttttggttcaagaaagtttttggttactgatgaatagatctattaactgttactttaatcgattcaagctaaagttttttggttactgatgaatagatctattatctgttacttttaatcggttcaagctcaagttttttggttactgatgaatagatctatatacgtcacagtgtgctaaacatgcccttgttttatctcgatcttatgtatgggagtgccataaattttgttatgtctatttttttatctgttggaatcatactgagtttgatgcgttacgattgagcatacctatttttgggcttgttgtatggtgggtatttagatcttaatctttttatgcttatgaaaggttttgtaatgacaaaggtcttaatgttgttaaacttttatttttactttatatggtgtgttgatgtgttatggttttgacaactttttttttttctggatttttgcagatttaaggaccatggccaagctagttttttccctttgttttctgcttttcagtggctgctgcttcgctcttattgtgacccaaaccatgaagggcctcgacattcaaaaggttgccggaacctggtactcccttgctatggctgcttccgatatctccttgctcgatgctcaatccgctccacttagggtgtacgtggaagagttgaagccaactccagagggcgatcttgagatcttgcttcaaaagtgggagaacgatgagtgcgcccagaagaagattatcgccgaaaagaccaagattcccgccgtgttcaagatcgatgctctcaacgagaacaaggtgctcgtgctcgataccgactacaagaagtaccttctcgtctgcatggaaaactccgctgagccagagcaatctcttgtttgccaatgccttgtgaggaccccagaggttgacgatgaagctcttgagaagttcgacaaggctctcaaggctttgcctatgcacatccgccttagcttcaacccaactcagcttgaggaacagtgccacatcaaggatgagctttaaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgactcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagt

SEQ ID NO: 777 - DNA, 4360 bp, synthetic construct
  misc_feature 1..4360: note = Construct AR15-30
  source 1..4360: mol_type = other DNA
aacacaagcttcaagttttaaaaggaaaaatgtcagccaaaaactttaaataaaatggtaacaaggaaattattcaaaaattacaaacctcgtcaaaataggaaagaaaaaaagtttagggatttagaaaaaacatcaatctagttccaccttattttatagagagaagaaactaatatataagaactaaaaaacagaagaatagaaaaaaaaagtattgacaggaaagaaaaagtagctgtatgcttataagtactttgaggatttgaattctctcttataaaacacaaacacaatttttagattttatttaaataatcatcaatccgattataattatttatatatttttctattttcaaagaagtaaatcatgagcttttccaactcaacatctattttttttctctcaacctttttcacatcttaagtagtctcaccctttatatatataacttatttcttaccttttacattatgtaacttttatcaccaaaaccaacaactttaaaattttattaaatagactccacaagtaacttgacactcttacattcatcgacattaacttttatctgttttataaatattattgtgatataatttaatcaaaataaccacaaactttcataaaaggttcttattaagcatggcatttaataagcaaaaacaactcaatcactttcatataggaggtagcctaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttcacaactcaaacattctctccattggtccttaaacactcatcagtcatcaccatggccaagctagttttttccctttgttttctgcttttcagtggctgctgcttcgctcttattgtgacccaaaccatgaagggcctcgacattcaaaaggttcgttatctaccaccgttctatggattttattccttctattcgtgtttattctattggtttatgttgcttgcaatatgttttttctgaatctgtcgtcgttgtcttcaattttatccatgtttcagagatcaattttgtttgtgtagtatgtgcttattcttcttcttttcgttcgagttgttaataacggtgctatggtgttttcaaaagtgttttttttattacttttgatttaaagtttttttggtaaggcttttatttgcttgttatattcaaatctttggatccagatcttatataagtttttggttcaagaaagtttttggttactgatgaatagatctattaactgttactttaatcgattcaagctaaagttttttggttactgatgaatagatctattatctgttacttttaatcggttcaagctcaagttttttggttactgatgaatagatctatatacgtcacagtgtgctaaacatgcccttgttttatctcgatcttatgtatgggagtgccataaattttgttatgtctatttttttatctgttggaatcatactgagtttgatgcgttacgattgagcatacctatttttgggcttgttgtatggtgggtatttagatcttaatctttttatgcttatgaaaggttttgtaatgacaaaggtcttaatgttgttaaacttttatttttactttatatggtgtgttgatgtgttatggttttgacaactttttttttttctggatttttgcaggttgccggaacctggtactcccttgctatggctgcttccgatatctccttgctcgatgctcaatccgctccacttagggtgtacgtggaagagttgaagccaactccagagggcgatcttgagatcttgcttcaaaagtgggagaacgatgagtgcgcccagaagaagattatcgccgaaaagaccaagattcccgccgtgttcaagatcgatgctctcaacgagaacaaggtgctcgtgctcgataccgactacaagaagtaccttctcgtctgcatggaaaactccgctgagccagagcaatctcttgtttgccaatgccttgtgaggaccccagaggttgacgatgaagctcttgagaagttcgacaaggctctcaaggctttgcctatgcacatccgccttagcttcaacccaactcagcttgaggaacagtgccacatcaaggatgagctttaaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgactcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagt

SEQ ID NO: 778 - DNA, 3683 bp, synthetic construct
  misc_feature 1..3683: note = Construct AR15-31
  source 1..3683: mol_type = other DNA
aacacaagcttcaagttttaaaaggaaaaatgtcagccaaaaactttaaataaaatggtaacaaggaaattattcaaaaattacaaacctcgtcaaaataggaaagaaaaaaagtttagggatttagaaaaaacatcaatctagttccaccttattttatagagagaagaaactaatatataagaactaaaaaacagaagaatagaaaaaaaaagtattgacaggaaagaaaaagtagctgtatgcttataagtactttgaggatttgaattctctcttataaaacacaaacacaatttttagattttatttaaataatcatcaatccgattataattatttatatatttttctattttcaaagaagtaaatcatgagcttttccaactcaacatctattttttttctctcaacctttttcacatcttaagtagtctcaccctttatatatataacttatttcttaccttttacattatgtaacttttatcaccaaaaccaacaactttaaaattttattaaatagactccacaagtaacttgacactcttacattcatcgacattaacttttatctgttttataaatattattgtgatataatttaatcaaaataaccacaaactttcataaaaggttcttattaagcatggcatttaataagcaaaaacaactcaatcactttcatataggaggtagcctaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttcacaactcaaacattctctccattggtccttaaacactcatcagtcatcaccatggccaagctagttttttccctttgttttctgcttttcagtggctgctgcttcgctcttattgtgacccaaaccatgaagggcctcgacattcaaaaggtaaccatatctttcatctgttatgtgactacacattgcttctctttttgtgttctgtctcattaattgcggtttgttacatgttgtttgtaggttgccggaacctggtactcccttgctatggctgcttccgatatctccttgctcgatgctcaatccgctccacttagggtgtacgtggaagagttgaagccaactccagagggcgatcttgagatcttgcttcaaaagtgggagaacgatgagtgcgcccagaagaagattatcgccgaaaagaccaagattcccgccgtgttcaagatcgatgctctcaacgagaacaaggtgctcgtgctcgataccgactacaagaagtaccttctcgtctgcatggaaaactccgctgagccagagcaatctcttgtttgccaatgccttgtgaggaccccagaggttgacgatgaagctcttgagaagttcgacaaggctctcaaggctttgcctatgcacatccgccttagcttcaacccaactcagcttgaggaacagtgccacatcaaggatgagctttaaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgactcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagt

SEQ ID NO: 779 - DNA, 3655 bp, synthetic construct
  misc_feature 1..3655: note = Construct AR15-36
  source 1..3655: mol_type = other DNA
aacacaagcttcaagttttaaaaggaaaaatgtcagccaaaaactttaaataaaatggtaacaaggaaattattcaaaaattacaaacctcgtcaaaataggaaagaaaaaaagtttagggatttagaaaaaacatcaatctagttccaccttattttatagagagaagaaactaatatataagaactaaaaaacagaagaatagaaaaaaaaagtattgacaggaaagaaaaagtagctgtatgcttataagtactttgaggatttgaattctctcttataaaacacaaacacaatttttagattttatttaaataatcatcaatccgattataattatttatatatttttctattttcaaagaagtaaatcatgagcttttccaactcaacatctattttttttctctcaacctttttcacatcttaagtagtctcaccctttatatatataacttatttcttaccttttacattatgtaacttttatcaccaaaaccaacaactttaaaattttattaaatagactccacaagtaacttgacactcttacattcatcgacattaacttttatctgttttataaatattattgtgatataatttaatcaaaataaccacaaactttcataaaaggttcttattaagcatggcatttaataagcaaaaacaactcaatcactttcatataggaggtagcctaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttcacaactcaaacattctctccattggtccttaaacactcatcagtcatcacccccgagcccgctgtctcagccctccactccctgcagagctcagaagcgtgaccccagctgcagccatggccaagctagttttttccctttgttttctgcttttcagtggctgctgcttcgctcttattgtgacccaaaccatgaagggcctcgacattcaaaaggttgccggaacctggtactcccttgctatggctgcttccgatatctccttgctcgatgctcaatccgctccacttagggtgtacgtggaagagttgaagccaactccagagggcgatcttgagatcttgcttcaaaagtgggagaacgatgagtgcgcccagaagaagattatcgccgaaaagaccaagattcccgccgtgttcaagatcgatgctctcaacgagaacaaggtgctcgtgctcgataccgactacaagaagtaccttctcgtctgcatggaaaactccgctgagccagagcaatctcttgtttgccaatgccttgtgaggaccccagaggttgacgatgaagctcttgagaagttcgacaaggctctcaaggctttgcctatgcacatccgccttagcttcaacccaactcagcttgaggaacagtgccacatcaaggatgagctttaaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgactcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagt

SEQ ID NO: 780 - DNA, 3655 bp, synthetic construct
  misc_feature 1..3655: note = Construct AR15-37
  source 1..3655: mol_type = other DNA
aacacaagcttcaagttttaaaaggaaaaatgtcagccaaaaactttaaataaaatggtaacaaggaaattattcaaaaattacaaacctcgtcaaaataggaaagaaaaaaagtttagggatttagaaaaaacatcaatctagttccaccttattttatagagagaagaaactaatatataagaactaaaaaacagaagaatagaaaaaaaaagtattgacaggaaagaaaaagtagctgtatgcttataagtactttgaggatttgaattctctcttataaaacacaaacacaatttttagattttatttaaataatcatcaatccgattataattatttatatatttttctattttcaaagaagtaaatcatgagcttttccaactcaacatctattttttttctctcaacctttttcacatcttaagtagtctcaccctttatatatataacttatttcttaccttttacattatgtaacttttatcaccaaaaccaacaactttaaaattttattaaatagactccacaagtaacttgacactcttacattcatcgacattaacttttatctgttttataaatattattgtgatataatttaatcaaaataaccacaaactttcataaaaggttcttattaagcatggcatttaataagcaaaaacaactcaatcactttcatataggaggtagcctaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttcacaactcaaacattctctccattggtccttaaacactcatcagtcatcaccgaattctctaaaagagatctttttctgctctttgaagaaagaagggtctttgcttgattttggagatggccaagctagttttttccctttgttttctgcttttcagtggctgctgcttcgctcttattgtgacccaaaccatgaagggcctcgacattcaaaaggttgccggaacctggtactcccttgctatggctgcttccgatatctccttgctcgatgctcaatccgctccacttagggtgtacgtggaagagttgaagccaactccagagggcgatcttgagatcttgcttcaaaagtgggagaacgatgagtgcgcccagaagaagattatcgccgaaaagaccaagattcccgccgtgttcaagatcgatgctctcaacgagaacaaggtgctcgtgctcgataccgactacaagaagtaccttctcgtctgcatggaaaactccgctgagccagagcaatctcttgtttgccaatgccttgtgaggaccccagaggttgacgatgaagctcttgagaagttcgacaaggctctcaaggctttgcctatgcacatccgccttagcttcaacccaactcagcttgaggaacagtgccacatcaaggatgagctttaaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgactcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagt

SEQ ID NO: 781 - DNA, 3824 bp, synthetic construct
  misc_feature 1..3824: note = Construct AR15-39
  source 1..3824: mol_type = other DNA
aacacaagcttcaagttttaaaaggaaaaatgtcagccaaaaactttaaataaaatggtaacaaggaaattattcaaaaattacaaacctcgtcaaaataggaaagaaaaaaagtttagggatttagaaaaaacatcaatctagttccaccttattttatagagagaagaaactaatatataagaactaaaaaacagaagaatagaaaaaaaaagtattgacaggaaagaaaaagtagctgtatgcttataagtactttgaggatttgaattctctcttataaaacacaaacacaatttttagattttatttaaataatcatcaatccgattataattatttatatatttttctattttcaaagaagtaaatcatgagcttttccaactcaacatctattttttttctctcaacctttttcacatcttaagtagtctcaccctttatatatataacttatttcttaccttttacattatgtaacttttatcaccaaaaccaacaactttaaaattttattaaatagactccacaagtaacttgacactcttacattcatcgacattaacttttatctgttttataaatattattgtgatataatttaatcaaaataaccacaaactttcataaaaggttcttattaagcatggcatttaataagcaaaaacaactcaatcactttcatataggaggtagcctaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttcacaactcaaacattctctccattggtccttaaacactcatcagtcatcaccatgcagattttcgtgaagaccttaacggggaagacgatcaccctagaggttgagtcttccgacaccatcgacaatgtcaaagccaagatccaggacaaggaagggatacccccagaccagcagcgtttgattttcgccggaaagcagcttgaggatggtcgtactcttgccgactacaacatccagaaggagtcaactctccatctcgtgctccgtctccgtggtggtggttccatggccaagctagttttttccctttgttttctgcttttcagtggctgctgcttcgctcttattgtgacccaaaccatgaagggcctcgacattcaaaaggttgccggaacctggtactcccttgctatggctgcttccgatatctccttgctcgatgctcaatccgctccacttagggtgtacgtggaagagttgaagccaactccagagggcgatcttgagatcttgcttcaaaagtgggagaacgatgagtgcgcccagaagaagattatcgccgaaaagaccaagattcccgccgtgttcaagatcgatgctctcaacgagaacaaggtgctcgtgctcgataccgactacaagaagtaccttctcgtctgcatggaaaactccgctgagccagagcaatctcttgtttgccaatgccttgtgaggaccccagaggttgacgatgaagctcttgagaagttcgacaaggctctcaaggctttgcctatgcacatccgccttagcttcaacccaactcagcttgaggaacagtgccacatcaaggatgagctttaaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgactcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagt

SEQ ID NO: 782 - DNA, 10063 bp, synthetic construct
  misc_feature 1..10063: note = Construct AR15-00
  source 1..10063: mol_type = other DNA
gcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgcccgatcggtttaaaccacacaggaaacagctatgacatgattacgaattcggccaaagcacatagttatcgatttaaatttcatcgaagagattaatatcgaataatcatatacatactttaaatacataacaaattttaaatacatatatctggtatataattaattttttaaagtcatgaagtatgtatcaaatacaaatatggaaaaaattaactattcataatttaaaaaatagaaaagatacatctagtgaaattaggtgcatgtatcaaatacattaggaaaagggcatatatcttgatctagataattaacgattttgatttatgtataatttccaaatgaaggtttatatctacttcagaaataacaatatacttttatcagaacattcaacaaagcaacaaccaactagagtgaaaaatacacattgttctctagacatacaaaattgagaaaagaatctcaaaatttagagaaacaaatctgaatttctagaagaaaaaaataattatgcactttgctattgctcgaaaaataaatgaaagaaattagacttttttaaaagatgttagactagatatactcaaaagctattaaaggagtaatattcttcttacattaagtattttagttacagtcctgtaattaaagacacattttagattgtatctaaacttaaatgtatctagaatacatatatttgattgcatcatatccatgtatccgacacaccaattctcataaaaaacgtaatatcctaaactaatttatccttcaagtcaacctaagcccaatatacattttcatctctaaaggcccaagtggcacaaaatgtcaggcccaattacgaagaaaagggcttgtaaaaccctaataaagtggcactggcagagcttacactctcattccatcaacaaagaaaccctaaaagccgcagcgccactgatttctctcctccaggcgaagtgatcaatggcggcggcaacaacaacaacaacaacatcttcttcgatctccttctccaccaaaccatctccttcctcctccaaatcaccattaccaatctccagattctccctcccattctccctaaaccccaacaaatcatcctcctcctcccgccgccgcggtatcaaatccagctctccctcctccatctccgccgtgctcaacacaaccaccaatgtcacaaccactccctctccaaccaaacctaccaaacccgaaacattcatctcccgattcgctccagatcaaccccgcaaaggcgctgatatcctcgtcgaagctttagaacgtcaaggcgtagaaaccgtattcgcttaccctggaggtgcatcaatggagattcaccaagccttaacccgctcttcctcaatccgtaacgtccttcctcgtcacgaacaaggaggtgtattcgcagcagaaggatacgctcgatcctcaggtaaaccaggtatctgtatagccacttcaggtcccggagctacaaatctcgttagcggattagccgatgcgttgttagatagtgttcctcttgtagcaatcacaggacaagtccctcgtcgtatgattggtacagatgcgtttcaagagactccgattgttgaggtaacgcgttcgattacgaagcataactatcttgtgatggatgttgaagatatccctaggattattgaggaagctttctttttagctacttctggtagacctggacctgttttggttgatgttcctaaagatattcaacaacagcttgcgattcctaattgggaacaggctatgagattacctggttatatgtctaggatgcctaaacctccggaagattctcatttggagcagattgttaggttgatttctgagtctaagaagcctgtgttgtatgttggtggtggttgtttgaattctagcgatgaattgggtaggtttgttgagcttacggggatccctgttgcgagtacgttgatggggctgggatcttatccttgtgatgatgagttgtcgttacatatgcttggaatgcatgggactgtgtatgcaaattacgctgtggagcatagtgatttgttgttggcgtttggggtaaggtttgatgatcgtgtcacgggtaagcttgaggcttttgctagtagggctaagattgttcatattgatattgactcggctgagattgggaagaataagactcctcatgtgtctgtgtgtggtgatgttaagctggctttgcaagggatgaataaggttcttgagaaccgagcggaggagcttaagcttgattttggagtttggaggaatgagttgaacgtacagaaacagaagtttccgttgagctttaagacgtttggggaagctattcctccacagtatgcgattaaggtccttgatgagttgactgatggaaaagccataataagtactggtgtcgggcaacatcaaatgtgggcggcgcagttctacaattacaagaaaccaaggcagtggctatcatcaggaggccttggagctatgggatttggacttcctgctgcgattggagcgtctgttgctaaccctgatgcgatagttgtggatattgacggagatggaagctttataatgaatgtgcaagagctagccactattcgtgtagagaatcttccagtgaaggtacttttattaaacaaccagcatcttggcatggttatgcaatgggaagatcggttctacaaagctaaccgagctcacacatttctcggggatccggctcaggaggacgagatattcccgaacatgttgctgtttgcagcagcttgcgggattccagcggcgagggtgacaaagaaagcagatctccgagaagctattcagacaatgctggatacaccaggaccttacctgttggatgtgatttgtccgcaccaagaacatgtgttgccgatgatcccgaatggtggcactttcaacgatgtcataacggaaggagatggccggattaaatactgatcactgattttaatgtttagcaaatgtcttatcagttttctctttttgtcgaacggtaatttagagttttttttgctatatggattttcgtttttgatgtatgtgacaaccctcgggattgttgatttatttcaaaactaagagtttttgtcttattgttctcgtctattttggaatatcaatcttagttttatatcttttctagttctctacgtgttaaatgttcaacacactagcaatttggcctgccagcgtatggattatggaactatcaagtgtgtgggatcgataaatatgcttctcaggaatttgagattttacagtctttatgctcattgggttgagtataatatagtaaaaaaatagtaaatttaagcaataatgttaggtgctatgtgtctgtcgagactattggccggtacccggggatcctctagagtcgacctgcaggcatgcaagctagggataacagggtaatagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccggga

SEQ ID NO: 783 - DNA, 3358 bp, synthetic construct
  misc_feature 1..3358: note = Selection marker in AR15-00
  source 1..3358: mol_type = other DNA
ggccaaagcacatagttatcgatttaaatttcatcgaagagattaatatcgaataatcatatacatactttaaatacataacaaattttaaatacatatatctggtatataattaattttttaaagtcatgaagtatgtatcaaatacaaatatggaaaaaattaactattcataatttaaaaaatagaaaagatacatctagtgaaattaggtgcatgtatcaaatacattaggaaaagggcatatatcttgatctagataattaacgattttgatttatgtataatttccaaatgaaggtttatatctacttcagaaataacaatatacttttatcagaacattcaacaaagcaacaaccaactagagtgaaaaatacacattgttctctagacatacaaaattgagaaaagaatctcaaaatttagagaaacaaatctgaatttctagaagaaaaaaataattatgcactttgctattgctcgaaaaataaatgaaagaaattagacttttttaaaagatgttagactagatatactcaaaagctattaaaggagtaatattcttcttacattaagtattttagttacagtcctgtaattaaagacacattttagattgtatctaaacttaaatgtatctagaatacatatatttgattgcatcatatccatgtatccgacacaccaattctcataaaaaacgtaatatcctaaactaatttatccttcaagtcaacctaagcccaatatacattttcatctctaaaggcccaagtggcacaaaatgtcaggcccaattacgaagaaaagggcttgtaaaaccctaataaagtggcactggcagagcttacactctcattccatcaacaaagaaaccctaaaagccgcagcgccactgatttctctcctccaggcgaagtgatcaatggcggcggcaacaacaacaacaacaacatcttcttcgatctccttctccaccaaaccatctccttcctcctccaaatcaccattaccaatctccagattctccctcccattctccctaaaccccaacaaatcatcctcctcctcccgccgccgcggtatcaaatccagctctccctcctccatctccgccgtgctcaacacaaccaccaatgtcacaaccactccctctccaaccaaacctaccaaacccgaaacattcatctcccgattcgctccagatcaaccccgcaaaggcgctgatatcctcgtcgaagctttagaacgtcaaggcgtagaaaccgtattcgcttaccctggaggtgcatcaatggagattcaccaagccttaacccgctcttcctcaatccgtaacgtccttcctcgtcacgaacaaggaggtgtattcgcagcagaaggatacgctcgatcctcaggtaaaccaggtatctgtatagccacttcaggtcccggagctacaaatctcgttagcggattagccgatgcgttgttagatagtgttcctcttgtagcaatcacaggacaagtccctcgtcgtatgattggtacagatgcgtttcaagagactccgattgttgaggtaacgcgttcgattacgaagcataactatcttgtgatggatgttgaagatatccctaggattattgaggaagctttctttttagctacttctggtagacctggacctgttttggttgatgttcctaaagatattcaacaacagcttgcgattcctaattgggaacaggctatgagattacctggttatatgtctaggatgcctaaacctccggaagattctcatttggagcagattgttaggttgatttctgagtctaagaagcctgtgttgtatgttggtggtggttgtttgaattctagcgatgaattgggtaggtttgttgagcttacggggatccctgttgcgagtacgttgatggggctgggatcttatccttgtgatgatgagttgtcgttacatatgcttggaatgcatgggactgtgtatgcaaattacgctgtggagcatagtgatttgttgttggcgtttggggtaaggtttgatgatcgtgtcacgggtaagcttgaggcttttgctagtagggctaagattgttcatattgatattgactcggctgagattgggaagaataagactcctcatgtgtctgtgtgtggtgatgttaagctggctttgcaagggatgaataaggttcttgagaaccgagcggaggagcttaagcttgattttggagtttggaggaatgagttgaacgtacagaaacagaagtttccgttgagctttaagacgtttggggaagctattcctccacagtatgcgattaaggtccttgatgagttgactgatggaaaagccataataagtactggtgtcgggcaacatcaaatgtgggcggcgcagttctacaattacaagaaaccaaggcagtggctatcatcaggaggccttggagctatgggatttggacttcctgctgcgattggagcgtctgttgctaaccctgatgcgatagttgtggatattgacggagatggaagctttataatgaatgtgcaagagctagccactattcgtgtagagaatcttccagtgaaggtacttttattaaacaaccagcatcttggcatggttatgcaatgggaagatcggttctacaaagctaaccgagctcacacatttctcggggatccggctcaggaggacgagatattcccgaacatgttgctgtttgcagcagcttgcgggattccagcggcgagggtgacaaagaaagcagatctccgagaagctattcagacaatgctggatacaccaggaccttacctgttggatgtgatttgtccgcaccaagaacatgtgttgccgatgatcccgaatggtggcactttcaacgatgtcataacggaaggagatggccggattaaatactgatcactgattttaatgtttagcaaatgtcttatcagttttctctttttgtcgaacggtaatttagagttttttttgctatatggattttcgtttttgatgtatgtgacaaccctcgggattgttgatttatttcaaaactaagagtttttgtcttattgttctcgtctattttggaatatcaatcttagttttatatcttttctagttctctacgtgttaaatgttcaacacactagcaatttggcctgccagcgtatggattatggaactatcaagtgtgtgggatcgataaatatgcttctcaggaatttgagattttacagtctttatgctcattgggttgagtataatatagtaaaaaaatagtaaatttaagcaataatgttaggtgctatgtgtctgtcgagactattggcc

SEQ ID NO: 784 - DNA, 10676 bp, synthetic construct
  misc_feature 1..10676: note = Construct AR07-00
  source 1..10676: mol_type = other DNA
aattctaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaaatagagcttgtcgatcgttaattaattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgcatgcagtggccacggaagcagaaattctgaatgaacagaacttttgcatgaagattgaatctttcttaagcaccaacatagaattagcagagttcttcaacttcttggaaccgaagacaagaaagctagagcttttaggaacctgtggcttatggaaattagagttgggattgagagtctggatgccttgtgccatattgttaatctgggccatggtagactcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcggtacccggggatcctctagagtcgacctgcaggcatgcaagctagggataacagggtaatgggcccaatataacaacgacgtcgtaacagataaagcgaagcttgaaggtgcatgtgactccgtcaagattacgaaaccgccaactaccacgcaaattgcaattctcaatttcctagaaggactctccgaaaatgcatccaataccaaatattacccgtgtcataggcaccaagtgacaccatacatgaacacgcgtcacaatatgactggagaagggttccacaccttatgctataaaacgccccacacccctcctccttccttcgcagttcaattccaatatattccattctctctgtgtatttccctacctctcccttcaaggttagtcgatttcttctgtttttcttcttcgttctttccatgaattgtgtatgttctttgatcaatacgatgttgatttgattgtgttttgtttggtttcatcgatcttcaattttcataatcagattcagcttttattatctttacaacaacgtccttaatttgatgattctttaatcgtagatttgctctaattagagctttttcatgtcagatccctttacaacaagccttaattgttgattcattaatcgtagattagggcttttttcattgattacttcagatccgttaaacgtaaccatagatcagggctttttcatgaattacttcagatccgttaaacaacagccttattttttatacttctgtggtttttcaagaaattgttcagatccgttgacaaaaagccttattcgttgattctatatcgtttttcgagagatattgctcagatctgttagcaactgccttgtttgttgattctattgccgtggattagggttttttttcacgagattgcttcagatccgtacttaagattacgtaatggattttgattctgatttatctgtgattgttgactcgacaggtcgacatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaacccggggttcgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacactagggataacagggtaatagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgcccgatcggtttaaaccacacaggaaacagctatgaccatgattacg

SEQ ID NO: 785 - DNA, 678 bp, synthetic construct
  misc_feature 1..678: note = CaMV35S promoter
  source 1..678: mol_type = other DNA
tcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctca

SEQ ID NO: 786 - DNA, 917 bp, synthetic construct
  misc_feature 1..917: note = GmU3 promoter
  source 1..917: mol_type = other DNA
gggcccaatataacaacgacgtcgtaacagataaagcgaagcttgaaggtgcatgtgactccgtcaagattacgaaaccgccaactaccacgcaaattgcaattctcaatttcctagaaggactctccgaaaatgcatccaataccaaatattacccgtgtcataggcaccaagtgacaccatacatgaacacgcgtcacaatatgactggagaagggttccacaccttatgctataaaacgccccacacccctcctccttccttcgcagttcaattccaatatattccattctctctgtgtatttccctacctctcccttcaaggttagtcgatttcttctgtttttcttcttcgttctttccatgaattgtgtatgttctttgatcaatacgatgttgatttgattgtgttttgtttggtttcatcgatcttcaattttcataatcagattcagcttttattatctttacaacaacgtccttaatttgatgattctttaatcgtagatttgctctaattagagctttttcatgtcagatccctttacaacaagccttaattgttgattcattaatcgtagattagggcttttttcattgattacttcagatccgttaaacgtaaccatagatcagggctttttcatgaattacttcagatccgttaaacaacagccttattttttatacttctgtggtttttcaagaaattgttcagatccgttgacaaaaagccttattcgttgattctatatcgtttttcgagagatattgctcagatctgttagcaactgccttgtttgttgattctattgccgtggattagggttttttttcacgagattgcttcagatccgtacttaagattacgtaatggattttgattctgatttatctgtgattgttgactcgacag

SEQ ID NO: 787 - DNA, 922 bp, synthetic construct
  misc_feature 1..922: note = StUbi3 promoter
  source 1..922: mol_type = other DNA
ggccaaagcacatagttatcgatttaaatttcatcgaagagattaatatcgaataatcatatacatactttaaatacataacaaattttaaatacatatatctggtatataattaattttttaaagtcatgaagtatgtatcaaatacaaatatggaaaaaattaactattcataatttaaaaaatagaaaagatacatctagtgaaattaggtgcatgtatcaaatacattaggaaaagggcatatatcttgatctagataattaacgattttgatttatgtataatttccaaatgaaggtttatatctacttcagaaataacaatatacttttatcagaacattcaacaaagcaacaaccaactagagtgaaaaatacacattgttctctagacatacaaaattgagaaaagaatctcaaaatttagagaaacaaatctgaatttctagaagaaaaaaataattatgcactttgctattgctcgaaaaataaatgaaagaaattagacttttttaaaagatgttagactagatatactcaaaagctattaaaggagtaatattcttcttacattaagtattttagttacagtcctgtaattaaagacacattttagattgtatctaaacttaaatgtatctagaatacatatatttgattgcatcatatccatgtatccgacacaccaattctcataaaaaacgtaatatcctaaactaatttatccttcaagtcaacctaagcccaatatacattttcatctctaaaggcccaagtggcacaaaatgtcaggcccaattacgaagaaaagggcttgtaaaaccctaataaagtggcactggcagagcttacactctcattccatcaacaaagaaaccctaaaagccgcagcgccactgatttctctcctccaggcgaag

SEQ ID NO: 788 - DNA, 1940 bp, synthetic construct
  misc_feature 1..1940: note = Selection marker in AR07-00
  source 1..1940: mol_type = other DNA
taattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaaatagagcttgtcgatcgttaattaattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgcatgcagtggccacggaagcagaaattctgaatgaacagaacttttgcatgaagattgaatctttcttaagcaccaacatagaattagcagagttcttcaacttcttggaaccgaagacaagaaagctagagcttttaggaacctgtggcttatggaaattagagttgggattgagagtctggatgccttgtgccatattgttaatctgggccatggtagactcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctca

SEQ ID NO: 789 - DNA, 1937 bp, synthetic construct
  misc_feature 1..1937: note = Reporter gene in AR07-00
  source 1..1937: mol_type = other DNA
gggcccaatataacaacgacgtcgtaacagataaagcgaagcttgaaggtgcatgtgactccgtcaagattacgaaaccgccaactaccacgcaaattgcaattctcaatttcctagaaggactctccgaaaatgcatccaataccaaatattacccgtgtcataggcaccaagtgacaccatacatgaacacgcgtcacaatatgactggagaagggttccacaccttatgctataaaacgccccacacccctcctccttccttcgcagttcaattccaatatattccattctctctgtgtatttccctacctctcccttcaaggttagtcgatttcttctgtttttcttcttcgttctttccatgaattgtgtatgttctttgatcaatacgatgttgatttgattgtgttttgtttggtttcatcgatcttcaattttcataatcagattcagcttttattatctttacaacaacgtccttaatttgatgattctttaatcgtagatttgctctaattagagctttttcatgtcagatccctttacaacaagccttaattgttgattcattaatcgtagattagggcttttttcattgattacttcagatccgttaaacgtaaccatagatcagggctttttcatgaattacttcagatccgttaaacaacagccttattttttatacttctgtggtttttcaagaaattgttcagatccgttgacaaaaagccttattcgttgattctatatcgtttttcgagagatattgctcagatctgttagcaactgccttgtttgttgattctattgccgtggattagggttttttttcacgagattgcttcagatccgtacttaagattacgtaatggattttgattctgatttatctgtgattgttgactcgacaggtcgacatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaacccggggttcgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacact

SEQ ID NO: 790-809 - skipped (20 entries)

SEQ ID NO: 810 - protein, 386 aa, Meleagris gallopavo
MGSIGAVSMEFCFDVFKELKVHHANENIFYSPFTIISALAMVYLGAKDSTRTQINKVVRFDKLPGFGDSVEAQCGTSVNVHSSLRDILNQITKPNDVYSFSLASRLYAEETYPILPEYLQCVKELYRGGLESINFQTAADQARGLINSWVESQTNGMIKNVLQPSSVDSQTAMVLVNAIVFKGLWEKAFKDEDTQAIPFRVTEQESKPVQMMYQIGLFKVASMASEKMKILELPFASGTMSMWVLLPDEVSGLEQLETTISFEKMTEWISSNIMEERRIKVYLPRMKMEEKYNLTSVLMAMGITDLFSSSANLSGISSAGSLKISQAVHAAYAEIYEAGREVIGSAEAGADATSVSEEFRVDHPFLYCIKHNLTNSILFFGRCISP

SEQ ID NO: 811 - protein, 386 aa, Meleagris gallopavo
MGSIGAVSMEFCFDVFKELKVHHANENIFYSPFTIISALAMVYLGAKDSTRTQINKVVRFDKLPGFGDSVEAQCGTSVNVHSSLRDILNQITKPNDVYSFSLASRLYAEETYPILPEYLQCVKELYRGGLESINFQTAADQARGLINSWVESQTNGMIKNVLQPSSVDSQTAMVLVNAIVFKGLWEKAFKDEDTQAIPFRVTEQESKPVQMMYQIGLFKVASMASEKMKILELPFASGTMSMWVLLPDEVSGLEQLETTISFEKMTEWISSNIMEERRIKVYLPRMKMEEKYNLTSVLMAMGITDLFSSSANLSGISSAGSLKISQAAHAAYAEIYEAGREVIGSAEAGADATSVSEEFRVDHPFLYCIKHNLTNSILFFGRCISP

SEQ ID NO: 812 - protein, 383 aa, Coturnix japonica
MGSIGAASMEFCFDVFKELKVHHANDNMLYSPFAILSTLAMVFLGAKDSTRTQINKVVHFDKLPGFGDSIEAQCGTSVNVHSSLRDILNQITKQNDAYSFSLASRLYAQETYTVVPEYLQCVKELYRGGLESVNFQTAADQARGLINAWVESQTNGIIRNILQPSSVDSQTAMVLVNAIAFKGLWEKAFKAEDTQTIPFRVTEQESKPVQMMYQIGSFKVASMASEKMKILELPFASGTMSMLVLLPDDVSGLEQLESIISFEKLTEWTSSSIMEERKVKVYLPRMKMEEKYNLTSLLMAMGITDLFSSSANLSGISSVGSLKISQAVHAAHAEINEAGRDVVGSAEAGVDATEEFRADHPFLFCVKHIETNAILLFGRCVSP

SEQ ID NO: 813 - protein, 417 aa, Bambusicola thoracicus
YYRVPCMVLCTAFHPYIFIVLLFALDNSEFTMGSIGAVSMEFCFDVFKELRVHHPNENIFFCPFAIMSAMAMVYLGAKDSTRTQINKVIRFDKLPGFGDSTEAQCGKSANVHSSLKDILNQITKPNDVYSFSLASRLYADETYSIQSEYLQCVNELYRGGLESINFQTAADQARELINSWVESQTNGIIRNVLQPSSVDSQTAMVLVNAIVFRGLWEKAFKDEDTQTMPFRVTEQESKPVQMMYQIGSFKVASMASEKMKILELPLASGTMSMLVLLPDEVSGLEQLETTISFEKLTEWTSSNVMEERKIKVYLPRMKMEEKYNLTSVLMAMGITDLFRSSANLSGISLAGNLKISQAVHAAHAEINEAGRKAVSSAEAGVDATSVSEEFRADRPFLFCIKHIATKVVFFFGRYTSP

SEQ ID NO: 814 - protein, 386 aa, Numida Meleagris
MASIGAVSTEFCVDVYKELRVHHANENIFYSPFTIISTLAMVYLGAKDSTRTQINKVVRFDKLPGFGDSIEAQCGTSVNVHSSLRDILNQITKPNDVYSFSLASRLYAEETYPILPEYLQCVKELYRGGLESINFQTAADQARELINSWVESQTSGIIKNVLQPSSVNSQTAMVLVNAIYFKGLWERAFKDEDTQAIPFRVTEQESKPVQMMSQIGSFKVASVASEKVKILELPFVSGTMSMLVLLPDEVSGLEQLESTISTEKLTEWTSSSIMEERKIKVFLPRMRMEEKYNLTSVLMAMGMTDLFSSSANLSGISSAESLKISQAVHAAYAEIYEAGREVVSSAEAGVDATSVSEEFRVDHPFLLCIKHNPTNSILFFGRCISP

SEQ ID NO: 815 - protein, 411 aa, Numida Meleagris
MALCKAFHPYIFIVLLFDVDNSAFTMASIGAVSTEFCVDVYKELRVHHANENIFYSPFTIISTLAMVYLGAKDSTRTQINKVVRFDKLPGFGDSIEAQCGTSVNVHSSLRDILNQITKPNDVYSFSLASRLYAEETYPILPEYLQCVKELYRGGLESINFQTAADQARELINSWVESQTSGIIKNVLQPSSVNSQTAMVLVNAIYFKGLWERAFKDEDTQAIPFRVTEQESKPVQMMSQIGSFKVASVASEKVKILELPFVSGTMSMLVLLPDEVSGLEQLESTISTEKLTEWTSSSIMEERKIKVFLPRMRMEEKYNLTSVLMAMGMTDLFSSSANLSGISSAESLKISQAVHAAYAEIYEAGREVVSSAEAGVDATSVSEEFRVDHPFLLCIKHNPTNSILFFGRCISP

SEQ ID NO: 816 - protein, 411 aa, Odontophorus gujanensis
RILCMAFHPYIFIVLLFAPDNSEFTMGSIGAVSTEFCFDVFKELKVHHANENIFYSPFTIISALAMVYLGAKDSTRTQINKVVRFDKLPGFGDSIEAQCGTSVNVHSSLRDILNQITKPNDFYSFSLASRLYADEAYPILPEYLQCVKELYRGGLESINFQTAADQARELINSWVESQTSGIIRNVLQPSSVDSQTAIVLVNAIYFKALWKKGFKNEDTQAIPFRVTEQESKSVQMMQQIGTFKVASVASEKMKILELPFASGTMSMWVLLPDEVSDLEQLETTISFEKLTEWTSSNIMEERKIKVFLPRMKMEEKYNLTSVLMAMGMTDLFSSSANLSGISSAESLKISQAVHAAYAEIYEAGSEVVGSAEAGVDATSATEEFRVDRPFLFCIKHNPTNSILFFGRCISP

SEQ ID NO: 817 - protein, 383 aa, Coturnix japonica
MGSIGAASMEFCFDVFKELKVHHANDNMLYSPFAILSTLAMVFLGAKDSTRTQINKVVHFDKLPGFGDSIEAQCGTSANVHSSLRDILNQITKQNDAYSFSLASRLYAQETYTVVPEYLQCVKELYRGGLESVNFQTAADQARGLINAWVESQTNGIIRNILQPSSVDSQTAMVLVNAIAFKGLWEKAFKAEDTQTIPFRVTEQESKPVQMMHQIGSFKVASMASEKMKILELPFASGTMSMLVLLPDDVSGLEQLESTISFEKLTEWTSSSIMEERKVKVYLPRMKMEEKYNLTSLLMAMGITDLFSSSANLSGISSVGSLKISQAVHAAYAEINEAGRDVVGSAEAGVDATEEFRADHPFLFCVKHIETNAILLFGRCVSP

SEQ ID NO: 818 - protein, 408 aa, Coturnix japonica
MGLCTAFHPYIFIVLLFALDNSEFTMGSIGAASMEFCFDVFKELKVHHANDNMLYSPFAILSTLAMVFLGAKDSTRTQINKVVHFDKLPGFGDSIEAQCGTSANVHSSLRDILNQITKQNDAYSFSLASRLYAQETYTVVPEYLQCVKELYRGGLESVNFQTAADQARGLINAWVESQTNGIIRNILQPSSVDSQTAMVLVNAIAFKGLWEKAFKAEDTQTIPFRVTEQESKPVQMMHQIGSFKVASMASEKMKILELPFASGTMSMLVLLPDDVSGLEQLESTISFEKLTEWTSSSIMEERKVKVYLPRMKMEEKYNLTSLLMAMGITDLFSSSANLSGISSVGSLKISQAVHAAYAEINEAGRDVVGSAEAGVDATEEFRADHPFLFCVKHIETNAILLFGRCVSP

SEQ ID NO: 819 - protein, 383 aa, Coturnix coturnix
MGSIGAASMEFCFDVFKELKVHHANDNMLYSPFAILSTLAMVFLGAKDSTRTQINKVVHFDKLPGFGDSIEAQCGTSANVHSSLRDILNQITKQNDAYSFSLASRLYAQETYTVVPEYLQCVKELYRGGLESVNFQTAADQARGLINAWVESQTNGIIRNILQPSSVDSQTAMVLVNAIAFKGLWEKAFKAEDTQTIPFRVTEQESKPVQMMHQIGSFKVASMASEKMKILELPFASGTMSMLVLLPDDVSGLEQLESTISFEKLTEWTSSSIMEERKVKVYLPRMKMEEKYNLTSLLMAMGITDLFSSSANLSGISSVGSLKIPQAVHAAYAEINEAGRDVVGSAEAGVDATEEFRADHPFLFCVKHIETNAILLFGRCVSP

SEQ ID NO: 820 - protein, 388 aa, Phasianus colchicus
MGSIGAVSMEFCFDVLKELKVHHANENYFYAPFTMFSALAMIYLGAKDSTRAQINKVVRFDKLPGFGDSIEAQCGTSADPQVHSSLRDILNQITKPNDAYSFSLASRLYADEKYSIVPEYLKCVKELYRGDVESINFQTAADQARGLINSWVESQTNGMIKNVLQPSSVDSQTAMVLVNAVVFKGLWEKAFKEEDTQAIPFRVTEQESKPVQMMHQIGLFKVASVPSEKMKILELPFASGTMSMWVLLPDEVSGLEQLETTISFEKMTEWTSSNIMEERKIRVYLPRMKMEEKYNLTSILMAMGMTDLFSSSANLSGISSVGSLKISQAVHAAYAEIYEAGREVAGSAEAAMDATSVSEEFRVDHPFLYCIKHNPSNTLLFLGRCIFP

SEQ ID NO: 821 - protein, 413 aa, Phasianus colchicus
MALCTAFHPYVFIILLFALDNSEFTMGSIGAVSMEFCFDVLKELKVHHANENYFYAPFTMFSALAMIYLGAKDSTRAQINKVVRFDKLPGFGDSIEAQCGTSADPQVHSSLRDILNQITKPNDAYSFSLASRLYADEKYSIVPEYLKCVKELYRGDVESINFQTAADQARGLINSWVESQTNGMIKNVLQPSSVDSQTAMVLVNAVVFKGLWEKAFKEEDTQAIPFRVTEQESKPVQMMHQIGLFKVASVPSEKMKILELPFASGTMSMWVLLPDEVSGLEQLETTISFEKMTEWTSSNIMEERKIRVYLPRMKMEEKYNLTSILMAMGMTDLFSSSANLSGISSVGSLKISQAVHAAYAEIYEAGREVAGSAEAAMDATSVSEEFRVDHPFLYCIKHNPSNTLLFLGRCIFP

SEQ ID NO: 822 - protein, 411 aa, Penelope pileate
IALRTAYPPYIVIVLLFALDNSEFTMASIGAVSTEFCFNVFRELKVQHANENIFYCPFTIFSALAFAYLGAKENTRTQINKVAHFDKLPGFGDSIEAQCGTSANVHSSLRDILNQITKPSDNYSLSLASRLYVDERYPILPEYLQCVKELYRGGVEPITFQTAADQARELINSWVESQTNGMIKNILQPSSVDSQTAMVLVNAVYFKGMWQKAFKNEDTQEMPFRITENESKPVQMMHQIGSFKIATVASEKLKILELPYASGMMSMLVLLPDQASGLEQLENTISFEKLNEWTSSNMVEERRIKVYLPRMKMEEKYNLTAVLTALGITDLFSPSANLSGISSAASLKISQAVHAAYAEIYEAGRDVVGSAEAGVDATSVTDEFRVDHPFLFCMKHNPSNSIVFLGKCVSP

SEQ ID NO: 823 - protein, 409 aa, Anseranas semipalmata
CTAFHHYIVIVLLLFALDNSDFTMGSIGAASAEFCFDVFKELKVHHANENICYSPLSIISALAMVYLGARDNTRTQIDKVVHFDQIPGFGESIESQCGTSVSVHSSLTDILTQITKPSDNYSFSLASRLYAEETYPILPEYLQCVKELYKGGLESISFQTAADQARELINSWVESQTNGIIKNILQPSSVDSQTAMVLVNAIYFKGMWEKAFKDENTQEMPFRVTEQESKPVQMMFQFGSFKVATVASEKVKILELPYASGMISMCVLLPDEVSGLEQIENTISLEKLTEWTSSNMMEERRMKVYLPRMKLEEQYNLTSVLMALGMTDLFSPSANLSGISSAESLKISEAVHAAYVEIYEAGREVVGSAEAGMDVSSVSEEFRVDHPFLFLIKHNPSNSILFFGRLISP

SEQ ID NO: 824 - protein, 412 aa, Chauna torquata
HYVCTAFHHHTVIVLLLFALDNSDFTMGSIGAASTEFCFDVFKELKVQHVNGNIFYSPLSIISALAMVYLGARDNTRTQIDKVVHFDKIPGFGESIEAQCGTSESVHSSLKDILTQITKPSDNFSLSLASRLYAEETYPILPEYLQCVKELYKGGLESVSFQTAADQARELISSWVESQTNGIIKNILQPSSVDSQTEMVLVNAIYFKGMWEKAFKDEDTQTMPFRITEQESKPMQMMYQVGSFKVAVVASEKMKILELPYASGMMSMWVLLPDEVSGLEQLETTISFEKLTEWTSSNMMEERRMKVYLPRMKMEEKYNLTSVLIALGMTDLFSSSANLSGISSAESLKMSEAVHAAYVEIYEAGREVVGSAEAGMDVTSVSEEFKADRPFLFLIKHNPTNSILFFGRWISP

SEQ ID NO: 825 - protein, 386 aa, Anas platyrhynchos
MGSIGAASTEFCFDVFRELRVQHVNENIFYSPFSIISALAMVYLGARDNTRTQIDKVVHFDKLPGFGESMEAQCGTSVSVHSSLRDILTQITKPSDNFSLSFASRLYAEETYAILPEYLQCVKELYKGGLESISFQTAADQARELINSWVESQTNGIIKNILQPSSVDSQTTMVLVNAIYFKGMWEKAFKDEDTQAMPFRMTEQESKPVQMMYQVGSFKVAMVTSEKMKILELPFASGMMSMFVLLPDEVSGLEQLESTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPTNSILFFGRWMSP

SEQ ID NO: 826 - protein, 386 aa, Anas platyrhynchos
  VARIANT 45: note = X is any amino acid
MGSIGAASTEFCFDVFRELRVQHVNENIFYSPFSIISALAMVYLXARDNTRTQIDKVVHFDKLPGFGESMEAQCGTSVSVHSSLRDILTQITKPSDNFSLSFASRLYAEETYAILPEYLQCVKELYKGGLESISFQTAADQARELINSWVESQTNGIIKNILQPSSVDSQTTMVLVNAIYFKGMWEKAFKDEDTQAMPFRMTEQESKPVQMMYQVGSFKVAMVTSEKMKILELPFASGMMSMFVLLPDEVSGLEQLESTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPTNSILFFGRWMSP

SEQ ID NO: 827 - protein, 386 aa, Cygnus atratus
MGSIGAASTEFCFDVFRELKVQHVNENIFYSPLSIISALAMVYLGARDNTRAQIDKVVHFDKIPGFGESMESQCGTSVSVHSSLRDILTEITKPSDNFSLSFASRLYAEETYTILPEYLQCVKELYKGGLESISFQTAADQARELINSWVESQTNGIIKNILQPSSVDSQTTMVLVNAIYFKGMWEKAFKDEDTQTMPFRMTEQESKPVQMMYQVGSFKVATVTSEKVKILELPFASGMMSMCVLLPDEVSGLEQLETTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPTNSILFFGRWISP

SEQ ID NO: 828 - protein, 386 aa, Anser cygnoides
MGSIGAASTEFCFDVFRELKVQHVNENIFYSPLSIISALAMVYLGARDNTRTQIDQVVHFDKIPGFGESMEAQCGTSVSVHSSLRDILTEITKPSDNFSLSFASRLYAEETYTILPEYLQCVKELYKGGLESISFQTAADQARELINSWVESQTNGIIKNILQPSSVDSQTTMVLVNAIYFKGMWEKAFKDEDTQTMPFRMTEQESKPVQMMYQVGSFKLATVTSEKVKILELPFASGMMSMCVLLPDEVSGLEQLETTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPSNSILFFGRWISP

SEQ ID NO: 829 - DNA, 714 bp, synthetic construct
  misc_feature 1..714: note = eGFP
  source 1..714: mol_type = other DNA
gtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaag

SEQ ID NO: 830 - DNA, 714 bp, synthetic construct
  misc_feature 1..714: note = eGFP1
  source 1..714: mol_type = other DNA
gtgagtaagggtgaagagctgttcacaggcgtcgttcccatccttgttgagttggatggagacgtgaacggccataagttctctgtgagtggtgagggagagggtgatgcaacttatggcaaattgacattgaagttcatatgtactactggcaaattgcccgtgccttggcccaccttggtaaccacattcacttatggggttcaatgtttctccaggtatccagatcacatgaagcagcatgatttctttaagtccgctatgcctgaaggttacgtccaggagcgcactatattttttaaagacgatggaaactataagaccagggctgaggtgaagtttgaaggtgacactctcgtcaaccgtatagaactgaaagggatagacttcaaggaggatggaaacattttggggcataagttggaatacaattataactcccataatgtctatataatggcagataagcagaagaatggcattaaagtaaacttcaaaataagacataacattgaagacggctcagtccagctcgccgaccattaccaacaaaacacacccataggagacggtcctgtcctgcttcccgacaatcactacctgtcaactcagtccgccctgtccaaggacccaaatgaaaagagagaccatatggtgcttctggaatttgtcacagccgcaggaatcacacatggcatggatgaactttataag

SEQ ID NO: 831 - DNA, 714 bp, synthetic construct
  misc_feature 1..714: note = eGFP2
  source 1..714: mol_type = other DNA
gtgtccaaaggagaagaactgttcactggtgtagtccctattctcgttgaactggacggcgatgtgaatggacataaattttccgtttccggggagggtgaaggagatgcaacctacggtaaactgactcttaaatttatatgtactacagggaagttgccagtaccctggccaacccttgttacaaccttcacatatggtgtacaatgtttctcccgctatcccgatcatatgaagcagcatgattttttcaagagtgctatgccagaaggttacgttcaggaaagaaccatcttcttcaaagacgacgggaactacaagaccagggcagaagtcaaattcgaaggggatactttggtaaatcgtattgagctgaaaggtattgatttcaaggaggacggtaacattcttggtcacaaattggaatataactataactcacataacgtgtatataatggccgataagcagaagaacggaattaaggtaaactttaagattagacacaacatagaggatggttcagtccaattggcagatcattatcaacagaacacacccataggagacggtcccgtcctgctgcctgataaccactacctctcaacacagtccgcactcagtaaagacccaaatgagaaacgtgaccacatggttcttctcgaatttgtgacagctgctggtatcactcatggtatggatgagctttacaag